(19) United States
(12) Patent Application Publication
(10) Pub. No.: US 2026/0062687 A1
(43) Pub. Date: Mar. 5, 2026

(54) BIOCATALYTIC METHOD FOR THE CONTROLLED DEGRADATION OF TERPENE COMPOUNDS

(71) Applicant: Firmenich SA, Satigny (CH)

(72) Inventors: Michel Schalk, Satigny (CH); Fabienne Deguerry, Satigny (CH); Daniel Solis Escalante, Satigny (CH); Pauline Anziani, Satigny (CH); Sabine Beccucci, Satigny (CH)

(21) Appl. No.: 19/292,515

(22) Filed: Aug. 6, 2025

Related U.S. Application Data
(63) Continuation of application No. 17/596,878, now Pat. No. 12,486,499.

(30) Foreign Application Priority Data
Jul. 10, 2019 (EP) 19000332.7
Nov. 13, 2019 (EP) 19208951.4

(51) Int. Cl. C12N 9/18; C12N 9/02; C12P 7/62

(52) U.S. Cl. CPC C12N 9/18; C12N 9/0069; C12P 7/62; C12Y 113/00

(57) ABSTRACT

Described herein are biocatalytic methods of producing terpene degradation products useful as starting material for the production of perfumery ingredients, such as, for example, ambrox. In particular, terpene degrading polypeptides (enal-cleaving polypeptides) and peptides converting terpenes compounds to oxygenated derivatives (oxygenases) and mutants and variants derived therefrom are described which may be applied in fully enzymatic multistep degradation pathways allowing the controlled, stepwise conversion and degradation of linear or cyclic terpene substrates. The biosynthetic strategies allow the fully biochemical synthesis of valuable terpene-derived compounds, like for example manooloxy or gamma ambrol. Also described herein are recombinant host organisms carrying the required set of genetic information for the functional expression of the set of enzymes necessary for catalyzing the combination of enzymatic conversion and degradation steps.

### CROSS-REFERENCE TO RELATED APPLICATIONS

This application is a continuation application of U.S. patent application Ser. No. 17/596,878, filed Dec. 20, 2021, which is a U.S. National Phase Application of International Patent Application No. PCT/EP2020/069217, filed Jul. 8, 2020, which claims priority to European Patent Application No. 19000332.7, filed Jul. 10, 2019, and which claims priority to European Patent Application No. 19208951.4, filed Nov. 13, 2019, the entire contents of which are hereby incorporated by reference herein.

### REFERENCE TO AN ELECTRONIC SEQUENCE LISTING

This application contains an electronic sequence listing. The content of the electronic sequence listing (revised_sequence_listing_36803-482.xml: Size: 366,489.6 bytes; and Date of Creation: Aug. 28, 2025) is herein incorporated by reference in its entirety.

### TECHNICAL FIELD

Provided herein are biocatalytic methods of producing terpene degradation products useful as starting material for the production of perfumery ingredients, such as, for example, ambrox. In particular novel terpene degrading polypeptides (enal-cleaving polypeptides) and novel peptides converting terpenes compounds to oxygenated derivatives (oxygenases) and mutants and variants derived therefrom are provided which may be applied in novel types of fully enzymatic multistep degradation pathways allowing the controlled, stepwise conversion and degradation of linear or cyclic terpene substrates. Said novel biosynthetic strategies allow the fully biochemical synthesis of valuable terpene-derived compounds, like for example manooloxy or gamma ambrol. The invention also provides recombinant host organisms carrying the required set of genetic information for the functional expression of the set of enzymes necessary for catalyzing the combination of enzymatic conversion and degradation steps.

### BACKGROUND

Terpenes are found in most organisms (microorganisms, animals and plants). These compounds are made up of five-carbon units, so-called isoprene units, and are classified by the number of these units present in their structure. Thus hemiterpenes, monoterpenes, sesquiterpenes and diterpenes are terpenes containing 5, 10, 15 and 20 carbon atoms (i.e. 1, 2, 3 and 4 isoprene units) respectively. Sesquiterpenes, for example, are widely found in the plant kingdom. Many sesquiterpene molecules are known for their flavor and fragrance properties and their cosmetic, medicinal and antimicrobial effects. Numerous sesquiterpene hydrocarbons and sesquiterpenoids have been identified.

Biosynthetic production of terpenes involves enzymes called terpene synthases. These enzymes convert an acyclic terpene precursor in one or more terpene products. In particular, diterpene synthases produce diterpenes by cyclization of the precursor geranylgeranyl diphosphate (GGPP). The cyclization of GGPP often requires two enzyme polypeptides, a type I and a type II diterpene synthase working in combination in two successive enzymatic reactions. The type II diterpene synthases catalyze a cyclization/rearrangement of GGPP initiated by the protonation of the terminal double bond of GGPP leading to a cyclic diterpene diphosphate intermediate. This intermediate is then further converted by a type I diterpene synthase catalyzing an ionization initiated cyclization.

Diterpene synthases are present in plants and other organisms and use substrates such as GGPP but they have different product profiles. Genes and cDNAs encoding diterpene synthases have been cloned and the corresponding recombinant enzymes characterized.

Enzymes that catalyze a specific or preferential cleavage or removal of diphosphate groups from terpene diphosphate intermediates, in particular from cyclic terpene diphosphate intermediates, like the diterpenes copalyl diphosphate (CPP) or labdendiol diphosphate (LPP) have only recently be described in an earlier European patent application. (EP Application Serial No. 18182783.3). By said enzymes the number or carbon atoms of the terpene diphosphate remains unchanged.

There is, however, the need terpene-derived compounds which may be considered as degradation products of terpene precursors, such as non-cyclic or cyclic sesquiterpenes or diterpenes, which in turn may the be further converted chemically and/or enzymatically into end product, to be applied for example as perfumery ingredients.

The problem to be solved by the present invention is to provide polypeptides which show the enzymatic terpene degrading activity or polypeptides which convert such terpenes into degradable derivatives.

Another problem to be solved by the present invention is the establishing of novel fully biocatalytic degradation pathway for generating defined terpene degradation products.

### SUMMARY

The above-mentioned problem could surprisingly be solved by providing a new class of polypeptides having enal-cleaving activity which allow for the first time the specific shortening of carbonyl-functionalized terpene compounds by 2 carbon atoms and respective bio catalytic processes. For example, the novel class of enzymes allows the conversion of the labdane-type compound copalal, which comprises a diterpene carbon skeleton and carries a terminal aldehyde group to the respective dinor-labdane compound manooloxy shortened by 2 carbon atoms, i.e. retaining a carbon skeleton composed of 18 carbon atoms.

The above-mentioned problem in an alternative approach could also surprisingly be solved by providing a new class of polypeptides having Baeyer-Villiger Monooxygenase (BVMO) activity which allow the specific oxidiation of terpene compounds to esters (Baeyer-Villiger oxygenation) and respective biocatalytic processes. For example, the novel class of BVMOs allows the conversion of the labdane-type compound copalal, which comprises a diterpene carbon skeleton and carries a terminal aldehyde group to the respective norlabdane formate ester. By said Baeyer-Villiger oxygenation the labdane compound may be easily converted to the respective norlabdane through the action of a polypeptide having esterase activity. This step results consequently in a shortening by one carbon atom. In case the terminal aldehyde group is replaced by a terminal keto group a shortening in the same manner but now by more than one carbonate is possible. Repetition of the combination of BVMO-catalysed oxygenation step and esterase-catalyzed cleavage step, allows the stepwise shortening of the hydrocarbon chain of the terpene molecule.

Combinations of degradation steps catalyzed by the above enal-cleaving enzymes and BVMO enzymes allow the construction of completely new biochemical degradation pathways applicable a greater variety of carbonyl functionalized chemical compounds, in particular cyclic or non-cyclic terpenes or terpenoids.

Said biocatalytic steps may be coupled to several other preceding (upstrean) or successive (downstream) enzymatic steps and allow the provision of a biocatalytic multistep process for the fully enzymatic synthesis of numerous valuable complex terpene molecules from their respective precursors.

The subsequent scheme illustrates two particular embodiments of two alternative pathways (“Enal cleaving polypeptide pathway” and “BMVO pathway)” of the present invention allowing the degradation of the labdane aldehyde copalal to manooloxy, which pathways are explained in more detail in the subsequent sections of the present specification. The scheme also illustrates the degradation of manooloxy to gamma-ambrol by applying a further BMVO-based degradation step.

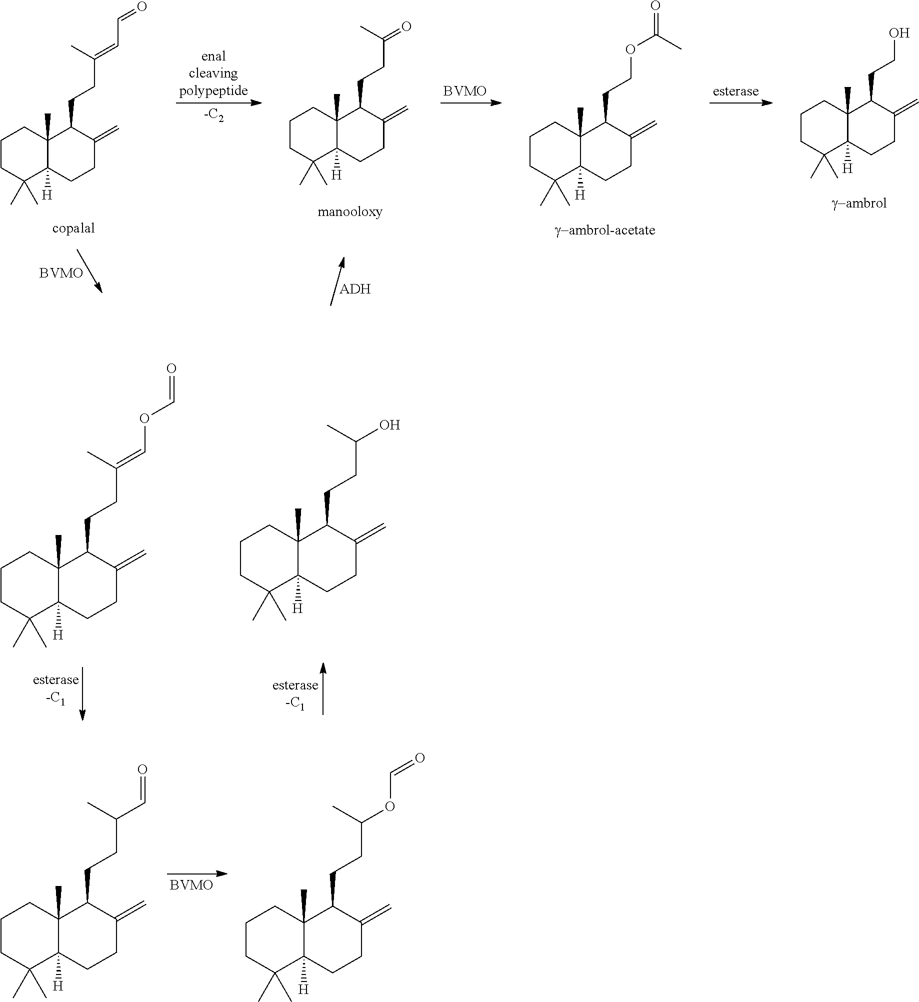

In full analogy to said exemplified reaction sequences this basic biosynthetic strategy my be applied to any other isomer of copalol or to any other labdane-type aldehyde in order to provide structurally related isomers of manooloxy, gamma-ambryl acetate or gamma-ambrol.

It also may be applied to structurally different mono-cyclic or non-cyclic carbonyl compounds as herein below specified in more detail.

### DESCRIPTION OF THE DRAWINGS

FIG. 1. Schematic representation of the chromosomal integration of the genes encoding for mevalonate pathway enzymes and organization of the two synthetic gene operons, mvaK1, a gene encoding a mevalonate kinase from S. pneumoniae; mvaD, a gene encoding a phosphomevalonate decarboxylase from S. pneumoniae: mvaK2, a gene encoding a phosphomevalonate kinase from S. pneumoniae; fni a gene encoding an isopentenyl diphosphate isomerase from S. pneumoniae; mvaA, a gene encoding an HMG-COA synthase from S, aureus; mvaS a genes encoding an HMG-COA reductase from S, aureus; atoB a gene encoding an acetoacetyl-CoA thiolase from E. coli; ERG20, a gene encoding an FPP synthase from S. cerevisiae.

FIG. 2. Conversion of manooloxy to gamma-ambryl acetate using BVMOs in an whole-cells bioconversion assay. GC-MS analysis of the products formed during the bioconversion of manooloxy by different BVMOs: SCH23-BVMO1, SCH24-BVMO1, SCH46-BVMO1. The upper chromatogram shows the GC-MS analysis of manooloxy. The lower chromatogram shows the GC-MS analysis of a bioconversion using control cells not expressing a recombinant BVMO.

FIG. 3. Conversion of copalal using BVMOs in whole-cells bioconversion assays. GC-MS analysis of the products formed (compounds 3a. 3b, 4a, 4b as described in the experimental part) during the bioconversion of cis-copalal and trans-copalal by different BVMOs: SCH23-BVMO1, SCH24-BVMO1, SCH46-BVMO1. The upper chromatogram shows the GC-MS analysis of a bioconversion using control cells not expressing a recombinant BVMO.

FIG. 4. Kinetic of the conversion of copalal using SCH23-BVMO1 in whole-cells bioconversion assays. GC-MS analysis of the products (compounds 1a, 1b, 3a, 3b, 4a, 4b as described in the experimental part) formed during the bioconversion of cis-copalal and trans-copalal by SCH23-BVMO1 after 0, 18 and 42 hours of incubation.

FIG. 5. In vitro conversion of manooloxy using BVMOs. GC-MS analysis of the conversion of manooloxy by SCH23-BVMO1 and SCH24-BVMO1 showing the formation of gamma-ambrol acetate. The upper chromatogram shows the GC-MS analysis of a conversion using control protein without recombinant BVMO.

FIG. 6. In vitro conversion of manooloxy using BVMOs and esterases. GC-MS analysis of the conversion of manooloxy by SCH23-BVMO1, SCH23-EST and the combination of SCH23-BVMO1 and SCH23-EST showing the formation of gamma-ambrol. The upper chromatogram shows the GC-MS analysis of a conversion using control protein without recombinant enzymes.

FIG. 7. In vitro conversion of manooloxy using BVMOs and esterases. GC-MS analysis of the conversion of manooloxy by SCH24-BVMO1, SCH24-EST and the combination of SCH24-BVMO1 and SCH24-EST showing the formation of gamma-ambrol. The upper chromatogram shows the GC-MS analysis of a conversion using control protein without recombinant enzymes.

FIG. 8. In vitro conversion of compounds 4a and 4b to compounds 5a and 5b using esterases. GC-MS analysis of the in-vitro conversion of compounds 4a and 4b by SCH23-EST1, SCH24-EST1 and SCH25-EST1 showing the formation of compounds 5a and 5b.

FIG. 9. In vitro conversion of copalal to compounds 5a and 5b using SCH23-BVMO1 and esterases. GCMS analysis of the in-vitro conversion of cis-copalal and trans-copalal by SCH23-BVMOs in combination with SCH23-EST1. SCH24-EST1 and SCH25-EST1 showing the formation of compounds 5a and 5b. The peak labelled with an * and at retention time of 11.95 minutes correspond to gamma-ambryl acetate: the observation of this compound in samples incubated with the BVMO alone is due to presence of small amounts of manooloxy in the mixture of copalal used in these assay.

FIG. 10. In vitro conversion of copalal to compounds 5a and 5b using SCH24-BVMO1 and esterases. GCMS analysis of the in-vitro conversion of cis-copalal and trans-copalal by SCH23-BVMOs in combination with SCH23-EST1 and SCH25-EST1 showing the formation of compounds 5a and 5b. The peak labelled with an * at retention time of 11.95 minutes correspond to gamma-ambryl acetate: the observation of this compound in samples incubated with the BVMO alone is due to presence of small amounts of manooloxy in the mixture of copalal used in these assay.

FIG. 11. Biochemical production of the 14,15-dinor-labdane compounds 5a and 5b and biosynthetic intermediates in engineered bacteria cells expression a BVMO and an esterase. The upper chromatogram shows the GC-MS analysis of compounds produced by E coli cells transformed with the pJ401-CPAL-1 plasmid allowing the expression of enzymes of a copalal biosynthetic pathway. The following chromatograms show the GC-MS analysis of cells further transformed with a second plasmid carrying nucleotide sequences encoding for a BVMO enzyme or a BVMO enzyme together with an esterase.

FIG. 12. GC-MS analysis of the products of the biotransformation of compounds 5a and 5b by E coli cells expressing various alcohol dehydrogenases. The upper chromatogram shows the GC-MS analysis of a bioconversion using control cells not expressing a recombinant alcohol dehydrogenase. The following chromatograms show the GC-MS analysis of a conversion using cells expressing the recombinant RrhSecADH, SCH80-00043. SCH80-0) 4254. SCH80-06135 or SCH80-06582 protein.

FIG. 13. Biochemical production of gamma-ambryl acetate and biosynthetic intermediates in engineered bacteria cells expression a BVMO, an esterase and an alcohol dehydrogenase. The upper chromatogram shows the GC-MS analysis of the compounds produced by E coli cells transformed with the pJ401-CPAL-1 plasmid allowing the expression of the enzymes of a copalal biosynthetic pathway. The middle chromatogram show the GC-MS analysis of cells further transformed with a second plasmid carrying nucleotide sequences encoding for a SCH-BVMO1 and SCH24-EST. The bottom chromatogram show the GC-MS analysis of cells transformed with pJ401-CPAL-1 and with the plasmid pJ423-secADH-23BVMO-EST allowing the expression of the RrhSecADH, SCH23-BVMO1 and SCH23-EST proteins.

FIG. 14. A) GC-MS analysis of terpenes and derivatives produced using the modified S. cerevisiae strains expressing the GGPP synthase carG, the CPP synthase SmCPS2, the CPP phosphatase TalVeTPP and either SCH23-ADH1, SCH23-BVMO1, SCH23-EST1 and SCH23-ADH2 (YST120) w/plasmid) or SCH24-ADH1a, SCH24-BVMO1, SCH24-EST1 and SCH24-ADH2a (YST121 w/plasmid). The control strain was YST075 expressing only the copalol biosynthetic pathway. B) GC-MS analysis of the region where farnesal was identified, the farnesal mass spectrum is shown. C) GC-MS analysis of the region where manooloxy was identified, the manooloxy mass spectrum is shown.

FIG. 15. GC-MS analysis of Manooloxy produced using the modified S. cerevisiae strains expressing the GGPP synthase carG, the CPP synthase SmCPS2, the CPP phosphatase TalVeTPP and either SCH23-ADH1, SCH23-BVMO1 and SCH23-EST1 (YST177) or SCH24-ADH1a, SCH24-BVMO1 and SCH24-EST1 (YST178). The control strain was YST075 expressing only the copalol biosynthetic pathway. The manooloxy mass spectrum is shown.

FIG. 16. GC-MS analysis of diterpenes and derivatives produced using E coli cells expressing a CPP synthase, a phosphatase, an alcohol dehydrogenase and/or SCH94-3944. The upper chromatogram shows the diterpene region the GC-MS analysis of compounds produced by E coli cells transformed with the pJ401-CPOL-4 plasmid allowing the expression of the enzymes of a copalol biosynthetic pathway. The following chromatograms shows the GC-MS analysis of the compounds produced by the same E coli cells further transformed with the plasmids pJ423-SCH94-3945, pJ423-SCH94-3944 or pJ423-SCH94-3944-3945 allowing the expression of SCH94-3945, SCH94-3944 or the combination of SCH94-3944 and SCH94-3945.

FIG. 17. GC-MS analysis of sesquiterpene and derivatives produced using E coli cells expressing a phosphatase, an alcohol dehydrogenase and SCH94-3944. The upper chromatogram shows the GC-MS analysis of the compounds produced by E coli cells transformed with the pJ401-FAL-1 plasmid allowing the expression of the enzymes of a farnesal biosynthetic pathway. The lower chromatograms shows the GC-MS analysis of the compounds produced by the same E coli cells further transformed with the plasmids pJ423-SCH94-3944 allowing the expression of the SCH94-3944 protein.

FIG. 18. GC-MS analysis of the products of the biotransformation of citral, citronelal and (F)-2-dodecanal by E coli cells expressing SCH94-3944. For each compounds the GC-MS analysis of the transformation using control E. coli cells and cells transformed to express the SCH94-3944 protein are show.

FIG. 19. GC-MS analysis of the sesquiterpenes and diterpenes produced using E coli cells expressing a CPP synthase, a phosphatase, an alcohol dehydrogenase. The chromatogram shows the GC-MS analysis of compounds produced by E coli cells transformed with the pJ401-CPAL-1 plasmid allowing the expression of the enzymes of a copalal biosynthetic pathway.

FIG. 20. GC-MS analysis of diterpenes and derivatives produced using E coli cells expressing a CPP synthase, a phosphatase, an alcohol dehydrogenase and SCH80-05241. SCH94-3944. PdigitDUF4334, PitalDUF4334-1 or AspWeDUF4334. The upper chromatogram shows the diterpene region in the GC-MS analysis of the compounds produced by E coli DP1205 cells transformed with the pJ401-CPAL-1 plasmid allowing the expression of the enzymes of a copalal biosynthetic pathway. The following chromatograms shows the GC-MS analysis of the compounds produced by the same E coli cells further transformed with a second plasmid expressing the SCH80-05241. SCH94-3944. PdigitDUF4334. PitalDUF4334-1 or AspWeDUF4334 recombinant proteins.

FIG. 21. GC-MS analysis of diterpenes and derivatives produced using E coli cells expressing a CPP synthase, a phosphatase, an alcohol dehydrogenase and CnecaDUF4334. Rins-DUF4334. RhoagDUF4334-2. RhoagDUF4334-3. RhoagDUF4334-4. CgatDUF4334. GclavDUF4334. TcurvaDUF4334 or PprotDUF4334. The upper chromatogram shows the diterpene region of a GC-MS analysis of the compounds produced by E coli DP1205 cells transformed with the pJ401-CPAL-1 plasmid allowing the expression of the enzymes of a copalal biosynthetic pathway. The following chromatograms shows the GC-MS analysis of the compounds produced by the same E coli cells further transformed with a second plasmid expressing the CnecaDUF4334. Rins-DUF4334. RhoagDUF4334-2. RhoagDUF4334-3. RhoagDUF4334-4. CgatDUF4334, GclavDUF4334. TcurvaDUF4334 or PprotDUF4334 recombinant proteins.

FIG. 22. GC-MS analysis of sesquiterpenes and derivatives produced using E coli cells expressing a phosphatase, an alcohol dehydrogenase and SCH80-05241. SCH94-3944. PdigitDUF4334. PitalDUF4334-1 or AspWeDUF4334. The upper chromatogram shows the sesquiterpene region in the GC-MS analysis of the compounds produced by E coli DP1205 cells transformed with the pJ401-CPAL-1 plasmid allowing the expression of the enzymes of a copalal biosynthetic pathway. The following chromatograms shows the GC-MS analysis of the compounds produced by the same E. coli cells further transformed with a second plasmid expressing the SCH80-05241. SCH94-3944. PdigitDUF4334, PitalDUF4334-1 or AspWeDUF4334 recombinant proteins.

FIG. 23. GC-MS analysis of sesquiterpenes and derivatives produced using E coli cells expressing a phosphatase, an alcohol dehydrogenase and CnecaDUF4334. Rins-DUF4334. RhoagDUF4334-2. RhoagDUF4334-3. RhoagDUF4334-4. CgatDUF4334. GclavDUF4334. TcurvaDUF4334 or PprotDUF4334. The upper chromatogram shows the sesquiterpene region of the GC-MS analysis of the compounds produced by E coli cells transformed with the pJ401-CPAL-1 plasmid allowing the expression of the enzymes of a copalal biosynthetic pathway. The following chromatograms shows the GC-MS analysis of the compounds produced by the same E coli cells further transformed with a second plasmid expressing the CnecaDUF4334. Rins-DUF4334. RhoagDUF4334-2. RhoagDUF4334-3. RhoagDUF4334-4. CgatDUF4334, GclavDUF4334. TcurvaDUF4334 or PprotDUF4334 recombinant proteins.

FIG. 24. Alignment and conserved amino acids of GXWXG and DUF4334 domain containing proteins catalazing the enzymatic enal-cleavage. The boxes show the predicted localization of the respective protein family domains. The depicted sequence listings correspond to the following: SCH94-3944 is set forth in SEQ ID NO:34, SCH80-05241 is set forth in SEQ ID NO:38. Pdigit7033 is set forth in SEQ ID NO: 42. PitalDUF4334-1 is set forth in SEQ ID NO: 46. AspWeDUF4334 is set forth in SEQ ID NO: 49. RhoagDUF4334-2 is set forth in SEQ ID NO: 53. RhoagDUF4334-3 is set forth in SEQ ID NO: 56. RhoagDUF4334-4 is set forth in SEQ ID NO: 59. ChecaDUF4334 is set forth in SEQ ID NO: 62. Rins-DUF4334 is set forth in SEQ ID NO: 69. CgatDUF4334 is set forth in SEQ ID NO: 72. GclavDUF4334 is set forth in SEQ ID NO: 75. TcurvaDUF4334 is set forth in SEQ ID NO:81, and PprotDUF4334 is set forth in SEQ ID NO: 87.

FIG. 25. Farnesal and copalal conversion activities by single amino acid variants of SCH94-3944. The activities are presented as the total amount of manooloxy and geranylacetone produced expressed in percentages relative to the wild type enzyme activities.

FIG. 26. GC-MS analysis of the biochemical production of manooloxy and gamma-ambryl acetate by E. coli cells expressing a CPP synthase, a phosphatase, an alcohol dehydrogenase, an enal cleaving enzyme and a BVMO. The upper chromatogram shows the diterpene region of the GC-MS analysis of the compounds produced by E coli DP1205 cells transformed with the pJ401-CPAL-1 plasmid allowing the expression of the enzymes of a copalal biosynthetic pathway. The following chromatograms shows the GC-MS analysis of the compounds produced by the same E coli cells further transformed with a second plasmid expressing the AspWeBVMO. SCH94-3944. SCH94-3944 together with AspWeBVMO. SCH94-3944 together with SCH23-BVMO1. SCH94-3944 together with SCH24-BVMO1, and SCH94-3944 together with SCH46-BVMO1.

FIG. 27. GC-MS analysis of terpenes and derivatives produced using the modified S. cerevisiae strains expressing the GGPP synthase carG, the CPP synthase SmCPS2, the CPP phosphatase TalVeTPP, the alcohol dehydrogenase SCH23-ADH1 and either AspWeDUF4334 (YST184). CnecaDUF4334 (YST185). Pdigit7033 (YST186). SCH94-3944 (YST187) or SCH80-05241 (YST188).

FIG. 28A) Percentages of identified terpenes produced by YST184. YST185. YST186. YST187 and YST188. B) Total amount of identified terpenes (SumT) produced by YST184. YST185. YST186. YST187 and YST188 with respect to the amount of identified terpenes in control (SumT-C). The control strain was YST075 expressing the copalol biosynthetic pathway.

FIG. 29. GC-MS analysis of terpenes and derivatives produced using the modified S. cerevisiae strains expressing the GGPP synthase carG, the CPP synthase SmCPS2, the CPP phosphatase TalVeTPP, the alcohol dehydrogenase SCH23-ADH, the enal-cleaving polypeptide AspWeDUF4334 and either SCH23-BVMO1 (YST190). SCH24-BVMO1 (YST191) or AspWeBVMO (YST192).

FIG. 30. A) Total amount of identified terpenes (SumT) produced by YST190. YST191 and YST192 with respect to the amount of identified terpenes in YST184 (SumT-C). B) Percentages of identified terpenes produced by YST190, YST191 and YST192.

FIG. 31. GC-MS analysis of the diterpene and diterpene derivatives produce using E. coli cells expressing a LPP synthase, a phosphatase, an alcohol dehydrogenase and enal-cleaving polypeptide. The upper chromatogram shows the GC-MS analysis of the compounds produced by E. coli DP1205 cells transformed with the pJ401-LOH-2 vector allowing the expression of the enzymes of a labdendiol biosynthetic pathway. The following chromatograms shows the GC-MS analysis of the compounds produced by the same E. coli cells further transformed with a second plasmid expressing the AzeTolADH1 alcohol dehydrogenase or the SCH94-3945 alcohol dehydrogenase together with the SCH94-3944 enal-cleaving polypeptide.

FIG. 32. Alignment and conserved amino acids of FMO-like domain containing proteins with BVMO activity. The boxes show the predicted localization of the respective protein family domains. The depicted sequence listings correspond to the following: SCH23-BVMO1 is set forth in SEQ ID NO:2, SCH24-BVMO1 is set forth in SEQ ID NO:6, SCH25-BVMO1 is set forth in SEQ ID NO:10, SCH46-BVMO1 is set forth in SEQ ID NO:13, and AspWeBVMO is set forth in SEQ ID NO: 16.

FIG. 33. GC-MS/FID analysis of terpenes and derivatives produced using the modified S. cerevisiae strains expressing the bifunctional PvCPS, the CPP phosphatase TalVeTPP, the alcohol dehydrogenase SCH23-ADH, the enal-cleaving polypeptide AspWeDUF4334, the Baeyer-Villiger monooxygenase SCH23-BVMO1 and either the esterase SCH23-EST (YST257) or the esterase SCH24-EST (YST258).

FIG. 34. GC-MS analysis of the biochemical production of gamma-ambrol by E. coli cells expressing a CPP synthase, a phosphatase, an alcohol dehydrogenase, an enal-cleaving enzyme, a BVMO and an esterase. A. GC-MS analysis of the compounds produced by E coli DP1205 cells transformed with the pJ401-Mnoxy plasmid allowing the expression of the enzymes of a manooloxy biosynthetic pathway. B. GC-MS analysis of the compounds produced by the same E. coli cells further expressing the a BVMO (SCH24-BVMO). C. GC-MS analysis of the compounds produced by the same E. coli cells further expressing the a BVMO (SCH24-BVMO) and an esterase (SCH24-EST).

### ABBREVIATIONS USED

ADH alcohol dehydrogenase
BVMO Baeyer-Villiger Monooxygenase
bp base pair
kb kilo base
CPP copalyl diphosphate
CPS copalyl diphosphate synthase
DNA deoxyribonucleic acid
cDNA complementary DNA
DMAPP dimethylallyl diphosphate
DTT dithiothreitol
FMO Flavin Monooxygenase
FPP farnesyl diphosphate
GPP geranyldiphosphate
GGPP geranylgeranyl diphosphate
GGPS geranylgeranyl diphosphate synthase
GC gas chromatograph
IPP isopentenyl diphosphate
LPP labdendiol diphosphate
LPS labdendiol diphosphate synthase
MS mass spectrometer/mass spectrometry
MVA mevalonic acid
PP diphosphate, pyrophosphate
PCR polymerase chain reaction
RNA ribonucleic acid
mRNA messenger ribonucleic acid
miRNA micro RNA
siRNA small interfering RNA
IRNA ribosomal RNA
tRNA transfer RNA
TPP terpenyl diphosphate

### Definitions

### a) General Terms

For the descriptions herein and the appended claims, the use of “or” means “and/or” unless stated otherwise. Similarly, “comprise”, “comprises”, “comprising”, “include”, “includes”, and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of”.

The terms “purified”, “substantially purified”, and “isolated” as used herein refer to the state of being free of other, dissimilar compounds with which a compound of the invention is normally associated in its natural state, so that the “purified”, “substantially purified”, and “isolated” subject comprises at least 0.5%, 1%, 5%, 10%, or 20%, or at least 50% or 75% of the mass, by weight, of a given sample. In one embodiment, these terms refer to the compound of the invention comprising at least 95, 96, 97, 98, 99 or 100%, of the mass, by weight, of a given sample. As used herein, the terms “purified,” “substantially purified,” and “isolated” when referring to a nucleic acid or protein, or nucleic acids or proteins, also refers to a state of purification or concentration different than that which occurs naturally, for example in an prokaryotic or eukaryotic environment, like, for example in a bacterial or fungal cell, or in the mammalian organism, especially human body. Any degree of purification or concentration greater than that which occurs naturally, including (1) the purification from other associated structures or compounds or (2) the association with structures or compounds to which it is not normally associated in said prokaryotic or eukaryotic environment, are within the meaning of “isolated”. The nucleic acid or protein or classes of nucleic acids or proteins, described herein, may be isolated, or otherwise associated with structures or compounds to which they are not normally associated in nature, according to a variety of methods and processes known to those of skill in the art.

The term “about” indicates a potential variation of ±25% of the stated value, in particular ±15%, ±10%, more particularly ±5%, ±2% or ±1%.

The term “substantially” describes a range of values of from about 80 to 100%, such as, for example, 85-99.9%, in particular 90 to 99.9%, more particularly 95 to 99.9%, or 98 to 99.9% and especially 99 to 99.9%.

“Predominantly” refers to a proportion in the range of above 50%, as for example in the range of 51 to 100%, particularly in the range of 75 to 99.9%, more particularly 85 to 98.5%, like 95 to 99%.

A “main product” in the context of the present invention designates a single compound or a group of at least 2 compounds, like 2, 3, 4, 5 or more, particularly 2 or 3 compounds, which single compound or group of compounds is “predominantly” prepared by a reaction as described herein, and is contained in said reaction in a predominant proportion based on the total amount of the constituents of the product formed by said reaction. Said proportion may be a molar proportion, a weight proportion or, preferably based on chromatographic analytics, an area proportion calculated from the corresponding chromatogram of the reaction products.

A “side product” in the context of the present invention designates a single compound or a group of at least 2 compounds, like 2, 3, 4, 5 or more, particularly 2 or 3 compounds, which single compound or group of compounds is not “predominantly” prepared by a reaction as described herein.

Because of the reversibility of enzymatic reactions, the present invention relates, unless otherwise stated, to the enzymatic or biocatalytic reactions described herein in both directions of reaction.

“Functional mutants” of herein described polypeptides include the “functional equivalents” of such polypeptides as defined below.

The term “stereoisomers” includes conformational isomers and in particular configuration isomers.

Included in general are, according to the invention, all “stereoisomeric forms” of the compounds described herein, such as “constitutional isomers” and “stereoisomers”.

“Stereoisomeric forms” encompass in particular, “stereoisomers” and mixtures thereof, e.g. configuration isomers (optical isomers), such as enantiomers, or geometric isomers (diastereomers), such as E- and Z-isomers, and combinations thereof. If one or more asymmetric centers are present in one molecule, the invention encompasses all combinations of different conformations of these asymmetry centers, e.g. enantiomeric pairs.

“Stereoselectivity” describes the ability to produce a particular stereoisomer of a compound in a stereoisomerically pure form or to specifically convert a particular stereoisomer in an enzyme catalyzed method as described herein out of a plurality of stereoisomers. More specifically, this means that a product of the invention is enriched with respect to a specific stereoisomer, or an educt may be depleted with respect to a particular stereoisomer. This may be quantified via the purity % ee-parameter calculated according to the formula:

$$\% ee = [ {X}_{A} - {X}_{B} ] / [ {X}_{A} + {X}_{B} ] * 100 ,$$

wherein X_{A} and X_{B} represent the molar ratio (Molenbruch) of the stereoisomers A and B.

The terms “selectively converting” or “increasing the selectivity” in general means that a particular stereoisomeric form, as for example the E-form, of an unsaturated hydrocarbon, is converted in a higher proportion or amount (compared on a molar basis) than the corresponding other stereoisomeric form, as for example Z-form, either during the entire course of said reaction (i.e. between initiation and termination of the reaction), at a certain point of time of said reaction, or during an “interval” of said reaction. In particular, said selectivity may be observed during an “interval” corresponding 1 to 99%, 2 to 95%, 3 to 90%, 5 to 85%, 10 to 80%, 15 to 75%, 20 to 70%, 25 to 65%, 30 to 60%, or 40 to 50% conversion of the initial amount of the substrate. Said higher proportion or amount may, for example, be expressed in terms of:
a higher maximum yield of an isomer observed during the entire course of the reaction or said interval thereof;
a higher relative amount of an isomer at a defined % degree of conversion value of the substrate; and/or
an identical relative amount of an isomer at a higher % degree of conversion value;
each of which preferably being observed relative to a reference method, said reference method being performed under otherwise identical conditions with known chemical or biochemical means.

Generally also comprised in accordance with the invention are all “isomeric forms” of the compounds described herein, such as constitutional isomers and in particular stereoisomers and mixtures of these, such as, for example, optical isomers or geometric isomers, such as E- and Z-isomers, and combinations of these. If several centers of asymmetry are present in a molecule, then the invention comprises all combinations of different conformations of these centers of asymmetry, such as, for example, pairs of enantiomers, or any mixtures of stereoisomeric forms.

“Yield” and/or the “conversion rate” of a reaction according to the invention is determined over a defined period of, for example, 4, 6, 8, 10, 12, 16, 20, 24, 36 or 48 hours, in which the reaction takes place. In particular, the reaction is carried out under precisely defined conditions, for example at “standard conditions” as herein defined.

The different yield parameters (“Yield” or Yps: “Specific Productivity Yield”; or Space-Time-Yield (STY)) are well known in the art and are determined as described in the literature.

“Yield” and “Yps” (each expressed in mass of product produced/mass of material consumed) are herein used as synonyms.

The specific productivity-yield describes the amount of a product that is produced per h and L fermentation broth per g of biomass. The amount of wet cell weight stated as WCW describes the quantity of biologically active microorganism in a biochemical reaction. The value is given as g product per g WCW per h (i.e. g/gWCW⁻¹ h⁻¹). Alternatively, the quantity of biomass can also be expressed as the amount of dry cell weight stated as DCW. Furthermore, the biomass concentration can be more easily determined by measuring the optical density at 600 nm (OD₆₀₀) and by using an experimentally determined correlation factor for estimating the corresponding wet cell or dry cell weight, respectively.

If the present disclosure refers to features, parameters and ranges thereof of different degree of preference (including general, not explicitly preferred features, parameters and ranges thereof) then, unless otherwise stated, any combination of two or more of such features, parameters and ranges thereof, irrespective of their respective degree of preference, is encompassed by the disclosure of the present description.

### b) Biochemical Terms

The term “domain” refers to a set of amino acids or a partial sequence of amino acids residues conserved at specific positions along an alignment of sequences of evolutionarily related proteins. While amino acids at other positions can vary between protein homologues, amino acids that are highly conserved at specific positions of such domain indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or consensus sequence” or “signature” refers to a short conserved region in the sequence of evolutionarily related proteins. Motifs are frequently highly conserved parts of domains, but may also include only part of the domain.

A “protein family” is defined as a group of proteins that share a common evolutionary origin reflected by their related functions, similarities in sequence, or similar primary, secondary or tertiary structure. Proteins within protein families are usually homologous and have similar structure of conserved functional domains and motifs.

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32: D134-D137, (2004)), or Pfam (Bateman et al., Nucleic Acids Research 30 (1): 276-280 (2002)). Domains or motifs may also be identified using routine techniques, such as by sequence alignment.

The term “Pfam” refers to a large collection of protein domains and protein families maintained by the Pfam Consortium and available at several sponsored world wide web sites, such as pfam.xfam.org//(European Molecular Biology Laboratory-European Bioinformatics Institute (EMBL_EBI). The latest release of Pfam is Pfam 32.0 (September 2018), based on the UniProt Reference Proteomes (El-Gebali S. et al, 2019, Nucleic Acids Res. 47, Database issue D427-D432). Pfam domains and families are identified using multiple sequence alignments and hidden Markov models (HMMs). Pfam-A family or domain assignments, are high quality assignments generated by a curated seed alignment using representative members of a protein family and profile hidden Markov models based on the seed alignment (Unless otherwise specified, matches of a queried protein to a Pfam domain or family are Pfam-A matches). All identified sequences belonging to the family are then used to automatically generate a full alignment for the family (Sonnhammer (1998) Nucleic Acids Research 26, 320-322: Bateman (2000) Nucleic Acids Research 26, 263-266; Bateman (2004) Nucleic Acids Research 32, Database Issue, D138-D141: Finn (2006) Nucleic Acids Research Database Issue 34, D247-251: Finn (2010) Nucleic Acids Research Database Issue 38, D211-222). By accessing the Pfam database, for example, using any of the above-reference websites, protein sequences can be queried against the HMMs using HMMER homology search software (e.g., HMMER2, HMMER3, or a higher version, hmmer.janelia.org/). Significant matches that identify a queried protein as being in a pfam family (or as having a particular Pfam domain) are those in which the bit score is greater than or equal to the gathering threshold for the Pfam domain. Expectation values (e-values) can also be used as a criterion for inclusion of a queried protein in a Pfam or for determining whether a queried protein has a particular Pfam domain, where low e-values, much less than 1.0, for example less than 0.1, or less.

The “E-value” (expectation value) is the number of hits that would be expected to have a score equal to or better than this value, by chance alone. This means that a good E-value which gives a confident prediction is much less than 1. E-values around 1 is what is expected by chance. Thus, the lower the E-value, the more specific the search for domains will be. Only positive numbers are allowed. (definition by Pfam))

A “precursor” molecule of a target compound as described herein is converted to said target compound, preferably through the enzymatic action of a suitable polypeptide performing at least one structural change on said precursor molecule. For example a “diphosphate precursor” (as for example a “terpenyl diphosphate precursor”) is converted to said target compound (as for example a terpene alcohol) via enzymatic removal of the diphosphate moiety, for example by removal of mono- or diphosphate groups by a phosphatase enzyme. For example a “non-cyclic precursor” (like a non-cyclic terpenyl precursor”) may be converted to the cyclic target molecule (like a cyclic terpene compound) through the action of a cyclase or synthase enzyme, irrespective of the particular enzymatic mechanism of such enzyme, in one or more steps.

The term “protein tyrosine phosphatase” represents a group of enzymes that are generally known to remove phosphate groups from phosphorylated tyrosine residues on proteins. A particular subgroup of said family as described herein are enzymes useful to dephosphory late phosphorylated terpene molecules.

A “terpene synthase” designates a polypeptide which converts a terpene precursor molecule to the respective terpene target molecule, like in particular a processed target terpene alcohol or terpene hydrocarbon. Non-limiting examples of such terpene precursor molecules are for example non-cyclic compounds, selected from farnesyl pyrophosphate (FPP), geranylgeranyl-pyrophosphate (GGPP), or a mixture of isopentenyl pyrophosphate (IPP) and dimethyl allyl pyrophosphate (DMAPP). In case the obtained terpene contains a diphosphate moiety the synthase is designated “terpenyl diphosphate synthase”

The terms “terpenyl diphosphate synthase” or “polypeptide having terpenyl diphosphate synthase activity” or “terpenyl diphosphate synthase protein” or “having the ability to produce terpenyl diphosphate” relate to a polypeptide capable of catalyzing the synthesis of a terpenyl diphosphate, in the form of any of its stereoisomers or a mixture thereof, starting from an acyclic terpene pyrophosphate, particularly GPP. FPP or GGPP or IPP together with DMAPP. The terpeny diphosphate may be the only product or may be part of a mixture of terpenyl phosphates. Said mixture may comprise terpenyl monophosphate and/or a terpene alcohol. The above definition also applies to the group of “bicyclic terpenyl diphosphate synthases”, which produce a bicyclic terpenyl diphosphate, like CPP or LPP. As example of such “terpenyl diphosphate synthase” enzymes there may be mentioned copalyl diphosphate synthase (CPS). Copalyl-diphosphate may be the only product or may be part of a mixture of copalyl phosphates. Said mixture may comprise copalyl-monophosphate and/or other terpenyl diphosphate. As another example of such “terpenyl diphosphate synthase” enzymes there may be mentioned and labdendiol diphosphate synthase (LPS). Labdendiol diphosphate may be the only product or may be part of a mixture of labdendiol phosphates. Said mixture may comprise labdendiol monophosphate and/or terpenyl diphosphate.

The terms “terpenyl diphosphate phosphatase” or “polypeptide having terpenyl diphosphate phosphatase activity” or “terpenyl diphosphate phosphatase protein” or “having the ability to produce terpene alcohol” relate to a polypeptide capable of catalyzing the removal (irrespective of a particular enzymatic mechanism) of a diphosphate moiety or monophosphate moieties, to form a dephosphorylated compound, in particular the corresponding alcohol compound of said terpenyl moiety. The terpene alcohol may be present in the product in any of its stereoisomers or as a mixture thereof. The terpene alcohol may be the only product or may be part of a mixture with other terpene compounds, as for example dephosphorylated analogs of the respective (for example non-cyclic) terpenyl diphosphate precursor of said terpenyl diphosphate. The above definition also applies to the group of “bicyclic terpenyl diphosphate phosphatase”, which produce a bicyclic terpene alcohol, like copalol or labdendiol.

As example of such “terpenyl diphosphate phosphatase” enzymes there may be mentioned copalyl diphosphate phosphatase (CPP phosphatase). Copalol may be the only product or may be part of a mixture with dephosphorylated precursors, like for example farnesol and/or geranylgeraniol; and/or side products resulting from enzymatic side activities in the reaction mixture, like esters or aldehydes of such alcohols or other cyclic or non-cyclic diterpenes. As another example of such “terpenyl diphosphate phosphatase” enzymes there may be mentioned and labdendiol diphosphate phosphatase (LPP phosphatase). Labdendiol may be the only product or may be part of a mixture with dephosphorylated precursors, like for example farnesol and/or geranylgeraniol; and/or side products resulting from enzymatic side activities in the reaction mixture, like esters or aldehydes of such alcohols or other cyclic or non-cyclic diterpenes.

An “enal-cleaving enzyme” or “enal-cleaving protein” or “enal-cleaving polypeptide” in the context of the present invention designates an “α,β-unsaturated aldehyde carbon-carbon double bond-cleaving enzyme, which also may be called a “α,β-unsaturated aldehyde C═C bond-cleaving enzyme” or “α,β-unsaturated aldehyde C═C-cleaving enzyme” or a “enal C═C-cleaving enzyme”. The enal-cleaving protein of the invention, based on protein domain organization, may also be described as a member of the ‘DUF4334 protein family” and/or as a member of the “GXWXG protein family”.

More particularly, an enal cleaving enzyme of the invention has the ability to cleave labdane-type carbonyl compounds, like labdane aldehydes, in particular copalal to the respective dinorlabdane carbonyl compound. “Baeyer-Villiger monooxygenases” (BVMOs) are flavoenzymes and belong to the class of refers to a polypeptide having oxidoreductase activity (EC 1.14.13.X). They catalyze the oxidation of linear, cyclic (aromatic or non-aromatic) aldehydes or ketones to the corresponding esters or lactones, highly similar to the chemical Baeyer-Villiger oxidation. During the enzymatic oxidation one atom of molecular oxygen is incorporated into a carbon-carbon bond of a non-activated carbonyl compound. The BVMOs require NADPH or NADH as cofactor or accept both. They also require molecular oxygen as co-substrate. More particularly, a BVMO of the invention has the ability to oxidize terpene-derived aldehydes or ketones, like for example labdane-type carbonyl compounds, like labdane aldehydes, in particular copalal and/or manooloxy to the respective carbonyl ester

An “esterase” refers to a polypeptide having hydrolase activity that splits esters into an acid and an alcohol in a chemical reaction with water (hydrolysis). Esterases in the context of the present invention are selected from the class of carboxylic ester hydrolases (EC 3.1.1.-), which splits off acyl groups, like acetyl or formyl groups, from the respective etser substrate. More particularly, an esterase of the invention has the ability to cleave labdane-type ester compounds, like gamma-ambryl-acetate, to form the respective labdane-type alcohol, like gamma-ambrol.

An “alcohol dehydrogenase” (ADH) in the context of the present invention refers to a polypeptide having the ability to oxidize an alcohol to the corresponding aldehyde in the presence of NAD⁺ or NADP⁺ as cofactor. Such enzymes are members of the E.C. families 1.1.1.1 (NAD dependent) or 1.1.1.2 (NADP′ dependent). More particularly, an ADH of the invention has the ability to oxidize labdane-type alkohols to the respective labdane-type carbonyl compounds (aldehydes or ketones), like copalol to copalal and/or labdendiol to the respective aldehyde or other labdane-type derivatives of copalol, labdendiol, for example the respective nor- or dinor-labdane derivatives of copalol or labdendiol. ADHs a sused herein may either be endogenously present in the respective biocatalytic process or may be exogenous.

“Enal-cleaving activity” is determined under “standard conditions” as described herein below: It can be determined using recombinant enal-cleaving polypeptide expressing host cells, disrupted enal-cleaving polypeptide expressing cells, fractions of these or enriched or purified enal-cleaving polypeptide, in a culture medium or reaction medium, preferably buffered, having a pH in the range of 6 to 11, preferably 7 to 9, at a temperature in the range of about 20 to 45° C., like about 25 to 40° C., preferably 25 to 32° C., and in the presence of a reference substrate, here in particular copalal, either added at an initial concentration in the range of 1 to 100 μM mg/ml, preferably 5 to 50 μM, in particular 30 to 40 μM, or endogenously produced by the host cell. The conversion reaction to form the respective cleavage product, like manooloxy is conducted from 10 min to 5 h, preferably about 1 to 2 h. The cleavage product may then be determined in conventional matter, for example after extraction with an organic solvent, like ethyl acetate.

“BVMO activity” is determined under “standard conditions” as described herein below: It can be determined using recombinant BVMO expressing host cells, disrupted BVMO expressing cells, fractions of these or enriched or purified BVMO enzyme, in a culture medium or reaction medium, preferably buffered, having a pH in the range of 6 to 11, preferably 7 to 9, at a temperature in the range of about 20 to 45° C. like about 25 to 40° C. preferably 25 to 32° C., and in the presence of a reference substrate, here in particular copalal and/or manooloxy, either added at an initial concentration in the range of 1 to 100 μM mg/ml, preferably 5 to 50 μM, in particular 30 to 40 μM, or endogenously produced by the host cell and in the presence of molecular oxygen. For in-vitro assays a cofactor selected from NADH and NADPH has to be added in a suitable easily to be determined concentration range of The conversion reaction to form the respective cleavage product, like the formyl esters 1a and/or 1b in the case of copalal or gamma-ambryl acetate in the case of manooloxy is conducted from 10 min to 5 h. preferably about 1 to 2 h. The oxidation product may then be determined in conventional matter, for example after extraction with an organic solvent, like ethyl acetate.

“Terpenyl diphosphate synthase activity” (like CPS or LPS activity) is determined under “standard conditions” as described herein below: They can be determined using recombinant terpenyl diphosphate synthase expressing host cells, disrupted terpenyl diphosphate synthase expressing cells, fractions of these or enriched or purified terpenyl diphosphate synthase enzyme, in a culture medium or reaction medium, preferably buffered, having a pH in the range of 6 to 11, preferably 7 to 9, at a temperature in the range of about 20 to 45° C. like about 25 to 40° C. preferably 25 to 32° C., and in the presence of a reference substrate, here in particular GGPP, either added at an initial concentration in the range of 1 to 100 μM mg/ml, preferably 5 to 50 μM, in particular 30 to 40 μM, or endogenously produced by the host cell. The conversion reaction to form a terpenyl diphosphate is conducted from 10 min to 5 h. preferably about 1 to 2 h. If no endogenous phosphatase is present, one or more exogenous phosphatases, for example an alkaline phosphatase, are added to the reaction mixture to convert the terpenyl diphosphate as formed by the synthase to the respective terpene alcohol. The terpene alcohol may then be determined in conventional matter, for example after extraction with an organic solvent, like ethyl acetate.

“Terpenyl diphosphate phosphatase activity” (like CPP or LPP phosphatase activity) is determined under “standard conditions” as described herein below: They can be determined using recombinant terpenyl diphosphate phosphatase expressing host cells, disrupted terpenyl diphosphate phosphatase expressing cells, fractions of these, or enriched or purified terpenyl diphosphate phosphatase enzyme, in a culture medium or reaction medium, preferably buffered, having a pH in the range of 6 to 11, preferably 7 to 9, at a temperature in the range of about 20 to 45° C., like about 25 to 40° C., preferably 25 to 32° C., and in the presence of a reference substrate, here for example CPP or LPP, either added at an initial concentration in the range of 1 to 100 μM mg/ml, preferably 5 to 50 μM, in particular 30 to 4 μM, or endogenously produced by the host cell. The conversion reaction to form a terpenyl diphosphate is conducted from 10 min to 5 h, preferably about 1 to 2 h. The terpene alcohol may then be determined in conventional matter, for example after extraction with an organic solvent, like ethyl acetate.

Particular examples of suitable standard conditions for each of the above-described enzyme activities may be taken from the Experimental Part below.

The terms “biological function,” “function”, “biological activity” or “activity” of a terpeyl synthase refer to the ability of a terpenyl diphosphate synthase as described herein to catalyze the formation of at least one terpenyl diphosphate from the corresponding precursor terpene.

The terms “biological function,” “function”, “biological activity” or “activity” of a terpenyl diphosphate phosphatase refer to the ability of the terpenyl diphosphate phosphatase as described herein to catalyze the removal of a diphosphate group from said terpenyl compound to form the corresponding terpene alcohol.

The “mevalonate pathway” also known as the “isoprenoid pathway” or “HMG-COA reductase pathway” is an essential metabolic pathway present in eukaryotes, archaea, and some bacteria. The mevalonate pathway begins with acetyl-CoA and produces two five-carbon building blocks called isopentenyl pyrophosphate (IPP) and dimethyl allyl pyrophosphate (DMAPP). Key enzymes are acetoacetyl-CoA thiolase (atoB), HMG-COA synthase (mvaS), HMG-COA reductase (mvaA), mevalonate kinase (MvaK1), phosphomevalonate kinase (MvaK2), a mevalonate diphosphate decarboxylase (MvaD), and an isopentenyl diphosphate isomerase (idi). Combining the mevalonate pathway with enzyme activity to generate the terpene precursors GPP, FPP or GGPP, like in particular FPP synthase (ERG20), allows the recombinant cellular production of terpenes.

As used herein, the term “host cell” or “transformed cell” refers to a cell (or organism) altered to harbor at least one nucleic acid molecule, for instance, a recombinant gene encoding a desired protein or nucleic acid sequence which upon transcription yields at least one functional polypeptide of the present invention, in particular a terpenyl diphosphate synthase protein or terpenyl diphosphate phosphatase enzyme as defined herein above. The host cell is particularly a bacterial cell, a fungal cell or a plant cell or plants. The host cell may contain a recombinant gene or several genes, as for example organized as an operon, which has been integrated into the nuclear or organelle genomes of the host cell. Alternatively, the host may contain the recombinant gene extra-chromosomally.

The term “organism” refers to any non-human multicellular or unicellular organism such as a plant, or a microorganism. Particularly, a micro-organism is a bacterium, a yeast, an algae or a fungus.

The term “plant” is used interchangeably to include plant cells including plant protoplasts, plant tissues, plant cell tissue cultures giving rise to regenerated plants, or parts of plants, or plant organs such as roots, stems, leaves, flowers, pollen, ovules, embryos, fruits and the like. Any plant can be used to carry out the methods of an embodiment herein.

A particular organism or cell is meant to be “capable of producing FPP” when it produces FPP naturally or when it does not produce FPP naturally but is transformed to produce FPP with a nucleic acid as described herein., Organisms or cells transformed to produce a higher amount of FPP than the naturally occurring organism or cell are also encompassed by the “organisms or cells capable of producing FPP”.

A particular organism or cell is meant to be “capable of producing GGPP” when it produces GGPP naturally or when it does not produce GGPP naturally but is transformed to produce GGPP with a nucleic acid as described herein. Organisms or cells transformed to produce a higher amount of GGPP than the naturally occurring organism or cell are also encompassed by the “organisms or cells capable of producing GGPP”.

A particular organism or cell is meant to be “capable of producing terpenyl diphosphate” when it produces a terpenyl diphosphate as defined herein naturally or when it does not produce said diphosphate naturally but is transformed to produce said diphosphate with a nucleic acid as described herein. Organisms or cells transformed to produce a higher amount of terpenyl diphosphate than the naturally occurring organism or cell are also encompassed by the “organisms or cells capable of producing a terpenyl diphosphate”.

A particular organism or cell is meant to be “capable of producing terpene alcohol” when it produces a terpene alcohol as defined herein naturally or when it does not produce said alcohol naturally but is transformed to produce said alcohol with a nucleic acid as described herein. Organisms or cells transformed to produce a higher amount of a terpene alcohol than the naturally occurring organism or cell are also encompassed by the “organisms or cells capable of producing a terpene alcohol”. The same applies to a particular organism “capable of producing labdane-type alcohol”.

A particular organism or cell is meant to be “capable of producing an ester” when it produces an ester as defined herein naturally or when it does not produce said ester naturally but is transformed to produce said ester with a nucleic acid as described herein. Organisms or cells transformed to produce a higher amount of ester than the naturally occurring organism or cell are also encompassed by the “organisms or cells capable of producing an ester”.

A particular organism or cell is meant to be “capable of producing a target product” when it produces a target product as defined herein (for example the esters, alcohol, or carbonyl compounds or more particularly the labdane type compounds) naturally or when it does not produce said target product naturally but is transformed to produce said target product with a nucleic acid as described herein. Organisms or cells transformed to produce a higher amount of target product than the naturally occurring organism or cell are also encompassed by the “organisms or cells capable of producing a target product”.

The term “fermentative production” or “fermentation” refers to the ability of a microorganism (assisted by enzyme activity contained in or generated by said microorganism) to produce a chemical compound in cell culture utilizing at least one carbon source added to the incubation.

The term “fermentation broth” is understood to mean a liquid, particularly aqueous or aqueous/organic solution which is based on a fermentative process and has not been worked up or has been worked up, for example, as described herein.

An “enzymatically catalyzed” or “biocatalytic” method means that said method is performed under the catalytic action of an enzyme, including enzyme mutants, as herein defined. Thus the method can either be performed in the presence of said enzyme in isolated (purified, enriched) or crude form or in the presence of a cellular system, in particular, natural or recombinant microbial cells containing said enzyme in active form, and having the ability to catalyze the conversion reaction as disclosed herein.

### c) Chemical Terms

The term “alpha, beta-unsaturated carbonyl” compound describes organic molecules containing an aldehyde or keto group of the general formula R^{a}R^{b}C═C(R^{c})—C—O, wherein the C═C bond may be of any stereoisomeric configuration and wherein residues Rₐ, R^{b} and R^{c} may be identical or different and may have the meanings as specified below for particular alpha, beta unsaturated carbonyl compounds.

A “labdane” compound in the context of the present invention will show the following basic structure of its carbon skeleton consisting of 20 carbon atoms. The depicted numbering of carbon atoms will be applied in order to further define certain positions within said carbon skeleton.

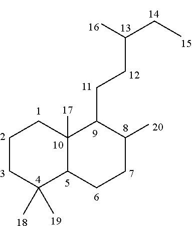

The term “labdane” encompasses any compounds of this basic C₂₀-structure, in any stereoisomeric form and encompassing any variant of this structure containing one or more unsaturated C—C bonds, in particular one or more C═C bonds, at any position, within the carbocyclic ring and/or the side chains. Also encompassed are variants thereof containing one or more substituents, as for example substituents selected from the group of —OH, ═O, —O—CO_R, wherein R may be straight chain or branched alkyl, in particular lower alkyl, more particularly C₁-C₄ aklyl, like methyl, ethyl, n- or i-propyl, or n-, i- or t-butyl; and —COOH at any of the indicated primary, secondary or tertiary C atoms.

A “labdane derived” compound of such “labdane” encompasses chemical compounds wherein the basic C₂₀-carbon skeleton is modified by deleting one or more carbon atoms. As examples there may be mentioned:
norlabdane (C₁₉-sceleton), dinorlabdane (C₁₈-sceleton), trinorlabdane (C₁₇-sceleton), and tetranorlabdane (C₁₆-sceleton). The position of the deleted carbon atom is indicated by stating the carbon number. For example, in a norlabdane, wherein the carbonate in position 15 is missing is designated “15-norlabdane”.

A “labdane derived” compound of such “labdane” also encompasses chemical compounds wherein the basic C₂₀-carbon skeleton is modified by inserting a hereoatom between two C-atoms of the labdane sceleoton. For example, insertion of an ether bridge between positions 14 and 15 converts the labdane to a norlabdane and particularly to a norlabdane ester.

Non-limiting examples of substituted labdanes or substituted labdane derived structures are given below:

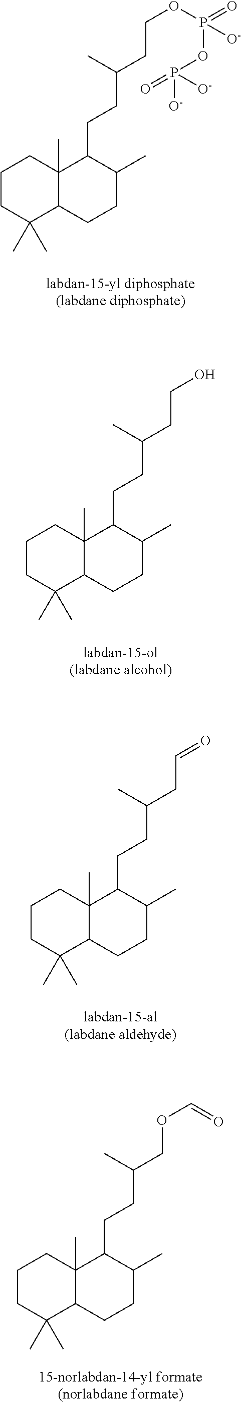 [SMILES: CC(CCOP(=O)([O-])OP(=O)([O-])[O-])CCC1C(C)CCC2C(C)(C)CCCC12C ; CC(CCO)CCC1C(C)CCC2C(C)(C)CCCC12C ; CC(CC=O)CCC1C(C)CCC2C(C)(C)CCCC12C ; CC(CCC1C(C)CCC2C(C)(C)CCCC12C)COC=O]

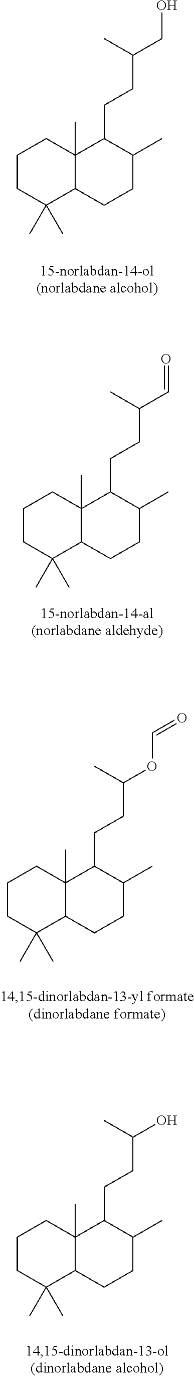 [SMILES: CC(CO)CCC1C(C)CCC2C(C)(C)CCCC12C ; CC(C=O)CCC1C(C)CCC2C(C)(C)CCCC12C ; CC(CCC1C(C)CCC2C(C)(C)CCCC12C)OC=O ; CC(O)CCC1C(C)CCC2C(C)(C)CCCC12C]

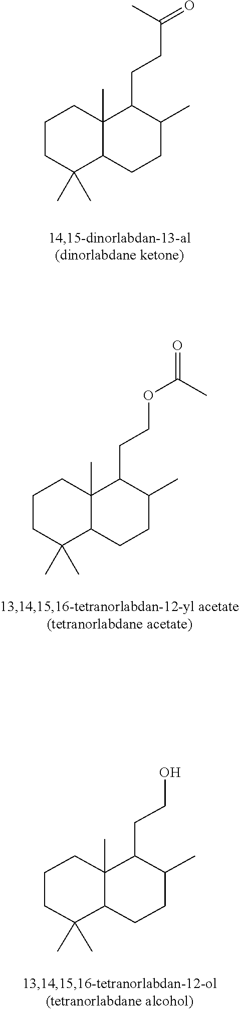 [SMILES: CC(=O)CCC1C(C)CCC2C(C)(C)CCCC12C ; CC(=O)OCCC1C(C)CCC2C(C)(C)CCCC12C ; CC1CCC2C(C)(C)CCCC2(C)C1CCO]

“Diphosphate” and “pyrophosphate” as used herein are synonyms.

“Terpenes” are a large and diverse class of organic compounds, produced by a variety of plants, particularly conifers, and by some insects. Terpenes are hydrocarbons. Although sometimes used interchangeably with “terpenes”, “terpenoids” or “isoprenoids” are modified terpenes as they contain additional functional groups, usually oxygen-containing.

“Terpenoids” (“isoprenoids”) are a large and diverse class of naturally occurring organic chemicals derived from terpenes. Although sometimes used interchangeably with the term “terpenes”, “terpenoids” contain additional functional groups, usually O-containing groups, like for example hydroxyl, carbonyl or carboxyl groups. Most are multicyclic structures with oxygen-containing functional groups. Unless stated otherwise, in the context of the present description the term “terpene” and the term “terpenoid” may be used interchangeably.

Terpenes (and terpenoids) may be classified by the number of isoprene units in the molecule: a prefix in the name indicates the number of terpene units needed to assemble the molecule. Hemiterpenes consist of a single isoprene unit. Monoterpenes consist of two isoprene units and have the molecular formula C₁₀H₁₆. Sesquiterpenes consist of three isoprene units and have the molecular formula C₁₅H₂₄. Diterpenes are composed of four isoprene units and have the molecular formula C₂₀H₃₂.

“Terpenyl” designates noncyclic and cyclic chemical hydrocarbyl residues which are derived from the C₅ building block isoprene and in particular contain one or more such building blocks.

“Cyclic terpene” or cyclic terpenyl” or “cyclic diterpene” or cyclic diterpenyl” relates to a terpene compound or terpenyl residue which comprises in its structure at lest on, as for example 1, 2, 3, 4 or 5 carbocyclic condensed and/or non-condensed rings, preferably two carbocyclic condensed rings.

“Bicyclic terpene” or bicyclic terpenyl” or “bicyclic diterpene” or bicyclic diterpenyl″ relates to a terpene compound or terpenyl residue which comprises in its structure two carbocyclic rings, preferably two carbocyclic condensed rings.

“Derivatives of terpenes” or “derivatives of terpenoids” in the context of the present invention in particular refer to such chemical compounds which are obtained from a terpene or terpenoid by chemical and/or enzymatic modification. More particularly, such derivatives encompass “hydrocarbon chain-degraded” derivatives.

A “hydrocarbon chain-degraded” terpene or terpenoid differs from the non-degraded precursor by a reduced number of carbon items of the precursor's carbon skeleton.

A “hydrocarbyl” residue is a chemical group which essentially is composed of carbon and hydrogen atoms and may be a non-cyclic, linear or branched, saturated or unsaturated moiety, or a cyclic saturated or unsaturated moiety, aromatic or non-aromatic moiety. A hydrocarbyl residue comprises 1 to 30, 1 to 25, 1 to 20, 1 to 15 or 1 to 10 or 1 to 5 carbon atoms in the case of a non-cyclic structure. It comprises 4 to 30, 4 to 25, 4 to 20, 4 to 15, 4 to 10 or in particular 4, 5, 6 or 7 carbon atoms in the case of a cyclic structure.

Said hydrocarbyl residues may be non-substituted or may carry at least one, like 1 to 5, preferably 0, 1 or 2 substituents.

Particular examples of such hydrocarbyl residues are noncyclic linear or branched alkyl or alkenyl residues as defined below; or mono- or polycyclic, in particular mono- or bicyclic, saturated or unsaturated, nonaromatic moieties, as for example found in cyclic (for example bicyclic) or noncyclic terpene type compound, and labdane type compounds as defined herein.

An “alkyl” residue represents linear or branched, saturated hydrocarbon residues. It comprises 1 to 30, 1 to 25, 1 to 20, 1 to 15 or 1 to 10 or 1 to 7, 1 to 6, 1 to 5, or 1 to 4 carbon atoms.

An “alkenyl” residue represents linear or branched, mono- or polyunsaturated hydrocarbon residues. It comprises 2 to 30, 2 to 25, 2 to 20, 2 to 15 or 2 to 10 or 2 to 7, 2 to 6, 2 to 5, or 2 to 4 carbon atoms. I may have up to 10, like 1, 2, 3, 4 or 5 C═C double bonds.

The term “lower alkyl” or “short chain alkyl” represents saturated, straight-chain or branched hydrocarbon radicals having 1 to 4, 1 to 5, 1 to 6, or 1 to 7, in particular 1 to 4 carbon atoms. As examples there may be mentioned: methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; and also n-heptyl, and the singly or multiply branched analogs thereof.

“Long-chain alkyl” represents, for example, saturated straight-chain or branched hydrocarbyl radicals having 8 to 30, for example 8 to 20 or 8 to 15, carbon atoms, such as octyl, nonyl, decyl, undecyl, dodecyl, tridecyl, tetradecyl, pentadecyl, hexadecyl, heptadecyl, octadecyl, nonadecyl, eicosyl, hencosyl, docosyl, tricosyl, tetracosyl, pentacosyl, hexacosyl, heptacosyl, octacosyl, nonacosyl, squalyl, constitutional isomers, especially singly or multiply branched isomers thereof.

“Long-chain alkenyl” represents the mono- or polyunsaturated analogues of the above mentioned “long-chain alkyl” groups,

“Short chain alkenyl” (or “lower alkenyl”) represents mono- or polyunsaturated, especially monounsaturated, straight-chain or branched hydrocarbon radicals having 2 to 4, 2 to 6, or 2 to 7 carbon atoms and one double bond in any position, e.g. C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,3-dimethyl-1-butenyl, 2,2-dimethyl-3-butenyl, 1,2-dimethyl-2-butenyl, 1,3-dimethyl-2-butenyl, 1,2-dimethyl-3-butenyl, 1,3-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

“Alkylene” represents straight-chain or singly or multiply branched hydrocarbon bridging groups having 1 to 10 carbon atoms, for example C₁-C₇-alkylene groups selected from —CH₂—, —(CH₂)₂—, —(CH₂)₃—, —(CH₂)₄—, —(CH₂)₂—CH(CH₃)—, —CH₂—CH(CH₃)—CH₂—, (CH₂)₄—, —(CH₂)₅—, —(CH₂)₆, —(CH₂)₇—, —CH(CH₃)—CH₂—CH₂—CH(CH₃)— or —CH(CH₃)—CH₂—CH₂—CH₂—CH(CH₃)—, and in particular C₁-C₄-alkylene groups selected from —CH₂—, —(CH₂)₂—, —(CH₂)₃—, —(CH₂)₄—, —(CH₂)₂—CH(CH₃)—, —CH₂—CH(CH₃)—CH₂—.

An “alkylidene” group represents a straight chain or branched hydrocarbon substituent linked via a double bond to the body of the molecule. It comprises 1 to 6 carbon atoms. As examples of such “C₁-C₆-alkylidenes” there may be mentioned methylidene (═CH₂) ethylidene, (═CH—CH₂), n-propylidene, n-butylidene, n-pentlyiden, n-hexylidene and the constitutional isomers thereof, as for example iso-propylidene.

An “alkenylidene” represents the mono-unsaturated analogue of the above mentioned alkylidenes with more than 2 carbon atoms and may be called “C₃-C₆-alkenylidenes”. n-propenylidene, n-butenylidene, n-pentenlyiden, and n-hexenylidene may be mentioned as examples.

The “substituent” of the above mentioned residues contains one hetero atom, like O or N. Preferably the substituents are independently selected from —OH, C═O, or —COOH. Most preferably said substituent is-OH.

A “mono- or polycyclic hydrocarbyl residue” comprise 1, 2 or 3 condensed (anellated) or non-condensed, optionally substituted, saturated or unsaturated hydrocarbon ring groups (or “carbocyclic” groups). Each cycle may comprise independently of each other 3 to 8, in particular 5 to 7, more particularly 6 ring carbon atoms. As examples of monocyclic residues there may be mentioned “cycloalkyl” groups which are carbocyclic radicals having 3 to 7 ring carbon atoms, such as cyclopropyl, cyclobutyl, cyclopentyl, cyclohexyl, cycloheptyl, and cyclooctyl; and the corresponding “cycloalkenyl” groups. Cycloalkenyl” (or “mono- or polyunsaturated cycloalkyl”) represents, in particular, monocyclic, mono- or polyunsaturated carbocyclic groups having 5 to 8, preferably up to 6, carbon ring members, for example monounsaturated cyclopentenyl, cyclohexenyl, cycloheptenyl and cyclooctenylradicals.

As examples of polycyclic residues there may be mentioned groups wherein 1, 2 or 3 of such cycloalkyl and/or cycloalkenyl are linked together, as for example anellated, in order to form a polycyclic cycloalkyl or cycloalkenyl ring. As non-limiting example the bicyclic decalinyl residue composed of two anellated 6-membered carbon rings may be mentioned.

The number of substituents in such mono- or polycyclic hydrocarbyl residues may vary from 1 to 10, in particular 1 to 5 substituents. Suitable substituents of such cyclic residues are selected from lower alkyl, lower alkenyl, alkylidene, alkenylidene, or residues containing one hetero atom, like O or N as for example —OH or —COOH. In particular the substituents are independently selected from —OH, —COOH, methyl and methylidene.

Unsaturated cyclic groups may contain 1 or more, as for example 1, 2 or 3 C═C bonds and are aromatic, or in particular nonaromatic.

The above-mentioned mono- or polycyclic saturated or unsaturated groups may also contain at least one, like 1, 2, 3 or 4 ring heteroatoms, such as O, N or S.

Overview of particular compound names and their structural formulae

| Structure | IUPAC name | other names |
|---|---|---|
| 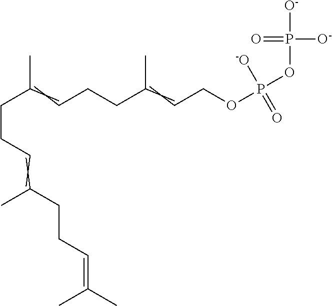 | [oxido(3,7,11,15- tetramethylhexadeca- 2,6,10,14- tetraenoxy)phosphoryl] phosphate | cis/trans- geranylgeranyl pyrophosphate, cis/trans-GPP |
| 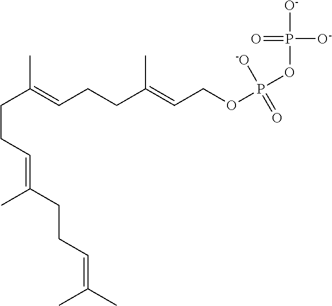 | [oxido-[(2E,6E,10E)- 3,7,11,15- tetramethylhexadeca- 2,6,10,14- tetraenoxy]phosphoryl] phosphate | (E,E,E)- geranylgeranyl pyrophosphate, (E,E,E)-GPP |

| | IUPAC | No. in | Labdane | |
|---|---|---|---|---|
| Structure | name | examples | nomenclature | Short names |
| 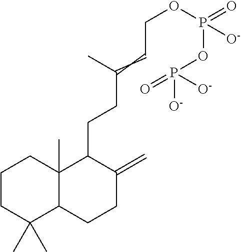 [SMILES: C=C1CCC2C(C)(C)CCCC2(C)C1CCC(C)=CCOP(=O)([O-])OP(=O)([O-])[O-]] | [[5-(5,5,8a- trimethyl-2- methylene-decalin- 1-yl]-3-methyl- pent-2-enoxy]- oxido-phosphoryl] phosphate | | labda-8(20), 13- dien-15-yl diphosphate | |
| 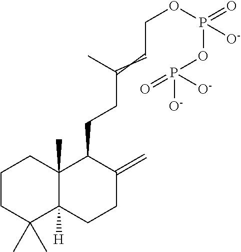 [SMILES: C=C1CC[C@H]2C(C)(C)CCC[C@]2(C)[C@H]1CCC(C)=CCOP(=O)([O-])OP(=O)([O-])[O-]] | [[5-[(1S,4aS,8aS)- 5,5,8a-trimethyl-2- methylene-decalin- 1-yl]-3-methyl- pent-2-enoxy]- oxido-phosphoryl] phosphate | | (5S,9S,10S)- labda-8(20), 13- dien-15-yl diphosphate | cis/trans-copalyl diphosphate, cis/trans-CPP |
| 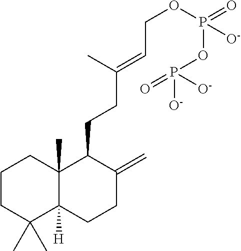 [SMILES: C=C1CC[C@H]2C(C)(C)CCC[C@]2(C)[C@H]1CC/C(C)=C/COP(=O)([O-])OP(=O)([O-])[O-]] | [[(E)-5-[(1S,4aS,8aS)- 5,5,8a-trimethyl-2- methylene-decalin- 1-yl]-3-methyl- pent-2-enoxy]- oxido-phosphoryl] phosphate | | (5S,9S,10S)-- (13E)-labda- 8(20), 13-dien-15- yl diphosphate | trans-copalyl diphosphate, Trans-CPP, CPP |
| 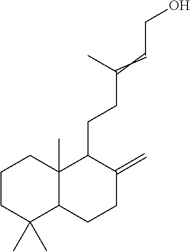 [SMILES: C=C1CCC2C(C)(C)CCCC2(C)C1CCC(C)=CCO] | 5-(5,5,8a-trimethyl- 2-methylene-decalin-(1-yl)- 3-methyl-pent-2-en-1-ol | | labda-8(20), 13-dien-15-ol | |
| 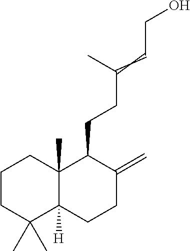 [SMILES: C=C1CC[C@H]2C(C)(C)CCC[C@]2(C)[C@H]1CCC(C)=CCO] | 5-[(1S,4aS,8aS)-5,5,8a- trimethyl-2-methylene- decalin-1-yl]-3-methyl- pent-2-en-1-ol | | (5S,9S,10S)- labda-8(20), 13-dien-15-ol | cis/trans-copalol |
| 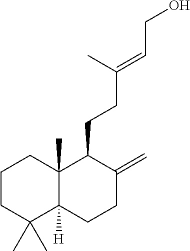 [SMILES: C=C1CC[C@H]2C(C)(C)CCC[C@]2(C)[C@H]1CC/C(C)=C/CO] | (E)-5-[(1S,4aS,8aS)-5,5,8a- trimethyl-2-methylene- decalin-1-yl]-3-methyl- pent-2-en-1-ol | | (5S,9S,10S)- (13E)-labda-8(20), 13-dien-15-ol | (+)-trans copalol |
| 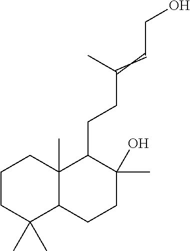 [SMILES: CC(=CCO)CCC1C(C)(O)CCC2C(C)(C)CCCC21C] | 1-(5-hydroxy-3- methyl-pent-3- enyl]-2,5,5,8a- tetramethyl- decalin-2-ol | | labd-13-en- 8,15-diol | |
| 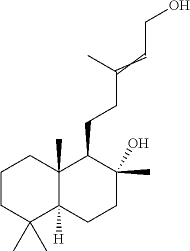 [SMILES: CC(=CCO)CC[C@@H]1[C@@]2(C)CCCC(C)(C)[C@@H]2CC[C@@]1(C)O] | (1R,2R,4aS,8aS)-1- (5-hydroxy-3- methyl-pent-3- enyl)-2,5,5,8a- tetramethyl- decalin-2-ol | | (5S,8R,9R,10S)- labd-13-en-8,15- diol | cis/trans- labdendiol |
| 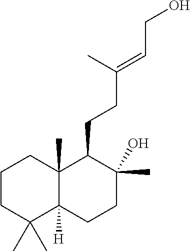 [SMILES: C/C(=C\CO)CC[C@@H]1[C@@]2(C)CCCC(C)(C)[C@@H]2CC[C@@]1(C)O] | (1R,2R,4aS,8aS)-1- [(E)-5-hydroxy-3- methyl-pent-3- enyl)-2,5,5,8a- tetramethyl- decalin-2-ol | | (5S,8R,9R,10S)- (13E)-5-labd-13 en-8,15-diol | trans- labdendiol |
| 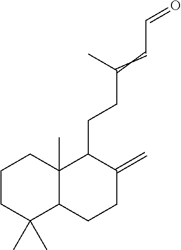 [SMILES: C=C1CCC2C(C)(C)CCCC2(C)C1CCC(C)=CC=O] | 5-(5,5,8a-trimethyl- 2-methylene- decalin-1-yl)-3- methyl-pent-2-enal | | labda-8(20), 13-dien-15-al | |
| 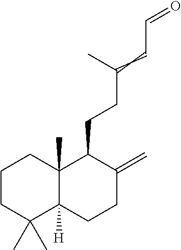 [SMILES: C=C1CC[C@H]2C(C)(C)CCC[C@]2(C)[C@H]1CCC(C)=CC=O] | 5-[(1S,4aS,8aS)- 5,5,8a-trimethyl-2- methylene-decalin- 1-yl]-3-methyl- pent-2-enal | | (5S,9S,10S)- labda-8(20), 13- dien-15-al | cis/trans- copalol |
| 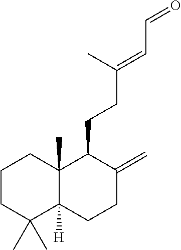 [SMILES: C=C1CC[C@H]2C(C)(C)CCC[C@]2(C)[C@H]1CC/C(C)=C/C=O] | (E)-5-[(1S,4aS,8aS)- 5,5,8a-trimethyl-2- methylene-decalin- 1-yl]-3-methyl- pent-2-enal | | (5S,9S,10S)- (13E)-labda- 8(20), 13-dien- 15-al | trans-copalol |
| 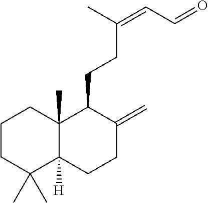 [SMILES: C=C1CC[C@H]2C(C)(C)CCC[C@]2(C)[C@H]1CC/C(C)=C\C=O] | (Z)-5-[(1S,4aS,8aS)- 5,5,8a-trimethyl-2- methylene-decalin- 1-yl]-3-methyl- pent-2-enal | | (5S,9S,10S)- (13Z)-labda- 8(20), 13-dien- 15-al | cis-copalal |
| 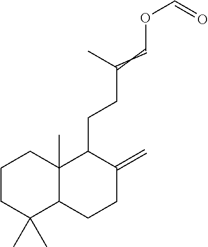 [SMILES: C=C1CCC2C(C)(C)CCCC2(C)C1CCC(C)=COC=O] | [4-(5,5,8a- trimethyl-2- methylene-decalin- 1-yl)-2-methyl-but- 1-enyl] formate | | (5S,9S,10S)-15- norlabda- 8(20),13-dien-14- yl formate | |
| 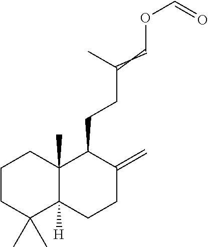 [SMILES: C=C1CC[C@H]2C(C)(C)CCC[C@]2(C)[C@H]1CCC(C)=COC=O] | [4-[(1S,4aS,8aS)- 5,5,8a-trimethyl-2- methylene-decalin- 1-yl]-2-methyl-but- 1-enyl]formate | 1a, 1b | (5S,9S,10S)-15- norlabda- 8(20), 13-dien-14- yl formate | |
| 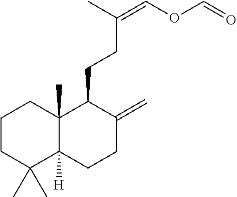 [SMILES: C=C1CC[C@H]2C(C)(C)CCC[C@]2(C)[C@H]1CC/C(C)=C\OC=O] | [(Z)- 4-[(1S,4aS,8aS)- 5,5,8a-trimethyl-2- methylene-decalin- 1-yl]-2-methyl-but- 1-enyl] formate | 1a | (5S,9S,10S)- (13Z)-15- norlabda- 8(20), 13-dien-14- yl formate | |
| 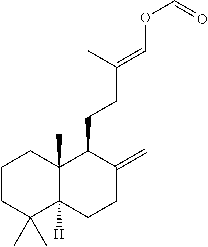 [SMILES: C=C1CC[C@H]2C(C)(C)CCC[C@]2(C)[C@H]1CC/C(C)=C/OC=O] | [(E)- 4-[(1S,4aS,8aS)- 5,5,8a-trimethyl-2- methylene-decalin- 1-yl]-2-methyl-but- 1-enyl] formate | 1b | (5S,9S,10S)- (13E)-15- norlabda- 8(20), 13-dien-14- yl formate | |
| 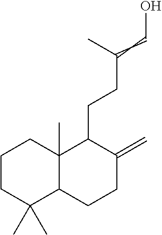 [SMILES: C=C1CCC2C(C)(C)CCCC2(C)C1CCC(C)=CO] | 4-(5,5,8a-trimethyl- 2-methylene- decalin-1-yl)-2- methyl-but-1-en-1- ol formate (not stable) | | 15-norlabda-8(20), 13-dien-14-ol | |
| 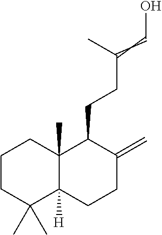 [SMILES: C=C1CC[C@H]2C(C)(C)CCC[C@]2(C)[C@H]1CCC(C)=CO] | 4-[(1S,4aS,8aS)- 5,5,8a-trimethyl-2- methylene-decalin- 1-yl]-2-methyl-but- 1-en-1-ol (not stable) | 2a, 2b | (5S,9S,10S)-15- norlabda-8(20), 13-dien-14-ol | |
| 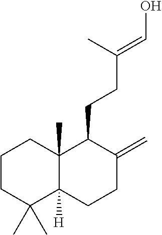 [SMILES: C=C1CC[C@H]2C(C)(C)CCC[C@]2(C)[C@H]1CC/C(C)=C/O] | (E)- 4-[(1S,4aS,8aS)- 5,5,8a-trimethyl-2- methylene-decalin- 1-yl]-2-methyl-but- 1-en-1-ol (not stable) | 2a | (5S,9S,10S)- (13E)-15- norlabda-8(20), 13-dien-14-ol | |
| 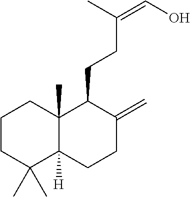 [SMILES: C=C1CC[C@H]2C(C)(C)CCC[C@]2(C)[C@H]1CC/C(C)=C\O] | (Z)- 4-[(1S,4aS,8aS)- 45,5,8a-trimethyl- 2-methylene- decalin-1-yl]-2- methyl-but-1-en-1-ol (not stable) | 2b | (5S,9S,10S)- (13Z)-15- norlabda-8(20), 13-dien-14-ol | |
| 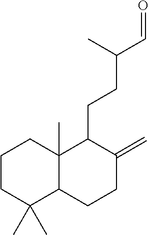 [SMILES: C=C1CCC2C(C)(C)CCCC2(C)C1CCC(C)C=O] | 4-(5,5,8a-trimethyl- 2-methylene- decalin-1-yl)-2- methyl-butanal | | 15-norlabd-8(20)- en-14-al | |
| 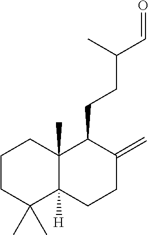 [SMILES: C=C1CC[C@H]2C(C)(C)CCC[C@]2(C)[C@H]1CCC(C)C=O] | 4-[(1S,4aS,8aS)- 5,5,8a-trimethyl-2- methylene-decalin- 1-yl]-2-methyl- butanal | 3a, 3b | (5S,9S,10S)- norlabd-8(20)-en- 14-al | |
| 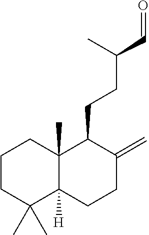 [SMILES: C=C1CC[C@H]2C(C)(C)CCC[C@]2(C)[C@H]1CC[C@@H](C)C=O] | (2R)-4- [(1S,4aS,8aS)- 5,5,8a-trimethyl- 2-methylene- decalin-1-yl]-2- methyl-butanal | 3a | (5S,9S,10S,13R)- 15-norlabd- 8(20)-en-14-al | |
| 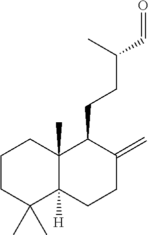 [SMILES: C=C1CC[C@H]2C(C)(C)CCC[C@]2(C)[C@H]1CC[C@H](C)C=O] | (2S)-4- [(1S,4aS,8aS)- 5,5,8a-trimethyl-2- methylene- decalin-1-yl]-2- methyl-butanal | 3b | (5S,9S,10S,13S)- 15-norlabd- 8(20)-en-14-al | |
| 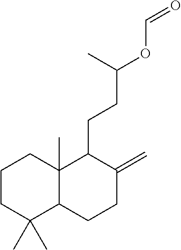 [SMILES: C=C1CCC2C(C)(C)CCCC2(C)C1CCC(C)OC=O] | [3-(5,5,8a-trimethyl-2- methylene-decalin- 1-yl]-1-methyl- propyl] formate | | 14,15-dinorlabd- 8(20)-en-13-yl formate | |
| 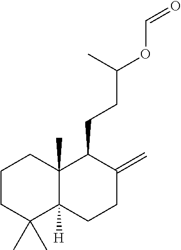 [SMILES: C=C1CC[C@H]2C(C)(C)CCC[C@]2(C)[C@H]1CCC(C)OC=O] | [3-[(1S,4aS,8aS)- 5,5,8a-trimethyl-2- methylene-decalin- 1-yl]-1-methyl- propyl] formate | 4a, 4b | (5S,9S,10S)- 14,15-dinorlabd- 8(20)-en-13-yl formate | |
| 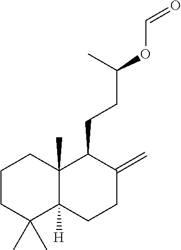 [SMILES: C=C1CC[C@H]2C(C)(C)CCC[C@]2(C)[C@H]1CC[C@@H](C)OC=O] | [(1R)- 3-[(1S,4aS,8aS)- 5,5,8a-trimethyl-2- methylene-decalin- 1-yl]-1-methyl- propyl]formate | 4a | (5S,9S,10S,13R)- 14,15-dinorlabd- 8(20)-en-13-yl formate | |
| 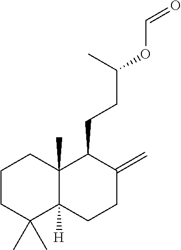 [SMILES: C=C1CC[C@H]2C(C)(C)CCC[C@]2(C)[C@H]1CC[C@H](C)OC=O] | [(1S)- 3-[(1S,4aS,8aS)- 5,5,8a-trimethyl-2- methylene-decalin- 1-yl]-1-methyl- propyl] formate | 4b | (5S,9S,10S,13R)- 14,15-dinorlabd- 8(20)-en-13-yl formate | |
| 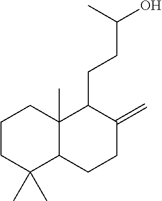 [SMILES: C=C1CCC2C(C)(C)CCCC2(C)C1CCC(C)O] | 4-(5,5,8a-trimethyl- 2-methylene- decalin-1-yl)butan- 2-ol | | 14,15-dinorlabd- 8(20)-en-13-ol | |
| 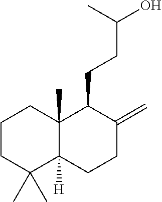 [SMILES: C=C1CC[C@H]2C(C)(C)CCC[C@]2(C)[C@H]1CCC(C)O] | 4-[(1S,4aS,8aS)- 5,5,8a-trimethyl-2- methylene-decalin- 1-yl] butan-2-ol | 5a, 5b | (5S,9S,10S)- 14,15-dinorlabd- 8(20)-en-13-ol | |
| 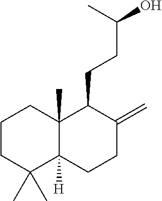 [SMILES: C=C1CC[C@H]2C(C)(C)CCC[C@]2(C)[C@H]1CC[C@@H](C)O] | (2R)- 4-[(1S,4aS,8aS)- 5,5,8a-trimethyl-2- methylene-decalin- 1-yl]butan-2-ol | 5a | (5S,9S,10S,13R)- 14,15-dinorlabd- 8(20)-en-13-ol | |
| 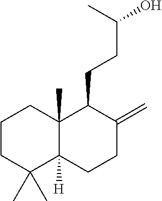 [SMILES: C=C1CC[C@H]2C(C)(C)CCC[C@]2(C)[C@H]1CC[C@H](C)O] | (2S)- 4-[(1S,4aS,8aS)- 5,5,8a-trimethyl-2- methylene-decalin- 1-yl]butan-2-ol | 5b | (5S,9S,10S,13S)- 14,15-dinorlabd- 8(20)-en-13-ol | |
| 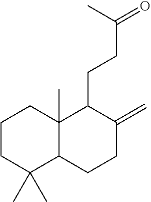 [SMILES: C=C1CCC2C(C)(C)CCCC2(C)C1CCC(C)=O] | 4-5(,5,8a-trimethyl-2- 2-methylene- decalin-1-yl)butan- 2-one | | 14,15-dinorlabd- 8(20)-en-13-one | |
| 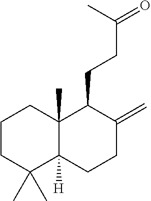 [SMILES: C=C1CC[C@H]2C(C)(C)CCC[C@]2(C)[C@H]1CCC(C)=O] | 4-[(1S,4aS,8aS)- 5,5,8a-trimethyl-2- methylene-decalin- l-yl]butan-2-one | | (5S,9S,10S)- 14,15-dinorlabd- 8(20)-en-13-one | (+)-manooloxy |
| 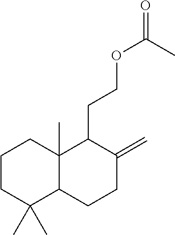 [SMILES: C=C1CCC2C(C)(C)CCCC2(C)C1CCOC(C)=O] | 2-(5,5,8a-trimethyl- 2-methylene- decalin-1-yl)ethyl acetate | | 13,14,15,16- tetranor-labda- 8(20)-en-12-yl acetate | 13,14,15,16- tetranorlabdenyl acetate |
| 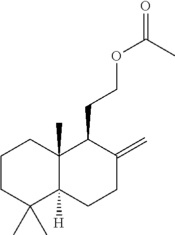 [SMILES: C=C1CC[C@H]2C(C)(C)CCC[C@]2(C)[C@H]1CCOC(C)=O] | 2-[(1S,4aS,8aS)- 5,5,8a-trimethyl-2- methylene-decalin- 1-yl]ethyl acetate | | (5S,9R,10S)- 13,14,15,16- tetranor-labde- 8(20)-en-12-yl acetate | (+)-γ-ambryl acetate |
| 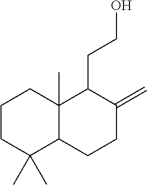 [SMILES: C=C1CCC2C(C)(C)CCCC2(C)C1CCO] | 2-(5,5,8a-trimethyl- 2-methylene-decalin-1- yl)ethanol | | 13,14,15,16- tetranorlabda- 8(20)-en-12-ol | 13,14,15,16- tetranorlabden- ol |
| 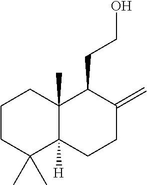 [SMILES: C=C1CC[C@H]2C(C)(C)CCC[C@]2(C)[C@H]1CCO] | 2-[(1S,4aS,8aS)- 5,5,8a-trimethyl-2- methylene-decalin- 1-yl]ethanol | | (5S,9S,10S)- 13,14,15,16- tetranorlabda- 8(20)-en-12-ol | (+)-γ-ambrol |
| 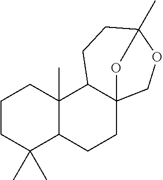 [SMILES: CC12CCC3C(CCC4C(C)(C)CCCC43C)(CO1)O2] | 3,8,8,11a- tetramethyldodecahy- dro-3,5a-epoxynaphtho [2,1-c]oxepin | | 8,13:13,20- diepoxy-15,16- dinorlabdane | diepoxy- dinorlabdane |
| 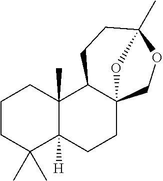 [SMILES: CC1(C)CCC[C@@]2(C)[C@H]1CC[C@]13CO[C@](C)(CC[C@@H]12)O3] | (3S,5aR,7aS,11aS, 11bR)-3,8,8,11a- tetramethyldodecahydro- 3,5a-epoxynaphtho [2,1- c]oxepin | | (5S,8R,9R,10S, 13S)-8, 13:13,20- diepoxy-15,16- dinorlabdane | Z-11 |
| 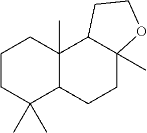 [SMILES: CC1(C)CCCC2(C)C1CCC1(C)OCCC12] | 3a,6,6,9a- tetramethyl- 2,4,5,5a,7,8,9,9b- octahydro-1H- benzo[e]benzofuran | 8,12-epoxy- 13,14,15,16- tetranorlabdane | | epoxy- tetranorlabdane |
| 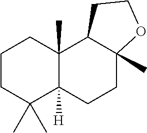 [SMILES: CC1(C)CCC[C@]2(C)[C@H]3CCO[C@]3(C)CC[C@@H]12] | (3aR,5aS,9aS,9bR)- 3a,6,6,9a- tetramethyl- 2,4,5,5a,7,8,9,9b- octahydro-1H- benzo[e]benzofuran | | (5S,8R,9R,10S)- 8,12-epoxy- 13,14,15,16- tetranorlabdane | Ambrox |
| 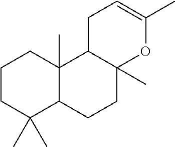 [SMILES: CC1=CCC2C(C)(CCC3C(C)(C)CCCC32C)O1] | 3,4a,7,7,10a- pentamethyl- 1,5,6,6a,8,9,10,10b- octahydrobenzo[f] chromene | | 8,13-epoxy- 13,14,15,16- dinorlabd-12-ene | |
| 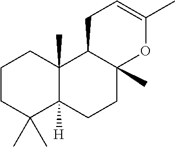 [SMILES: CC1=CC[C@@H]2[C@@]3(C)CCCC(C)(C)[C@@H]3CC[C@@]2(C)O1] | (4aR,6aS,10aS,10b R)-3,4a,7,7,10a- pentamethyl- l,5,6,6a,8,9,10,10b- octahydrobenzo[f] chromene | | (5S,8R,9R,10S)- 8,13-epoxy- 13,14,15,16- dinorlabd-12-ene | sclareol oxide |

### DETAILED DESCRIPTION

### a. Particular Embodiments of the Invention

i) The present invention relates to the following particular embodiments of biocatalytic methods involving the use of polypetides with BMVO activity:

1. A biocatalytic method for preparing an ester compound, comprising:
(1) contacting a carbonyl precursor compound of general formula I

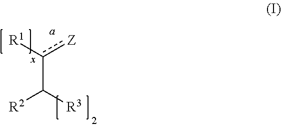

wherein
   “a” denotes a single or double bond,
      “x” is integer 1 if “a” denotes a double bond, or “x” is integer 2 if “a” denotes a
      single bond,
      R¹ represent independently of each other H or lower alkyl, like C₁-C₄-alkyl, in
      particular H or methyl,
      R² represents H, a linear or branched, saturated or unsaturated, optionally substituted hydrocarbyl residue, in particular having 2 to 20, more particularly 5 to 15 carbon atoms, or a group Cyc-A-,
         wherein
         Cyc represents an optionally substituted, saturated or unsaturated, mono- or polycyclic hydrocarbyl residue, and
         A represents a chemical bond or an optionally substituted, straight chain or branched alkylene bridge, in particular methylene,
      R³ represent independently of each other H or a C₁-C₃₀, C₁-C₂₀ or in particular C₁-C₁₅ hydrocarbyl group, or a lower alkyl group, like C₁-C₄-alkyl, in particular H or methyl, and more particularly are each H, and
      when “a” denotes a single bond, then Z represents a hydrocarbyl residue containing a carbonyl group, in particular aldehyde or keto group, or,
      when “a” denotes a double bond, then Z forms, together with the carbon atom which it is attached to, either a carbonyl group (C═O, in particular aldehyde or keto group, or an alkylidene residue, in particular a C₁-C₆-alkylidene, residue carrying a terminal carbonyl group, in particular aldehyde or keto group, and when “a” denotes a double bond, and Z forms, together with the carbon atom which it is attached to, either a carbonyl group (C═O), then R² and R¹, together with the carbon atoms which they are attached to, may also form a cyclic, in particular monocyclic, saturated or unsaturated, optionally substituted carbocyclic ring group, in particular 5-7-membered ring:
      wherein said carbonyl compound of general formula I is provided in stereoisomerically pure form, or as a mixture of stereoisomers:
   with a natural or recombinant polypeptide having Baeyer-Villiger monooxygenase (BVMO) (EC 1.13.14.-) activity so as to form the respective carbonyl ester product, in particular by introducing a oxygen atom between the carbonyl group and the alpha-carbon atom of the precursor,
(2) and optionally isolating the carbonyl ester formed in step (1), wherein said carbonyl ester compound is obtained in stereoisomerically pure form, or as a mixture of stereoisomers.

2. The biocatalytic method of embodiment 1, wherein in the carbonyl compound of general formula I
“a” represents a chemical double bond and Z represents ═O (cf. Manooloxy) or ═C(R⁴)—C(R⁵)—O (cf. Copalal); or
“a” represents a chemical single bond and Z represents-C(R⁵)—O (cf. Norlabdane compound 3a,3b);
   wherein
   R⁴ and R⁵ independently of each other represent H or lower alkyl, like C₁-C₄-alkyl, in particular H or methyl.

3. The biocatalytic method of anyone of the preceding embodiments, wherein the carbonyl compound of general formula I possesses a labdane-type structure, in particular a labdane, norlabdane or di-norlabdane structure.

4. The biocatalytic method of anyone of the preceding embodiments wherein the carbonyl ester formed is of the formula II

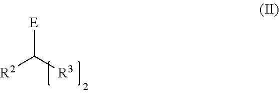

wherein
R² and R³ are as defined above, and
E represents a hydrocarbyl residue containing said carbonyl ester group, or wherein E and R² together with the carbon atom which they are attached to form a cyclic ester group.

5. The biocatalytic method of embodiment 4, wherein the carbonyl ester group E is selected from
—O—C(O)—R¹,
—C(R¹)₂—O—C(O) R⁵,
—C(R¹)═C(R⁴)—O—C(R⁵)═O; and
a cyclic ester group formed by E and R² together with the carbon atom which they are attached to, wherein the cyclic ester ring represents a 5- to 7-membered, in particular 6-membered ring, as for example in esters of the formulae Ila and IIb

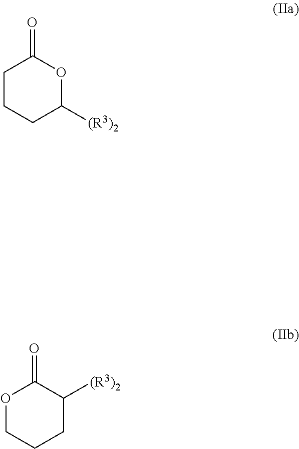

wherein R¹, R³, R⁴ and R⁵ are as defined above.

6. The biocatalytic method of anyone of the preceding embodiments, wherein R² represents a group Cyc-A-, wherein A represents a straight chain or branched C₁-C₄-alkylene bridge, in particular methylene, and Cyc represents a mono- or polycyclic, in particular bicyclic, saturated or unsaturated hydrocarbyl residue, in particular a bicyclic anellated hydrocarbyl residue comprising 5-7, in particular 6, ring atoms per cycle; wherein Cyc is optionally substituted with 1-10, in particular 1-5 substituents, wherein said substituents in particular may be independently selected from C₁-C₄-alkyl, C₁-C₄-alkylidene, C₃-C₆-alkenylidene, C₂-C₄-alkenyl, oxo (═O), hydroxy, or amino; and in particular C₁-C₄-alkyl, like methyl, and C₁-C₄-alkylidene, like methylidene.

7. The method of anyone of the preceding embodiments, wherein the Cyc residue of R² forms an optionally substituted decalinyl residue, like in particular bicyclic residue obtainable through terpene cyclization.

8. The method of embodiment 7, wherein Cyc-A represents a bicyclic residue having 15 carbon atoms of formula IIIa, IIIb or IIIc

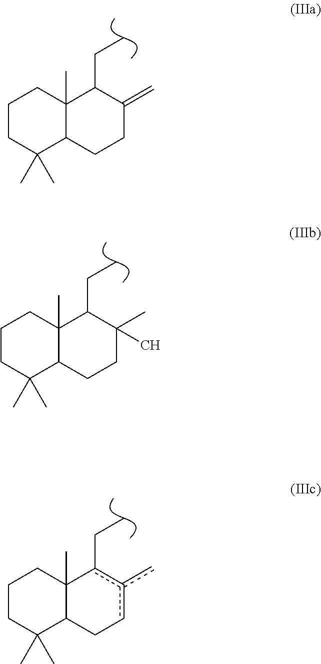

9. The method of anyone of the preceding embodiments, wherein the polypeptide having BVMO activity is selected from:
(1) the group of polypeptides containing a flavin-containing monooxygenase (FMO) protein family domain having the Pfam ID number PF00743 within their amino acid sequence; or a domain retaining at least 90%, 95%, 96%, 97%, 98%, or 99% sequence identity to PF00743;
   In particular, a polypeptide of the invention having BVMO activity is identified as member of the FMO protein family comprising said domain PF00743 if it matches with said domain with an e-value of less than 1×10⁻⁵ or less than 1×10⁻¹⁰, or less than or equal to 1×10⁻¹⁵, or less than or equal to 1×10⁻¹⁸., in particular in a range of 1×10⁻¹⁰ to 1×10⁻¹⁸ and more particular in a range of 1×10⁻¹⁴ to 1×10⁻¹⁷. As the query sequence the sequence of a polypeptide having BVMO activity is applied.
   For example, the following website may be applied for the search and calculating such e-value: pfam.\fam.org/, www.ebi.ac.uk/Tools/hmmer/search/hmmscan or www.ebi.ac.uk/Tools/pfa/pfamscan/.
and/or
(2) selected from the group of polypeptides that comprise at least 1, 2, 3, 4, 5, 6, 7 or all of the sequence motif/domain selected from

GAGxSGL set forth in SEQ ID NO: 197

EKNxxxxGTWxENRYPGCACDVPxHxYXXSFE set forth in SEQ ID NO: 198 or any partial motif thereof comprising up to 15, up to 10 or up to 5 consecutive amino acid residues, as for example corresponding to residues in positions 1-10, 11-20 or 21-32 of SEQ ID NO: 198;

LxNAxGILNxWxxPxIPG set forth in SEQ ID NO: 199

or any partial motif thereof comprising up to 15, up to 10 or up to 5 consecutive amino acid residues, as for example corresponding to residues in positions 1-10 or 11-18 of SEQ ID NO:199;

LxxKxVxxIGxGSSGIQIxPxI set forth in SEQ ID NO: 200

or any partial motif thereof comprising up to 15, up to 10 or up to 5 consecutive amino acid residues, as for example corresponding to residues in positions 1-10 or 11-18 of SEQ ID NO:200;

GCRRxTPGxxYLExL set forth in SEQ ID NO: 201

or any partial motif thereof comprising up to 15, up to 10 or up to 5 consecutive amino acid residues, as for example corresponding to residues in positions 1-10, 11-15 of SEQ ID NO:201:

CATGFDxxxxPRFxxxG set forth in SEQ ID NO: 202

or any partial motif thereof comprising up to 15, up to 10 or up to 5 consecutive amino acid residues, as for example corresponding to residues in positions 1-10 or 11-17 of SEQ ID NO:202

PNxFxxxGPNxPxxNGxV set forth in SEQ ID NO: 203

or any partial motif thereof comprising up to 15, up to 10 or up to 5 consecutive amino acid residues, as for example corresponding to residues in positions 1-10 or 11-18 of SEQ ID NO:203:

AxWPGSxLHYxEAxxxPRxED set forth in SEQ ID NO: 204

or any partial motif thereof comprising up to 15, up to 10 or up to 5 consecutive amino acid residues, as for example corresponding to residues in positions 1-10 or 11-21 of SEQ ID NO:204;
wherein
in the above motifs residues x represent independently of each other any natural amino acid residue, and wherein optionally in each of the above motifs 1 to 5, like 1, 2, 3, 4 or 5 of the conserved amino acid residues (i.e. different from the x residues) may be modified, for example by amino acid substitution, in particular by conservative substitutions, provided that the enzymes retains, at least to analytically detectable extent, BVMO enzyme activity.
   and/or
(3) the group of polypeptides consisting of
   (a) polypeptides comprising the amino acid sequence of SCH23-BVMO1 set forth in SEQ ID NO:2;
   (b) polypeptides comprising the amino acid sequence of SCH24-BVMO1 set forth in SEQ ID NO:6;
   (c) polypeptides comprising the amino acid sequence of SCH25-BVMO1 set forth in SEQ ID NO:10;
   (d) polypeptides comprising the amino acid sequence of SCH46-BVMO1 set forth in SEQ ID NO:13;
   (e) polypeptides comprising the amino acid sequence of AspWeBVMO set forth in SEQ ID NO: 16 (preferential substrate Manooloxa and its isomers)
   (f) polypeptides comprising an amino acid sequence that has at least 70%, 75%, 80%, 85%, 90%, 95%, 96%, 97%, 98%, or 99% identity to any one of the amino acid sequences of a) to e).

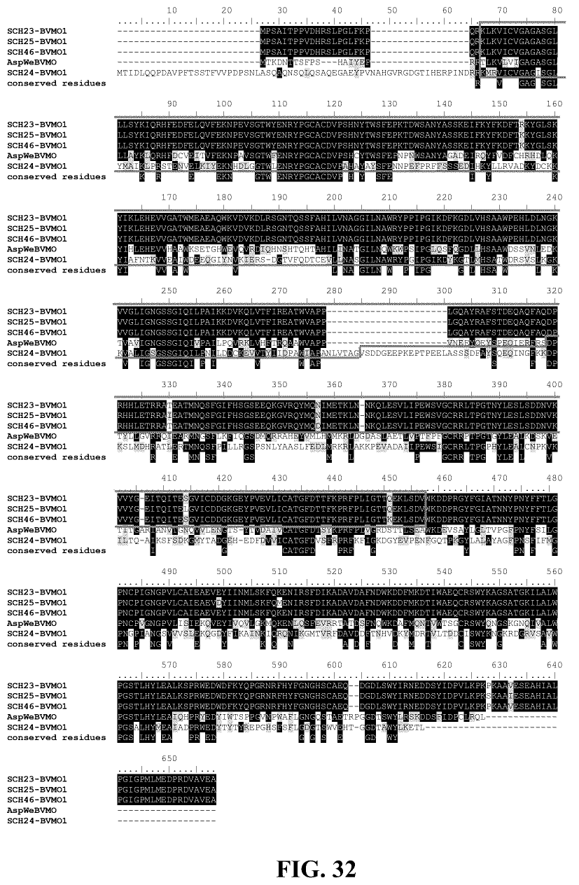

In the above referenced five particular BVMO polypeptides the protein family domain having the Pfam ID number PF00743 may be located at amino acid residue positions given in the following table (see also alignment depicted in FIG. 32 and the framed sequence sections therein)

| Protein | | | | Accession | |
|---|---|---|---|---|---|
| sequence | From | To | E-Value | Id | Protein domain |
| SCH23-BVMO1 | 23 | 388 | 2.9e−16 | Pf00743 | Flavin-binding |
| | | | | | monooxygenase-like |
| SCH24-BVMO2 | 67 | 283 | 6.8e−15 | Pf00743 | Flavin-binding |
| | | | | | monooxygenase-like |
| SCH25-BVMO1 | 23 | 246 | 1.2e−15 | Pf00743 | Flavin-binding |
| | | | | | monooxygenase-like |
| SCH46-BVMO1 | 23 | 388 | 1.8e−16 | Pf00743 | Flavin-binding |
| | | | | | monooxygenase-like |
| AspWe BVMO | 20 | 249 | 1.7e−16 | Pf00743 | Flavin-binding |
| | | | | | monooxygenase-like |

The numbering of amino acid residues refers to the residue number in the respective SEQ ID NO of the respective protein sequence in the attached sequence listing

Another particular embodiment refers to polypeptide variants of the novel polypeptides of the invention having a BVMO activity as identified above by anyone of the particular amino acid sequences of SEQ ID NO: 2, 6, 10, and 13, and wherein the polypeptide variants are selected from an amino acid sequence having at least 70%, 75%, 80%, 85%, 90%, 95%, 96%, 97%, 98%, or 99% sequence identity to anyone of SEQ ID NO: 2, 6, 10, 13 and 16, and contain at least one substitution modification relative to anyone of the non-modified SEQ ID NO: 2, 6, 10, 13 and 16.

10. The method of anyone of the preceding embodiments performed in vitro or in vitro.

11. The method of embodiment 10 performed in vivo, that comprises, prior to step (1), the recombinant expression, in particular in a non-human host cell, of one or more polypeptides having the enzyme activity required for performing the BVMO catalyzed enzymatic step.

12. The method of embodiment 11, wherein the non-human host cell is transformed with a nucleic acid which is encoding at least one polypeptide having BVMO activity

13. The method of embodiment 11 or 12, wherein said non-human host cell is a eukaryotic or a prokaryotic cell, in particular a plant cell, a bacterial or a fungal cell, in particular a yeast cell.

14. The method of any one of embodiments 11 to 13, wherein the non-human host cell is a unicellular organism, a cultured cell derived from a multi-cellular organism, a cell present in a cultured tissue derived from a multicellular organism, or a cell present in a living multicellular organism.

15. The method of one of the embodiments 10 to 13, wherein the non-human host cell is a bacterium of the genus Escherichia, in particular E. coli and said yeast is of the genus Saccharomyces, or Pichia, in particular S. cerevisiae, or a plant cell.

16. The method of one of the preceding embodiments, wherein the carbonyl compound of general formula I is a labdane-type compound, selected from
a) a labdane aldehyde, in particular copalal (or any stereoisomerically different form thereof, for example comprising cis- or trans-form or a mixture of cis- and trans-forms) which is converted by said BVMO to the respective norlabdane formate, in particular particular (5S,9S,10S)-15-norlabda-8 (20),13-dien-14-yl-formate or any stereoisomerically different form thereof;
b) a dinorlabdane ketone, in particular manooloxy or any stereoisomerically different form thereof, which is converted by said BVMO to gamma-ambryl acetate or any stereoisomerically different form thereof; or
c) a norlabdane aldehyde, in particular the C₁-degraded analog of copalal or any stereoisomerically different form thereof, in particular of the formula

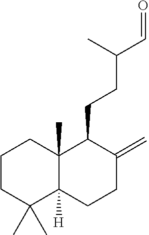 [SMILES: C=C1CC[C@H]2C(C)(C)CCC[C@]2(C)[C@H]1CCC(C)C=O]

or any stereoisomerically different form thereof
which is converted by said BVMO to the respective dinorlabdane formate ester in particular of the formula

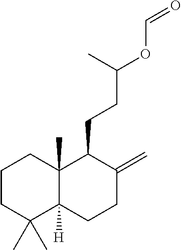 [SMILES: C=C1CC[C@H]2C(C)(C)CCC[C@]2(C)[C@H]1CCC(C)OC=O]

or any stereoisomerically different form thereof,
   and wherein optionally the obtained product is isolated in stereoisomerically essentially pure form or as a mixture of stereoisomers.

Further particular inventive examples of BVMO-catalyzed conversions of carbonyl compounds to the respective ester are summarized in the following schematic overview:

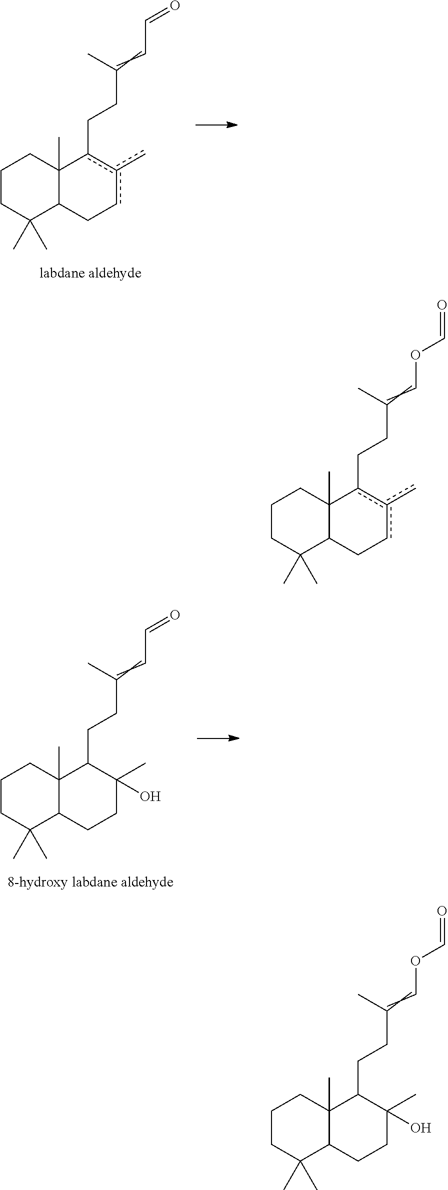

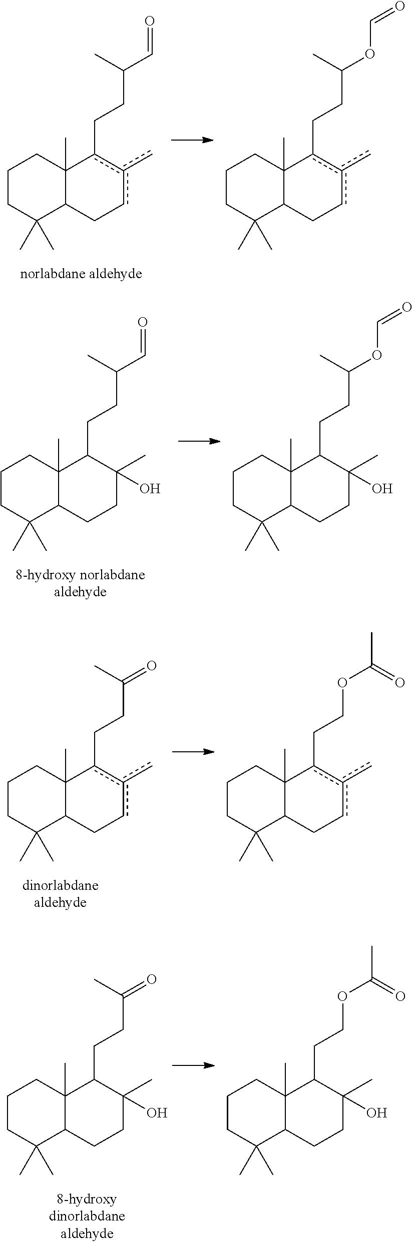

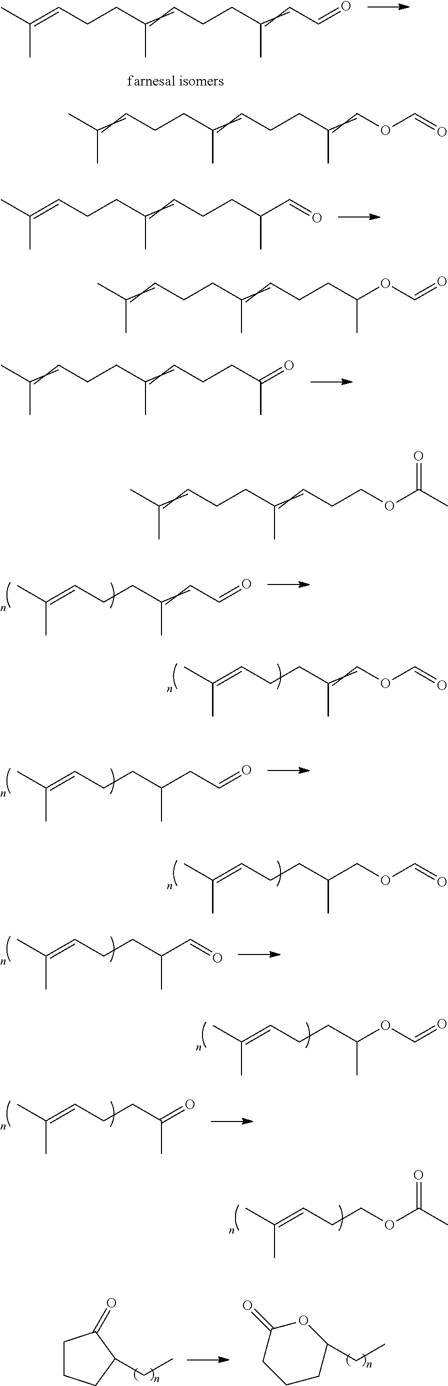

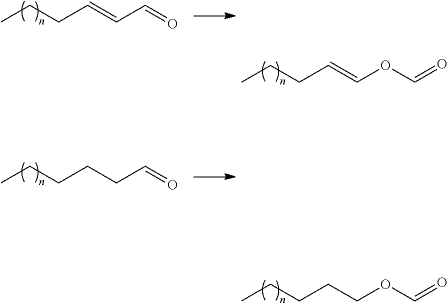

wherein parameter “n” is an integer from 1 to 20, 1 to 15, 1 to 10 or 1, 2, 3, 4 or 5.

17. The method of embodiment 16a, which comprises prior to step (1) the biocatalytic oxidation of a labdane alcohol to a labdane aldehyde, in particular of copalol to copalal,
which labdane alcohol is optionally formed by the biocatalytic conversion of at least one terpenly diphosphate precursor, selected from IPP, DMAPP, FPP and GGPP, in particular in a single step or a combination of at least two steps, known in the prior art.

Said labdane alcohol may for example be biocatalytically produced:
a) from geranylgeranyl diphosphate (GGPP) in one step in a cyclisation reaction/dephosphorylation reaction
b) from GGPP in two steps by a cyclisation forming labdane diphosphate, as for example copalyl diphosphate (CPP) which is then dephosphorylated to the labdane alcohol;
c) from IPP and DMAPP which is directly converted through the action of a bifunctional GGPP synthase/CPP synthase to the labdane diphosphate, as for example CPP which is then dephosphorylated;
GGPP as used in these steps may also be provided by different biocatalytic steps:
d) GGPP synthases are available which produce GGPP directly from IPP and DMAPP; or
e) GGPP may be provided from IPP and DMAPP via FPP through the action of a FPP synthase, and the subsequent conversion of FPP to GGPP through the action of a GGPP synthase.

18. The method of embodiment 17, wherein
said biocatalytic oxidation of a labdane alcohol, in particular of copalol to copalal, is catalyzed by an exogenous or endogenous polypeptide having alcohol dehydrogenase (ADH) (EC 1.1.1.-) activity; and/or
said biocatalytic formation of the labdane alcohol comprises at least one step selected from
i) a biocatalytic dephosphorylation of a labdane diphosphate to a labdane alcohol, in particular of copalyl diphosphate (CPP) to copalol, which is catalyzed by a polypeptide having terpenyl diphosphate (TPP) phosphatase activity, and/or
ii) a biocatalytic cyclisation of a terpenly diphosphate precursor, as for example of geranylgeranyl diphosphate (GGPP) to CPP, which is catalyzed by a polypeptide having CPP synthase activity, like SmCPS2 (SEQ ID NO:185); or as for example of IPP and DMAPP to CPP, which is catalyzed by a bifunctional polypeptide having prenyl-transferase and copalyl-diphosphate synthase activity, like PvCPS, and/or
iii) a biocatalytic formation of GGPP from FPP or a biocatalytic formation from IPP and DMAPP, each of which being catalyzed by a polypeptide having GGPP synthase activity.

19. The method of embodiment 18, wherein
said biocatalytic oxidation, in particular of copalol to copalal, is catalyzed by a polypeptide having alcohol dehydrogenase (ADH) activity selected from
a) polypeptides comprising the amino acid sequence of SCH23-ADH1_wt set forth in SEQ ID NO: 134
b) polypeptides comprising the amino acid sequence of SCH24-ADH1_wt set forth in SEQ ID NO: 140
c) polypeptides comprising the amino acid sequence of SCH94-3945_wt set forth in SEQ ID NO:161
d) polypeptides comprising the amino acid sequence of SCH80-0540_wt set forth in SEQ ID NO:164
e) polypeptides comprising the amino acid sequence of AzTolADH1_wt set forth in SEQ ID NO:167
f) polypeptides comprising the amino acid sequence of CdGeoA_wt set forth in SEQ ID NO: 179
g) polypeptides comprising an amino acid sequence that has at least 70%, 75%, 80%, 85%, 90%, 95%, 96%, 97%, 98%, or 99% identity to any one of the amino acid sequences of a) to f) and having ADH activity.
and/or
said biocatalytic dephosphorylation, in particular of copalyl diphosphate (CPP) to copalol, is catalyzed by a polypeptide having terpenyl diphosphate (TPP) phosphatase activity selected from
a) polypeptides comprising an amino acid sequence of AspWE TPP as set forth in SEQ ID NO:170 or a polypeptide comprising an amino acid sequence that has at least 70%, 75%, 80%, 85%, 90%, 95%, 96%, 97%, 98%, or 99% identity thereto;
b) polypeptides comprising an amino acid sequence of TalCeTPP as set forth in SEQ ID NO: 176 or a polypeptide comprising an amino acid sequence that has at least 70%, 75%, 80%, 85%, 90%, 95%, 96%, 97%, 98%, or 99% identity thereto; and
c) polypeptides comprising an amino acid sequence of TalVeTPP as set forth in SEQ ID NO: 194 or a polypeptide comprising an amino acid sequence that has at least 70%, 75%, 80%, 85%, 90%, 95%, 96%, 97%, 98%, or 99% identity thereto;

Further suitable phosphatases are also disclosed in earlier the applicant's EP Application number 18182783.3, incorporated by reference.
and/or
said biocatalytic cyclisation, in particular of geranylgeranyl diphosphate (GGPP) to CPP, is catalyzed by a polypeptide selected from
   polypeptides having copalyl-diphosphate synthase activity comprising the amino acid sequence of SmCPS2 as set forth in SEQ ID NO:185 or a polypeptide comprising an amino acid sequence that has at least 70%, 75%, 80%, 85%, 90%, 95%, 96%, 97%, 98%, or 99% 70% identity thereto;
said biocatalytic cyclisation, in particular of IPP and DNMAPP to CPP, is catalyzed by
a polypeptide selected from
   polypeptides having prenyl-transferase and copalyl-diphosphate synthase activities comprising the amino acid sequence of PvCPS as set forth in SEQ ID NO:173 or a polypeptide comprising an amino acid sequence that has at least 70%, 75%, 80%, 85%, 90%, 95%, 96%, 97%, 98%, or 99% identity thereto;
   and/or
said biocatalytic formation of GGPP is catalyzed by a polypeptide having GGPP
synthase activity and is selected from
a) polypeptides comprising the amino acid sequence of carG as set forth in SEQ ID NO: 182 or a polypeptide comprising an amino acid sequence that has at least 70%, 75%, 80%, 85%, 90%, 95%, 96%, 97%, 98%, or 99% identity thereto;
b) polypeptides comprising the amino acid sequence of CrtE as set forth in SEQ ID NO: 191 or a polypeptide comprising an amino acid sequence that has at least 70%, 75%, 80%, 85%, 90%, 95%, 96%, 97%, 98%, or 99% identity thereto;
c) polypeptides comprising the amino acid sequence of PvCPS as set forth in SEQ ID NO: 173 or a polypeptide comprising an amino acid sequence that has at least 70%, 75%, 80%, 85%, 90%, 95%, 96%, 97%, 98%, or 99% identity thereto.

20. The method of anyone of the preceding embodiments further comprising as step (3) the processing of the carbonyl ester formed in step (1) or isolated in step (2) to obtain a derivative thereof using chemical or biocatalytic synthesis or a combination of both, wherein said derivative may in particular be selected from a hydrocarbon, alcohol, diol, triol, acetal, ketal, aldehyde, acid, ether, amide, ketone, lactone, epoxide, acetate, glycoside and/or an ester, and optionally isolating the derivative of step (3).

21. The method of embodiment 20, wherein step (3) comprises the hydrolysis of the carbonyl ester compound with an esterase activity EC 3.1.1 (Carboxylic Ester Hydrolases) to the corresponding de-esterified product (which may be an alcohol or an isomerization product thereof), and optionally isolating the derivative of step (3).

22. The method of embodiment 21, wherein the de-esterified product of step (3) is subjected in a further step (4) to an enzymatic redox reaction, wherein in particular the redox reaction comprises the oxidation of an alcohol group as formed in step (3) to the corresponding keto-group through the enzymatic action of an an exogenous or endogenous alcohol dehydrogenase (ADH) (EC 1.1.1.-).

23. The method of embodiment 21, wherein the esterase is selected from the group consisting of
a) polypeptides comprising the amino acid sequence of SCH23-Esterase set forth in SEQ ID NO:20;
b) polypeptides comprising the amino acid sequence of SCH24-Esterase set forth in SEQ ID NO:24;
c) polypeptides comprising the amino acid sequence of SCH25-Esterase set forth in SEQ ID NO:28;
d) polypeptides comprising the amino acid sequence of SCH46-Esterase set forth in SEQ ID NO:31; or
e) polypeptides comprising an amino acid sequence that has at least 70%, 75%, 80%, 85%, 90%, 95%, 96%, 97%, 98%, or 99% identity to any one of the amino acid sequences of a) to d) and having esterase activity.

24. The method of embodiment 23, wherein
a) a norlabdane ester, in particular a norlabdane-formate is de-esterified by said esterase to a norlabdane carbonyl compound, in particular a carbonyl compound if the formula

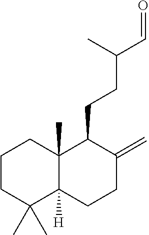 [SMILES: C=C1CC[C@H]2C(C)(C)CCC[C@]2(C)[C@H]1CCC(C)C=O]

or the respective enol thereof which is the converted via isomerisation to said carbonyl compound;
or
b) a tetranorlabdane ester, in particular gamma-ambryl acetate is de-esterified by said esterase to a tetranorlabdane, in particular gamma ambrol; or
c) a dinorlabdane formate ester, in particular the formate ester of the formula

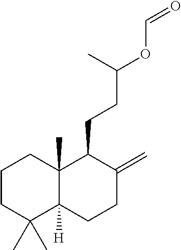 [SMILES: C=C1CC[C@H]2C(C)(C)CCC[C@]2(C)[C@H]1CCC(C)OC=O]

or any stereoisomerically different form thereof
   is de-esterified by said esterase to the corresponding dinorlabdane alcohol, in particular to the alcohol-compound of the formula

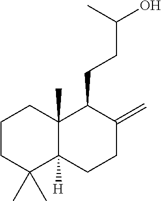 [SMILES: C=C1CC[C@H]2C(C)(C)CCC[C@]2(C)[C@H]1CCC(C)O]

or any stereoisomerically different form thereof
and wherein optionally the obtained product is isolated in stereoisomerically essentially pure form or as a mixture of stereoisomers.

25. The method of embodiment 22, wherein the ADH is selected from the group consisting of
a) polypeptides comprising the amino acid sequence of SCH23-ADH2_wt set forth in SEQ ID NO: 137
b) polypeptides comprising the amino acid sequence of SCH24-ADH2_wt set forth in SEQ ID NO: 143
c) polypeptides comprising the amino acid sequence of RrhSecADH_wt set forth in SEQ ID NO:146
d) polypeptides comprising the amino acid sequence of SCH80-06135_wt set forth in SEQ ID NO:155
e) polypeptides comprising an amino acid sequence that has at least 70%, 75%, 80%, 85%, 90%, 95%, 96%, 97%, 98%, or 99% identity to any one of the amino acid sequences of a) to d) and having ADH activity.

26. The method of embodiment 24, wherein the obtained dinorlabdane alcohol, in particular the alcohol of the formula

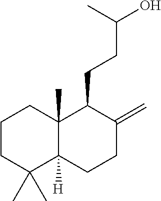 [SMILES: C=C1CC[C@H]2C(C)(C)CCC[C@]2(C)[C@H]1CCC(C)O]

or any stereoisomerically different form thereof
is oxidized by said ADH to the corresponding dinorlabdane carbonyl compound, in particular to manoooloxy,
and wherein optionally the obtained product is isolated in stereoisomerically essentially pure form or as a mixture of stereoisomers.

ii) The present invention relates to the following particular embodiments of biocatalytic methods involving the use of polypeptides with enal-cleaving activity:

27. A biocatalytic method of preparing a compound of the general formula IV

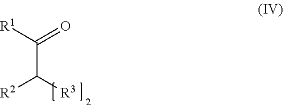

wherein
R¹ represents H or lower alkyl, in particular methyl,
R² represents H, a linear or branched, saturated or unsaturated, optionally substituted hydrocarbyl group, in particular alkyl or alkenyl group, in particular having up to 30, up to 20, up to 15 or up to 10 carbon atoms, or a residue Cyc-A-
   wherein
   Cyc represents an optionally substituted, saturated or unsaturated, in particular nonaromatic, mono- or polycyclic, in particular mono- or bicyclic, hydrocarbyl residue, in particular having 5 to 7 ring carbon atoms, and
   A represents a chemical bond or an optionally substituted, straight chain or branched alkylene bridge, in particular methylene,
   and
R³ represent independently of each other H or lower alkyl, like C₁-C₄-alkyl, in particular H or methyl, and more particularly are each H,
comprising the steps of
   (1) contacting the corresponding non-degraded precursor of the general formula V

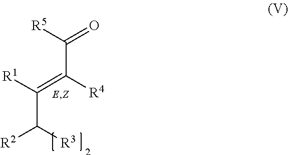

wherein
   R¹, R² and R³ are as defined above; and
   R⁴ represents H or lower alkyl, in particular H or methyl,
   R⁵ represents H or lower alkyl, in particular H,
   and wherein said compound may be present in stereoisomerically essentially pure form (as for example in E- or Z-Form) or as a mixture of stereoisomers,
   with a natural or recombinant polypeptide having enal-cleaving activity, in particular a polypeptide having an α,β-unsaturated aldehyde C═C bond-cleaving, and
(2) optionally isolating the degraded product of formula IV as obtained instep (1), wherein said compound of general formula IV may be obtained in stereoisomerically pure form, or as a mixture of stereoisomers

28. The method of embodiment 27, wherein
said polypeptide having said enal-cleaving activity is selected from the group of polypeptides containing
a) at least one DUF4334 protein family domain having the Pfam ID number PF14232 (in particular within the C-terminal region of their amino acid sequence); and/or
b) at least one GXWXG protein family domain having the Pfam ID number PF14231 (in particular within the N-terminal region of their amino acid sequence); or
c) a domain retaining at least 90% sequence identity to PF14232 or PF14231:

In particular, a polypeptide of the invention having enal-cleaving activity is identified as a member of the DUF4334 protein family comprising said domain PF14232 if it matches with said domain with an e-value of less than 1×10⁻⁵, or less than 1×10⁻¹⁰, or less than 1×10⁻¹⁵, or less than 1×10⁻²⁰, or less than 1×10⁻²⁵, or less than 1×10⁻³⁰, or less than or equal to 1×10⁻³⁵, in particular in a range of 1×10⁻²⁰ to 1×10⁻³² and more particular in a range of 1×10⁻²⁵ to 1×10⁻³¹

For example, the following website may be applied for the search and calculating such e-value: pfam.xfam.org/, www.ebi.ac.uk/Tools/hmmer/search/hmmscan or www.ebi.ac.uk/Tools/pfa/pfamscan/In particular, a polypeptide of the invention having enal-cleaving activity is identified as a member of GXWXG protein family comprising said domain PF14231 if it matches with an e-value of less than 1×10⁻⁵, or less than 1×10⁻¹⁰, or less than 1×10⁻¹⁵, or less than 1×10⁻²⁰, or less than 1×10⁻²⁵, or less than 1×10⁻³⁰, or less than or equal to 1×10⁻³⁵, in particular in a range of 1×10⁻²⁰ to 1×10⁻³⁰

As the query sequence the sequence of a polypeptide having enal-cleaving activity is applied.

For example, the following website may be applied for the search and calculating such e-value: pfam.xfam.org/, www.ebi.ac.uk/Tools/hmmer/search/hmmscan or www.ebi.ac.uk/Tools/pfa/pfamscan/
and/or
wherein said polypeptide having said enal-cleaving activity is selected from the group of polypeptides selected from the group of polypeptides that comprise at least one sequence motif/domain selected from

G-[Y or “-”]-x-W-x-G-x-x-[F, L or I]-x-[T, S
or R]-G-[H or D] set forth in SEQ ID NO: 205,

or any partial motif thereof comprising up to 10 or up to 5 consecutive amino acid residues, as for example corresponding to residues in positions 1-8 or 9-13 of SEQ ID NO:205;

W-[Y, A or V]-G-K-x-[F or Y]-x-[S or D] set forth
in SEQ ID NO: 206,

or any partial motif thereof comprising up to 4 consecutive amino acid residues, as for example corresponding to residues in positions 1-4 or 5-8 of SEQ ID NO: 206;

[G or S]-x-[A or G]-x-[L or V]-x-x-x-x-[F, Y or
L]-R-G-x-V set forth in SEQ ID NO: 207,

or any partial motif thereof comprising up to 10 or up to 5 consecutive amino acid residues, as for example corresponding to residues in positions 1-8 or 9-14 of SEQ ID NO:207;

[M or L]-[V or I]-Y-D-x-x-P-[I or V]-x-D-[H or
S]-[F or L] set forth in SEQ ID NO: 208,

or any partial motif thereof comprising up to 10 or up to 5 consecutive amino acid residues, as for example corresponding to residues in positions 1-6 or 7-12 of SEQ ID NO:208:
wherein
in the above motifs residues x represent independently of each other any natural amino acid residue, and wherein optionally in each of the above motifs 1 to 5, like 1, 2, 3, 4 or 5 amino acid residues different from the x residues may be modified, for example by amino acid substitution, in particular by conservative substitutions, provided that the enzymes retains, at least to analytically detectable extent, enal-cleaving enzyme activity.
and/or
said polypeptide having said enal-cleaving activity is selected from the group consisting of the following polypeptides comprising the respective amino acid sequence:
   a) SCH94-3944 set forth in SEQ ID NO: 34
   b) SCH80-05241 set forth in SEQ ID NO:38
   c) Pdigit7033 set forth in SEQ ID NO: 42
   d) PitalDUF4334-1 set forth in SEQ ID NO: 46
   e) AspWeDUF4334 set forth in SEQ ID NO: 49
   f) RhoagDUF4334-2 set forth in SEQ ID NO: 53,
   g) RhoagDUF4334-3 set forth in SEQ ID NO: 56,
   h) RhoagDUF4334-4 set forth in SEQ ID NO: 59,
   i) CnecaDUF4334 set forth in SEQ ID NO: 62,
   j) Rins-DUF4334 set forth in SEQ ID NO: 69,
   k) CgatDUF4334 set forth in SEQ ID NO: 72,
   l) GclavDUF4334 set forth in SEQ ID NO: 75
   m) TcurvaDUF4334 set forth in SEQ ID NO:81,
   n) PprotDUF4334 set forth in SEQ ID NO: 87, and
   o) polypeptides comprising an amino acid sequence that has at least 40%, 45%, 50%, 55%, 60%, 65%, 75%, 80%, 85%, 90%, 95%, 96%, 97%, 98%, or 99% sequence identity to any one of the amino acid sequences of a) to n) and retaining said enzymatic activity of degrading an terpene precursor of formula (1).

Another particular embodiment refers to polypeptide variants of the novel polypeptides of the invention having a enal-cleaving activity as identified above by anyone of the particular amino acid sequences of SEQ ID NO: 34, 38, 42, 46, 49, 53, 56, 59, 62, 69, 72, 75, 81 and 87 and wherein the polypeptide variants are selected from an amino acid sequence having at least 70%, 75%, 80%, 85%, 90%, 95%, 96%, 97%, 98%, or 99% sequence identity to anyone of SEQ ID NO: 34, 38, 42, 46, 49, 53, 56, 59, 62, 69, 72, 75, 81 and 87, and containing at least one substitution modification relative to anyone of SEQ ID NO: 34, 38, 42, 46, 49, 53, 56, 59, 62, 69, 72, 75, 81 and 87.

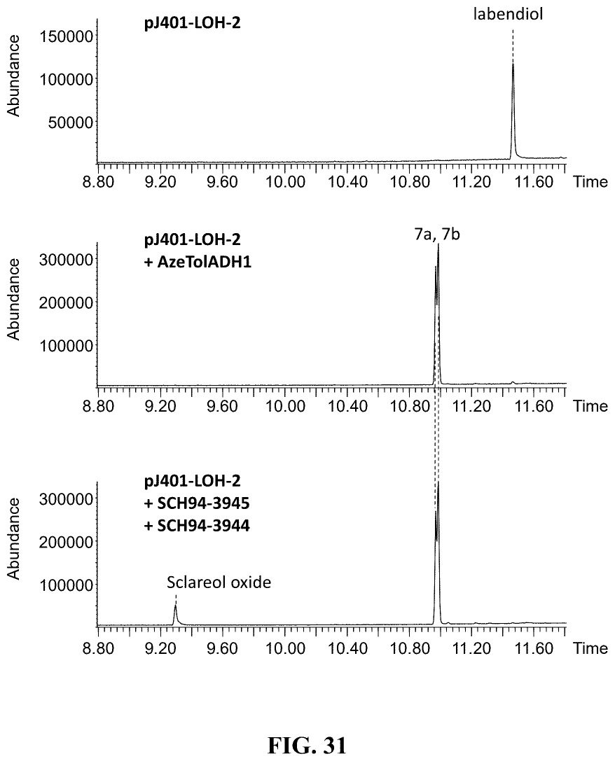

In the above referenced 14 particular enal cleaving polypeptides the protein family domains having the Pfam ID number PF14232 and PF14231 may be located at amino acid residue positions given in the following table (see also alignment depicted in FIG. 31 and the framed sequence sections therein)

| | | | | Accession | Protein |
|---|---|---|---|---|---|
| Protein sequence | From | To | E-Value | Id | domain |
| SCH94-3944 | 96 | 153 | 1.63E−30 | pf14232 | DUF4334 |
| SCH94-3944 | 27 | 85 | 3.24E−28 | pf14231 | GXWXG |
| SCH80-05241 | 96 | 153 | 7.42E−30 | pf14232 | DUF4334 |
| SCH80-05241 | 27 | 85 | 2.30E−27 | pf14231 | GXWXG |
| Pdigit7033 | 93 | 147 | 9.46E−28 | pf14232 | DUF4334 |
| Pdigit7033 | 27 | 83 | 9.73E−24 | pf14231 | GXWXG |
| PitalDUF4334-l | 93 | 147 | 1.03E−26 | pf14232 | DUF4334 |
| PitalDUF4334-l | 27 | 84 | 7.88E−25 | pf14231 | GXWXG |
| AspWeDUF4334 | 94 | 148 | 5.62E−26 | pf14232 | DUF4334 |
| AspWeDUF4334 | 27 | 85 | 6.95E−26 | pf14231 | GXWXG |
| RhoagDUF4334-2 | 94 | 150 | 8.64E−27 | pf14232 | DUF4334 |
| RhoagDUF4334-2 | 24 | 83 | 9.35E−23 | pf14231 | GXWXG |
| RhoagDUF4334-3 | 94 | 150 | 1.33E−26 | pf14232 | DUF4334 |
| RhoagDUF4334-3 | 24 | 83 | 9.55E−23 | pf14231 | GXWXG |
| RhoagDUF4334-4 | 94 | 150 | 8.31E−26 | pf14232 | DUF4334 |
| RhoagDUF4334-4 | 24 | 83 | 8.03E−23 | pf14231 | GXWXG |
| CnecaDUF4334 | 117 | 168 | 1.10E−21 | pf14232 | DUF4334 |
| CnecaDUF4334 | 20 | 75 | 1.20E−20 | pf14231 | GXWXG |
| Rins-DUF4334 | 91 | 152 | 2.57E−27 | pf14232 | DUF4334 |
| Rins-DUF4334 | 23 | 81 | 5.84E−26 | pf14231 | GXWXG |
| CgatDUF4334 | 91 | 145 | 4.15E−26 | pf14232 | DUF4334 |
| CgatDUF4334 | 24 | 82 | 8.78E−23 | pf14231 | GXWXG |
| GelavDUF4334 | 91 | 145 | 3.09E−30 | pf14232 | DUF4334 |
| GelavDUF4334 | 24 | 82 | 5.12E−29 | pf14231 | GXWXG |
| TcurvaDUF4334 | 24 | 82 | 2.85E−27 | pf14231 | GXWXG |
| TcurvaDUF4334 | 91 | 143 | 1.69E−25 | pf14232 | DUF4334 |
| PprotDUF4334 | 91 | 153 | 3.71E−27 | pf14232 | DUF4334 |
| PprotDUF4334 | 23 | 81 | 6.37E−24 | pf14231 | GXWXG |

The numbering of amino acid residues refers to the residue number in the respective SEQ ID NO of the respective protein sequence in the attached sequence listing

29. The method of embodiment 28, wherein said enal-cleaving polypeptide is selected from the following group of mutants consisting of the following polypeptides and comprising the respective amino acid sequence:
a) SCH94-3944-T51A_variant set forth in SEQ ID NO:91
b) SCH94-3944-H53A_variant set forth in SEQ ID NO:93
c) SCH94-3944-L59A_variant set forth in SEQ ID NO:95
d) SCH94-3944-W64A_variant set forth in SEQ ID NO:97
e) SCH94-3944-S71A_variant set forth in SEQ ID NO:101
f) SCH94-3944-R106A_variant set forth in SEQ ID NO: 103
g) SCH94-3944-Y115A_variant set forth in SEQ ID NO:105
h) SCH94-3944-D116A_variant set forth in SEQ ID NO:107
i) SCH94-3944-M136A_variant set forth in SEQ ID NO:111
j) SCH94-3944-K139A_variant set forth in SEQ ID NO:113
k) SCH94-3944-R156A_variant set forth in SEQ ID NO:119 and
l) polypeptides comprising an amino acid sequence that has at least 90%, 95%, 96%, 97%, 98% or 99% sequence identity to any one of the amino acid sequences of a) to 1) and retaining said enzymatic activity of degrading an terpene precursor of formula (1) and retaining said mutated amino acid sequence position.

30. The method of anyone of the embodiments 27 to 29, wherein a compound, for example a terpene-type compound, of formula V is applied, wherein
R¹ represents H or methyl,
R² represents H or
   a) a non-cyclic, linear or branched, saturated or unsaturated, hydrocarbyl residue having 1 to 20, in particular 1 to 10, 1 to 15 or 1 to 20 carbon atoms; or
   b) a group Cyc-A-, wherein A represents a straight chain or branched C₁-C₄-alkylene bridge, in particular methylene, and Cyc represents a mono- or polycyclic, in particular bicyclic, saturated or unsaturated hydrocarbyl residue, in particular a bicyclic annulated hydrocarbyl residue, comprising 5-7, in particular 6 ring atoms per cycle, optionally substituted with 1-10, 1-5 substituents which are independently selected from C₁-C₄-alkyl, C₁-C₄-alkylidene, C₂-C₄-alkenyl, oxo, hydroxy, or amino, in particular C₁-C₄-alkyl. like methyl, and C₁-C₄-alkylidene, like methylidene,
each R³ represents H,
R⁴ represents H or methyl, and
R⁵ represents H or methyl.

31 The method of embodiment 30 wherein the compound of general formula V possesses a labdane-type structure, and/or Cyc-A represents a residue of formula IIIa, IIIb or IIIc

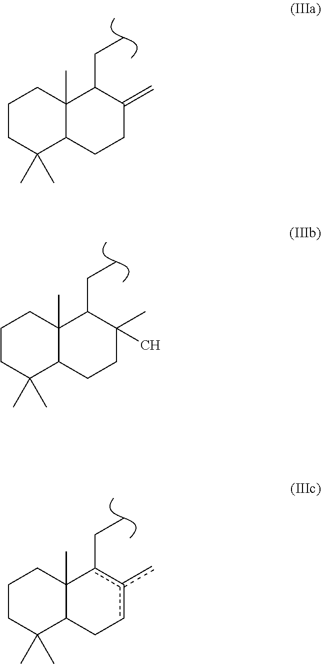

32 The method of any one of the embodiments 27 to 31, wherein the precursor of formula (V) is selected from farnesal, geranylgeranial, citral, dodecanal, labdane-type compounds, like 8-hydroxy-labd-13-en-15-al and copalal, each in the form of a mixture of its stereoisomers or in stereoisomerically pure form.

33. The method of one of the embodiments 27 to 32, wherein the degraded product of formula (IV) is selected from geranylacetone, farnesylacetone, methylheptenone, decanal; or manooloxy, or 8-hydroxy-14,15-dinorlabdan-13-one each in the form of a mixture of its stereoisomers or in stereoisomerically pure form.

Further particular inventive examples of enal-cleaving enzyme-catalyzed conversions of carbonyl compounds to the respective cleavage product are summarized in the following schematic overview:

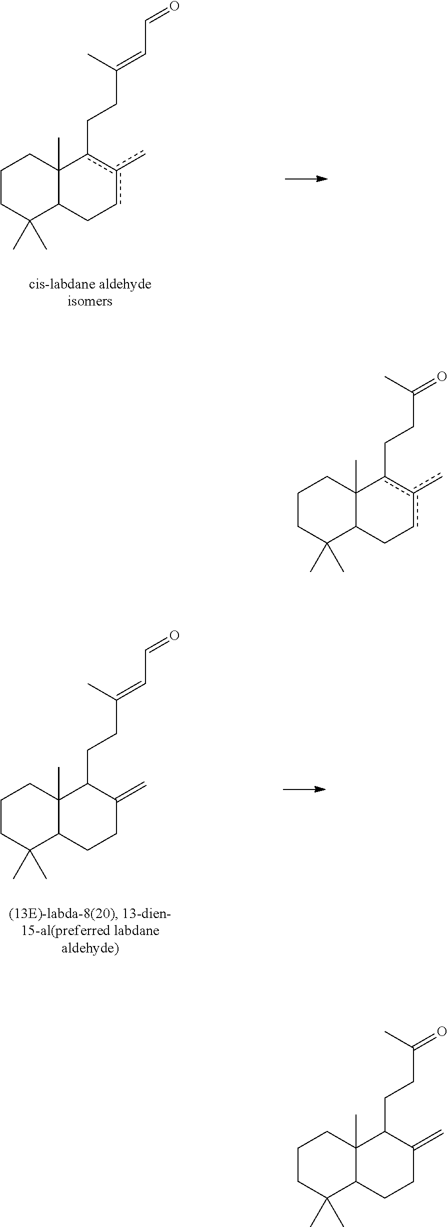

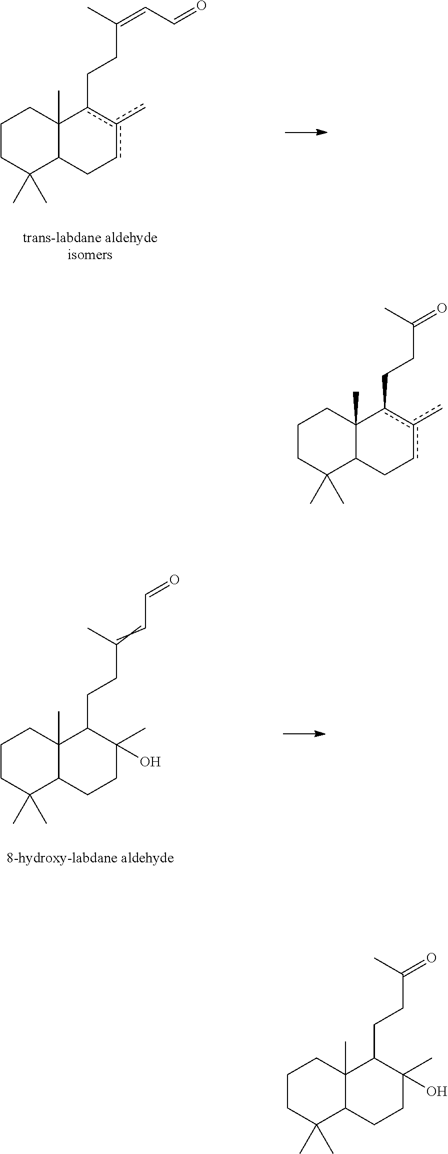

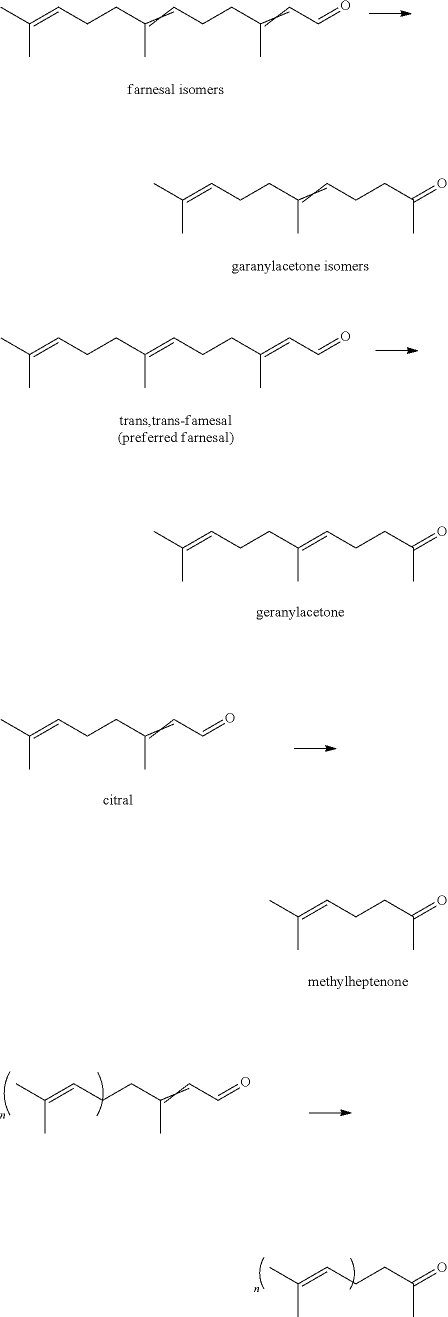

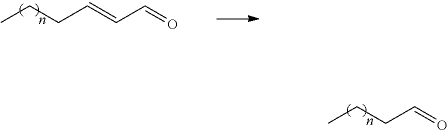

wherein parameter “n” is an integer from 1 to 20, 1 to 15, 1 to 10 or 1, 2, 3, 4 or 5.

34. The method of anyone of embodiments 27 to 33 performed in vitro or in vitro.

35. The method of embodiment 34 performed in vivo that comprises, prior to step (1), the recombinant expression, in particular in a non-human host cell, of one or more polypeptides having the enzyme activity required for performing the chain degradation step.

36. The method of embodiment 35, wherein the non-human host cell is transformed with a nucleic acid which is encoding at least one polypeptide having enal-cleaving activity.

37. The method of embodiment 35 or 36, wherein said non-human host cell is a eukaryotic or a prokaryotic cell, in particular a plant cell, a bacterial or a fungal cell, in particular a yeast cell.

38. The method of any one of embodiments 35 to 37, wherein the non-human host cell is a unicellular organism, a cultured cell derived from a multi-cellular organism, a cell present in a cultured tissue derived from a multicellular organism, or a cell present in a living multicellular organism.

39. The method of one of the embodiments 35 to 38, wherein the non-human host cell is a bacterium of the genus Escherichia, preferably E. coli and said yeast is of the genus Saccharomyces, or Pichia, preferably S. cerevisiae, or a plant cell.

40. The method of anyone of the embodiments 27 to 39 further comprising as step (3) the processing of the compound of formula IV formed in step (1) or isolated in step (2) to obtain a derivative thereof using chemical or biocatalytic synthesis or a combination of both, wherein said derivative may in particular be selected from a hydrocarbon, alcohol, diol, triol, acetal, ketal, aldehyde, acid, ether, amide, ketone, lactone, epoxide, acetate, glycoside and/or an ester and (4) optionally isolating the derivative of step (3).

41. The method of embodiment 40, wherein step (3) comprises the processing of the compound of formula IV formed in step (1) or isolated in step (2) with a polypeptide having Baeyer-Villiger monooxygenase (BVMO) activity so as to form the respective carbonyl ester.

42. The method of embodiment 41, further comprising the hydrolysis of the carbonyl ester compound with an esterase to the corresponding de-esterified product, which may be an alcohol or an isomerization product thereof, and optionally isolating the derivative of step (3).

43. The method of embodiment 41, wherein the polypeptide having BVMO activity is as defined above in embodiment 9.

44. The method of embodiment 42, wherein the esterase is selected from the group consisting of
a) polypeptides comprising the amino acid sequence of SCH23-Esterase set forth in SEQ ID NO:20;
b) polypeptides comprising the amino acid sequence of SCH24-Esterase set forth in SEQ ID NO:24;
c) polypeptides comprising the amino acid sequence of SCH25-Esterase set forth in SEQ ID NO:28;
d) polypeptides comprising the amino acid sequence of SCH46-Esterase set forth in SEQ ID NO:31; or
e) polypeptides comprising an amino acid sequence that has at least 70%, 75%, 80%, 85%, 90%, 95%, 96%, 97%, 98%, or 99%, identity to any one of the amino acid sequences of a) to d) and having esterase activity.

45. The method of embodiment 41 to 44, wherein the carbonyl compound is a dinorlabdane ketone, in particular manooloxy, which is converted by said BVMO to the respective tetranorlabdanyl acetate, in particular to gamma-ambryl acetate.

46. The method of embodiment 45, wherein tetranorlabdanyl acetate, in particular gamma-ambryl acetate is deesterified by said esterase to the respective tetranorlabdane, in particular to gamma-ambrol.

47. The method of one of the preceding embodiments 27 to 46, which method comprises prior to step (1)
the biocatalytic oxidation of a labdane alcohol to a labdane aldehyde, in particular of copalol to copalal,
which labdane alcohol is optionally formed by the biocatalytic conversion of at least one terpenly diphosphate precursor, selected from IPP, DMAPP, FPP and GGPP, in particular in a single step or a combination of at least two steps, known in the prior art.

Said labdane alcohol may for example be biocatalytically produced:
a) from geranylgeranyl diphosphate (GGPP) in one step in a cyclisation reaction/dephosphorylation reaction
b) from GGPP in two steps by a cyclisation forming labdane diphosphate, as for example copalyl diphosphate (CPP) which is then dephosphorylated to the labdane alcohol;
c) from IPP and DMAPP which is directly converted through the action of a bifunctional GGPP synthase/CPP synthase to the labdane diphosphate, as for example CPP which is then dephosphorylated;
GGPP as used in these steps may also be provided by different biocatalytic steps:
d) GGPP synthases are available which produce GGPP directly from IPP and DMAPP; or
e) GGPP may be provided from IPP and DMAPP via FPP through the action of a FPP synthase, and the subsequent conversion of FPP to GGPP through the action of a GGPP synthase.

48. The method of embodiment 47, wherein
said biocatalytic oxidation of a labdane alcohol, in particular of copalol to copalal, is catalyzed by an exogenous or endogenous polypeptide having alcohol dehydrogenase (ADH) (EC 1.1.1.-) activity; and/or said biocatalytic formation of the labdane alcohol comprises at least one step selected from
i) a biocatalytic dephosphorylation of a labdane diphosphate to a labdane aldehyde, in particular of copalyl diphosphate (CPP) to copalol, which is catalyzed by a polypeptide having terpenyl diphosphate (TPP) phosphatase activity, and/or
ii) a biocatalytic cyclisation of a terpenly diphosphate precursor, as for example of geranylgeranyl diphosphate (GGPP) to CPP, which is catalyzed by a polypeptide having CPP synthase activity, like SmCPS2 (SEQ ID NO: 185); or as for example of IPP and DMAPP to CPP, which is catalyzed by a bifunctional polypeptide having prenyl-transferase and copalyl-diphosphate synthase activity, like PvCPS, and/or
iii) a biocatalytic formation of GGPP from FPP or a biocatalytic formation from IPP and DMAPP, each of which being catalyzed by a polypeptide having GGPP synthase activity.

49. The method of embodiment 48, wherein
said biocatalytic oxidation, in particular of copalol to copalal, is catalyzed by a polypeptide having alcohol dehydrogenase (ADH) activity selected from
a) polypeptides comprising the amino acid sequence of SCH23-ADH1_wt set forth in SEQ ID NO: 134
b) polypeptides comprising the amino acid sequence of SCH24-ADH1_wt set forth in SEQ ID NO: 140
c) polypeptides comprising the amino acid sequence of SCH94-3945_wt set forth in SEQ ID NO:161
d) polypeptides comprising the amino acid sequence of SCH80-0540_wt set forth in SEQ ID NO:164
e) polypeptides comprising the amino acid sequence of AzTolADH1_wt set forth in SEQ ID NO:167
f) polypeptides comprising the amino acid sequence of CdGeoA_wt set forth in SEQ ID NO: 179
g) polypeptides comprising an amino acid sequence that has at least 70%, 75%, 80%, 85%, 90%, 95%, 96%, 97%, 98%, or 99% identity to any one of the amino acid sequences of a) to f) and having ADH activity.
and/or
said biocatalytic dephosphorylation, in particular of copalyl diphosphate (CPP) to copalol, is catalyzed by a polypeptide having terpenyl diphosphate (TPP) phosphatase activity selected from
d) polypeptides comprising an amino acid sequence of AspWE TPP as set forth in SEQ ID NO:170 or a polypeptide comprising an amino acid sequence that has at least 70%, 75%, 80%, 85%, 90%, 95%, 96%, 97%, 98%, or 99% identity thereto;
e) polypeptides comprising an amino acid sequence of TalCeTPP as set forth in SEQ ID NO: 176 or a polypeptide comprising an amino acid sequence that has at least 70%, 75%, 80%, 85%, 90%, 95%, 96%, 97%, 98%, or 99% identity thereto; and
f) polypeptides comprising an amino acid sequence of TalVeTPP as set forth in SEQ ID NO: 194 or a polypeptide comprising an amino acid sequence that has at least 70%, 75%, 80%, 85%, 90%, 95%, 96%, 97%, 98%, or 99% identity thereto;

Further suitable phosphatases are also disclosed in earlier the applicant's EP Application number 18182783.3, incorporated by reference.
and/or
said biocatalytic cyclisation, in particular of geranylgeranyl diphosphate (GGPP) to CPP, is catalyzed by a polypeptide selected from
   polypeptides having copalyl-diphosphate synthase activity comprising the amino acid sequence of SmCPS2 as set forth in SEQ ID NO:185 or a polypeptide comprising an amino acid sequence that has at least 70%, 75%, 80%, 85%, 90%, 95%, 96%, 97%, 98%, or 99% 70% identity thereto;
said biocatalytic cyclisation, in particular of IPP and DNMAPP to CPP, is catalyzed by
a polypeptide selected from
   polypeptides having prenyl-transferase and copalyl-diphosphate synthase activities comprising the amino acid sequence of PvCPS as set forth in SEQ ID NO: 173 or a polypeptide comprising an amino acid sequence that has at least 70%, 75%, 80%, 85%, 90%, 95%, 96%, 97%, 98%, or 99% identity thereto;
   and/or
said biocatalytic formation of GGPP is catalyzed by a polypeptide having GGPP synthase activity and is selected from
d) polypeptides comprising the amino acid sequence of carG as set forth in SEQ ID NO: 182 or a polypeptide comprising an amino acid sequence that has at least 70%, 75%, 80%, 85%, 90%, 95%, 96%, 97%, 98%, or 99% identity thereto;
e) polypeptides comprising the amino acid sequence of CrtE as set forth in SEQ ID NO: 191 or a polypeptide comprising an amino acid sequence that has at least 70%, 75%, 80%, 85%, 90%, 95%, 96%, 97%, 98%, or 99% identity thereto;
f) polypeptides comprising the amino acid sequence of PvCPS as set forth in SEQ ID NO: 173 or a polypeptide comprising an amino acid sequence that has at least 70%, 75%, 80%, 85%, 90%, 95%, 96%, 97%, 98%, or 99% identity thereto.

iii) The present invention relates to the following particular embodiments related to enal-cleaving enzymes and corresponding coding sequences

50. An isolated polypeptide having enal-cleaving activity in particular the activity of an α,β-unsaturated aldehyde C═C bond-cleaving enzyme, as defined in anyone of the embodiments 28 and 29.

The polypeptides of the invention include all active forms, including active subsequences, e.g., catalytic domains or active sites, of an enzyme with enal cleaving activity.

51. An isolated nucleic acid molecule comprising a nucleic acid sequence encoding a polypeptide of embodiment 50 in particular a nucleic acid sequence selected from SEQ ID NOs: 33, 35, 36, 37, 39, 40, 41, 43, 44, 45, 47, 48, 50, 51, 52, 54, 55, 57, 58, 60, 61, 63, 64, 68, 70, 71, 73, 74, 76, 80, 82, 86, 88, 92, 94, 96, 98, 102, 104, 106, 108, 112, and 120, and nucleic acid sequences having a degree of sequence identity of at least 70%, 75%, 80%, 85%, 90%, 95%, 96%, 97%, 98%, or 99% to any one of said sequences of SEQ ID NO: 33, 35, 36, 37, 39, 40, 41, 43, 44, 45, 47, 48, 50, 51, 52, 54, 55, 57, 58, 60, 61, 63, 64, 68, 70, 71, 73, 74, 76, 80, 82, 86, 88, 92, 94, 96, 98, 102, 104, 106, 108, 112, and 120.

52. An expression cassette comprising the nucleotide sequence of at least one nucleic acid molecule of embodiment 50.

53. An expression vector comprising the nucleotide sequence of at least one nucleic acid molecule of embodiment 51, or at least one expression cassette of embodiment 52.

54. The expression vector of embodiment 53, wherein the vector is a prokaryotic vector, viral vector or eukaryotic vector.

55. The expression vector of anyone of the embodiments 53 to 54, which is a plasmid or a combination of two or more plasmids.

56. A recombinant non-human host cell comprising at least one nucleic acid molecule as defined in embodiment 51, or at least one expression cassette of embodiment 52, or at least one expression vector of any one of embodiments 53 to 55.

57. The host cell of embodiment 56, wherein the at least one nucleic acid molecule or the at least one expression cassette is stably integrated into the genome of the cell.

58. The host cell of embodiment 56 or 57 which is a prokaryotic or eukaryotic cell, in particular a plant cell, a bacterium or a fungal cell, in particular a yeast.

59. The host cell of anyone of the embodiments 56 to 58 which is a unicellular organism, a cultured cell derived from a multi-cellular organism, a cell present in a cultured tissue derived from a multicellular organism, or a cell present in a living multicellular organism.

60. The host cell of embodiment 59 which is a bacterium of the genus Escherichia, preferably E. coli, or a yeast cells of the genus Saccharomyces, preferably S. cerevisiae, or of the genus Pichia, preferably P. pastoris.

61. A method of producing at least one polypeptide having enal-cleaving activity according to embodiment 51, the method comprising:
(i) expressing said at least one polypeptide in a non-human host cell of any one of embodiments 57 to 60; and
(ii) optionally isolating said at least one polypeptide from the non-human host cell used in step (i).

62. The method of embodiment 61 further comprising, prior to step (i): preparing the non-human host cell used in step (i) by introducing at least on nucleic acid molecule as defined in embodiment 51, or at least one expression cassette of embodiment 52, or at least one expression vector of any one of embodiments 53 to 55 into a non-human cell, thus yielding a host cell capable of expressing or over-expressing the at least one polypeptide having enal cleaving activity according to embodiment 50.

63. A method for preparing a mutant polypeptide having enal-cleaving activity, which method comprises the steps of:
(i) providing a nucleic acid molecule according to embodiment 51;
(ii) modifying the nucleotide sequence of said nucleic acid molecule, in particular the nucleotide sequence encoding a polypeptide of embodiment 50, so as to obtain at least one mutant nucleic acid molecule;
(iii) recombinantly expressing said mutant nucleic acid molecule in a non-human host cell;
(iv) screening the expression product obtained in step (iii) for at least one mutant polypeptide having enal cleaving activity; and
(v) optionally repeating steps (ii) to (iv) with the mutant nucleic acid molecule until the expression product comprises a mutant polypeptide having the desired enal cleaving activity; and
(vi) optionally isolating the mutant polypeptide having the desired enal cleaving activity.

iv) The present invention relates to the following particular embodiments related to BVMO enzymes and corresponding coding sequences

64. An isolated polypeptide having BVMO activity, as defined in embodiment 9.

The polypeptides of the invention include all active forms, including active subsequences, e.g., catalytic domains or active sites, of an enzyme with BVMO activity.

65. An isolated nucleic acid molecule comprising a nucleic acid sequence encoding a polypeptide of embodiment 64, in particular a nucleic acid sequence seleted from SEQ ID NOs: 1, 3, 4, 5, 7, 8, 9, 11, 12, 14, 15, 17 and 18 and nucleic acid sequences having a degree of sequence identity of at least 70%, 75%, 80%, 85%, 90%, 95%, 96%, 97%, 98%, or 99% to any one of said sequences of SEQ ID NO: 1, 3, 4, 5, 7, 8, 9, 11, 12, 14, 15, 17 and 18.

66. An expression cassette comprising the nucleotide sequence of at least one nucleic acid molecule of embodiment 65.

67. An expression vector comprising the nucleotide sequence of at least one nucleic acid molecule of embodiment 65, or at least one expression cassette of embodiment 66.

68. The expression vector of embodiment 67, wherein the vector is a prokaryotic vector, viral vector or eukaryotic vector.

69. The expression vector of anyone of the embodiments 67 to 68, which is a plasmid or a combination of two or more plasmids.

70. A recombinant non-human host cell comprising at least one nucleic acid molecule as defined in embodiment 65, or at least one expression cassette of embodiment 66, or at least one expression vector of any one of embodiments 67 to 69.

71. The host cell of embodiment 70, wherein the at least one nucleic acid molecule or the at least one expression cassette is stably integrated into the genome of the cell.

72. The host cell of embodiment 70 or 71 which is a prokaryotic or eukaryotic cell, in particular a plant cell, a bacterium or a fungal cell, in particular a yeast.

73. The host cell of anyone of the embodiments 70 to 72 which is a unicellular organism, a cultured cell derived from a multi-cellular organism, a cell present in a cultured tissue derived from a multicellular organism, or a cell present in a living multicellular organism.

74. The host cell of embodiment 72 which is a bacterium of the genus Escherichia, preferably E. coli, or a yeast cells of the genus Saccharomyces, preferably S. cerevisiae, or of the genus Pichia, preferably P. pastoris.

75. A method of producing at least one polypeptide having BVMO activity according to embodiment 64, the method comprising:
(i) expressing said at least one polypeptide in a non-human host cell of any one of embodiments 70 to 74; and
(ii) optionally isolating said at least one polypeptide from the non-human host cell used in step (i).

76. The method of embodiment 75 further comprising, prior to step (i): preparing the non-human host cell used in step (i) by introducing at least on nucleic acid molecule as defined in embodiment 65, or at least one expression cassette of embodiment 66, or at least one expression vector of any one of embodiments 67 to 69 into a non-human cell, thus yielding a host cell capable of expressing or over-expressing the at least one polypeptide having BVMO activity according to embodiment 64.

77. A method for preparing a mutant polypeptide having BVMO activity, which method comprises the steps of:
(i) providing a nucleic acid molecule according to embodiment 65;
(ii) modifying the nucleotide sequence of said nucleic acid molecule, in particular the nucleotide sequence encoding a polypeptide of embodiment 64, so as to obtain at least one mutant nucleic acid molecule;
(iii) recombinantly expressing said mutant nucleic acid molecule in a non-human host cell;
(iv) screening the expression product obtained in step (iii) for at least one mutant polypeptide having BVMO activity; and
(v) optionally repeating steps (ii) to (iv) with the mutant nucleic acid molecule until the expression product comprises a mutant polypeptide having the desired BVMO activity; and
(vi) optionally isolating the mutant polypeptide having the desired BVMO activity.

v) The present invention relates to the following particular embodiments related to biocatalytic multistep in vivo methods of converting labdane compounds by applying polypeptides with enal-cleaving activity and/or BVMO activity

78. An in vivo method for preparing labdane-type terpenes
which method comprises providing a recombinant host expressing a set of polypeptides having enzymatic activities required for catalyzing the following sequence of reaction steps
(1) optionally converting a labdane alcohol, in particular a copalol, to the respective labdane aldehyde, in particular a copalal, through the enzymatic action of an exogenous or endogenous ADH polypeptide, in particular an ADH as defined in anyone of the embodiments 19 or 49;
(2) converting said ladbane aldehyde of step (1), in particular a copalal, to the respective dinorlabdane carbonyl compound, in particular manooloxy, through the action a polypeptide having enal-cleaving activity, in particular a polypeptide as defined in anyone of the embodiments 28 and 29;
(3) optionally converting said dinorlabdane carbonyl compound of step (2), in particular manooloxy, to the respective tetranorlabdanyl acetate, in particular to gamma-ambryl acetate through the action a polypeptide having BVMO activity, in particular BVMO as defined in embodiment 9;
(4) optionally converting said tetranorlabdanyl acetate of step (3), in particular to gamma-ambryl acetate, to the respective tetranorlabdane alcohol, in particular gamma, ambrol, through the action a polypeptide having esterase activity, in particular an esterase as defined in anyone of the embodiment 23 and 44; and optionally
(5) isolating the product of step (2), (3) or (4).

79. An in vivo method for preparing labdane-type cyclo-terpenes which method comprises providing a recombinant host expressing a set of polypeptides having enzymatic activities required for catalyzing the following sequence of reaction steps
(1) optionally converting a labdane alcohol, in particular a copalol, to the respective labdane aldehyde, in particular a copalal, through the enzymatic action of an exogenous or endogenous ADH polypeptide, in particular an ADH as defined in anyone of the embodiments 19 or 49;
(2) converting said labdane aldehyde of step (1), in particular a copalal, to the respective norlabdane ester compound, in particular [4-[(1S,4aS,8aS)-5,5,8a-trimethyl-2-methylene-decalin-1-yl]-2-methyl-but-1-enyl] formate (compound 1a,1b), through the action a polypeptide having BVMO activity, in particular a BVMO as defined in anyone of the embodiments 9;
(3) converting said labdane ester compound of step (2), in particular in particular [4-[(1S,4aS,8aS)-5,5,8a-trimethyl-2-methylene-decalin-1-yl]-2-methyl-but-1-enyl] formate (compound 1a,1b) to the respective norlabdane aldehyde, in particular 4-[(1S,8aS)-5,5,8a-trimethyl-2-methylene-decalin-1-yl]-2-methyl-butanal (compound 3a,3b), optionally through the action a polypeptide having esterase activity, in particular an esterase as defined in anyone of the embodiments 23 or 44;
(4) converting said norlabdane aldehyde of step (3), in particular 4-[(1S,4aS,8aS)-5,5,8a-trimethyl-2-methylene-decalin-1-yl]-2-methyl-butanal (compound 3a,3b), to the respective dinorlabdane ester, in particular [3-[(1S,4aS,8aS)-5,5,8a-trimethyl-2-methylene-decalin-1-yl]-1-methyl-propyl] formate (compound 4a,4b), through the action a polypeptide having BVMO activity, in particular a BVMO as defined in anyone of the embodiments 9;
(5) converting said dinorlabdane ester of step (4) in particular [3-[(1S,4aS,8aS)-5,5,8a-trimethyl-2-methylene-decalin-1-yl]-1-methyl-propyl] formate (compound 4a,4b), to the respective dinorlabdane alcohol, in particular 4-[(1S,4aS,8aS)-5,5,8a-trimethyl-2-methylene-decalin-1-yl]butan-2-ol, (compound 5a,5b), through the action a polypeptide having esterase activity, in particular an esterase as defined in anyone of the embodiments 23 or 44;
(6) optionally converting said dinorlabdane alcohol of step (5), in particular 4-[(1S,8aS)-5,5,8a-trimethyl-2-methylene-decalin-1-yl]butan-2-ol, (compound 5a,5b), to the respective dinorlabdane carbonyl compound, in particular manooloxy, through the action an exogenous or endogenous polypeptide having ADH activity, in particular an ADH as defined in anyone of the embodiments 19 or 49;
(7) optionally converting said dinorlabdane carbonyl compound of step (6), in particular manooloxy, to the respective tetranorlabdanyl acetate, in particular to gamma-ambryl acetate through the action a polypeptide having BVMO activity, in particular a BVMO as defined in anyone of the embodiments 9;
(8) converting said tetranorlabdanyl acetate of step (7), in particular to gamma-ambryl acetate, to the respective tetranorlabdane alcohol, in particular gamma, ambrol through the action a polypeptide having esterase activity, in particular an esterase as defined in anyone of the embodiments 23 or 44; and optionally
(9) isolating the product of step (5), (6), (7) or (8).

80. The method of embodiment 79, wherein
the ADHs as applied in steps (1) and (6) are identical or different; exogenous or endogenous, and/or
the BVMOs as applied in steps (2), (4) and (7) are identical or different; and/or the esterases as applied in steps (3), (5) and (8) are identical or different.

81. The method of anyone of the embodiments 78 to 80, wherein
a recombinant host is applied additionally expressing a set of polypeptides having enzymatic activities required for catalyzing the following sequence of reaction steps in advance of step (1):
(i) the biocatalytic formation of geranylgeranyl diphosphate (GGPP) through the action a polypeptide having GGPP synthase activity, in particular a GGPP synthase as defined in anyone of the embodiments 19 and 49;
(ii) the biocatalytic cyclisation of GGPP to said labdane diphosphate, in particular to a copalyl diphosphate (CPP) through the action a polypeptide having labdane diphosphate synthase activity, in particular a polypeptide comprising CPP synthase activity as defined in anyone of embodiments 19 and 49;
(iii) the biocatalytic dephosphorylation of said labdane diphosphate to said labdane alcohol, in particular of CPP to copalol, through the action a polypeptide having labdane diphosphate phosphatase activity, in particular a polypeptide comprising TPP phosphatase activity as defined in anyone of embodiments 19 and 49.

82 The method of anyone of the embodiments 78 to 81, wherein a recombinant host is applied additionally expressing at least one of the polypeptide catalyzing an enzymatic step of the mevalonate pathway or the MEP pathway.

83. The method of one of the embodiments 78 to 82 wherein the a recombinant host is applied which carries the coding sequences of the respective catalytically active polypeptides on one or more expression vectors and/or stably integrated into the genome of the host.

84. The method of anyone of the embodiments 1 to 49 and 78 to 83 performed in vivo, which comprises prior to step (1) introducing into a non-human host organism or cell and optionally stably integrated into the respective genome: one or more nucleic acid molecules encoding one or more polypeptides having the enzyme activities required for performing the respective biocatalytic conversion step or steps.

85. The method of anyone of the embodiments 1 to 49 and 78 to 83 performed by applying a non-human host organism or cell endogenously producing FPP and/or GGPP; or a mixture of IPP and DMAPP; or a non-human host organism which is genetically modified to produce increased amounts of FPP and/or of GGPP and/or of a mixture of IPP and DMAPP.

Some of these host cells or organisms applicable in the invention do not produce FPP or GGPP or a mixture of IPP and DMAPP naturally. Such organisms or cells that do not produce an acyclic terpene pyrophosphate precursor, e.g. FPP or GGPP or a mixture of IPP and DMAPP, naturally may be genetically modified to produce said precursor. They can be, for example, so transformed either before the modification with nucleic acids described herein. Methods to transform organisms so that they produce an acyclic terpene pyrophosphate precursor, e.g. FPP or GGPP or a mixture of IPP and DMAPP, are already known in the art. For example, introducing enzyme activities of the mevalonate pathway, is a suitable strategy to make the organism produce FPP or GGPP or a mixture of IPP and DMAPP.

86. The recombinant microorganism as defined in anyone of the embodiments 78 to 85.

vi) The present invention relates to the following particular embodiments related to the further conversion of chemical intermediate compounds as obtained by a biocatalytic method described herein to further final products of particular interest

87. A method of preparing an epoxy-tetranorlabdane compound, in particular ambrox, which method comprises
(1) providing a tetranorlabdane alcohol, in particular gamma-ambrol, or a tetranorlabdane acetate, in particular gamma-ambryl acetate, or a dinorlabdane carbonyl compound, in particular manooloxy, by applying a biocatalytic method comprising one or more method steps as defined in anyone of the claims 1 to 49 or 78 to 83, optionally isolating said product; and
(2) converting said product of step (1) to epoxy-tetranorlabdane in particular ambrox, by applying one or more chemical and/or biochemical conversion steps.

88. A method of preparing a diepoxy-dinorlabdabe, in particular Z11, which method comprises
(1) providing a dinorlabdane carbonyl compound, in particular manooloxy by applying a method which results in the formation of said dinorlabdane carbonyl compound, in particular manooloxy and which comprising one or more method steps as defined in anyone of the claims 1 to 49 or 78 to 84, optionally isolating said dinorlabdane carbonyl compound, in particular manooloxy; and
(2) converting said dinorlabdane carbonyl compound, in particular manooloxy of step (1) to said diepoxy-dinorlabdabe, in particular Z-11, by applying one or more chemical and/or biochemical conversion steps.

### b. Polypeptides Applicable According to the Invention

In this context the following definitions apply:

The generic terms “polypeptide” or “peptide”, which may be used interchangeably, refer to a natural or synthetic linear chain or sequence of consecutive, peptidically linked amino acid residues, comprising about 10 to up to more than 1.000 residues. Short chain polypeptides with up to 30 residues are also designated as “oligopeptides”.

The term “protein” refers to a macromolecular structure consisting of one or more polypeptides. The amino acid sequence of its polypeptide(s) represents the “primary structure” of the protein. The amino acid sequence also predetermines the “secondary structure” of the protein by the formation of special structural elements, such as alpha-helical and beta-sheet structures formed within a polypeptide chain. The arrangement of a plurality of such secondary structural elements defines the “tertiary structure” or spatial arrangement of the protein. If a protein comprises more than one polypeptide chains said chains are spatially arranged forming the “quaternary structure” of the protein. A correct spacial arrangement or “folding” of the protein is prerequisite of protein function. Denaturation or unfolding destroys protein function. If such destruction is reversible, protein function may be restored by refolding.

A typical protein function referred to herein is an “enzyme function”, i.e, the protein acts as biocatalyst on a substrate, for example a chemical compound, and catalyzes the conversion of said substrate to a product. An enzyme may show a high or low degree of substrate and/or product specificity.

A “polypeptide” referred to herein as having a particular “activity” thus implicitly refers to a correctly folded protein showing the indicated activity, as for example a specific enzyme activity.

Thus, unless otherwise indicated the term “polypeptide” also encompasses the terms “protein” and “enzyme”.

Similarly, the term “polypeptide fragment” encompasses the terms “protein fragment” and “enzyme fragment”.

The term “isolated polypeptide” refers to an amino acid sequence that is removed from its natural environment by any method or combination of methods known in the art and includes recombinant, biochemical and synthetic methods.

“Target peptide” refers to an amino acid sequence which targets a protein, or polypeptide to intracellular organelles, i.e., mitochondria, or plastids, or to the extracellular space (secretion signal peptide). A nucleic acid sequence encoding a target peptide may be fused to the nucleic acid sequence encoding the amino terminal end, e.g., N-terminal end, of the protein or polypeptide, or may be used to replace a native targeting polypeptide.

The present invention also relates to “functional equivalents” (also designated as “analogs” or “functional mutations”) of the polypeptides specifically described herein.

For example, “functional equivalents” refer to polypeptides which, in a test used for determining enzymatic terpenyl diphosphate synthase activity, or terpenyl diphosphate phosphatase activity display at least a 1 to 10%, or at least 20%, or at least 50%, or at least 75%, or at least 90% higher or lower activity, as that of the polypeptides specifically described herein.

“Functional equivalents”, according to the invention, also cover particular mutants, which, in at least one sequence position of an amino acid sequences stated herein, have an amino acid that is different from that concretely stated one, but nevertheless possess one of the aforementioned biological activities, as for example enzyme activity. “Functional equivalents” thus comprise mutants obtainable by one or more, like 1 to 20, in particular 1 to 15 or 5 to 10 amino acid additions, substitutions, in particular conservative substitutions, deletions and/or inversions, where the stated changes can occur in any sequence position, provided they lead to a mutant with the profile of properties according to the invention. Functional equivalence is in particular also provided if the activity patterns coincide qualitatively between the mutant and the unchanged polypeptide, i.e. if, for example, interaction with the same agonist or antagonist or substrate, however at a different rate, (i.e. expressed by a EC₅₀ or IC₅₀ value or any other parameter suitable in the present technical field) is observed. Examples of suitable (conservative) amino acid substitutions are shown in the following table:

| Original | Examples of |
|---|---|
| residue | substitution |
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described herein, as well as “functional derivatives” and “salts” of the polypeptides.

“Precursors” are in that case natural or synthetic precursors of the polypeptides with or without the desired biological activity.

The expression “salts” means salts of carboxyl groups as well as salts of acid addition of amino groups of the protein molecules according to the invention. Salts of carboxyl groups can be produced in a known way and comprise inorganic salts, for example sodium, calcium, ammonium, iron and zinc salts, and salts with organic bases, for example amines, such as triethanolamine, arginine, lysine, piperidine and the like. Salts of acid addition, for example salts with inorganic acids, such as hydrochloric acid or sulfuric acid and salts with organic acids, such as acetic acid and oxalic acid, are also covered by the invention.

“Functional derivatives” of polypeptides according to the invention can also be produced on functional amino acid side groups or at their N-terminal or C-terminal end using known techniques. Such derivatives comprise for example aliphatic esters of carboxylic acid groups, amides of carboxylic acid groups, obtainable by reaction with ammonia or with a primary or secondary amine: N-acyl derivatives of free amino groups, produced by reaction with acyl groups; or O-acyl derivatives of free hydroxyl groups, produced by reaction with acyl groups.

“Functional equivalents” naturally also comprise polypeptides that can be obtained from other organisms, as well as naturally occurring variants. For example, areas of homologous sequence regions can be established by sequence comparison, and equivalent polypeptides can be determined on the basis of the concrete parameters of the invention.

“Functional equivalents” also comprise “fragments”, like individual domains or sequence motifs, of the polypeptides according to the invention, or N- and or C-terminally truncated forms, which may or may not display the desired biological function. Preferably such “fragments” retain the desired biological function at least qualitatively.

“Functional equivalents” are, moreover, fusion proteins, which have one of the polypeptide sequences stated herein or functional equivalents derived there from and at least one further, functionally different, heterologous sequence in functional N-terminal or C-terminal association (i.e. without substantial mutual functional impairment of the fusion protein parts). Non-limiting examples of these heterologous sequences are e.g. signal peptides, histidine anchors or enzymes.

“Functional equivalents” which are also comprised in accordance with the invention are homologs to the specifically disclosed polypeptides. These have at least 60%, preferably at least 75%, in particular at least 80 or 85%, such as, for example, 90, 91, 92, 93, 94, 95, 96, 97, 98 or 99%, homology (or identity) to one of the specifically disclosed amino acid sequences, calculated by the algorithm of Pearson and Lipman, Proc. Natl. Acad, Sci. (USA) 85 (8), 1988, 2444-2448. A homology or identity, expressed as a percentage, of a homologous polypeptide according to the invention means in particular an identity, expressed as a percentage, of the amino acid residues based on the total length of one of the amino acid sequences described specifically herein.

The identity data, expressed as a percentage, may also be determined with the aid of BLAST alignments, algorithm blastp (protein-protein BLAST), or by applying the Clustal settings specified herein below.

In the case of a possible protein glycosylation, “functional equivalents” according to the invention comprise polypeptides as described herein in deglycosylated or glycosylated form as well as modified forms that can be obtained by altering the glycosylation pattern.

Functional equivalents or homologues of the polypeptides according to the invention can be produced by mutagenesis, e.g. by point mutation, lengthening or shortening of the protein or as described in more detail below.

Functional equivalents or homologs of the polypeptides according to the invention can be identified by screening combinatorial databases of mutants, for example shortening mutants. For example, a variegated database of protein variants can be produced by combinatorial mutagenesis at the nucleic acid level, e.g. by enzymatic ligation of a mixture of synthetic oligonucleotides. There are a great many methods that can be used for the production of databases of potential homologues from a degenerated oligonucleotide sequence. Chemical synthesis of a degenerated gene sequence can be carried out in an automatic DNA synthesizer, and the synthetic gene can then be ligated in a suitable expression vector. The use of a degenerated genome makes it possible to supply all sequences in a mixture, which code for the desired set of potential protein sequences. Methods of synthesis of degenerated oligonucleotides are known to a person skilled in the art.

In the prior art, several techniques are known for the screening of gene products of combinatorial databases, which were produced by point mutations or shortening, and for the screening of cDNA libraries for gene products with a selected property. These techniques can be adapted for the rapid screening of the gene banks that were produced by combinatorial mutagenesis of homologues according to the invention. The techniques most frequently used for the screening of large gene banks, which are based on a high-throughput analysis, comprise cloning of the gene bank in expression vectors that can be replicated, transformation of the suitable cells with the resultant vector database and expression of the combinatorial genes in conditions in which detection of the desired activity facilitates isolation of the vector that codes for the gene whose product was detected. Recursive Ensemble Mutagenesis (REM), a technique that increases the frequency of functional mutants in the databases, can be used in combination with the screening tests, in order to identify homologues.

An embodiment provided herein provides orthologs and paralogs of polypeptides disclosed herein as well as methods for identifying and isolating such orthologs and paralogs. A definition of the terms “ortholog” and “paralog” is given below and applies to amino acid and nucleic acid sequences.

The polypeptides of the invention include all active forms, including active subsequences. e.g., catalytic domains or active sites, of an enzyme of the invention. In one aspect, the invention provides catalytic domains or active sites as set forth below. In one aspect, the invention provides a peptide or polypeptide comprising or consisting of an active site domain as predicted through use of a database such as Pfam (pfam.wustl.edu/hmmsearch.shtml) (which is a large collection of multiple sequence alignments and hidden Markov models covering many common protein families, The Pfam protein families database, A. Bateman, E. Birney, L. Cerruti, R. Durbin, L. Etwiller, S. R. Eddy, S. Griffiths-Jones, K. L. Howe, M. Marshall, and E. L. L. Sonnhammer, Nucleic Acids Research, 30 (1): 276-280, 2002) or equivalent, as for example InterPro and SMART databases (www.ebi.ac.uk/interpro/scan.html, smart.embl-heidelberg.de/).

The invention also encompasses “polypeptide variant” having the desired activity, wherein the variant polypeptide is selected from an amino acid sequence having at least 40%, 45%, 50%, 55%, 60%, 65%, 70%, 75%, 80%, 85%, 90%, 91%, 92%, 93%, 94%, 95%, 96%, 97%, 98% or 99%, sequence identity to a specific, in particular natural, amino acid sequence as referred to by a specific SEQ ID NO and contains at least one substitution modification relative said SEQ ID NO.

### c. Coding Nucleic Acid Sequences Applicable According to the Invention

In this context the following definitions apply:

The terms “nucleic acid sequence,” “nucleic acid,” “nucleic acid molecule” and “polynucleotide” are used interchangeably meaning a sequence of nucleotides. A nucleic acid sequence may be a single-stranded or double-stranded deoxyribonucleotide, or ribonucleotide of any length, and include coding and non-coding sequences of a gene, exons, introns, sense and anti-sense complimentary sequences, genomic DNA, cDNA, miRNA, siRNA, mRNA, rRNA, IRNA, recombinant nucleic acid sequences, isolated and purified naturally occurring DNA and/or RNA sequences, synthetic DNA and RNA sequences, fragments, primers and nucleic acid probes. The skilled artisan is aware that the nucleic acid sequences of RNA are identical to the DNA sequences with the difference of thymine (T) being replaced by uracil (U). The term “nucleotide sequence” should also be understood as comprising a polynucleotide molecule or an oligonucleotide molecule in the form of a separate fragment or as a component of a larger nucleic acid.

An “isolated nucleic acid” or “isolated nucleic acid sequence” relates to a nucleic acid or nucleic acid sequence that is in an environment different from that in which the nucleic acid or nucleic acid sequence naturally occurs and can include those that are substantially free from contaminating endogenous material.

The term “naturally-occurring” as used herein as applied to a nucleic acid refers to a nucleic acid that is found in a cell of an organism in nature and which has not been intentionally modified by a human in the laboratory.

A “fragment” of a polynucleotide or nucleic acid sequence refers to contiguous nucleotides that is particularly at least 15 bp, at least 30 bp, at least 40 bp, at least 50 bp and/or at least 60 bp in length of the polynucleotide of an embodiment herein. Particularly the fragment of a polynucleotide comprises at least 25, more particularly at least 50, more particularly at least 75, more particularly at least 100, more particularly at least 150, more particularly at least 200, more particularly at least 300, more particularly at least 400, more particularly at least 500, more particularly at least 600, more particularly at least 700, more particularly at least 800, more particularly at least 900, more particularly at least 1000 contiguous nucleotides of the polynucleotide of an embodiment herein. Without being limited, the fragment of the polynucleotides herein may be used as a PCR primer, and/or as a probe, or for anti-sense gene silencing or RNAi.

As used herein, the term “hybridization” or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein below. Appropriate hybridization conditions can also be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

“Recombinant nucleic acid sequences” are nucleic acid sequences that result from the use of laboratory methods (for example, molecular cloning) to bring together genetic material from more than on source, creating or modifying a nucleic acid sequence that does not occur naturally and would not be otherwise found in biological organisms.

“Recombinant DNA technology” refers to molecular biology procedures to prepare a recombinant nucleic acid sequence as described, for instance, in Laboratory Manuals edited by Weigel and Glazebrook, 2002, Cold Spring Harbor Lab Press; and Sambrook et al., 1989, Cold Spring Harbor, NY, Cold Spring Harbor Laboratory Press.

The term “gene” means a DNA sequence comprising a region, which is transcribed into a RNA molecule, e.g., an mRNA in a cell, operably linked to suitable regulatory regions, e.g., a promoter. A gene may thus comprise several operably linked sequences, such as a promoter, a 5′ leader sequence comprising. e.g., sequences involved in translation initiation, a coding region of cDNA or genomic DNA, introns, exons, and/or a 3′non-translated sequence comprising, e.g., transcription termination sites.

“Polycistronic” refers to nucleic acid molecules, in particular mRNAs, that can encode more than one polypeptide separately within the same nucleic acid molecule

A “chimeric gene” refers to any gene which is not normally found in nature in a species, in particular, a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense, i.e., reverse complement of the sense strand, or inverted repeat sequence (sense and antisense, whereby the RNA transcript forms double stranded RNA upon transcription). The term “chimeric gene” also includes genes obtained through the combination of portions of one or more coding sequences to produce a new gene.

A “3′ UTR” or “3′ non-translated sequence” (also referred to as “3′ untranslated region,” or “3′end”) refers to the nucleic acid sequence found downstream of the coding sequence of a gene, which comprises, for example, a transcription termination site and (in most, but not all eukaryotic mRNAs) a polyadenylation signal such as AAUAAA or variants thereof. After termination of transcription, the mRNA transcript may be cleaved downstream of the polyadenylation signal and a poly(A) tail may be added, which is involved in the transport of the mRNA to the site of translation, e.g., cytoplasm.

The term “primer” refers to a short nucleic acid sequence that is hybridized to a template nucleic acid sequence and is used for polymerization of a nucleic acid sequence complementary to the template.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

The invention also relates to nucleic acid sequences that code for polypeptides as defined herein.

In particular, the invention also relates to nucleic acid sequences (single-stranded and double-stranded DNA and RNA sequences, e.g. cDNA, genomic DNA and mRNA), coding for one of the above polypeptides and their functional equivalents, which can be obtained for example using artificial nucleotide analogs.

The invention relates both to isolated nucleic acid molecules, which code for polypeptides according to the invention or biologically active segments thereof, and to nucleic acid fragments, which can be used for example as hybridization probes or primers for identifying or amplifying coding nucleic acids according to the invention.

The present invention also relates to nucleic acids with a certain degree of “identity” to the sequences specifically disclosed herein. “Identity” between two nucleic acids means identity of the nucleotides, in each case over the entire length of the nucleic acid.

The “identity.” between two nucleotide sequences (the same applies to peptide or amino acid sequences) is a function of the number of nucleotide residues (or amino acid residues) or that are identical in the two sequences when an alignment of these two sequences has been generated. Identical residues are defined as residues that are the same in the two sequences in a given position of the alignment. The percentage of sequence identity, as used herein, is calculated from the optimal alignment by taking the number of residues identical between two sequences dividing it by the total number of residues in the shortest sequence and multiplying by 100. The optimal alignment is the alignment in which the percentage of identity is the highest possible. Gaps may be introduced into one or both sequences in one or more positions of the alignment to obtain the optimal alignment. These gaps are then taken into account as non-identical residues for the calculation of the percentage of sequence identity. Alignment for the purpose of determining the percentage of amino acid or nucleic acid sequence identity can be achieved in various ways using computer programs and for instance publicly available computer programs available on the world wide web.

Particularly, the BLAST program (Tatiana et al, FEMS Microbiol Lett., 1999, 174:247-250, 1999) set to the default parameters, available from the National Center for Biotechnology Information (NCBI) website at ncbi.nlm.nih.gov/BLAST/bl2seq/wblast2.cgi, can be used to obtain an optimal alignment of protein or nucleic acid sequences and to calculate the percentage of sequence identity.

In another example the identity may be calculated by means of the Vector NTI Suite 7.1 program of the company Informax (USA) employing the Clustal Method (Higgins D G, Sharp P M. ((1989))) with the following settings:

| Multiple alignment parameters: | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |
| Pairwise alignment parameter: | |
| FAST algorithm | on |
| K-tuple size | 1 |
| Gap penalty | 3 |
| Window size | 5 |
| Number of best diagonals | 5 |

Alternatively the identity may be determined according to Chenna, et al. (2003), the web page: www.ebi.ac.uk/Tools/clustalw/index.html # and the following settings

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | −1 |
| Protein/DNA GAPDIST | 4 |

All the nucleic acid sequences mentioned herein (single-stranded and double-stranded DNA and RNA sequences, for example cDNA and mRNA) can be produced in a known way by chemical synthesis from the nucleotide building blocks, e.g. by fragment condensation of individual overlapping, complementary nucleic acid building blocks of the double helix. Chemical synthesis of oligonucleotides can, for example, be performed in a known way, by the phosphoamidite method (Voet, Voet, 2nd edition, Wiley Press, New York, pages 896-897). The accumulation of synthetic oligonucleotides and filling of gaps by means of the Klenow fragment of DNA polymerase and ligation reactions as well as general cloning techniques are described in Sambrook et al. (1989), see below.

The nucleic acid molecules according to the invention can in addition contain non-translated sequences from the 3′ and/or 5′ end of the coding genetic region.

The invention further relates to the nucleic acid molecules that are complementary to the concretely described nucleotide sequences or a segment thereof.

The nucleotide sequences according to the invention make possible the production of probes and primers that can be used for the identification and/or cloning of homologous sequences in other cellular types and organisms. Such probes or primers generally comprise a nucleotide sequence region which hybridizes under “stringent” conditions (as defined herein elsewhere) on at least about 12, preferably at least about 25, for example about 40, 50 or 75 successive nucleotides of a sense strand of a nucleic acid sequence according to the invention or of a corresponding antisense strand.

“Homologous” sequences include orthologous or paralogous sequences. Methods of identifying orthologs or paralogs including phylogenetic methods, sequence similarity and hybridization methods are known in the art and are described herein.

“Paralogs” result from gene duplication that gives rise to two or more genes with similar sequences and similar functions. Paralogs typically cluster together and are formed by duplications of genes within related plant species. Paralogs are found in groups of similar genes using pair-wise Blast analysis or during phylogenetic analysis of gene families using programs such as CLUSTAL. In paralogs, consensus sequences can be identified characteristic to sequences within related genes and having similar functions of the genes.

“Orthologs”, or orthologous sequences, are sequences similar to each other because they are found in species that descended from a common ancestor. For instance, plant species that have common ancestors are known to contain many enzymes that have similar sequences and functions. The skilled artisan can identify orthologous sequences and predict the functions of the orthologs, for example, by constructing a polygenic tree for a gene family of one species using CLUSTAL or BLAST programs. A method for identifying or confirming similar functions among homologous sequences is by comparing of the transcript profiles in host cells or organisms, such as plants or microorganisms, overexpressing or lacking (in knockouts/knockdowns) related polypeptides. The skilled person will understand that genes having similar transcript profiles, with greater than 50% regulated transcripts in common, or with greater than 70% regulated transcripts in common, or greater than 90% regulated transcripts in common will have similar functions. Homologs, paralogs, orthologs and any other variants of the sequences herein are expected to function in a similar manner by making the host cells, organism such as plants or microorganisms producing terpene synthase proteins.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

A nucleic acid molecule according to the invention can be recovered by means of standard techniques of molecular biology and the sequence information supplied according to the invention. For example, cDNA can be isolated from a suitable cDNA library, using one of the concretely disclosed complete sequences or a segment thereof as hybridization probe and standard hybridization techniques (as described for example in Sambrook, (1989)).

In addition, a nucleic acid molecule comprising one of the disclosed sequences or a segment thereof, can be isolated by the polymerase chain reaction, using the oligonucleotide primers that were constructed on the basis of this sequence. The nucleic acid amplified in this way can be cloned in a suitable vector and can be characterized by DNA sequencing. The oligonucleotides according to the invention can also be produced by standard methods of synthesis, e.g. using an automatic DNA synthesizer.

Nucleic acid sequences according to the invention or derivatives thereof, homologues or parts of these sequences, can for example be isolated by usual hybridization techniques or the PCR technique from other bacteria, e.g. via genomic or cDNA libraries. These DNA sequences hybridize in standard conditions with the sequences ac-cording to the invention.

“Hybridize” means the ability of a polynucleotide or oligonucleotide to bind to an almost complementary sequence in standard conditions, whereas nonspecific binding does not occur between non-complementary partners in these conditions. For this, the sequences can be 90-100% complementary. The property of complementary sequences of being able to bind specifically to one another is utilized for example in Northern Blotting or Southern Blotting or in primer binding in PCR or RT-PCR.

Short oligonucleotides of the conserved regions are used advantageously for hybridization. However, it is also possible to use longer fragments of the nucleic acids according to the invention or the complete sequences for the hybridization. These “standard conditions” vary depending on the nucleic acid used (oligonucleotide, longer fragment or complete sequence) or depending on which type of nucleic acid—DNA or RNA—is used for hybridization. For example, the melting temperatures for DNA:DNA hybrids are approx. 10° C. lower than those of DNA:RNA hybrids of the same length.

For example, depending on the particular nucleic acid, standard conditions mean temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC, 50% formamide. Advantageously, the hybridization conditions for DNA:DNA hybrids are 0.1×SSC and temperatures between about 20° C., to 45° C., preferably between about 30° C., to 45° C. For DNA:RNA hybrids the hybridization conditions are advantageously 0.1×SSC and temperatures between about 30° C. to 55° C., preferably between about 45° C., to 55° C. These stated temperatures for hybridization are examples of calculated melting temperature values for a nucleic acid with a length of approx. 100 nucleotides and a G+C content of 50% in the absence of formamide. The experimental conditions for DNA hybridization are described in relevant genetics textbooks, for example Sambrook et al., 1989, and can be calculated using formulae that are known by a person skilled in the art, for example depending on the length of the nucleic acids, the type of hybrids or the G+C content. A person skilled in the art can obtain further information on hybridization from the following textbooks: Ausubel et al. (eds), (1985), Brown (ed) (1991).

“Hybridization” can in particular be carried out under stringent conditions. Such hybridization conditions are for example described in Sambrook (1989), or in Current Protocols in Molecular Biology, John Wiley & Sons, N.Y. (1989), 6.3.1-6.3.6.

As used herein, the term hybridization or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein.

Appropriate hybridization conditions can be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

As used herein, defined conditions of low stringency are as follows. Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP. 0.02% Ficoll, 0.2% BSA, 100 μg/ml salmon sperm DNA, 10% (wt/vol) dextran sulfate, and 5-20×106 32P-labeled probe is used. Filters are incubated in hybridization mixture for 18-20 h at 40° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of moderate stringency are as follows. Filters containing DNA are pretreated for 7 h at 50° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×106 32P-labeled probe is used. Filters are incubated in hybridization mixture for 30 h at 50° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in the prehybridization mixture containing 100 g/ml denatured salmon sperm DNA and 5-20×106 cpm of 32P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes.

Other conditions of low, moderate, and high stringency well known in the art (e.g., as employed for cross-species hybridizations) may be used if the above conditions are inappropriate (e.g., as employed for cross-species hybridizations).

A detection kit for nucleic acid sequences encoding a polypeptide of the invention may include primers and/or probes specific for nucleic acid sequences encoding the polypeptide, and an associated protocol to use the primers and/or probes to detect nucleic acid sequences encoding the polypeptide in a sample. Such detection kits may be used to determine whether a plant, organism, microorganism or cell has been modified, i.e., transformed with a sequence encoding the polypeptide.

To test a function of variant DNA sequences according to an embodiment herein, the sequence of interest is operably linked to a selectable or screenable marker gene and expression of said reporter gene is tested in transient expression assays, for example, with microorganisms or with protoplasts or in stably transformed plants.

The invention also relates to derivatives of the concretely disclosed or derivable nucleic acid sequences.

Thus, further nucleic acid sequences according to the invention can be derived from the sequences specifically disclosed herein and can differ from it by one or more, like 1 to 20, in particular 1 to 15 or 5 to 10 additions, substitutions, insertions or deletions of one or several (like for example 1 to 10) nucleotides, and furthermore code for polypeptides with the desired profile of properties.

The invention also encompasses nucleic acid sequences that comprise so-called silent mutations or have been altered, in comparison with a concretely stated sequence, according to the codon usage of a special original or host organism.

According to a particular embodiment of the invention variant nucleic acids may be prepared in order to adapt its nucleotide sequence to a specific expression system. For example, bacterial expression systems are known to more efficiently express polypeptides if amino acids are encoded by particular codons. Due to the degeneracy of the genetic code, more than one codon may encode the same amino acid sequence, multiple nucleic acid sequences can code for the same protein or polypeptide, all these DNA sequences being encompassed by an embodiment herein. Where appropriate, the nucleic acid sequences encoding the polypeptides described herein may be optimized for increased expression in the host cell. For example, nucleic acids of an embodiment herein may be synthesized using codons particular to a host for improved expression.

The invention also encompasses naturally occurring variants, e.g. splicing variants or allelic variants, of the sequences described therein.

Allelic variants may have at least 60% homology at the level of the derived amino acid, preferably at least 80% homology, quite especially preferably at least 90% homology over the entire sequence range (regarding homology at the amino acid level, reference should be made to the details given above for the polypeptides). Advantageously, the homologies can be higher over partial regions of the sequences.

The invention also relates to sequences that can be obtained by conservative nucleotide substitutions (i.e, as a result thereof the amino acid in question is replaced by an amino acid of the same charge, size, polarity and/or solubility).

The invention also relates to the molecules derived from the concretely disclosed nucleic acids by sequence polymorphisms. Such genetic polymorphisms may exist in cells from different populations or within a population due to natural allelic variation. Allelic variants may also include functional equivalents. These natural variations usually produce a variance of 1 to 5% in the nucleotide sequence of a gene. Said polymorphisms may lead to changes in the amino acid sequence of the polypeptides disclosed herein. Allelic variants may also include functional equivalents.

Furthermore, derivatives are also to be understood to be homologs of the nucleic acid sequences according to the invention, for example animal, plant, fungal or bacterial homologs, shortened sequences, single-stranded DNA or RNA of the coding and noncoding DNA sequence. For example, homologs have, at the DNA level, a homology of at least 40%, preferably of at least 60%, especially preferably of at least 70%, quite especially preferably of at least 80% over the entire DNA region given in a sequence specifically disclosed herein.

Moreover, derivatives are to be understood to be, for example, fusions with promoters. The promoters that are added to the stated nucleotide sequences can be modified by at least one nucleotide exchange, at least one insertion, inversion and/or deletion, though without impairing the functionality or efficacy of the promoters. Moreover, the efficacy of the promoters can be increased by altering their sequence or can be exchanged completely with more effective promoters even of organisms of a different genus.

### d. Generation of Functional Polypeptide Mutants

Moreover, a person skilled in the art is familiar with methods for generating functional mutants, that is to say nucleotide sequences which code for a polypeptide with at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity to anyone of amino acid related SEQ ID NOs as disclosed herein and/or encoded by a nucleic acid molecule comprising a nucleotide sequence having at least 70% sequence identity to anyone of the nucleotide related SEQ ID NOs as disclosed herein.

Depending on the technique used, a person skilled in the art can introduce entirely random or else more directed mutations into genes or else noncoding nucleic acid regions (which are for example important for regulating expression) and subsequently generate genetic libraries. The methods of molecular biology required for this purpose are known to the skilled worker and for example described in Sambrook and Russell, Molecular Cloning. 3rd Edition, Cold Spring Harbor Laboratory Press 2001.

Methods for modifying genes and thus for modifying the polypeptide encoded by them have been known to the skilled worker for a long time, such as, for example
site-specific mutagenesis, where individual or several nucleotides of a gene are replaced in a directed fashion (Trower MK (Ed.) 1996; In vitro mutagenesis protocols. Humana Press, New Jersey),
saturation mutagenesis, in which a codon for any amino acid can be exchanged or added at any point of a gene (Kegler-Ebo D M, Docktor C M, DiMaio D (1994) Nucleic Acids Res 22:1593; Barettino D, Feigenbutz M, Valcarel R, Stunnenberg HG (1994) Nucleic Acids Res 22:541; Barik S (1995) Mol Biotechnol 3:1),
error-prone polymerase chain reaction, where nucleotide sequences are mutated by error-prone DNA polymerases (Eckert K A, Kunkel T A (1990) Nucleic Acids Res 18:3739);
the SeSaM method (sequence saturation method), in which preferred exchanges are prevented by the polymerase. Schenk et al., Biospektrum, Vol. 3, 2006, 277-279
the passaging of genes in mutator strains, in which, for example owing to defective DNA repair mechanisms, there is an increased mutation rate of nucleotide sequences (Greener A, Callahan M, Jerpseth B (1996) An efficient random mutagenesis technique using an E. coli mutator strain. In: Trower MK (Ed.) In vitro mutagenesis protocols. Humana Press, New Jersey), or
DNA shuffling, in which a pool of closely related genes is formed and digested and the fragments are used as templates for a polymerase chain reaction in which, by repeated strand separation and reassociation, full-length mosaic genes are ultimately generated (Stemmer WPC (1994) Nature 370:389; Stemmer WPC (1994) Proc Natl Acad Sci USA 91:10747).

Using so-called directed evolution (described, inter alia, in Reetz MT and Jaeger K-E (1999), Topics Curr Chem 200:31; Zhao H, Moore J C, Volkov A A, Arnold F H (1999), Methods for optimizing industrial polypeptides by directed evolution, In: Demain A L, Davies J E (Ed.) Manual of industrial microbiology and biotechnology. American Society for Microbiology), a skilled worker can produce functional mutants in a directed manner and on a large scale. To this end, in a first step, gene libraries of the respective polypeptides are first produced, for example using the methods given above. The gene libraries are expressed in a suitable way, for example by bacteria or by phage display systems.

The relevant genes of host organisms which express functional mutants with properties that largely correspond to the desired properties can be submitted to another mutation cycle. The steps of the mutation and selection or screening can be repeated iteratively until the present functional mutants have the desired properties to a sufficient extent. Using this iterative procedure, a limited number of mutations, for example 1, 2, 3, 4 or 5 mutations, can be performed in stages and assessed and selected for their influence on the activity in question. The selected mutant can then be submitted to a further mutation step in the same way. In this way, the number of individual mutants to be investigated can be reduced significantly.

The results according to the invention also provide important information relating to structure and sequence of the relevant polypeptides, which is required for generating, in a targeted fashion, further polypeptides with desired modified properties. In particular, it is possible to define so-called “hot spots”, i.e. sequence segments that are potentially suitable for modifying a property by introducing targeted mutations.

Information can also be deduced regarding amino acid sequence positions, in the region of which mutations can be effected that should probably have little effect on the activity, and can be designated as potential “silent mutations”.

### e. Constructs for Expressing Polypeptides of the Invention

In this context the following definitions apply:

“Expression of a gene” encompasses “heterologous expression” and “over-expression” and involves transcription of the gene and translation of the mRNA into a protein. Overexpression refers to the production of the gene product as measured by levels of mRNA, polypeptide and/or enzyme activity in transgenic cells or organisms that exceeds levels of production in non-transformed cells or organisms of a similar genetic background.

“Expression vector” as used herein means a nucleic acid molecule engineered using molecular biology methods and recombinant DNA technology for delivery of foreign or exogenous DNA into a host cell. The expression vector typically includes sequences required for proper transcription of the nucleotide sequence. The coding region usually codes for a protein of interest but may also code for an RNA, e.g., an antisense RNA, siRNA and the like.

An “expression vector” as used herein includes any linear or circular recombinant vector including but not limited to viral vectors, bacteriophages and plasmids. The skilled person is capable of selecting a suitable vector according to the expression system. In one embodiment, the expression vector includes the nucleic acid of an embodiment herein operably linked to at least one “regulatory sequence”, which controls transcription, translation, initiation and termination, such as a transcriptional promoter, operator or enhancer, or an mRNA ribosomal binding site and, optionally, including at least one selection marker. Nucleotide sequences are “operably linked” when the regulatory sequence functionally relates to the nucleic acid of an embodiment herein.

An “expression system” as used herein encompasses any combination of nucleic acid molecules required for the expression of one, or the co-expression of two or more polypeptides either in vivo of a given expression host, or in vitro. The respective coding sequences may either be located on a single nucleic acid molecule or vector, as for example a vector containing multiple cloning sites, or on a polycistronic nucleic acid, or may be distributed over two or more physically distinct vectors. As a particular example there may be mentioned an operon comprising a promotor sequence, one or more operator sequences and one or more structural genes each encoding an enzyme as described herein

As used herein, the terms “amplifying” and “amplification” refer to the use of any suitable amplification methodology for generating or detecting recombinant of naturally expressed nucleic acid, as described in detail, below. For example, the invention provides methods and reagents (e.g., specific degenerate oligonucleotide primer pairs, oligo dT primer) for amplifying (e.g., by polymerase chain reaction, PCR) naturally expressed (e.g., genomic DNA or mRNA) or recombinant (e.g., cDNA) nucleic acids of the invention in vivo, ex vivo or in vitro.

“Regulatory sequence” refers to a nucleic acid sequence that determines expression level of the nucleic acid sequences of an embodiment herein and is capable of regulating the rate of transcription of the nucleic acid sequence operably linked to the regulatory sequence. Regulatory sequences comprise promoters, enhancers, transcription factors, promoter elements and the like.

A “promoter”, a “nucleic acid with promoter activity” or a “promoter sequence” is understood as meaning, in accordance with the invention, a nucleic acid which, when functionally linked to a nucleic acid to be transcribed, regulates the transcription of said nucleic acid. “Promoter” in particular refers to a nucleic acid sequence that controls the expression of a coding sequence by providing a binding site for RNA polymerase and other factors required for proper transcription including without limitation transcription factor binding sites, repressor and activator protein binding sites. The meaning of the term promoter also includes the term “promoter regulatory sequence”. Promoter regulatory sequences may include upstream and downstream elements that may influences transcription, RNA processing or stability of the associated coding nucleic acid sequence. Promoters include naturally-derived and synthetic sequences. The coding nucleic acid sequences is usually located downstream of the promoter with respect to the direction of the transcription starting at the transcription initiation site.

In this context, a “functional” or “operative” linkage is understood as meaning for example the sequential arrangement of one of the nucleic acids with a regulatory sequence. For example the sequence with promoter activity and of a nucleic acid sequence to be transcribed and optionally further regulatory elements, for example nucleic acid sequences which ensure the transcription of nucleic acids, and for example a terminator, are linked in such a way that each of the regulatory elements can perform its function upon transcription of the nucleic acid sequence. This does not necessarily require a direct linkage in the chemical sense. Genetic control sequences, for example enhancer sequences, can even exert their function on the target sequence from more remote positions or even from other DNA molecules. Preferred arrangements are those in which the nucleic acid sequence to be transcribed is positioned behind (i.e, at the 3′-end of) the promoter sequence so that the two sequences are joined together covalently. The distance between the promoter sequence and the nucleic acid sequence to be expressed recombinantly can be smaller than 200 base pairs, or smaller than 100 base pairs or smaller than 50 base pairs.

In addition to promoters and terminator, the following may be mentioned as examples of other regulatory elements: targeting sequences, enhancers, polyadenylation signals, selectable markers, amplification signals, replication origins and the like. Suitable regulatory sequences are described, for example, in Goeddel. Gene Expression Technology: Methods in Enzymology 185. Academic Press. San Diego. CA (1990).

The term “constitutive promoter” refers to an unregulated promoter that allows for continual transcription of the nucleic acid sequence it is operably linked to.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous. The nucleotide sequence associated with the promoter sequence may be of homologous or heterologous origin with respect to the plant to be transformed. The sequence also may be entirely or partially synthetic. Regardless of the origin, the nucleic acid sequence associated with the promoter sequence will be expressed or silenced in accordance with promoter properties to which it is linked after binding to the polypeptide of an embodiment herein. The associated nucleic acid may code for a protein that is desired to be expressed or suppressed throughout the organism at all times or, alternatively, at a specific time or in specific tissues, cells, or cell compartment. Such nucleotide sequences particularly encode proteins conferring desirable phenotypic traits to the host cells or organism altered or transformed therewith. More particularly, the associated nucleotide sequence leads to the production of the product or products of interest as herein defined in the cell or organism. Particularly, the nucleotide sequence encodes a polypeptide having an enzyme activity as herein defined.

The nucleotide sequence as described herein above may be part of an “expression cassette”. The terms “expression cassette” and “expression construct” are used synonymously. The (preferably recombinant) expression construct contains a nucleotide sequence which encodes a polypeptide according to the invention and which is under genetic control of regulatory nucleic acid sequences.

In a process applied according to the invention, the expression cassette may be part of an “expression vector”, in particular of a recombinant expression vector.

An “expression unit” is understood as meaning, in accordance with the invention, a nucleic acid with expression activity which comprises a promoter as defined herein and, after functional linkage with a nucleic acid to be expressed or a gene, regulates the expression, i.e. the transcription and the translation of said nucleic acid or said gene. It is therefore in this connection also referred to as a “regulatory nucleic acid sequence”. In addition to the promoter, other regulatory elements, for example enhancers, can also be present.

An “expression cassette” or “expression construct” is understood as meaning, in accordance with the invention, an expression unit which is functionally linked to the nucleic acid to be expressed or the gene to be expressed. In contrast to an expression unit, an expression cassette therefore comprises not only nucleic acid sequences which regulate transcription and translation, but also the nucleic acid sequences that are to be expressed as protein as a result of transcription and translation.

The terms “expression” or “overexpression” describe, in the context of the invention, the production or increase in intracellular activity of one or more polypeptides in a microorganism, which are encoded by the corresponding DNA. To this end, it is possible for example to introduce a gene into an organism, replace an existing gene with another gene, increase the copy number of the gene(s), use a strong promoter or use a gene which encodes for a corresponding polypeptide with a high activity: optionally, these measures can be combined.

Preferably such constructs according to the invention comprise a promoter 5′-upstream of the respective coding sequence and a terminator sequence 3′-downstream and optionally other usual regulatory elements, in each case in operative linkage with the coding sequence.

Nucleic acid constructs according to the invention comprise in particular a sequence coding for a polypeptide for example derived from the amino acid related SEQ ID NOs as described therein or the reverse complement thereof, or derivatives and homologs thereof and which have been linked operatively or functionally with one or more regulatory signals, advantageously for controlling, for example increasing, gene expression.

In addition to these regulatory sequences, the natural regulation of these sequences may still be present before the actual structural genes and optionally may have been genetically modified so that the natural regulation has been switched off and expression of the genes has been enhanced. The nucleic acid construct may, however, also be of simpler construction, i.e. no additional regulatory signals have been inserted before the coding sequence and the natural promoter, with its regulation, has not been removed. Instead, the natural regulatory sequence is mutated such that regulation no longer takes place and the gene expression is increased.

A preferred nucleic acid construct advantageously also comprises one or more of the already mentioned “enhancer” sequences in functional linkage with the promoter, which sequences make possible an enhanced expression of the nucleic acid sequence. Additional advantageous sequences may also be inserted at the 3′-end of the DNA sequences, such as further regulatory elements or terminators. One or more copies of the nucleic acids according to the invention may be present in a construct. In the construct, other markers, such as genes which complement auxotrophisms or antibiotic resistances, may also optionally be present so as to select for the construct.

Examples of suitable regulatory sequences are present in promoters such as cos, tac, trp, tet, trp-tet, lpp, lac, lpp-lac, lac1^{q}, T7, T5, T3, gal, trc, ara, rhaP (rhaP_{BAD})SP6, lambda-P_{R} or in the lambda-P_{L} promoter, and these are advantageously employed in Gram-negative bacteria. Further advantageous regulatory sequences are present for example in the Gram-positive promoters amy and SPO2, in the yeast or fungal promoters ADC1, MFalpha, AC, P-60, CYC1, GAPDH, TEF, rp28, ADH. Artificial promoters may also be used for regulation.

For expression in a host organism, the nucleic acid construct is inserted advantageously into a vector such as, for example, a plasmid or a phage, which makes possible optimal expression of the genes in the host. Vectors are also understood as meaning, in addition to plasmids and phages, all the other vectors which are known to the skilled worker, that is to say for example viruses such as SV40, CMV, baculovirus and adenovirus, transposons, IS elements, phasmids, cosmids and linear or circular DNA or artificial chromosomes. These vectors are capable of replicating autonomously in the host organism or else chromosomally. These vectors are a further development of the invention. Binary or cpo-integration vectors are also applicable.

Suitable plasmids are, for example, in E. coli pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCI, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alphaM, pAG-1, YEp6, YEp13 or pEMBLYe23 or in plants pLGV23, pGHlac⁺, pBIN19, pAK2004 or pDH51. The abovementioned plasmids are a small selection of the plasmids which are possible. Further plasmids are well known to the skilled worker and can be found for example in the book Cloning Vectors (Eds. Pouwels P. H. et al. Elsevier, Amsterdam-New York-Oxford, 1985, ISBN 0 444 904018).

In a further development of the vector, the vector which comprises the nucleic acid construct according to the invention or the nucleic acid according to the invention can advantageously also be introduced into the microorganisms in the form of a linear DNA and integrated into the host organism's genome via heterologous or homologous recombination. This linear DNA can consist of a linearized vector such as a plasmid or only of the nucleic acid construct or the nucleic acid according to the invention.

For optimal expression of heterologous genes in organisms, it is advantageous to modify the nucleic acid sequences to match the specific “codon usage” used in the organism. The “codon usage” can be determined readily by computer evaluations of other, known genes of the organism in question.

An expression cassette according to the invention is generated by fusing a suitable promoter to a suitable coding nucleotide sequence and a terminator or polyadenylation signal. Customary recombination and cloning techniques are used for this purpose, as are described, for example, in T. Maniatis, E. F. Fritsch and J. Sambrook, Molecular Cloning: A Laboratory Manual, Cold Spring Harbor Laboratory, Cold Spring Harbor, NY (1989) and in T. J. Silhavy. M. L. Berman and L. W. Enquist, Experiments with Gene Fusions, Cold Spring Harbor Laboratory, Cold Spring Harbor, NY (1984) and in Ausubel, F. M. et al., Current Protocols in Molecular Biology, Greene Publishing Assoc, and Wiley Interscience (1987).

For expression in a suitable host organism, the recombinant nucleic acid construct or gene construct is advantageously inserted into a host-specific vector which makes possible optimal expression of the genes in the host. Vectors are well known to the skilled worker and can be found for example in “cloning vectors” (Pouwels P. H. et al., Ed., Elsevier, Amsterdam-New York-Oxford, 1985).

An alternative embodiment of an embodiment herein provides a method to “alter gene expression” in a host cell. For instance, the polynucleotide of an embodiment herein may be enhanced or overexpressed or induced in certain contexts (e.g. upon exposure to certain temperatures or culture conditions) in a host cell or host organism.

Alteration of expression of a polynucleotide provided herein may also result in ectopic expression which is a different expression pattern in an altered and in a control or wild-type organism. Alteration of expression occurs from interactions of polypeptide of an embodiment herein with exogenous or endogenous modulators, or as a result of chemical modification of the polypeptide. The term also refers to an altered expression pattern of the polynucleotide of an embodiment herein which is altered below the detection level or completely suppressed activity.

In one embodiment, provided herein is also an isolated, recombinant or synthetic polynucleotide encoding a polypeptide or variant polypeptide provided herein.

In one embodiment, several polypeptide encoding nucleic acid sequences are co-expressed in a single host, particularly under control of different promoters. In another embodiment, several polypeptide encoding nucleic acid sequences can be present on a single transformation vector or be co-transformed at the same time using separate vectors and selecting transformants comprising both chimeric genes. Similarly, one or polypeptide encoding genes may be expressed in a single plant, cell, microorganism or organism together with other chimeric genes.

### f. Hosts to be Applied for the Present Invention

Depending on the context, the term “host” can mean the wild-type host or a genetically altered, recombinant host or both.

In principle, all prokaryotic or eukaryotic organisms may be considered as host or recombinant host organisms for the nucleic acids or the nucleic acid constructs according to the invention.

Using the vectors according to the invention, recombinant hosts can be produced, which are for example transformed with at least one vector according to the invention and can be used for producing the polypeptides according to the invention. Advantageously, the recombinant constructs according to the invention, described above, are introduced into a suitable host system and expressed. Preferably common cloning and transfection methods, known by a person skilled in the art, are used, for example co-precipitation, protoplast fusion, electroporation, retroviral transfection and the like, for expressing the stated nucleic acids in the respective expression system. Suitable systems are described for example in Current Protocols in Molecular Biology, F. Ausubel et al., Ed., Wiley Interscience, New York 1997, or Sambrook et al. Molecular Cloning: A Laboratory Manual. 2nd edition, Cold Spring Harbor Laboratory, Cold Spring Harbor Laboratory Press, Cold Spring Harbor, NY, 1989.

Advantageously, microorganisms such as bacteria, fungi or yeasts are used as host organisms. Advantageously, gram-positive or gram-negative bacteria are used, preferably bacteria of the families Enterobacteriaceae. Pseudomonadaceae. Rhizobiaceae. Streptomycetaceae, Streptococcaceae or Nocardiaceae, especially preferably bacteria of the genera Escherichia, Pseudomonas, Streptomyces, Lactococcus, Nocardia, Burkholderia, Salmonella, Agrobacterium, Clostridium or Rhodococcus. The genus and species Escherichia coli is quite especially preferred. Furthermore, other advantageous bacteria are to be found in the group of alpha-Proteobacteria, beta-Proteobacteria or gamma-Proteobacteria. Advantageously also yeasts of families like Saccharomyces or Pichia are suitable hosts.

Alternatively, entire plants or plant cells may serve as natural or recombinant host. As non-limiting examples the following plants or cells derived therefrom may be mentioned the genera Nicotiana, in particular Nicotiana benthamiana and Nicotiana tabacum (tobacco); as well as Arabidopsis, in particular Arabidopsis thaliana.

Depending on the host organism, the organisms used in the method according to the invention are grown or cultured in a manner known by a person skilled in the art. Culture can be batchwise, semi-batchwise or continuous. Nutrients can be present at the beginning of fermentation or can be supplied later, semicontinuously or continuously. This is also described in more detail below.

### g. Recombinant Production of Polypeptides According to the Invention

The invention further relates to methods for recombinant production of polypeptides according to the invention or functional, biologically active fragments thereof, wherein a polypeptide-producing microorganism is cultured, optionally the expression of the polypeptides is induced by applying at least one inducer inducing gene expression and the expressed polypeptides are isolated from the culture. The polypeptides can also be produced in this way on an industrial scale, if desired.

The microorganisms produced according to the invention can be cultured continuously or discontinuously in the batch method or in the fed-batch method or repeated fed-batch method. A summary of known cultivation methods can be found in the textbook by Chmiel (Bioprozesstechnik l. Einführung in die Bioverfahrenstechnik [Bioprocess technology 1. Introduction to bioprocess technology] (Gustav Fischer Verlag, Stuttgart, 1991)) or in the textbook by Storhas (Bioreaktoren und periphere Einrichtungen [Bioreactors and peripheral equipment] (Vieweg Verlag, Braunschweig/Wiesbaden, 1994)).

The culture medium to be used must suitably meet the requirements of the respective strains. Descriptions of culture media for various microorganisms are given in the manual “Manual of Methods for General Bacteriology” of the American Society for Bacteriology (Washington D. C., USA, 1981).

These media usable according to the invention usually comprise one or more carbon sources, nitrogen sources, inorganic salts, vitamins and/or trace elements.

Preferred carbon sources are sugars, such as mono-, di- or polysaccharides. Very good carbon sources are for example glucose, fructose, mannose, galactose, ribose, sorbose, ribulose, lactose, maltose, sucrose, raffinose, starch or cellulose. Sugars can also be added to the media via complex compounds, such as molasses, or other by-products of sugar refining. It can also be advantageous to add mixtures of different carbon sources. Other possible carbon sources are oils and fats, for example soy bean oil, sunflower oil, peanut oil and coconut oil, fatty acids, for example palmitic acid, stearic acid or linoleic acid, alcohols, for example glycerol, methanol or ethanol and organic acids, for example acetic acid or lactic acid.

Nitrogen sources are usually organic or inorganic nitrogen compounds or materials that contain these compounds. Examples of nitrogen sources comprise ammonia gas or ammonium salts, such as ammonium sulfate, ammonium chloride, ammonium phosphate, ammonium carbonate or ammonium nitrate, nitrates, urea, amino acids or complex nitrogen sources, such as corn-steep liquor, soya flour, soya protein, yeast extract, meat extract and others. The nitrogen sources can be used alone or as a mixture.

Inorganic salt compounds that can be present in the media comprise the chloride, phosphorus or sulfate salts of calcium, magnesium, sodium, cobalt, molybdenum, potassium, manganese, zinc, copper and iron.

Inorganic sulfur-containing compounds, for example sulfates, sulfites, dithionites, tetrathionates, thiosulfates, sulfides, as well as organic sulfur compounds, such as mercaptans and thiols, can be used as the sulfur source.

Phosphoric acid, potassium dihydrogen phosphate or dipotassium hydrogen phosphate or the corresponding sodium-containing salts can be used as the phosphorus source.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention usually also contain other growth factors, such as vitamins or growth promoters, which include for example biotin, riboflavin, thiamine, folic acid, nicotinic acid, pantothenate and pyridoxine. Growth factors and salts often originate from the components of complex media, such as yeast extract, molasses, corn-steep liquor and the like. Moreover, suitable precursors can be added to the culture medium. The exact composition of the compounds in the medium is strongly dependent on the respective experiment and is decided for each specific case individually. Information on media optimization can be found in the textbook “Applied Microbiol. Physiology, A Practical Approach” (Ed. P. M. Rhodes, P. F. Stanbury, IRL Press (1997) p. 53-73, ISBN 0 19 963577 3). Growth media can also be obtained from commercial suppliers, such as Standard 1 (Merck) or BHI (brain heart infusion, DIFCO) and the like.

All components of the medium are sterilized, either by heat (20 min at 1.5 bar and 121° C.) or by sterile filtration. The components can either be sterilized together, or separately if necessary. All components of the medium can be present at the start of culture or can be added either continuously or batchwise.

The culture temperature is normally between 15° C., and 45° C., preferably 25° C., to 40° C. and can be varied or kept constant during the experiment. The pH of the medium should be in the range from 5 to 8.5, preferably around 7.0. The pH for growing can be controlled during growing by adding basic compounds such as sodium hydroxide, potassium hydroxide, ammonia or ammonia water or acid compounds such as phosphoric acid or sulfuric acid. Antifoaming agents, for example fatty acid polyglycol esters, can be used for controlling foaming. To maintain the stability of plasmids, suitable selective substances, for example antibiotics, can be added to the medium. To maintain aerobic conditions, oxygen or oxygen-containing gas mixtures, for example ambient air, are fed into the culture. The temperature of the culture is normally in the range from 20° C., to 45° C. The culture is continued until a maximum of the desired product has formed. This target is normally reached within 10 hours to 160 hours.

The fermentation broth is then processed further. Depending on requirements, the biomass can be removed from the fermentation broth completely or partially by separation techniques, for example centrifugation, filtration, decanting or a combination of these methods or can be left in it completely.

If the polypeptides are not secreted in the culture medium, the cells can also be lysed and the product can be obtained from the lysate by known methods for isolation of proteins. The cells can optionally be disrupted with high-frequency ultrasound, high pressure, for example in a French press, by osmolysis, by the action of detergents, lytic enzymes or organic solvents, by means of homogenizers or by a combination of several of the aforementioned methods.

The polypeptides can be purified by known chromatographic techniques, such as molecular sieve chromatography (gel filtration), such as Q-sepharose chromatography, ion exchange chromatography and hydrophobic chromatography, and with other usual techniques such as ultrafiltration, crystallization, salting-out, dialysis and native gel electrophoresis. Suitable methods are described for example in Cooper, T. G., Biochemische Arbeitsmethoden [Biochemical processes], Verlag Walter de Gruyter, Berlin, New York or in Scopes, R., Protein Purification, Springer Verlag, New York, Heidelberg, Berlin.

For isolating the recombinant protein, it can be advantageous to use vector systems or oligonucleotides, which lengthen the cDNA by defined nucleotide sequences and therefore code for altered polypeptides or fusion proteins, which for example serve for easier purification. Suitable modifications of this type are for example so-called “tags” functioning as anchors, for example the modification known as hexa-histidine anchor or epitopes that can be recognized as antigens of antibodies (described for example in Harlow, E, and Lane, D., 1988, Antibodies: A Laboratory Manual. Cold Spring Harbor (N.Y.) Press). These anchors can serve for attaching the proteins to a solid carrier, for example a polymer matrix, which can for example be used as packing in a chromatography column, or can be used on a microtiter plate or on some other carrier.

At the same time these anchors can also be used for recognition of the proteins. For recognition of the proteins, it is moreover also possible to use usual markers, such as fluorescent dyes, enzyme markers, which form a detectable reaction product after reaction with a substrate, or radioactive markers, alone or in combination with the anchors for derivatization of the proteins.

### h. Polypeptide Immobilization

The enzymes or polypeptides according to the invention can be used free or immobilized in the method described herein. An immobilized enzyme is an enzyme that is fixed to an inert carrier. Suitable carrier materials and the enzymes immobilized thereon are known from EP-A-1149849, EP-A-1 069 183 and DE-OS 100193773 and from the references cited therein. Reference is made in this respect to the disclosure of these documents in their entirety. Suitable carrier materials include for example clays, clay minerals, such as kaolinite, diatomaceous earth, perlite, silica, aluminum oxide, sodium carbonate, calcium carbonate, cellulose powder, anion exchanger materials, synthetic polymers, such as polystyrene, acrylic resins, phenol formaldehyde resins, polyurethanes and polyolefins, such as polyethylene and polypropylene. For making the supported enzymes, the carrier materials are usually employed in a finely-divided, particulate form, porous forms being preferred. The particle size of the carrier material is usually not more than 5 mm, in particular not more than 2 mm (particle-size distribution curve). Similarly, when using dehydrogenase as whole-cell catalyst, a free or immobilized form can be selected. Carrier materials are e.g. Ca-alginate, and carrageenan. Enzymes as well as cells can also be crosslinked directly with glutaraldehyde (cross-linking to CLEAs). Corresponding and other immobilization techniques are described for example in J. Lalonde and A. Margolin “Immobilization of Enzymes” in K. Drauz and H. Waldmann, Enzyme Catalysis in Organic Synthesis 2002, Vol. III, 991-1032, Wiley-VCH, Weinheim. Further information on biotransformations and bioreactors for carrying out methods according to the invention are also given for example in Rehm et al. (Ed.) Biotechnology, 2nd Edn, Vol 3, Chapter 17, VCH, Weinheim.

### i. Reaction Conditions for Biocatalytic Production Methods of the Invention

The reaction of the present invention may be performed under in vivo or in vitro conditions.

The at least one polypeptide/enzyme which is present during a method of the invention or an individual step of a multistep-method as defined herein above, can be present in living cells naturally or recombinantly producing the enzyme or enzymes, in harvested cells. i.e. under in vivo conditions, or, in dead cells, in permeabilized cells, in crude cell extracts, in purified extracts, or in essentially pure or completely pure form, i.e. under in vitro conditions. The at least one enzyme may be present in solution or as an enzyme immobilized on a carrier. One or several enzymes may simultaneously be present in soluble and/or immobilised form.

The methods according to the invention can be performed in common reactors, which are known to those skilled in the art, and in different ranges of scale, e.g. from a laboratory scale (few millilitres to dozens of litres of reaction volume) to an industrial scale (several litres to thousands of cubic meters of reaction volume). If the polypeptide is used in a form encapsulated by non-living, optionally permeabilized cells, in the form of a more or less purified cell extract or in purified form, a chemical reactor can be used. The chemical reactor usually allows controlling the amount of the at least one enzyme, the amount of the at least one substrate, the pH, the temperature and the circulation of the reaction medium. When the at least one polypeptide/enzyme is present in living cells, the process will be a fermentation. In this case the biocatalytic production will take place in a bioreactor (fermenter), where parameters necessary for suitable living conditions for the living cells (e.g. culture medium with nutrients, temperature, aeration, presence or absence of oxygen or other gases, antibiotics, and the like) can be controlled. Those skilled in the art are familiar with chemical reactors or bioreactors, e.g. with procedures for up-scaling chemical or biotechnological methods from laboratory scale to industrial scale, or for optimizing process parameters, which are also extensively described in the literature (for biotechnological methods see e.g. Crueger und Crueger, Biotechnologie-Lehrbuch der angewandten Mikrobiologie, 2. Ed., R. Oldenbourg Verlag, München, Wien, 1984).

Cells containing the at least one enzyme can be permeabilized by physical or mechanical means, such as ultrasound or radiofrequency pulses. French presses, or chemical means, such as hypotonic media, lytic enzymes and detergents present in the medium, or combination of such methods. Examples for detergents are digitonin, n-dodecylmaltoside, octylglycoside, TritonR: X-100, Tween® 20, deoxycholate, CHAPS (3-[(3-Cholamidopropyl)dimethylammonio]-1-propansulfonate), Nonidet P40 (Ethylphenolpoly(ethyleneglycolether), and the like.

Instead of living cells biomass of non-living cells containing the required biocatalyst(s) may be applied of the biotransformation reactions of the invention as well.

If the at least one enzyme is immobilised, it is attached to an inert carrier as described above.

The conversion reaction can be carried out batch wise, semi-batch wise or continuously. Reactants (and optionally nutrients) can be supplied at the start of reaction or can be supplied subsequently, either semi-continuously or continuously.

The reaction of the invention, depending on the particular reaction type, may be performed in an aqueous, aqueous-organic or non-aqueous reaction medium.

An aqueous or aqueous-organic medium may contain a suitable buffer in order to adjust the pH to a value in the range of 5 to 11, like 6 to 10.

In an aqueous-organic medium an organic solvent miscible, partly miscible or immiscible with water may be applied. Non-limiting examples of suitable organic solvents are listed below. Further examples are mono- or polyhydric, aromatic or aliphatic alcohols, in particular polyhydric aliphatic alcohols like glycerol.

The non-aqueous medium may contain is substantially free of water, i.e. will contain less that about 1 wt.-% or 0.5 wt.-% of water.

Biocatalytic methods may also be performed in an organic non-aqueous medium. As suitable organic solvents there may be mentioned aliphatic hydrocarbons having for example 5 to 8 carbon atoms, like pentane, cyclopentane, hexane, cyclohexane, heptane, octane or cyclooctane: aromatic carbohydrates, like benzene, toluene, xylenes, chlorobenzene or dichlorobenzene, aliphatic acyclic and ethers, like diethylether, methyl-tert.-butylether, ethyl-tert.-buty lether, dipropylether, diisopropylether, dibutylether; or mixtures thereof.

The concentration of the reactants/substrates may be adapted to the optimum reaction conditions, which may depend on the specific enzyme applied. For example, the initial substrate concentration may be in the 0, 1 to 0.5 M, as for example 10 to 100 mM.

The reaction temperature may be adapted to the optimum reaction conditions, which may depend on the specific enzyme applied. For example, the reaction may be performed at a temperature in a range of from 0 to 70° C., as for example 20 to 50 or 25 to 40° C. Examples for reaction temperatures are about 30° C., about 35° C., about 37° C., about 40° C., about 45° C., about 50° C., about 55° C., and about 60° C.

The process may proceed until equilibrium between the substrate and then product(s) is achieved, but may be stopped earlier. Usual process times are in the range from 1 minute to 25 hours, in particular 10 min to 6 hours, as for example in the range from 1 hour to 4 hours, in particular 1.5 hours to 3.5 hours. These parameters are non-limiting examples of suitable process conditions.

If the host is a transgenic plant, optimal growth conditions can be provided, such as optimal light, water and nutrient conditions, for example.

### k. Product Isolation

The methodology of the present invention can further include a step of recovering an end or intermediate product, optionally in stereoisomerically or enantiomerically substantially pure form. The term “recovering” includes extracting, harvesting, isolating or purifying the compound from culture or reaction media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like.

Identity and purity of the isolated product may be determined by known techniques, like High Performance Liquid Chromatography (HPLC), gas chromatography (GC), Spektroskopy (like IR, UV, NMR), Colouring methods, TLC, NIRS, enzymatic or microbial assays. (see for example: Patek et al. (1994) Appl. Environ. Microbiol. 60:133-140: Malakhova et al. (1996) Biotekhnologiya 11 27-32: und Schmidt et al. (1998) Bioprocess Engineer. 19:67-70. Ullmann's Encyclopedia of Industrial Chemistry (1996) Bd. A27, VCH: Weinheim, S. 89-90, S. 521-540, S. 540-547, S. 559-566, 575-581 und S. 581-587: Michal, G (1999) Biochemical Pathways: An Atlas of Biochemistry and Molecular Biology, John Wiley and Sons: Fallon, A. et al. (1987) Applications of HPLC in Biochemistry in: Laboratory Techniques in Biochemistry and Molecular Biology, Bd. 17.)

The cyclic terpene compound produced in any of the method described herein can be converted to derivatives such as, but not limited to hydrocarbons, esters, amides, glycosides, ethers, epoxides, aldehydes, ketons, alcohols, diols, acetals or ketals. The terpene compound derivatives can be obtained by a chemical method such as, but not limited to oxidation, reduction, alkylation, acylation and/or rearrangement. Alternatively, the terpene compound derivatives can be obtained using a biochemical method by contacting the terpene compound with an enzyme such as, but not limited to an oxidoreductase, a monooxygenase, a dioxygenase, a transferase. The biochemical conversion can be performed in-vitro using isolated enzymes, enzymes from lysed cells or in-vivo using whole cells.

### l. Fermentative Production of Terpene/Terpenoid Compounds, Like Labdane Type Compounds

The invention also relates to methods for the fermentative production of terpene/terpenoid compounds like labdane type compounds.

A fermentation as used according to the present invention can, for example, be performed in stirred fermenters, bubble columns and loop reactors. A comprehensive overview of the possible method types including stirrer types and geometric designs can be found in “Chmiel: Bioprozesstechnik: Einführung in die Bioverfahrenstechnik, Band 1”. In the process of the invention, typical variants available are the following variants known to those skilled in the art or explained, for example, in “Chmiel, Hammes and Bailey: Biochemical Engineering”, such as batch, fed-batch, repeated fed-batch or else continuous fermentation with and without recycling of the biomass. Depending on the production strain, sparging with air, oxygen, carbon dioxide, hydrogen, nitrogen or appropriate gas mixtures may be effected in order to achieve good yield (YP/S).

The culture medium that is to be used must satisfy the requirements of the particular strains in an appropriate manner. Descriptions of culture media for various microorganisms are given in the handbook “Manual of Methods for General Bacteriology” of the American Society for Bacteriology (Washington D. C., USA, 1981).

These media that can be used according to the invention may comprise one or more sources of carbon, sources of nitrogen, inorganic salts, vitamins and/or trace elements.

Preferred sources of carbon are sugars, such as mono-, di- or polysaccharides. Very good sources of carbon are for example glucose, fructose, mannose, galactose, ribose, sorbose, ribulose, lactose, maltose, sucrose, raffinose, starch or cellulose. Sugars can also be added to the media via complex compounds, such as molasses, or other by-products from sugar refining. It may also be advantageous to add mixtures of various sources of carbon. Other possible sources of carbon are oils and fats such as soybean oil, sunflower oil, peanut oil and coconut oil, fatty acids such as palmitic acid, stearic acid or linoleic acid, alcohols such as glycerol, methanol or ethanol and organic acids such as acetic acid or lactic acid.

Sources of nitrogen are usually organic or inorganic nitrogen compounds or materials containing these compounds. Examples of sources of nitrogen include ammonia gas or ammonium salts, such as ammonium sulfate, ammonium chloride, ammonium phosphate, ammonium carbonate or ammonium nitrate, nitrates, urea, amino acids or complex sources of nitrogen, such as corn-steep liquor, soybean flour, soy-bean protein, yeast extract, meat extract and others. The sources of nitrogen can be used separately or as a mixture.

Inorganic salt compounds that may be present in the media comprise the chloride, phosphate or sulfate salts of calcium, magnesium, sodium, cobalt, molybdenum, potassium, manganese, zinc, copper and iron.

Inorganic sulfur-containing compounds, for example sulfates, sulfites, di-thionites, tetrathionates, thiosulfates, sulfides, but also organic sulfur compounds, such as mercaptans and thiols, can be used as sources of sulfur.

Phosphoric acid, potassium dihydrogenphosphate or dipotassium hydrogenphosphate or the corresponding sodium-containing salts can be used as sources of phosphorus.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention may also contain other growth factors, such as vitamins or growth promoters, which include for example biotin, riboflavin, thiamine, folic acid, nicotinic acid, pantothenate and pyridoxine. Growth factors and salts often come from complex components of the media, such as yeast extract, molasses, corn-steep liquor and the like. In addition, suitable precursors can be added to the culture medium. The precise composition of the compounds in the medium is strongly dependent on the particular experiment and must be decided individually for each specific case. Information on media optimization can be found in the textbook “Applied Microbiol. Physiology, A Practical Approach” (1997) Growing media can also be obtained from commercial suppliers, such as Standard 1 (Merck) or BHI (Brain heart infusion, DIFCO) etc.

All components of the medium are sterilized, either by heating (20 min at 1.5 bar and 121° C.) or by sterile filtration. The components can be sterilized either together, or if necessary separately. All the components of the medium can be present at the start of growing, or optionally can be added continuously or by batch feed.

The temperature of the culture is normally between 15° C., and 45° C., preferably 25° C. to 40° C., and can be kept constant or can be varied during the experiment. The pH value of the medium should be in the range from 5 to 8.5, preferably around 7.0. The pH value for growing can be controlled during growing by adding basic compounds such as sodium hydroxide, potassium hydroxide, ammonia or ammonia water or acid compounds such as phosphoric acid or sulfuric acid. Antifoaming agents, e.g. fatty acid polyglycol esters, can be used for controlling foaming. To maintain the stability of plasmids, suitable substances with selective action, e.g. antibiotics, can be added to the medium. Oxygen or oxygen-containing gas mixtures, e.g. the ambient air, are fed into the culture in order to maintain aerobic conditions.

The temperature of the culture is normally from 20° C., to 45° C. Culture is continued until a maximum of the desired product has formed. This is normally achieved within 1 hour to 160 hours.

The methodology of the present invention can further include a step of recovering said terpene alcohol.

The term “recovering” includes extracting, harvesting, isolating or purifying the compound from culture media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like.

Before the intended isolation the biomass of the broth can be removed. Processes for removing the biomass are known to those skilled in the art, for example filtration, sedimentation and flotation. Consequently, the biomass can be removed, for example, with centrifuges, separators, decanters, filters or in flotation apparatus. For maximum recovery of the product of value, washing of the biomass is often advisable, for example in the form of a diafiltration. The selection of the method is dependent upon the biomass content in the fermenter broth and the properties of the biomass, and also the interaction of the biomass with the product of value.

In one embodiment, the fermentation broth can be sterilized or pasteurized. In a further embodiment, the fermentation broth is concentrated. Depending on the requirement, this concentration can be done batch wise or continuously. The pressure and temperature range should be selected such that firstly no product damage occurs, and secondly minimal use of apparatus and energy is necessary. The skillful selection of pressure and temperature levels for a multistage evaporation in particular enables saving of energy.

The following examples are illustrative only and are not intended to limit the scope of the embodiments an embodiments described herein.

The numerous possible variations that will become immediately evident to a person skilled in the art after heaving considered the disclosure provided herein also fall within the scope of the invention.

### Experimental Part

The invention will now be described in further detail by way of the following Examples.

### A) MATERIALS

Unless otherwise stated, all chemical and biochemical materials and microorganisms or cells employed herein are commercially available products.

Unless otherwise specified, recombinant proteins are cloned and expressed by standard methods, such as, for example, as described by Sambrook, J., Fritsch, E. F, and Maniatis, T., Molecular cloning: A Laboratory Manual, 2nd Edition, Cold Spring Harbor Laboratory, Cold Spring Harbor Laboratory Press, Cold Spring Harbor, NY, 1989.

### B) GENERAL METHODS

### Cell Free Protein Fractions Preparation.

The expression vectors were transformed into E. coli KRX cells (Promega Corporation, Madison, WI, USA) and the transformed cells were selected on LB medium plates supplemented with the appropriate antibiotic. The cells were then grown in 25 mL liquid LB medium supplemented with the appropriate antibiotic at 37° C., to an OD of 1. The expression of the recombinant proteins was induced with 1 mM isopropyl-1-thio-β-D-galactopyranoside and 0.1% (w/v) L-rhamnose monohydrate, and the cells were incubated 24 hours at 25° C. with moderate shaking.

The bacterial cells were harvested by centrifugation (5000 g, 12 min) and disrupted by sonication (Sonics, Vibra cell X 130 sonicator equipped with a 6 mm diameter tip microprobe; 3 times 20 second 20 kHz pulses at 80% of maximum power) on ice, in 1.8 mL of 50 mM MOPSO buffer pH 7.4 containing 15% glycerol. The lysates were cleared by centrifugation (3500 g, 8 min, 4° C.) and the resulting supernatants were stored frozen and used as the enzyme source for in vitro assays.

### In Vitro Enzyme Assays.

The protein fractions containing one of the recombinant proteins was incubated 4 hours at 24° C. with shaking at 230 rpm in assays consisting of 20 μl of cell-free extract, 160 to 320 mg/L of substrate (using a 40 g/L substrate stock solution in DMSO), 1 mM of cofactor whenever relevant, and 50 mM MOPSO pH 7.4 in a final volume of 0.5 to 1 mL in borosilicate glass and PTFE sealed screw-capped tubes (11 mL capacity) (Wheaton, Millville, NJ 08332 USA). Assays were extracted with 1 volume of methyl-tert-butyl-ether (MTBE) and analyzed by GC-MS as described below.

### Whole-Cell Bioconversion Assays.

Bioconversions of compounds were performed using E. coli cells expressing recombinant enzymes. The expression vectors are transformed into E. coli KRX cells (Promega Corporation, Madison, WI, USA) and the transformed cells were selected on LB medium plates supplemented with the appropriate antibiotic. The cells were first cultivated overnight at 30° C. in 5 mL LB medium supplemented 1% glucose and with the appropriate antibiotic. The next day, 20 mL of TB medium (Terrific Broth) supplemented with the appropriate antibiotic were inoculated with an initial optical density of 0.2 to 0.75. The culture were incubated in shake flasks at 37° C. until an optical density of 1 to 4 was reached and the expression of the recombinant proteins was induced by the addition of 0.1 mM isopropyl-1-thio-β-D-galactopyranoside IPTG and 0.1% rhamnose. The cultures were then distributed in 0.5 to 1 mL aliquotes in 12 mL glass tubes and incubated at 20° C. with moderate shaking.

The substrate was added to each tube 90 minutes after induction of the expression of the recombinant protein. The substrate was either added to a final concentration of 0.25 to 1 g/L using a 40 g/L stock solution in DMSO. Alternatively, an emulsion was prepared containing 150 mg/mL of Tween®: 80 (Sigma-Aldrich) and 300 mg/mL of substrate in water and added to the assays to reach a final concentration of 12 mg/mL of substrate.

After 8 to 48 hours of incubation, the cultures were extracted with one volume of MTBE and analyzed by GC-MS as described below.

### Cultivation of Engineered Bacteria Cells Under Conditions Enabling Production of Terpene Compounds.

The DP1205 E. coli cells were transformed with one or two expression plasmids carrying terpene biosynthesis genes and/or terpene modification enzymes and the transformed cells were cultured with the appropriate antibiotics (kanamycin (50 μg/mL) and/or chloramphenicol (34 μg/mL)) on LB-agarose plates. Single colonies were used to inoculate 5 mL liquid LB medium supplemented with the same antibiotics, 4 g/L glucose and 10% (v/v) dodecane. The next day 2 mL of TB medium supplemented with the same antibiotics and 10% (v/v) dodecane were inoculated with 0.2 mL of the overnight culture. The cultures were incubated at 37° C. until an optical density of 3 was reached. The expression of the recombinant proteins was then induced by addition of 1 mM IPTG and the cultures were incubated for 72 h at 20° C.

The cultures were then extracted with one volume of (MTBE) and the composition of the organic phase was analyzed by GC-MS as described below. For quantification an internal standard (α-longipinene (Aldrich)) was added to the extract prior to GC-MS analysis and concentrations of the components were estimated based on comparison of the peak areas.

### GC-MS Analysis Methods.

Samples of whole cell bioconversion assays were analyzed using an Agilent 7890A GC system coupled with a 5975C series Mass Selective Detector (MSD) and equipped with a split/splitless injector (Agilent Technologies, CA).

The GC inlet temperature was set to 230° C., and 1.0 μL of sample was injected in split mode (split ratio 20:1) and analyzed on a DB-5 ms capillary column (30 m×0.25 mm inner diameter×0.25 μm film thickness; Agilent J&W) using helium as a carrier gas at a constant flow of 1 mL/min. The initial temperature of the oven was set at 80° C., and was programmed to 240° C. (10° C./min; hold 1 min) and then to 300° C. (20° C./min; hold 1 min).

Samples of in vitro assays were analyzed using an Agilent 6890N GC system coupled with a 5975 series Mass Selective Detector (MSD) and equipped with a split/splitless injector (Agilent Technologies, CA) and a CombiPAL autosampler (CTC Analytics, Zwingen, Switzerland) injection system. The GC inlet temperature was set to 250° C., and 1.0 μL of sample was injected in pulsed-splitless mode (pulse pressure 1.56 bar, pulse time 0.6 min) and analyzed on a DB-Ims capillary column (30 m×0.25 mm inner diameter×0.25 μm film thickness; Agilent J&W) using helium as a carrier gas at a constant flow of 1.2 mL/min. The initial temperature of the oven was set at 100° C. (hold 1 min) and was programmed to 260° C. (10 to 20° C./min) and then to 300° C. (30° C./min; hold 1 min). For smaller molecular mass compounds, the same conditions were used for analysis except that the oven initial temperature was lowered down to 80° C.

### Engineering of Recombinant Strains for Degradation of Terpene Compounds.

Recombinant strains capable of producing or converting compounds were engineered by introducing nucleotide sequences encoding for one or more of the following proteins:
a Baeyer-Villiger monooxygenase (BVMO) selected from
SCH23-BVMO1 from Hyphozyma roseonigra (SEQ ID NO: 2),
SCH24-BVMO1 from Filobasidium magnum (SEQ ID NO: 6),
SCH25-BVMO1 from Papiliotrema laurentii (SEQ ID NO: 10), and
SCH46-BVMO1 from Bensingtonia ciliata (SEQ ID NO: 13);
an esterase selected from
SCH23-EST from Hyphozyma roseonigra (SEQ ID NO: 20),
SCH24-EST from Filobasidium magnum (SEQ ID NO: 24),
SCH25-EST from Papiliotrema laurentii (SEQ ID NO: 28); and
an enal-cleaving enzyme (lyase) selected from
SCH94-3944 Rhodococcus erythropolis (SEQ ID NO: 34),
SCH80-05241 Rhodococcus rhodochrous (SEQ ID NO: 38),
Pdigit7033 Penicillium digitatum (SEQ ID NO: 42),
PitalDUF4334-1 Penicillium italicum (SEQ ID NO: 46),
AspWeDUF4334 Aspergillus wentii (SEQ ID NO: 49),
RhoagDUF4334-2 Rhodococcus hoagii strain PAM2288 (SEQ ID NO: 53),
RhoagDUF4334-3 Rhodococcus hoagii strain N128 (SEQ ID NO: 56),
RhoagDUF4334-4 Rhodococcus hoagii NBRC 10125 (SEQ ID NO: 59),
CnecaDUF4334 Cupriavidus necator (SEQ ID NO: 62),
Rins-DUF4334 Ralstonia insidiosa (SEQ ID NO: 69),
CgatDUF4334 Cryptococcus gattii EJB2 (SEQ ID NO: 72),
GclavDUF4334 Grosmannia clavigera kw1407 (SEQ ID NO: 75),
TcurvaDUF4334 Thermomonospora curvata (SEQ ID NO:81),
PprotDUF4334 Pseudomonas protegens (SEQ ID NO: 87),

Bacterial host cells for in vitro enzyme assays or whole cell bioconversion assays were selected from E. coli KRX cells (Promega Corporation, Madison, WI, USA) and E. coli BL21 Star™ (DE3) cells (ThermoFisher).

For the biochemical production of terpene compounds using one or more enzyme(s) selected from the enzymes listed above, the host cell was engineered to produce increased amounts of farnesyl-pyrophosphate (FPP) using a mevalonate enzyme pathway and was further transformed to express sesquiterpene or diterpene biosynthesis enzymes.

Engineering of a Recombinant E. coli Strain for Production of FPP by Chromosomal Integration of the Genes Encoding Mevalonate Pathway Enzymes.

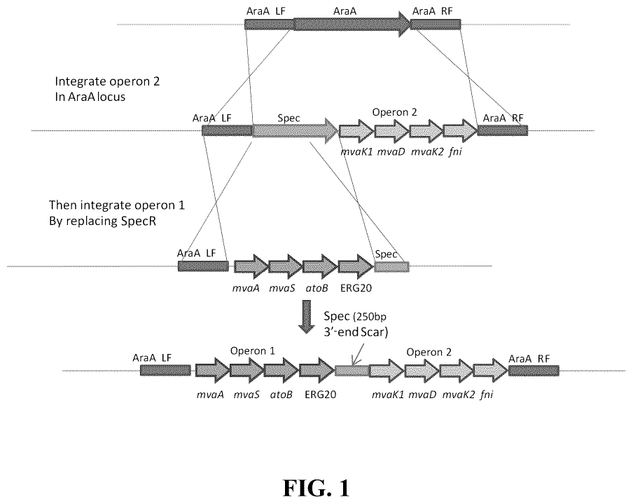

An E. coli strain was engineered to produce farnesyl-pyrophosphate (FPP) by chromosomal integration of recombinant genes encoding mevalonate pathway enzymes. See also construction scheme and recombination events depicted in FIG. 1.

An upper pathway operon (operon 1 from acetyl-CoA to mevalonate) was designed consisting of the atoB gene from E. coli encoding an acetoacetyl-CoA thiolase, and the mvaA and mvaS genes from Staphylococcus aureus encoding a HMG-COA synthase and a HMG-CoA reductase, respectively.

As a lower mevalonate pathway operon (operon 2 from mevalonate to farnesyl pyrophosphate), a natural operon from the gram-negative bacteria Streptococcus pneumoniae was selected, encoding a mevalonate kinase (mvaK1), a phosphomevalonate kinase (mvaK2), a phosphomevalonate decarboxylase (mvaD), and an isopentenyl diphosphate isomerase (fni).

A codon optimized Saccharomyces cerevisiae FPP synthase encoding gene (ERG20) was introduced at the 3′-end of the upper pathway operon to convert isopentenyl-diphosphate (IPP) and dimethylallyl-diphosphate (DMAPP) into FPP.

The above described operons were synthesized by DNA 2.0 and integrated into the araA gene of the Escherichia coli strain BL21 (DE3). The heterologous pathway was introduced in two separate recombination steps using the CRISPR/Cas9 genome engineering system. The first operon (lower pathway: operon 2) to be integrated carries a spectinomycin (Spec) marker which was used to screen for Spec resistant candidate integrants. The second operon was designed to displace the Spec marker of the previously integrated operon and was accordingly screened for Spec candidate integrants following the second recombination event (see FIG. 1). Guide RNA expression vectors targeting the araA gene were designed and synthetized by DNA 2.0. PCR was used to verify operon integration by designing PCR primers to amplify across the araA gene integration target and across recombination junctions of integrants. One clone yielding correct PCR results was then fully sequenced and archived as strain DP1205.

### Engineering of Recombinant Bacterial Cells for the Production of Copalol.

An operon was constructed containing two cDNAs encoding for:
AspWeTPP, a protein with terpenyl diphosphate phosphatase activity from Aspergillus wentii (SEQ ID NO: 170) (GenBank accession OJJ34585.1) having the ability to dephosphory late terpenyl diphosphate compounds, like copalyl PP; and
PvCPS, a protein having prenyl-transferase and copalyl-diphosphate synthase activites from Talaromyces verruculosus (SED ID NO: 173) (GenBank accession BBF88128.1). PvCPS catalyzes the production of copalyl PP from IPP and DMAPP.

The cDNAs encoding for AspWeTPP and PvCPS were codon optimized (SEQ ID NOs: 171 and 174). An operon was designed containing the two cDNAs and an RBS sequence (AAGGAGGTAAAAAA) (SEQ ID NO: 196) placed upstream of the cDNAs. The operon was synthesized and cloned into the pJ401 expression plasmid (ATUM, Newark, California) providing the plasmid pJ401-CPOL-4.

Transformation of E. coli cells such as the DP1205 E. coli cells with the plasmid pJ401-CPOL-4 provides recombinant cells capable of producing copalol when cultivated under conditions enabling production of terpene compounds.

### Engineering of Recombinant Bacteria Cells for the Production of Copalal.

An operon was constructed containing 3 cDNAs encoding for:
AspWeTPP, a protein with terpenyl diphosphate phosphatase activity from Aspergillus wentii (SEQ ID NO: 170) (GenBank accession OJJ34585.1) having the ability to dephosphory late terpenyl diphosphate compounds, like copalyl PP;
AzTolADH1, a protein with alcohol dehydrogenase (ADH) activity from Azoarcus toluclasticus (SEQ ID NO: 167) (GenBank accession WP_018990713.1), having the ability to oxidize terpene alcohols like copalol to the respective carbonyl compound like copalal; and
PvCPS, a protein having prenyl-transferase and copalyl-diphosphate synthase activities from Talaromyces verruculosus (SEQ ID NO: 173) (GenBank accession BBF88128.1) having the ability to produce cyclic terpenyl diphosphate compounds, like copalyl diphosphate, from IPP and DMAPP.

The cDNAs encoding for AspWeTPP, AzTolADH1 and PvCPS were codon optimized (SEQ ID NOs: 171, 168 and 174). An operon was designed containing successively the three cDNAs and an RBS sequence (AAGGAGGTAAAAAA) (SEQ ID NO: 196) placed upstream of each cDNA. The operon was synthesized and cloned into the pJ401 expression plasmid (ATUM, Newark, California) providing the plasmid pJ401-CPAL-1.

Transformation of E. coli cells such as the DP1205 E. coli cells with the plasmid pJ401-CPAL-1 provides recombinant cells capable of producing copalal when cultivated under conditions enabling production of terpene compounds.

### Engineering of Recombinant Bacteria Cells for the Production of Farnesal.

An operon was constructed containing two cDNAs encoding for:
TalCeTPP, a protein with terpenyl diphosphate phosphatase activity from Talaromyces cellulolyticus (GenBank: GAM42000.1) (SEQ ID NO: 176) having the ability to dephosphory late terpenyl diphosphate compounds, like farnesyl diphosphate; and
CdGeoA, a protein with alcohol dehydrogenase (ADH) activity from Castellaniella defragrans (NCBI accession WP_043683915.1) (SEQ ID NO: 179) having the ability to oxidize terpene alcohols like farnesol to the respective carbonyl compound like farnesal.

The cDNAs encoding for TalCeTPP and CdGeoA were codon optimized (SEQ ID NOs: 177 and 180). An operon was designed containing successively the two cDNAs and an RBS sequence (AAGGAGGTAAAAAA) (SEQ ID NO: 196) placed upstream of each cDNA. The operon was synthesized and cloned into the pJ401 expression plasmid (ATUM, Newark, California) providing the plasmid pJ401-FAL-1.

Transformation of E. coli cells such as the DP1205 E. coli cells with the plasmid pJ401-FAL-1 provides recombinant cells capable of producing farnesal when cultivated under conditions enabling production of terpene compounds.

### Engineering of Recombinant Bacteria Cells for the Production of Labdendiol.

An operon was constructed containing three cDNAs encoding for:
TalVeTPP, a protein with terpenyl diphosphate phosphatase activity from Talaromyces verruculosus (Genbank accession KUL89334.1) (SEQ ID NO: 194); having the ability to dephosphory late terpenyl diphosphate compounds, like labdenediol PP
SsLPS, a protein with labdendiol-phyrophosphate (LPP) synthase activity from Salvia sclarea (Genbank accession AET21247.1) (SEQ ID NO: 188) having the ability to produce cyclic terpenyl diphosphate compounds, like labdenediol diphosphate, from GGPP; and
CrtE, a geranylgeranyl-diphosphate synthase from Pantoea agglomerans (GenBank accession AAA24819.1) (SEQ ID NO: 191) having the ability to produce GGPP from FPP.

The cDNAs encoding for TalVeTPP, SsLPS and CrtE were codon optimized (SEQ ID NOs: 195, 189 and 192). An operon was designed containing successively the three cDNAs and an RBS sequence (AAGGAGGTAAAAAA) (SEQ ID NO: 196) placed upstream of each cDNA. The operon was synthesized and cloned in the pJ401 expression plasmid (ATUM, Newark, California) providing the plasmid pJ401-LOH-2.

Transformation of E. coli cells such as the DP1205 E. coli cells with the plasmid pJ401-LOH-2 provides recombinant cells capable of producing labdendiol when cultivated under conditions enabling production of terpene compounds.

### Transformation, Selection and Cultivation of Yeast Cells.

All yeast cell transformations were performed with the lithium acetate protocol as described in Gietz and Woods, Methods Enzymol., 2002, 350:87-96. Transformation mixtures were plated on SmUra- or SmLeu-media plates containing 6.7 g/L of Yeast Nitrogen Base without amino acids (BD Difco, New Jersey, USA), 1.92 g/L Dropout supplement without uracil (Sigma Aldrich, Missouri, USA) or 1.6 g/L Dropout supplement without leucine (Sigma Aldrich, Missouri, USA), 20 g/L glucose and 20 g/L agar. Plates were incubated for 3-4 days at 30° C.)

### Engineering of Yeast Cells for an Increased Level of Endogenous Farnesyl-Diphosphate.

To increase the level of endogenous farnesyl-diphosphate (FPP) pool in S. cerevisiae cells, an extra copy of all yeast endogenous genes involved in the mevalonate pathway, from ERG10 coding for acetyl-CoA C-acetyltransferase to ERG20 coding for FPP synthetase, were integrated into the genome of the S. cerevisiae strain CEN.PK2-1C (Euroscarf, Frankfurt, Germany) under the control of galactose-inducible promoters, similarly as described in Paddon et al., Nature, 2013, 496:528-532. Briefly, three cassettes were integrated in the LEU2, TRP1 and URA3 loci respectively. A first cassette contained the genes ERG20 and a truncated HMG1 (tHMG1 as described in Donald et al., Proc Natl Acad Sci USA, 1997, 109: E111-8) under the control of the bidirectional promoter of GAL10/GAL1 and the genes ERG19 and ERG13 also under the control of the GAL10 GAL1 promoter. The cassette was flanked by two 100 nucleotides regions corresponding to the up- and down-stream sections of LEU2. A second cassette contained the genes IDI1 and tHMG1 which were under the control of the GAL10/GAL1 promoter and the gene ERG13 under the control of the promoter region of GAL7. The cassette was flanked by two 100 nucleotides regions corresponding to the up- and down-stream sections of TRP1. A third cassette contained the genes ERG10, ERG12, tHMG1 and ERG8, all under the control of GAL10/GAL1 promoters. The cassette was flanked by two 100 nucleotides regions corresponding to the up- and down-stream sections of URA3. All genes in the three cassettes included 200 nucleotides of their own terminator regions. Also, an extra copy of GAL4 under the control of a mutated version of its own promoter, as described in Griggs and Johnston, Proc Natl Acad Sci USA, 1991, 88:8597-8601, was integrated upstream of the ERG9 promoter region. In addition, the expression of ERG9 was modified by promoter exchange. The GAL7, GAL10 and GAL1 genes were deleted using a cassette containing the HIS3 gene with its own promoter and terminator. The resulting strain was mated with the strain CEN.PK2-1D (Euroscarf, Frankfurt, Germany) obtaining a diploid strain termed YST045 which was induced for sporulation according to Solis-Escalante et al., FEMS Yeast Res, 2015, 15:2. Spore separation was achieved by resuspension of asci in 200 μL 0.5M sorbitol with 2 μL zymolyase (1000 U mL 1. Zymo research, Irvine, CA) and incubation at 37° C. for 20 minutes. The mixture was then plated on media containing 20 g/L peptone, 10 g/L yeast extract and 20 g/L agar, and one germinated spore was isolated and termed YST075.

### Engineering of Recombinant Yeast Cells for the Production of Copalol.

For copalol production, expression of the GGPP synthase carG (from Blakeslea trispora, NCBI accession JQ289995.1) (SEQ ID NO: 182), the copalyl-pyrophosphate synthase SmCPS2 (from Salvia miltiorrhiza, NCBI accession ABV57835.1) (SEQ ID NO: 185) and the copalyl-pyrophosphate phosphatase TalVeTPP (from Talaromyces verruculosus, NCBI accession KUL89334.1) (SEQ ID NO: 194) in the different engineered yeast cells was achieved with a plasmid system constructed in vivo using yeast endogenous homologous recombination as previously described in Kuijpers et al., Microb Cell Fact., 2013, 12:47. The plasmid is composed of six DNA fragments which were used for S. cerevisiae co-transformation. The fragments were:
a) LEU2 yeast marker, constructed by PCR using the primers 5′-AGGTGCAGTTCGCGTGCAATTATAACGTCGTGGCAACTGTTATCAGTCG TACCGCGCCATTCGACTACGTCGTAAGGCC-3′ (SEQ ID NO: 124) and 5′-TCGTGGTCAAGGCGTGCAATTCTCAACACGAGAGTGATTCTTCGGCGTT GTTGCTGACCATCGACGGTCGAGGAGAACTT-3′ (SEQ ID NO: 125) with the plasmid pESC-LEU (Agilent Technologies, California, USA) as template;
b) AmpR E. coli marker, constructed by PCR using the primers 5′-TGGTCAGCAACAACGCCGAAGAATCACTCTCGTGTTGAGAATTGCACG CCTTGACCACGACACGTTAAGGGATTTTGGTCATGAG-3′ (SEQ ID NO: 126) and 5′-AACGCGTACCCTAAGTACGGCACCACAGTGACTATGCAGTCCGCACTTT GCCAATGCCAAAAATGTGCGCGGAACCCCTA-3′ (SEQ ID NO: 127) with the plasmid pESC-URA as template;
c) Yeast origin of replication, obtained by PCR using the primers 5′-TTGGCATTGGCAAAGTGCGGACTGCATAGTCACTGTGGTGCCGTACTTA GGGTACGCGTTCCTGAACGAAGCATCTGTGCTTCA-3′ (SEQ ID NO: 128) and 5′-CCGAGATGCCAAAGGATAGGTGCTATGTTGATGACTACGACACAGAAC TGCGGGTGACATAATGATAGCATTGAAGGATGAGACT-3′ (SEQ ID NO: 129) with pESC-URA as template;
d) E. coli replication origin, obtained by PCR using the primers 5′-ATGTCACCCGCAGTTCTGTGTCGTAGTCATCAACATAGCACCTATCCTT TGGCATCTCGGTGAGCAAAAGGCCAGCAAAAGG-3′ (SEQ ID NO: 130) and 5′-CTCAGATGTACGGTGATCGCCACCATGTGACGGAAGCTATCCTGACAGT GTAGCAAGTGCTGAGCGTCAGACCCCGTAGAA-3′ (SEQ ID NO: 131) with the plasmid pESC-URA as template;
e) a fragment composed by the last 60 nucleotides of the fragment “d”, 200 nucleotides downstream the stop codon of the yeast gene PGK1, the GGPP synthase coding sequence carG, the bidirectional yeast promoter of GAL10/GAL1, the coding sequence of TalVeTPP, 200 nucleotides downstream the stop codon of the yeast gene CYC1 and the sequence 5′-ATTCCTAGTGACGGCCTTGGGAACTCGATACACGATGTTCAGTAGACCG CTCACACATGG-3′ (SEQ ID NO: 132), this fragment was obtained by DNA synthesis (ATUM, Menlo Park, CA 94025) and
f) a fragment composed by the last 60 nucleotides of fragment “e”, 200 nucleotides downstream the stop codon of the yeast gene CYC1, the SmCPS2 copalyl-pyrophosphate synthase coding sequence, the bidirectional yeast promoter of GAL10/GAL1 and 60 nucleotides corresponding to the beginning of the fragment “a”, this fragment was obtained by DNA synthesis (ATUM, Menlo Park, CA 94025).

Optionally, the GGPP synthase carG and the copalyl-pyrophosphate synthase were replaced by the bi-functional PvCPS.

### Engineering of Recombinant Yeast Cells for the Production of Manooloxy.

For degradation of copalol to manooloxy using different alcohol dehydrogenases (ADHs), Baeyer-Villiger monooxygenases (BVMOs) and esterases (ESTs), genome integrations in the strain YST075 were performed. Each integration cassette was formed by four fragments:
1) A fragment containing 658 bp corresponding to the upstream section of the NDT80 gene and the sequence 5′-GCAGGCAGCTCCATTTCATGTAGGTGATTTATCCCTCCGGGCGGTATTT GAGACTCTCGG-3′ (SEQ ID NO: 121), this fragment was obtained by PCR with genomic DNA from the strain YST075 as template;
2) a fragment containing the sequence 5′-GCAGGCAGCTCCATTTCATGTAGGTGATTTATCCCTCCGGGCGGTATTT GAGACTCTCGG-3′ (SEQ ID NO: 121), the CYC1 terminator region, one of the genes coding for a BVMO, the intergenic region between GAL1 and GAL10 genes, one of the genes encoding for an esterase, the terminator region of the ADH1 gene and the sequence 5′-ACTGCTGGGTACTGTTCAGGCACGATAGGAAATGCGTCCAGCGCATAC ACCAGTCTTAGC-3′ (SEQ ID NO: 122), this fragment was obtained by DNA synthesis (ATUM, Menlo Park, CA 94025),
3) a fragment containing the sequence 5′-ACTGCTGGGTACTGTTCAGGCACGATAGGAAATGCGTCCAGCGCATAC ACCAGTCTTAGC-3′ (SEQ ID NO: 122), the PGK1 terminator region, one of the genes encoding for an alcohol dehydrogenase, the promoter region of the genes GAL1 and GAL10, one of the genes encoding an alcohol dehydrogenase, the CYC1 terminator region and the sequence 5′-AGTCGACCTTACAGCGCCTGGGACTCTACATAAACATGCAGCGAACAT GCTTTCCAACGC-3′ (SEQ ID NO: 123), This fragment might contain one or two alcohol dehydrogenase depending on the experiment performed. They were obtained by DNA synthesis (ATUM, Menlo Park, CA 94025); and
4) a fragment containing the sequence 5′-AGTCGACCTTACAGCGCCTGGGACTCTACATAAACATGCAGCGAACAT GCTTTCCAACGC-3′ (SEQ ID NO: 123), and 405 bp corresponding to the NDT80 gene. This fragment was obtained by PCR with genomic DNA from the strain YST075 as template.

### Engineering of Recombinant Yeast Cells for the Degradation of Copalol to Manooloxy.

For degradation of copalol to manooloxy, using an alcohol dehydrogenase and different enal-cleaving polypeptides, genome integrations in the strain YST075 were performed, each integration cassette was formed by three fragments:
1) A fragment containing 658 bp corresponding to the upstream section of the NDT80 gene and the sequence 5′-GCAGGCAGCTCCATTTCATGTAGGTGATTTATCCCTCCGGGCGGTATTTGAGACT CTCGG-3′ (SEQ ID NO: 121), this fragment was obtained by PCR with genomic DNA from the strain YST075 as template;
2) a fragment containing the sequence 5′-GCAGGCAGCTCCATTTCATGTAGGTGATTTATCCCTCCGGGCGGTATTTGAGACT CTCGG-3′ (SEQ ID NO: 121), the intergenic region between GAL1 and GAL10 genes, one of the genes encoding for an enal-cleaving polypeptide, the terminator region of the ADH1 gene and the sequence 5′-ACTGCTGGGTACTGTTCAGGCACGATAGGAAATGCGTCCAGCGCATACACCAGT CTTAGC-3′ (SEQ ID NO: 122), this fragment was obtained by DNA synthesis (ATUM, Menlo Park, CA 94025); and
3) a fragment containing the sequence 5′-ACTGCTGGGTACTGTTCAGGCACGATAGGAAATGCGTCCAGCGCATACACCAGT CTTAGC-3′ (SEQ ID NO: 122), the PGK1 terminator region, the gene coding for an alcohol dehydrogenase, the promoter region of the genes GAL1 and GAL10, the sequence 5′-AGTCGACCTTACAGCGCCTGGGACTCTACATAAACATGCAGCGAACATGCTTTCC AACGC-3′ (SEQ ID NO: 123) and 405 bp corresponding to the NDT80 gene. This fragment was obtained by DNA synthesis (ATUM, Menlo Park, CA 94025).

In all cases, copalol production was achieved by expressing the biosynthetic pathway in a plasmid system as described above.

### Engineering of Recombinant Yeast Cells for the Production of Gamma-Ambryl Acetate.

For degradation of copalol to gamma-ambryl acetate using an alcohol dehydrogenase, an enal-cleaving polypeptide and different Baeyer-Villiger monooxygenases (BVMOs), genome integrations in the strain YST075 were performed; each integration cassette was formed by three fragments:
(1) A fragment containing 658 bp corresponding to the upstream section of the NDT80 gene and the sequence 5′-GCAGGCAGCTCCATTTCATGTAGGTGATTTATCCCTCCGGGCGGTATTTGAGACT CTCGG-3′ (SEQ ID NO: 121), this fragment was obtained by PCR with genomic DNA from the strain YST075 as template;
(2) a fragment containing the sequence 5′-GCAGGCAGCTCCATTTCATGTAGGTGATTTATCCCTCCGGGCGGTATTTGAGACT CTCGG-3′ (SEQ ID NO: 121), the terminator region of the CYC1 gene, one of the genes coding for the tested BVMOs, the intergenic region between GAL1 and GAL10 genes, the gene encoding for an enal-cleaving polypeptide, the terminator region of the ADH1 gene and the sequence 5′-ACTGCTGGGTACTGTTCAGGCACGATAGGAAATGCGTCCAGCGCATACACCAGT CTTAGC-3′ (SEQ ID NO: 122), this fragment was obtained by DNA synthesis (ATUM, Menlo Park, CA 94025); and
(3) a fragment containing the sequence 5′-ACTGCTGGGTACTGTTCAGGCACGATAGGAAATGCGTCCAGCGCATACACCAGT CTTAGC-3′ (SEQ ID NO: 122), the PGK1 terminator region, the gene coding for an alcohol dehydrogenase, the promoter region of the genes GAL1 and GAL10, the sequence 5′-AGTCGACCTTACAGCGCCTGGGACTCTACATAAACATGCAGCGAACATGCTTTCC AACGC-3′ (SEQ ID NO: 123) and 405 bp corresponding to the NDT80 gene. This fragment was obtained by DNA synthesis (ATUM, Menlo Park, CA 94025).

In all cases, copalol production was achieved by expressing the biosynthetic pathway in a plasmid system as described above.

### Engineering of Recombinant Yeast Cells for the Production Gamma-Ambrol.

For degradation of copalol to gamma-ambrol using an alcohol dehydrogenase, an enal-cleaving polypeptide, a Baeyer-Villiger monooxygenases (BVMOs) and different esterases (EST), genome integrations in the strain YST075 were performed; each integration cassette was formed by four overlapping fragments:
1) A fragment containing at least 300 bp corresponding to the upstream section of the BUD9 gene and at least 60 bp overlapping sequence for in vivo assembly. This fragment was obtained by PCR with genomic DNA from the strain YST075 as template;
2) a fragment containing the terminator region of the ADH1 gene, one of the genes coding for the tested esterases and the intergenic region between GAL1 and GAL10 genes. The fragment was flanked by sequences allowing in vivo assembly. This fragment was obtained by DNA synthesis (ATUM, Menlo Park, CA 94025);
3) a fragment containing the URA3 yeast marker with its own promoter and terminator, flanked by sequences to allow homologous recombination. This fragment was obtained by PCR; and
4) a fragment containing at least 300 bp corresponding to the downstream section of the BUD9 gene and at least 60 bp overlapping sequences to allow in vivo assembly. This fragment was obtained by PCR with genomic DNA from the strain YST075 as template.

In all cases, copalol production was achieved by expressing the biosynthetic pathway in a plasmid system as described above.

### Cultivation of Engineered Yeast Cells Under Conditions Enabling the Production of Terpene Compounds and GC-MS Analysis Methods.

Evaluation of the production of terpenes and derivatives from engineered yeast cells was achieved by culturing cells under conditions similarly as described in Westfall et al., Proc Natl Acad Sci USA, 2012, 109: E111-118 with 10% dodecane or 10% isopropyl myristate (IPM) as organic overlay. The cultures were then extracted with two volumes of MTBE and the composition of the organic phase was analyzed by GC-MS using an Agilent 7890A GC system coupled with a 5975C series Mass Selective Detector (MSD) and equipped with a split/splitless injector and a GC Injector 80 injection system (Agilent Technologies, CA). The GC inlet temperature was set to 260° C., and 1.0 μl of sample was injected in splitless mode and analyzed on a HP-5 GC column (30 m×0.25 mm×0.25 μm; Agilent J&W) using helium as a carrier gas at a constant flow of 1.2 mL/min. The initial temperature of the oven was set at 100° C., and was programmed to 300° C. (10° C./min).

### C) EXAMPLES

### Example 1: In-Vivo-Conversion of Manooloxy to Gamma-Ambryl Acetate Using BVMOs

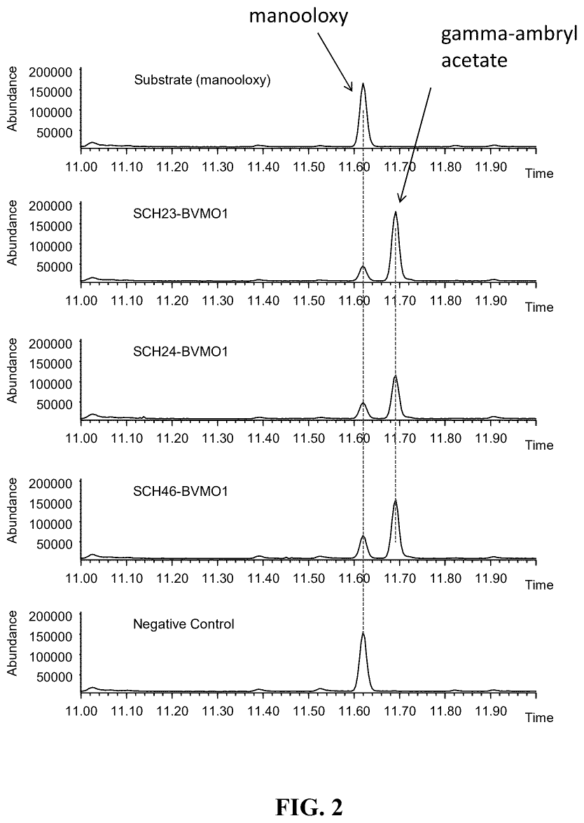

Codon optimized cDNAs encoding for SCH23-BVMO1 from Hyphozyma roseonigra (SEQ ID NO: 2), SCH24-BVMO1 from Filobasidium magnum (SEQ ID NO: 6) and SCH46-BVMO1 from Bensingtonia ciliata (SEQ ID NO: 13) were synthesized and cloned in the pJ414 expression plasmid (ATUM, Newark, California) providing the plasmids pJ414-SCH23-BVMO1, pJ414-SCH24-BVMO1 and pJ414-SCH46-BVMO1. KRX E. coli cells (Promega Corporation, Madison, WI, USA) were transformed with these expression plasmids. The transformed cells were grown and used in whole cell bioconversion assay as described above using manooloxy as substrate. A negative control was included consisting of the cells transformed with an empty plasmid. In the presence of the SCH23-BVMO1, SCH24-BVMO1 or SCH46-BVMO1 recombinant proteins, conversion of manooloxy to gamma-ambryl acetate was observed (FIG. 2). No conversion was observed in the negative control. This experience shows that SCH23-BVMO1, SCH24-BVMO1 or SCH46-BVMO1 can catalyse the following conversion:

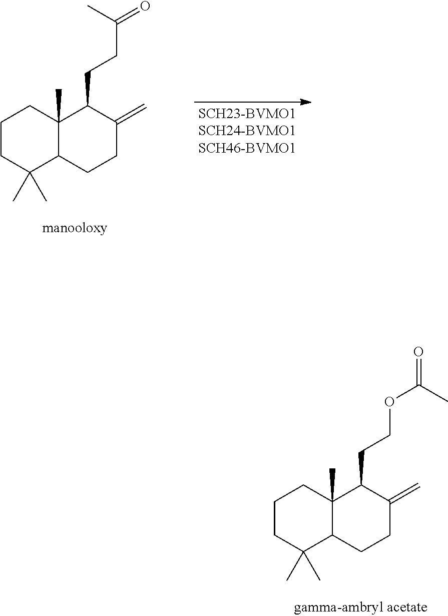 [SMILES: C=C1CCC2C(C)(C)CCC[C@]2(C)[C@H]1CCC(C)=O ; C=C1CCC2C(C)(C)CCC[C@]2(C)[C@H]1CCOC(C)=O]

These results show that SCH23-BVMO1, SCH24-BVMO1 and SCH46-BVMO1 catalyse a Baeyer-Villiger type oxidation of manooloxy.

### Example 2: In-Vivo Conversion of Copalal to Compound 4 Using BVMOs

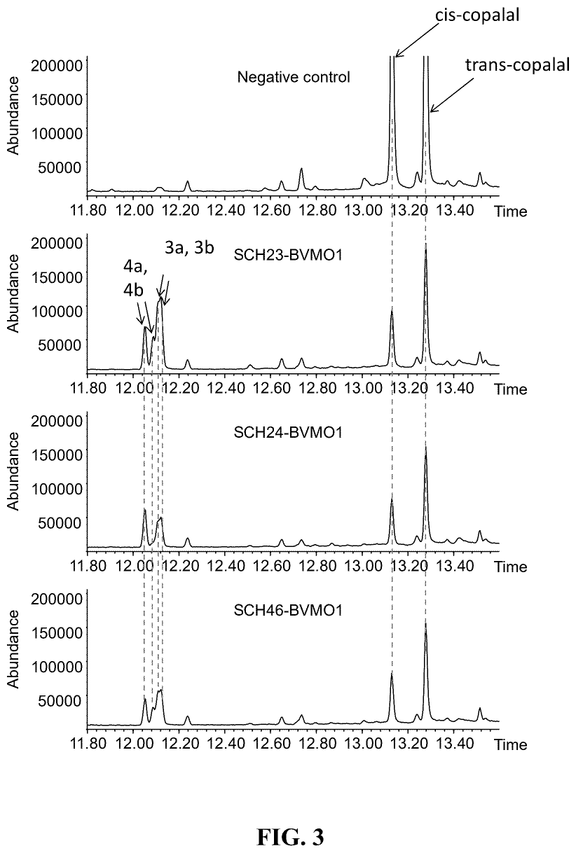

Codon optimized cDNAs encoding for SCH23-BVMO1 from Hyphozyma roseonigra (SEQ ID NO: 3), SCH24-BVMO1 from Filobasidium magnum (SEQ ID NO: 7) and SCH46-BVMO1 from Bensingtonia ciliata (SEQ ID NO: 14) were synthesized and cloned in the pJ414 expression plasmid (ATUM, Newark, California) providing the plasmids pJ414-SCH23-BVMO1, pJ414-SCH24-BVMO1 and pJ414-SCH46-BVMO1. KRX E. coli cells (Promega Corporation, Madison, WI, USA) were transformed with these expression plasmids. The cells were grown and used in whole cell bioconversion assay as described above using a mixture of cis-copalal and trans-copalal as substrate. A negative control was included consisting of the cells transformed with an empty plasmid. In the presence of the SCH23-BVMO1, SCH24-BVMO1 or SCH46-BVMO1 recombinant proteins, conversion of cis-copalal and trans-copalal was observed. The GC-MS analysis of the products (FIG. 3) of the bioconversion after 42 hours of incubation shows the formation of four major products, the two stereoisomers 3a and 3b and the two stereoisomers 4a and 4b.

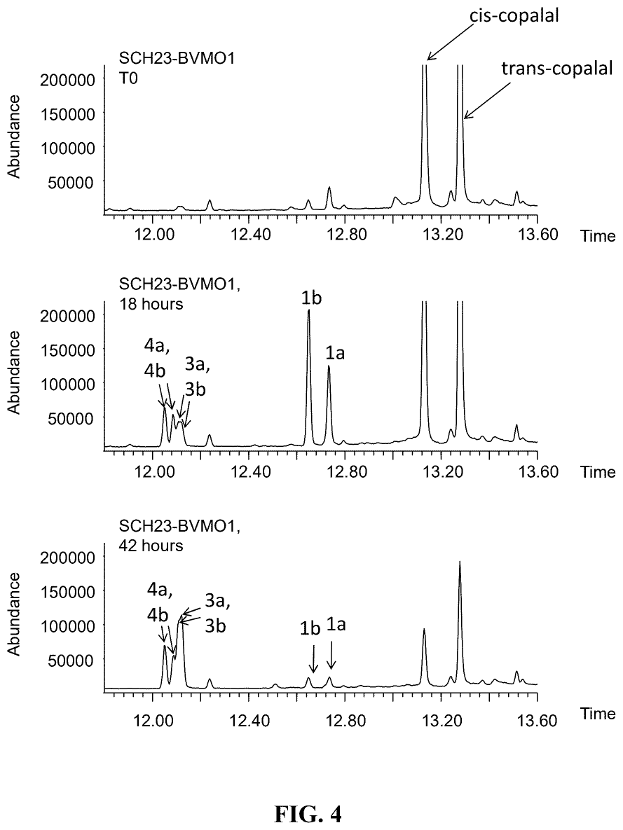

Time point measurements of the bioconversion show the formation of compounds 1a and 1b as intermediate products. FIG. 4 compares GC-MS analysis of the conversion of cis-copalal and trans-copalal by SCH23-BVMO1 at different times; similar evolution of the product profiles is observed with SCH24-BVMO1 and SCH46-BVMO1. The sequential formation of these compounds shows that trans-copalal and cis-copalal are converted to compound 4a and 4b in several steps. Compounds 1a and 1b and compounds 4a and 4b are formate esters. Such functional groups can be formed from aldehyde compounds by Baeyer-Villiger monooxygenases. Thus, the following reaction scheme, involving enzymatic and non-enzymatic (chemical reactions), can be drawn to describe the conversion of trans-copalal by SCH23-BVMO1, SCH24-BVMO1 or SCH46-BVMO1.

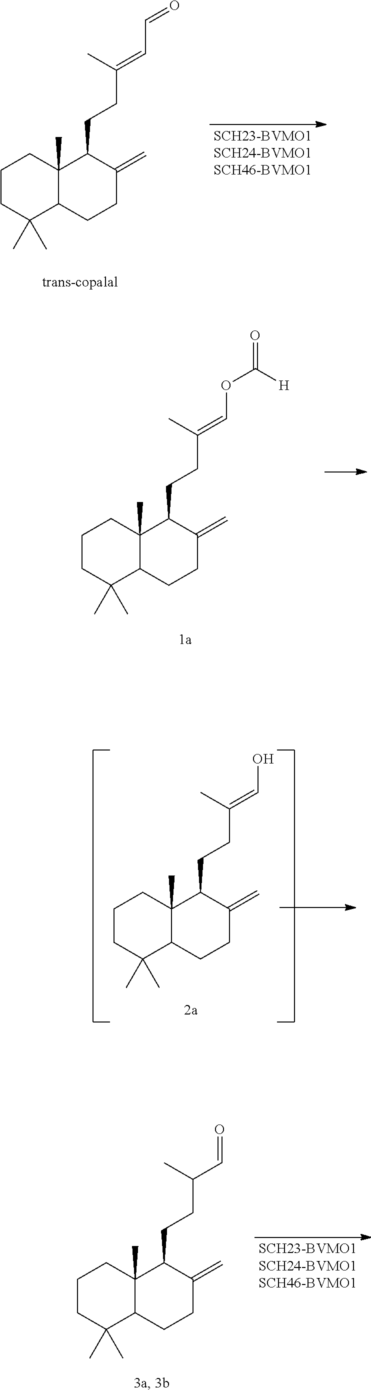

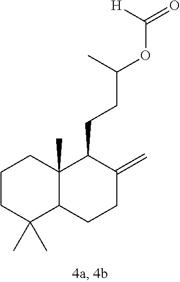 [SMILES: C=C1CCC2C(C)(C)CCC[C@]2(C)[C@H]1CCC(C)OC=O]

In this scheme, the recombinant enzymes catalyse two Baeyer-Villiger type oxidations on two different aldehydes. First, the α,β-unsaturated aldehyde group of trans-copalal is oxidized to form compound 1a in the first Baeyer-Villiger oxidations by the recombinant enzyme. The enol formate functional group of compounds 1a is unstable under the experimental conditions and is partially hydrolysed to form compound 2a. This latter compound is rapidly converted via a keto-enol tautomerization to compound 3 (3a and 3b) and is therefore not detected in the GC-MS analysis. Compound 3 (3a and 3b) is the substrate of the same enzyme which catalyses a second Baeyer-Villiger oxidations to form compound 4 (4a and 4b).

The reaction scheme bellow depicts the similar reactions in the transformation of cis-copalal by SCH23-BVMO1, SCH24-BVMO1 or SCH46-BVMO1.

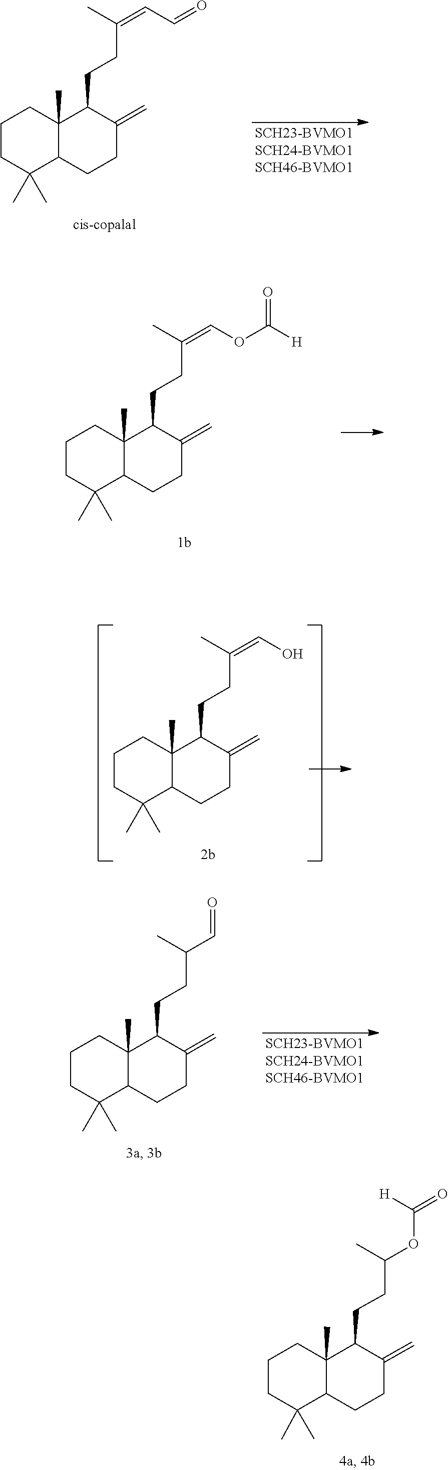

These results show that SCH23-BVMO1, SCH24-BVMO1 and SCH46-BVMO1 catalyse a Baeyer-Villiger type oxidation on labdane aldehyde compounds.

### Example 3: In Vitro Conversion of Manooloxy Using BVMOs and Esterases

For this experiment the following recombinant proteins were used: SCH23-BVMO1 from Hyphozyma roseonigra (SEQ ID NO: 2) SCH24-BVMO1 from Filobasidium magnum (SEQ ID NO: 6), SCH23-EST from Hyphozyma roseonigra (SEQ ID NO: 20) and SCH24-EST from Filobasidium magnum (SEQ ID NO: 24). Codon optimized cDNAs encoding for SCH23-BVMO1 (SEQ ID NO: 3) and SCH24-BVM01 (SEQ ID NO: 7) were synthesized and cloned in the pJ414 expression plasmid (ATUM, Newark, California) providing the plasmids pJ414-SCH23-BVMO1 and pJ414-SCH24-BVMO1. Codon optimized cDNAs encoding for SCH23-EST (SEQ ID NO: 21) and SCH24-EST (SEQ ID NO: 25) were synthesized and cloned in the pJ431 expression plasmid (ATUM, Newark, California) providing the plasmids pJ414-SCH23-EST, pJ414-SCH24-EST.

KRX E. coli cells (Promega Corporation, Madison, WI, USA) were transformed with each of these expression plasmids. The transformed cells were grown and cell free lysates were prepared as described. In vitro enzymatic assays were performed with either of these protein fractions or with a combination of two of these protein fractions. The in vitro assays conditions were as described above with addition of 160 mg/L of manooloxy, 60 UM flavine adenine dinucleotide (FAD) and 500 μM reduced β-Nicotinamide adenine dinucleotide phosphate (NADPH).

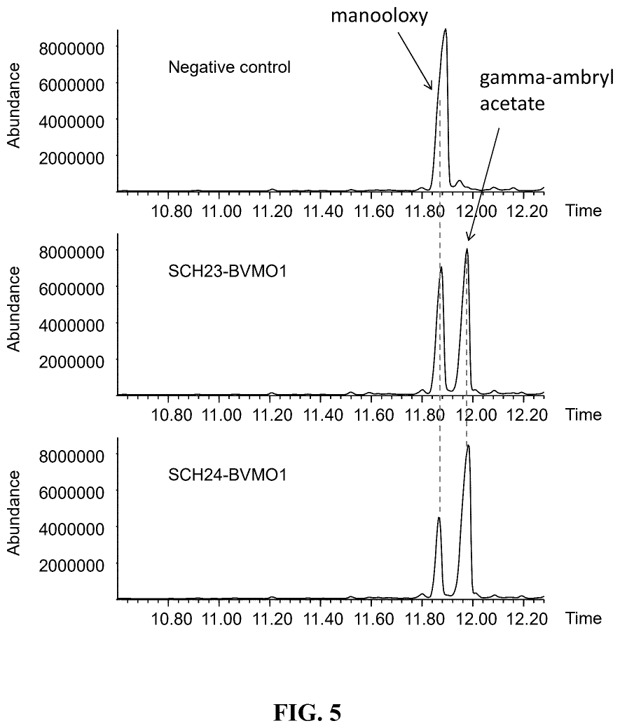

Using crude fractions containing the recombinant SCH23-BVMO1 and SCH24-BVMO1 proteins, conversion of manooloxy to gamma-ambrol acetate was observed. No conversion was detected when using a control lysate obtained from E. coli cells transformed with an empty plasmid (FIG. 5). From these experiments, the following enzymatic reaction can be drawn:

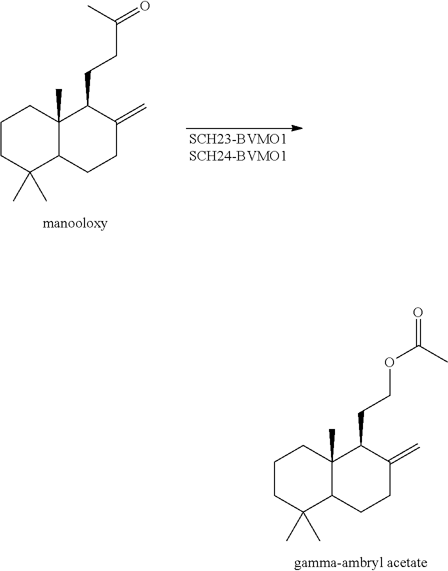 [SMILES: C=C1CCC2C(C)(C)CCC[C@]2(C)[C@H]1CCC(C)=O ; C=C1CCC2C(C)(C)CCC[C@]2(C)[C@H]1CCOC(C)=O]

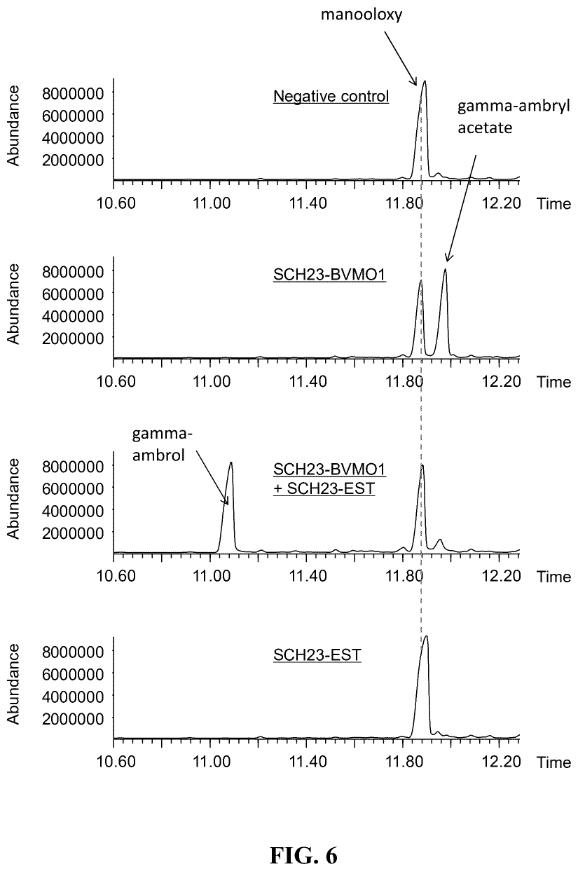

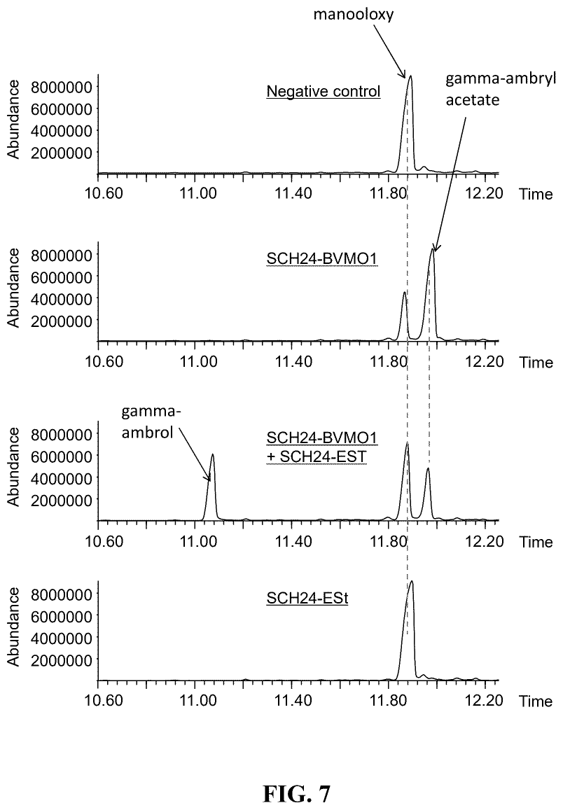

In vitro enzymatic assays were also performed using protein fractions containing a recombinant esterase enzyme and using a combination of protein fractions containing a recombinant BVMO and a recombinant esterase enzyme. These assays were performed as described above using manooloxy as substrate. The GC-MS analysis of the products formed (FIGS. 6 and 7) shows conversion of manooloxy to gamma-ambryl acetate in the presence of a BVMO enzymes (SCH23-BVMO1 or SCH24-BVMO1) and further conversion of gamma-ambryl acetate to gamma-ambrol when an esterase enzyme (SCH23-EST or SCH24-EST) is present in the assay. When the esterase is used in the absence of a BVMO no substrate conversion is observed (FIGS. 6 and 7).

This experiment shows that in the presence of a BVMO and esterase, manooloxy can be converted to gamma-ambrol following the reaction scheme depicted bellow:

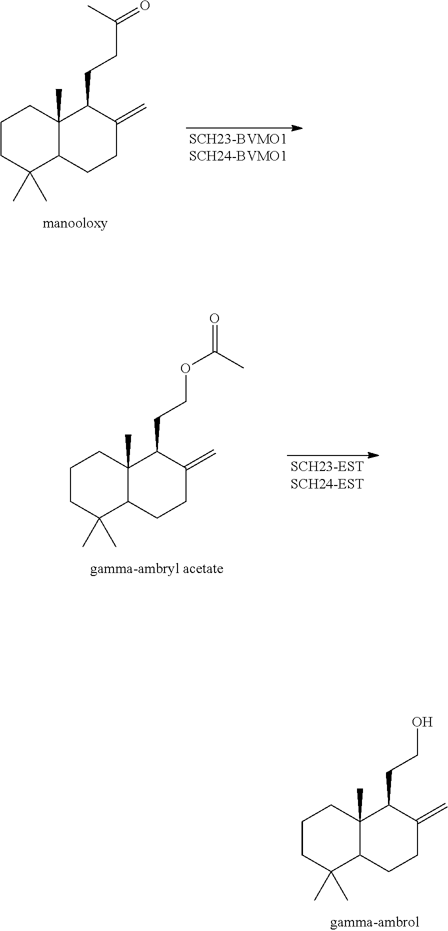 [SMILES: C=C1CCC2C(C)(C)CCC[C@]2(C)[C@H]1CCC(C)=O ; C=C1CCC2C(C)(C)CCC[C@]2(C)[C@H]1CCOC(C)=O ; C=C1CCC2C(C)(C)CCC[C@]2(C)[C@H]1CCO]

### Example 4: In Vitro Conversion of Compounds 4a and 4b to Compounds 5a and 5b Using Esterases

Codon optimized cDNAs encoding for SCH23-EST from Hyphozyma roseonigra (SEQ ID NO: 21), SCH24-EST from Filobasidium magnum (SEQ ID NO: 25) and SCH46-EST from Bensingtonia ciliata (SEQ ID NO: 32) were synthesized and cloned in the pJ414 expression plasmid (ATUM, Newark, California) providing the plasmids pJ414-SCH23-EST1, pJ414-SCH24-EST1 and pJ414-SCH46-EST1. KRX E. coli cells (Promega Corporation, Madison, WI, USA) were transformed with these expression plasmids. The transformed cells were grown and cell free lysates were prepared as described. In vitro enzymatic assays were performed with these protein fractions following the conditions described above.

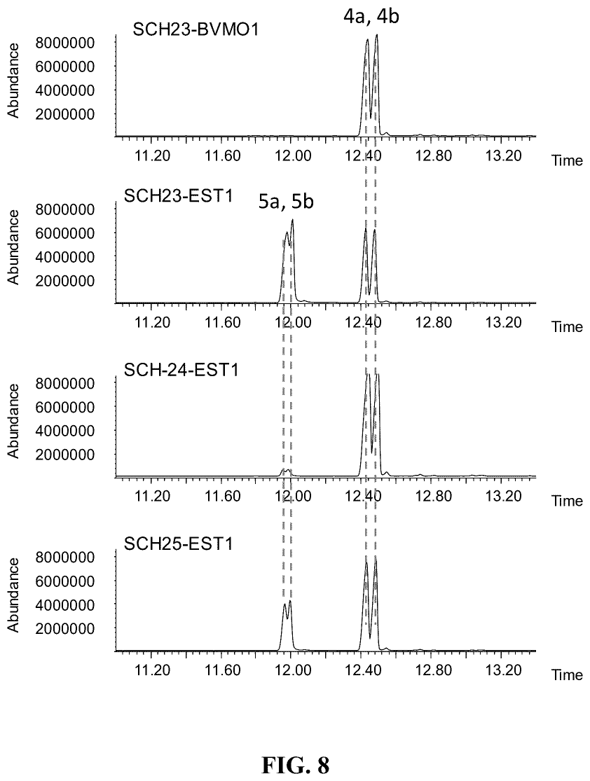

As shown in FIG. 8, using crude fractions containing the recombinant SCH23-EST1, SCH24-EST1 and SCH25-EST1 proteins, the conversions of the two stereoisomers 4a and 4b to compounds 5a and 5b were observed. In comparison, no conversion was detected when using a lysate containing a recombinant BVMO enzyme (these proteins were thus used for the control reactions in this experiment series). Under these conditions the enzymatic activities of SCH23-EST1 and SCH25-EST1 were higher than the enzymatic activity of SCH24-EST.

From these experiments, the following enzymatic reaction can be drawn:

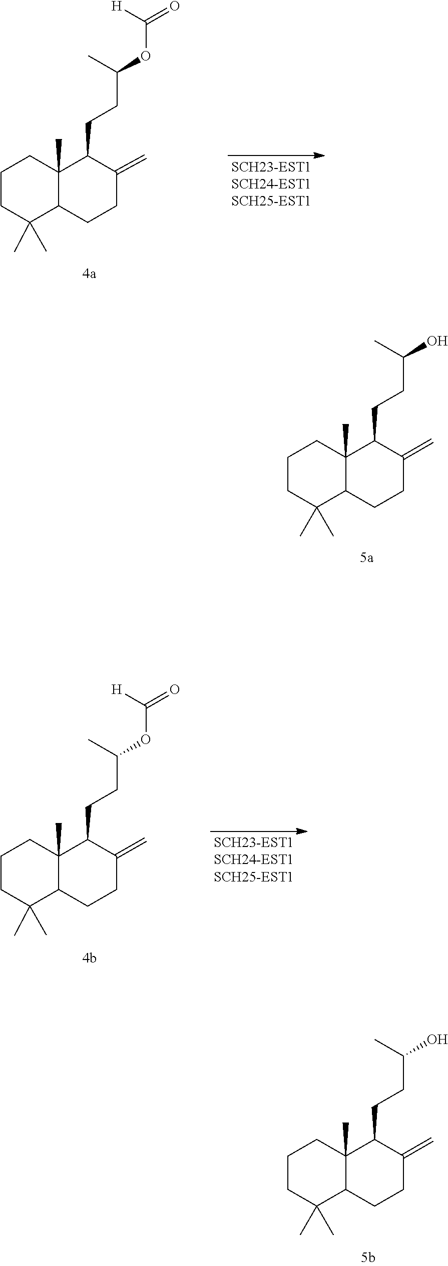 [SMILES: C=C1CCC2C(C)(C)CCC[C@]2(C)[C@H]1CC[C@@H](C)OC=O ; C=C1CCC2C(C)(C)CCC[C@]2(C)[C@H]1CC[C@@H](C)O ; C=C1CCC2C(C)(C)CCC[C@]2(C)[C@H]1CC[C@H](C)OC=O ; C=C1CCC2C(C)(C)CCC[C@]2(C)[C@H]1CC[C@H](C)O]

### Example 5: In Vitro Conversion of Copalal Using BVMOs and Esterases

For this experiment the following recombinant proteins were used: SCH23-BVMO1 from Hyphozyma roseonigra (SEQ ID NO: 2), SCH24-BVMO1 from Filobasidium magnum (SEQ ID NO: 6), SCH25-BVMO1 from Papiliotrema laurentii (SEQ ID NO: 10), SCH23-EST from Hyphozyma roseonigra (SEQ ID NO: 20), SCH24-EST from Filobasidium magnum (SEQ ID NO: 24), SCH25-EST from Papiliotrema laurentii (SEQ ID NO: 28).

Codon optimized cDNAs encoding for SCH23-BVMO1 (SEQ ID NO: 3), SCH24-BVMO1 (SEQ ID NO: 7) and SCH25-BVMO1 (SEQ ID NO: 11) were synthesized and cloned in the pJ414 expression plasmid (ATUM, Newark, California) providing the plasmids pJ414-SCH23-BVMO1, pJ414-SCH24-BVMO1 and pJ414-SCH25-BVMO1. Codon optimized cDNAs encoding for SCH23-EST (SEQ ID NO: 21), SCH24-EST (SEQ ID NO: 25) and SCH25-EST (SEQ ID NO: 29) were synthesized and cloned in the pJ431 expression plasmid (ATUM, Newark, California) providing the plasmids pJ414-SCH23-EST, pJ414-SCH25-EST.

KRX E. coli cells (Promega Corporation, Madison, WI, USA) were transformed with these expression plasmids. The transformed cells were grown and cell free lysates were prepared as described. In vitro enzymatic assays were performed with protein fractions containing a recombinant BVMO enzyme or a recombinant esterase enzyme or by combining of protein fractions containing recombinant BVMO and esterase enzymes. The assays were performed as described above with addition of 320 mg/L of a mixture of cis-copalal and trans-copalal as substrate, 60 UM flavine adénine dinucleotide (FAD) and 500 μM reduced β-Nicotinamide adenine dinucleotide phosphate (NADPH).

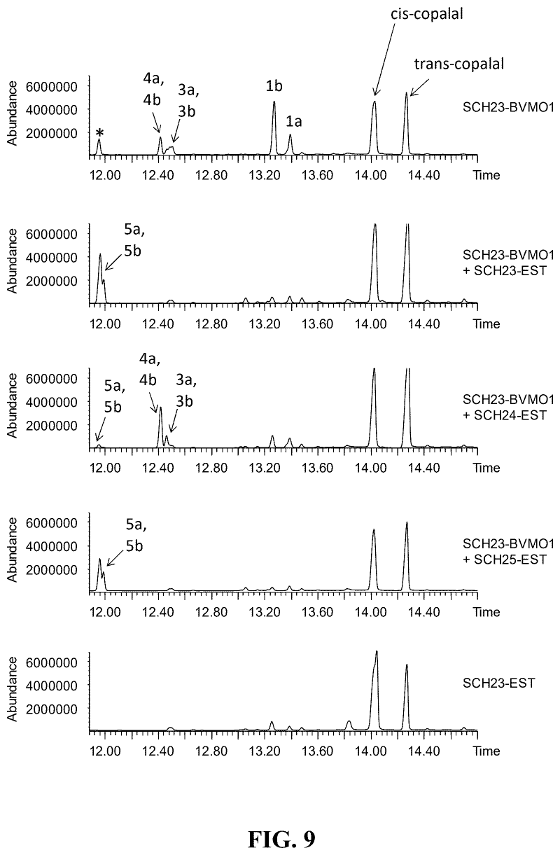

FIG. 9 compares the products of the conversion of copalal in the presence of SCH23-BVMO1 only and in combination with different esterase enzymes. In the presence of SCH23-BVMO1, the major products are the formate compounds 1a, 1b and 4a, 4b. When the assays were conducted in the additional presence of the SCH23-EST or SCH25-EST, the major products of the conversion were compounds 5a and 5b showing that these two esterase enzymes can efficiently hydrolyse the formate intermediates produced by the BVMO enzyme. In the additional presence of SCH24-EST, the hydrolysis of the same intermediates (1a, 1b, and 4a, 4b) was observed, however with this enzyme the hydrolysis of the intermediates 1a and 1b by SCH24-EST seems more efficient than the hydrolysis of the intermediates 4a and 4b.

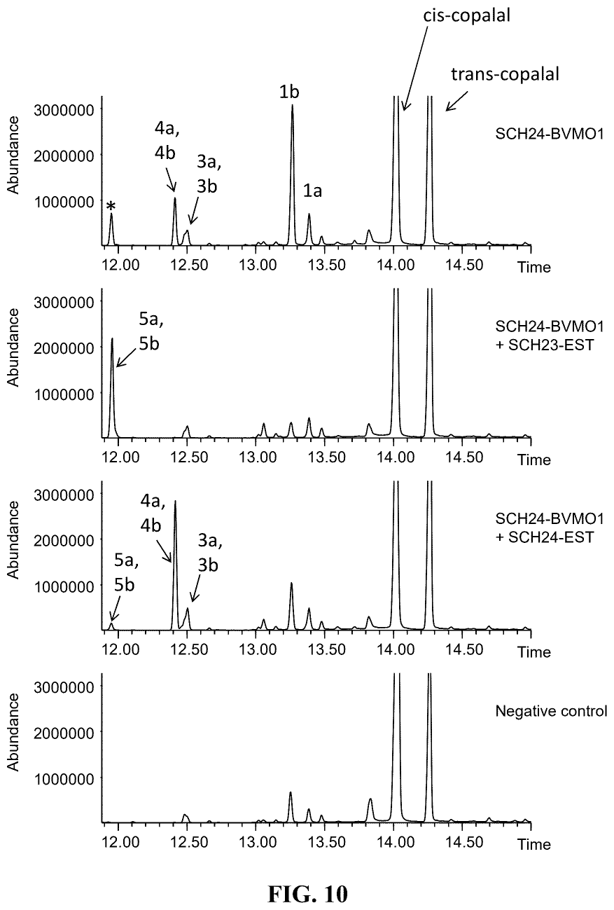

Similar conversion of cis- and trans-copalal was observed when SCH24-BVMO1 was combined with esterase SCH23-EST or SCH24-EST (FIG. 10). In control experiments, when copalal was incubated only with an esterase, no conversion was observed.

From these experiments the following enzyme pathway can be deduced.

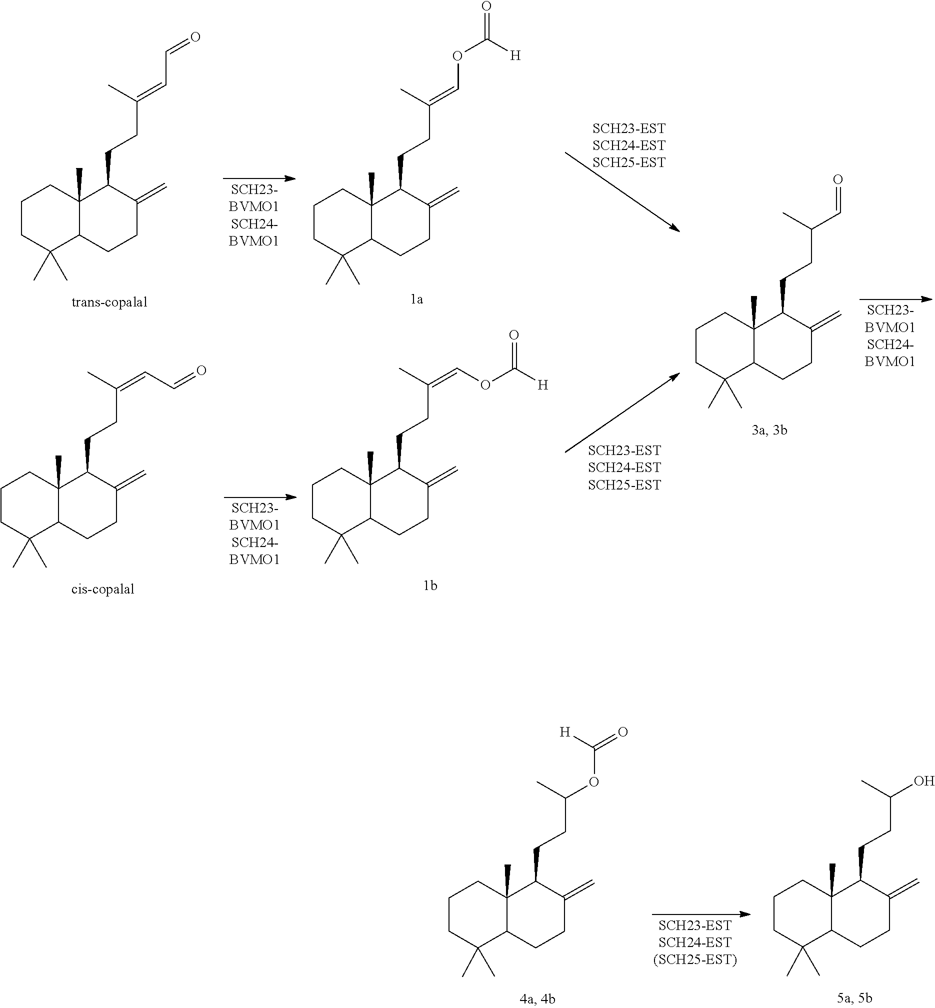

### Example 6: In-Vivo Production of the 14,15-Dinor-Labdane Compounds 5a and 5b and Biosynthetic Intermediates in Engineered Bacteria Cells Expressing a BVMO and an Esterase

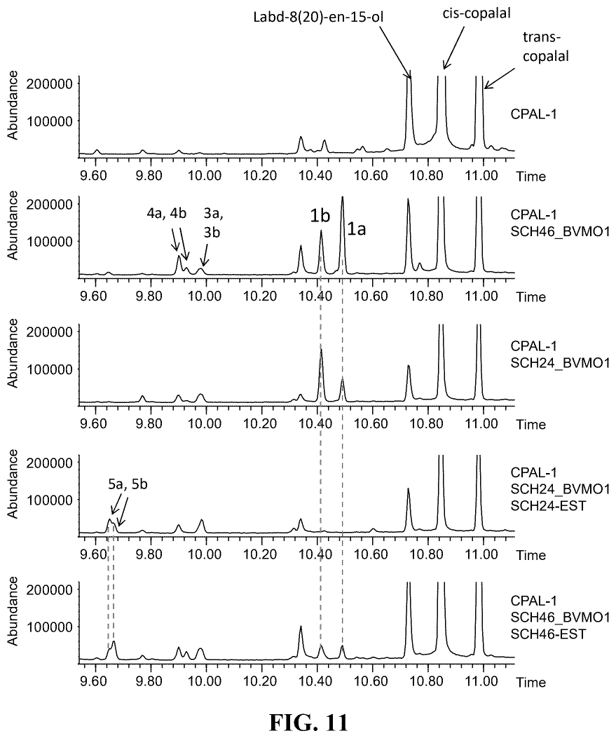

In this experiment, the plasmid pJ401-CPAL-1 (described above) was used to transform E. coli cells to produce copalal as described in the experimental section. When DP1205 E. coli cells were transformed and cultivated unter the conditions described in the experimental section, formation of trans-copalal and cis-copalal was observed (FIG. 11, upper chromatogram). The detection of the two double-bond isomers of copalal is due to the relative easy isomerization of (E)-α,β-unsaturated aldehydes (Konning et al. Org. Lett., 2012, 14 (20), pp 5258-5261). The additional detection of labd-8 (20)-en-15-ol is due to E. coli endogenous enoate reductase activity.

The bacteria cells were then transformed with a second expression plasmid carrying a codon optimized cDNA encoding for SCH24-BVMO1 from Filobasidium magnum (ATCC® 20918™) (SEQ ID NO: 7) or SCH46-BVMO1 from Bensingtonia ciliata (SEQ ID NO: 14). These plasmid was prepared by cloning the optimized cDNAs in the pJ423 expression plasmid (ATUM. Newark. California) providing the plasmids pJ423-SCH23-BVMO and pJ423-SCH46-BVMO, respectively. The cells transformed with two plasmids were cultivated and the production of terpene compounds and terpene derivatives was analysed using the conditions described in the experimental section. Under these conditions the compounds 1a and 1b. 3a and 3b, and 4a and 4b were detected in the solvent extract of the culture broth (FIG. 11). These results show that, using these combinations of enzymes, the biosynthesis of a labdane diterpene such as copalol and the sequential enzymatic cleavage of two carbon-carbon bounds in the side chain can be introduced in a recombinant cell.

Similarly, bacteria cells were co-transformed with the plasmid pJ401-CPAL-1 and with a second plasmid carrying a gene encoding for a BVMO and a gene encoding for an esterase. pJ423-SCH24-BVMO-SCH24-EST, prepared by inserting a synthetic operon composed of a codon optimized cDNA encoding SCH24-BVMO1 (SEQ ID NO: 7) and a codon optimized cDNA encoding SCH24-EST (SEQ ID NO: 25) into the pJ423 expression plasmid (ATUM. Newark. California), or pJ423-SCH46-BVMO-SCH46-EST, a plasmid prepared by inserting a synthetic operon composed of a codon optimized cDNA encoding SCH46-BVMO (SEQ ID NO: 14) and a codon optimized cDNA encoding SCH46-EST (SEQ ID NO: 32) into the pJ423 expression plasmid (ATUM. Newark. California). The cells were cultivated and the production of terpene compounds and terpene derivatives was analysed using the conditions described in the experimental section. Under these conditions, the compounds 5a and 5b were detected and decreased amounts of the pathway intermediates (compounds 1a, 1b, 3a, 3b, 4a and 4b) were observed.

This experiment series shows that the following biosynthetic pathway can be introduced in a host cells transformed to express diterpene biosynthesis enzymes in combination with a BVMO and an esterase.

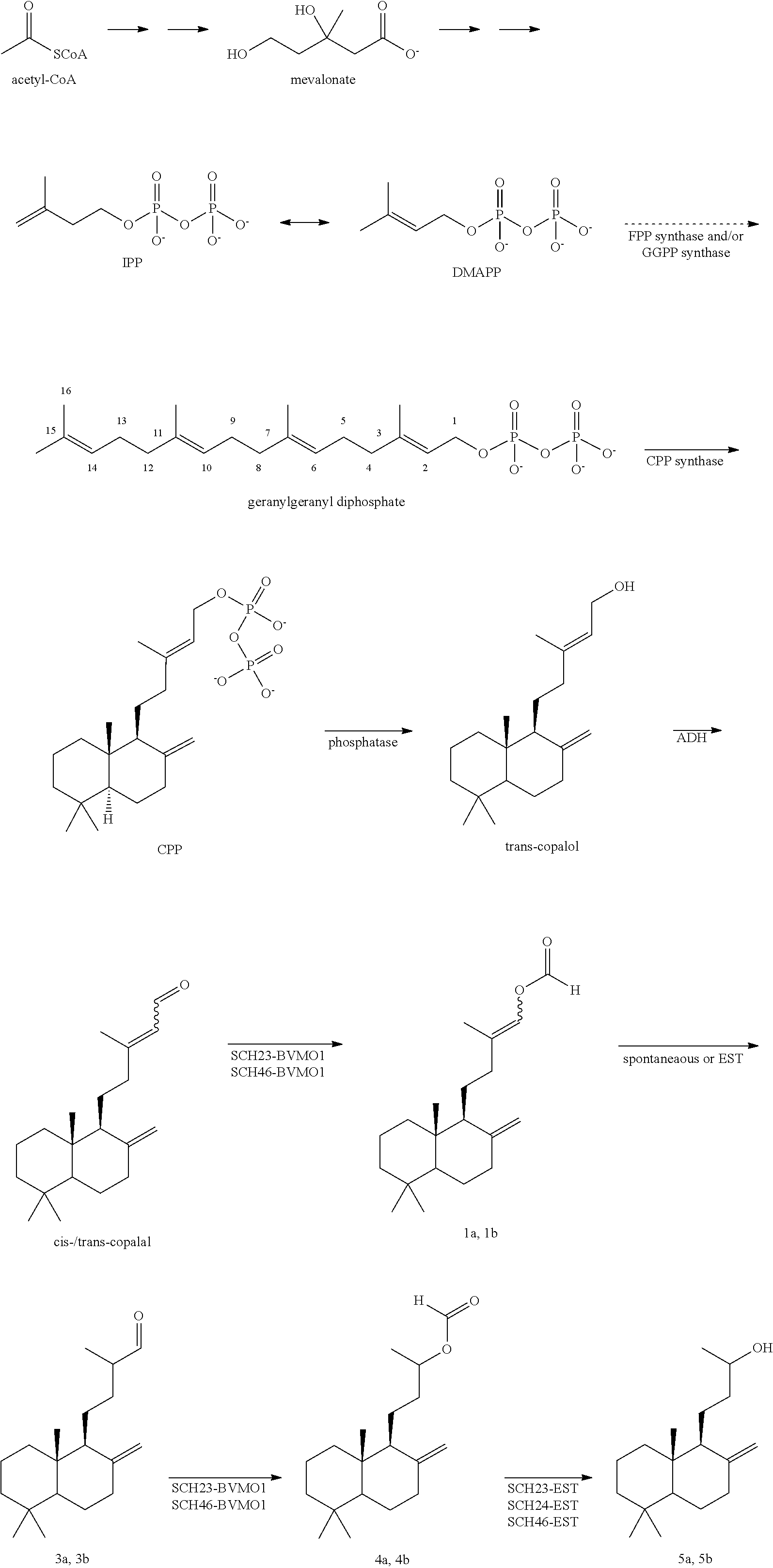

### Example 7: In-Vivo Conversion of Compounds 5a and 5b to Manooloxy Using Alcohol Dehydrogenases

For this experiment, the following alcohol dehydrogenases were evaluated for the oxidation of compounds 5a and 5b to manooloxy:
RrhSecADH from Rhodococcus rhodochrous (SEQ ID NO: 146),
SCH80-00043 from Rhodococcus rhodochrous (SEQ ID NO: 149),
SCH80-04254 from Rhodococcus rhodochrous (SEQ ID NO: 152),
SCH80-06135 from Rhodococcus rhodochrous (SEQ ID NO: 155),
SCH80-06582 from Rhodococcus rhodochrous (SEQ ID NO: 158),
(see also WO2005/026338); the above ADHs are merely non-limiting examples and may be replaced by other known ADHs may

Codon optimized cDNAs encoding for each of these proteins were synthesized and cloned in the vector pJ401 providing plasmids pJ401-RrhSecADH, pJ401-SCH80-00043, pJ401-SCH80-04254, pJ401-SCH80-06135 and pJ401-SCH80-06582 (ATUM, Newark, California).

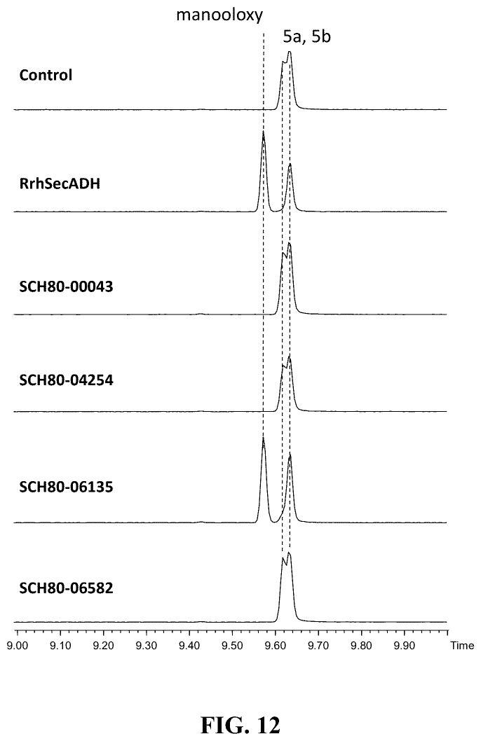

KRX E. coli cells (Promega Corporation, Madison, WI, USA) were transformed with these expression plasmids. The transformed cells were grown and used in a whole cell bioconversion assay as described above using a mixture of compounds 5a and 5b as substrate. Five hours after the induction of the expression of the recombinant proteins, the substrate was added to a final concentration of 0.55 mg/mL using an emulsion containing 50 mg/mL of tween 80 and 25 mg/mL of substrate in water. A negative control was included consisting of the cells transformed with an empty plasmid. The oxidation reaction was observed only in the presence of the SCH80-06135 and RrhSecADH recombinant proteins (FIG. 12) showing that these enzymes can catalyse the following reaction.

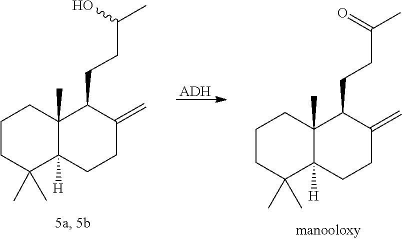

### Example 8: In-Vivo Production of the Tetranor-Labdane Compounds Gamma-Ambrol and Biosynthetic Intermediates in Engineered Bacteria Cells Expressing a BVMO, an Esterase and an Alcohol Dehydrogenase

In this experiment, the plasmid pJ401-CPAL-1 (described above) was used to transform the DP 1205 E. coli cells creating a background strain producing copalal (cis- and trans-isomer) as described in the previous section.

This strain was then co-transformed with the plasmid pJ423-SCH24-BVMO-SCH24-EST (described above) allowing a further expression of a BVMO and an esterase in the same cells. In accordance with the observation made in the previous section, this recombinant organism produces 14,15-dinor-labdane compounds.

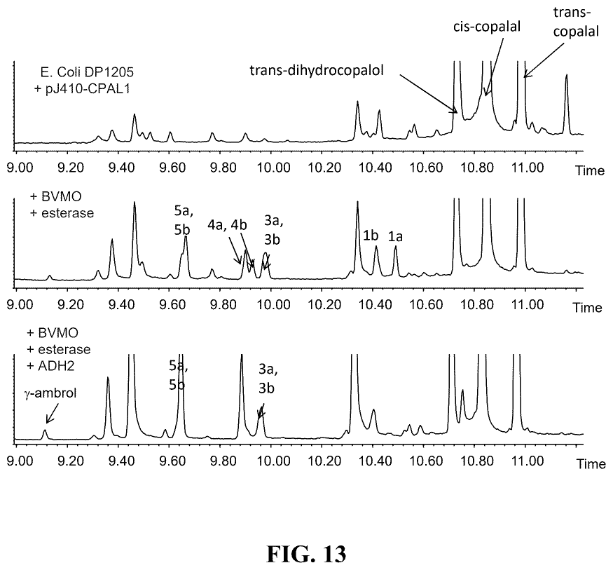

To allow the side-chain degradation to continue to the formation of tetranor-labdane derivatives, the secondary alcohol group of compounds 5a and 5b must be oxidized to the corresponding ketone. A plasmid was thus constructed containing nucleotide sequences encoding for a BVMO, an esterase and an appropriate alcohol dehydrogenase (identified in Example 7). For the alcohol dehydrogenase, a codon optimized cDNA encoding for RrhSecADH from a Rhodococcus species (Accession number WP_043801412.1) (SEQ ID NO: 147) was synthesised and a synthetic operon was designed combining the RrhSecADH cDNA and the cDNAs encoding for SCH24-BVMO and SCH24-EST. The operon was cloned into the pJ423 expression plasmid providing the pJ423-secADH-23BVMO-EST plasmid. When DP1205 E. coli cells co-transformed with the vector pJ401-CPAL-1 and the vector pJ423-secADH-23BVMO-EST were cultivated under the conditions described above, gamma-ambrol was detected in the GC-MS analysis of the cultivation broth (FIG. 13). These data show that when compound 5 (5a and 5b) is oxidized to manooloxy in the presence of an appropriate ADH, the BVMO can catalyse the following step in the pathway providing gamma-ambrol.

This experiment series shows that the following biosynthetic pathway can be introduced in a recombinant host cells.

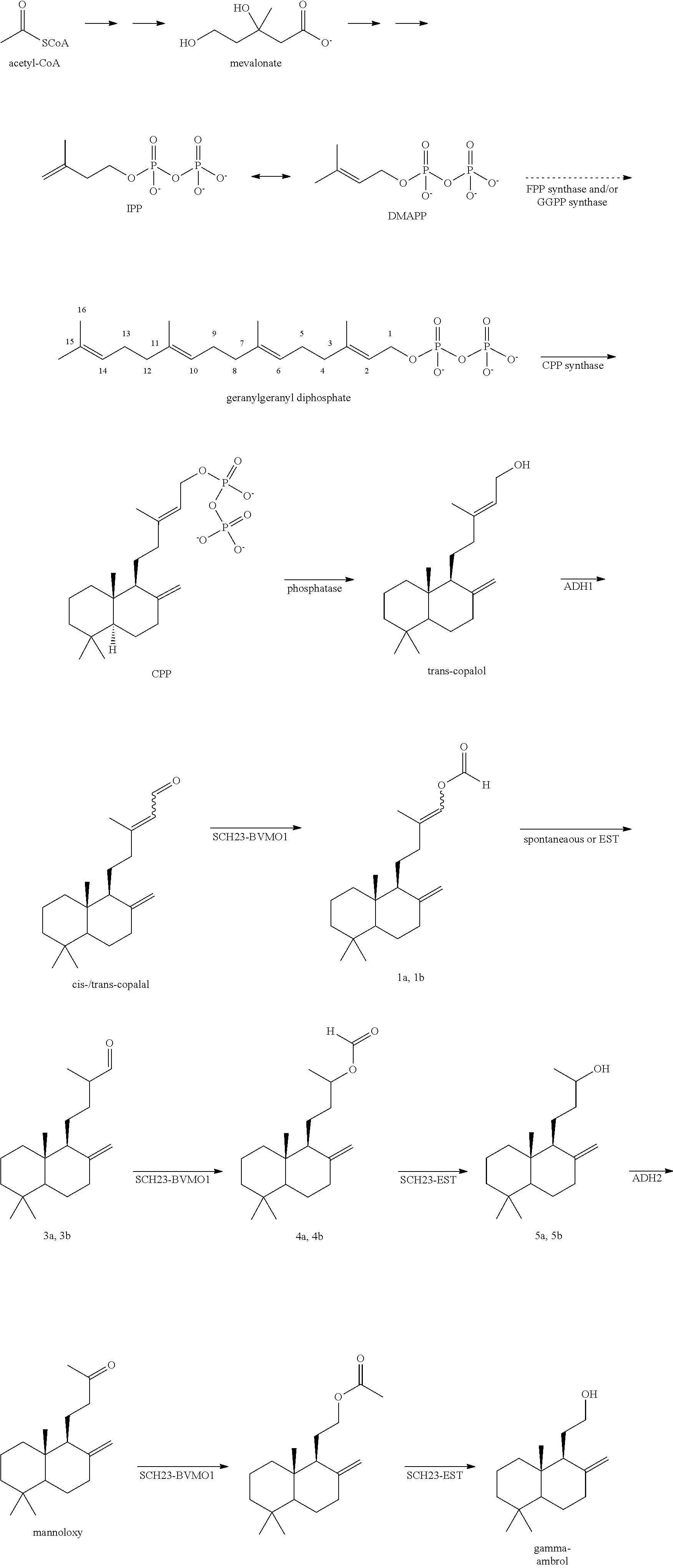

### Example 9: In Vivo Manooloxy Production in Saccharomyces cerevisiae Cells Using Alcohol Dehydrogenases (ADHs), Baeyer-Villiger Monooxygenases (BVMOs) and Esterases (ESTs) from Hyphozyma roseonigra or Cryptococcus albidus

For the production of manooloxy, the genes encoding for the GGPP synthase carG (from Blakeslea trispora, NCBI accession JQ289995.1) (SEQ ID NOs: 182), the copalyl-pyrophosphate synthase SmCPS2 (from Salvia miltiorrhiza, NCBI accession ABV57835.1) (SEQ ID NOs: 185), the copalyl-pyrophosphate phosphatase TalVeTPP (from Talaromyces verruculosus, NCBI accession KUL89334.1) (SEQ ID NOs: 194) and either the alcohol dehydrogenase SCH23-ADH1 (SEQ ID NOs: 134), the Baeyer-Villiger monooxygenase SCH23-BVMO1 (SEQ ID NOs: 2), the esterase SCH23-EST (SEQ ID NOs: 20) and the alcohol dehydrogenase SCH23-ADH2 (from Hyphozyma roseonigra) (SEQ ID NOs: 137) or the alcohol dehydrogenase SCH24-ADH1 (SEQ ID NOs: 140), the Baeyer-Villiger monooxygenase SCH24-BVMO1 (SEQ ID NOs: 6), the esterase SCH24-EST1 (SEQ ID NOS: 24) and the alcohol dehydrogenase SCH24-ADH2 (from Filobasidium magnum) (SEQ ID NOs: 143) were expressed in the engineered Saccharomyces cerevisiae strain YST075 as described in the general methods section above. All genes were codon optimized for their expression in S. cerevisiae (SCH23-ADH1, SEQ ID NO: 135; SCH23-BVMO1, SEQ ID NO: 4; SCH23-EST, SEQ ID NO: 22; SCH23-ADH2, SEQ ID NO: 138; SCH24-ADH1, SEQ ID NO: 141; SCH24-BVMO1, SEQ ID NO: 8; SCH24-EST, SEQ ID NO: 26; SCH24-ADH2, SEQ ID NO: 144; carG, SEQ ID NO: 183; SmCPS2, SEQ ID NO: 186; and TalVeTPP. SEQ ID NO: 195).

The strains YST120 (with SCH23-ADH1, SCH23-BVMO1, SCH23-EST and SCH23-ADH2) and YST121 (with SCH24-ADH1a, SCH24-BVMO1, SCH24-EST and SCH24-ADH2) harboring also the plasmid system for copalol biosynthesis were obtained and cultivated under the conditions described in the general methods section above.

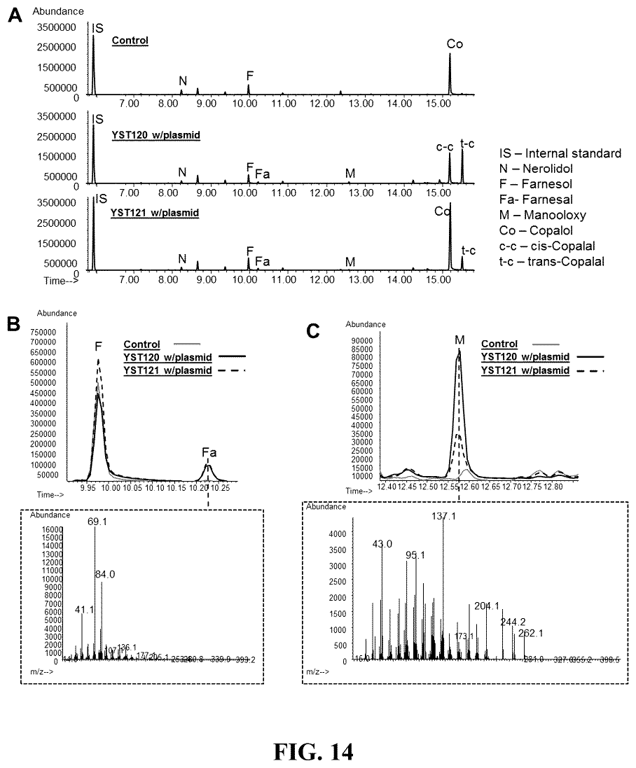

Under these conditions, copalol was identified in all cultures. Only strains containing SCH23-ADH1 or SCH24-ADH1 were able to convert copalol into copalal (FIG. 14A). In addition, farnesal was detected in the cultures where the alcohol dehydrogenases were expressed (FIG. 14B). Accumulation of nerolidol and farnesol was identified in all cultures (FIG. 14A).

In addition, manooloxy was identified in the cultures containing the strains YST120 and YST121 harboring the plasmid with copalol biosynthetic genes (FIG. 14C). Neither gamma-ambryl acetate nor gamma-ambrol was identified. However, the presence of manooloxy suggests that the BVMOs, ESTs and ADHs were functionally expressed in the engineered yeast cells. We hypothesize that the amount obtained of manooloxy was limiting for the BVMOs to catalyze the conversion to gamma-ambryl acetate.

### Example 10: In Vivo Manooloxy Production in Saccharomyces cerevisiae Cells Using Alcohol Dehydrogenases (ADHs), Baeyer-Villiger Monooxygenases (BVMOs) and Esterases (ESTs) from Hyphozyma roseonigra or Cryptococcus albidus

For the production of manooloxy, the genes encoding for the GGPP synthase carG (from Blakeslea trispora, NCBI accession JQ289995.1), the copalyl-pyrophosphate synthase SmCPS2 (from Salvia miltiorrhiza, NCBI accession ABV57835.1), the copalyl-pyrophosphate phosphatase TalVeTPP (from Talaromyces verruculosus, NCBI accession KUL89334.1), the alcohol dehydrogenase SCH23-ADH1 and either the Baeyer-Villiger monooxygenase SCH23-BVMO1 and the esterase SCH23-EST (from Hyphozyma roseonigra) or the Baeyer-Villiger monooxygenase SCH24-BVMO1 and the esterase SCH24-EST (from Cryptococcus albidus) were expressed in the engineered Saccharomyces cerevisiae strain YST075 as described in the general methods section.

The obtained strains were termed YST177 (with carG, SmCPS2, TalVeTPP, SCH23-ADH1, SCH23-BVMO1 and SCH23-EST) and YST178 (with carG, SmCPS2, TalVeTPP, SCH23-ADH1, SCH24-BVMO1 and SCH24-EST) and were cultivated as described in the general methods section above. Cultures were analyzed by GC-MS as described above.

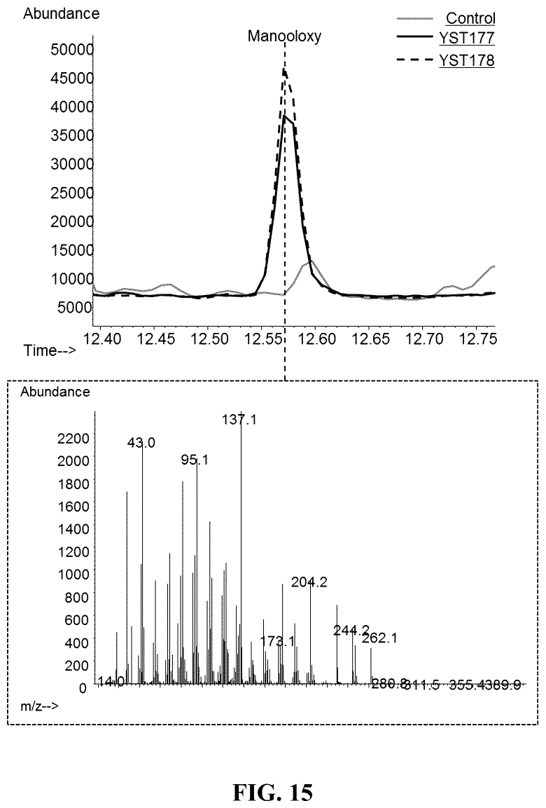

Copalol, copalal, nerolidol, farnesol and farnesal were identified in the cultures after extraction. The engineered cells not containing the alcohol dehydrogenases SCH23-ADH2 or SCH24-ADH2 were expected to accumulate the intermediate 5a (or 5b) and to be incapable to produce manooloxy. Interestingly, manooloxy was identified (FIG. 15) and molecule 5a (or 5b) was not detected. These results suggest that SCH23-ADH2 and SCH24-ADH2 might contribute to the production of manooloxy in yeast cells but are not essential for its production under the conditions tested. We hypothesize that endogenous alcohol dehydrogenase activities in yeast are responsible for the conversion.

### Example 11: Characterisation of a SCH94-3944, an Enzyme from Rhodococcus erytheropolis with Carbon-Carbon Bond Cleavage Activity

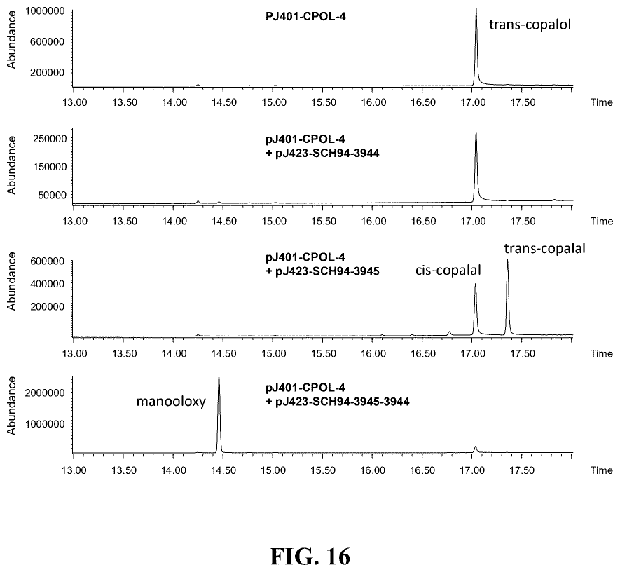

In this experiment, the plasmid pJ401-CPOL-4 (described above) was used to transform the DP1205 E. coli cells creating a background strain producing copalol. The transformed strain produced copalol as major product with a concentration of up to 500 mg/L in the culture media in the tube assay (FIG. 16).

This strain was then further transformed with a second plasmid carrying one or more E. coli codon optimized cDNAs derived from R. erytheropolis. Two cDNAs were selected:
SCH94-3945, encoding for a putative alcohol dehydrogenase (SEQ ID NO: 161),
SCH94-3944, encoding for a 157 amino acid protein containing two protein family domains: a “GXWXG” protein domain (pfam14231, pfam.xfam.org/) and a domain of unknown function “DUF4334” (pfam14232, pfam.xfam.org/) (SEQ ID NO: 34).

Expression vectors were prepared using pJ423 as background and containing either a codon optimized cDNA encoding for SCH94-3945 (pJ423-SCH94-3945) or SCH94-3944 (pJ423-SCH94-3944) or a bicistronic operon comprised of the optimized cDNAs encoding for SCH94-3945 and SCH94-3944 (pJ423-SCH94-3944-3945).

When cells were transformed with the vector pJ401-CPOL-4 and the vector pJ423-SCH94-3944, no difference was observed in comparison with cells transformed with pJ401-CPOL-4 only, showing that the SCH94-3944 recombinant protein does not transform copalol. When cells were transformed with the vector pJ401-CPOL-4 and the vector pJ423-SCH94-3945, formation of cis-copalal and trans-copalal was observed showing that the SCH94-3945 is an alcohol dehydrogenase able to oxidase copalol to copalal (FIG. 16).

When cells were transformed with the vector pJ401-CPOL-4 and the vector pJ423-SCH94-3944-3945, formation of manooloxy was observed as major product with a concentration of up to 1

g/L in the culture media in the tube assay. Under this assay condition, the conversion of cis- and trans-copalal was nearly complete (FIG. 16).

This experiment shows that the SCH94-3944 enzyme can cleave the alpha-beta carbon-carbon double-bound of copalal and catalyse the direct conversion of cis-copalal and trans-copalal to the 14,15-dinor-labdane compound manooloxy, as shown in the scheme below.

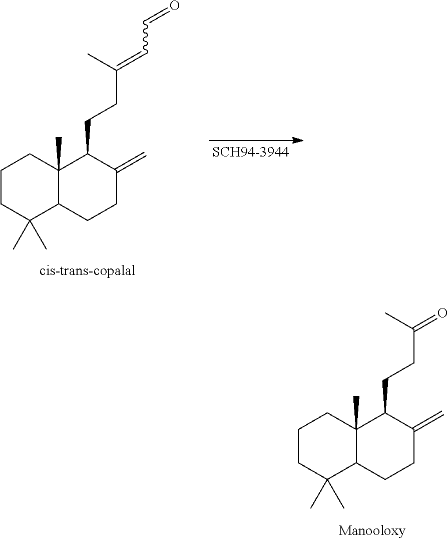 [SMILES: C=C1CCC2C(C)(C)CCC[C@]2(C)[C@H]1CCC(C)=CC=O ; C=C1CCC2C(C)(C)CCC[C@]2(C)[C@H]1CCC(C)=O]

### Example 12: In-Vivo Conversion of Cis- and Trans-Farnesal Using an Enal-Cleaving Polypeptide from Rhodococcus erythropolis

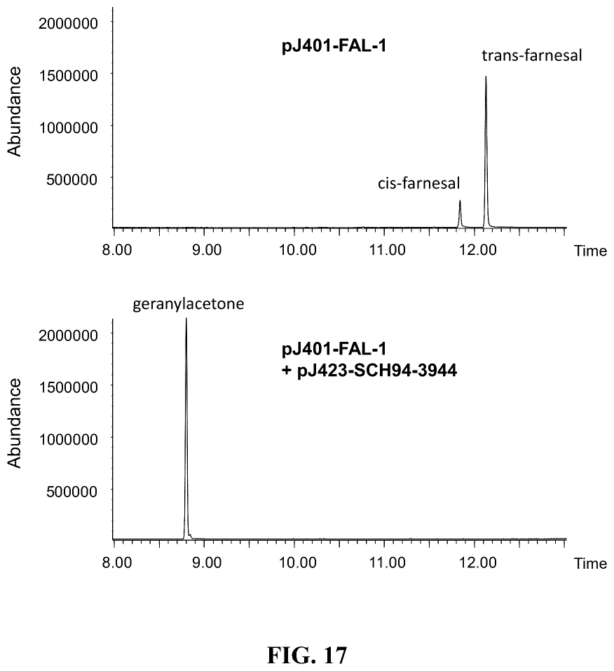

In this experiment, the plasmid pJ401-FAL-1 (described above) was used to transform the DP1205 E. coli cells creating a background strain producing cis-farnesal and trans-farnesal as major products with a concentration up to 500 mg/L in the culture media in tube assay conditions (FIG. 17).

This strain was then further transformed with the plasmid pJ423-SCH94-3944 carrying a cDNA encoding for SCH94-3944 from R. erytheropolis. The GC-MS analysis of the compounds produced by the cells showed formation of geranylacetone (FIG. 17). This experiment thus shows that the SCH94-3944 enzyme can cleave the alpha-beta carbon-carbon double-bound of the acyclic compound farnesal and catalyse the direct conversion of cis-farnesal and trans-farnesal to geranylacetone as shown in the scheme below.

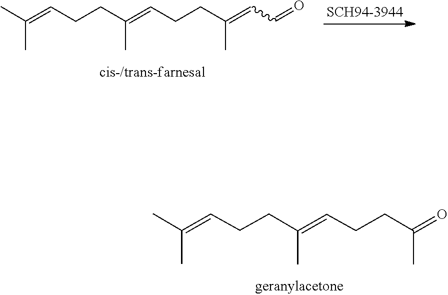 [SMILES: CC(C)=CCC/C(C)=C/CCC(C)=CC=O ; CC(=O)CC/C=C(\C)CCC=C(C)C]

No conversion with farnesol was observed un ed the applied test conditions.

### Example 13. In-Vivo Conversion of Citral Using an Enal-Cleaving Polypeptide from Rhodococcus erythropolis

Biochemical conversion of compounds was performed using E. coli KRX (Promega) cells transformed with the plasmid pJ423-SCH94-3944, thus, overexpressing the SCH94-3944 recombinant protein. The substrate was added to the cell culture to a final concentration of 12 g/L using an 2:1 substrate:Tween 80 emulsion. The bioconversion was performed as described in the experimental section. Negative controls were performed using cells transformed with a pJ423 expression plasmid without insert. Several substrates were tested: citral (a mixture composed of geranial and neral), citronelal (2,3-dihydrocitral) and (E)-2-dodecanal. The cells were incubated for 24 hours in the presence of the various compounds and the products of the conversion were analysed as described in the experimental section.

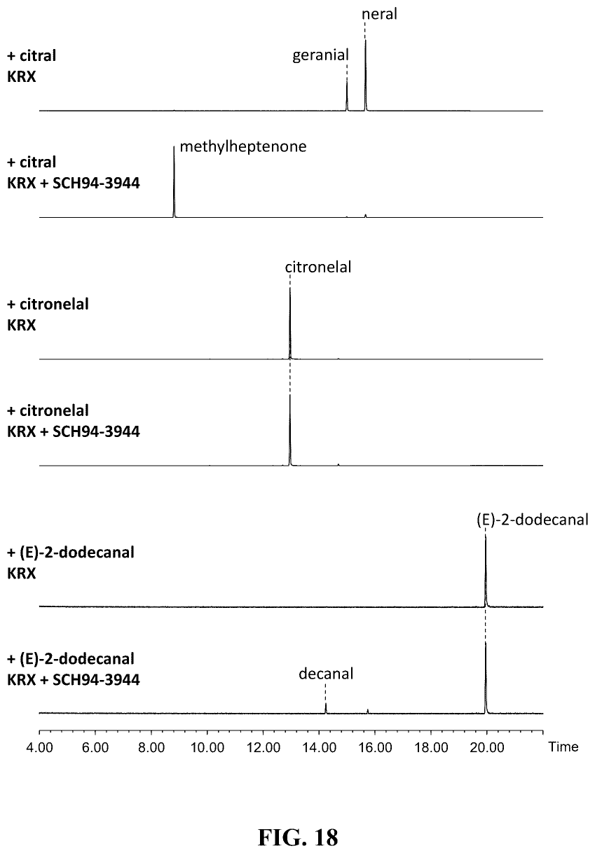

In the presence of the SCH94-3944 recombinant protein, geranial and neral were both converted to methylheptenone (FIG. 18) showing that this enzyme can cleave alpha-beta carbon-carbon double-bound of the acyclic monoterpene aldehydes as shown in the scheme below.

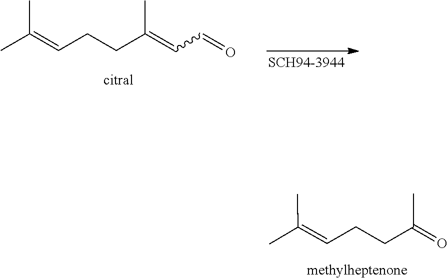

No conversion was obtained with citronelal of the formula

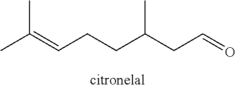

in the presence of the SCH94-3944 recombinant protein (FIG. 18), showing that the unsaturation of α,β-carbon bond is required for the catalysis.

With (E)-2-dodecanal,

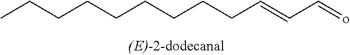

conversion to decanal was observed. However, compared to citral, the conversion yield was significantly lower (FIG. 18). This observation suggests that the absence of the 3-methyl group has a negative effect on the enzymatic conversion by the SCH94-3944 protein.

### Example 14: In Vivo Conversion of Copalal and Farnesal Using GXWXG and DUF4334 Domain Containing Proteins from Other Organisms

The SCH94-3944 protein sequence contains a GXWXG protein family domain and a DUF4334 protein family domain. Proteins with similar domain architectures were searched in other organisms and tested to determine if the enzymatic activity associated with SCH94-3944 can also be associated with these homologous enzymes.

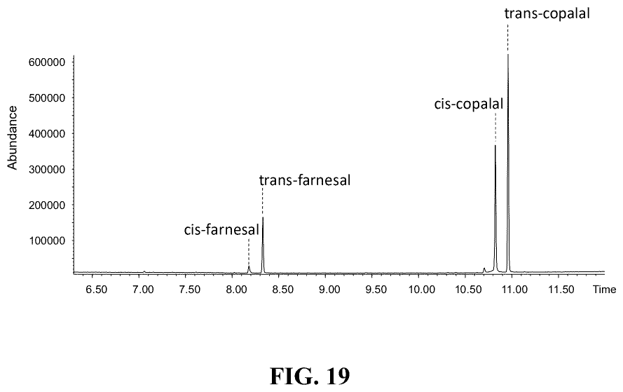

In this experiment, the plasmid pJ401-CPAL-1 (described above) was used to transform the DP1205 E. coli cells creating a background strain producing copalal (cis- and trans-isomer) as described in the previous section. In this strains a FPP synthase is expressed from the genomic integrated operons. Because the terpenyl phosphatase AspWeTPP can dephosphorylate FPP in addition to GGPP, and because AzeTolADH1 can also oxidize farnesol, a significant amount of trans farnesal was detected in addition to copalal when the pJ401-CPAL-1 was used to transforme the DP1205 cells (FIG. 19).

This strain was then co-transformed with a second plasmid carrying a gene encoding for a protein containing a GXWXG protein family domain and a DUF4334 protein family domain. Several proteins were selected:
SCH80-05241 from Rhodococcus rhodochrous (RATCC 12674™) (SEQ ID NO: 38),
Pdigit7033 from Penicillium digitatum (SEQ ID NO: 42),
PitalDUF3443-1 from Penicillium italicum (SEQ ID NO: 46),
AspWeDUF3443 from Aspergillus wentii (SEQ ID NO: 49),
RhoagDUF4334-2 from Rhodococcus hoagii strain PAM2288 (SEQ ID NO: 53),
RhoagDUF4334-3 from Rhodococcus hoagii strain N128 (SEQ ID NO: 56),
RhoagDUF4334-4 from Rhodococcus hoagii NBRC 10125 (SEQ ID NO: 59),
CnecaDUF4334 from Cupriavidus necator (SEQ ID NO: 62),
Rins-DUF4334 from Ralstonia insidiosa (SEQ ID NO: 69),
CgatDUF4334 from Cryptococcus gattii EJB2 (SEQ ID NO: 72),
GclavDUF4334 from Grosmannia clavigera kw1407 (SEQ ID NO: 75),
TcurvaDUF4334 from Thermomonospora curvata (SEQ ID NO: 81), and
PprotDUF4334 from Pseudomonas protegens (SEQ ID NO: 87).

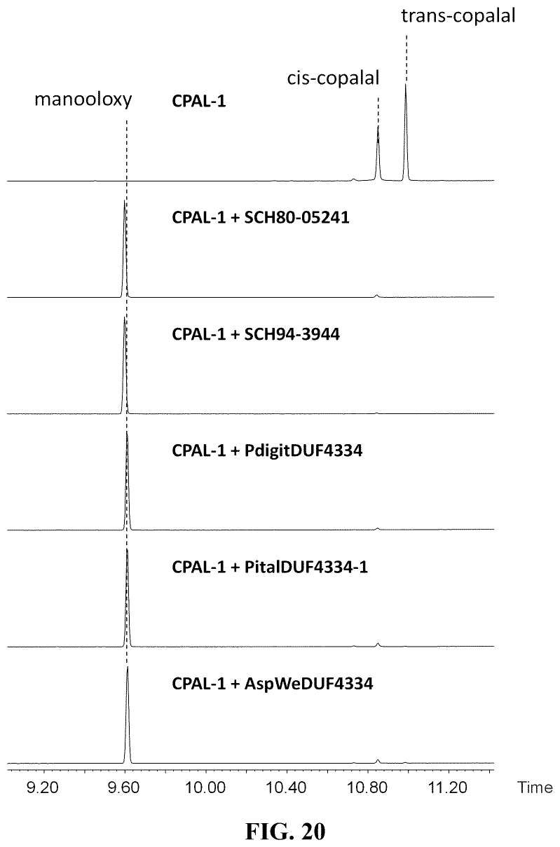

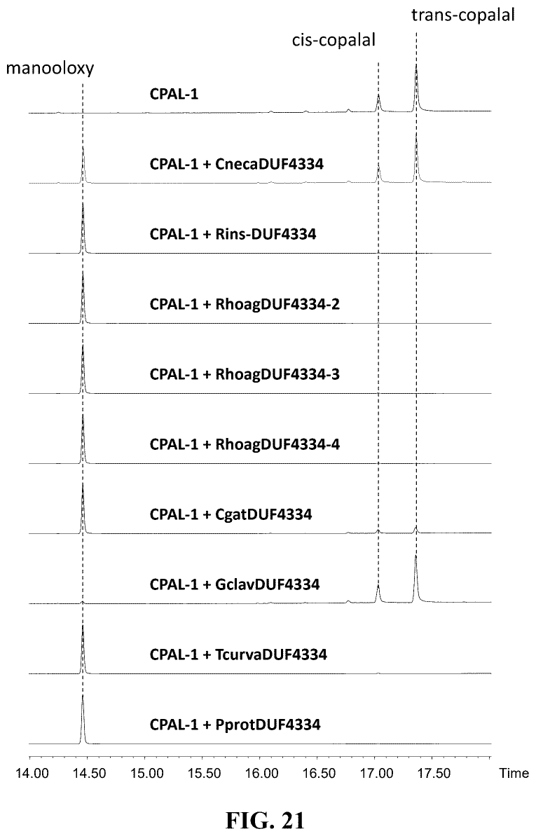

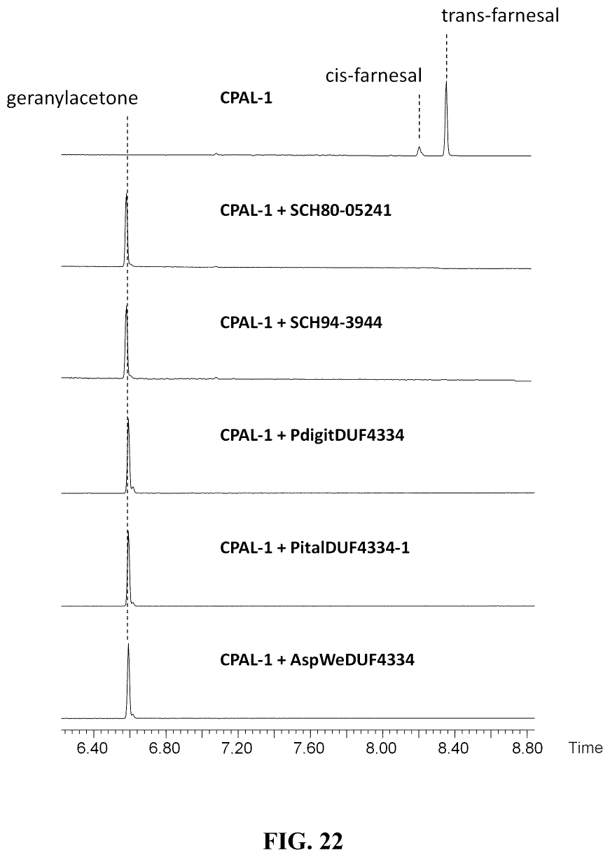

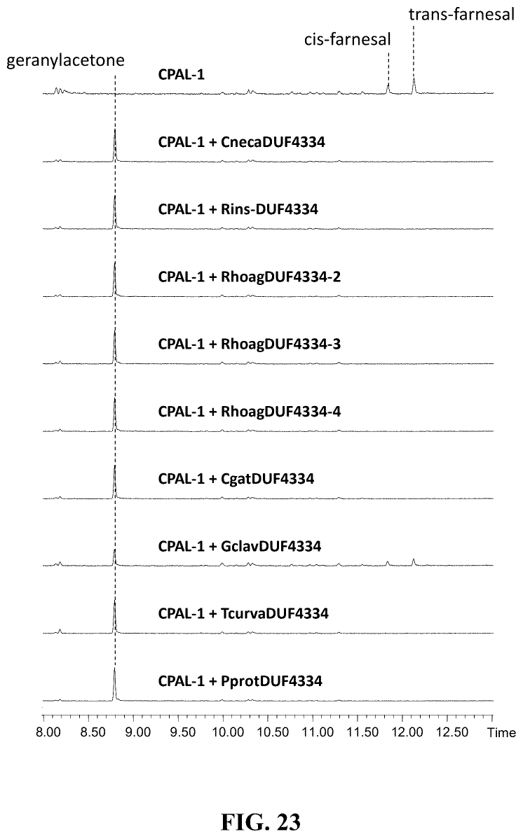

Codon optimized cDNAs encoding for each of these proteins were designed and cloned in the pJ423 expression plasmids (ATUM, Newark, California). The DP1205 E. coli cells were co-transformed with one of these plasmids and with the pasmid pJ401-CPAL-1. FIGS. 20 and 21 show the conversion of cis-copalal and trans-copalal to manooloxy in the presence of each of the recombinant proteins containing a GXWXG and DUF4334 domain. Under the assay conditions the conversion of copalal was almost complete with each recombinant enzyme except for the GclavDUF4334 enzyme with which only a small conversion was observed. FIGS. 22 and 23 show the conversion of cis-farnesal and trans-farnesal to geranylacetone. The conversion of fanesal was also complete with each enzyme except for GclavDUF4334 with which only about 50% of the farnesal was converted.

This experiment shows that proteins containing a GXWXG protein family domain in the N-terminal region and a DUF4334 protein family domain in the C-terminal region can catalyse enal-cleaving activity on copalal and farnesal as shown in the schemes below.

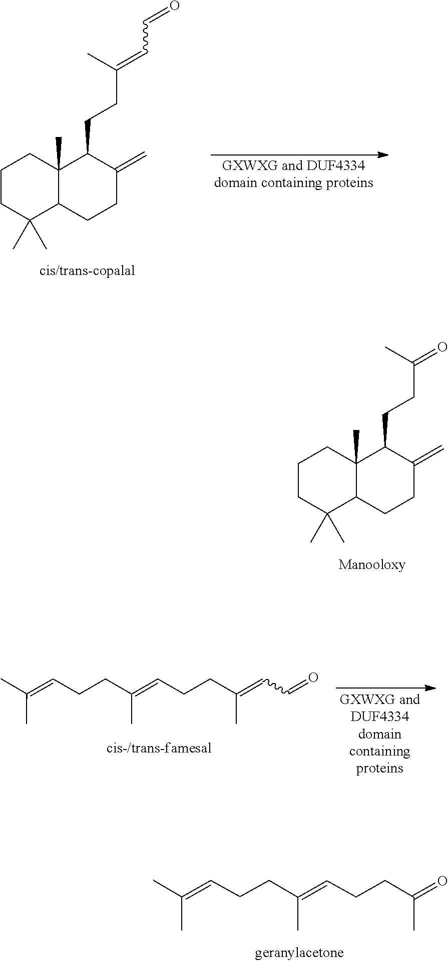 [SMILES: C=C1CCC2C(C)(C)CCC[C@]2(C)[C@H]1CCC(C)=CC=O ; C=C1CCC2C(C)(C)CCC[C@]2(C)[C@H]1CCC(C)=O ; CC(C)=CCC/C(C)=C/CCC(C)=CC=O ; CC(=O)CC/C=C(\C)CCC=C(C)C]

### Example 15: Variants of SCH94-3944 with Single Amino Acid Modification

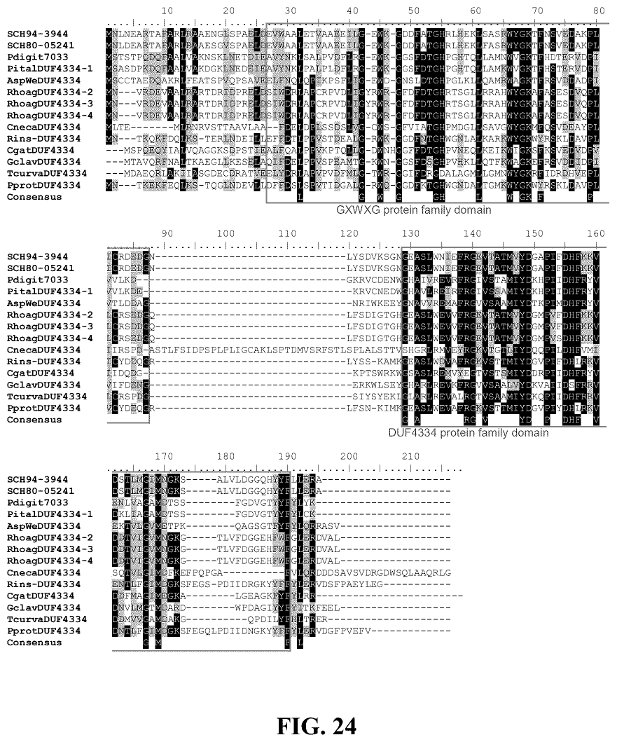

The alignment of the amino acid sequences of the GXWXG and DUF4334 domain containing proteins having enal-cleaving activities, showed conserved amino acids along the amino acid sequence and within said two protein domains (FIG. 24). Conserved residues in protein families are often important for the enzymatic activity.

To evaluate the participation of the conserved residues in the GXWXG and DUF4334 domain containing enzymes to the enzymatic activity, artificial mutants of the SCH94-3944 protein were design in which the conserved residues were individually replaced by an alanine residue. The following residue were mutated: W44, T51, H53, L59, W64, K67, S71, R¹⁰⁶, Y115, D116, D122, M136, K139, F152, L154 and R¹⁵⁶. The modified proteins were designated SCH94-3944-W44A, SCH94-3944-T51A, SCH94-3944-H53A, SCH94-3944-L59A, SCH94-3944-W64A, SCH94-3944-K67A, SCH94-3944-S71A, SCH94-3944-R106A, SCH94-3944-Y115A, SCH94-3944-D116A, SCH94-3944-D122A, SCH94-3944-M136A, SCH94-3944-K139A, SCH94-3944-F152A, SCH94-3944-L154A and SCH94-3944-R156A.

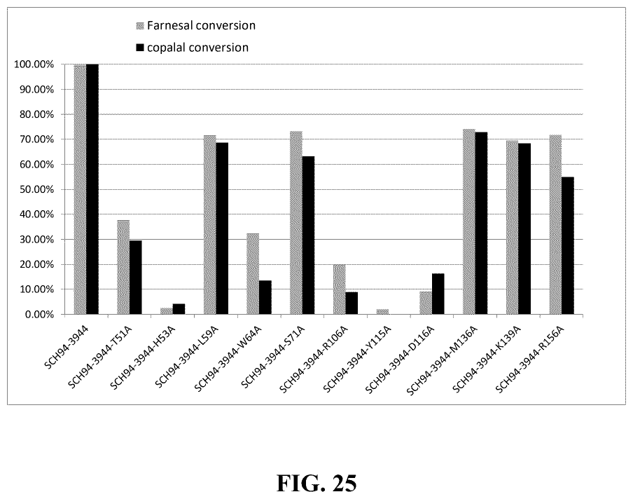

Codon optimized cDNAs encoding for each of these proteins were designed and cloned in the pJ423 expression plasmids (ATUM, Newark, California). The DP1205 E. coli cells were co-transformed with one of these plasmids and with pasmid pJ401-CPAL-1. In the presence of the SCH94-3944-W44A, SCH94-3944-K67A, SCH94-3944-D122A, SCH94-3944-F152A or SCH94-3944-L154A recombinant proteins, no conversion of copalal and farnesal was observed. In the presence of the SCH94-3944-T51A, SCH94-3944-H53A, SCH94-3944-L59A, SCH94-3944-W64A, SCH94-3944-S71, SCH94-3944-R106A, SCH94-3944-Y115A, SCH94-3944-D116A, SCH94-3944-M136A, SCH94-3944-K139A and SCH94-3944-R156A enzymes, conversion of copalal and farnesal was observed but with an efficiency lower than the wild type SCH94-3944 protein. FIG. 25 shows the activity of each single amino acid variants enzyme relative to the wild type SCH94-3944.

### Example 16: In-Vivo Production of γ-Ambryl Acetate by Combining the Enal Cleaving Activity and the BVMO Activity in E. coli Cells

In this experiment, the plasmid pJ401-CPAL-1 (described above) was used to transform the DP1205 E. coli cells creating a background strain producing copalal (cis- and trans-isomer) as described above.

This strain was then co-transformed with a second plasmid carrying a codon optimized nucleotide sequence encoding for either an enzyme with enal-cleaving activity or an enzyme with BVMO activity, or with a second vector carrying an operon composed of a codon optimize cDNA encoding for an enal-cleaving polypeptide and codon optimized cDNA encoding for a BVMO:
pJ423-AspWeBVMO, containing an optimized DNA sequence encoding for AspWeBVMO (SEQ ID NO: 17);
pJ423-SCH94-3944, containing an optimized DNA sequence encoding for SCH94-3944 (SEQ ID NO: 35);
pJ423-SCH94-3944-SCH23-BVMO, containing an optimized DNA sequence encoding for SCH94-3944 and SCH23-BVMO1 (SEQ ID NOs: 35 and 3);
pJ423-SCH94-3944-SCH24-BVMO, containing an optimized DNA sequence encoding for SCH94-3944 and SCH23-BVMO1 (SEQ ID NOs: 35 and 7);
pJ423-SCH94-3944-SCH46-BVMO, containing an optimized DNA sequence encoding for SCH94-3944 and SCH46-BVMO1 (SEQ ID NOs: 35 and 14).

The transformed cells were cultivated and the formation of terpene derivatives was analysed by GC-MS as described above.

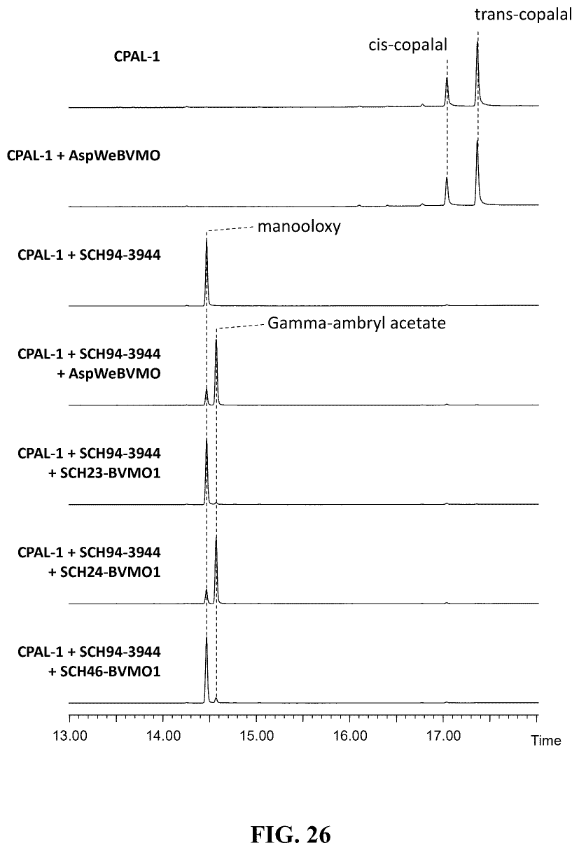

When cells were transformed with the vector pJ401-CPAL-1 and with an empty pJ423 vector or pJ423-AspWeBVMO, formation of only cis-copalal and trans-copalal was observed. (FIG. 26).

When cells were transformed with the vector pJ401-CPAL-1 and with pJ423-SCH94-3944, formation of manooloxy was observed with complete conversion of copalal (FIG. 26). When cells were transformed with the vector pJ401-CPAL-1 and with a pJ423 vector allowing the co-expression of a enal-cleaving polypeptide and a BVMO, formation of γ-ambryl acetate was observed in the addition of manooloxy. Variations in the ratio of manooloxy and gamma-ambryl acetate were observed depending on the BVMO enzyme.

This experiment shows that the following pathway can be introduced in a host cell to produce gamma-ambryl acetate.

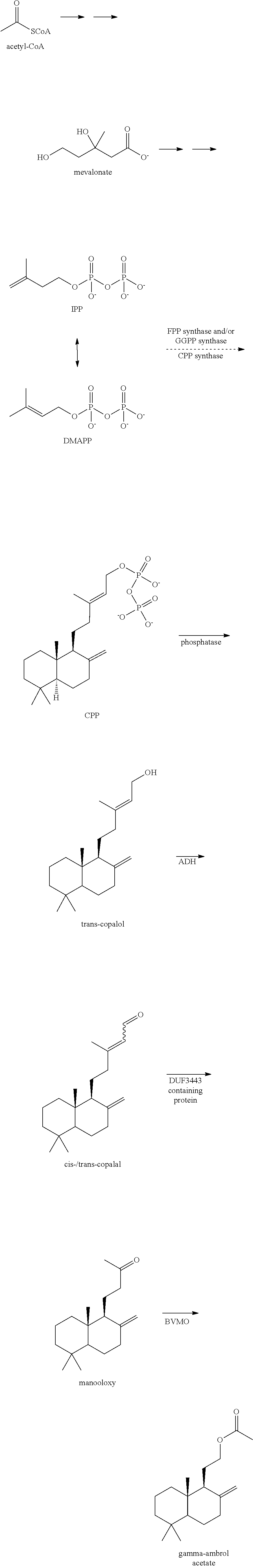

### Example 17: In Vivo Manooloxy Production in Saccharomyces cerevisiae Cells Using SCH23-ADH1 from Hyphozyma roseonigra and Different Enal Cleaving Polypeptides

For the production of manooloxy, the genes encoding for the GGPP synthase carG (from Blakeslea trispora, NCBI accession JQ289995.1), the copalyl-pyrophosphate synthase SmCPS2 (from Salvia miltiorrhiza, NCBI accession ABV57835.1), the copalyl-pyrophosphate phosphatase TalVeTPP (from Talaromyces verruculosus, NCBI accession KUL89334.1), the alcohol dehydrogenase SCH23-ADH1 (from Hyphozyma roseonigra) and one of the tested enal-cleaving polypeptides were expressed in the engineered Saccharomyces cerevisiae strain YST075 as described in the general methods section.

Five enal-cleaving polypeptides were evaluated:
AspWeDUF4334 (from Aspergillus wentii; GenBank accession OJJ34591.1) (SEQ ID NO: 49).
CnecaDUF4334 (from Cupriavidus necator; GenBank accession WP_049800708.1) (SEQ ID NO: 62).
Pdigit7033 (from Penicillium digitatum) (SEQ ID NO: 42).
SCH94-3944 (from Rhodococcus erytheropolis) (SEQ ID NO: 34).
SCH80-05241 (from Rhodococcus rhodochrous).

All genes were codon optimized for their expression in S. cerevisiae (AspWeDUF4334, SEQ ID NO: 51: CnecaDUF4334, SEQ ID NO: 64: Pdigit7033, SEQ ID NO: 44; SCH94-03944, SEQ ID NO: 36; and SCH80-05241 SEQ ID NO: 40).

The constructed strains were termed YST184 (with AspWeDUF4334), YST185 (with CnecaDUF4334), YST186 (with Pdigit7033), YST187 (with SCH94-03944) and YST188 (with SCH80-05241). These strains were cultivated as described in the general methods section above: the production of manooloxy and other compounds was identified using GC-MS analysis.

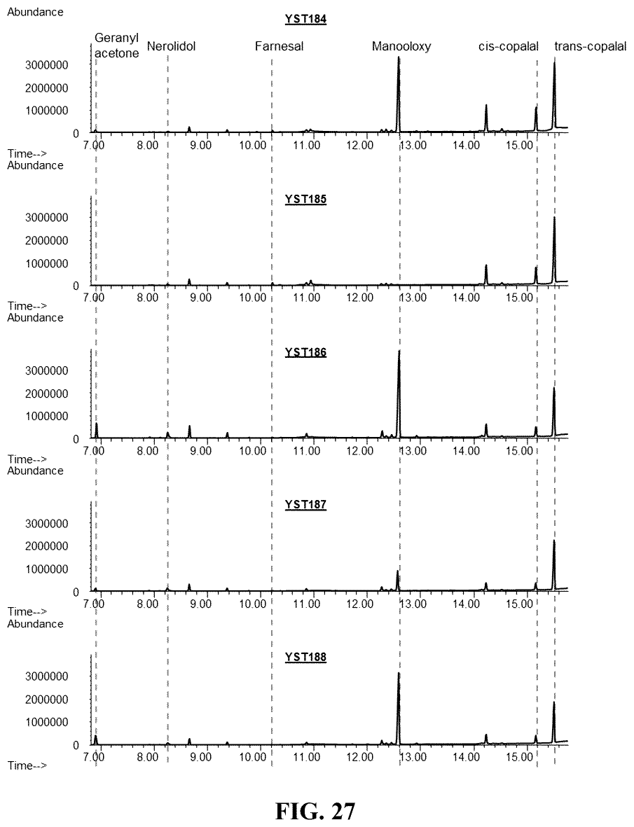

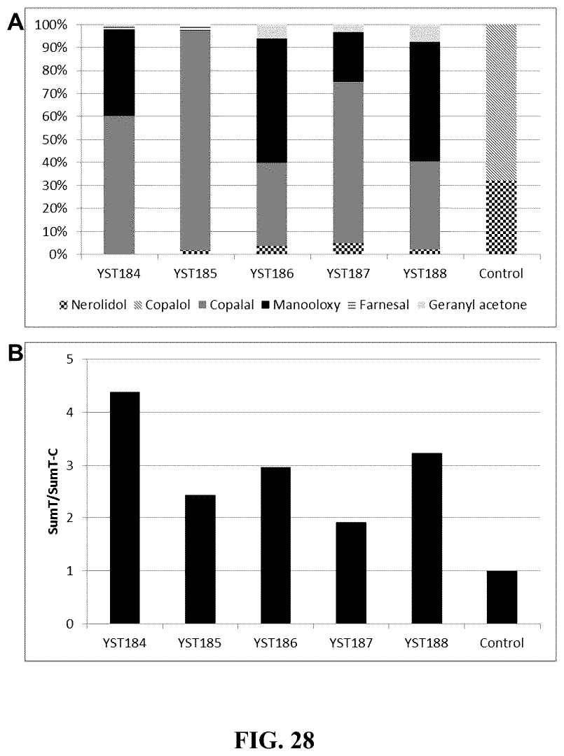

Under the tested conditions, copalal, nerolidol, farnesal, geranyl acetone and manooloxy were identified in all cultures where the enal-cleaving polypeptides were expressed (FIG. 27). As expected, all tested enal-cleaving polypeptides were able to use farnesal or copalal as substrates to produce geranyl acetone and manooloxy, respectively. In the cultures of YST184, YST185, YST186, YST187 and YST188, manooloxy represented 37%, 1%, 54%, 22% and 52%, respectively, of the sum of identified terpenes (FIG. 28A).

Interestingly, the total amount of identified terpenes in cultures from strains containing the alcohol dehydrogenase and the different enal-cleaving polypeptides were two- to four-folds higher than that of the control culture (FIG. 28B

### Example 18: In Vivo Gamma-Ambryl Acetate Production in Saccharomyces cerevisiae Cells Using SCH23-ADH1 from Hyphozyma roseonigra, AspWeDUF4334 from Aspergillus wentii and Different Baeyer-Villiger Monooxygenases (BVMOs)

For the production of gamma-ambryl acetate, the genes encoding for the GGPP synthase carG (from Blakeslea trispora, NCBI accession JQ289995.1), the copalyl-pyrophosphate synthase SmCPS2 (from Salvia miltiorrhiza, NCBI accession ABV57835.1), the copalyl-pyrophosphate phosphatase TalVeTPP (from Talaromyces verruculosus, NCBI accession KUL89334.1), the alcohol dehydrogenase SCH23-ADH1 (from Hyphozyma roseonigra), the enal-cleaving polypeptide AspWeDUF4334 (from Aspergillus wentii; GenBank accession OJJ34591.1) and one of the tested Baeyer-Villiger monooxygenases (BVMOs) were expressed in the engineered Saccharomyces cerevisiae strain YST075 as described in general methods.

Three BVMOs were evaluated:
SCH23-BVMO1 (from Hyphozyma roseonigra) (SEQ ID NO: 2).
SCH24-BVMO1 (from Filobasidum magnum) (SEQ ID NO: 6).
AspWeBVMO (from Aspergillus wentii; GenBank accession OJJ34587.1) (SEQ ID NO: 16).

All genes were codon optimized for their expression in S. cerevisiae (SCH23-BVMO1, SEQ ID NO: 4; SCH24-BVMO1, SEQ ID NO: 8; and AspWeBVMO, SEQ ID NO: 18).

The obtained strains were termed YST190 (with SCH23-BVMO1), YST191 (with SCH24-BVMO1) and YST192 (with AspWeBVMO). These strains were cultivated as described in the general methods section above: the production of manooloxy and other compounds was identified using GC-MS analysis.

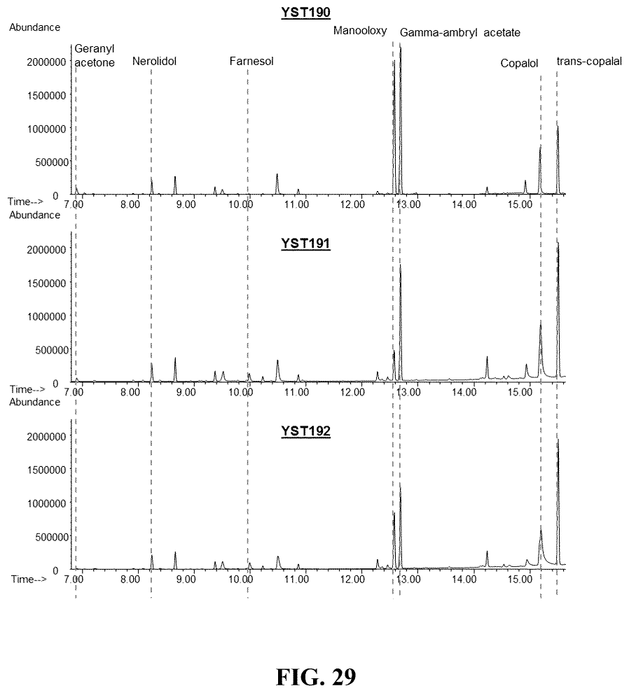

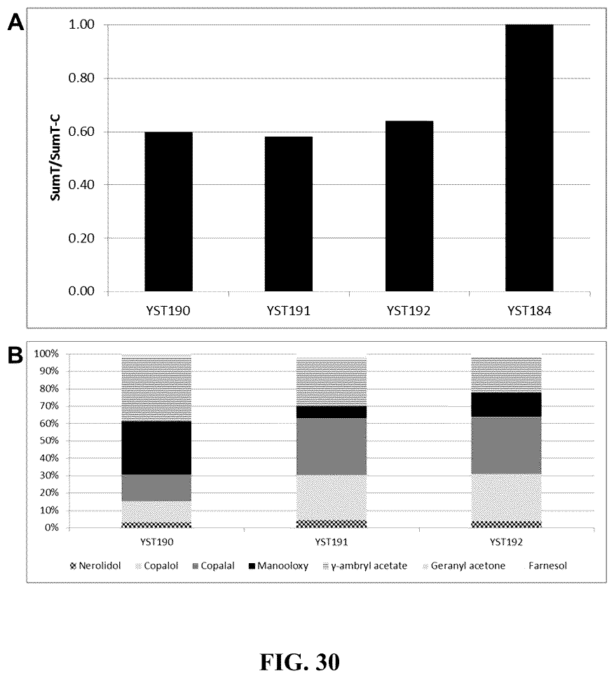

Under the tested conditions, copalol, copalal, nerolidol, farnesol, geranyl acetone, manooloxy and gamma-ambryl acetate were identified in all cultures (FIG. 29). Interestingly, and different from previous experiments, the conversion of copalol to copalal was not complete. In addition, when compared with a strain not harboring BVMOs, the total amount of terpenes produced was lower (FIG. 30A). In the cultures of YST190, YST191 and YST192, gamma-ambryl acetate represented 37%, 27% and 20%, respectively, of the sum of identified terpenes (FIG. 30B).

### Example 19: In-Vivo Production of Sclareol Oxide Using a Labdendiol Biosynthesis Pathway and a Carbon-Carbon Bound Enal-Cleaving Polypeptide

In this experiment, the plasmid pJ401-LOH-2 (described above) was used to transform the DP1205 E. coli cells creating a background strain producing labdendiol ((13E)-13-Labdene-8,15-diol) as described above.

This strain was then co-transformed with a second plasmid carrying a codon optimized nucleotide sequence encoding for an alcohol dehydrogenase and an enzyme with enal-cleaving polypeptideenal-cleaving polypeptide activity:
pJ423-AzetolADH1, containing an optimized DNA sequence encoding for the alcohol dehydrogenase AzetolADH1; and
pJ423-SCH94-3944-3945, containing optimized DNA sequences encoding for the alcohol dehydrogenase SCH94-3944 and the enal-cleaving polypeptide SCH94-3945.

The transformed cells were cultivated and the formation of terpene derivatives was analysed by GC-MS as described above.

When cells were transformed with the vector pJ401-LOH-2 and with an empty pJ423 vector formation of labdendiol was observed (FIG. 31).

When cells were transformed with the vector pJ401-LOH-2 and with pJ423-AzetolADH1 to co-express an alcohol dehydrogenase, formation of two new products were observed (FIG. 31). NMR analysis confirmed the two compounds as being two isomers of (+)-8,13-epoxy-labdan-15-al (compounds 7a and 7b) as shown in the scheme below. These two compounds result from the instability of 8-hydroxy-labd-13-en-15-al (6) produced by the oxidation of labdendiol. A postulated mechanism of dehydration and rearrangement of compound 6 to compound 7a and 7b is shown in the scheme below.

When cells were transformed with the vector pJ401-LOH-2 and with pJ423-SCH94-3944-3945 to co-express an alcohol dehydrogenase and a enal-cleaving polypeptide, formation of sclareol oxide was observed in addition to compounds 7a and 7b. The formation of sclareol oxide in the presence of a enal-cleaving polypeptide can be explained by the transformation steps shown in the scheme below. The SCH94-3944 enal-cleaving polypeptide catalyses the C—C double bond cleavage of compound 6 to the 8-Hydroxy-14,15-bisnorlabdan-13-one (8). Compound 8 is unstable and is converted under mild conditions to sclareol oxide (Barrero et al., Tetrahedron 49, (45) 1993, 10405-10412; Hua et al., Tetrahedron 67 (6) 2011, 1142-1144). The relative small final amounts of sclareol oxide relative to compounds 7a and 7b is due to the competition between the enzymatic activity of the SCH94-3944 and the chemical dehydration of compound 6.

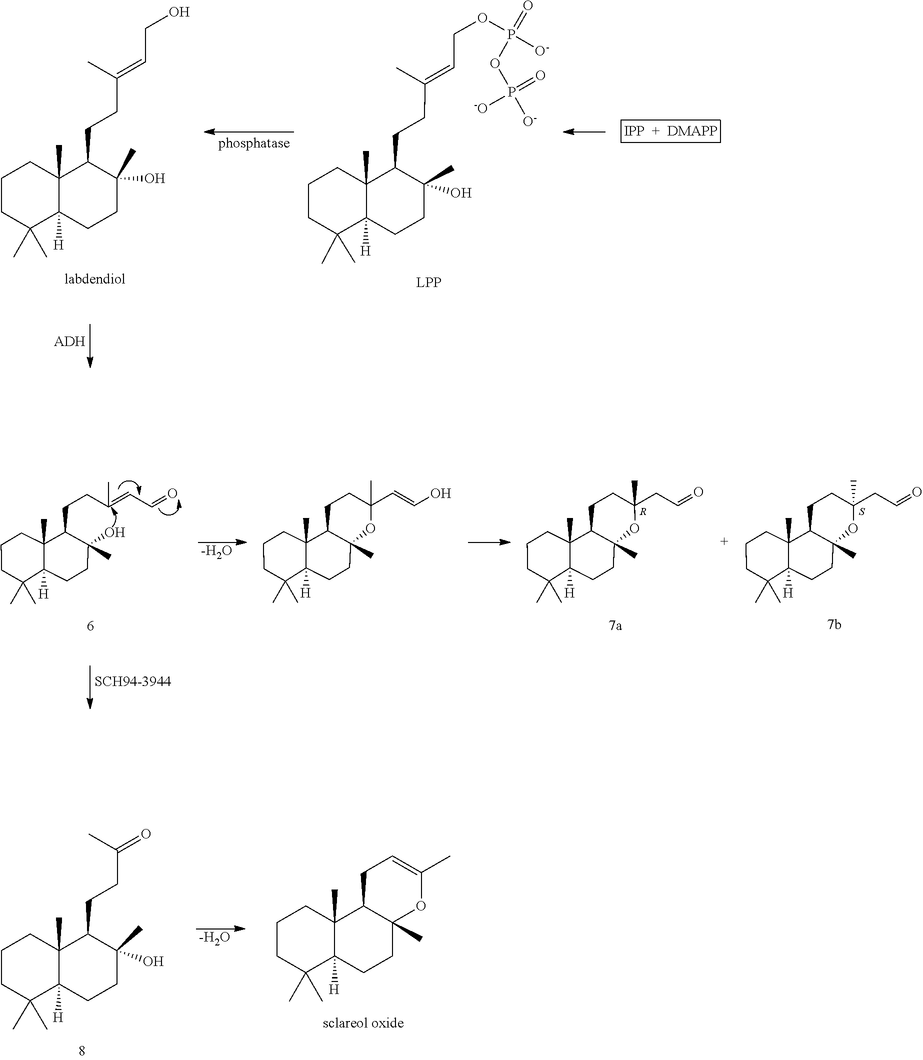

### Example 20. In Vivo Gamma-Ambrol Production in Saccharomyces cerevisiae Cells Using SCH23-ADH1 from Hyphozyma roseonigra, AspWeDUF4334 from Aspergillus wentii, SCH23-BVMO1 from Hyphozyma roseonigra and Different Esterases

For the production of gamma-ambrol, the genes encoding for the bifunctional enzyme PvCPS (from Talaromyces verruculosus), the copalyl-pyrophosphate phosphatase TalVeTPP (from Talaromyces veruculosum), the alcohol dehydrogenase SCH23-ADH1 (from Hyphozyma roseonigra), the enal-cleaving AspWeDUF4334 (from Aspergillus wentii), the BVMO SCH23-BVMO1 (from Hyphozyma roseonigra) and one of the tested esterases (EST) were expressed in the engineered Saccharomyces cerevisiae strain YST075 as described in general methods.

Two esterases were evaluated:
SCH23-EST1 (from Hyphozyma roseonigra).
SCH24-EST1 (from Cryptococcus albidus).

All genes were codon optimized for their expression in S. cerevisiae (SCH23-EST, SEQ ID NO: 22; SCH24-EST, SEQ ID NO: 26).

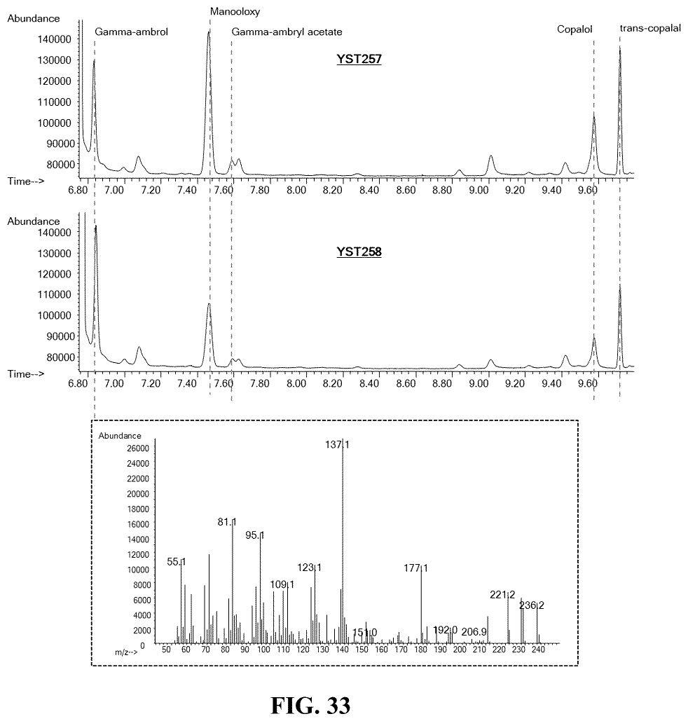

The obtained strains were termed YST257 (with SCH23-EST) and YST258 (with SCH24-EST). These strains were cultivated as described in general methods. Under the tested conditions, nerolidol, copalol, copalal, manooloxy, gamma-ambryl acetate and gamma-ambrol were identified in all cultures using GC-MS/FID analysis (FIG. 33). In the cultures of YST257 and YST258, gamma-ambrol represented 16% and 29%, respectively, of the sum of identified terpenes.

### Example 21: In-Vivo Production of γ-Ambrol by Combining the Enal-Cleaving Activity, the BVMO Activity and the Esterase Activity in E. coli Cells

A first vector was designed containing two operons each under the control of a T5 promoter. The first operon contains two cDNAs encoding for:
The AspWeTPP phosphatase from Aspergillus wentii (SEQ ID NO: 170) (GenBank accession OJJ34585.1); and
PvCPS, a copalyl-diphosphate synthase from Talaromyces verruculosus (SED ID NO: 173) (GenBank accession BBF88128.1). PvCPS catalyzes the production of copalyl PP from IPP and DMAPP.

The cDNAs encoding for AspWeTPP and PvCPS were codon optimized (SEQ ID NOs: 171 and 174) and the operon was designed containing the two cDNAs and an RBS sequence (AAGGAGGTAAAAAA) (SEQ ID NO: 196) placed upstream of each the CDNAs.

The second operon contains two cDNAs encoding for:
SCH94-3944, an enal-cleaving polypeptide from Rhodococcus (SEQ ID NO: 34),
SCH94-3945, an alcohol dehydrogenase from Rhodococcus (SEQ ID NO: 161).

The cDNAs encoding for SCH94-3945 and SCH94-3944 were codon optimized (SEQ ID NOs: 162 and 35) and the operon was designed containing the two cDNAs and an RBS sequence (AAGGAGGTAAAAAA) (SEQ ID NO: 196) placed upstream of each the cDNAs.

The two operons were assembled in a single vector, providing pJ401-Mnoxy allowing to express all gene of the biosynthetic pathway from FPP to manooloxy.

Bacteria cells (DP1205) were co-transformed with the plasmid pJ401-Manoxy and with a second plasmid:
pJ423-SCH24-BVMO carrying a gene encoding for a BVMO, SCH24-BVMO (SEQ ID NO: 7) alone,
pJ423-SCH24-BVMO-SCH24-EST, containing an operon composed of cDNA encoding for a BVMO, SCH24-BVMO1 (SEQ ID NO: 7), and a cDNA encoding for an esterase, SCH24-EST (SEQ ID NO: 25),
or a control plasmid pJ423.

The transformed cells were cultivated and the production terpenes was analysed as described above under the conditions described in the experimental section.

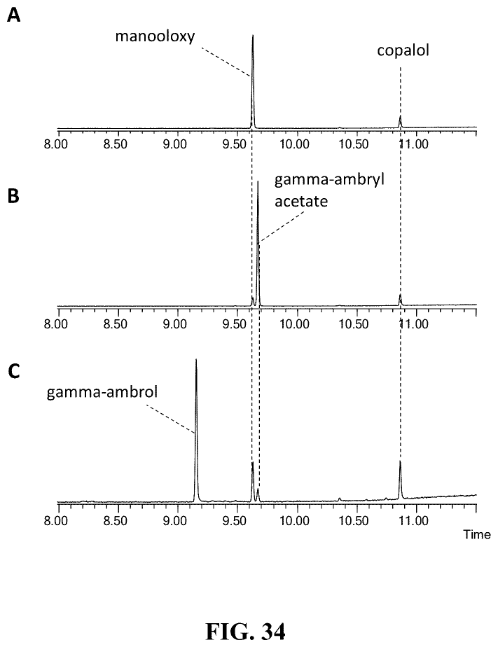

When cells were transformed with the vector pJ401-Mnoxy and with an empty pJ423 vector, formation of only manooloxy was observed. (FIG. 34-A).

When cells were transformed with the vector pJ401-Mnoxy and with pJ423-SCH24-BVMO, formation of γ-ambryl acetate was observed (FIG. 34-B).

When cells were transformed with the vector pJ401-Mnoxy and with pJ423-SCH24-BVMO-SCH24-EST, formation of γ-ambrol was observed (FIG. 34-B).

This experiment shows that the following pathway can be introduced in a host cell to produce gamma-ambrol.

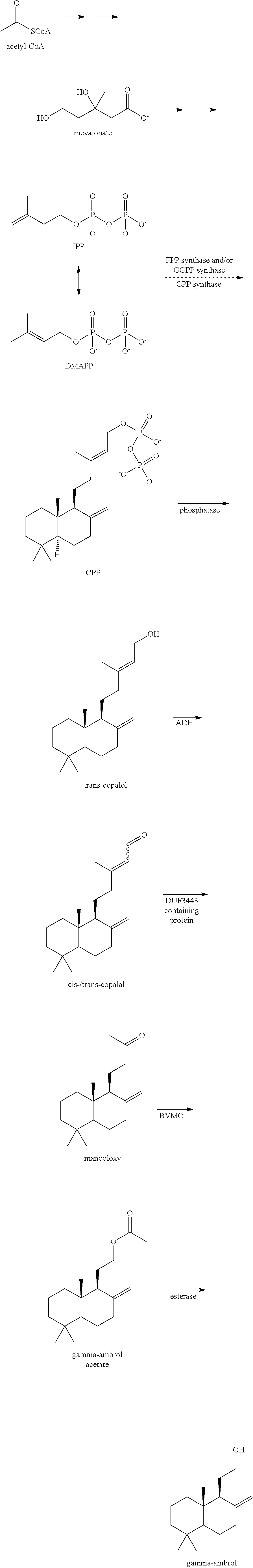

The content of any document cross-referenced herein is incorporated by reference.

TABLE
Overview of Sequences

| | | | |
|---|---|---|---|
| SEQ | | | |
| ID | Description | Source | Type |

BVMOs

| | | | |
|---|---|---|---|
| 1 | SCH23-BVMO1_wt | Hyphozyma roseonigra | NA |
| 2 | SCH23-BVMO1_wt | Hyphozyma roseonigra | AA |
| 3 | SCH23-BVMO1_E. coli | Hyphozyma roseonigra | NA |
| | optimized | | |
| 4 | SCH23-BVMO1_Yeast | Hyphozyma roseonigra | NA |
| | optimized | | |
| 5 | SCH24-BVMO1_wt | Filobasidium magnum | NA |
| 6 | SCH24-BVMO1_wt | Filobasidium magnum | AA |
| 7 | SCH24-BVMO1_E. coli | Filobasidium magnum | NA |
| | optimized | | |
| 8 | SCH24-BVMO1_Yeast | Filobasidium magnum | NA |
| | optimized | | |
| 9 | SCH25-BVMO1_wt | Papiliotrema laurentii | NA |
| 10 | SCH25-BVMO1_wt | Papiliotrema laurentii | AA |
| 11 | SCH25-BVMO1_E. coli | Papiliotrema laurentii | NA |
| | optimized | | |
| 12 | SCH46-BVMO1_wt | Bensingtonia ciliata | NA |
| 13 | SCH46-BVMO1_wt | Bensingtonia ciliata | AA |
| 14 | SCH46-BVMO1_E. coli | Bensingtonia ciliata | NA |
| | optimized | | |
| 15 | AspWeBVMO_wt | Aspergillus wentii | NA |
| 16 | AspWeBVMO_wt | Aspergillus wentii | AA |
| | (OJJ34587.1) | | |
| 17 | AspWeBVMO_E. coli | Aspergillus wentii | NA |
| | optimized | | |
| 18 | AspWeBVMO_Yeast | Aspergillus wentii | NA |
| | optimized | | |

Esterases

| | | | |
|---|---|---|---|
| 19 | SCH23-EST_wt | Hyphozyma roseonigra | NA |
| 20 | SCH23-EST_wt | Hyphozyma roseonigra | AA |
| 21 | SCH23-EST_E. coli | Hyphozyma roseonigra | NA |
| | optimized | | |
| 22 | SCH23-EST_Yeast | Hyphozyma roseonigra | NA |
| | optimized | | |
| 23 | SCH24-EST_wt | Filobasidium magnum | NA |
| 24 | SCH24-EST_wt | Filobasidium magnum | AA |
| 25 | SCH24-EST_E. coli | Filobasidium magnum | NA |
| | optimized | | |
| 26 | SCH24-EST_Yeast | Filobasidium magnum | NA |
| | optimized | | |
| 27 | SCH25-EST_wt | Papiliotrema laurentii | NA |
| 28 | SCH25-EST_wt | Papiliotrema laurentii | AA |
| 29 | SCH25-EST_E. coli | Papiliotrema laurentii | NA |
| | optimized | | |
| 30 | SCH46-EST_wt | Bensingtonia ciliata | NA |
| 31 | SCH46-EST_wt | Bensingtonia ciliata | AA |
| 32 | SCH46-EST_E. coli | Bensingtonia ciliata | NA |
| | optimized | | |

Enal-cleaving polypeptides

| 33 | SCH94-3944_wt | Rhodococcus erythropolis | NA |
|---|---|---|---|
| 34 | SCH94-3944_wt | Rhodococcus erythropolis | AA |
| 35 | SCH94-3944_E. coli | Rhodococcus erythropolis | NA |
| | optimized | | |
| 36 | SCH94-3944_Yeast | Rhodococcus erythropolis | NA |
| | optimized | | |
| 37 | SCH80-05241_wt | Rhodococcus rhodochrous | NA |
| 38 | SCH80-05241_wt | Rhodococcus rhodochrous | AA |
| 39 | SCH80-05241_E. coli | Rhodococcus rhodochrous | NA |
| | optimized | | |
| 40 | SCH80-05241_Yeast | Rhodococcus rhodochrous | NA |
| | optimized | | |
| 41 | Pdigit7033_wt | Penicillium digitatum | NA |
| 42 | Pdigit7033_wt | Penicillium digitatum | AA |
| 43 | Pdigit7033_E. coli | Penicillium digitatum | NA |
| | optimized | | |
| 44 | Pdigit7033_Yeast | Penicillium digitatum | NA |
| | optimized | | |
| 45 | PitalDUF4334-1_wt | Penicillium italicum | NA |
| | (JQGA01001114.1 | | |
| | 71518-72084 (+)) | | |
| 46 | PitalDUF4334-1_wt | Penicillium italicum | AA |
| | (KGO69886.1) | | |
| 47 | PitalDUF4334-1_E. coli | Penicillium italicum | NA |
| | optimized | | |
| 48 | AspWe DUF4334_wt | Aspergillus wentii | NA |
| | (LISE01000065.1 | | |
| | (263404 to 263924)) | | |
| 49 | AspWe DUF4334_wt | Aspergillus wentii | AA |
| | (OJJ43591) | | |
| 50 | AspWe DUF4334_E. coli | Aspergillus wentii | NA |
| | optimized | | |
| 51 | AspWe DUF4334_Yeast | Aspergillus wentii | NA |
| | optimized | | |
| 52 | RhoagDUF4334-2_wt | Rhodococcus hoagii strain | NA |
| | (NZ_LWTW01000167.1 | PAM2288 | |
| | 18658-19134 (−)) | | |
| 53 | RhoagDUF4334-2_wt | Rhodococcus hoagii strain | AA |
| | (WP_005516054) | PAM2288 | |
| 54 | RhoagDUF4334-2_E. coli | Rhodococcus hoagii strain | NA |
| | optimized | PAM2288 | |
| 55 | RhoagDUF4334-3_wt | Rhodococcus hoagii strain | NA |
| | (NZ_LRQY01000021.1 | N128 | |
| | 163210-163686 (−)) | | |
| 56 | RhoagDUF4334-3_wt | Rhodococcus hoagii strain | AA |
| | (WP_013414658) | N128 | |
| 57 | RhoagDUF4334-3_E. coli | Rhodococcus hoagii strain | NA |
| | optimized | N128 | |
| 58 | RhoagDUF4334-4_wt | Rhodococcus hoagii | NA |
| | (NZ_BCRL01000037.1 | | |
| | 133790-134266 (+)) | | |
| 59 | RhoagDUF4334-4_wt | Rhodococcus hoagii | AA |
| | (WP_022593671) | | |
| 60 | RhoagDUF4334-4_E. coli | | NA |
| | optimized | | |
| 61 | CnecaDUF4334_wt | Cupriavidus necator | NA |
| | (CP002879.1: | | |
| | 512553-513138) | | |
| 62 | CnecaDUF4334_wt | Cupriavidus necator | AA |
| | (WP_049800708.1) | | |
| 63 | CnecaDUF4334_E. coli | Cupriavidus necator | NA |
| | optimized | | |
| 64 | CnecaDUF4334_Yeast | Cupriavidus necator | NA |
| | optimized | | |
| 65 | PitalDUF4334-2_wt | Penicillium italicum | NA |
| | (JQGA01000120.1 | | |
| | 65652-66635 (+)) | | |
| 66 | PitalDUF4334-2_wt | Penicillium italicum | AA |
| | (KGO77618.1) | | |
| 67 | PitalDUF4334-2_E. coli | Penicillium italicum | NA |
| | optimized | | |
| 68 | Rins-DUF4334_wt | Ralstonia insidiosa | NA |
| | (NZ_PKPC01000002.1 | | |
| | 18273-18773 (−)) | | |
| 69 | Rins-DUF4334_wt | Ralstonia insidiosa | AA |
| | (WP_104654734) | | |
| 70 | Rins-DUF4334_E. coli | Ralstonia insidiosa | NA |
| | optimized | | |
| 71 | CgatDUF4334_wt | Cryptococcus gattii | NA |
| | | EJ B2 | |
| 72 | CgatDUF4334_wt | Cryptococcus gattii | AA |
| | (KIR80015) | EJ B2 | |
| 73 | CgatDUF4334_E. coli | Cryptococcus gattii | NA |
| | optimized | EJ B2 | |
| 74 | GclavDUF4334_wt | Grosmannia clavigera | NA |
| | (XM_014316402.1) | kw1407 | |
| 75 | GclavDUF4334_wt | Grosmannia clavigera | AA |
| | (XP_014171877.1) | kw1407 | |
| 76 | GclavDUF4334_E. coli | Grosmannia clavigera | NA |
| | optimized | kw1407 | |
| 77 | OmaiusDUF4334_wt | Oidiodendron maius Zn | NA |
| | (KN832882.1 | | |
| | 673187-675938 (−)) | | |
| 78 | OmaiusDUF4334_wt | Oidiodendron maius Zn | AA |
| | (KIM97275) | | |
| 79 | OmaiusDUF4334_E. coli | Oidiodendron maius Zn | NA |
| | optimized | | |
| 80 | TcurvaDUF4334_wt | Thermomonospora | NA |
| | (NC_013510.1) | curvata | |
| 81 | Tcurva DUF4334_wt | Thermomonospora | AA |
| | (WP_012851400.1) | curvata | |
| 82 | TcurvaDUF4334_E. coli | Thermomonospora | NA |
| | optimized | curvata | |
| 83 | DlitoDUF4334_wt | Pseudomonas litoralis | NA |
| | (NZ_LT629748.1 | | |
| | 3096922-3097413 (+)) | | |
| 84 | DlitoDUF4334_wt | Pseudomonas litoralis | AA |
| | (WP_090274689) | | |
| 85 | DlitoDUF4334_E. coli | Pseudomonas litoralis | NA |
| | optimized | | |
| 86 | PprotDUF4334_wt | Pseudomonas protegens | NA |
| | (NC_021237.1 | | |
| | 5528027-5528524 (−)) | | |
| 87 | PprotDUF4334_wt | Pseudomonas protegens | AA |
| | (WP_015636872.1) | | |
| 88 | PprotDUF4334_E. coli | Pseudomonas protegens | NA |
| | optimized | | |
| 89 | SCH94-3944-W44A_variant | artificial | AA |
| 90 | SCH94-3944-W44A_E. coli | artificial | NA |
| | optimized | | |
| 91 | SCH94-3944-T51A_variant | artificial | AA |
| 92 | SCH94-3944-T51A_E. coli | artificial | NA |
| | optimized | | |
| 93 | SCH94-3944-H53A_variant | artificial | AA |
| 94 | SCH94-3944-H53A_E. coli | artificial | NA |
| | optimized | | |
| 95 | SCH94-3944-L59A_variant | artificial | AA |
| 96 | SCH94-3944-L59A_E. coli | artificial | NA |
| | optimized | | |
| 97 | SCH94-3944-W64A_variant | artificial | AA |
| 98 | SCH94-3944-W64A_E. coli | artificial | NA |
| | optimized | | |
| 99 | SCH94-3944-K67A_variant | artificial | AA |
| 100 | SCH94-3944-K67A_E. coli | artificial | NA |
| | optimized | | |
| 101 | SCH94-3944-S71A_variant | artificial | AA |
| 102 | SCH94-3944-S71A_E. coli | artificial | NA |
| | optimized | | |
| 103 | SCH94-3944-R106A_variant | artificial | AA |
| 104 | SCH94-3944-R106A_E. coli | artificial | NA |
| | optimized | | |
| 105 | SCH94-3944-Y115A_variant | artificial | AA |
| 106 | SCH94-3944-Y115A_E. coli | artificial | NA |
| | optimized | | |
| 107 | SCH94-3944-D116A_variant | artificial | AA |
| 108 | SCH94-3944-D116A_E. coli | artificial | NA |
| | optimized | | |
| 109 | SCH94-3944-D122A_variant | artificial | AA |
| 110 | SCH94-3944-D122A_E. coli | artificial | NA |
| | optimized | | |
| 111 | SCH94-3944-M136A_variant | artificial | AA |
| 112 | SCH94-3944-M136A_E. coli | artificial | NA |
| | optimized | | |
| 113 | SCH94-3944-K139A_variant | artificial | AA |
| 114 | SCH94-3944-K139A | artificial | NA |
| | E. coli optimized | | |
| 115 | SCH94-3944-F152A_variant | artificial | AA |
| 116 | SCH94-3944-F152A_E. coli | artificial | NA |
| | optimized | | |
| 117 | SCH94-3944-L154A_variant | artificial | AA |
| 118 | SCH94-3944-L154A_E. coli | artificial | NA |
| | optimized | | |
| 119 | SCH94-3944-R156A_variant | artificial | AA |
| 120 | SCH94-3944-R156A_E. coli | artificial | NA |
| | optimized | | |

Cassettes and primers

| | | | |
|---|---|---|---|
| 121 | Integration cassette fragment 1 | artificial | NA |
| 122 | Integration cassette fragment 2 | artificial | NA |
| 123 | Integration cassette fragment 3 | artificial | NA |
| 124 | LEU2 yeast marker_primer 1 | artificial | NA |
| 125 | LEU2 yeast marker_primer 2 | artificial | NA |
| 126 | AmpR E. coli marker_primer 1 | artificial | NA |
| 127 | AmpR E. coli marker_primer 2 | artificial | NA |
| 128 | Yeast origin of | artificial | NA |
| | replication_primer 1 | | |
| 129 | Yeast origin of | artificial | NA |
| | replication_primer 2 | | |
| 130 | E. coli replication | artificial | NA |
| | origin_primer 1 | | |
| 131 | E. coli replication | artificial | NA |
| | origin_primer 2 | | |
| 132 | DNA fragment for S. cerevisiae | artificial | NA |
| | co-transformation | | |

ADHs

| | | | |
|---|---|---|---|
| 133 | SCH23-ADH1_wt | Hyphozyma roseonigra | NA |
| 134 | SCH23-ADH1_wt | Hyphozyma roseonigra | AA |
| 135 | SCH23-ADH1_Yeast optimized | Hyphozyma roseonigra | NA |
| 136 | SCH23-ADH2_wt | Hyphozyma roseonigra | NA |
| 137 | SCH23-ADH2_wt | Hyphozyma roseonigra | AA |
| 138 | SCH23-ADH2_Yeast optimized | Hyphozyma roseonigra | NA |
| 139 | SCH24-ADH1_wt | Filobasidium magnum | NA |
| 140 | SCH24-ADH1_wt | Filobasidium magnum | AA |
| 141 | SCH24-ADH1_Yeast optimized | Filobasidium magnum | NA |
| 142 | SCH24-ADH2_wt | Filobasidium magnum | NA |
| 143 | SCH24-ADH2_wt | Filobasidium magnum | AA |
| 144 | SCH24-ADH2_Yeast optimized | Filobasidium magnum | NA |
| 145 | RrhSecADH_wt | Rhodococcus sp. | NA |
| 146 | RrhSecADH_wt | Rhodococcus sp. | AA |
| | (WP_043801412.1) | | |
| 147 | RrhSecADH_E.coli optimized | Rhodococcus sp. | NA |
| 148 | SCH80-00043_wt | Rhodococcus rhodochrous | NA |
| 149 | SCH80-00043_wt | Rhodococcus rhodochrous | AA |
| 150 | SCH80-00043_E. coli optimized | Rhodococcus rhodochrous | NA |
| 151 | SCH80-04254_wt | Rhodococcus rhodochrous | NA |
| 152 | SCH80-04254_wt | Rhodococcus rhodochrous | AA |
| 153 | SCH80-04254_E. coli optimized | Rhodococcus rhodochrous | NA |
| 154 | SCH80-06135_wt | Rhodococcus rhodochrous | NA |
| 155 | SCH80-06135_wt | Rhodococcus rhodochrous | AA |
| 156 | SCH80-06135_E. coli optimized | Rhodococcus rhodochrous | NA |
| 157 | SCH80-06582_wt | Rhodococcus rhodochrous | NA |
| 158 | SCH80-06582_wt | Rhodococcus rhodochrous | AA |
| 159 | SCH80-06582_E. coli optimized | Rhodococcus rhodochrous | NA |
| 160 | SCH94-03945_wt | Rhodococcus erythropolis | NA |
| 161 | SCH94-03945_wt | Rhodococcus erythropolis | AA |
| 162 | SCH94-03945_E. coli optimized | Rhodococcus erythropolis | NA |
| 163 | SCH80-05240_wt | Rhodococcus rhodochrous | NA |
| 164 | SCH80-05240_wt | Rhodococcus rhodochrous | AA |
| 165 | SCH80-05240_E. coli | Rhodococcus rhodochrous | NA |
| | optimized | | |
| 166 | AzeTolADH1_wt | Azoarcus toluclasticus | NA |
| | (NZ_KB899498.1 | | |
| | 215502-216629 (+)) | | |
| 167 | AzTolADH1_wt | Azoarcus toluclasticus | AA |
| | (WP_018990713.1) | | |
| 168 | AzTolADH1_E. coli optimized | Azoarcus toluclasticus | NA |

Other sequences

| | | | |
|---|---|---|---|
| 169 | AspWeTPP_wt (OJJ34585.1) | Aspergillus wentii | NA |
| 170 | AspWeTPP_wt | Aspergillus wentii | AA |
| | (KV878213.1:2482776- | | |
| | 2483627) | | |
| 171 | AspWeTPP_E. coli optimized | Aspergillus wentii | NA |
| 172 | PvCPS_wt (LC316181.1) | Talaromyces verruculosus | NA |
| 173 | PvCPS_wt (BBF88128.1) | Talaromyces verruculosus | AA |
| 174 | PvCPS_E. coli optimized | Talaromyces verruculosus | NA |
| 175 | TalCeTPP_wt | Talaromyces cellulolyticus | NA |
| | (BBPS01001258.1 | | |
| | (16027-16959)) | | |
| 176 | TalCeTPP_wt (GAM42000.1) | Talaromyces cellulolyticus | AA |
| 177 | TalCeTPP_E. coli optimized | Talaromyces cellulolyticus | NA |
| 178 | CdGeoA_wt | Castellaniella defragrans | NA |
| 179 | CdGeoA_wt | Castellaniella defragrans | AA |
| | (WP_043683915.1) | | |
| 180 | CdGeoA_E. coli optimized | Castellaniella defragrans | NA |
| 181 | GGPP synthase carG_wt | Blakeslea trispora | NA |
| | (AFC92798.1) | | |
| 182 | GGPP synthase carG_wt | Blakeslea trispora | AA |
| | (JQ289995.1) | | |
| 183 | GGPP synthase carG_Yeast | Blakeslea trispora | NA |
| | optimized | | |
| 184 | SmCPS2_wt (EU003997.1 | Salvia miltiorrhiza | NA |
| | 73-2454 (+)) | | |
| 185 | SmCPS2_Yeast optimized | Salvia miltiorrhiza | AA |
| 186 | SmCPS2_Yeast optimized | Salvia miltiorrhiza | NA |
| 187 | SsLPS_wt (JN133923.1) | Salvia sciarea | NA |
| 188 | SsLPS_wt (AET21247.1) | Salvia sciarea | AA |
| 189 | SsLPS_E. coli optimized | Salvia sciarea | NA |
| 190 | CrtE_wt | Pantoea agglomerans | NA |
| | (M38424.1 40-963 (+)) | | |
| 191 | CrtE_wt (AAA24819.1) | Pantoea agglomerans | AA |
| 192 | CrtE_Yeast optimized | Pantoea agglomerans | NA |
| 193 | TalVeTPP_wt | Talaromyces verruculosus | NA |
| | (LHCL01000010.1 | | |
| | 150095-151030 (+)) | | |
| 194 | TalVeTPP_wt (KUL89334.1) | Talaromyces verruculosus | AA |
| 195 | TalVeTPP_Yeast optimized | Talaromyces verruculosus | NA |
| 196 | RBS sequence | artificial | AA |
| 197 | BVMO sequence motif l | artificial | AA |
| 198 | BVMO sequence motif 2 | artificial | AA |
| 199 | BVMO sequence motif 3 | artificial | AA |
| 200 | BVMO sequence motif 4 | artificial | AA |
| 201 | BVMO sequence motif 5 | artificial | AA |
| 202 | BVMO sequence motif 6 | artificial | AA |
| 203 | BVMO sequence motif 7 | artificial | AA |
| 204 | BVMO sequence motif 8 | artificial | AA |
| 205 | Enal-cleaving polypeptide | artificial | AA |
| | sequence motif 1 | | |
| 206 | Enal-cleaving polypeptide | artificial | AA |
| | sequence motif 2 | | |
| 207 | Enal-cleaving polypeptide | artificial | AA |
| | sequence motif 3 | | |
| 208 | Enal-cleaving polypeptide | artificial | AA |
| | sequence motif 4 | | |

SEQ ID NO 1: Hyphozyma roseonigra SCH23-BVMO1 wt
ATGCCTTCCGCAATCACCCCGCCGGTTGATCATCGCAGTCTTCCAGGTCTTTTCAAGCCACAGAGG
AAGCTCAAAGTGATATGTGTCGGAGCCGGCGCCTCGGGCTTACTTCTTTCCTACAAGATACAACGA
CACTTCGAGGATTTCGAGCTCCAAGTCTTTGAGAAGAATCCCGAGGTATCAGGAACCTGGTACGA
GAACAGGTATCCCGGCTGCGCTTGTGATGTTCCCTCGCATAATTATACATGGTCTTTTGAGCCCAA
AACCGACTGGTCCGCCAACTATGCATCATCGAAGGAGATTTTCAAATATTTCAAGGACTTCACGAG
GAAATATGGTCTAAGCAAGTACATCAAGCTGGAACATGAGGTCGTGGGAGCCACGTGGATGGAGG
CGGAGGCACAGTGGAAAGTTGACGTCAAGGACCTTCGAAGTGGAAACACGCAGAGCTCGTTTGCG
CATATACTGGTCAATGCAGGAGGCATTTTGAATGCTTGGCGATACCCGCCAATTCCAGGAATCAAG
GATTTCAAGGGTGATCTTGTTCACTCCGCAGCTTGGCCAGAACATCTTGATCTTAATGGGAAGGTT
GTCGGTCTCATCGGAAATGGATCCTCCGGCATTCAGATCCTTCCGGCCATCAAGAAGGATGTAAAG
CAACTCGTTACATTCATTCGGGAAGCAACTTGGGTGGCGCCTCCTTTAGGTCAAGCCTATCGTGCG
TTCTCGACTGATGAACAGGCTCAGTTTGCGCAAGATCCGCGCCATCACCTGGAGACACGGCGTGCA
ACTGAGGCTACCATGAATCAATCATTTGGTATCTTCCATTCGGGATCCGAGGAGCAGAAAGGGGTT
CGCCAATATATGCAGAATATCATGGAAACGAAGCTCAACAACAAACAGCTCGAGAGTGTGCTGAT
TCCTGAGTGGTCGGTCGGTTGTCGGCGTCTTACACCAGGTACTAATTATCTAGAATCCCTGTCAGA
CGACAATGTCAAGGTCGTCTATGGTGAGATCACACAAATTACCGAATCGGGTGTCATCTGCGATGA
TGGTAAAGGCGAATATCCCGTTGAAGTTCTTATTTGCGCCACCGGCTTCGACACCACCTTCAAACC
ACGATTCCCACTCATCGGTACAACCCAAGAGAAACTCAGTGATGTTTGGAAAGATGATCCGAGGG
GCTACTTTGGGATCGCAACCAACAACTATCCCAACTACTTCTTCACTCTTGGACCAAATTGTCCAAT
CGGTAATGGCCCCGTGCTGTGTGCCATTGAAGCTGAGGTTGAATATATAATCAACATGCTCTCGAA
GTTTCAGAAGGAGAACATTCGTTCTTTTGATATCAAGGCAGATGCTGTCGACGCCTTCAACGACTG
GAAGGATGACTTCATGAAAGATACCATCTGGGCAGAACAATGCCGGTCATGGTACAAGGCAGGAT
CCGCCACCGGTAAAATCCTTGCATTGTGGCCAGGCTCGACTTTGCACTACTTGGAAGCACTCAAGT
CGCCGCGGTGGGAGGACTGGGACTTCAAGTATCAGCCTGGTAGAAATCGTTTCCACTACTTTGGAA
ATGGTCATAGCTGTGCTGAGCAGGATGGCGATCTGAGCTGGTACATTCGCAACGAGGATGATTCTT
ATATTGATCCGGTACTCAAGCCGAAGCCGAAGGCAGCAGTTGAAAGCGAGGCACATATCGCCCTG
CCAGGAATCGGTCCGATGTTGATGGAAGACCCGCGTGATGTTGCTGTAGAGGCCTAG
SEQ ID NO 2: Hyphozyma roseonigra SCH23-BVMO1 wt
MPSAITPPVDHRSLPGLFKPQRKLKVICVGAGASGLLLSYKIQRHFEDFELQVFEKNPEVSGTWYENRY
PGCACDVPSHNYTWSFEPKTDWSANYASSKEIFKYFKDFTRKYGLSKYIKLEHEWGATWMEAEAQW
KVDVKDLRSGNTQSSFAHILVNAGGILNAWRYPPIPGIKDFKGDLVHSAAWPEHLDLNGKVVGLIGNG
SSGIQILPAIKKDVKQLVTFIREATWVAPPLGQAYRAFSTDEQAQFAQDPRHHLETRRATEATMNQSFG
IFHSGSEEQKGVRQYMQNIMETKLNNKQLESVLIPEWSVGCRRLTPGTNYLESLSDDNVKVVYGEITQI
TESGVICDDGKGEYPVEVLICATGFDTTFKPRFPLIGTTQEKLSDVWKDDPRGYFGIATNNYPNYFFTLG
PNCPIGNGPVLCAIEAEVEYIINMLSKFQKENIRSFDIKADAVDAFNDWKDDFMKDTIWAEQCRSWYK
AGSATGKILALWPGSTLHYLEALKSPRWEDWDFKYQPGRNRFHYFGNGHSCAEQDGDLSWYIRNEDD
SYIDPVLKPKPKAAVESEAHIALPGIGPMLMEDPRDVAVEA
SEQ ID NO 3: Hyphozyma roseonigra SCH23-BVMO1 E. coli optimized
ATGCCGTCTGCCATTACTCCACCTGTTGATCACCGTTCCCTGCCGGGCCTGTTTAAACCGCAGCGCA
AGCTGAAAGTGATTTGCGTGGGCGCGGGTGCGAGCGGCCTGCTGTTGAGCTACAAGATTCAGCGC
CACTTCGAAGATTTCGAGCTGCAAGTGTTTGAGAAGAACCCTGAAGTTAGCGGTACGTGGTACGA
GAACCGTTATCCGGGTTGTGCGTGCGATGTGCCGAGCCATAACTACACCTGGAGCTTTGAGCCGAA
AACGGATTGGTCCGCCAATTATGCGAGCAGCAAAGAGATTTTCAAATATTTCAAAGATTTTACGCG
TAAATATGGTCTGTCTAAATACATTAAATTGGAACATGAAGTGGTCGGCGCGACCTGGATGGAAG
CCGAGGCGCAGTGGAAAGTTGACGTTAAAGATCTGCGCAGCGGTAACACCCAGTCCAGCTTCGCG
CATATCCTGGTTAACGCCGGCGGCATTCTGAATGCCTGGCGTTATCCGCCGATTCCGGGCATCAAA
GATTTCAAGGGTGACCTGGTGCATAGCGCAGCATGGCCGGAGCATTTGGACCTGAATGGCAAAGT
CGTTGGTCTGATCGGCAACGGTAGCAGCGGTATCCAAATCCTGCCGGCAATTAAGAAAGACGTGA
AGCAACTGGTGACGTTTATCCGTGAAGCCACCTGGGTCGCACCGCCGCTGGGTCAAGCGTACCGTG
CGTTTTCCACCGACGAGCAAGCACAGTTTGCGCAGGACCCGCGCCACCACCTGGAAACCCGTCGT
GCGACCGAAGCCACCATGAATCAGAGCTTTGGTATTTTCCATAGCGGCAGCGAAGAACAGAAAGG
TGTCCGCCAGTACATGCAAAACATTATGGAAACCAAGCTGAATAATAAGCAACTGGAGAGCGTCC
TGATTCCGGAGTGGAGCGTCGGCTGTCGTCGTCTGACCCCGGGCACGAACTACCTGGAAAGCCTG
AGCGACGACAATGTCAAAGTTGTGTACGGTGAGATTACCCAAATCACCGAGAGCGGTGTCATCTG
CGATGACGGCAAGGGTGAGTATCCGGTTGAAGTCCTGATCTGCGCCACCGGTTTTGATACGACCTT
TAAACCGCGCTTCCCGCTGATCGGTACGACCCAGGAAAAGCTGAGCGACGTGTGGAAAGATGATC
CGCGCGGTTACTTCGGCATCGCGACGAATAATTATCCGAACTATTTCTTCACGCTGGGTCCGAACT
GCCCGATCGGTAATGGCCCGGTCCTGTGTGCGATCGAAGCCGAAGTTGAGTACATCATCAACATGC
TGAGCAAGTTTCAGAAAGAAAATATTCGCTCCTTCGACATTAAAGCCGACGCGGTGGACGCGTTTA
ATGATTGGAAAGACGATTTCATGAAAGATACCATCTGGGCAGAACAGTGCCGTAGCTGGTACAAG
GCCGGCAGCGCGACCGGCAAGATTCTGGCACTGTGGCCGGGCAGCACGCTGCACTACCTGGAAGC
GCTGAAAAGCCCGCGTTGGGAAGATTGGGACTTCAAGTATCAACCGGGCCGTAACCGTTTCCACT
ACTTTGGCAACGGTCACAGCTGTGCCGAGCAAGATGGCGACCTGTCCTGGTACATCCGTAATGAA
GATGACAGCTACATTGACCCGGTTCTGAAACCGAAGCCGAAAGCCGCGGTGGAGAGCGAGGCACA
CATCGCACTGCCGGGTATTGGCCCGATGCTGATGGAAGATCCGCGTGATGTCGCGGTTGAGGCGTA
A
SEQ ID NO 4: Hyphozyma roseonigra SCH23-BVMO1 Yeast optimized
ATGCCATCTGCTATCACTCCACCAGTTGACCACAGATCTTTGCCAGGTTTGTTCAAGCCACAAAGA
AAGTTGAAGGTTATCTGTGTTGGTGCTGGTGCTTCTGGTTTGTTGTTGTCTTACAAGATCCAAAGAC
ACTTCGAAGACTTCGAATTGCAAGTTTTCGAAAAGAACCCAGAAGTTTCTGGTACTTGGTACGAAA
ACAGATACCCAGGTTGTGCTTGTGACGTTCCATCTCACAACTACACTTGGTCTTTCGAACCAAAGA
CTGACTGGTCTGCTAACTACGCTTCTTCTAAGGAAATCTTCAAGTACTTCAAGGACTTCACTAGAA
AGTACGGTTTGTCTAAGTACATCAAGTTGGAACACGAAGTTGTTGGTGCTACTTGGATGGAAGCTG
AAGCTCAATGGAAGGTTGACGTTAAGGACTTGAGATCTGGTAACACTCAATCTTCTTTCGCTCACA
TCTTGGTTAACGCTGGTGGTATCTTGAACGCTTGGAGATACCCACCAATCCCAGGTATCAAGGACT
TCAAGGGTGACTTGGTTCACTCTGCTGCTTGGCCAGAACACTTGGACTTGAACGGTAAGGTTGTTG
GTTTGATCGGTAACGGTTCTTCTGGTATCCAAATCTTGCCAGCTATCAAGAAGGACGTTAAGCAAT
TGGTTACTTTCATCAGAGAAGCTACTTGGGTTGCTCCACCATTGGGTCAAGCTTACAGAGCTTTCTC
TACTGACGAACAAGCTCAATTCGCTCAAGACCCAAGACACCACTTGGAAACTAGAAGAGCTACTG
AAGCTACTATGAACCAATCTTTCGGTATCTTCCACTCTGGTTCTGAAGAACAAAAGGGTGTTAGAC
AATACATGCAAAACATCATGGAAACTAAGTTGAACAACAAGCAATTGGAATCTGTTTTGATCCCA
GAATGGTCTGTTGGTTGTAGAAGATTGACTCCAGGTACTAACTACTTGGAATCTTTGTCTGACGAC
AACGTTAAGGTTGTTTACGGTGAAATCACTCAAATCACTGAATCTGGTGTTATCTGTGACGACGGT
AAGGGTGAATACCCAGTTGAAGTTTTGATCTGTGCTACTGGTTTCGACACTACTTTCAAGCCAAGA
TTCCCATTGATCGGTACTACTCAAGAAAAGTTGTCTGACGTTTGGAAGGACGACCCAAGAGGTTAC
TTCGGTATCGCTACTAACAACTACCCAAACTACTTCTTCACTTTGGGTCCAAACTGTCCAATCGGTA
ACGGTCCAGTTTTGTGTGCTATCGAAGCTGAAGTTGAATACATCATCAACATGTTGTCTAAGTTCC
AAAAGGAAAACATCAGATCTTTCGACATCAAGGCTGACGCTGTTGACGCTTTCAACGACTGGAAG
GACGACTTCATGAAGGACACTATCTGGGCTGAACAATGTAGATCTTGGTACAAGGCTGGTTCTGCT
ACTGGTAAGATCTTGGCTTTGTGGCCAGGTTCTACTTTGCACTACTTGGAAGCTTTGAAGTCTCCAA
GATGGGAAGACTGGGACTTCAAGTACCAACCAGGTAGAAACAGATTCCACTACTTCGGTAACGGT
CACTCTTGTGCTGAACAAGACGGTGACTTGTCTTGGTACATCAGAAACGAAGACGACTCTTACATC
GACCCAGTTTTGAAGCCAAAGCCAAAGGCTGCTGTTGAATCTGAAGCTCACATCGCTTTGCCAGGT
ATCGGTCCAATGTTGATGGAAGACCCAAGAGACGTTGCTGTTGAAGCTTAA
SEQ ID NO 5: Filobasidium magnum SCH24-BVMO1 wt
ATGACTATCGATTTGCAGCAGCCCGACGCCGTGCCATTCACCTCTTCGACTTTTGTCGTGCCGGATC
CATCGAACCTGGCCTCTCAGGCACAGAATTCACAGCTCCAATCTGCTCAAGAAGGAGCAGAGTAC
CCTGTGAACGCACATGGGGTTCGAGGAGATGGAACGATTCATGAACGACCGATCAACGATCGCAG
GAAGATGCGCGTCATCTGCGTCGGTGCAGGCATCTCAGGTCTCTATATGGCCATCAAGCTCCCTCG
AAGTACGGAAAATGTAGAGCTCAAGATCTACGAGAAGAATCACGATCTCGGTGGGACCTGGCTGG
AGAATAGGTATCCAGGATGCGCTTGTGATGTACCAGCCCATGCCTACGCATACAGCTTCGAGAAC
AACCCCGAATTTCCTAGATTCTTTTCGAGCTCGGAAGACATCCACAAGTACTTGCTTCGCGTGGCT
GATAAATATGATTGCAAGAAATACATCGCATTCAACACCAAAGTAGTCGAGGCCATTTGGGACGA
AGAACAGGGCATCTATAACGTCAAGATTGAACGCTCGGATGGCACAGTATTCCAGGACACGTGCG
AAGTTCTATTGAACGCTTCTGGTATCCTTAACGCCTGGAGGTACCCTGGGATTCCTGGAATTAAGG
ACTACAAGGGCACGTTAATGCACTCGGCTACCTGGGACCGATCCGTGTCCCTGAAAGGCAAAAAG
GTTGCCCTCATCGGATCAGGATCATCAGGCATTCAGATCTTGCCCAACATCCTTGACGATTGCAAA
GAGGTCGTGACATACATCATTGATCCAGCCTGGATTGCCCCTGCGAATCTTGTCACGGCTGGAGTC
TCGGACGACGGTGAAGAGCCTAAGGAGCCGACGCCTGAAGAATTGGCGTCGAGTAGTGACTTCGC
CTACTCGCAAGAGCAAATCAATGGCTTCAAGAAGGACCCTAAGTCACTGATGGATCATCGAGCAA
CGCTCGAAAGGACGATGAATCAGTCTTTCCCCATCTTACTCAGAGGCTCACCGTCCAACCTTTATG
CCGCTTCTCTCTTTGAAGACCTGATGAGGAAACGCCTTGCCAAGAAGCCTGAGGTAGCGGATGCCA
TCATCCCCGAATGGTCAATCGGTTGCCGACGTCTCACTCCTGGACCACACTATCTTGAGGCCTTGT
GCAATCCCAAGGTCAAGATCTTGACCCAAGCTATCAAGTCCTTCTCCGATAAGGGAATGTACACTG
CCGATGGCGAACACGAAGACTTTGACGTGGTGATATGCGCGACTGGATTCGACGTATCGTTCCGAC
CCCGATTCAAATTTATCGGCAAGGACGGGTATGAGGTGCCCGAGAACTTTGGTCAGACTCCCAAA
GGTTACCTCGCTCTCGCTTACGCCGGTTTCCCTAATTCGTTCATCTTCATGGGGCCGAACGGACCTA
TCGCCAACGGATCTGTCGTGGTCTCCCTGGAGAAACAAGGCGACTACTTCATCAAGGCGATCAAC
AAGATCCAAAGGCAGAATATAAAAGGCATGACTGTCAGATTCGATGCGGTCGATGATTTCACCAA
CCACGTAGACAAATACATGGATAGGACCGTGCTCACCGATGACTGCATCAGCTGGTACAAGAACG
GGAAACGAGACGGACGAGTCAGTGCCGTCTGGCCTGGGAGCGCACTTCATTATATGGAGGCCATC
GCCGACCCTAGATGGGAGGATTACACCTACACTTATCGCGAACCCGGTCATTCTTTTTCGTTCTTGG
GAGATGGGACGTCCTGGGTCGAACACACCGGAGGAGACACGGCTTGGTACCTGAAAGAGACCCTC
TAA
SEQ ID NO 6: Filobasidium magnum SCH24-BVMO1 wt
MTIDLQQPDAVPFTSSTFVVPDPSNLASQAQNSQLQSAQEGAEYPVNAHGVRGDGTIHERPINDRRKM
RVICVGAGISGLYMAIKLPRSTENVELKIYEKNHDLGGTWLENRYPGCACDVPAHAYAYSFENNPEFP
RFFSSSEDIHKYLLRVADKYDCKKYIAFNTKVVEAIWDEEQGIYNVKIERSDGTVFQDTCEVLLNASGIL
NAWRYPGIPGIKDYKGTLMHSATWDRSVSLKGKKVALIGSGSSGIQILPNILDDCKEVVTYIIDPAWIAP
ANLVTAGVSDDGEEPKEPTPEELASSSDFAYSQEQINGFKKDPKSLMDHRATLERTMNQSFPILLRGSPS
NLYAASLFEDLMRKRLAKKPEVADAIIPEWSIGCRRLTPGPHYLEALCNPKVKILTQAIKSFSDKGMYT
ADGEHEDFDVVICATGFDVSFRPRFKFIGKDGYEVPENFGQTPKGYLALAYAGFPNSFIFMGPNGPIAN
GSVVVSLEKQGDYFIKAINKIQRQNIKGMTVRFDAVDDFTNHVDKYMDRTVLTDDCISWYKNGKRDG
RVSAVWPGSALHYMEAIADPRWEDYTYTYREPGHSFSFLGDGTSWVEHTGGDTAWYLKETL
SEQ ID NO 7: Filobasidium magnum SCH24-BVMO1 E. coli optimized
ATGACCATCGATTTGCAACAGCCAGACGCAGTCCCGTTTACGAGCAGCACTTTCGTCGTACCGGAC
CCGTCCAACCTGGCATCCCAGGCTCAAAACAGCCAACTGCAGAGCGCGCAAGAGGGCGCAGAGTA
CCCGGTGAATGCACACGGTGTCCGCGGTGACGGCACCATTCACGAGCGTCCGATCAATGACCGTC
GTAAAATGCGCGTCATCTGCGTTGGTGCGGGTATTAGCGGCCTGTATATGGCGATCAAACTGCCGC
GCAGCACCGAGAATGTTGAACTGAAGATCTACGAGAAAAACCATGACCTCGGCGGCACGTGGCTG
GAGAATCGCTACCCTGGCTGCGCGTGCGATGTTCCGGCGCATGCGTATGCATATTCTTTTGAGAAT
AATCCGGAATTTCCACGCTTTTTCAGCAGCAGCGAGGATATCCACAAGTACCTGTTGCGTGTTGCG
GACAAGTACGACTGTAAGAAATACATCGCCTTTAACACCAAAGTCGTTGAGGCTATCTGGGACGA
AGAACAGGGTATTTACAATGTGAAGATTGAGCGTAGCGACGGCACCGTGTTCCAGGACACCTGTG
AGGTGCTGCTGAACGCGAGCGGTATTCTGAATGCCTGGCGCTACCCGGGCATCCCTGGCATTAAGG
ATTACAAAGGTACGCTGATGCACAGCGCTACCTGGGACCGTAGCGTTTCTTTGAAAGGCAAAAAA
GTCGCACTGATTGGCAGCGGTAGCAGCGGTATCCAGATTCTGCCGAACATTCTGGACGACTGCAA
AGAAGTGGTCACGTACATTATCGACCCGGCGTGGATTGCTCCGGCTAACCTGGTGACCGCGGGTGT
CTCCGATGATGGTGAGGAACCGAAAGAGCCAACCCCTGAGGAACTGGCCTCATCCTCCGACTTCG
CTTATAGCCAGGAACAGATTAACGGCTTCAAGAAAGATCCGAAGTCGCTGATGGATCACCGCGCC
ACGCTGGAGCGTACCATGAATCAATCGTTTCCGATTCTGCTGCGTGGCTCTCCGAGCAACTTGTAT
GCCGCAAGCCTGTTCGAGGATTTGATGCGTAAGCGTCTGGCGAAGAAGCCGGAAGTTGCGGACGC
GATTATCCCGGAGTGGAGCATCGGTTGCAGACGCCTGACGCCGGGTCCGCATTACCTGGAAGCAC
TGTGTAACCCGAAAGTGAAGATCCTGACTCAGGCGATCAAGAGCTTTAGCGATAAGGGCATGTAT
ACTGCGGACGGTGAGCATGAAGATTTCGATGTTGTCATTTGTGCGACCGGTTTCGATGTGAGCTTT
CGTCCGCGCTTCAAGTTTATTGGTAAAGATGGCTATGAAGTCCCAGAGAATTTCGGCCAAACGCCG
AAAGGTTATCTGGCACTGGCGTACGCCGGCTTCCCGAACAGCTTCATCTTTATGGGTCCGAACGGT
CCGATTGCGAACGGTAGCGTTGTGGTGAGCCTGGAGAAGCAAGGTGACTACTTCATTAAAGCGAT
CAATAAGATCCAGCGTCAAAACATTAAGGGTATGACCGTTCGTTTCGACGCCGTGGATGATTTTAC
GAATCACAGTGGACAAATACATGGACCGTACGGTGCTGACCGACGATTGCATCAGCTGGTACAAG
AATGGTAAACGTGACGGTCGTGTTAGCGCAGTTTGGCCGGGTTCCGCGCTGCACTATATGGAAGCC
ATCGCAGACCCGCGTTGGGAAGATTACACCTACACCTATCGCGAACCGGGTCACTCTTTTAGCTTC
CTGGGTGATGGCACCAGCTGGGTTGAGCATACGGGTGGCGATACCGCCTGGTATTTGAAAGAAAC
CCTGTAA
SEQ ID NO 8: Filobasidium magnum SCH24-BVMO1 Yeast optimized
ATGACTATCGACTTGCAACAACCAGACGCTGTTCCATTCACTTCTTCTACTTTCGTTGTTCCAGACC
CATCTAACTTGGCTTCTCAAGCTCAAAACTCTCAATTGCAATCTGCTCAAGAAGGTGCTGAATACC
CAGTTAACGCTCACGGTGTTAGAGGTGACGGTACTATCCACGAAAGACCAATCAACGACAGAAGA
AAGATGAGAGTTATCTGTGTTGGTGCTGGTATCTCTGGTTTGTACATGGCTATCAAGTTGCCAAGA
TCTACTGAAAACGTTGAATTGAAGATCTACGAAAAGAACCACGACTTGGGTGGTACTTGGTTGGA
AAACAGATACCCAGGTTGTGCTTGTGACGTTCCAGCTCACGCTTACGCTTACTCTTTCGAAAACAA
CCCAGAATTCCCAAGATTCTTCTCTTCTTCTGAAGACATCCACAAGTACTTGTTGAGAGTTGCTGAC
AAGTACGACTGTAAGAAGTACATCGCTTTCAACACTAAGGTTGTTGAAGCTATCTGGGACGAAGA
ACAAGGTATCTACAACGTTAAGATCGAAAGATCTGACGGTACTGTTTTCCAAGACACTTGTGAAGT
TTTGTTGAACGCTTCTGGTATCTTGAACGCTTGGAGATACCCAGGTATCCCAGGTATCAAGGACTA
CAAGGGTACTTTGATGCACTCTGCTACTTGGGACAGATCTGTTTCTTTGAAGGGTAAGAAGGTTGC
TTTGATCGGTTCTGGTTCTTCTGGTATCCAAATCTTGCCAAACATCTTGGACGACTGTAAGGAAGTT
GTTACTTACATCATCGACCCAGCTTGGATCGCTCCAGCTAACTTGGTTACTGCTGGTGTTTCTGACG
ACGGTGAAGAACCAAAGGAACCAACTCCAGAAGAATTGGCTTCTTCTTCTGACTTCGCTTACTCTC
AAGAACAAATCAACGGTTTCAAGAAGGACCCAAAGTCTTTGATGGACCACAGAGCTACTTTGGAA
AGAACTATGAACCAATCTTTCCCAATCTTGTTGAGAGGTTCTCCATCTAACTTGTACGCTGCTTCTT
TGTTCGAAGACTTGATGAGAAAGAGATTGGCTAAGAAGCCAGAAGTTGCTGACGCTATCATCCCA
GAATGGTCTATCGGTTGTAGAAGATTGACTCCAGGTCCACACTACTTGGAAGCTTTGTGTAACCCA
AAGGTTAAGATCTTGACTCAAGCTATCAAGTCTTTCTCTGACAAGGGTATGTACACTGCTGACGGT
GAACACGAAGACTTCGACGTTGTTATCTGTGCTACTGGTTTCGACGTTTCTTTCAGACCAAGATTCA
AGTTCATCGGTAAGGACGGTTACGAAGTTCCAGAAAACTTCGGTCAAACTCCAAAGGGTTACTTG
GCTTTGGCTTACGCTGGTTTCCCAAACTCTTTCATCTTCATGGGTCCAAACGGTCCAATCGCTAACG
GTTCTGTTGTTGTTTCTTTGGAAAAGCAAGGTGACTACTTCATCAAGGCTATCAACAAGATCCAAA
GACAAAACATCAAGGGTATGACTGTTAGATTCGACGCTGTTGACGACTTCACTAACCACGTTGACA
AGTACATGGACAGAACTGTTTTGACTGACGACTGTATCTCTTGGTACAAGAACGGTAAGAGAGAC
GGTAGAGTTTCTGCTGTTTGGCCAGGTTCTGCTTTGCACTACATGGAAGCTATCGCTGACCCAAGA
TGGGAAGACTACACTTACACTTACAGAGAACCAGGTCACTCTTTCTCTTTCTTGGGTGACGGTACT
TCTTGGGTTGAACACACTGGTGGTGACACTGCTTGGTACTTGAAGGAAACTTTGTAA
SEQ ID NO 9: Papiliotrema laurentii SCH25-BVMO1 wt
ATGCCTTCCGCAATCACCCCGCCGGTTGATCATCGCAGTCTTCCAGGTCTTTTCAAGCCACAGAGG
AAGCTCAAAGTGATATGTGTCGGAGCCGGCGCCTCGGGCTTACTTCTTTCCTACAAGATACAACGA
CACTTCGAGGATTTCGAGCTCCAAGTCTTTGAGAAGAATCCTGAAGTATCAGGAACCTGGTACGAG
AACAGATATCCCGGCTGCGCTTGTGATGTTCCCTCGCATAATTATACATGGTCTTTTGAGCCCAAA
ACCGACTGGTCCGCCAACTATGCATCATCGAAGGAGATTTTCAAATATTTCAAGGACTTCACGAAG
AAGTATGGTCTTAGCAAGTACATCAAGCTGGAGCATGAGGTCGTGGGGGCCACGTGGATGGAGGC
GGAGGCACAGTGGAAAGTTGACGTCAAGGACCTTCGAAGTGGAAACACGCAGAGCTCGTTTGCGC
ATATACTGGTCAATGCAGGAGGCATTCTGAATGCTTGGCGATATCCGCCAATTCCAGGAATCAAGG
ATTTCAAGGGTGATCTTGTCCACTCCGCAGCTTGGCCAGAACATCTTGATCTTAATGGGAAGGTTG
TCGGTCTCATCGGAAATGGATCCTCCGGCATTCAGATCCTTCCGGCCATCAAGAAGGATGTAAAGC
AACTCGTTACATTCATTCGGGAAGCAACTTGGGTGGCGCCTCCTTTAGGTCAAGCCTATCGTGCGT
TCTCGACTGATGAACAGGCTCAGTTTGCGCAAGATCCGCGCCATCACCTGGAGACACGGCGTGCA
ATTGAGGCTACCATGAATCAATCATTTGGTATCTTCCATTCGGGATCCGAGGAGCAGAAAGGGGTT
CGCCAATATATGCAGAATATCATGGAAACGAAGCTCAACAACAAACAGCTCGAGAGTGTGCTGAT
TCCTGAGTGGTCGGTCGGTTGTCGGCGTCTTACACCAGGTACTAATTACCTAGAATCCCTGTCGGA
CGACAATGTCAAGGTCGTCTACGGTGAGATCACACAAATTACCGAATTGGGTGTCATCTGCGATGA
TGGCAAAGGCGAGTATCCCGTTGAAGTTCTTATTTGCGCCACTGGCTTCGACACCACCTTCAAACC
ACGATTCCCACTCATCGGTACAACCCAAGAGAAACTCAGTGATGTTTGGAAAGATGATCCGAGGG
GTTACTTCGGGATTGCAACCAACAACTATCCCAACTACTTCTTCACTCTTGGACCGAATTGTCCAAT
CGGTAATGGCCCCGTGCTGTGTGCCATCGAAGCTGAGGTTGATTATATAATCAACATGCTCTCAAA
GTTTCAAATGGAGAACATTCGTTCTTTTGATATCAAGGCAGATGCTGTCGACGCCTTCAACGACTG
GAAGGATGACTTCATGAAAGATACCATCTGGGCAGAACAATGCCGGTCATGGTACAAGGCAGGAT
CTGCCACCGGTAAAATCCTTGCATTGTGGCCAGGCTCGACTTTGCACTACTTGGAAGCACTCAAGT
CGCCGCGGTGGGAGGATTGGGACTTCAAGTATCAGCCTGGTAGAAATCGTTTCCACTACTTTGGAA
ATGGTCATAGCTGTGCTGAGCAGGATGGCGATCTGAGCTGGTACATTCGCAACGAGGATGATTCTT
ATATTGATCCGGTACTCAAGCCAAAGTCGAAGGCAGCAATTGAGAGCGAGGCACATATCGCCCTG
CCAGGAATCGGTCCGATGTTGATGGAAGACCCGCGTGATGTTGCTGTAGAGGCCTAG
SEQ ID NO 10: Papiliotrema laurentii SCH25-BVMO1 wt
MPSAITPPVDHRSLPGLFKPQRKLKVICVGAGASGLLLSYKIQRHFEDFELQVFEKNPEVSGTWYENRY
PGCACDVPSHNYTWSFEPKTDWSANYASSKEIFKYFKDFTKKYGLSKYIKLEHEVVGATWMEAEAQW
KVDVKDLRSGNTQSSFAHILVNAGGILNAWRYPPIPGIKDFKGDLVHSAAWPEHLDLNGKVVGLIGNG
SSGIQILPAIKKDVKQLVTFIREATWVAPPLGQAYRAFSTDEQAQFAQDPRHHLETRRAIEATMNQSFGI
FHSGSEEQKGVRQYMQNIMETKLNNKQLESVLIPEWSVGCRRLTPGTNYLESLSDDNVKVVYGEITQIT
ELGVICDDGKGEYPVEVLICATGFDTTFKPRFPLIGTTQEKLSDVWKDDPRGYFGIATNNYPNYFFTLGP
NCPIGNGPVLCAIEAEVDYIINMLSKFQMENIRSFDIKADAVDAFNDWKDDFMKDTIWAEQCRSWYKA
GSATGKILALWPGSTLHYLEALKSPRWEDWDFKYQPGRNRFHYFGNGHSCAEQDGDLSWYIRNEDDS
YIDPVLKPKSKAAIESEAHIALPGIGPMLMEDPRDVAVEA
SEQ ID NO 11: Papiliotrema laurentii SCH25-BVMO1 E. coli optimized
ATGCCATCTGCCATTACTCCACCTGTTGATCATCGTAGCCTGCCGGGTCTGTTCAAGCCGCAACGT
AAGTTGAAAGTGATCTGTGTTGGCGCGGGCGCGAGCGGCCTGTTGCTGAGCTACAAGATTCAGCG
TCACTTTGAGGACTTTGAGTTGCAAGTTTTTGAGAAAAACCCTGAAGTGAGCGGCACCTGGTACGA
GAATCGCTACCCGGGTTGCGCGTGCGATGTTCCGAGCCATAACTATACCTGGTCTTTTGAGCCGAA
AACGGATTGGTCCGCAAACTATGCCAGCAGCAAAGAAATTTTCAAGTACTTCAAAGATTTCACCA
AGAAATATGGTCTGTCTAAATACATTAAACTGGAACACGAAGTCGTGGGTGCGACGTGGATGGAA
GCGGAAGCTCAATGGAAAGTTGACGTCAAAGACTTGCGTAGCGGCAACACCCAGAGCTCCTTCGC
GCACATTCTGGTCAATGCCGGTGGCATTCTGAACGCTTGGCGTTACCCGCCGATTCCGGGTATCAA
AGATTTTAAGGGTGACCTGGTGCACTCGGCAGCGTGGCCGGAGCATCTGGATCTGAATGGTAAAG
TCGTTGGCCTGATTGGTAACGGTAGCAGCGGCATCCAAATTCTGCCGGCCATCAAAAAAGACGTG
AAACAACTGGTCACGTTTATCCGTGAGGCCACGTGGGTCGCCCCGCCGCTGGGCCAAGCGTACCG
CGCATTTAGCACCGACGAACAGGCGCAGTTTGCACAAGACCCGCGTCACCATCTGGAAACTCGTC
GCGCGATTGAAGCTACCATGAATCAGAGCTTCGGTATCTTCCACAGCGGTTCAGAGGAACAGAAA
GGTGTGCGTCAGTACATGCAGAATATCATGGAAACGAAATTGAATAACAAACAGCTGGAGAGCGT
GCTGATTCCGGAGTGGTCCGTGGGTTGTCGCCGTCTGACCCCGGGCACGAACTATCTGGAGAGCTT
GAGCGACGATAACGTGAAAGTTGTTTATGGCGAGATCACCCAGATCACCGAGCTGGGTGTGATTT
GCGATGATGGCAAGGGTGAGTACCCGGTCGAAGTGCTGATTTGCGCTACCGGTTTCGACACCACGT
TCAAACCGCGCTTCCCGTTGATTGGCACCACCCAGGAAAAGCTGAGCGACGTCTGGAAAGATGAC
CCTCGCGGTTATTTCGGTATCGCGACCAATAACTACCCGAACTACTTTTTCACCCTGGGTCCGAACT
GCCCGATCGGCAATGGTCCGGTCCTGTGTGCAATCGAAGCTGAAGTGGACTATATCATCAATATGC
TGAGCAAATTTCAGATGGAAAACATTCGCAGCTTCGACATTAAAGCCGACGCAGTTGATGCGTTTA
ACGACTGGAAAGATGACTTTATGAAAGACACCATCTGGGCAGAGCAGTGTCGTTCTTGGTACAAG
GCTGGTTCTGCGACGGGTAAGATTTTGGCACTGTGGCCGGGCAGCACGCTGCATTATCTGGAAGCC
CTGAAAAGCCCACGCTGGGAAGATTGGGACTTCAAGTATCAACCGGGTCGTAATCGCTTTCACTAC
TTCGGTAACGGCCACAGCTGCGCGGAGCAAGATGGTGATCTGTCCTGGTATATCCGTAATGAAGAT
GACAGCTACATTGACCCGGTACTGAAGCCGAAGTCCAAGGCAGCGATCGAGAGCGAAGCACACAT
CGCGCTGCCAGGCATTGGTCCGATGCTGATGGAGGACCCGCGTGACGTTGCGGTTGAGGCATAA
SEQ ID NO 12: Bensinstonia ciliata SCH46-BVMO1 wt
ATGCCTTCCGCAATCACCCCACCGGTTGATCATCGCAGTCTTCCAGGTCTTTTCAAGCCACAGAGG
AAGCTCAAAGTGATATGTGTCGGAGCCGGCGCCTCGGGCTTACTTCTTTCCTACAAGATACAACGA
CACTTCGAGGATTTCGAGCTCCAAGTCTTTGAGAAGAATCCTGAGGTATCAGGAACCTGGTACGAG
AACAGGTATCCCGGCTGTGCTTGTGATGTTCCCTCGCATAATTATACATGGTCTTTTGAGCCCAAA
ACCGACTGGTCCGCCAACTATGCATCATCGAAGGAGATTTTCAAATATTTCAAGGACTTCACGAAG
AAGTATGGTCTTAGCAAGTACATCAAGCTGGAGCATGAGGTCGTGGGGGCCACGTGGATGGAGGC
GGAGGCACAGTGGAAAGTTGACGTCAAGGACCTTCGAAGTGGAAACACGCAGAGCTCGTTTGCGC
ATATACTGGTCAATGCAGGAGGCATTCTGAATGCTTGGCGATATCCGCCAATTCCAGGAATCAAGG
ATTTCAAGGGTGATCTTGTCCACTCCGCAGCTTGGCCAGAACATCTTGATCTTAATGGGAAGGTTG
TCGGTCTAATCGGAAATGGATCCTCCGGCATTCAGATCCTTCCGGCCATCAAGAAGGATGTAAAGC
AACTCGTTACATTTATTCGGGAAGCAACTTGGGTGGCGCCTCCTTTAGGTCAAGCCTATCGTGCGT
TCTCGACTGATGAACAGGCTCAGTTTGCGCAAGATCCGCGCCATCACCTGGAAACACGTCGTGCAA
CTGAGGCTACCATGAATCAATCATTTGGTATCTTCCATTCGGGATCCGAGGAGCAGAAAGGAGTTC
GCCAATATATGCAGGATATCATGGAAACGAAGCTCAACAACAAACAGCTCGAGAGTGTGCTGATT
CCTGAGTGGTCGGTCGGTTGTCGGCGTCTTACACCAGGTACTAATTACCTAGAATCCCTATCGGAC
GACAATGTCAAGGTCGTCTACGGTGAAATCACACAAATTACCGAATCAGGTGTCATCTGCGATGAT
GGTAAAGGCGAATATCCCGTCGAAGTTCTTATTTGCGCCACCGGCTTCGACACCACCTTCAAACCA
CGATTTCCACTCATCGGCACTACGAAAGAGAAGCTCAGTGATGTTTGGAAAGATGATCCGAGGGG
CTACTTTGGGATCGCAACCAACAACTATCCCAACTACTTCTTCACTCTTGGACCGAATTGTCCAATC
GGTAATGGCCCCGTGCTGTGTGCCATTGAAGCTGAGGTTGAATATATAATCAACATGCTCTCGAAG
TTTCAGAAGGAGAACATTCGTTCTTTTGATATCAAGGCAGATGCTGTCGACGCCTTCAACGACTGG
AAGGATGACTTCATGAAAGATACCATCTGGGCAGAACAATGCCGGTCATGGTACAAGGCAGGATC
CGCCACTGGTAAAATCCTTGCATTGTGGCCAGGCTCGACTTTGCACTACTTGGAAGCACTCAAGTC
GCCGCGGTGGGAGGACTGGGACTTCAAGTATCAGCCTGGTAGAAATCGTTTCCATTACTTTGGAAA
TGGTCATAGCTGTGCTGAGCAGGATGGCGATCTGAGCTGGTACATCCGCAACGAGGATGATTCTTA
TATTGATCCGGTACTCAAGCCAAAGCCGAAGGCAGCAGTTGAGAGCGAGGCACATATCGCCCTGC
CAGGAATCGGTCCGATGTTGATGGAAGACCCGCGTGATGTTGCTGTAGAGGCCTAG
SEQ ID NO 13: Bensinstonia ciliata (ATCC 20919) SCH46-BVMO1 wt
MPSAITPPVDHRSLPGLFKPQRKLKVICVGAGASGLLLSYKIQRHFEDFELQVFEKNPEVSGTWYENRY
PGCACDVPSHNYTWSFEPKTDWSANYASSKEIFKYFKDFTKKYGLSKYIKLEHEVVGATWMEAEAQW
KVDVKDLRSGNTQSSFAHILVNAGGILNAWRYPPIPGIKDFKGDLVHSAAWPEHLDLNGKVVGLIGNG
SSGIQILPAIKKDVKQLVTFIREATWVAPPLGQAYRAFSTDEQAQFAQDPRHHLETRRATEATMNQSFG
IFHSGSEEQKGVRQYMQDIMETKLNNKQLESVLIPEWSVGCRRLTPGTNYLESLSDDNVKVVYGEITQI
TESGVICDDGKGEYPVEVLICATGFDTTFKPRFPLIGTTKEKLSDVWKDDPRGYFGIATNNYPNYFFTLG
PNCPIGNGPVLCAIEAEVEYIINMLSKFQKENIRSFDIKADAVDAFNDWKDDFMKDTIWAEQCRSWYK
AGSATGKILALWPGSTLHYLEALKSPRWEDWDFKYQPGRNRFHYFGNGHSCAEQDGDLSWYIRNEDD
SYIDPVLKPKPKAAVESEAHIALPGIGPMLMEDPRDVAVEA
SEQ ID NO 14: Bensinstonia ciliata SCH46-BVMO1 E. coli optimized
ATGCCATCTGCCATTACTCCACCTGTTGATCATCGTAGCCTGCCGGGTCTGTTCAAGCCGCAACGT
AAGTTGAAAGTGATCTGTGTTGGCGCGGGCGCGAGCGGCCTGTTGCTGAGCTACAAGATTCAGCG
TCACTTTGAGGACTTTGAGTTGCAAGTTTTTGAGAAAAACCCTGAAGTGAGCGGCACCTGGTACGA
GAATCGCTACCCGGGTTGCGCGTGCGATGTTCCGAGCCATAACTATACCTGGTCTTTTGAGCCGAA
AACGGATTGGTCCGCAAACTATGCCAGCAGCAAAGAAATTTTCAAGTACTTCAAAGATTTCACCA
AGAAATATGGTCTGTCTAAATACATTAAACTGGAACACGAAGTCGTGGGTGCGACGTGGATGGAA
GCGGAAGCTCAATGGAAAGTTGACGTCAAAGACTTGCGTAGCGGCAACACCCAGAGCTCCTTCGC
GCACATTCTGGTCAATGCCGGTGGCATTCTGAACGCTTGGCGTTACCCGCCGATTCCGGGTATCAA
AGATTTTAAGGGTGACCTGGTGCACTCGGCAGCGTGGCCGGAGCATCTGGATCTGAATGGTAAAG
TCGTTGGCCTGATTGGTAACGGTAGCAGCGGCATCCAAATTCTGCCGGCCATCAAAAAAGACGTG
AAACAACTGGTCACGTTTATCCGTGAGGCCACGTGGGTCGCCCCGCCGCTGGGCCAAGCGTACCG
CGCATTTAGCACCGACGAACAGGCGCAGTTTGCACAAGACCCGCGTCACCATCTGGAAACTCGTC
GCGCGACCGAAGCTACCATGAATCAGAGCTTCGGTATCTTCCACAGCGGTTCAGAGGAACAGAAA
GGTGTGCGTCAGTACATGCAGGATATCATGGAAACGAAATTGAATAACAAACAGCTGGAGAGCGT
GCTGATTCCGGAGTGGTCCGTGGGTTGTCGCCGTCTGACCCCGGGCACGAACTATCTGGAGAGCTT
GAGCGACGATAACGTGAAAGTTGTTTATGGCGAGATCACCCAGATCACCGAGTCCGGTGTGATTT
GCGATGATGGCAAGGGTGAGTACCCGGTCGAAGTGCTGATTTGCGCTACCGGTTTCGACACCACGT
TCAAACCGCGCTTCCCGTTGATTGGCACCACCAAAGAAAAGCTGAGCGACGTCTGGAAAGATGAC
CCTCGCGGTTATTTCGGTATCGCGACCAATAACTACCCGAACTACTTTTTCACCCTGGGTCCGAACT
GCCCGATCGGCAATGGTCCGGTCCTGTGTGCAATCGAAGCTGAAGTGGAGTATATCATCAATATGC
TGAGCAAATTTCAGAAAGAAAACATTCGCAGCTTCGACATTAAAGCCGACGCAGTTGATGCGTTT
AACGACTGGAAAGATGACTTTATGAAAGACACCATCTGGGCAGAGCAGTGTCGTTCTTGGTACAA
GGCTGGTTCTGCGACGGGTAAGATTTTGGCACTGTGGCCGGGCAGCACGCTGCATTATCTGGAAGC
CCTGAAAAGCCCACGCTGGGAAGATTGGGACTTCAAGTATCAACCGGGTCGTAATCGCTTTCACTA
CTTCGGTAACGGCCACAGCTGCGCGGAGCAAGATGGTGATCTGTCCTGGTATATCCGTAATGAAG
ATGACAGCTACATTGACCCGGTACTGAAGCCGAAGCCGAAGGCAGCGGTGGAGAGCGAAGCACA
CATCGCGCTGCCAGGCATTGGTCCGATGCTGATGGAGGACCCGCGTGACGTTGCGGTTGAGGCAT
AA
SEQ ID NO 15: Aspergillus wentii AspWeBVMO wt
ATGACCAAAGACAATACCACATCATTCCCCTCGCACGCCATCTACGAGCCACGCCGGACATTAAA
AGTGCTGGTCATAGGGGCTGGTGCGTCCGGTCTATTATTAGCATACAAACTACAGCGGCACTTTGA
TTGTGTGGAAATCACGGTGTTTGAGAAGAACCCCGCAGTGTCCGGCACTTGGTTTGAGAATCGATA
TCCGGGATGTGCCTGTGACGTTCCTTCGCATTGCTATACATGGTCCTTCGAGCCCAACCCCAACTG
GTCCGCCAACTACGCTGGAGCCGACGAGATTCGACAATACTTTGTCGATTTCTGCCATCGCCACGA
CTTGCAGAAATATATCCATCTGGAACATGAGGTGGTCCACGCAGCGTGGAAGTCGGAGACTGGCC
ACTGGGAGGTGCAAGTGCGCGATATACAACACAATTCTCACACACAGCATACTGCGCATATCTTG
ATTAATGCTACTGGAATACTGAATCAATGGAAGTGGCCATCCATTCCCGGATTACAGTCGTTCCAG
GGAGATCTTTTGCACAGTGCAGCATGGGACTCGTCAGTCAATCTAGAGGATAAAACGGTCGCTGTC
ATTGGAAACGGATCATCCGGAATCCAGATTGTCCCAGCGATTCTACCCCAAGTGCGCAAACTCGTG
CACTTTACTCGTCAAGCGGCATGGGTCGCACCTCCAGTCAATGAAGAGTATCAGGAATACTCGCCC
GAACAGATCGAACGCTTTCGCTCAGACCCAACATACCTGCTTGGGGTTCGTCGACAGATTGAAGCA
CGGATGAACGGCTCATTTCTGAAATTCATCCAAGGCTCAGACATGCAACGTCGTGCACACGAGTAT
GTCATGCTGCACATGATGAAGAGACTGGACGGAGACGCCTCCCTGGCAGAGACCTTGGTACCAAC
CTTCCCATTTGGCTGTCGAAGACCGACGCCAGGAACCGGGTATCTCGAAGCACTGAAGGACTCGA
AAGTGGAAACAATTACCGGAGCCCGAATCGCGAATGTGACGGGTAACCAGGTGGTCCTCGAGAAT
GGCACGTCGTATACGGTGGATGCGATTGTGTGCGCCACGGGATTCGATACGTCTTACAAACCACGA
TTCCCACTGGTCGGCAGAGACAGCACCACTCTCAGCGAGGCCTGGAAGGACGAAGTGTCTGCATA
TCTGGGGCTTACAGTTCCTGGATTTCCCAACTATTTTTCCATCTTGGGACCGAACTGTCCGGTGGGT
AACGGGCCGGTGTTGATCAGTATCGAAAAACAGGTCGAATATATTGTTCAGGTACTGGGGAAAAT
GCAGAAGGAGAATCTACAGTCATTTGAAGTCCGGCGGACGGCAACAGACTCGTTTAACCAATGGA
AGGATGCATTCATGCAAAACACGGTGTGGACGAGTGGTTGTCGCAGCTGGTATCAGAATGGCTCG
AAAGGGAACCAGATCGTGGCTCTCTGGCCTGGATCCACGTTGCACTATTTGGAGGCGATTCAGCAT
CCACGATACGAGGACTACATCTGGACCAGTCCACCTGGTGTCAATCCATGGGCCTTTCTAGGCAAC
GGGCAGAGTACGGCCGAAACCCGTCCCGGAGGCGACACGAGTTGGTATCTGCGTTCGAAAGATGA
TTCATTTATAGATCCATGTCTGAGACAGCTTTAG
SEQ ID NO 16: Aspersillus wentii AspWeBVMO wt (OJJ34587.1)
MTKDNTTSFPSHAIYEPRRTLKVLVIGAGASGLLLAYKLQRHFDCVEITVFEKNPAVSGTWFENRYPGC
ACDVPSHCYTWSFEPNPNWSANYAGADEIRQYFVDFCHRHDLQKYIHLEHEVVHAAWKSETGHWEV
QVRDIQHNSHTQHTAHILINATGILNQWKWPSIPGLQSFQGDLLHSAAWDSSVNLEDKTVAVIGNGSSG
IQIVPAILPQVRKLVHFTRQAAWVAPPVNEEYQEYSPEQIERFRSDPTYLLGVRRQIEARMNGSFLKFIQ
GSDMQRRAHEYVMLHMMKRLDGDASLAETLVPTFPFGCRRPTPGTGYLEALKDSKVETITGARIANV
TGNQVVLENGTSYTVDAIVCATGFDTSYKPRFPLVGRDSTTLSEAWKDEVSAYLGLTVPGFPNYFSILG
PNCPVGNGPVLISIEKQVEYIVQVLGKMQKENLQSFEVRRTATDSFNQWKDAFMQNTVWTSGCRSWY
QNGSKGNQIVALWPGSTLHYLEAIQHPRYEDYIWTSPPGVNPWAFLGNGQSTAETRPGGDTSWYLRSK
DDSFIDPCLRQL*
SEQ ID NO 17: Aspersillus wentii AspWeBVMO E. coli optimized
ATGACCAAAGATAACACCACGTCCTTTCCGAGCCACGCCATTTACGAGCCGCGCCGTACCCTGAAA
GTCCTGGTGATCGGTGCTGGCGCGAGCGGTTTGTTGCTGGCATATAAGCTGCAGCGCCACTTCGAT
TGCGTTGAGATTACCGTATTCGAGAAGAATCCGGCAGTCAGCGGCACCTGGTTTGAGAATCGTTAC
CCTGGTTGTGCATGTGACGTGCCGAGCCATTGCTACACCTGGTCGTTCGAGCCAAACCCGAATTGG
AGCGCAAACTACGCGGGTGCGGATGAAATTCGCCAGTATTTCGTTGATTTCTGTCACCGTCATGAT
CTGCAGAAGTACATCCATCTGGAGCACGAAGTCGTTCATGCGGCATGGAAATCGGAGACTGGTCA
CTGGGAAGTGCAAGTCCGTGACATCCAGCACAACAGCCATACCCAGCACACGGCGCACATTTTGA
TCAACGCAACGGGTATCCTGAATCAATGGAAATGGCCGAGCATTCCGGGCCTGCAGAGCTTTCAG
GGTGATCTGCTGCATAGCGCAGCGTGGGACAGCTCCGTCAACTTAGAGGATAAGACCGTCGCGGT
GATCGGTAATGGCAGCAGCGGTATCCAGATTGTGCCGGCCATCCTGCCGCAAGTGCGCAAACTGG
TTCACTTTACGCGTCAAGCGGCATGGGTGGCACCGCCGGTGAACGAAGAGTACCAAGAGTACAGC
CCGGAGCAAATTGAGCGTTTCCGTAGCGACCCGACCTACCTGTTGGGCGTCCGCCGTCAAATTGAA
GCCCGTATGAACGGCAGCTTTCTGAAGTTTATTCAGGGCAGCGACATGCAGCGCAGAGCGCACGA
ATACGTTATGCTGCACATGATGAAGCGTCTGGACGGTGATGCGAGCCTTGCTGAGACTCTGGTGCC
GACGTTTCCGTTCGGCTGCCGTCGTCCGACCCCGGGCACCGGTTATCTGGAAGCGCTGAAAGACTC
TAAAGTTGAAACGATCACGGGTGCCCGTATCGCAAATGTTACGGGCAACCAAGTTGTCCTGGAGA
ACGGTACTAGCTATACGGTCGATGCTATTGTCTGTGCTACCGGTTTCGACACCAGCTATAAGCCGC
GTTTCCCGCTGGTTGGCCGCGACTCTACCACCCTGAGCGAAGCCTGGAAAGACGAAGTGTCTGCGT
ACCTGGGTCTGACCGTTCCGGGTTTTCCGAACTATTTCAGCATCCTGGGTCCTAATTGCCCGGTCGG
TAATGGTCCGGTTTTGATCAGCATCGAGAAACAAGTGGAGTATATCGTGCAAGTTCTGGGTAAGAT
GCAGAAAGAAAACTTGCAGTCCTTCGAAGTTCGCCGTACCGCCACCGACAGCTTCAATCAGTGGA
AAGATGCGTTCATGCAAAACACGGTGTGGACCTCAGGTTGCCGTTCTTGGTATCAGAATGGCAGCA
AGGGCAACCAAATTGTCGCGCTGTGGCCGGGTTCCACGCTGCACTACCTGGAAGCGATTCAACATC
CTCGCTACGAAGATTATATCTGGACGAGCCCACCGGGTGTTAATCCGTGGGCGTTTCTGGGCAATG
GCCAGAGCACCGCGGAAACCCGTCCGGGTGGCGACACTTCCTGGTATCTCCGCTCCAAAGATGAC
AGCTTTATTGACCCATGCCTGCGTCAGCTGTAA
SEQ ID NO 18: Aspergillus wentii AspWeBVMO Yeast optimized
ATGACTAAGGACAACACTACTTCTTTCCCATCTCACGCTATCTACGAACCAAGAAGAACTTTGAAG
GTTTTGGTTATCGGTGCTGGTGCTTCTGGTTTGTTGTTGGCTTACAAGTTGCAAAGACACTTCGACT
GTGTTGAAATCACTGTTTTCGAAAAGAACCCAGCTGTTTCTGGTACTTGGTTCGAAAACAGATACC
CAGGTTGTGCTTGTGACGTTCCATCTCACTGTTACACTTGGTCTTTCGAACCAAACCCAAACTGGTC
TGCTAACTACGCTGGTGCTGACGAAATCAGACAATACTTCGTTGACTTCTGTCACAGACACGACTT
GCAAAAGTACATCCACTTGGAACACGAAGTTGTTCACGCTGCTTGGAAGTCTGAAACTGGTCACTG
GGAAGTTCAAGTTAGAGACATCCAACACAACTCTCACACTCAACACACTGCTCACATCTTGATCAA
CGCTACTGGTATCTTGAACCAATGGAAGTGGCCATCTATCCCAGGTTTGCAATCTTTCCAAGGTGA
CTTGTTGCACTCTGCTGCTTGGGACTCTTCTGTTAACTTGGAAGACAAGACTGTTGCTGTTATCGGT
AACGGTTCTTCTGGTATCCAAATCGTTCCAGCTATCTTGCCACAAGTTAGAAAGTTGGTTCACTTCA
CTAGACAAGCTGCTTGGGTTGCTCCACCAGTTAACGAAGAATACCAAGAATACTCTCCAGAACAA
ATCGAAAGATTCAGATCTGACCCAACTTACTTGTTGGGTGTTAGAAGACAAATCGAAGCTAGAAT
GAACGGTTCTTTCTTGAAGTTCATCCAAGGTTCTGACATGCAAAGAAGAGCTCACGAATACGTTAT
GTTGCACATGATGAAGAGATTGGACGGTGACGCTTCTTTGGCTGAAACTTTGGTTCCAACTTTCCC
ATTCGGTTGTAGAAGACCAACTCCAGGTACTGGTTACTTGGAAGCTTTGAAGGACTCTAAGGTTGA
AACTATCACTGGTGCTAGAATCGCTAACGTTACTGGTAACCAAGTTGTTTTGGAAAACGGTACTTC
TTACACTGTTGACGCTATCGTTTGTGCTACTGGTTTCGACACTTCTTACAAGCCAAGATTCCCATTG
GTTGGTAGAGACTCTACTACTTTGTCTGAAGCTTGGAAGGACGAAGTTTCTGCTTACTTGGGTTTG
ACTGTTCCAGGTTTCCCAAACTACTTCTCTATCTTGGGTCCAAACTGTCCAGTTGGTAACGGTCCAG
TTTTGATCTCTATCGAAAAGCAAGTTGAATACATCGTTCAAGTTTTGGGTAAGATGCAAAAGGAAA
ACTTGCAATCTTTCGAAGTTAGAAGAACTGCTACTGACTCTTTCAACCAATGGAAGGACGCTTTCA
TGCAAAACACTGTTTGGACTTCTGGTTGTAGATCTTGGTACCAAAACGGTTCTAAGGGTAACCAAA
TCGTTGCTTTGTGGCCAGGTTCTACTTTGCACTACTTGGAAGCTATCCAACACCCAAGATACGAAG
ACTACATCTGGACTTCTCCACCAGGTGTTAACCCATGGGCTTTCTTGGGTAACGGTCAATCTACTGC
TGAAACTAGACCAGGTGGTGACACTTCTTGGTACTTGAGATCTAAGGACGACTCTTTCATCGACCC
ATGTTTGAGACAATTGTAA
SEQ ID NO 19: Hyphozyma roseoniera SCH23-EST wt
ATGCCTTCCGATCTTCCCCGACCAGCATATGACCCGGAAATAGAGCCCTTCCTCTCTATGGTCCCAT
TACCACCAACAATCAATGCGGATATCATGAAAGAATTGCGTAAAGCACCTCTGCTCAGTCAAGCG
CCTGACCTCGACGCATTACTTTCCGACAAGCCAATAACTCACCGCGAAGTCAGCATTCCAGGTCTC
AATTCCCAAGATCCACAAATCACGTTGTCAATATTCTCCAGTACATTGGAGGGTGGCCCGAAACCA
TGTATCTATTTCGTTCATGGTGGCGGTATGATCATCGGATGTCGATTCGTGGGTATTGAGGATTATC
TTCAATACGTCGAGCAGAACGACGCTGTCGTCGTGGCTGTAGAGTATCGTCTCGCTCCGGAACACC
CGGACCCAGCGCCTGTCAATGATTGTTACGCTGGACTTTTATGGACGGCAGCAAATGCTGCAGAGC
TAGGCATCGATCTGGAGAGACTGTTGATCTGTGGCGCTTCTGCTGGTGGTGGTCTTTCTGCTGGAG
TGGCATTGATGGCACGAGACAAGAAAGGTCCAAAATTGGTAGGACAATTGTTATGCTATCCAATG
CTCGACGATAGGAATGATTCACTCTCAAGTCAGCAGTACGTGGATGAAGGTGTTTGGAGTCGTGGT
AGCAATGCATTTGGCTGGAAGCAATTGCTTGGAGACAGGGCGGGCAAAGAGGGAGTCAGTATTTA
TGCTGCGCCGGCAAGAGCAACTGATTTGAGCGGACTGCCGAACACTTTCATCGACGTTGGCAGCG
CTGAAGTCTTCAGGGATGAGGACATCGCTTATGCCTCGAGGTTATGGGCTGTTGGTGTCCAAGCGG
AACTTCATGTGTGGCCGGGTGGATATCATGCTGCGGAGAACATGGCACCTGGGACTGATTACTCTA
AGAAGGTGAAAGCGACTCGCTTGGCATGGATGAAGAGAGTCTTCATGAAAGCCCCAAAGTCGACG
ACAGAGTCGTTGCCTGCTCCAACAGTGGATGAAGCTGTTGGCACAATATGA
SEQ ID NO 20: Hyphozyma roseoniera SCH23-EST wt
MPSDLPRPAYDPEIEPFLSMVPLPPTINADIMKELRKAPLLSQAPDLDALLSDKPITHREVSIPGLNSQDP
QITLSIFSSTLEGGPKPCIYFVHGGGMIIGCRFVGIEDYLQYVEQNDAVVVAVEYRLAPEHPDPAPVNDC
YAGLLWTAANAAELGIDLERLLICGASAGGGLSAGVALMARDKKGPKLVGQLLCYPMLDDRNDSLSS
QQYVDEGVWSRGSNAFGWKQLLGDRAGKEGVSIYAAPARATDLSGLPNTFIDVGSAEVFRDEDIAYA
SRLWAVGVQAELHVWPGGYHAAENMAPGTDYSKKVKATRLAWMKRVFMKAPKSTTESLPAPTVDE
AVGTI
SEQ ID NO 21: Hyphozyma roseonisra SCH23-EST E. coli optimized
ATGCCATCGGATCTGCCGCGCCCAGCCTACGACCCTGAAATCGAACCGTTCTTGAGCATGGTTCCG
CTGCCTCCGACCATTAACGCGGACATTATGAAAGAACTGCGTAAGGCCCCACTGCTGAGCCAGGC
TCCGGATCTGGATGCCCTGCTGAGCGACAAGCCGATTACTCACCGTGAGGTGTCCATCCCGGGTCT
GAACAGCCAGGACCCGCAGATTACCCTGAGCATCTTTAGCTCTACCCTGGAGGGTGGCCCGAAGC
CGTGTATCTACTTCGTGCACGGTGGCGGCATGATTATTGGCTGTCGCTTCGTCGGTATTGAGGACT
ACTTGCAATACGTGGAACAGAATGACGCGGTCGTTGTGGCCGTTGAGTATCGTCTGGCACCGGAA
CATCCGGACCCGGCACCGGTGAATGACTGCTACGCGGGTCTGCTGTGGACCGCTGCGAACGCGGC
AGAACTGGGCATCGATTTGGAGCGTCTGCTGATCTGCGGCGCTTCTGCGGGTGGCGGTCTGTCAGC
GGGTGTGGCGCTGATGGCACGCGACAAAAAGGGTCCGAAACTGGTCGGTCAGCTGCTGTGCTATC
CGATGCTCGACGATCGTAACGATAGCTTGAGCAGCCAGCAATACGTAGATGAGGGTGTTTGGAGC
CGTGGTAGCAATGCGTTTGGTTGGAAGCAACTGCTGGGTGATCGTGCCGGCAAAGAGGGCGTGTC
CATTTACGCGGCACCGGCTCGCGCAACCGACCTGTCTGGCTTGCCTAACACGTTTATCGACGTTGG
TTCCGCCGAGGTTTTCCGTGATGAAGATATCGCGTATGCGAGCCGCTTATGGGCAGTCGGTGTTCA
AGCGGAGCTGCATGTCTGGCCGGGTGGTTATCACGCTGCGGAGAATATGGCACCGGGCACCGATT
ATAGCAAAAAAGTCAAGGCGACGCGTCTGGCATGGATGAAACGCGTCTTTATGAAGGCCCCGAAA
AGCACCACGGAGAGCCTGCCGGCACCGACGGTTGACGAAGCGGTGGGCACGATCTAA
SEQ ID NO 22: Hyphozyma roseonisra SCH23-EST Yeast optimized
ATGCCATCTGACTTGCCAAGACCAGCTTACGACCCAGAAATCGAACCATTCTTGTCTATGGTTCCA
TTGCCACCAACTATCAACGCTGACATCATGAAGGAATTGAGAAAGGCTCCATTGTTGTCTCAAGCT
CCAGACTTGGACGCTTTGTTGTCTGACAAGCCAATCACTCACAGAGAAGTTTCTATCCCAGGTTTG
AACTCTCAAGACCCACAAATCACTTTGTCTATCTTCTCTTCTACTTTGGAAGGTGGTCCAAAGCCAT
GTATCTACTTCGTTCACGGTGGTGGTATGATCATCGGTTGTAGATTCGTTGGTATCGAAGACTACTT
GCAATACGTTGAACAAAACGACGCTGTTGTTGTTGCTGTTGAATACAGATTGGCTCCAGAACACCC
AGACCCAGCTCCAGTTAACGACTGTTACGCTGGTTTGTTGTGGACTGCTGCTAACGCTGCTGAATT
GGGTATCGACTTGGAAAGATTGTTGATCTGTGGTGCTTCTGCTGGTGGTGGTTTGTCTGCTGGTGTT
GCTTTGATGGCTAGAGACAAGAAGGGTCCAAAGTTGGTTGGTCAATTGTTGTGTTACCCAATGTTG
GACGACAGAAACGACTCTTTGTCTTCTCAACAATACGTTGACGAAGGTGTTTGGTCTAGAGGTTCT
AACGCTTTCGGTTGGAAGCAATTGTTGGGTGACAGAGCTGGTAAGGAAGGTGTTTCTATCTACGCT
GCTCCAGCTAGAGCTACTGACTTGTCTGGTTTGCCAAACACTTTCATCGACGTTGGTTCTGCTGAAG
TTTTCAGAGACGAAGACATCGCTTACGCTTCTAGATTGTGGGCTGTTGGTGTTCAAGCTGAATTGC
ACGTTTGGCCAGGTGGTTACCACGCTGCTGAAAACATGGCTCCAGGTACTGACTACTCTAAGAAGG
TTAAGGCTACTAGATTGGCTTGGATGAAGAGAGTTTTCATGAAGGCTCCAAAGTCTACTACTGAAT
CTTTGCCAGCTCCAACTGTTGACGAAGCTGTTGGTACTATCTAA
SEQ ID NO 23: Filobasidium magnum SCH24-EST wt
ATGACTCATAGCCCTCCACTCGATGCCGAACTTTCGCTACTCCGATATGCTCCTGCTGTTCCCGTGG
GATGGCAGTTGGGACGAAAACTCTTGCGGATGAACACACTCATGACGCGCCCTATGGAGGGTGTC
ATGCGAGATGATGTGGTCATACCAAATCTTGATGGTACTGCCAACATCAGACTGTTCATTTGTCGC
CCTCAAGACCCTACTGAGACTATGCCGGTGATACTTTGGTTACACGGAGGCGGTATGGTCGCAGGT
CATTACAAACAAGACTCCGGGTTCATGGACATCTGGGCCAAGCGCCTAGGAGCCTTTGTGGTTTCG
GTCGATTATCGTCTGGCTCCCGAGGCCAAGGCTCCAGCCGCTCTAGACGATTGCATCGCTGCTTGG
CAATGGATCACCACGCAGACCGCTCGAGGCATCGACACTACCCGCATGGCGGTGGGTGGTGCGAG
CGCAGGAGGAGGCCTGGCGGCCAGTACCGTTCAGCGACTTGTCGATCTCGGAGGAGTGAAACCTG
TCTTTCAATTGCTCATCTATCCCATGTTGGACGACAGGACGGTGGTCAGATTTGATCCCGACCGAA
GATATTACATGTGGACACCGGATTGTAATCGATATGGCTGGACCTCGTACCTCGGAGTCCCTCCAG
GGAGCGCTGAGGTGCCTCCCTATGCGTCGGCGGCACGTCGACCGGATCTATCAGGTCTACCTCCCA
CCTGGATCGGTGTTGGGTCACTGGATCTCTTTCACGACGAGGACATGGATTATGCGCGCAGGTTAC
GTGAGAGCGGAGTTCCGGTTGAGGAATATGTCGCTGTCGGAGCGCCTCATGCCTTCGACACGATAT
ATGGAAAGGCGAAGGTCACCTTGGATTTCTGGGACTCGCATTTCAACGCCCTTCGAAGGGCTTTGT
GTCTCGACTGA
SEQ ID NO 24: Filobasidium magnum SCH24-EST wt
MTHSPPLDAELSLLRYAPAVPVGWQLGRKLLRMNTLMTRPMEGVMRDDVVIPNLDGTANIRLFICRPQ
DPTETMPVILWLHGGGMVAGHYKQDSGFMDIWAKRLGAFVVSVDYRLAPEAKAPAALDDCIAAWQ
WITTQTARGIDTTRMAVGGASAGGGLAASTVQRLVDLGGVKPVFQLLIYPMLDDRTVVRFDPDRRYY
MWTPDCNRYGWTSYLGVPPGSAEVPPYASAARRPDLSGLPPTWIGVGSLDLFHDEDMDYARRLRESG
VPVEEYVAVGAPHAFDTIYGKAKVTLDFWDSHFNALRRALCLD
SEQ ID NO 25: Filobasidium magnum SCH24-EST E. coli optimized
ATGACCCACTCGCCGCCACTGGATGCCGAACTGAGCTTGCTGCGCTACGCCCCTGCCGTTCCGGTG
GGTTGGCAGCTGGGTCGCAAACTGCTGCGTATGAACACCTTGATGACCCGTCCGATGGAAGGTGTC
ATGCGCGACGATGTGGTTATTCCGAATCTGGACGGCACGGCTAACATCCGTCTGTTTATCTGTCGT
CCGCAAGACCCGACCGAGACTATGCCGGTTATCCTGTGGCTGCACGGTGGCGGCATGGTCGCAGG
CCACTACAAACAAGACAGCGGTTTCATGGACATTTGGGCGAAGCGCCTGGGTGCGTTTGTTGTTAG
CGTTGATTATCGCCTGGCGCCTGAGGCTAAGGCACCGGCAGCGCTCGATGACTGCATCGCGGCGTG
GCAGTGGATTACCACCCAGACCGCGCGTGGTATTGACACCACTCGTATGGCAGTGGGTGGTGCGA
GCGCGGGTGGCGGTCTGGCGGCAAGCACGGTTCAGCGTCTTGTCGATCTGGGCGGTGTGAAACCG
GTCTTTCAACTGCTGATCTATCCGATGCTGGACGATCGTACCGTGGTGCGCTTCGACCCGGATCGT
CGTTATTACATGTGGACGCCGGACTGCAACAGATACGGCTGGACCAGCTACCTGGGCGTGCCACC
GGGTAGCGCAGAGGTCCCGCCGTATGCCTCCGCGGCTCGTCGTCCGGATCTGTCCGGCCTGCCGCC
GACGTGGATCGGTGTCGGCTCTCTGGATCTGTTCCATGACGAAGATATGGATTACGCACGTCGTTT
GCGCGAGAGCGGTGTGCCGGTCGAAGAGTATGTTGCTGTGGGTGCCCCGCATGCGTTCGACACGA
TTTACGGCAAGGCCAAAGTTACGCTGGACTTTTGGGATAGCCACTTCAATGCGCTGCGCCGTGCGT
TGTGTTTAGACTAA
SEQ ID NO 26: Filobasidium magnum SCH24-EST Yeast optimized
ATGACTCACTCTCCACCATTGGACGCTGAATTGTCTTTGTTGAGATACGCTCCAGCTGTTCCAGTTG
GTTGGCAATTGGGTAGAAAGTTGTTGAGAATGAACACTTTGATGACTAGACCAATGGAAGGTGTT
ATGAGAGACGACGTTGTTATCCCAAACTTGGACGGTACTGCTAACATCAGATTGTTCATCTGTAGA
CCACAAGACCCAACTGAAACTATGCCAGTTATCTTGTGGTTGCACGGTGGTGGTATGGTTGCTGGT
CACTACAAGCAAGACTCTGGTTTCATGGACATCTGGGCTAAGAGATTGGGTGCTTTCGTTGTTTCT
GTTGACTACAGATTGGCTCCAGAAGCTAAGGCTCCAGCTGCTTTGGACGACTGTATCGCTGCTTGG
CAATGGATCACTACTCAAACTGCTAGAGGTATCGACACTACTAGAATGGCTGTTGGTGGTGCTTCT
GCTGGTGGTGGTTTGGCTGCTTCTACTGTTCAAAGATTGGTTGACTTGGGTGGTGTTAAGCCAGTTT
TCCAATTGTTGATCTACCCAATGTTGGACGACAGAACTGTTGTTAGATTCGACCCAGACAGAAGAT
ACTACATGTGGACTCCAGACTGTAACAGATACGGTTGGACTTCTTACTTGGGTGTTCCACCAGGTT
CTGCTGAAGTTCCACCATACGCTTCTGCTGCTAGAAGACCAGACTTGTCTGGTTTGCCACCAACTT
GGATCGGTGTTGGTTCTTTGGACTTGTTCCACGACGAAGACATGGACTACGCTAGAAGATTGAGAG
AATCTGGTGTTCCAGTTGAAGAATACGTTGCTGTTGGTGCTCCACACGCTTTCGACACTATCTACG
GTAAGGCTAAGGTTACTTTGGACTTCTGGGACTCTCACTTCAACGCTTTGAGAAGAGCTTTGTGTTT
GGACTAA
SEQ ID NO 27: Papiliotrema laurentii SCH25-EST wt
ATGCCTTCCAATCTCCCCCGACCAGCATATGACCCGGAAATAGAGCCATTCCTCTCTATGGTCCCA
TTACCACCAACAATCAATGCGGATATCATGAGAGAACTGCGTAAAGCGCCTCTACTCAGTCAAGC
GCCTGACCTCGACGCATTACTTTCCGGCAAACCAATAACTCACCGCGAAGTCAGCATTCCAGGTCT
CAATTCTTCAGATCCACAAATCACGTTGTCGATATTCTCCAGTACATTGACGAGCGGTCCAAAACC
ATGTATTTATTTCGTTCATGGTGGCGGTATGATCATCGGATGTCGATTCGTGGGTATTGAGGATTAT
CTTCAGTACGTCGAGCAGAATGACGCTGTCGTCGTGGCTGTGGAATATCGTCTTGCGCCGGAAAAT
CCAGATCCAGCGCCTGTCAATGATTGTTACGCTGGACTTCTATGGACCGCAGCAAATGCTGCAGAA
CTGGGCATTGATCTGGAGAGACTGTTGATCTGTGGCGCTTCTGCCGGTGGTGGTCTTTCTGCTGGA
GTGGCTTTGATGGCGCGAGACAAGAAAGGTCCGAAACTGGTAGGACAATTGTTATGTTATCCGAT
GCTCGACGATAGGAATGATTCCCTTTCAAGTCAGCAGTACGTCGATGAAGGTGTTTGGAGTCGTGG
TAGCAATGCATTCGGGTGGAAGCAATTGCTTGGAGACAGGGCAGGCAAAGAAGGTGTCAGCATCT
ATGCTGCACCGGCGAGGGCAACTGATTTGAGCGGACTGCCGAACACTTTCATCGACGTTGGCAGC
GCTGAAGTCTTCAGGGATGAGGACATCGCTTATGCCTCGAGGTTATGGGCTGTCGGTGTCCAAGCA
GAACTTCATGTGTGGCCGGGTGGATATCATGCTGCGGAAAACATGGCACCGGGGACTGACTACTC
TAACAAGGTGAAAGCTGCCCGCTTGGCATGGATGAAGAGAGTCTTCATGAAAGCCCCAAAGTCGA
CGACAGAGTCGTTACCTGCTCCAACAGTGGATGAAGCTGTTGGCACAATATGA
SEQ ID NO 28: Papiliotrema laurentii SCH25-EST wt
MPSNLPRPAYDPEIEPFLSMVPLPPTINADIMRELRKAPLLSQAPDLDALLSGKPITHREVSIPGLNSSDPQ
ITLSIFSSTLTSGPKPCIYFVHGGGMIIGCRFVGIEDYLQYVEQNDAVVVAVEYRLAPENPDPAPVNDCY
AGLLWTAANAAELGIDLERLLICGASAGGGLSAGVALMARDKKGPKLVGQLLCYPMLDDRNDSLSSQ
QYVDEGVWSRGSNAFGWKQLLGDRAGKEGVSIYAAPARATDLSGLPNTFIDVGSAEVFRDEDIAYAS
RLWAVGVQAELHVWPGGYHAAENMAPGTDYSNKVKAARLAWMKRVFMKAPKSTTESLPAPTVDEA
VGTI
SEQ ID NO 29: Papiliotrema laurentii SCH25-EST E. coli optimized
ATGCCAAGCAACTTGCCGCGCCCAGCCTACGATCCGGAAATTGAGCCTTTTCTGTCTATGGTCCCG
CTGCCGCCGACCATCAACGCGGACATTATGCGTGAGCTGCGTAAAGCCCCGCTGCTGAGCCAGGC
ACCGGACCTCGACGCACTGCTGAGCGGCAAGCCGATCACTCACCGTGAAGTCAGCATTCCGGGTC
TGAACAGCAGCGACCCGCAAATCACCCTGAGCATTTTCTCCAGCACGTTGACCAGCGGTCCGAAA
CCGTGCATCTATTTTGTGCACGGTGGCGGTATGATTATTGGTTGTCGCTTCGTCGGCATTGAAGATT
ATCTGCAATATGTTGAGCAAAATGACGCGGTGGTTGTGGCGGTTGAGTATCGTCTGGCCCCTGAAA
ATCCGGACCCGGCACCGGTTAATGATTGCTACGCGGGTCTGCTGTGGACCGCAGCGAACGCAGCG
GAGCTGGGTATCGATTTGGAACGCCTGCTGATCTGTGGCGCGAGCGCTGGCGGTGGTCTGAGCGC
GGGTGTGGCGCTGATGGCTCGCGACAAAAAGGGTCCAAAACTGGTCGGTCAGCTGTTGTGCTACC
CGATGCTGGACGATCGTAACGACAGCTTGAGCTCTCAACAGTACGTCGATGAGGGTGTTTGGAGC
CGTGGCAGCAATGCTTTCGGCTGGAAACAGCTGCTGGGCGATCGTGCGGGTAAGGAAGGCGTGTC
GATCTATGCCGCTCCGGCACGCGCAACCGATCTGTCTGGCCTGCCGAACACGTTCATCGATGTCGG
TAGCGCTGAGGTGTTTCGTGACGAAGATATCGCGTACGCCTCACGTCTGTGGGCCGTCGGTGTGCA
GGCCGAGCTGCATGTTTGGCCGGGTGGCTACCATGCAGCCGAGAATATGGCGCCTGGCACCGACT
ACTCCAATAAAGTGAAGGCAGCGCGCCTGGCGTGGATGAAGCGTGTGTTTATGAAAGCGCCGAAG
TCCACGACCGAGAGCCTGCCGGCACCGACCGTTGACGAAGCGGTTGGTACGATTTAA
SEQ ID NO 30: Bensingtonia ciliata SCH46-EST wt
ATGCCTTCCAATCTCCCTCGACCAGCATATGACCCGGAAATAGAGCCATTCCTCTCTATGGTCCCA
TTACCACCAACAATCAATGCGGATATCATGAGAGAACTGCGTAAAGCACCTCTACTCAGTCAAGC
GCCTGACCTCGACGCATTACTTTCCGGCAGACCGATAACTCACCGCGAAGTCAGCATTCCAGGTCT
CAATTCCCAGGATCCACAAATCACGTTGTCAATATTCTCCAGTACATTGACGAGCGGTCCAAAACC
ATGTATTTATTTCGTTCATGGTGGCGGTATGATCATCGGATGTCGATTCGTGGGTATTGAGGATTAT
CTTCAATACGTCGAGCAGAACGACGCTGTCGTTGTGGCTGTGGAATATCGTCTTGCTCCGGAAAAC
CCGGACCCAGCGCCTGTTAATGATTGTTACGCCGGACTTTTATGGACCGCAGCGAATGCTGCAGAG
CTAGGCATCGATCTGGAGAGACTGTTGATCTGTGGCGCTTCTGCCGGTGGTGGTCTTTCTGCTGGA
GTGGCATTGATGGCACGAGACAAGAAAGGTCCAAAATTGGTAGGACAATTGTTATGCTATCCAAT
GCTCGACGATAGGAATGATTCACTCTCAAGTCAGCAGTACGTGGATGAAGGTGTTTGGAGTCGTG
GTAGCAATGCATTTGGCTGGAAGCAATTGCTTGGAGACAGGGCGGGCAAAGAGGGAGTCAGTATT
TATGCTGCGCCGGCAAGAGCAACTGATTTGAGCGGACTGCCGAACACTTTCATCGACGTTGGCAGC
GCTGAGGTCTTCAGGGATGAGGACATCGCTTATGCCTCGAGGTTATGGGCTGTCGGTGTCCAAGCA
GAACTTCATGTGTGGCCCGGTGGATATCATGCTGCGGAGAACATGGCACCGGGGACTGACTACTCT
AAGAAGGTGAAAGCTGCGCGCTTGGCATGGATGAAGAGAGTCTTCCTGAAAGCCCCAAAGCCGAC
GACTGAGTCGTTGCCTGCTCCAACAGTGGATGAAGCTGTTGGCACAATATGA
SEQ ID NO 31: Bensingtonia ciliata SCH46-EST wt
MPSNLPRPAYDPEIEPFLSMVPLPPTINADIMRELRKAPLLSQAPDLDALLSGRPITHREVSIPGLNSQDP
QITLSIFSSTLTSGPKPCIYFVHGGGMIIGCRFVGIEDYLQYVEQNDAVVVAVEYRLAPENPDPAPVNDC
YAGLLWTAANAAELGIDLERLLICGASAGGGLSAGVALMARDKKGPKLVGQLLCYPMLDDRNDSLSS
QQYVDEGVWSRGSNAFGWKQLLGDRAGKEGVSIYAAPARATDLSGLPNTFIDVGSAEVFRDEDIAYA
SRLWAVGVQAELHVWPGGYHAAENMAPGTDYSKKVKAARLAWMKRVFLKAPKPTTESLPAPTVDE
AVGTI
SEQ ID NO 32: Bensingtonia ciliata SCH46-EST E. coli optimized
ATGCCATCGAATCTGCCGCGTCCAGCCTACGACCCTGAAATTGAACCTTTCTTGAGCATGGTGCCG
CTGCCGCCGACGATTAACGCTGATATCATGCGTGAGCTGCGCAAGGCACCGCTGCTGAGCCAAGC
GCCGGACCTGGATGCGCTGTTGAGCGGTCGCCCGATCACCCACCGCGAAGTCAGCATCCCGGGTCT
GAACTCTCAGGACCCGCAGATCACCTTGTCAATCTTTAGCAGCACCTTGACTTCCGGTCCGAAGCC
GTGCATTTATTTTGTCCACGGTGGTGGCATGATTATCGGCTGTCGTTTCGTTGGTATTGAAGATTAC
TTACAATATGTGGAACAAAATGATGCAGTGGTTGTGGCAGTGGAGTACCGCCTGGCGCCTGAGAA
CCCGGACCCAGCGCCGGTGAACGACTGCTACGCGGGTCTGTTGTGGACGGCAGCTAACGCAGCAG
AGCTGGGTATCGATCTGGAGCGCCTGCTGATCTGCGGTGCGAGCGCGGGTGGCGGCCTGTCCGCTG
GCGTTGCGCTGATGGCCCGTGACAAAAAGGGTCCGAAACTGGTTGGCCAGCTGCTGTGTTATCCGA
TGCTGGACGACCGTAATGACAGCCTGAGCAGCCAGCAATACGTGGATGAGGGCGTCTGGAGCCGT
GGTAGCAATGCGTTCGGTTGGAAGCAACTGCTGGGCGATCGTGCCGGCAAAGAGGGCGTTAGCAT
CTATGCGGCACCGGCGCGTGCCACGGATCTGTCTGGTCTGCCGAACACCTTCATTGACGTTGGTAG
CGCTGAAGTTTTTCGCGATGAAGATATTGCGTACGCGAGCCGTCTGTGGGCAGTCGGCGTCCAGGC
AGAGCTCCATGTCTGGCCGGGTGGCTATCATGCGGCCGAGAATATGGCACCGGGTACGGACTACA
GCAAAAAAGTTAAAGCTGCGCGTCTGGCCTGGATGAAGCGTGTTTTCCTGAAAGCGCCGAAGCCG
ACCACCGAGTCCCTGCCGGCACCGACCGTGGATGAAGCCGTGGGCACCATTTAA
SEQ ID NO 33: Rhodococcus erythropolis SCH94-3944 wt
ATGAATCTCAACGAAGCCCGAACTGCTTTCGCCCGGCTCCGTGCAGCGGAAAATGGTTTATCACCA
GCAGAACTCGACGAAGTGTGGGCCGCGCTGGAAACCGTCGCCGCTGAAGAAATCCTCGGTGAGTG
GAAAGGTGACGACTTCGCCACCGGTCATCGTCTGCACGAAAAGCTGTCCGCGAGCCGCTGGTACG
GCAAGACTTTCAATTCCGTCGAGGATGCCAAGCCGTTGATCTGCCGAGACGAAGACGGAAATCTC
TATTCCGACGTCAAGAGCGGCAATGGCGAGGCAAGTCTGTGGAACATCGAGTTTCGTGGTGAAGT
GACCGCGACCATGGTCTACGACGGCGCGCCGATTTTCGACCACTTCAAGAAAGTCGACGATTCGA
CGCTCATGGGCATCATGAACGGAAAGTCGGCGTTGGTCCTCGACGGCGGGCAGCACTACTACTTCC
TGCTCGAGCGAGCGTGA
SEQ ID NO 34: Rhodococcus erythropolis SCH94-3944 wt (WP_042451379)
MNTNEARTAFARIRAAENGTSPARIDEVWAATRTVAABETTGEWKGDDFATGHRIHEKISASRWYG
KTFNSVEDAKPLICRDEDGNLYSDVKSGNGEASLWNIEFRGEVTATMVYDGAPIFDHFKKVDDSTLMG
IMNGKSALVLDGGQHYYFLLERA
SEQ ID NO 35: Rhodococcus erythropolis SCH94-3944 E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 36: Rhodococcus erythropolis SCH94-3944 Yeast optimized
ATGAACTTGGACGAAGCTAGAACTGCTTTCGCTAGATTGAGAGCTGCTGAATCTGGTGTTTCTCCA
GCTGAATTGGACGAAGTTTGGGCTGCTTTGGAAACTGTTGCTGCTGAAGAAATCTTGGGTGAATGG
AAGGGTGACGACTTCGCTACTGGTCACAGATTGCACGAAAAGTTGTTCGCTTCTAGATGGTACGGT
AAGACTTTCAACTCTGTTGAAGACGCTAAGCCATTGATCTGTAGAGACGAAGACGGTAACTTGTAC
TCTGACGTTAAGTCTGGTAACGGTGAAGCTTCTTTGTGGAACATCGAATTCAGAGGTGAAGTTACT
GCTACTATGGTTTACGACGGTGCTCCAATCTTCGACCACTTCAAGAAGGTTGACGACTCTACTTTG
ATGGGTATCATGAACGGTAAGTCTGCTTTGGTTTTGGACGGTGGTCAACACTACTACTTCTTGTTGG
AAAGAGCTTAA
SEQ ID NO 37: Rhodococcus rhodochrous SCH80-05241 wt
ATGAATCTCGACGAAGCCCGAACTGCTTTCGCCCGGCTCCGTGCTGCGGAAAGTGGTGTATCACCA
GCAGAACTCGACGAAGTGTGGGCCGCGCTGGAAACCGTCGCCGCCGAAGAAATCCTCGGCGAGTG
GAAGGGTGACGACTTCGCCACCGGTCACCGTCTTCACGAAAAGCTGTTCGCGAGCCGTTGGTACG
GCAAGACCTTCAACTCGGTCGAGGACGCCAAGCCGTTGATCTGCCGAGACGAAGACGGCAACCTC
TACTCCGACGTCAAGAGCGGCAATGGCGAGGCAAGTCTGTGGAACATCGAGTTTCGTGGCGAAGT
CACGGCGACGATGGTCTACGACGGCGCGCCGATCTTCGACCACTTCAAGAAGGTCGACGATTCGA
CGCTCATGGGCATCATGAACGGAAAATCGGCGTTGGTTCTCGACGGCGGACAGCACTACTACTTCC
TGCTCGAGCGAGCGTGA
SEQ ID NO 38: Rhodococcus rhodochrous SCH80-05241 wt
MNLDEARTAFARLRAAESGVSPAELDEVWAALETVAAEEILGEWKGDDFATGHRLHEKLFASRWYG
KTFNSVEDAKPLICRDEDGNLYSDVKSGNGEASLWNIEFRGEVTATMVYDGAPIFDHFKKVDDSTLMG
IMNGKSALVLDGGQHYYFLLERA*
SEQ ID NO 39: Rhodococcus rhodochrous SCH80-05241 E. coli optimized
ATGAATCTGGACGAAGCCCGTACTGCTTTCGCCCGTCTGCGCGCTGCTGAATCTGGTGTTAGCCCG
GCAGAGCTGGACGAAGTGTGGGCAGCGCTGGAAACCGTTGCGGCGGAAGAAATTCTGGGTGAGTG
GAAGGGCGATGACTTCGCAACGGGCCATCGCTTGCACGAGAAATTGTTCGCGAGCCGCTGGTATG
GTAAGACCTTTAACAGCGTCGAAGATGCGAAACCGCTGATCTGCCGTGATGAAGATGGCAACCTG
TACAGCGACGTCAAGAGCGGTAATGGTGAGGCCAGCCTGTGGAATATCGAGTTTCGTGGCGAAGT
GACCGCGACGATGGTGTACGACGGTGCACCGATTTTTGATCATTTCAAAAAAGTCGATGACAGCA
CCCTGATGGGCATCATGAACGGTAAGTCCGCGCTGGTTCTGGACGGTGGCCAGCACTATTACTTTC
TGCTGGAGCGTGCGTAA
SEQ ID NO 40: Rhodococcus rhodochrous SCH80-05241 Yeast optimized
ATGAACTTGAACGAAGCTAGAACTGCTTTCGCTAGATTGAGAGCTGCTGAAAACGGTTTGTCTCCA
GCTGAATTGGACGAAGTTTGGGCTGCTTTGGAAACTGTTGCTGCTGAAGAAATCTTGGGTGAATGG
AAGGGTGACGACTTCGCTACTGGTCACAGATTGCACGAAAAGTTGTCTGCTTCTAGATGGTACGGT
AAGACTTTCAACTCTGTTGAAGACGCTAAGCCATTGATCTGTAGAGACGAAGACGGTAACTTGTAC
TCTGACGTTAAGTCTGGTAACGGTGAAGCTTCTTTGTGGAACATCGAATTCAGAGGTGAAGTTACT
GCTACTATGGTTTACGACGGTGCTCCAATCTTCGACCACTTCAAGAAGGTTGACGACTCTACTTTG
ATGGGTATCATGAACGGTAAGTCTGCTTTGGTTTTGGACGGTGGTCAACACTACTACTTCTTGTTGG
AAAGAGCTTAA
SEQ ID NO 41: Penicillium disitatum Pdigit7033 wt
ATGTCCACAAGCACCCCACAGGATCAGTTTGCTGCCCTAGTTGCAAAAAACAGCAAGTTGAATGA
AACCGACATCGAGGCTGTTTATAACAAGCTTTCAGCTCTTCCCGTCGATTTCCTCCGTGGAGAATG
GAAGGGTGGAAGCTTCGACACCGGCCACCCAGGCCACACCCAGCTTTTGGCTATGAACTGGGTTG
GAAAGACGTTCCACGATACCGAGCGCGTCGACCCTATTGTTGTGTTAAAGGATGGAAAGCGTGTA
TGCGATGAGAACTGGGGCCATGCTATCGTCCGTGAGGTTCGTTTCCGTGGTATTGTGTCAACCGCT
ATGATCTATGACAAGCACCCTATCATTGATCACTTCCGCTATGTTAATGAGAACCTCGTTGCTGGC
GCCATGGACACTAGCTCCTTCGGTGACGTTGGTACCTACTACTTCTACCTATACAAATAG
SEQ ID NO 42: Penicillium disitatum Pdigit7033 wt
MSTSTPQDQFAALVAKNSKLNETDIEAVYNKLSALPVDFLRGEWKGGSFDTGHPGHTQLLAMNWVG
KTFHDTERVDPIVVLKDGKRVCDENWGHAIVREVRFRGIVSTAMIYDKHPIIDHFRYVNENLVAGAMD
TSSFGDVGTYYFYLYK*
SEQ ID NO 43: Penicillium disitatum Pdigit7033 E. coli optimized
ATGTCCACTAGCACCCCACAAGATCAATTTGCCGCACTGGTTGCCAAAAACTCTAAACTGAATGAA
ACCGACATTGAAGCTGTCTATAACAAGTTGAGCGCGTTGCCGGTGGATTTCCTGCGTGGCGAGTGG
AAGGGCGGCAGCTTCGACACCGGTCACCCGGGTCACACGCAGCTGCTGGCAATGAATTGGGTCGG
TAAGACCTTTCATGATACCGAGCGTGTGGACCCGATCGTCGTTCTGAAGGACGGTAAACGTGTGTG
CGACGAGAATTGGGGTCACGCGATCGTTCGCGAAGTTCGCTTCCGTGGTATCGTGAGCACCGCGAT
GATCTATGATAAACACCCGATTATTGATCATTTCCGCTATGTTAACGAAAACCTGGTCGCGGGTGC
GATGGATACGTCGAGCTTTGGCGACGTGGGCACGTACTACTTTTACCTGTACAAATAA
SEQ ID NO 44: Penicillium digitatum Pdigit7033 Yeast optimized
ATGTCTACTTCTACTCCACAAGACCAATTCGCTGCTTTGGTTGCTAAGAACTCTAAGTTGAACGAA
ACTGACATCGAAGCTGTTTACAACAAGTTGTCTGCTTTGCCAGTTGACTTCTTGAGAGGTGAATGG
AAGGGTGGTTCTTTCGACACTGGTCACCCAGGTCACACTCAATTGTTGGCTATGAACTGGGTTGGT
AAGACTTTCCACGACACTGAAAGAGTTGACCCAATCGTTGTTTTGAAGGACGGTAAGAGAGTTTGT
GACGAAAACTGGGGTCACGCTATCGTTAGAGAAGTTAGATTCAGAGGTATCGTTTCTACTGCTATG
ATCTACGACAAGCACCCAATCATCGACCACTTCAGATACGTTAACGAAAACTTGGTTGCTGGTGCT
ATGGACACTTCTTCTTTCGGTGACGTTGGTACTTACTACTTCTACTTGTACAAGTAA
SEQ ID NO 45: Penicillium italicum PitalDUF4334-1 wt (JQGA01001114.1
71518-72084 (+))
ATGTCGGCCAGTGACCCCAAGGACCAGTTTGCTGCCCTAGTTGCCAAGGACGGCAAGTTGAATGA
AGACGAAATCGAGGCTGTTTACAACAAGCTTCCTGCTCTTCCCCTCGATTTCCTCCGTGGAGAATG
GAAGGGTGGAAGCTTCGACACCGGTCACCCTGGTCACACCCAACTCTTGGCAATGAAATGGGTTG
GGAAGACATTCCATTCCACCGAACGGGTTGACCCTATTGTTGTGTTAAAGGATGAAAAGCGTGTAT
GCAATGAGGACTGGGGCCATGCAGTCCTCCGTGAGATTCGTTTCCGTGGTATTGTGTCATCTGCTA
TGATCTATGACAAGCACCCTATCATCGACCACTTCCGCTATGTCAACGACAAGCTCATTGCTGGCG
CCATGGACACTAGCAGCTTCGGTGACGTTGGCACCTACTACTTCTACCTGTGCAAATAG
SEQ ID NO 46: Penicillium italicum PitalDUF4334-1 wt (KGO69886.1)
MSASDPKDQFAALVAKDGKLNEDEIEAVYNKLPALPLDFLRGEWKGGSFDTGHPGHTQLLAMKWVG
KTFHSTERVDPIVVLKDEKRVCNEDWGHAVLREIRFRGIVSSAMIYDKHPIIDHFRYVNDKLIAGAMDT
SSFGDVGTYYFYLCK*
SEQ ID NO 47: Penicillium italicum PitalDUF4334-1 E. coli optimized
ATGAGCGCTTCGGACCCAAAAGATCAATTCGCAGCATTGGTGGCAAAGGACGGTAAACTGAACGA
AGATGAAATCGAAGCCGTCTATAACAAGCTGCCTGCGCTGCCGCTGGACTTCTTGCGTGGTGAGTG
GAAGGGCGGCAGCTTTGATACCGGTCATCCGGGCCACACTCAGCTGCTGGCGATGAAATGGGTGG
GTAAAACCTTTCACAGCACCGAGCGCGTGGACCCGATCGTCGTTCTGAAAGATGAGAAGCGTGTC
TGTAATGAAGATTGGGGTCACGCCGTGCTGCGCGAGATTCGTTTTCGCGGTATCGTTTCTAGCGCG
ATGATTTATGACAAGCATCCGATTATTGACCACTTCCGTTACGTTAATGACAAGCTGATCGCGGGT
GCGATGGATACGTCCAGCTTTGGCGACGTTGGCACGTACTATTTCTACCTGTGCAAATAA
SEQ ID NO 48: Aspergillus wentii AspWe DUF4334 wt (LJSE01000065.1 (263404
to 263924))
ATGAGCTGTTGCACCGCCGAGGACCAGGCCAAACGGCTCTTCGAAGCGACCAGCCCCGTCCAACC
ATCAGCAGTCGAAGAACTCTTCAACCAACTCCAACCGATAAAGCCCTCATTCCTGATTGGCGAATG
GGACGGAAATAGCCTGGACACCGGCCATCCCGGTCTCAAGCTGCTCCAGGCGATGCGGTGGGCGG
GTAAGACATTTCGATCCGTGGATGACGCCGATCCGATTGTGACGCTGGACGATGCTGGCAATCGCA
TCTGGAAAGAGGAGTACGGTAATGCTGTGGTACGAGAAATGGCGTTTCGCGGAGTCGTTTCGGCG
GCGATGATCTACGACACCAAGCCCATCATGGACCATTTTCGATACGTGGACGAAAAGACAGTGCT
GGGTGTGATGGAAACCCCCAAGCAGGCTGGAAGCGGAACCTTTTATTTCTATCTGCAGCGTCGTGC
TTCTGTCTAA
SEQ ID NO 49: Aspergillus wentii AspWe DUF4334 wt (OJJ43591)
MSCCTAEDQAKRLFEATSPVQPSAVEELFNQLQPIKPSFLIGEWDGNSLDTGHPGLKLLQAMRWAGKT
FRSVDDADPIVTLDDAGNRIWKEEYGNAVVREMAFRGVVSAAMIYDTKPIMDHFRYVDEKTVLGVM
ETPKQAGSGTFYFYLQRRASV
SEQ ID NO 50: Aspergillus wentii AspWe DUF4334 E. coli optimized
ATGTCGTGTTGCACCGCCGAAGATCAAGCCAAACGTCTGTTCGAAGCCACTAGCCCGGTTCAACCG
AGCGCGGTCGAAGAACTGTTCAATCAGCTGCAACCGATTAAGCCTTCCTTCCTGATCGGTGAGTGG
GATGGCAACAGCCTGGATACCGGTCATCCGGGCTTGAAGCTGCTGCAGGCAATGCGCTGGGCGGG
TAAGACCTTTCGTTCTGTGGATGACGCTGACCCAATTGTTACCCTGGACGACGCGGGTAATCGTAT
TTGGAAAGAGGAATACGGTAACGCAGTGGTTCGCGAGATGGCGTTTCGTGGTGTGGTCAGCGCGG
CAATGATCTATGACACGAAGCCGATCATGGATCACTTTCGCTATGTTGACGAGAAAACGGTCCTGG
GCGTGATGGAAACGCCGAAACAGGCTGGTAGCGGCACCTTCTACTTTTACTTGCAGCGTCGTGCGA
GCGTCTAA
SEQ ID NO 51: Aspergillus wentii AspWe DUF4334 Yeast optimized
ATGTCTTGTTGTACTGCTGAAGACCAAGCTAAGAGATTGTTCGAAGCTACTTCTCCAGTTCAACCA
TCTGCTGTTGAAGAATTGTTCAACCAATTGCAACCAATCAAGCCATCTTTCTTGATCGGTGAATGG
GACGGTAACTCTTTGGACACTGGTCACCCAGGTTTGAAGTTGTTGCAAGCTATGAGATGGGCTGGT
AAGACTTTCAGATCTGTTGACGACGCTGACCCAATCGTTACTTTGGACGACGCTGGTAACAGAATC
TGGAAGGAAGAATACGGTAACGCTGTTGTTAGAGAAATGGCTTTCAGAGGTGTTGTTTCTGCTGCT
ATGATCTACGACACTAAGCCAATCATGGACCACTTCAGATACGTTGACGAAAAGACTGTTTTGGGT
GTTATGGAAACTCCAAAGCAAGCTGGTTCTGGTACTTTCTACTTCTACTTGCAAAGAAGAGCTTCT
GTTTAA
SEQ ID NO 52: Rhodococcus hoagii strain PAM2288 RhoagDUF4334-2 wt
(NZ LWTW01000167.1 18658-19134 (-))
ATGAACGTACGAGACGAGGTCGCCGCGCTGCGGGCGCGCACCGACCGGATCGATCCGCGGGAACT
CGATTCGATCTGGGACCGCTTGGCCCCGTGTCGGCCCGTGGATCTGATCGGGTACCGCTGGAGGGG
TTTCGACTTCGACACCGGACATCGCACGAGTGGGCTCCTCCGTCGAGCTCATTGGTACGGCAAGGC
ATTCGCCAGCGAGTCCGACGTGCAGCCTCTGCTGTGCCGCAGCGAGGACGGACAGCTGTTCTCCGA
CATCGGAACCGGGCACGGCGAGGCCAGCCTGTGGGAGGTCGTGTTCCGCGGGGAGGTGACCGCGA
CGATGGTCTACGACGGCATGCCGGTGTTCGACCACTTCAAGAAGGTCGACGACGACACCGTCATC
GGCGTCATGAACGGCAAGGGCACGTTGGTGTTCGACGGCGGCGAACACTTCTGGTTCGGGCTGGA
GCGAGACGTCGCACTCTGA
SEQ ID NO 53: Rhodococcus hoagii strain PAM2288 RhoagDUF4334-2 wt
(WP_005516054)
MNVRDEVAALRARTDRIDPRELDSIWDRLAPCRPVDLIGYRWRGFDFDTGHRTSGLLRRAHWYGKAF
ASESDVQPLLCRSEDGQLFSDIGTGHGEASLWEVVFRGEVTATMVYDGMPVFDHFKKVDDDTVIGVM
NGKGTLVFDGGEHFWFGLERDVAL*
SEQ ID NO 54: Rhodococcus hoagii strain PAM2288 RhoagDUF4334-2 E. coli
optimized
ATGAATGTTCGTGATGAAGTTGCAGCTCTGCGTGCCCGTACTGATAGAATCGACCCGCGTGAGCTG
GATAGCATTTGGGACCGTCTGGCACCATGTCGTCCGGTGGACCTGATCGGTTACCGTTGGCGCGGT
TTCGATTTCGACACCGGTCACCGTACCTCCGGTCTGTTGCGTCGCGCGCATTGGTATGGTAAGGCC
TTTGCGAGCGAGAGCGACGTGCAACCGTTGCTGTGCCGCTCTGAGGACGGCCAGCTGTTTAGCGAT
ATTGGCACCGGTCACGGTGAGGCGAGCCTGTGGGAAGTTGTCTTTCGCGGCGAAGTGACCGCGAC
GATGGTTTACGACGGTATGCCGGTGTTCGACCACTTCAAAAAAGTTGATGACGACACGGTGATCG
GTGTCATGAACGGCAAGGGCACGCTGGTCTTTGATGGTGGCGAGCATTTCTGGTTTGGCCTGGAAC
GCGATGTCGCGCTGTAA
SEQ ID NO 55: Rhodococcus hoagii strain N128 RhoagDUF4334-3 wt
(NZ LRQY01000021.1 163210-163686 (-))
ATGAACGTACGAGACGAGGTCGCCGCGCTGCGGGCGCGCACCGACCGGATCGATCCGCGGGAACT
CGATTCGATCTGGGACCGCTTGGCCCCGTGTCGGCCCGTGGATCTGATCGGGTACCGCTGGAGGGG
TTTCGACTTCGACACCGGACATCGCACGAGTGGGCTCCTCCGTCGAGCTCATTGGTACGGCAAGGC
ATTCGCCAGCGAGTCCGACGTGCAGCCTCTGCTGTGCCGCAGCGACGACGGACAGCTGTTCTCCGA
CATCGGAACCGGGCACGGCGAGGCCAGCCTGTGGGAGGTCGTGTTCCGCGGGGAGGTGACCGCGA
CGATGGTCTACGACGGCATGCCGGTGTTCGACCACTTCAAGAAGGTCGACGACGACACCGTCATC
GGCGTCATGAACGGCAAGGGCACGTTGGTGTTCGACGGCGGCGAACACTTCTGGTTCGGGCTGGA
GCGAGACGTCGCACTCTGA
SEQ ID NO 56: Rhodococcus hoagii strain N128 RhoagDUF4334-3 wt
(WP_013414658)
MNVRDEVAALRARTDRIDPRELDSIWDRLAPCRPVDLIGYRWRGFDFDTGHRTSGLLRRAHWYGKAF
ASESDVQPLLCRSDDGQLFSDIGTGHGEASLWEVVFRGEVTATMVYDGMPVFDHFKKVDDDTVIGVM
NGKGTLVFDGGEHFWFGLERDVAL*
SEQ ID NO 57: Rhodococcus hoagii strain N128 RhoagDUF4334-3 E. coli
optimized
ATGAATGTTCGTGATGAAGTTGCAGCTCTGCGTGCCCGTACTGATAGAATCGACCCGCGTGAGCTG
GATAGCATTTGGGACCGTCTGGCACCATGTCGTCCGGTGGACCTGATCGGTTACCGTTGGCGCGGT
TTCGATTTCGACACCGGTCACCGTACCTCCGGTCTGTTGCGTCGCGCGCATTGGTATGGTAAGGCC
TTTGCGAGCGAGAGCGACGTGCAACCGTTGCTGTGCCGCTCTGATGACGGCCAGCTGTTTAGCGAT
ATTGGCACCGGTCACGGTGAGGCGAGCCTGTGGGAAGTTGTCTTTCGCGGCGAAGTGACCGCGAC
GATGGTTTACGACGGTATGCCGGTGTTCGACCACTTCAAAAAAGTTGATGACGACACGGTGATCG
GTGTCATGAACGGCAAGGGCACGCTGGTCTTTGATGGTGGCGAGCATTTCTGGTTTGGCCTGGAAC
GCGATGTCGCGCTGTAA
SEQ ID NO 58: Rhodococcus hoagii RhoagDUF4334-4 wt (NZ BCRL01000037.1
133790-134266 (+))
ATGAACGTACGAGACGAGGTCGCCGCGCTGCGGGCGCGCACCGACCGGATCGATCCGCGGGAACT
CGATTCGATCTGGGACCGCTTGGCCCCGTGTCGGCCCGTGGATCTGATCGGGTACCGCTGGCGGGG
TTTCGACTTCGACACCGGACATCGCACGAGTGGGCTCCTCCGTCGAGCGCATTGGTACGGCAAGGC
ATTCGCCAGCGAGTCCGACGTGCAGCCTCTGCTGTGCCGCAGCGAGGACGGACAGCTGTTCTCCGA
CATCGGAACCGGGCACGGCGAGGCCAGCCTGTGGGAGGTCGTGTTCCGCGGGGAGGTGACCGCGA
CGATGGTCTACGACGGCATGCCGGTGTCCGACCACTTCAAGAAGGTCGACGACGACACCGTCATC
GGCGTCATGAACGGCAAGGGCACGTTGGTGTTCGACGGCGGCGAACACTTCTGGTTCGGGCTGGA
GCGAGACGTCGCACTCTGA
SEQ ID NO 59: Rhodococcus hoagii RhoagDUF4334-4 wt (WP_022593671)
MNVRDEVAALRARTDRIDPRELDSIWDRLAPCRPVDLIGYRWRGFDFDTGHRTSGLLRRAHWYGKAF
ASESDVQPLLCRSEDGQLFSDIGTGHGEASLWEVVFRGEVTATMVYDGMPVSDHFKKVDDDTVIGVM
NGKGTLVFDGGEHFWFGLERDVAL*
SEQ ID NO 60: Rhodococcus hoagii RhoagDUF4334-4 E. coli optimized
ATGAATGTTCGTGATGAAGTTGCAGCTCTGCGTGCCCGTACTGATAGAATCGACCCGCGTGAGCTG
GATAGCATTTGGGACCGTCTGGCACCATGTCGTCCGGTGGACCTGATCGGTTACCGTTGGCGCGGT
TTCGATTTCGACACCGGTCACCGTACCTCCGGTCTGTTGCGTCGCGCGCATTGGTATGGTAAGGCC
TTTGCGAGCGAGAGCGACGTGCAACCGTTGCTGTGCCGCTCTGAGGACGGCCAGCTGTTTAGCGAT
ATTGGCACCGGTCACGGTGAGGCGAGCCTGTGGGAAGTTGTCTTTCGCGGCGAAGTGACCGCGAC
GATGGTTTACGACGGTATGCCGGTGAGCGACCACTTCAAAAAAGTTGATGACGACACGGTGATCG
GTGTCATGAACGGCAAGGGCACGCTGGTCTTTGATGGTGGCGAGCATTTCTGGTTTGGCCTGGAAC
GCGATGTCGCGCTGTAA
SEQ ID NO 61: Cupriavidus necator CnecaDUF4334 wt (CP002879.1: 512553-
513138)
ATGCTGACAGAAATGCTGCGGAATCGAGTCTCTACAACTGCGGCGGTACTGGCCGCTTTCGATGAA
CTTGATCCATTATCGAGCGATTCGCTAGTTGGCTGCTGGAGTGGTTTTGTGATCGCTACCGGGCAC
CCCATGGACGGTCTTCTGAGCGCTGTCGGCTGGTACGGGAAAATGTTCCAAAGCGTGGATGAGGC
ATATCCGCTGATCATCCGGTCCCCGGACGCCAGTACGCTTTTTTCGATCGATCCCAGCCCTTTGCCA
CTTATAGGCTGCGCGAAGTTATCTCCCACGGATATGGTGTCGCGTTTTTCAACACTTTCCCCGTTGG
CCCTGAGCACAACCGTCTCTCACGGTCGGCTGCGTATGGTCGAGTATCGCGGAAAGGTCACAGGA
ACTCTGATCTACGACCAGCAGCCGATACTCGATCATTTCGTGATGATTGATTCGCAAACGGTACTT
GGAATTATGGATTTTAAAGAGTTCCCGCAGCCAGGCGCGTTTGTGCTGCAGCGCGATGACGACAGT
GCCGTCAGCGTTGATCGCGGCGACTGGTCCCAACTGGCGGCGCAACGGCTCGGGTGA
SEQ ID NO 62: Cupriavidus necator CnecaDUF4334 wt (WP_049800708)
MLTEMLRNRVSTTAAVLAAFDELDPLSSDSLVGCWSGFVIATGHPMDGLLSAVGWYGKMFQSVDEA
YPLIIRSPDASTLFSIDPSPLPLIGCAKLSPTDMVSRFSTLSPLALSTTVSHGRLRMVEYRGKVTGTLIYDQ
QPILDHFVMIDSQTVLGIMDFKEFPQPGAFVLQRDDDSAVSVDRGDWSQLAAQRLG*
SEQ ID NO 63: Cupriavidus necator CnecaDUF4334 E. coli optimized
ATGTTGACTGAAATGCTGCGTAACCGTGTGTCTACCACTGCCGCTGTCCTGGCCGCTTTTGACGAG
CTGGACCCGCTGTCATCCGACAGCCTGGTTGGCTGCTGGAGCGGTTTCGTTATCGCGACGGGTCAC
CCTATGGATGGTCTGCTGAGCGCGGTGGGCTGGTACGGTAAAATGTTCCAGAGCGTTGATGAAGC
ATACCCGCTGATCATCCGCTCCCCGGACGCGAGCACGCTGTTTAGCATTGATCCGTCCCCGCTGCC
GCTGATTGGTTGTGCGAAGCTGTCGCCAACCGATATGGTGAGCCGCTTCAGCACCTTAAGCCCGCT
GGCGCTGAGCACCACCGTATCTCACGGTCGTCTGCGTATGGTTGAGTATCGTGGTAAGGTTACCGG
CACGCTCATCTATGACCAACAGCCGATTTTGGATCATTTCGTCATGATTGACAGCCAAACGGTGCT
GGGCATCATGGATTTCAAAGAATTTCCGCAGCCGGGTGCGTTTGTCTTGCAGCGTGACGACGATAG
CGCAGTCAGCGTGGATCGCGGCGACTGGAGCCAACTGGCAGCCCAACGCCTGGGCTAA
SEQ ID NO 64: Cupriavidus necator CnecaDUF4334 Yeast optimized
ATGTTGACTGAAATGTTGAGAAACAGAGTTTCTACTACTGCTGCTGTTTTGGCTGCTTTCGACGAAT
TGGACCCATTGTCTTCTGACTCTTTGGTTGGTTGTTGGTCTGGTTTCGTTATCGCTACTGGTCACCCA
ATGGACGGTTTGTTGTCTGCTGTTGGTTGGTACGGTAAGATGTTCCAATCTGTTGACGAAGCTTACC
CATTGATCATCAGATCTCCAGACGCTTCTACTTTGTTCTCTATCGACCCATCTCCATTGCCATTGAT
CGGTTGTGCTAAGTTGTCTCCAACTGACATGGTTTCTAGATTCTCTACTTTGTCTCCATTGGCTTTGT
CTACTACTGTTTCTCACGGTAGATTGAGAATGGTTGAATACAGAGGTAAGGTTACTGGTACTTTGA
TCTACGACCAACAACCAATCTTGGACCACTTCGTTATGATCGACTCTCAAACTGTTTTGGGTATCAT
GGACTTCAAGGAATTCCCACAACCAGGTGCTTTCGTTTTGCAAAGAGACGACGACTCTGCTGTTTC
TGTTGACAGAGGTGACTGGTCTCAATTGGCTGCTCAAAGATTGGGTTAA
SEQ ID NO 65: Penicillium italicum PitalDUF4334-2 wt (JQGA01000120.1 65652-
66635 (+))
ATGACAATCCAATTCCCAATCATGTCATTCGACTGTTTCCAGCCAAGCCCAGCCAAGAAATTCGTC
TCTCTCACCAAACACCCTCGGGTGACTGGTGGGAAAATCAACACCGTCTTTCCTGAGCTCAAGCCT
CTTCAGCCAGACGACCTAATCGGCGAATGGGACGGATATATTCTTGTCACGGGCCACCCCTTTGAA
GAAGAACTGGACACGCTGAATTGGTTCGGAAATACATTTTATTCCACCGACGACGTGGCACCGCTG
ACTGTTGCGCGGAACGGGGTGCGGGTGCCCTTCGAGGATTGGGGGCGTGCATCTCTACGTGAAAT
CAAATATCAAGGAGTCGTCTCTGCGGCTTTGGTCTATGATAAACGACCAATGATGGTCTATTATCG
AGCCGTGAAACATAACATGGTGGCTGGGGGTATTGAGAGTAAAGAGTGGTAG
SEQ ID NO 66: Penicillium italicum PitalDUF4334-2 wt (KGO77618.1)
MTIQFPIMSFDCFQPSPAKKFVSLTKHPRVTGGKINTVFPELKPLQPDDLIGEWDGYILVTGHPFEEELDT
LNWFGNTFYSTDDVAPLTVARNGVRVPFEDWGRASLREIKYQGVVSAALVYDKRPMMVYYRAVKH
NMVAGGIESKEW*
SEQ ID NO 67: Penicillium italicum PitalDUF4334-2 E. coli optimized
ATGACCATTCAATTTCCTATCATGTCTTTTGATTGTTTTCAGCCGAGCCCAGCGAAGAAATTCGTGA
GCTTGACGAAACATCCGCGTGTTACCGGTGGCAAGATCAATACGGTTTTCCCGGAACTGAAACCGC
TGCAACCGGACGACCTGATCGGTGAGTGGGACGGTTACATTCTGGTGACGGGCCACCCGTTCGAA
GAAGAACTGGATACCTTGAACTGGTTCGGCAATACTTTCTATAGCACCGACGATGTCGCTCCGCTG
ACCGTCGCCCGCAACGGTGTGCGTGTTCCGTTTGAGGATTGGGGTCGTGCGTCCCTGCGTGAGATC
AAGTACCAGGGTGTGGTTAGCGCAGCGCTGGTCTACGACAAACGCCCGATGATGGTGTATTATCG
CGCAGTTAAGCACAACATGGTCGCGGGTGGCATTGAGAGCAAAGAGTGGTAA
SEQ ID NO 68: Ralstonia insidiosa Rins-DUF4334 wt (NZ PKPC01000002.1
18273-18773 (-))
ATGAACACGAAGCAGAAATTCGATCAACTCAAGAGCACGGAACGCCTGAATGACGAAATCCTGTT
GGAGTTCTTCGACACCCTTCCCCCCGTTTCTACGGACGAAGCGCTGGGTCGCTGGAAAGGCGGTGA
CTTCAATACGGGGCATTGGGGCAACCTCGCTCTGAAAGCAAGGAAGTGGTACGGAAAGTGGTATC
GCAGCAAGCTGGATGCGGTACCGCTTATCTGTTACGACGACCAAGGCCGCCTATATTCCAGCAAG
GCCATGAAGGGCGAAGCGTCGCTTTGGGATGTGGCGTTCCGCGGAAAGGTCTCGACCACCATGAT
CTACGACGGCGTGCCGATCTTCGATCATTTGCGCAAGGTCGACGAGAACACGCTGTTCGGCATCAT
GGATGGCAAATCGTTTGAGGGGTCCCCCGACATCATCGACCGCGGCAAGTACTACTTTTTCTACCT
CGAGAGGGTAGACAGCTTCCCGGCCGAATATCTGGAAGGCTGA
SEQ ID NO 69: Ralstonia insidiosa Rins-DUF4334 wt (WP_104654734)
MNTKQKFDQLKSTERLNDEILLEFFDTLPPVSTDEALGRWKGGDFNTGHWGNLALKARKWYGKWYR
SKLDAVPLICYDDQGRLYSSKAMKGEASLWDVAFRGKVSTTMIYDGVPIFDHLRKVDENTLFGIMDG
KSFEGSPDIIDRGKYYFFYLERVDSFPAEYLEG*
SEQ ID NO 70: Ralstonia insidiosa Rins-DUF4334 E. coli optimized
ATGAACACCAAGCAAAAGTTTGACCAGCTGAAGTCCACCGAGCGCCTGAATGATGAAATCCTGTT
GGAATTTTTCGATACCCTGCCTCCGGTGAGCACCGATGAAGCGCTGGGCCGTTGGAAGGGTGGCG
ACTTCAATACGGGTCATTGGGGTAACCTGGCCCTGAAAGCGCGTAAATGGTACGGCAAATGGTAT
CGCAGCAAACTGGACGCAGTTCCACTGATTTGCTATGACGATCAGGGCCGTCTGTACTCTAGCAAG
GCTATGAAAGGTGAGGCGAGCCTGTGGGATGTTGCGTTTCGTGGTAAAGTGAGCACGACTATGAT
CTACGACGGTGTCCCGATTTTCGACCACTTGCGTAAAGTCGATGAGAACACGCTGTTTGGTATCAT
GGATGGTAAGTCGTTCGAGGGTAGCCCGGACATTATCGACCGTGGCAAGTACTATTTCTTTTATCT
GGAGCGCGTTGACAGCTTCCCGGCAGAGTACCTGGAAGGCTAA
SEQ ID NO 71: Cryptococcus gattii EJB2 CgatDUF4334 wt (KN848661.1 262486-
263032 (-))
ATGTCCCCTCAGGAACAGTATATTGCTCTCGTCCAGGCCGGCGGCAAGTCGGACCCATCCACCATT
GAAGCTCTTTTCCAAGCGCTTCCGCCGGTCAAGCCCACTCAGCTGCTAGGCGACTGGAATCACGGC
GGATTTTTCGACACAGGCCATCCGGTTAACGAGCAACTCAAAGAGATTAAATGGATTGGAAAGTC
ATTTAAGTCCGTCGAAGATGTTGATCCTGTGATTATTGACCAGGATGGTAAGCCAACTAGCTGGAG
GAAGTGGGGGTCAGCCAGCCTGCGAGAGATGGTGTATGAAGGCACTGTATCAACGTCGATGATAT
ATGATGACCGACCAATCATCGATCACTTCCGCTACGTAGATGACGACTTTATGGCGGGGATAATGG
AAGGGAAGGCTCTGGGGGAGGCGGGGAAGTTTTATTTCTATTTGAGAAGATAG
SEQ ID NO 72: Cryptococcus gattii EJB2 CgatDUF4334 wt (KIR80015)
MSPQEQYIALVQAGGKSDPSTIEALFQALPPVKPTQLLGDWNHGGFFDTGHPVNEQLKEIKWIGKSFKS
VEDVDPVIIDQDGKPTSWRKWGSASLREMVYEGTVSTSMIYDDRPIIDHFRYVDDDFMAGIMEGKALG
EAGKFYFYLRR*
SEQ ID NO 73: Cryptococcus gattii EJB2 CgatDUF4334 E. coli optimized
ATGAGCCCACAAGAACAATACATTGCATTAGTCCAGGCCGGTGGTAAGAGCGATCCTAGCACGAT
CGAAGCGCTGTTTCAGGCATTGCCGCCGGTTAAACCGACCCAGCTGCTGGGCGATTGGAATCACG
GTGGCTTCTTTGACACGGGCCATCCGGTGAACGAACAACTGAAAGAAATCAAGTGGATTGGCAAA
TCCTTCAAATCGGTCGAAGATGTTGATCCGGTGATCATCGACCAGGACGGTAAGCCGACTAGCTGG
CGTAAGTGGGGTTCTGCGAGCCTGCGTGAGATGGTTTATGAGGGCACCGTGAGCACCAGCATGAT
TTATGACGACCGCCCGATCATTGATCACTTTCGTTACGTCGATGACGACTTCATGGCTGGTATTATG
GAAGGCAAGGCACTGGGTGAGGCCGGTAAATTCTACTTTTATCTGCGCCGTTAA
SEQ ID NO 74: Grosmannia clavigera kw1407 GclavDUF4334 wt
(XM_014316402.1)
ATGACAGCTGTACAGCGATTTAACGCACTCACCAAAGCAGAAGGGCTTCTCAAGGAGTCTGAGCT
TGCACAAATTTTCGACGAGCTCCCTCCTGTTTCTCCAGAAGCTATGACAGGCAAGTGGAATGGAGG
CAGCTTTGACAGTGGCCATCCTGTCCACAAGCTGCTTCAAACTTTTAAATGGGCAGGGAAAGAATT
CCGCTCCGTTGACGATATCGACCCGATTGTGATCTTCGACGAAAATGGGGAGCGAAAGTGGCTATC
CGAGTATGGACATGCAAGACTGCGTGAAGTTAAGTTTCGGGGAGTTGTATCTGCCGCCTTGGTATA
CGACAAAGTTGCCATTATCGACTCGTTTCGTCGGGTTTCGGACAACGTGCTGATGGGAACTATGGA
CGCCAGGGACTGGCCGGATGCTGGCATCTACTACTTTTACATCACCAAGTTTGAAGAATTGTGA
SEQ ID NO 75: Grosmannia clavigera kw1407 GclavDUF4334 wt
(XP_014171877.1)
MTAVQRFNALTKAEGLLKESELAQIFDELPPVSPEAMTGKWNGGSFDSGHPVHKLLQTFKWAGKEFR
SVDDIDPIVIFDENGERKWLSEYGHARLREVKFRGVVSAALVYDKVAIIDSFRRVSDNVLMGTMDARD
WPDAGIYYFYITKFEEL*
SEQ ID NO 76: Grosmannia clavigera kw1407 GclavDUF4334 E. coli optimized
ATGACTGCTGTTCAACGTTTTAACGCATTGACCAAAGCCGAGGGTTTGCTGAAAGAATCTGAGCTG
GCACAGATTTTCGACGAACTGCCGCCGGTTAGCCCAGAGGCCATGACCGGTAAGTGGAATGGTGG
CAGCTTTGATTCCGGCCATCCGGTGCACAAGCTGCTGCAGACGTTCAAATGGGCGGGTAAAGAATT
TCGTAGCGTTGACGACATTGACCCGATCGTGATCTTTGATGAGAATGGCGAGCGCAAGTGGCTGA
GCGAGTATGGTCACGCACGCCTGCGTGAAGTGAAGTTCCGTGGTGTCGTCAGCGCGGCTCTGGTCT
ATGACAAAGTCGCGATCATTGACAGCTTCCGCCGTGTTAGCGATAACGTGCTGATGGGTACGATGG
ATGCGCGTGATTGGCCGGATGCGGGCATTTACTACTTCTACATCACCAAGTTTGAAGAACTGTAA
SEQ ID NO 77: Oidiodendron maius Zn OmaiusDUF4334 wt (KN832882.1
673187-675938 (-))
ATGGCTTCTACTTTATATGAAGCTAGAGTTATTTTGGCACTTAAAGCTATTCAAAACAGCAACAAT
CTTAGCTTACGAGCTGCAGCAAAGCTGTATGATGTACAGCCAACAACCCTATATTACCGACAAGCT
GGCCGACCTGCACGACATGATATTCCACCTAACTCTCGCAAGCTTACGGATCTAGAAGAGGAGAC
GATTGTTCGCCCGACGGAACAGTTTATTGCCCTAGCTCAGGCCCAGGGCCGGCTTGATGCCACATT
GATTGACGCGGTGTTTAACAAGTTTGGCCCAGTCAAGCCAGAGCTGATGCTAGGCAAGTGGAGTG
GTGGGATTTTAGACACCGGCCATCCTATGGGAGATACACTGAAGGAGATACGATGGGTGGGCAAG
AATTTCACCTCCACTGAACACGTGGACCCGGTTATTATCGACAAGAACGGCCAAAGGGCCAGCTG
GGGGAAGTGGGGCCTTGCTACCCTACGTGAGGTCTTGTATCGAGATGTTGTCTCGACGGCGATGAT
CTACGATGACCGCCCGGTCTTTGACTATTTCCGTTTCGCTAATGATGATATGGTTGCTGGTATCATG
GAAGGGAAGGAGTTGGGAGGGAGACTTTTCTATTTCTACCTGAAGAGATAG
SEQ ID NO 78: Oidiodendron maius Zn OmaiusDUF4334 wt (KIM97275)
MASTLYEARVILALKAIQNSNNLSLRAAAKLYDVQPTTLYYRQAGRPARHDIPPNSRKLTDLEEETIVR
PTEQFIALAQAQGRLDATLIDAVFNKFGPVKPELMLGKWSGGILDTGHPMGDTLKEIRWVGKNFTSTE
HVDPVIIDKNGQRASWGKWGLATLREVLYRDVVSTAMIYDDRPVFDYFRFANDDMVAGIMEGKELG
GRLFYFYLKR*
SEQ ID NO 79: Oidiodendron maius Zn OmaiusDUF4334 E. coli optimized
ATGGCAAGCACTTTGTATGAAGCTCGCGTGATTCTGGCGCTGAAAGCGATTCAAAATAGCAACAA
TCTGAGCTTGCGTGCAGCCGCGAAGCTCTATGATGTCCAGCCGACCACGCTGTACTATCGTCAGGC
CGGTCGTCCAGCTCGCCACGACATCCCGCCGAACTCCCGTAAGCTGACCGATCTGGAAGAGGAAA
CGATCGTTCGCCCGACCGAGCAATTCATCGCGTTAGCACAAGCACAGGGCCGTCTGGATGCGACC
CTGATTGATGCAGTTTTCAATAAGTTTGGTCCGGTGAAGCCTGAGCTGATGCTGGGTAAGTGGAGC
GGTGGCATTCTGGACACGGGTCACCCGATGGGCGATACCCTGAAAGAAATCCGTTGGGTGGGTAA
AAATTTCACCAGCACCGAACATGTTGATCCGGTCATCATTGACAAAAACGGTCAGCGCGCTTCTTG
GGGCAAGTGGGGTCTGGCCACCTTGCGTGAAGTTCTGTACCGCGACGTCGTCAGCACGGCGATGA
TTTACGATGACCGTCCGGTGTTTGACTATTTTCGTTTCGCGAACGACGACATGGTTGCGGGTATCAT
GGAAGGCAAAGAACTGGGTGGCCGTCTGTTTTACTTCTACCTGAAACGCTAA
SEQ ID NO 80: Thermomonospora curvata TcurvaDUF4334 wt (NC_013510.1)
ATGGATGCGGAACAGCGCCTTGCCAAGATCATCGCGTCCGGCGACGAGTGCGACCGGGCCACCGT
GGAGGAACTGTACGACCGGCTGGCCCCCGTGCCGGTGGACTTCATGCTCGGCACCTGGCGGGGCG
GCATCTTCGACCGGGGCGACGCGCTGGCGGGGATGCTGCTGGGGATGAACTGGTACGGCAAGCGG
TTCATCGACCGCGACCACGTCGAGCCGCTGCTGTGCCGCTCCCCCGACGGCTCGATCTACTCCTAC
GAGAAGCTCGGGCTGGCCCGGCTGCGCGAGGTCGCCCTGCGCGGCACGGTCTCGGCGGCCATGAT
CTACGACAAGCAGCCCATCATCGACCACTTCCGGCGGGTCAACGACGACATGGTGGTCGGCGCCA
TGGACGCCAAGGGCCAGCCCGACATCCTCTACTTCCACCTCACCCGGGAACGCTGA
SEQ ID NO 81: Thermomonospora curvata TcurvaDUF4334 wt
(WP_012851400.1)
MDAEQRLAKIIASGDECDRATVEELYDRLAPVPVDFMLGTWRGGIFDRGDALAGMLLGMNWYGKRFI
DRDHVEPLLCRSPDGSIYSYEKLGLARLREVALRGTVSAAMIYDKQPIIDHFRRVNDDMVVGAMDAK
GQPDILYFHLTRER*
SEQ ID NO 82: Thermomonospora curvata TcurvaDUF4334 E. coli optimized
ATGGATGCGGAACAAAGACTGGCTAAAATTATTGCATCTGGTGATGAGTGTGATCGTGCAACCGT
GGAAGAACTGTATGACCGTTTGGCCCCTGTCCCGGTTGACTTCATGCTGGGTACGTGGCGTGGTGG
CATCTTCGATCGTGGTGATGCGCTGGCGGGTATGCTGCTGGGTATGAATTGGTATGGCAAGCGCTT
TATCGACCGCGACCACGTCGAGCCACTGCTGTGCCGTAGCCCGGATGGCTCCATCTACAGCTACGA
GAAACTGGGTCTGGCCCGTTTGCGCGAAGTGGCACTGCGTGGCACCGTTAGCGCGGCTATGATTTA
TGACAAACAGCCGATTATCGACCATTTCCGTCGCGTGAACGACGACATGGTTGTCGGCGCGATGG
ATGCGAAGGGTCAGCCGGACATCCTGTACTTTCACCTGACCCGCGAGCGTTAA
SEQ ID NO 83: Pseudomonas litoralis DlitoDUF4334 wt (NZ LT629748.1
3096922-3097413 (+))
ATGACTGCAACACTGGCCGCCCTCAGCCTGACCACCCTGCTTGCCGGGCCCAGTCTGGCCGCAGAT
ACGGAACAGCAATGGCTGGAGATGATCGCCAGCGGTGAAGCCTATTCGGCGGACACCCTGGTGCC
TCTGTTCAAACAACTCGAACCGGTGGATACCGACTTCATGGTCGGCACATGGAAGGGCGGCAAGT
TCGACGGCGGCGCCGAGCCGGACCCGATCAACTGGTACGGCAAACGTTTCACCTCGACCACCGAT
GTCGAGCCGTTATTGGTAAACGATGCCGAGGGCGAGGTGATCACCCACGACCGGCTCGGCGCCGC
ACAGATGCGCCAGGTGGTGTTCGATGGGAAGGTATCGGCCGCGTTGATCTACGACAGCCAGCCGA
TCATGGATTACCTCCGCAAGGTCAACGAGGATGTGGTCATCGGCCTGGGCGACATCAAGGGCAAG
CCTACCGATTTCTTTTTCTATCTGGTACGCGATTAA
SEQ ID NO 84: Pseudomonas litoralis DlitoDUF4334 wt (WP_090274689)
MTATLAALSLTTLLAGPSLAADTEQQWLEMIASGEAYSADTLVPLFKQLEPVDTDFMVGTWKGGKFD
GGAEPDPINWYGKRFTSTTDVEPLLVNDAEGEVITHDRLGAAQMRQVVFDGKVSAALIYDSQPIMDYL
RKVNEDVVIGLGDIKGKPTDFFFYLVRD*
SEQ ID NO 85: Pseudomonas litoralis DlitoDUF4334 E. coli optimized
ATGACTGCGACTTTGGCTGCTCTGAGCTTGACGACCCTGTTGGCTGGCCCATCTTTGGCTGCGGAC
ACCGAGCAGCAATGGCTGGAAATGATTGCAAGCGGCGAGGCGTATAGCGCGGACACCCTGGTGCC
GCTGTTCAAGCAACTGGAGCCTGTCGATACGGACTTCATGGTCGGCACGTGGAAGGGCGGCAAAT
TTGATGGTGGTGCCGAACCGGACCCGATTAACTGGTACGGTAAGCGTTTTACCAGCACGACCGATG
TGGAGCCGCTGCTGGTGAATGACGCCGAGGGTGAAGTTATCACCCACGATCGTCTGGGTGCGGCA
CAGATGCGCCAAGTTGTTTTTGATGGCAAAGTCTCCGCAGCGCTGATCTACGACAGCCAGCCGATT
ATGGACTATCTGCGCAAAGTGAACGAAGATGTTGTCATCGGTCTGGGTGACATCAAGGGTAAACC
GACCGACTTTTTCTTCTACCTGGTTCGTGATTAA
SEQ ID NO 86: Pseudomonas protegens PprotDUF4334 wt (NC_021237.1
5528027-5528524 (-))
ATGAATACGAAAGAAAAGTTTGAACAGCTTAAGAGCACGCAAGGTCTTAATGATGAAGTACTGTT
GGACTTCTTTGACTCGCTTTCTCCAGTCACAATCGATGGCGCGTTGGGCCGTTGGCAAGGTGGTGA
CTTCAAGACAGGACACTGGGGCAATGACGCACTTACCGGAATGAAGTGGTACGGAAAGTGGTACC
GGAGCAAGTTGGATGCCGTTCCCCTAGTCTGCTACGACGAACAGGGCCGACTATTTTCCAACAAGA
TCATGAAAGGTGAGGCCTCTCTCTGGGAGGTGGCGTTTCGTGGCAAGGTTTCGACTACGATGATCT
ACGATGGCGTTCCGATTTATGATCACTTGCGCAAGGTCGATGACAACACCCTTTTCGGGATCATGG
ATGGTAAGTCCTTTGAGGGGCAGCTCCCCGACATCATCGACAATGGCAAGTACTACTTCTTCTACC
TCGAAAGGGTCGATGGCTTCCCTGTCGAGTTCGTCTAG
SEQ ID NO 87: Pseudomonas protegens PprotDUF4334 wt (WP_015636872.1)
MNTKEKFEQLKSTQGLNDEVLLDFFDSLSPVTIDGALGRWQGGDFKTGHWGNDALTGMKWYGKWY
RSKLDAVPLVCYDEQGRLFSNKIMKGEASLWEVAFRGKVSTTMIYDGVPIYDHLRKVDDNTLFGIMD
GKSFEGQLPDIIDNGKYYFFYLERVDGFPVEFV*
SEQ ID NO 88: Pseudomonas protegens PprotDUF4334 E. coli optimized
ATGAACACGAAAGAAAAGTTTGAACAGTTGAAAAGCACCCAAGGTCTGAACGATGAAGTTTTGCT
GGATTTCTTCGATAGCCTGAGCCCAGTGACCATTGACGGTGCACTGGGCCGTTGGCAGGGTGGCGA
CTTCAAGACCGGTCACTGGGGCAACGACGCGCTGACTGGCATGAAATGGTACGGTAAATGGTATC
GCAGCAAACTGGATGCTGTGCCGCTGGTGTGCTACGACGAACAGGGTCGTCTGTTTTCCAATAAGA
TCATGAAAGGTGAGGCCAGCCTGTGGGAAGTCGCGTTCCGCGGTAAGGTTAGCACGACGATGATT
TATGATGGTGTGCCGATCTATGACCATCTGCGTAAAGTTGATGACAATACCCTGTTTGGCATCATG
GATGGCAAGTCTTTTGAGGGTCAACTGCCGGACATCATTGACAATGGCAAGTACTACTTCTTCTAC
CTGGAGCGTGTTGACGGTTTTCCGGTCGAGTTCGTCTAA
SEQ ID NO 89: Artificial SCH91-3944-W44A variant
MNLNEARTAFARLRAAENGLSPAELDEVWAALETVAAEEILGEAKGDDFATGHRLHEKLSASRWYGK
TFNSVEDAKPLICRDEDGNLYSDVKSGNGEASLWNIEFRGEVTATMVYDGAPIFDHFKKVDDSTLMGI
MNGKSALVLDGGQHYYFLLERA
SEQ ID NO 90: Artificial SCH91-3944-W44A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGG
CAAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 91: Artificial SCH91-3944-T51A variant
MNLNEARTAFARLRAAENGLSPAELDEVWAALETVAAEEILGEWKGDDFAAGHRLHEKLSASRWYG
KTFNSVEDAKPLICRDEDGNLYSDVKSGNGEASLWNIEFRGEVTATMVYDGAPIFDHFKKVDDSTLMG
IMNGKSALVLDGGQHYYFLLERA
SEQ ID NO 92: Artificial SCH91-3944-T51A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGGCCGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 93: Artificial SCH91-3944-H53A variant
MNLNEARTAFARLRAAENGLSPAELDEVWAALETVAAEEILGEWKGDDFATGARLHEKLSASRWYG
KTFNSVEDAKPLICRDEDGNLYSDVKSGNGEASLWNIEFRGEVTATMVYDGAPIFDHFKKVDDSTLMG
IMNGKSALVLDGGQHYYFLLERA
SEQ ID NO 94: Artificial SCH91-3944-H53A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTGCACGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 95: Artificial SCH91-3944-L59A variant
MNLNEARTAFARLRAAENGLSPAELDEVWAALETVAAEEILGEWKGDDFATGHRLHEKASASRWYG
KTFNSVEDAKPLICRDEDGNLYSDVKSGNGEASLWNIEFRGEVTATMVYDGAPIFDHFKKVDDSTLMG
IMNGKSALVLDGGQHYYFLLERA
SEQ ID NO 96: Artificial SCH91-3944-L59A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAAGCATCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 97: Artificial SCH91-3944-W64A variant
MNLNEARTAFARLRAAENGLSPAELDEVWAALETVAAEEILGEWKGDDFATGHRLHEKLSASRAYGK
TFNSVEDAKPLICRDEDGNLYSDVKSGNGEASLWNIEFRGEVTATMVYDGAPIFDHFKKVDDSTLMGI
MNGKSALVLDGGQHYYFLLERA
SEQ ID NO 98: Artificial SCH91-3944-W64A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCGCCTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 99: Artificial SCH91-3944-K67A variant
MNLNEARTAFARLRAAENGLSPAELDEVWAALETVAAEEILGEWKGDDFATGHRLHEKLSASRWYG
ATFNSVEDAKPLICRDEDGNLYSDVKSGNGEASLWNIEFRGEVTATMVYDGAPIFDHFKKVDDSTLMG
IMNGKSALVLDGGQHYYFLLERA
SEQ ID NO 100: Artificial SCH91-3944-K67A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTGCAACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 101: Artificial SCH91-3944-S71A variant
MNLNEARTAFARLRAAENGLSPAELDEVWAALETVAAEEILGEWKGDDFATGHRLHEKLSASRWYG
KTFNAVEDAKPLICRDEDGNLYSDVKSGNGEASLWNIEFRGEVTATMVYDGAPIFDHFKKVDDSTLM
GIMNGKSALVLDGGQHYYFLLERA
SEQ ID NO 102: Artificial SCH91-3944-S71A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACGCAGTTGAAGATGCAAAGCCGCTGATTTGCCGTGACGAAGATGGCAATCT
GTACTCCGATGTCAAGAGCGGTAACGGTGAGGCCAGCCTGTGGAATATCGAGTTTCGCGGCGAAG
TCACCGCGACGATGGTTTACGATGGTGCCCCGATCTTCGACCATTTCAAAAAAGTTGACGACAGCA
CCCTGATGGGCATTATGAATGGCAAAAGCGCGTTGGTGTTGGACGGTGGCCAGCATTACTATTTCC
TGCTGGAGCGTGCGTAA
SEQ ID NO 103: Artificial SCH91-3944-R106A variant
MNLNEARTAFARLRAAENGLSPAELDEVWAALETVAAEEILGEWKGDDFATGHRLHEKLSASRWYG
KTFNSVEDAKPLICRDEDGNLYSDVKSGNGEASLWNIEFAGEVTATMVYDGAPIFDHFKKVDDSTLMG
IMNGKSALVLDGGQHYYFLLERA
SEQ ID NO 104: Artificial SCH91-3944-R106A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTGCAGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 105: Artificial SCH91-3944-Y115A variant
MNLNEARTAFARLRAAENGLSPAELDEVWAALETVAAEEILGEWKGDDFATGHRLHEKLSASRWYG
KTFNSVEDAKPLICRDEDGNLYSDVKSGNGEASLWNIEFRGEVTATMVADGAPIFDHFKKVDDSTLMG
IMNGKSALVLDGGQHYYFLLERA
SEQ ID NO 106: Artificial SCH91-3944-Y115A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTGCCGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 107: Artificial SCH91-3944-D116A variant
MNLNEARTAFARLRAAENGLSPAELDEVWAALETVAAEEILGEWKGDDFATGHRLHEKLSASRWYG
KTFNSVEDAKPLICRDEDGNLYSDVKSGNGEASLWNIEFRGEVTATMVYAGAPIFDHFKKVDDSTLMG
IMNGKSALVLDGGQHYYFLLERA
SEQ ID NO 108: Artificial SCH91-3944-D116A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGCCGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 109: Artificial SCH91-3944-D122A variant
MNLNEARTAFARLRAAENGLSPAELDEVWAALETVAAEEILGEWKGDDFATGHRLHEKLSASRWYG
KTFNSVEDAKPLICRDEDGNLYSDVKSGNGEASLWNIEFRGEVTATMVYDGAPIFAHFKKVDDSTLMG
IMNGKSALVLDGGQHYYFLLERA
SEQ ID NO 110: Artificial SCH91-3944-D122A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGCACATTTCAAAAAAGTTGACGACAGCAC
CCTGATGGGCATTATGAATGGCAAAAGCGCGTTGGTGTTGGACGGTGGCCAGCATTACTATTTCCT
GCTGGAGCGTGCGTAA
SEQ ID NO 111: Artificial SCH91-3944-M136A variant
MNLNEARTAFARLRAAENGLSPAELDEVWAALETVAAEEILGEWKGDDFATGHRLHEKLSASRWYG
KTFNSVEDAKPLICRDEDGNLYSDVKSGNGEASLWNIEFRGEVTATMVYDGAPIFDHFKKVDDSTLMG
IANGKSALVLDGGQHYYFLLERA
SEQ ID NO 112: Artificial SCH91-3944-M136A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTGCAAATGGCAAAAGCGCGTTGGTGTTGGACGGTGGCCAGCATTACTATTTCCT
GCTGGAGCGTGCGTAA
SEQ ID NO 113: Artificial SCH91-3944-K139A variant
MNTNEARTAFARIRAAENGTSPARIDEVWAATRTVAAEETTGEWKGDDFATGHRIHEKISASRWYG
KTFNSVEDAKPLICRDEDGNLYSDVKSGNGEASLWNIEFRGEVTATMVYDGAPIFDHFKKVDDSTLMG
IMNGASALVLDGGQHYYFLLERA
SEQ ID NO 114: Artificial SCH91-3944-K139A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCGCAAGCGCGTTGGTGTTGGACGGTGGCCAGCATTACTATTTCCT
GCTGGAGCGTGCGTAA
SEQ ID NO 115: Artificial SCH91-3944-F152A variant
MNLNEARTAFARLRAAENGLSPAELDEVWAALETVAAEEILGEWKGDDFATGHRLHEKLSASRWYG
KTFNSVEDAKPLICRDEDGNLYSDVKSGNGEASLWNIEFRGEVTATMVYDGAPIFDHFKKVDDSTLMG
IMNGKSALVLDGGQHYYALLERA
SEQ ID NO 116: Artificial SCH91-3944-F152A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATGCACT
GCTGGAGCGTGCGTAA
SEQ ID NO 117: Artificial SCH91-3944-L154A variant
MNLNEARTAFARLRAAENGLSPAELDEVWAALETVAAEEILGEWKGDDFATGHRLHEKLSASRWYG
KTFNSVEDAKPLICRDEDGNLYSDVKSGNGEASLWNIEFRGEVTATMVYDGAPIFDHFKKVDDSTLMG
IMNGKSALVLDGGQHYYFLAERA
SEQ ID NO 118: Artificial SCH91-3944-L154A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GGCCGAGCGTGCGTAA
SEQ ID NO 119: Artificial SCH91-3944-R156A variant
MNTNEARTAFARIRAAENGTSPARIDEVWAATRTVAAEETTGEWKGDDFATGHRIHEKISASRWYG
KTFNSVEDAKPLICRDEDGNLYSDVKSGNGEASLWNIEFRGEVTATMVYDGAPIFDHFKKVDDSTLMG
IMNGKSALVLDGGQHYYFLLEAA
SEQ ID NO 120: Artificial SCH91-3944-R156A E. coli optimized
ATGAACTTAAATGAAGCGCGTACCGCATTTGCACGTCTGAGAGCTGCCGAGAACGGTCTGAGCCC
GGCTGAGCTGGATGAAGTGTGGGCAGCGCTGGAGACTGTGGCGGCTGAAGAAATCCTGGGTGAGT
GGAAGGGTGACGATTTTGCGACGGGTCACCGTCTGCACGAGAAACTGTCGGCGAGCCGCTGGTAT
GGTAAGACCTTCAACTCTGTTGAAGATGCAAAGCCGCTGATTTGCCGTGACGAAGATGGCAATCTG
TACTCCGATGTCAAGAGCGGTAACGGTGAGGCCAGCCTGTGGAATATCGAGTTTCGCGGCGAAGT
CACCGCGACGATGGTTTACGATGGTGCCCCGATCTTCGACCATTTCAAAAAAGTTGACGACAGCAC
CCTGATGGGCATTATGAATGGCAAAAGCGCGTTGGTGTTGGACGGTGGCCAGCATTACTATTTCCT
GCTGGAGGCAGCGTAA
SEQ ID NO 121: Integration cassette fragment 1
GCAGGCAGCTCCATTTCATGTAGGTGATTTATCCCTCCGGGCGGTATTTGAGACTCTCGG
SEQ ID NO 122: Integration cassette fragment 2
ACTGCTGGGTACTGTTCAGGCACGATAGGAAATGCGTCCAGCGCATACACCAGTCTTAGC
SEQ ID NO 123: Integration cassette fragment 3
AGTCGACCTTACAGCGCCTGGGACTCTACATAAACATGCAGCGAACATGCTTTCCAACGC
SEQ ID NO 124: LEU2 yeast marker primer 1
AGGTGCAGTTCGCGTGCAATTATAACGTCGTGGCAACTGTTATCAGTCGTACCGCGCCATTCGACT
ACGTCGTAAGGCC
SEQ ID NO 125: LEU2 yeast marker primer 2
TCGTGGTCAAGGCGTGCAATTCTCAACACGAGAGTGATTCTTCGGCGTTGTTGCTGACCATCGACG
GTCGAGGAGAACTT
SEQ ID NO 126: AmpR E. coli marker primer 1
TGGTCAGCAACAACGCCGAAGAATCACTCTCGTGTTGAGAATTGCACGCCTTGACCACGACACGTT
AAGGGATTTTGGTCATGAG
SEQ ID NO 127: AmpR E. coli marker primer 2
AACGCGTACCCTAAGTACGGCACCACAGTGACTATGCAGTCCGCACTTTGCCAATGCCAAAAATGT
GCGCGGAACCCCTA
SEQ ID NO 128: Yeast origin of replication primer 1
TTGGCATTGGCAAAGTGCGGACTGCATAGTCACTGTGGTGCCGTACTTAGGGTACGCGTTCCTGAA
CGAAGCATCTGTGCTTCA
SEQ ID NO 129: Yeast origin of replication primer 2
CCGAGATGCCAAAGGATAGGTGCTATGTTGATGACTACGACACAGAACTGCGGGTGACATAATGA
TAGCATTGAAGGATGAGACT
SEQ ID NO 130: E. coli replication origin primer 1
ATGTCACCCGCAGTTCTGTGTCGTAGTCATCAACATAGCACCTATCCTTTGGCATCTCGGTGAGCA
AAAGGCCAGCAAAAGG
SEQ ID NO 131: E. coli replication origin primer 2
CTCAGATGTACGGTGATCGCCACCATGTGACGGAAGCTATCCTGACAGTGTAGCAAGTGCTGAGC
GTCAGACCCCGTAGAA
SEQ ID NO 132: DNA fragment for S. cerevisiae co-transformation
ATTCCTAGTGACGGCCTTGGGAACTCGATACACGATGTTCAGTAGACCGCTCACACATGG
SEQ ID NO 133: Hyphozyma roseoniera SCH23-ADH1 wt
ATGCAATTCAGCATCGGAGATGTACTCGCCATTGTAGATAAAACAATCCTCAACCCACTCGTCGTC
AGCGCAGGACTTCTGTCTCTGCACTTTCTCACCAATGACAAATACGCAATCACTGCGAATGACGGT
CTATTCCCTTATCAAATTAGCACTCCAGACTCGCATCGAAAAGCCCTTTTTGCACTTGGCTTTGGTC
TACTTCTCAGAGCCAATCGCTACATGAGCAGAAAAGCTCTGAACAACAACACCGCCGCACAATTC
GACTGGAATCGTGAGATCATCGTTGTTACTGGTGGATCTGGTGGTATCGGTGCTCAGGCCGCGCAG
AAATTGGCAGAAAGAGGATCGAAAGTGATTGTTATTGATGTGCTACCACTTACCTTTGACAAGCCC
AAGAATTTGTACCACTATAAATGTGATCTCACAAACTACAAAGAGCTCCAAGAAGTTGCGGCTAA
GATCGAAAGAGAAGTTGGCACTCCGACTTGTGTAGTTGCGAATGCAGGAATATGTCGTGGAAAGA
ACATATTCGATGCTACAGAACGAGATGTTCAGCTTACCTTTGGAGTCAACAATCTGGGACTTCTAT
GGACAGCCAAAACCTTTCTCCCATCAATGGCCAAAGCAAATCATGGCCATTTCTTGATCATCGCCT
CTCAAACCGGCCATCTAGCGACCGCAGGAGTAGTCGACTATGCAGCGACCAAAGCAGCAGCAATC
GCCATATATGAAGGTCTACAAACAGAGATGAAGCACTTTTATAAAGCGCCTGCTGTACGCGTATCT
TGTATCTCCCCATCCGCGGTCAAGACGAAGATGTTTGCAGGCATCAAGACTGGAGGCAATTTCTTC
ATGCCAATGTTGACGCCTGATGATCTTGGAGACCTGATTGCAAAGACTTTGTGGGACGGTGTGGCA
GTCAATATTTTGAGCCCTGCGGCGGCATATATCAGCCCGCCCACGAGAGCTTTGCCAGATTGGATG
AGGGTTGGCATGCAGGATGCTGGTGCTGAGATCATGACGGAATTGACTCCTCATAAGCCGTTGGA
GTAG
SEQ ID NO 134: Hyphozyma roseonigra SCH23-ADH1 wt
MQFSIGDVLAIVDKTILNPLVVSAGLLSLHFLTNDKYAITANDGLFPYQISTPDSHRKALFALGFGLLLR
ANRYMSRKALNNNTAAQFDWNREIIVVTGGSGGIGAQAAQKLAERGSKVIVIDVLPLTFDKPKNLYHY
KCDLTNYKELQEVAAKIEREVGTPTCVVANAGICRGKNIFDATERDVQLTFGVNNLGLLWTAKTFLPS
MAKANHGHFLIIASQTGHLATAGVVDYAATKAAAIAIYEGLQTEMKHFYKAPAVRVSCISPSAVKTKM
FAGIKTGGNFFMPMLTPDDLGDLIAKTLWDGVAVNILSPAAAYISPPTRALPDWMRVGMQDAGAEIM
TELTPHKPLE
SEQ ID NO 135: Hyphozyma roseonigra SCH23-ADH1 Yeast optimized
ATGCAATTCTCTATCGGTGACGTTTTGGCTATCGTTGACAAGACTATCTTGAACCCATTGGTTGTTT
CTGCTGGTTTGTTGTCTTTGCACTTCTTGACTAACGACAAGTACGCTATCACTGCTAACGACGGTTT
GTTCCCATACCAAATCTCTACTCCAGACTCTCACAGAAAGGCTTTGTTCGCTTTGGGTTTCGGTTTG
TTGTTGAGAGCTAACAGATACATGTCTAGAAAGGCTTTGAACAACAACACTGCTGCTCAATTCGAC
TGGAACAGAGAAATCATCGTTGTTACTGGTGGTTCTGGTGGTATCGGTGCTCAAGCTGCTCAAAAG
TTGGCTGAAAGAGGTTCTAAGGTTATCGTTATCGACGTTTTGCCATTGACTTTCGACAAGCCAAAG
AACTTGTACCACTACAAGTGTGACTTGACTAACTACAAGGAATTGCAAGAAGTTGCTGCTAAGATC
GAAAGAGAAGTTGGTACTCCAACTTGTGTTGTTGCTAACGCTGGTATCTGTAGAGGTAAGAACATC
TTCGACGCTACTGAAAGAGACGTTCAATTGACTTTCGGTGTTAACAACTTGGGTTTGTTGTGGACT
GCTAAGACTTTCTTGCCATCTATGGCTAAGGCTAACCACGGTCACTTCTTGATCATCGCTTCTCAAA
CTGGTCACTTGGCTACTGCTGGTGTTGTTGACTACGCTGCTACTAAGGCTGCTGCTATCGCTATCTA
CGAAGGTTTGCAAACTGAAATGAAGCACTTCTACAAGGCTCCAGCTGTTAGAGTTTCTTGTATCTC
TCCATCTGCTGTTAAGACTAAGATGTTCGCTGGTATCAAGACTGGTGGTAACTTCTTCATGCCAAT
GTTGACTCCAGACGACTTGGGTGACTTGATCGCTAAGACTTTGTGGGACGGTGTTGCTGTTAACAT
CTTGTCTCCAGCTGCTGCTTACATCTCTCCACCAACTAGAGCTTTGCCAGACTGGATGAGAGTTGGT
ATGCAAGACGCTGGTGCTGAAATCATGACTGAATTGACTCCACACAAGCCATTGGAATAA
SEQ ID NO 136: Hyphozyma roseonigra SCH23-ADH2 wt
ATGGCGACGATACCGACCACAATGACCGCAGCGACAATCGTTGAATTCAACAAGCCCATCGTGCT
AAAGAACGACATACCAGTTCCAGACCTACCAGAGAACAAGATTCTTGTTAGGATAGCTGCAACAT
CATTATGCTCAAGCGACTTGATGGCGTACAAAGGTTACATGGATTTCATGACCAAGACGCCTTACT
GCGGAGGACACGAGCCCGTGGGAACGGTGGTGAAAGTCGGTTCTTCGGTAAAAGGCTACTCGGTT
GGAGATCGCGTTGGCATATTGATGTTCTTCGATACCTGTGGAACATGCAATGACTGCTTCTCGGGT
GAACATCGCTTTTGCAGCACAAAGAAAATCCTAGGCTTCGCGGAAAGCTGGGGAGGATTTTCAGA
ATACGCACTTGCTGATCCCATCTCGACCATCAAGCTCCCGGAAGGGTTGAGTTTCGATGTAGCAGC
GCCTTTGTTCTGCGCTGGGATCACAGCCTACAGCGCGCTGTTGAAGGTGAAGAGTCATGCCGGTCA
ACTCATCAATATCATCGGCTGTGGAGGCGTAGGACATATGGCTATATTGTATGCGAGAGCTATGGG
ATATCGAGTTCATGTTTACGATATATCCGATTCCAAGGTCGAATTTGCACTCTTTCTCGGCGCAGAT
GCAGCCTTCAACACTCTGACCTATACCGGTCCAATAGAATCAGCATCTTCTACGTTAGTCGTAAGT
GGAGCAAATGCAGCATACCAGAGCGCTCTAGGCATGACGAGTAATCATGGAGTCGTCCTGGGTAT
TGGACTACCAGCGGGAGGTGTGGTCATTGATGTGCCAGCTTGGGGTACGAAAGGCGTTACATTCGT
CCCATGCAACACAGGCTCGAAACAGGAACTAGAAGAAGCGCTAGAATTGGCTGTGAGAAAGGAT
ATCAAACCATTACTTGACATCCGCCATATCGACACAATTAATGAGGCATATCAAGATTTGGCGGAG
GGAAAGATCAATGGGAGGATTGTTTTCCACTTCGAGTGA
SEQ ID NO 137: Hyphozyma roseonigra SCH23-ADH2 wt
MATIPTTMTAATIVEFNKPIVLKNDIPVPDLPENKILVRIAATSLCSSDLMAYKGYMDFMTKTPYCGGH
EPVGTVVKVGSSVKGYSVGDRVGILMFFDTCGTCNDCFSGEHRFCSTKKILGFAESWGGFSEYALADPI
STIKLPEGLSFDVAAPLFCAGITAYSALLKVKSHAGQLINIIGCGGVGHMAILYARAMGYRVHVYDISD
SKVEFALFLGADAAFNTLTYTGPIESASSTLVVSGANAAYQSALGMTSNHGVVLGIGLPAGGVVIDVPA
WGTKGVTFVPCNTGSKQELEEALELAVRKDIKPLLDIRHIDTINEAYQDLAEGKINGRIVFHFE
SEQ ID NO 138: Hyphozyma roseonigra SCH23-ADH2 Yeast optimized
ATGGCTACTATCCCAACTACTATGACTGCTGCTACTATCGTTGAATTCAACAAGCCAATCGTTTTGA
AGAACGACATCCCAGTTCCAGACTTGCCAGAAAACAAGATCTTGGTTAGAATCGCTGCTACTTCTT
TGTGTTCTTCTGACTTGATGGCTTACAAGGGTTACATGGACTTCATGACTAAGACTCCATACTGTGG
TGGTCACGAACCAGTTGGTACTGTTGTTAAGGTTGGTTCTTCTGTTAAGGGTTACTCTGTTGGTGAC
AGAGTTGGTATCTTGATGTTCTTCGACACTTGTGGTACTTGTAACGACTGTTTCTCTGGTGAACACA
GATTCTGTTCTACTAAGAAGATCTTGGGTTTCGCTGAATCTTGGGGTGGTTTCTCTGAATACGCTTT
GGCTGACCCAATCTCTACTATCAAGTTGCCAGAAGGTTTGTCTTTCGACGTTGCTGCTCCATTGTTC
TGTGCTGGTATCACTGCTTACTCTGCTTTGTTGAAGGTTAAGTCTCACGCTGGTCAATTGATCAACA
TCATCGGTTGTGGTGGTGTTGGTCACATGGCTATCTTGTACGCTAGAGCTATGGGTTACAGAGTTC
ACGTTTACGACATCTCTGACTCTAAGGTTGAATTCGCTTTGTTCTTGGGTGCTGACGCTGCTTTCAA
CACTTTGACTTACACTGGTCCAATCGAATCTGCTTCTTCTACTTTGGTTGTTTCTGGTGCTAACGCT
GCTTACCAATCTGCTTTGGGTATGACTTCTAACCACGGTGTTGTTTTGGGTATCGGTTTGCCAGCTG
GTGGTGTTGTTATCGACGTTCCAGCTTGGGGTACTAAGGGTGTTACTTTCGTTCCATGTAACACTGG
TTCTAAGCAAGAATTGGAAGAAGCTTTGGAATTGGCTGTTAGAAAGGACATCAAGCCATTGTTGG
ACATCAGACACATCGACACTATCAACGAAGCTTACCAAGACTTGGCTGAAGGTAAGATCAACGGT
AGAATCGTTTTCCACTTCGAATAA
SEQ ID NO 139: Filobasidium magnum SCH24-ADH1 wt
ATGCCAACCCCTATCTTTGGCGCCCGAGAGGGTTTCACTATCGACTCCGTACTGAGCATCCTGGAT
GCGACCGTACTTAACCCCTGGTTTACCGGCGTGTGCCTAATAGCCGTCTGCGCCCGAGATCGCACC
ATTACGTACCCGGACTGGCCGGCGGCTCTGGACCAGGTGCTCCCCTTCTTGTCGCAGATGTGGAGG
GAAACTGTCAGACCGACCTTTGGCGACCGCAACGTCCTTCATCTGTTGACCACTGTGTGTGTCGGC
CTTGCCATCCGAACCAACAGACGGATGAGTCGGGGAGCGAGGAACAATTGGGTGTGGGATACTAG
TTATGACTGGAAGAAGGAGATCGTAGTGGTTACGGGAGGAGCTGCCGGGTTTGGTGCAGACATCG
TACAACAGCTAGACACGCGTGGAATCCAGGTCGTCGTCTTGGATGTGGGATCCCTCACCTATAGGC
CTTCGAGCAGAGTTCATTATTACAAGTGCGACGTGTCGAACCCACAAGACGTCGCCAGCGTGGCTA
AAGCTATCGTATCCAACGTCGGGCACCCGACCATATTGGTCAACAACGCTGGCGTATTCAGGGGTG
CGACTATTCTCTCCACGACACCGCGCGACCTCGACATGACCTACGACATCAACGTCAAAGCGCACT
ATCATCTCACGAAGGCGTTCCTCCCGAACATGATCTCCAAGAACCATGGACATATTGTGACTGTGT
CAAGCGCGACCGCATACGCTCAAGCTTGTTCTGGCGTGTCATACTGTTCCTCAAAGGCCGCCATCT
TGTCATTTCACGAAGGACTGAGCGAAGAGATTTTGTGGATCTATAAGGCGCCCAAAGTCCGGACCT
CGGTCATCTGCCCCGGACACGTCAATACGGCCATGTTTACAGGCATTGGAGCCGCCGCTCCCTCGT
TCATGGCACCTGCACTTCATCCCTCGACAGTCGCCGAGACAATCGTCGATGTATTGCTCTCATGCG
AGTCTCAACACGTCCTGATGCCCGCCGCCATGCACATGTCAGTCGCCGGACGAGCGCTGCCCACCT
GGTTCTTCCGGGGGTTGTTGGCATCGGGCAAGGATACCATGGGTAGCGTTGTCCGCCGATGA
SEQ ID NO 140: Filobasidium magnum SCH24-ADH1 wt
MPTPIFGAREGFTIDSVLSILDATVLNPWFTGVCLIAVCARDRTITYPDWPAALDQVLPFLSQMWRETV
RPTFGDRNVLHLLTTVCVGLAIRTNRRMSRGARNNWVWDTSYDWKKEIVVVTGGAAGFGADIVQQL
DTRGIQVVVLDVGSLTYRPSSRVHYYKCDVSNPQDVASVAKAIVSNVGHPTILVNNAGVFRGATILSTT
PRDLDMTYDINVKAHYHLTKAFLPNMISKNHGHIVTVSSATAYAQACSGVSYCSSKAAILSFHEGLSEE
ILWIYKAPKVRTSVICPGHVNTAMFTGIGAAAPSFMAPALHPSTVAETIVDVLLSCESQHVLMPAAMH
MSVAGRALPTWFFRGLLASGKDTMGSVVRR*
SEQ ID NO 141: Filobasidium magnum SCH24-ADH1 Yeast optimized
ATGCCAACTCCAATCTTCGGTGCTAGAGAAGGTTTCACTATCGACTCTGTTTTGTCTATCTTGGACG
CTACTGTTTTGAACCCATGGTTCACTGGTGTTTGTTTGATCGCTGTTTGTGCTAGAGACAGAACTAT
CACTTACCCAGACTGGCCAGCTGCTTTGGACCAAGTTTTGCCATTCTTGTCTCAAATGTGGAGAGA
AACTGTTAGACCAACTTTCGGTGACAGAAACGTTTTGCACTTGTTGACTACTGTTTGTGTTGGTTTG
GCTATCAGAACTAACAGAAGAATGTCTAGAGGTGCTAGAAACAACTGGGTTTGGGACACTTCTTA
CGACTGGAAGAAGGAAATCGTTGTTGTTACTGGTGGTGCTGCTGGTTTCGGTGCTGACATCGTTCA
ACAATTGGACACTAGAGGTATCCAAGTTGTTGTTTTGGACGTTGGTTCTTTGACTTACAGACCATCT
TCTAGAGTTCACTACTACAAGTGTGACGTTTCTAACCCACAAGACGTTGCTTCTGTTGCTAAGGCT
ATCGTTTCTAACGTTGGTCACCCAACTATCTTGGTTAACAACGCTGGTGTTTTCAGAGGTGCTACTA
TCTTGTCTACTACTCCAAGAGACTTGGACATGACTTACGACATCAACGTTAAGGCTCACTACCACT
TGACTAAGGCTTTCTTGCCAAACATGATCTCTAAGAACCACGGTCACATCGTTACTGTTTCTTCTGC
TACTGCTTACGCTCAAGCTTGTTCTGGTGTTTCTTACTGTTCTTCTAAGGCTGCTATCTTGTCTTTCC
ACGAAGGTTTGTCTGAAGAAATCTTGTGGATCTACAAGGCTCCAAAGGTTAGAACTTCTGTTATCT
GTCCAGGTCACGTTAACACTGCTATGTTCACTGGTATCGGTGCTGCTGCTCCATCTTTCATGGCTCC
AGCTTTGCACCCATCTACTGTTGCTGAAACTATCGTTGACGTTTTGTTGTCTTGTGAATCTCAACAC
GTTTTGATGCCAGCTGCTATGCACATGTCTGTTGCTGGTAGAGCTTTGCCAACTTGGTTCTTCAGAG
GTTTGTTGGCTTCTGGTAAGGACACTATGGGTTCTGTTGTTAGAAGATAA
SEQ ID NO 142: Filobasidium magnum SCH24-ADH2 wt
ATGGAGCCACCCCAGACTATGAAGGCCGCCTTGGTCACCGCATACAACGAGCCCCTGATTGTGAA
AGACGTTGCTACACCCGAGCCGGGCCCTGGACAGATTCTCGTTCGGGTCAAAGCTAGTTCGCTTTG
CATGTCAGATATCGGAGGCTATGTCGGAGCGATGGGGGAATTTATCACGCTCCCCTATTGTCCAGG
TCATGAACCCGCCGGAGAGATCGTCGCCCTTGGCGACAACGTGTCCGGCTTCTCCGTTGGGGATCG
GGTTACTTATATGGCCGCTCTAGATCCTTGTATGGGCTGCCGAGACTGTCTCCGAGGTGCCATTCG
ATTCTGCTCCAAACGCTCGAATCTCGGCTTCAGCCACCAGTACGGCGGGTTCTCCGAGTACTCTCT
CGCCAGCCCATACTCGATGGCCAAGGTGCCGGACGAACTGTCCCTCGAGGAAGCTGCGAGCATGT
CCTGTGCCGGGGTGACCGCTTTCGGTGCTCTCAAGCTATTGAGCAAGTATCAGGCTCCGGGAGGCA
TCATCAATGTCTTGGGCTGCGGCGGCGTTGGTCATCTGGTCATCAAGTTTGCCGTCGCGCTCGGCT
ACACCGTGCACGCTTTCGACATTAACGATGGCAAACTCAAACTGGCCGAGGAGTGCGGGGCATCT
AAAGCCTTCCTTTCAAAGGGAGATCCCACCCAGGCGATGATGGCAGAGAGTACGATAGTCATCTC
GGGTGTCAACGCGGCCTATGATTTCGCTATTAAAGCTACTCTGGCCGGTGGACGTATCATTGCGAT
TGGGCACCCACATTCGGCGACTCCGATGCCTCTCGGCTCGATGATCATCAACGACATCTCGTTGAT
CGTGAGCAATCAAGGTACAAGGGTGGATCTACAAGAAGCCTTGGATTTCGCCGCTCGATCCGGTG
TCAGACCGAACATTACAATCAACGAAGGTCTGGACGGCATCAATCAGGGCTATAAATCGGTCATG
ACAGGCGCTGTAGAAGGCAGATTGGTCTACAAATTCTAG
SEQ ID NO 143: Filobasidium magnum SCH24-ADH2 wt
MEPPQTMKAALVTAYNEPLIVKDVATPEPGPGQILVRVKASSLCMSDIGGYVGAMGEFITLPYCPGHEP
AGEIVALGDNVSGFSVGDRVTYMAALDPCMGCRDCLRGAIRFCSKRSNLGFSHQYGGFSEYSLASPYS
MAKVPDELSLEEAASMSCAGVTAFGALKLLSKYQAPGGIINVLGCGGVGHLVIKFAVALGYTVHAFDI
NDGKLKLAEECGASKAFLSKGDPTQAMMAESTIVISGVNAAYDFAIKATLAGGRIIAIGHPHSATPMPL
GSMIINDISLIVSNQGTRVDLQEALDFAARSGVRPNITINEGLDGINQGYKSVMTGAVEGRLVYKF
SEQ ID NO 144: Filobasidium magnum SCH24-ADH2 Yeast optimized
ATGGAACCACCACAAACTATGAAGGCTGCTTTGGTTACTGCTTACAACGAACCATTGATCGTTAAG
GACGTTGCTACTCCAGAACCAGGTCCAGGTCAAATCTTGGTTAGAGTTAAGGCTTCTTCTTTGTGT
ATGTCTGACATCGGTGGTTACGTTGGTGCTATGGGTGAATTCATCACTTTGCCATACTGTCCAGGTC
ACGAACCAGCTGGTGAAATCGTTGCTTTGGGTGACAACGTTTCTGGTTTCTCTGTTGGTGACAGAG
TTACTTACATGGCTGCTTTGGACCCATGTATGGGTTGTAGAGACTGTTTGAGAGGTGCTATCAGAT
TCTGTTCTAAGAGATCTAACTTGGGTTTCTCTCACCAATACGGTGGTTTCTCTGAATACTCTTTGGC
TTCTCCATACTCTATGGCTAAGGTTCCAGACGAATTGTCTTTGGAAGAAGCTGCTTCTATGTCTTGT
GCTGGTGTTACTGCTTTCGGTGCTTTGAAGTTGTTGTCTAAGTACCAAGCTCCAGGTGGTATCATCA
ACGTTTTGGGTTGTGGTGGTGTTGGTCACTTGGTTATCAAGTTCGCTGTTGCTTTGGGTTACACTGT
TCACGCTTTCGACATCAACGACGGTAAGTTGAAGTTGGCTGAAGAATGTGGTGCTTCTAAGGCTTT
CTTGTCTAAGGGTGACCCAACTCAAGCTATGATGGCTGAATCTACTATCGTTATCTCTGGTGTTAAC
GCTGCTTACGACTTCGCTATCAAGGCTACTTTGGCTGGTGGTAGAATCATCGCTATCGGTCACCCA
CACTCTGCTACTCCAATGCCATTGGGTTCTATGATCATCAACGACATCTCTTTGATCGTTTCTAACC
AAGGTACTAGAGTTGACTTGCAAGAAGCTTTGGACTTCGCTGCTAGATCTGGTGTTAGACCAAACA
TCACTATCAACGAAGGTTTGGACGGTATCAACCAAGGTTACAAGTCTGTTATGACTGGTGCTGTTG
AAGGTAGATTGGTTTACAAGTTCTAA
SEQ ID NO 145: Rhodococcus sp. RrhSecADH wt (NZ AZHI01000124.1 6627-
7664 (+))
ATGAAAGCCGTCCAGTACACCGAGATCGGCTCCGAGCCGGTCGTTGTCGACATCCCCACCCCGAC
GCCCGGGCCGGGTGAGATCCTGCTGAAGGTCACCGCGGCCGGGCTGTGCCACTCGGACATCTTCGT
GATGGACATGCCGGCGGCGCAGTACGCCTACGGCCTGCCGCTCACCCTCGGCCACGAGGGTGTCG
GCACCGTCGCCGAACTCGGCGAGGGCGTCACGGGATTCGGGGTGGGGGACGCCGTCGCCGTGTAC
GGGCCGTGGGGCTGCGGTGCGTGCCACGCCTGCGCGCGCGGCCGGGAGAACTACTGCACCCGCGC
CGCCGACCTGGGCATCACGCCACCCGGTCTCGGCTCGCCCGGATCGATGGCCGAGTACATGATCGT
CGATTCGGCGCGCCACCTCGTCCCGATCGGAGACCTCGACCCGGTCGCCGCGGCGCCGCTCACCGA
CGCCGGTCTGACGCCGTACCACGCGATCTCCCGGGTCCTGCCGCTGCTGGGGCCGGGCTCGACGGC
CGTCGTCATCGGTGTCGGCGGGCTCGGCCACGTCGGCATCCAGATCCTGCGCGCCGTCAGCGCGGC
CCGTGTGATCGCCGTCGACCTCGACGACGACCGTCTCGCCCTCGCCCGCGAGGTCGGCGCCGACGC
GGCGGTGAAGTCGGGCGCCGGTGCGGCGGACGCGATCCGGGAACTGACCGGCGGCCAGGGCGCG
ACGGCGGTGTTCGACTTCGTCGGCGCCCAGTCGACGATCGACACGGCGCAGCAGGTGGTCGCGGT
CGACGGGCACATCTCGGTCGTGGGCATCCACGCCGGCGCACACGCCAAGGTCGGGTTCTTCATGAT
CCCGTTCGGCGCCTCCGTCGTGACCCCGTACTGGGGCACCCGGTCGGAACTGATGGAGGTCGTCGC
GCTGGCCCGCGCCGGCCGGCTGGACATCCACACCGAGACGTTCACCCTCGACGAGGGGCCGGCGG
CGTACCGGCGGCTGCGCGAGGGCAGCATCCGCGGCCGCGGCGTGGTGGTTCCCTGA
SEQ ID NO 146: Rhodococcus sp. RrhSecADH wt (WP_043801412.1)
MKAVQYTEIGSEPVVVDIPTPTPGPGEILLKVTAAGLCHSDIFVMDMPAAQYAYGLPLTLGHEGVGTV
AELGEGVTGFGVGDAVAVYGPWGCGACHACARGRENYCTRAADLGITPPGLGSPGSMAEYMIVDSA
RHLVPIGDLDPVAAAPLTDAGLTPYHAISRVLPLLGPGSTAVVIGVGGLGHVGIQILRAVSAARVIAVDL
DDDRLALAREVGADAAVKSGAGAADAIRELTGGQGATAVFDFVGAQSTIDTAQQVVAVDGHISVVGI
HAGAHAKVGFFMIPFGASVVTPYWGTRSELMEVVALARAGRLDIHTETFTLDEGPAAYRRLREGSIRG
RGVVVP*
SEQ ID NO 147: Rhodococcus sp. RrhSecADH E. coli optimized
ATGAAAGCAGTGCAATATACGGAAATTGGCTCGGAACCTGTTGTGGTGGACATCCCGACCCCGAC
CCCGGGTCCTGGTGAAATCCTGCTGAAAGTTACCGCTGCCGGCCTGTGCCACAGCGACATCTTCGT
GATGGACATGCCGGCTGCCCAGTACGCTTACGGTCTGCCACTGACGCTGGGTCACGAAGGCGTGG
GTACGGTCGCCGAACTGGGCGAGGGTGTCACCGGTTTCGGTGTCGGTGATGCCGTGGCAGTGTAC
GGTCCGTGGGGTTGCGGTGCGTGCCACGCGTGTGCGCGCGGCCGCGAGAATTACTGTACGCGTGC
AGCGGACCTGGGTATTACCCCGCCGGGCCTGGGCAGCCCGGGTAGCATGGCAGAGTACATGATCG
TTGATAGCGCACGTCATCTGGTGCCGATTGGCGATTTGGACCCGGTCGCGGCAGCCCCGCTGACTG
ACGCGGGCTTGACCCCGTATCATGCAATCTCCCGTGTACTGCCACTGCTCGGTCCGGGCAGCACCG
CTGTGGTTATCGGCGTCGGTGGCCTGGGTCATGTTGGCATCCAGATTCTGCGTGCAGTCAGCGCGG
CACGCGTGATCGCGGTTGATCTGGACGACGATCGCCTGGCCCTGGCGCGTGAGGTCGGTGCGGAT
GCTGCGGTTAAGTCTGGTGCCGGTGCAGCGGATGCGATTCGTGAGCTGACGGGTGGCCAGGGTGC
GACCGCCGTGTTTGATTTCGTTGGCGCGCAGAGCACCATTGATACGGCGCAACAAGTCGTTGCGGT
CGATGGTCACATTTCCGTTGTGGGTATCCACGCGGGTGCACATGCCAAGGTCGGCTTTTTCATGAT
CCCGTTTGGTGCTAGCGTTGTTACCCCGTATTGGGGCACGCGCAGCGAGCTGATGGAAGTCGTGGC
TTTGGCGCGTGCGGGTCGTCTGGACATTCACACCGAGACTTTCACCTTGGACGAAGGCCCGGCAGC
GTATCGTCGTCTGCGCGAGGGTAGCATTCGTGGCCGTGGTGTTGTTGTCCCGTAA
SEQ ID NO 148: Rhodococcus rhodochrous SCH80-00043 wt
ATGAAGACCAAAGCTGCTGTACTGCTCGAGCCCGGAAAGCCTTTCGAGATCATGGAACTCGACCT
CGACGGCCCGGGTGTGGGTGAGGTACTGATCAAGTACACCGCTGCCGGACTGTGCCATTCGGATCT
GCACCTGACCGACGGTGATCTCCCGCCGCGTTACCCGATCGTCGGCGGACACGAAGGCTCGGGCA
TCATCGAAGAGGTCGGCCCAGGCGTCACGAAGGTCAAGCCGGGCGACCATGTCGTGTGTAGCTTC
ATCCCCAACTGCGGCACCTGTCGCTACTGCTCGACCGGTCGCCAGAACCTCTGCGACATGGGTGCC
ACCATCCTCGAAGGCTCGATGCCCGACGGTTCCTTCCGTTTCCACGGCAACGGAATGGATTTCGGC
GGAATGTGCATGTTGGGAACGTTCTCCGAGCGCGCCACCATTTCTCAGCACTCGGTAGTCAAGATC
GACGACTGGCTTCCCTTGGAGACAGCGGTGGTCGTCGGCTGCGGCGTGCCTTCGGGTTGGGGAAC
GGCAGTAAATGCCGGTAACCTTCGCGCCGGTGACACCGCTGTGATCTACGGCATCGGTGGTCTCGG
CATCAACGCCGTCCAGGGCGCCGTTTCGGCCGGCTGCAAGTACGTCGTTGTGGTCGATCCGGTTGC
TCTCAAGCGTGAGACCGCACTGAAGTTCGGTGCAACCCATGCCTTTGCAGACGCCGAGAGCGCTG
CTGCCAAGGTCAACGAGCTGACGTGGGGACAGGGTGCCGACGCTGCGCTCATCCTTGTCGGCACC
GTCGACGAGGACGTGGTCAGTGCAGCGACGGCAGTGATCGGCAAGGGTGGCACCGTGGTGATCAC
GGGACTCGCCGACCCCGCCAAGCTGACCGTTCACGTGTCGGGTACCGACCTGACGCTGAATCAGA
AGACGATCAAGGGCACGTTGTTCGGGTCCATGAATCCGCAGTACGACATCGTGCGACTGCTGCGTC
TCTACGATGCCGGTCAGCTCAAGCTCGACGAACTGATCACCAACACCTACAGCCTCGAAGACGTC
AACAAGGGCTACCAGGATCTACGTGACGGCAAGAACATCCGTGGCGTGATCATTCACGACAAGTA
A
SEQ ID NO 149: Rhodococcus rhodochrous SCH80-00043 wt
MKTKAAVLLEPGKPFEIMELDLDGPGVGEVLIKYTAAGLCHSDLHLTDGDLPPRYPIVGGHEGSGIIEE
VGPGVTKVKPGDHVVCSFIPNCGTCRYCSTGRQNLCDMGATILEGSMPDGSFRFHGNGMDFGGMCML
GTFSERATISQHSVVKIDDWLPLETAVVVGCGVPSGWGTAVNAGNLRAGDTAVIYGIGGLGINAVQGA
VSAGCKYVVVVDPVALKRETALKFGATHAFADAESAAAKVNELTWGQGADAALILVGTVDEDVVSA
ATAVIGKGGTVVITGLADPAKLTVHVSGTDLTLNQKTIKGTLFGSMNPQYDIVRLLRLYDAGQLKLDE
LITNTYSLEDVNKGYQDLRDGKNIRGVIIHDK*
SEQ ID NO 150: Rhodococcus rhodochrous SCH80-00043 E. coli optimized
ATGAAAACGAAAGCCGCAGTGTTGTTGGAGCCGGGCAAACCATTTGAGATCATGGAACTGGATCT
GGACGGTCCGGGTGTCGGTGAAGTGCTGATCAAGTACACCGCAGCGGGCTTGTGCCACTCTGATCT
GCACCTGACCGACGGCGACTTGCCGCCACGTTACCCGATTGTGGGTGGCCATGAGGGTAGCGGTA
TCATTGAAGAGGTTGGTCCGGGCGTTACCAAGGTCAAACCGGGTGATCACGTCGTGTGCTCTTTCA
TCCCGAATTGTGGTACGTGCCGCTATTGTAGCACGGGTCGTCAGAACCTGTGCGACATGGGTGCCA
CCATTTTAGAGGGCTCCATGCCTGATGGCTCCTTCCGTTTTCACGGCAACGGTATGGACTTTGGTGG
CATGTGCATGCTGGGTACGTTCAGCGAACGCGCGACCATCAGCCAACATAGCGTCGTTAAGATCG
ATGACTGGCTCCCGCTGGAAACCGCAGTTGTTGTTGGTTGTGGTGTTCCGAGCGGTTGGGGTACTG
CGGTCAATGCCGGTAATCTGCGTGCTGGTGACACCGCGGTCATTTATGGTATTGGCGGCCTGGGTA
TCAACGCTGTGCAGGGCGCAGTTAGCGCGGGCTGCAAATACGTCGTTGTGGTTGACCCGGTTGCGC
TGAAACGTGAGACTGCGCTGAAATTTGGCGCAACCCACGCGTTCGCAGACGCGGAGAGCGCAGCT
GCGAAAGTGAACGAACTGACCTGGGGTCAGGGTGCGGATGCGGCACTGATCTTGGTCGGCACCGT
GGACGAAGATGTCGTGAGCGCGGCGACTGCTGTTATCGGTAAGGGTGGCACCGTTGTGATCACCG
GTCTGGCCGATCCGGCAAAGCTGACCGTTCATGTCAGCGGTACGGACCTGACCCTGAATCAGAAA
ACCATTAAGGGCACGCTGTTCGGTTCGATGAACCCGCAGTACGACATTGTGCGCCTGCTGCGTCTG
TACGATGCGGGTCAACTGAAACTGGACGAACTTATTACGAATACGTATAGCCTGGAAGATGTGAA
CAAAGGCTACCAAGATCTGCGTGATGGTAAGAATATTCGTGGTGTCATTATCCACGACAAGTGA
SEQ ID NO 151: Rhodococcus rhodochrous SCH80-04254 wt
ATGAAGGCAGCCCAGCTCATGGGGCCCGGGCTCCTGGAAATCAACGACGTGCCGGTCCCGGAGAT
CGGCCCGTCGGAACTACTGATTCGGGTGGGCGCAGCGGGAATCTGCCACTCCGATCTCCATCTCCT
GCACTTTCCGTACAAGATGCGCGAAGAACCGCTGACAATCGGCCACGAAATTGCCGGAACGATCG
AAGCCGTCGGGAGTGGCGTCGACGGCCGTTCCGTCGGAGAGCGTGGCGTCGTCTACCTCTGTTGGT
CATGTGGACAGTGCCGAGAATGCATGAGCGGCAACGAGAATATGTGCCTCGCCGCTGGACGCACC
GCGATGCCGCCCTGCCCCGGACTCGGCCCTGAGGGCGGGATGGCCGAGTACGTCAAGATCCCGGC
TCGCTCATTCGTACCCATCGGAGACCTCGACTTCCTGCAGGCCGCACCTCTCGCCGATGCGGCACT
GACGAGCTACCACGCCATTCGCGGTGCCCGCGAACATCTCCAGCCCGGTGCCACCGCCGTCGTGAT
CGGCGTCGGCGGACTCGGTCACGTTGCAGTACAGATACTTCGCGCGATCAGTGCCGTGCGCATCAT
CGCCGTCGATGTCGGACAGGATCAACTCGATCTCGCCAAACGTTGCGGCGCCGACATCACGCTCG
AATCGGGACCGGACACCGCGCAGCACATCCTCGACCTCACATCGGCCAGAGGCGCAGAAGTCATC
TTCGACTTCGTCGGTATCGACGCAACTGCACAGATGTCTGTTCAAGCGGTTGCGCCGAACGGCGCG
TATCGCATGGTAGGTCTCGGAGGCGGAAACCCCGGAATCACTGCCGAAGCTGCCGGCGGACCAGG
CTGGCCATGGGGCGCATCGATCCGGAAGTCCTACGGCGGCACCAGAAACGACCTCGTCGATTCCA
TCGCCCTGGCACAGGCCGGTCTGGTAACGGTAGAAGTAGCCCGCTTCGACCTCGCTGATGCCCGCG
ACGCACTCGACCGTCTCGAACACGGCAAGGTCACCGGACGCGCAGTGCTCGTACCCTGA
SEQ ID NO 152: Rhodococcus rhodochrous SCH80-04254 wt
MKAAQLMGPGLLEINDVPVPEIGPSELLIRVGAAGICHSDLHLLHFPYKMREEPLTIGHEIAGTIEAVGS
GVDGRSVGERGVVYLCWSCGQCRECMSGNENMCLAAGRTAMPPCPGLGPEGGMAEYVKIPARSFVPI
GDLDFLQAAPLADAALTSYHAIRGAREHLQPGATAVVIGVGGLGHVAVQILRAISAVRIIAVDVGQDQ
LDLAKRCGADITLESGPDTAQHILDLTSARGAEVIFDFVGIDATAQMSVQAVAPNGAYRMVGLGGGNP
GITAEAAGGPGWPWGASIRKSYGGTRNDLVDSIALAQAGLVTVEVARFDLADARDALDRLEHGKVTG
RAVLVP*
SEQ ID NO 153: Rhodococcus rhodochrous SCH80-04254 E. coli optimized
ATGAAAGCTGCACAACTGATGGGTCCGGGTCTGTTGGAAATTAATGATGTTCCAGTCCCAGAAATT
GGTCCGAGCGAGCTGCTGATCCGTGTTGGCGCTGCCGGCATTTGCCACAGCGATCTGCATCTGCTG
CACTTCCCGTACAAGATGCGTGAGGAACCGTTAACCATTGGTCACGAAATCGCGGGCACGATCGA
AGCCGTTGGTAGCGGTGTGGATGGCCGCAGCGTTGGTGAGCGTGGCGTGGTTTACCTGTGCTGGTC
CTGTGGTCAGTGCCGCGAGTGCATGTCCGGCAATGAAAACATGTGTCTGGCGGCTGGTCGTACCGC
AATGCCGCCATGTCCGGGTTTGGGTCCTGAGGGTGGCATGGCCGAATATGTCAAGATCCCGGCGC
GTAGCTTCGTGCCGATTGGCGATCTGGACTTTCTGCAGGCAGCGCCTTTGGCGGACGCAGCACTGA
CCAGCTACCACGCGATCCGTGGTGCCCGCGAACACTTGCAGCCGGGTGCAACCGCAGTGGTCATT
GGTGTCGGCGGCTTGGGTCATGTGGCAGTGCAAATCCTGCGCGCGATTTCTGCGGTCCGTATCATT
GCGGTTGATGTGGGCCAGGACCAACTGGACCTGGCGAAGCGTTGTGGCGCGGACATCACCCTGGA
GAGCGGTCCTGACACCGCGCAACATATCCTGGACCTGACCTCCGCTCGTGGTGCCGAAGTGATTTT
TGACTTCGTCGGTATCGATGCGACGGCACAGATGAGCGTCCAAGCGGTAGCCCCGAATGGCGCAT
ACCGTATGGTTGGTCTGGGTGGTGGCAACCCGGGCATTACTGCAGAGGCAGCGGGTGGTCCTGGTT
GGCCGTGGGGTGCTTCGATCCGCAAAAGCTATGGCGGCACGCGTAACGACCTGGTTGATTCTATTG
CGTTGGCCCAGGCTGGTCTTGTTACCGTTGAAGTGGCGCGCTTTGACCTGGCAGACGCCCGTGATG
CGCTGGACCGTCTGGAGCATGGTAAAGTGACGGGTCGCGCTGTGCTGGTGCCGTAA
SEQ ID NO 154: Rhodococcus rhodochrous SCH80-06135 wt
ATGAAGGCAATCCAGTACACGAGAATCGGCGCAGAACCCGAACTCACGGAGATTCCCAAACCCGA
GCCCGGTCCAGGTGAAGTGCTCCTGGAAGTCACCGCTGCCGGCGTCTGCCACTCGGACGACTTCAT
CATGAGCCTGCCCGAAGAGCAGTACACCTACGGCCTTCCTCTCACGCTCGGCCACGAAGGCGCCG
GCCGGGTCGCCGCCGTCGGCGAGGGCGTCGAAGGACTCGACATCGGAACCAATGTCGTCGTCTAC
GGACCCTGGGGCTGTGGCAGCTGTTGGCACTGCTCGCAAGGACTCGAAAACTACTGTTCTCGGGCA
AAAGAACTCGGCATCAATCCTCCTGGTCTCGGTGCACCCGGCGCGTTGGCCGAATTCATGATCGTC
GATTCACCTCGCCACCTCGTCCCGATCGGCGACCTCGATCCGGTCAAGACGGTGCCGCTGACCGAC
GCCGGTCTGACTCCGTATCACGCGATCAAGCGTTCACTGCCGAAACTTCGCGGTGGCGCGTACGCC
GTCGTCATCGGTACCGGCGGTCTCGGCCATGTCGCCATCCAACTCCTCCGCCACCTCTCGGCAGCA
ACCGTCATCGCACTCGACGTGAGCGCGGACAAGCTCGAACTGGCAACCAAGGTAGGCGCTCACGA
AGTGGTCCTGTCCGACAAGGACGCGGCCGAGAACGTCCGCAGGATCACCGGAAGTCAGGGCGCCG
CACTGGTTCTCGACTTCGTCGGCTATCAGCCCACCATCGACACCGCGATGGCTGTCGCCGGCGTCG
GATCGGACGTCACGATCGTCGGGATCGGCGACGGGCAGGCCCATGCCAAAGTCGGGTTCTTCCAA
AGTCCTTACGAGGCTTCTGTGACAGTTCCGTACTGGGGTGCCCGCAACGAGCTGATCGAATTGATC
GACCTGGCGCACGCCGGCATCTTCGACATCGCGGTGGAGACCTTCAGTCTCGACAACGGCGCCGA
AGCGTATCGACGACTGGCCGCCGGAACGCTCAGCGGCCGCGCGGTTGTGGTCCCTGGTCTGTGA
SEQ ID NO 155: Rhodococcus rhodochrous SCH80-06135 wt
MKAIQYTRIGAEPELTEIPKPEPGPGEVLLEVTAAGVCHSDDFIMSLPEEQYTYGLPLTLGHEGAGRVA
AVGEGVEGLDIGTNVVVYGPWGCGSCWHCSQGLENYCSRAKELGINPPGLGAPGALAEFMIVDSPRH
LVPIGDLDPVKTVPLTDAGLTPYHAIKRSLPKLRGGAYAVVIGTGGLGHVAIQLLRHLSAATVIALDVS
ADKLELATKVGAHEVVLSDKDAAENVRRITGSQGAALVLDFVGYQPTIDTAMAVAGVGSDVTIVGIG
DGQAHAKVGFFQSPYEASVTVPYWGARNELIELIDLAHAGIFDIAVETFSLDNGAEAYRRLAAGTLSGR
AVVVPGL*
SEQ ID NO 156: Rhodococcus rhodochrous SCH80-06135 E. coli optimized
ATGAAAGCAATCCAATATACCCGCATTGGTGCAGAGCCTGAGTTGACCGAGATCCCGAAACCGGA
ACCGGGTCCTGGCGAAGTTCTGCTCGAAGTTACCGCTGCGGGTGTGTGCCACAGCGATGACTTTAT
CATGTCGCTGCCAGAGGAACAATACACGTACGGCTTACCGCTGACGCTGGGTCATGAGGGCGCTG
GTCGTGTTGCAGCGGTGGGTGAGGGTGTCGAGGGCCTGGACATTGGCACCAACGTTGTCGTGTAC
GGTCCGTGGGGTTGCGGCTCTTGTTGGCATTGCTCCCAGGGCCTGGAGAATTACTGTTCCCGCGCG
AAAGAACTGGGTATCAATCCGCCTGGTCTGGGTGCTCCAGGTGCGCTGGCTGAGTTCATGATTGTC
GATAGCCCGCGTCACTTGGTTCCGATCGGTGACCTGGACCCGGTGAAAACCGTCCCGCTGACCGAT
GCGGGCTTGACGCCGTATCACGCGATTAAGCGCAGCCTGCCGAAACTGCGTGGTGGCGCGTATGC
AGTCGTCATCGGTACTGGTGGCTTGGGCCATGTTGCGATTCAGCTGCTGCGTCATCTGTCTGCCGC
GACGGTTATCGCGCTGGACGTGAGCGCCGATAAGCTCGAACTGGCCACTAAGGTTGGCGCGCACG
AAGTCGTTCTGAGCGATAAAGACGCAGCCGAAAATGTGCGTCGTATTACCGGTAGCCAGGGTGCA
GCATTGGTTCTGGACTTCGTTGGTTATCAGCCGACGATCGACACCGCGATGGCCGTTGCGGGCGTT
GGTAGCGATGTCACCATTGTGGGTATTGGCGATGGTCAAGCCCACGCCAAGGTTGGTTTCTTTCAA
AGCCCGTATGAAGCGAGCGTCACGGTGCCGTACTGGGGTGCGCGCAACGAACTGATCGAGCTGAT
CGATCTGGCTCACGCGGGTATTTTCGACATCGCAGTCGAAACCTTTAGCCTGGATAACGGCGCAGA
GGCATACCGTCGTCTGGCGGCTGGCACTCTGAGCGGTCGCGCAGTGGTAGTGCCGGGTCTGTAA
SEQ ID NO 157: Rhodococcus rhodochrous SCH80-06582 wt
ATGTTGGCAGTCCAGCTGACGGCGTGGGGTCAGCCTCCGCAGGTGCGTGAGATCCCCGTACCCGA
GCCCGCTGAGGGGCAACTGTTGATCAAAGTCGGCGCCGCTGGTCTGTGCCGCTCGGATCTGCACGT
CATGGATTCGCCCGCCGGACGTTTCGATTACCCGTTGCCGCTCACACTCGGCCATGAGGTTGCCGG
TACCGTCGTCGGTGCGGGACCGCTGGCCGATCACGCGTGGATCGGTGAAAATGTTGTCATTCATGG
TGTTTGGCCATGTGGCCGGTGCCGCAATTGCCGGCGCGAGCGCGAGAACTACTGCTTGGAGAAAG
TCCCGCGTGGGGACGGCCGACTCAGCCCGATCGGAAACGGGTTGGGCCATCCGGGCGGGCTGGCA
GAATACCTGCTGGTGCCCTCGGAAGCTGTTCTCGTTCGCGTCGGTTCGCTGAGCCCCCAGCAGGCC
GCTCCGCTCGCCGACGCCGGCCTGACCGCATATCATGCGATCCGGACCAACAGCGACGTCATCGA
CTCGGACACTGTGGCTTTGGTGATAGGAATCGGCGGTCTCGGCCATCTGGCGGTGCAGATCCTGCG
CTCTTTCGGCGTCACAGACATCATCGCCGTCGAGACAAGAACCCAGACACACGCTCTCGCGCTCGA
ATCGGGAGCACACGCGTGTTTTGCGACGCTCGCGGAAGCGACAGAGGCTGTGGCGAGCCTCGGCG
GTGCCGACGTGGTCTTCGACTTTGTCGGGGCTCAGGCGACGGTCGAACCCGCTCCGGCGCTTCTCG
CTCCCGGCGGCCGAGTTGTCGTCGTGGGAAGTGCGGGCGGGCAACTGACCGTCGGCAAAAGCCTT
GGTTTGGTCAACGGCTGGCAAGTTCGGGCGCCGTTCTGGGGCACCATCGAGGACCTGCGTCAGGT
GGTCGAACTCGCCAGTGCAGGAAAGCTGCATGCCGAGGTGACCACGTTCACGTTCGACAGCGCAC
TGGAGGCATACGATCGCCTGCGTTCAGGCGATCTGTCCGGCCGCGCCGTACTGGTTCCCACAGCCC
CTTCATCGCTGTGA
SEQ ID NO 158: Rhodococcus rhodochrous SCH80-06582 wt
MLAVQLTAWGQPPQVREIPVPEPAEGQLLIKVGAAGLCRSDLHVMDSPAGRFDYPLPLTLGHEVAGTV
VGAGPLADHAWIGENVVIHGVWPCGRCRNCRRERENYCLEKVPRGDGRLSPIGNGLGHPGGLAEYLL
VPSEAVLVRVGSLSPQQAAPLADAGLTAYHAIRTNSDVIDSDTVALVIGIGGLGHLAVQILRSFGVTDII
AVETRTQTHALALESGAHACFATLAEATEAVASLGGADVVFDFVGAQATVEPAPALLAPGGRVVVVG
SAGGQLTVGKSLGLVNGWQVRAPFWGTIEDLRQVVELASAGKLHAEVTTFTFDSALEAYDRLRSGDL
SGRAVLVPTAPSSL*
SEQ ID NO 159: Rhodococcus rhodochrous SCH80-06582 E. coli optimized
ATGTTAGCTGTTCAACTCACCGCATGGGGCCAACCACCACAAGTTCGCGAAATCCCGGTTCCGGAA
CCAGCCGAGGGCCAACTGCTGATTAAGGTTGGCGCAGCCGGTCTGTGCCGTAGCGACCTTCACGTT
ATGGACAGCCCTGCTGGTCGTTTTGATTACCCGTTGCCGCTGACGCTGGGTCACGAAGTGGCCGGC
ACGGTTGTCGGTGCCGGTCCGTTGGCAGACCACGCGTGGATTGGTGAGAACGTCGTGATTCACGGT
GTGTGGCCGTGTGGCCGTTGTCGTAATTGCCGTCGCGAGCGTGAGAACTACTGTTTGGAAAAAGTG
CCGCGTGGTGACGGTCGTCTGTCCCCGATCGGCAATGGTCTGGGTCATCCGGGTGGTCTGGCAGAG
TATCTGCTGGTGCCGAGCGAAGCCGTCCTGGTGCGTGTCGGCTCTCTGAGCCCGCAACAGGCAGCA
CCGCTGGCAGATGCGGGTCTGACCGCGTATCACGCGATTCGCACGAATAGCGACGTTATCGACTCT
GATACCGTGGCGCTGGTCATCGGTATTGGTGGCCTGGGTCACCTGGCCGTTCAGATTCTGCGTTCC
TTCGGCGTGACGGACATCATTGCAGTCGAGACTCGTACCCAGACGCATGCGTTGGCCCTGGAGAG
CGGTGCGCATGCGTGCTTTGCGACCCTGGCGGAAGCAACCGAAGCGGTTGCGAGCTTGGGCGGTG
CAGATGTTGTCTTTGACTTCGTTGGTGCGCAGGCGACTGTTGAGCCGGCACCAGCTCTGCTGGCAC
CTGGTGGCCGTGTTGTCGTGGTGGGTTCTGCGGGTGGCCAACTGACCGTCGGCAAATCCCTGGGTC
TGGTGAATGGCTGGCAAGTGCGTGCGCCGTTTTGGGGCACCATTGAAGATTTGCGTCAAGTCGTGG
AACTGGCGTCTGCAGGCAAGTTGCACGCCGAAGTTACCACGTTCACGTTCGATAGCGCGCTGGAA
GCGTACGACCGCCTGCGTAGCGGTGATCTGAGCGGTCGCGCTGTACTGGTTCCGACCGCCCCTAGC
AGCCTGTAA
SEQ ID NO 160: Rhodococcus erythropolis SCH94-03945 wt
ATGATTCGCGCCGAACAGAATTCGAGATCCTCCATGCAGATGACAGCGGCGCTCTCACACGGCCC
GCACTCCCCCTTCACGCTCGACACCGTCGAGATCGACGACCCCCGCGCAGACGAGATCCTGGTTCG
CATCGTCGCGACCGGCCTGTGCCACACAGATCTGTTCACGAAGTCGGCGCTACCGGAAAGACTCG
GCCCCTGCGTGTTCGGGCACGAAGGGGCGGGGGTGGTCGAGGCCGTCGGCTCGTCGATCGACAGC
ATTGCGCCCGGTGATCACGTGTTGCTGAGCTACCGCAGTTGCGGTGTGTGCAGGCAGTGTCTCAGC
GGCCATCGGGCGTACTGCGAAAGCTCACACGGGCTCAACAGCTCTGGCGCACGCACCGACGGCTC
GACGCCGATCCGGCGAGACGGAACCCCGCTACGGTCCGCCTTCTTCGGCCAGTCCAGCTTCGCGGA
ATACGTCATCGCCTCTGCCGACAACACCGTCGTCGTCGATCCTGCGGTGGACCTGACCGTCGCAGC
TCCGCTCGGCTGCGGGTTTCAAACCGGCGCCGGCGCGGTACTGAATCTGCTTCGCCCCGAGCCCGA
CTCGACGTTTGTCGTTTTCGGGGCAGGCAGCGTCGGACTCGCAGCGCTGCTGGCGGCGAGGGCTGC
CGGCGTTTCCACCCTGGTCGCCGTGGACCCCGTTGCGCAGCGGCGCGCACTCGCCGAGGAATTCGG
CGCCGTCACTGTCGATCCCTCGAATGAAGATGTGATCGACGCGGTCCACGCCGCCACCGACGGAG
GTTCGACGCATTCCCTCGACACCACCGGAATCGGCTCCGTGATCAATCAAGCCGTCACATCACTTC
GAGCACGGGGAACACTGGCGGTAGTCGGACTCGGAGCATCCACGGTCGAGGTGAACATGGCCGAC
ATCATGCTGAGCGGAAAGACAATTCGAGGATGCATCGAAGGAGAGTCGGAAGTCTCGACGTTCAT
CCCCGAACTCGTCGAACTCTTCACTGGTGGCCGGTTTCCGATCGACCGCTTGGTGACGCGCTACGC
ATTCGCCGACATCAACAAAGCCGTCGAAGATCAAGCGTCGGGGCGCGTCATCAAACCCGTTCTCG
TGTGGTGA
SEQ ID NO 161: Rhodococcus erythropolis SCH94-03945 wt
MIRAEQNSRSSMQMTAALSHGPHSPFTLDTVEIDDPRADEILVRIVATGLCHTDLFTKSALPERLGPCVF
GHEGAGVVEAVGSSIDSIAPGDHVLLSYRSCGVCRQCLSGHRAYCESSHGLNSSGARTDGSTPIRRDGT
PLRSAFFGQSSFAEYVIASADNTVVVDPAVDLTVAAPLGCGFQTGAGAVLNLLRPEPDSTFVVFGAGSV
GLAALLAARAAGVSTLVAVDPVAQRRALAEEFGAVTVDPSNEDVIDAVHAATDGGSTHSLDTTGIGS
VINQAVTSLRARGTLAVVGLGASTVEVNMADIMLSGKTIRGCIEGESEVSTFIPELVELFTGGRFPIDRL
VTRYAFADINKAVEDQASGRVIKPVLVW*
SEQ ID NO 162: Rhodococcus erythropolis SCH94-03945 E. coli optimized
ATGATTAGAGCAGAACAGAACAGCCGCAGCTCCATGCAAATGACCGCGGCACTGTCACATGGTCC
GCACAGCCCGTTTACGCTGGATACGGTTGAGATTGACGATCCACGCGCCGACGAAATTCTGGTACG
CATCGTTGCGACTGGTCTGTGTCATACGGACTTGTTTACCAAGAGCGCGCTGCCGGAGCGCCTGGG
TCCGTGCGTGTTCGGCCACGAGGGTGCGGGCGTGGTTGAGGCAGTTGGCTCTAGCATTGACAGCAT
CGCTCCGGGTGATCACGTCCTGTTGTCCTACCGTAGCTGCGGCGTCTGCCGTCAGTGCCTGAGCGG
CCACCGTGCTTACTGTGAGAGCTCCCACGGCCTGAATAGCTCCGGTGCTCGTACCGACGGTAGCAC
CCCGATCCGTCGTGATGGTACGCCGCTTCGTAGCGCGTTCTTCGGTCAATCCAGCTTCGCGGAATA
TGTTATCGCAAGCGCAGACAACACCGTTGTGGTCGATCCGGCCGTGGACTTGACCGTTGCAGCACC
GCTGGGTTGTGGCTTTCAGACCGGCGCCGGCGCGGTGCTGAATCTGCTGCGCCCTGAGCCGGACAG
CACTTTCGTCGTCTTTGGTGCCGGCAGCGTCGGTTTGGCGGCACTGCTGGCGGCGCGTGCGGCGGG
TGTTTCGACCCTGGTCGCAGTTGATCCGGTCGCGCAGCGCCGTGCGTTGGCCGAAGAATTTGGTGC
CGTTACCGTCGATCCGAGCAACGAAGATGTTATTGACGCTGTGCACGCGGCGACCGACGGTGGCA
GCACGCATTCTCTGGATACCACGGGCATCGGTTCTGTGATTAACCAAGCCGTGACCTCTCTGCGTG
CGCGTGGTACTCTGGCTGTGGTTGGCCTGGGTGCTAGCACGGTCGAGGTGAATATGGCAGACATTA
TGCTGAGCGGTAAAACGATCCGTGGTTGCATCGAGGGCGAGAGCGAAGTTTCGACGTTTATCCCG
GAACTGGTCGAGCTGTTCACCGGTGGCCGTTTCCCGATTGACCGCCTGGTTACCCGTTATGCATTC
GCCGATATCAACAAAGCTGTGGAAGATCAAGCGTCCGGTCGCGTCATCAAGCCAGTGCTGGTGTG
GTAA
SEQ ID NO 163: Rhodococcus rhodochrous SCH80-05240 wt
ATGATTCGCGCCGAACAGAATTCGACGTCCGCCATGCAGATGACAGCGGCGCTCTCACACGGCCC
GCACTCCCCCTTCACACTCGACACCGTCGAGATCGACGAACCCCGCGCAGACGAGATCCTGGTTCG
CATCGTCGCGACCGGCCTGTGCCACACAGATCTGTTCACGAAGTCGGTGCTACCGGAACGACTCGG
CCCCTGCGTGTTCGGGCACGAAGGGGCGGGGGTGGTCGAGGCCGTCGGCTCGGCGATCGACAAGG
TCGTGCCCGGCGATCACGTGTTGTTGAGCTACCGCAGTTGCGGTGTGTGCAGGCAGTGTCTCAGCG
GCCATCGGGCGTACTGCGAAAGCTCACACGGGCTCAACAGCTCTGGCGCACGCACCGACGGCTCG
ACGCCGGTCCGGCGAAGCGGAACTCCGATACGGTCCGCCTTCTTCGGCCAGTCCAGCTTCGCGGAA
TACGTCATCGCCACTGCCGACAACACCGTCGTCGTCGATCCTGCAGTGGACCTGACCGTCGCGGCT
CCCCTCGGCTGCGGATTTCAAACCGGCGCGGGTGCCGTGCTGAATCTACTTCGCCCCGAGCCCGAC
TCGACGTTTGTCGTCTTCGGAGCCGGCAGCGTCGGACTCGCAGCGCTACTGGCAGCGAGGGCTGCC
GGCGTTTCCACCCTGGTCGCCGTGGACCCCGTTGCGCAGCGGCGCGCACTCGCCGAGGAATTCGGC
GCCGTCACTGTCGATCCGACCACCGAGGACGCGGTCGAAGCAGTACGCGCCGCCACCGACGGAGG
TTCGACACATTCCCTCGACACCACCGGAATCGGCTCCGTGATCAATCAAGCCGTCACATCACTTCG
AGCACGGGGAACACTGGCGGTAGTCGGACTCGGAGCGTCCACGGTCGAGATGAACATGGCCGACA
TCATGCTGAGCGGAAAGACAATTCGAGGATGCATCGAAGGAGAGTCGGAAGTCTCGACGTTCATC
CCCGAACTCGTCGAACTCTTCACTGGTGGCCGGTTTCCGATCGACCGCTTGGTGACGCGCTACGCC
TTCTCCGACATCAACAAAGCCGTCGAAGATCAAGCGTCGGGGCGCGTCATCAAACCCGTTCTCGTG
TGGTGA
SEQ ID NO 164: Rhodococcus rhodochrous SCH80-05240 wt
MIRAEQNSTSAMQMTAALSHGPHSPFTLDTVEIDEPRADEILVRIVATGLCHTDLFTKSVLPERLGPCVF
GHEGAGVVEAVGSAIDKVVPGDHVLLSYRSCGVCRQCLSGHRAYCESSHGLNSSGARTDGSTPVRRS
GTPIRSAFFGQSSFAEYVIATADNTVVVDPAVDLTVAAPLGCGFQTGAGAVLNLLRPEPDSTFVVFGAG
SVGLAALLAARAAGVSTLVAVDPVAQRRALAEEFGAVTVDPTTEDAVEAVRAATDGGSTHSLDTTGI
GSVINQAVTSLRARGTLAVVGLGASTVEMNMADIMLSGKTIRGCIEGESEVSTFIPELVELFTGGRFPID
RLVTRYAFSDINKAVEDQASGRVIKPVLVW*
SEQ ID NO 165: Rhodococcus rhodochrous SCH80-05240 E. coli optimized
ATGATTAGAGCAGAACAGAACAGCACCAGCGCGATGCAAATGACCGCGGCACTGTCACATGGTCC
GCACAGCCCGTTTACGCTGGATACGGTTGAGATTGACGAGCCACGCGCCGACGAAATTCTGGTAC
GCATCGTTGCGACTGGTCTGTGTCATACGGACTTGTTTACCAAGAGCGTCCTGCCGGAGCGCCTGG
GTCCGTGCGTGTTCGGCCACGAGGGTGCGGGCGTGGTTGAGGCAGTTGGCTCTGCCATTGACAAA
GTTGTTCCGGGTGATCACGTCCTGTTGTCCTACCGTAGCTGCGGCGTCTGCCGTCAGTGCCTGAGC
GGCCACCGTGCTTACTGTGAGAGCTCCCACGGCCTGAATAGCTCCGGTGCTCGTACCGACGGTAGC
ACCCCGGTGCGTCGTAGCGGTACGCCGATTCGTAGCGCGTTCTTCGGTCAATCCAGCTTCGCGGAA
TATGTTATCGCAACCGCAGACAACACCGTTGTGGTCGATCCGGCCGTGGACTTGACCGTTGCAGCA
CCGCTGGGTTGTGGCTTTCAGACCGGCGCCGGCGCGGTGCTGAATCTGCTGCGCCCTGAGCCGGAC
AGCACTTTCGTCGTCTTTGGTGCCGGCAGCGTCGGTTTGGCGGCACTGCTGGCGGCGCGTGCGGCG
GGTGTTTCGACCCTGGTCGCAGTTGATCCGGTCGCGCAGCGCCGTGCGTTGGCCGAAGAATTTGGT
GCCGTTACCGTCGATCCGACGACCGAAGATGCCGTTGAAGCTGTGCGCGCGGCGACCGACGGTGG
CAGCACGCATTCTCTGGATACCACGGGCATCGGTTCTGTGATTAACCAAGCCGTGACCTCTCTGCG
TGCGCGTGGTACTCTGGCTGTGGTTGGCCTGGGTGCTAGCACGGTCGAGATGAATATGGCAGACAT
TATGCTGAGCGGTAAAACGATCCGTGGTTGCATCGAGGGCGAGAGCGAAGTTTCGACGTTTATCCC
GGAACTGGTCGAGCTGTTCACCGGTGGCCGTTTCCCGATTGACCGCCTGGTTACCCGTTATGCATT
CAGCGATATCAACAAAGCTGTGGAAGATCAAGCGTCCGGTCGCGTCATCAAGCCAGTGCTGGTGT
GGTAA
SEQ ID NO 166: Azoarcus toluclasticus AzTolADHI wt(NZ KB899498.1: 215502-
216629 (+))
ATGGGAAGCATCCAGGATTCGCTGTTCATTCGGGCACGCGCTGCCGTGCTGCGTACGGTGGGCGG
GCCGCTCGAGATCGAGAACGTGCGCATCAGCCCCCCCAAGGGCGATGAAGTGCTGGTGCGCATGG
TCGGAGTCGGCGTATGCCATACCGACGTGGTGTGCCGCGACGGTTTTCCCGTGCCGCTGCCGATTG
TGCTCGGGCACGAAGGCTCCGGCATCGTCGAGGCCGTCGGCGAGCGCGTGACGAAAGTGAAGCCG
GGCCAGCGTGTCGTGCTGTCGTTCAACTCCTGCGGGCACTGCGCGAGCTGCTGCGAGGATCACCCG
GCGACCTGCCACCAGATGCTGCCGCTCAACTTCGGCGCGGCGCAGCGCGTCGATGGGGGCACGGT
GATCGATGCGTCCGGCGAAGCGGTGCAGAGCCTCTTCTTCGGTCAGTCCTCGTTTGGCACCTACGC
GCTCGCGCGCGAAGTGAATACGGTCCCGGTTCCGGACGCCGTGCCGCTCGAAATCCTCGGCCCGCT
CGGTTGCGGGATCCAGACCGGGGCGGGTGCGGCGATCAATTCGCTCGCGCTGAAACCGGGCCAAT
CGCTCGCGATCTTCGGCGGGGGCAGCGTCGGCCTGAGCGCGCTGCTCGGCGCGCTCGCGGTCGGT
GCCGGCCCGGTGGTCGTGATTGAGCCCAACGAACGGCGTCGCGCGCTGGCGCTCGATCTGGGTGC
AAGCCACGCCTTCGATCCCTTCAACACCGAGGATCTCGTCGCGAGCATCAAGGCTGCGACCGGCG
GAGGCGTCACGCACTCGCTCGATTCGACGGGCCTCCCCCCCGTCATCGCCAACGCGATCAACTGCA
CCCTCCCGGGCGGCACCGTCGGCCTGCTGGGGGTGCCGTCACCCGAAGCCGCGGTGCCTGTGACCC
TGCTGGACCTGCTCGTGAAAAGCGTCACCCTGCGCCCGATCACCGAAGGCGACGCGAACCCGCAG
GAATTCATCCCGCGCATGGTCCAACTCTACCGCGACGGCAAGTTCCCCTTCGACAAGCTGATCACC
ACCTATCGCTTCGACGACATTAATCAAGCCTTCAAGGCGACCGAGACCGGAGAGGCGATCAAGCC
GGTGCTGGTGTTCTGA
SEQ ID NO 167: Azoarcus toluclasticus AzTolADH1 wt (WP_018990713.1)
MGSIQDSLFIRARAAVLRTVGGPLEIENVRISPPKGDEVLVRMVGVGVCHTDVVCRDGFPVPLPIVLGH
EGSGIVEAVGERVTKVKPGQRVVLSFNSCGHCASCCEDHPATCHQMLPLNFGAAQRVDGGTVIDASG
EAVQSLFFGQSSFGTYALAREVNTVPVPDAVPLEILGPLGCGIQTGAGAAINSLALKPGQSLAIFGGGSV
GLSALLGALAVGAGPVVVIEPNERRRALALDLGASHAFDPFNTEDLVASIKAATGGGVTHSLDSTGLPP
VIANAINCTLPGGTVGLLGVPSPEAAVPVTLLDLLVKSVTLRPITEGDANPQEFIPRMVQLYRDGKFPFD
KLITTYRFDDINQAFKATETGEAIKPVLVF*
SEQ ID NO 168: Azoarcus toluclasticus AzTolADH1 E. coli optimized
ATGGGTTCTATTCAAGATTCTCTGTTCATCCGTGCACGCGCCGCTGTTCTGCGTACTGTCGGTGGCC
CGCTGGAAATTGAAAACGTCCGCATTAGCCCTCCGAAGGGTGACGAAGTGCTCGTGCGTATGGTT
GGTGTTGGTGTGTGCCATACCGACGTTGTGTGTCGCGATGGCTTCCCGGTTCCGCTGCCGATTGTGC
TGGGTCACGAGGGCAGCGGTATTGTCGAGGCAGTGGGCGAGCGTGTGACCAAGGTTAAACCGGGT
CAGCGTGTCGTTTTATCCTTCAATAGCTGTGGTCATTGCGCGTCCTGCTGCGAGGACCACCCGGCC
ACCTGTCACCAGATGCTGCCACTGAACTTTGGTGCGGCGCAGCGCGTGGATGGTGGCACCGTTATC
GACGCGAGCGGCGAGGCAGTGCAGAGCCTGTTTTTTGGTCAAAGCTCTTTCGGTACGTATGCATTG
GCGCGTGAAGTCAATACCGTACCGGTGCCGGATGCAGTTCCGTTGGAAATCCTGGGCCCGTTGGGT
TGCGGCATCCAGACGGGTGCGGGTGCGGCTATCAACAGCCTGGCGCTGAAACCTGGTCAATCGCT
GGCAATCTTCGGTGGCGGCAGCGTCGGTCTGTCCGCCCTGCTGGGCGCGCTGGCCGTGGGCGCGG
GCCCGGTCGTTGTCATTGAGCCGAACGAACGTCGTCGTGCGTTGGCGCTGGACCTGGGTGCGAGCC
ATGCATTTGATCCGTTCAACACTGAAGATTTGGTTGCGAGCATCAAAGCCGCTACGGGTGGCGGCG
TTACCCACAGCCTGGACAGCACGGGTCTGCCGCCGGTCATCGCGAATGCAATCAACTGTACCTTGC
CGGGCGGCACGGTCGGTCTGCTGGGCGTCCCGAGCCCAGAGGCTGCCGTTCCGGTGACGCTGCTG
GATCTGCTGGTTAAATCAGTTACCCTGCGTCCGATTACCGAGGGTGACGCCAATCCGCAAGAATTT
ATTCCGCGTATGGTCCAGCTGTACCGCGACGGTAAATTTCCGTTTGATAAGCTGATTACGACCTAC
CGCTTCGACGACATCAATCAAGCGTTCAAGGCAACCGAAACCGGTGAAGCGATTAAGCCAGTGCT
GGTGTTTTAA
SEQ ID NO 169: Aspersillus wentii AspWeTPP wt (KV878213.1: 2482776-2483627)
ATGGCATCTGTACCAGCTCCCCCATTTGTCCACGTCGAAGGAATGAGCAATTTCCGATCGATAGGA
GGATATCCCCTTGAGACAGCATCGACAAACAATCACCGCTCCACGAGGCAAGGATTCGCATTTCG
CAGTGCCGATCCAACCTACGTCACCCAGAAAGGCCTGGAAACCATCCTTTCGCTCGACATCACTCG
AGCCTTTGACCTCCGCTCACTGGAAGAAGCAAAGGCACAGCGCGCAAAACTCCAGGCCGCCTCAG
GATGTCTCGACTGCAGCATCAGCCAGCACATGATCCACCAGCCCACACCCCTATTTCCAGATGGGG
ACTGGAGTCCAGAGGCCGCAGGGGAGCGGTATCTGCAGTACGCCCAGGCTGAGGGAGATGGGAT
ATCGGGCTACGTGGAGGTCTACGGAAACATGCTCGAGGAAGGTTGGATGGCGATTCGCGAGATTC
TGCTTCATGTCCGGGACCGGCCTACAGAGGCGTTTCTATGCCATTGTAGTGCAGGGAAAGATCGTA
CGGGGATTGTCATTGCGGTTTTGTTGAAGGTTGCAGGGTGCTCGGATGATCTTGTGTGCAGAGAGT
ATGAGTTGACCGAGATCGGGTTGGCTCGACGGAGGGAGTTTATCGTGCAGCATCTGCTTAAGAAG
CCGGAAATGAATGGATCGAGGGAACTGGCCGAAAGAGTGGCGGGGGCCAGGTATGAGAATATGA
AGGAAACGCTGGAGATGGTGCAAACTAGATATAGAGGGATGAGGGGCTATTGCAAGGAGATTTGC
GGCTTGACCGACGAAGATCTATCTATTATCCAGGGGAACTTGACTAGTCCGGAGAGTCCTATCTTC
TAA
SEQ ID NO 170: Aspersillus wentii AspWeTPP wt (OJJ34585.1)
MASVPAPPFVHVEGMSNFRSIGGYPLETASTNNHRSTRQGFAFRSADPTYVTQKGLETILSLDITRAFDL
RSLEEAKAQRAKLQAASGCLDCSISQHMIHQPTPLFPDGDWSPEAAGERYLQYAQAEGDGISGYVEVY
GNMLEEGWMAIREILLHVRDRPTEAFLCHCSAGKDRTGIVIAVLLKVAGCSDDLVCREYELTEIGLARR
REFIVQHLLKKPEMNGSRELAERVAGARYENMKETLEMVQTRYRGMRGYCKEICGLTDEDLSIIQGNL
TSPESPIF
SEQ ID NO 171: Aspergillus wentii AspWeTPP E. coli optimized
ATGGCGTCTGTCCCTGCTCCACCGTTTGTTCATGTTGAAGGTATGTCTAATTTTCGTAGCATCGGTG
GCTACCCGCTGGAGACTGCCTCCACGAATAACCATCGCTCGACCCGTCAAGGCTTCGCGTTTCGTA
GCGCGGACCCGACGTATGTGACGCAGAAAGGCCTGGAAACCATTCTGTCCCTGGATATTACCCGC
GCATTTGACTTGCGTAGCTTGGAAGAAGCAAAGGCACAACGTGCGAAGTTGCAGGCCGCGAGCGG
TTGTCTGGATTGCAGCATTAGCCAACACATGATCCACCAACCGACCCCGCTGTTCCCGGATGGTGA
CTGGTCCCCGGAAGCGGCGGGTGAGCGCTACTTGCAGTACGCACAAGCTGAGGGTGATGGTATCA
GCGGTTATGTCGAAGTTTATGGTAATATGCTGGAAGAGGGCTGGATGGCGATCCGTGAGATTCTGC
TGCACGTCCGTGACCGCCCGACCGAAGCATTCCTGTGCCACTGTTCCGCCGGTAAAGATCGTACGG
GTATCGTGATTGCTGTTCTGCTCAAAGTCGCGGGTTGCAGCGACGACCTGGTGTGTCGTGAGTACG
AACTGACCGAGATTGGCCTGGCGCGCCGTAGAGAGTTCATCGTTCAGCATCTGCTGAAGAAACCG
GAAATGAACGGCAGCCGTGAGCTGGCGGAGCGCGTCGCAGGCGCCCGTTACGAGAACATGAAAG
AAACCCTGGAAATGGTGCAGACCCGTTACCGCGGCATGCGCGGCTATTGCAAAGAAATCTGCGGT
CTGACCGACGAAGATCTGAGCATTATCCAGGGTAACCTGACGAGCCCGGAGAGCCCGATTTTCTA
A
SEQ ID NO 172: Talaromyces verruculosus PvCPS (LC316181.1)
ATGAGCCCAATGGATTTACAAGAATCAGCGGCAGCTTTGGTGCGGCAGTTGGGGGAGAGAGTCGA
AGATCGCCGTGGTTTTGGATTCATGAGCCCTGCCATCTATGATACCGCATGGGTCTCTATGATTAG
CAAGACAATCGATGACCAAAAAACATGGTTGTTTGCAGAATGTTTCCAGTACATTCTTTCTCATCA
GCTCGAAGACGGTGGTTGGGCAATGTATGCATCTGAAATCGACGCCATCCTAAACACTTCGGCCTC
ATTACTATCATTAAAGAGACATCTTTCAAATCCCTATCAAATTACATCTATCACACAAGAGGATCT
GTCCGCCCGCATTAACAGGGCTCAGAATGCTTTACAGAAGCTTCTCAATGAGTGGAATGTCGACAG
CACGCTCCACGTGGGATTCGAGATCCTAGTTCCGGCCCTACTCAGGTATCTCGAAGATGAGGGCAT
CGCTTTTGCTTTTTCTGGTAGAGAGCGCCTGCTTGAGATTGAGAAACAGAAATTATCAAAGTTCAA
AGCACAGTATCTATACCTTCCAATCAAAGTGACAGCTTTGCATTCTCTGGAAGCGTTCATAGGCGC
CATTGAGTTTGATAAAGTCAGTCACCACAAAGTCAGCGGTGCGTTCATGGCATCTCCATCATCCAC
AGCAGCTTACATGATGCATGCGACACAATGGGATGATGAATGCGAGGATTACCTACGCCACGTCA
TTGCTCATGCATCTGGGAAAGGATCCGGAGGTGTTCCAAGCGCTTTTCCTTCCACCATCTTTGAAA
GCGTTTGGCCTCTATCAACTCTGCTAAAGGTGGGATATGATCTCAACTCGGCACCTTTTATCGAAA
AAATCAGATCATACTTGCATGATGCATATATTGCTGAAAAGGGAATTCTCGGCTTCACTCCTTTTGT
TGGCGCTGATGCAGATGATACCGCTACCACCATATTGGTGCTCAATCTTTTGAACCAACCAGTCTC
AGTCGACGCGATGTTGAAGGAATTTGAAGAAGAACATCACTTCAAAACCTACTCTCAGGAGCGCA
ATCCTAGTTTCTCGGCCAATTGTAACGTTCTTCTTGCCTTACTATACAGTCAAGAGCCATCGCTTTA
TAGCGCGCAGATCGAAAAAGCTATAAGGTTCCTCTATAAGCAATTCACAGATTCAGAAATGGACG
TTCGAGACAAATGGAATCTATCACCATACTATTCTTGGATGCTCATGACACAAGCCATCACGCGGT
TGACGACTCTTCAGAAGACTTCGAAACTTTCAACATTGAGAGATGATTCTATCAGCAAAGGCTTGA
TTAGTCTGCTGTTTAGGATAGCTTCTACCGTGGTTAAAGACCAAAAGCCAGGAGGTTCTTGGGGCA
CTCGAGCTTCGAAAGAAGAGACTGCCTACGCAGTGTTGATTCTCACATATGCTTTCTACCTCGATG
AGGTTACGGAGTCGTTGCGGCATGATATCAAGATCGCCATTGAGAATGGTTGCTCATTCCTATCTG
AAAGAACCATGCAGTCCGATTCGGAGTGGCTTTGGGTTGAGAAAGTCACATATAAATCAGAGGTT
CTTTCGGAAGCATATATCTTGGCCGCTCTTAAACGGGCAGCTGACTTACCCGACGAAAATGCAGAA
GCAGCCCCCGTCATAAATGGAATTTCTACAAATGGATTTGAGCATACCGATAGAATTAACGGCAA
GCTTAAAGTCAATGGTACCAACGGTACAAATGGCAGTCATGAGACAAACGGTATCAACGGTACGC
ATGAAATTGAACAGATCAATGGCGTCAACGGCACGAATGGTCACTCTGATGTGCCTCACGATACA
AATGGCTGGGTAGAAGAGCCGACCGCCATCAATGAGACAAATGGCCACTACGTGAATGGCACGAA
TCACGAGACTCCCCTTACCAACGGCATTTCCAATGGAGATTCTGTTTCCGTTCATACAGACCACTC
GGACAGTTACTATCAGCGCAGTGATTGGACAGCCGACGAAGAACAAATTCTTCTCGGTCCATTTGA
CTACCTGGAGAGCCTGCCAGGCAAGAATATGCGCTCACAACTGATTCAATCATTCAACACATGGCT
CAAAGTCCCAACTGAGAGCTTGGATGTTATTATTAAGGTGATTTCAATGTTGCATACGGCCTCTCT
CTTGATCGATGATATTCAGGATCAATCAATACTCCGCCGCGGGCAACCTGTAGCGCACAGCATCTT
TGGCACAGCGCAAGCAATGAACTCAGGGAATTATGTCTACTTTCTAGCCCTTAGGGAGGTTCAGAA
ACTACAAAACCCGAAAGCCATCAGTATTTATGTTGACTCTTTGATTGATCTTCACCGTGGCCAAGG
CATGGAGCTTTTCTGGCGGGATTCTCTCATGTGCCCAACCGAAGAGCAGTACCTTGACATGGTCGC
AAACAAAACTGGCGGCCTGTTTTGCCTTGCTATCCAATTGATGCAAGCTGAAGCCACTATCCAAGT
CGACTTCATACCACTTGTCCGACTACTCGGCATCATCTTCCAGATTTGTGATGATTACTTGAATCTG
AAGTCTACGGCCTATACAGACAACAAAGGGTTGTGTGAGGATTTGACAGAGGGCAAATTCTCTTTT
CCTATCATCCATAGCATTCGATCCAACCCTGGCAACCGACAGCTAATCAACATCTTGAAGCAAAAG
CCACGTGAAGACGACATCAAACGCTATGCTCTATCCTATATGGAAAGCACCAACTCATTTGAGTAT
ACTCGGGGTGTCGTTAGAAAACTGAAGACCGAGGCAATCGATACTATTCAAGGCTTGGAGAAGCA
CGGCCTGGAAGAGAATATTGGCATTCGAAAGATACTAGCTCGCATGTCCCTTGAGCTATGA
SEQ ID NO 173: Talaromyces verruculosus PvCPS (BBF88128.1)
MSPMDLQESAAALVRQLGERVEDRRGFGFMSPAIYDTAWVSMISKTIDDQKTWLFAECFQYILSHQLE
DGGWAMYASEIDAILNTSASLLSLKRHLSNPYQITSITQEDLSARINRAQNALQKLLNEWNVDSTLHVG
FEILVPALLRYLEDEGIAFAFSGRERLLEIEKQKLSKFKAQYLYLPIKVTALHSLEAFIGAIEFDKVSHHK
VSGAFMASPSSTAAYMMHATQWDDECEDYLRHVIAHASGKGSGGVPSAFPSTIFESVWPLSTLLKVGY
DLNSAPFIEKIRSYLHDAYIAEKGILGFTPFVGADADDTATTILVLNLLNQPVSVDAMLKEFEEEHHFKT
YSQERNPSFSANCNVLLALLYSQEPSLYSAQIEKAIRFLYKQFTDSEMDVRDKWNLSPYYSWMLMTQA
ITRLTTLQKTSKLSTLRDDSISKGLISLLFRIASTVVKDQKPGGSWGTRASKEETAYAVLILTYAFYLDEV
TESLRHDIKIAIENGCSFLSERTMQSDSEWLWVEKVTYKSEVLSEAYILAALKRAADLPDENAEAAPVI
NGISTNGFEHTDRINGKLKVNGTNGTNGSHETNGINGTHEIEQINGVNGTNGHSDVPHDTNGWVEEPT
AINETNGHYVNGTNHETPLTNGISNGDSVSVHTDHSDSYYQRSDWTADEEQILLGPFDYLESLPGKNM
RSQLIQSFNTWLKVPTESLDVIIKVISMLHTASLLIDDIQDQSILRRGQPVAHSIFGTAQAMNSGNYVYFL
ALREVQKLQNPKAISIYVDSLIDLHRGQGMELFWRDSLMCPTEEQYLDMVANKTGGLFCLAIQLMQAE
ATIQVDFIPLVRLLGIIFQICDDYLNLKSTAYTDNKGLCEDLTEGKFSFPIIHSIRSNPGNRQLINILKQKPR
EDDIKRYALSYMESTNSFEYTRGVVRKLKTEAIDTIQGLEKHGLEENIGIRKILARMSLEL
SEQ ID NO 174: Talaromyces verruculosus PvCPS E. coli optimized
ATGAGCCCTATGGATTTGCAAGAAAGCGCCGCAGCCCTGGTCCGTCAATTGGGTGAACGCGTTGA
GGATCGCCGCGGTTTTGGTTTCATGAGCCCGGCCATTTATGACACGGCCTGGGTTAGCATGATTAG
CAAGACCATCGACGACCAAAAAACTTGGCTGTTTGCGGAGTGCTTCCAGTACATTCTGTCTCACCA
ACTGGAAGATGGTGGCTGGGCGATGTACGCATCCGAAATCGATGCCATCTTGAATACTTCCGCGTC
ACTGCTGTCCCTGAAACGCCACCTGTCCAACCCTTACCAGATCACCAGCATCACTCAGGAAGATCT
GAGCGCTCGCATCAACCGCGCTCAAAACGCCCTGCAGAAATTGCTGAACGAGTGGAACGTTGACT
CCACGCTGCACGTCGGTTTCGAGATTCTGGTTCCGGCGCTGCTGCGCTATCTGGAAGATGAAGGCA
TCGCGTTTGCGTTCTCGGGTCGTGAGCGTTTGTTAGAGATTGAGAAACAAAAACTGTCCAAGTTTA
AAGCGCAGTATTTGTACTTACCGATTAAGGTCACCGCACTGCATAGCCTGGAAGCCTTCATCGGCG
CTATTGAGTTCGACAAAGTCAGCCATCACAAAGTATCCGGTGCTTTCATGGCGTCGCCGTCTAGCA
CCGCAGCATACATGATGCATGCGACGCAATGGGATGACGAATGTGAGGATTACTTGCGTCACGTG
ATCGCGCATGCGTCAGGTAAGGGTTCTGGCGGCGTGCCGAGCGCCTTTCCGAGCACCATCTTCGAG
AGCGTTTGGCCGCTGTCTACTCTGCTGAAAGTTGGCTATGATCTGAATAGCGCTCCGTTCATCGAG
AAAATTCGTAGCTACTTGCACGATGCCTATATCGCAGAGAAAGGTATTCTCGGTTTCACCCCGTTC
GTTGGCGCTGACGCGGACGACACCGCTACCACGATTCTGGTGTTGAATCTGCTGAACCAACCGGTG
AGCGTGGACGCGATGTTGAAAGAATTTGAAGAGGAACATCACTTCAAGACCTACAGCCAAGAGCG
TAATCCGAGCTTTTCCGCAAACTGTAATGTTCTGCTGGCGCTGCTGTACAGCCAGGAACCGAGCCT
GTACAGCGCGCAAATCGAAAAAGCGATCCGTTTTCTGTATAAGCAATTCACCGACTCTGAGATGG
ATGTGCGCGATAAATGGAACCTGTCCCCGTATTATAGCTGGATGCTGATGACCCAGGCCATCACCC
GTCTGACGACCCTGCAAAAGACCAGCAAGCTGAGCACGCTGCGTGATGACAGCATTAGCAAGGGC
CTGATTTCTCTGCTGTTCCGCATTGCATCCACCGTGGTTAAAGATCAAAAACCGGGTGGCAGCTGG
GGCACGCGTGCGAGCAAAGAAGAAACGGCATACGCCGTGCTGATTCTGACCTACGCGTTTTATCT
GGACGAGGTGACCGAGTCTCTGCGCCACGATATCAAAATTGCAATCGAGAATGGTTGCTCGTTCCT
GAGCGAGCGCACCATGCAAAGCGACAGCGAGTGGCTGTGGGTCGAAAAGGTTACCTACAAGAGC
GAAGTGCTGAGCGAAGCATACATCCTGGCAGCTCTGAAACGTGCGGCAGACTTGCCGGATGAGAA
CGCTGAGGCAGCCCCAGTGATCAACGGTATCTCTACCAATGGCTTTGAGCACACCGACCGCATTAA
TGGTAAACTCAAGGTCAATGGTACGAATGGCACCAACGGTTCCCACGAAACGAACGGTATCAATG
GCACCCATGAGATTGAGCAAATTAATGGTGTCAACGGCACGAATGGCCATAGCGACGTGCCACAT
GACACGAATGGTTGGGTCGAGGAACCGACGGCGATTAATGAAACGAACGGTCACTACGTTAACGG
CACCAACCATGAGACTCCGCTGACCAATGGTATTAGCAATGGTGACTCCGTGAGCGTTCACACCGA
CCATAGCGACAGCTACTATCAGCGTAGCGACTGGACCGCGGATGAAGAACAGATCCTGCTGGGTC
CATTCGATTACCTGGAATCCCTGCCTGGTAAAAATATGCGCAGCCAGCTGATCCAGTCTTTCAATA
CGTGGCTGAAGGTCCCGACCGAGAGCTTGGACGTGATTATTAAGGTCATTAGCATGCTGCACACTG
CTAGCCTGCTGATCGACGATATTCAGGACCAAAGCATCCTGCGTCGTGGTCAGCCTGTGGCGCACT
CGATCTTCGGCACCGCGCAAGCGATGAACTCTGGTAACTATGTTTACTTCCTGGCATTGCGTGAAG
TTCAGAAATTGCAAAACCCGAAGGCTATCAGCATTTATGTGGACAGCTTGATCGATCTTCATCGCG
GCCAGGGCATGGAACTGTTCTGGCGTGATTCTCTGATGTGCCCGACTGAAGAACAGTATCTGGACA
TGGTGGCGAACAAGACCGGTGGCCTGTTTTGTCTGGCGATTCAGCTGATGCAGGCAGAAGCGACC
ATTCAGGTTGATTTTATTCCGCTGGTGCGTCTGCTGGGTATCATTTTCCAGATTTGCGACGACTACC
TGAACTTGAAAAGCACTGCGTATACCGACAACAAAGGTCTGTGTGAAGATCTTACCGAGGGTAAA
TTCTCCTTCCCGATCATTCACAGCATCCGTAGCAATCCGGGCAATCGTCAGCTGATCAATATTCTGA
AGCAAAAACCGCGCGAAGATGACATCAAGCGTTACGCACTGTCCTATATGGAGAGCACGAATAGC
TTCGAGTACACCCGTGGCGTCGTCCGTAAATTGAAAACCGAAGCAATTGACACGATTCAAGGTCTG
GAGAAGCATGGCCTGGAAGAAAACATTGGTATTCGTAAGATTCTGGCGCGTATGAGCCTGGAACT
GTAA
SEQ ID NO 175: Talaromyces cellulolyticus TalCeTPP wt
ATGTCTAATGACACCACTAGCACGGCTTCTGCCGGAACAGCAACTTCTTCGCGGTTTCTTTCTGTGG
GCGGAGTTGTGAATTTCCGTGAACTGGGCGGTTATCCATGTGATTCTGTCCCTCCTGCTCCTGCCTC
AAACGGCTCACCGGACAACGCATCTGAAGCGATCCTTTGGGTTGGCCACTCGTCCATTCGGCCTAG
GTTTCTCTTTCGATCGGCACAGCCGTCTCAGATTACCCCGGCCGGTATTGAGACATTGATCCGCCA
GCTTGGCATCCAGGCAATTTTTGACTTTCGTTCACGGACGGAAATTCAGCTTGTCGCCACTCGCTAT
CCTGATTCGCTACTCGAGATACCTGGTACGACTCGCTATTCCGTGCCCGTCTTCACGGAGGGCGAC
TATTCCCCGGCGTCATTAGTCAAGAGGTACGGAGTGTCCTCCGATACTGCAACTGATTCCACTTCC
TCCAAATGTGCCAAGCCTACAGGATTCGTCCACGCATATGAGGCTATCGCACGCAGCGCAGCAGA
AAACGGCAGTTTTCGTAAAATAACGGACCACATAATACAACATCCGGACCGGCCTATCCTGTTTCA
CTGTACATTGGGAAAAGACCGAACCGGTGTATTTGCAGCATTGTTATTGAGTCTTTGCGGGGTACC
AAACGACACGATAGTTGAAGACTATGCTATGACTACCGAGGGATTTGGGGTCTGGCGAGAACATC
TAATTCAACGCCTGTTACAAAGAAAGGATGCAGCTACGCGTGAGGATGCAGAATTCATTATTGCC
AGCCACCCGGAGAGTATGAAGGCTTTTCTAGAAGATGTGGTAGCAACCAAGTTCGGGGATGCTCG
AAATTACTTTATCCAGCACTGTGGATTGACGGAAGCGGAGGTTGATAAGCTAATTCGGACACTGGT
CATTGCGAATTGA
SEQ ID NO 176: Talaromyces cellulolyticus TalCeTPP wt (GAM42000.1)
MSNDTTSTASAGTATSSRFLSVGGVVNFRELGGYPCDSVPPAPASNGSPDNASEAILWVGHSSIRPRFLF
RSAQPSQITPAGIETLIRQLGIQAIFDFRSRTEIQLVATRYPDSLLEIPGTTRYSVPVFTEGDYSPASLVKRY
GVSSDTATDSTSSKCAKPTGFVHAYEAIARSAAENGSFRKITDHIIQHPDRPILFHCTLGKDRTGVFAAL
LLSLCGVPNDTIVEDYAMTTEGFGVWREHLIQRLLQRKDAATREDAEFIIASHPESMKAFLEDVVATKF
GDARNYFIQHCGLTEAEVDKLIRTLVIAN
SEQ ID NO 177: Talaromyces cellulolyticus TalCeTPP E. coli optimized
ATGAGCAACGACACGACCAGCACCGCATCCGCAGGCACCGCAACTTCTTCGCGCTTTCTGAGCGTC
GGTGGCGTGGTTAACTTCCGTGAGTTGGGTGGCTACCCGTGCGACAGCGTTCCTCCTGCACCAGCA
AGCAATGGTAGCCCGGACAATGCGAGCGAAGCGATTCTGTGGGTTGGTCACAGCAGCATTCGTCC
GCGCTTCTTGTTTCGTAGCGCACAGCCGTCCCAGATCACCCCGGCCGGTATTGAAACGCTGATTCG
CCAACTCGGTATTCAAGCGATCTTTGACTTTCGTTCCCGTACCGAGATCCAACTGGTGGCAACCCG
CTACCCAGATAGCCTGCTGGAAATTCCGGGCACGACTCGTTACTCTGTTCCGGTCTTTACCGAGGG
CGACTACAGCCCGGCTTCTCTGGTTAAGCGTTATGGTGTCTCTAGCGACACGGCAACGGATAGCAC
CAGCTCAAAGTGCGCGAAACCGACCGGCTTTGTGCATGCTTATGAAGCGATTGCTCGTTCTGCCGC
GGAGAACGGTAGCTTCCGCAAGATCACCGACCACATTATCCAACATCCGGATCGCCCGATCCTGTT
TCACTGCACGCTGGGCAAAGACCGTACCGGTGTTTTCGCAGCGCTGCTGCTGAGCTTGTGTGGTGT
CCCGAATGACACCATCGTGGAAGATTATGCGATGACGACCGAAGGCTTCGGTGTGTGGCGTGAGC
ACTTGATTCAGCGTCTGCTGCAGCGCAAAGATGCGGCTACGCGTGAAGATGCCGAGTTCATTATCG
CGAGCCATCCGGAGAGCATGAAAGCGTTCCTGGAAGATGTCGTTGCGACCAAATTCGGTGACGCC
CGCAACTACTTTATCCAGCACTGTGGTCTGACCGAAGCCGAAGTGGATAAGCTGATCCGTACGCTG
GTGATCGCGAATTAA
SEQ ID NO 178: Castellaniella defrasrans CdGeoA wt (NZ HG916765.1 3061533-
3062654 (+))
ATGAACGACACCCAGGATTTCATTTCCGCGCAGGCCGCCGTGCTGCGCCAGGTCGGCGGGCCGCTC
GCGGTCGAGCCCGTGCGCATCAGCATGCCCAAAGGCGACGAGGTCTTGATCCGCATCGCCGGCGT
GGGCGTCTGCCACACCGACCTGGTGTGCCGCGACGGATTTCCCGTGCCGCTGCCGATCGTGCTCGG
CCACGAAGGCTCCGGCACCGTCGAGGCGGTGGGCGAGCAGGTGCGCACGCTCAAGCCCGGCGACC
GGGTCGTGCTGTCCTTCAATTCCTGCGGGCATTGCGGCAATTGCCACGACGGCCATCCGTCGAACT
GCCTGCAGATGCTGCCCCTGAACTTCGGCGGCGCGCAGCGCGTGGACGGCGGCCAGGTGCTGGAC
GGCGCCGGCCATCCCGTGCAGAGCATGTTCTTCGGCCAGTCCTCGTTCGGCACGCATGCCGTGGCG
CGCGAAATCAATGCGGTCAAGGTCGGCGACGACCTGCCGCTGGAACTGCTGGGCCCGCTGGGCTG
CGGCATCCAGACCGGCGCGGGCGCGGCGATCAATTCGCTGGGGATCGGCCCGGGCCAGTCCCTGG
CCATCTTCGGCGGTGGCGGCGTCGGCCTGAGCGCGCTGCTGGGCGCGCGCGCCGTCGGGGCGGAC
CGGGTCGTGGTGATCGAGCCCAATGCCGCGCGCCGGGCCCTGGCCCTGGAACTGGGCGCCAGCCA
TGCCCTCGACCCGCACGCCGAAGGCGACCTGGTGGCCGCGATCAAGGCGGCCACCGGCGGCGGCG
CGACCCACTCGCTGGACACGACGGGCCTGCCCCCGGTCATCGGCAGCGCGATCGCCTGCACCCTGC
CGGGCGGCACCGTGGGCATGGTCGGACTGCCGGCGCCCGATGCCCCGGTGCCGGCGACCCTGCTC
GATCTGCTGAGCAAAAGCGTCACCCTGCGCCCGATCACCGAGGGCGACGCGGACCCGCAGCGCTT
CATCCCGCGCATGCTGGATTTCCATCGCGCGGGCAAATTCCCGTTCGACCGGCTGATCACCCGCTA
CCGTTTCGACCAGATCAACGAGGCCCTGCACGCCACCGAGAAGGGCGAGGCGATCAAGCCGGTGC
TGGTGTTCTGA
SEQ ID NO 179: Castellaniella defrasrans CdGeoA wt (WP_043683915.1)
MNDTQDFISAQAAVLRQVGGPLAVEPVRISMPKGDEVLIRIAGVGVCHTDLVCRDGFPVPLPIVLGHEG
SGTVEAVGEQVRTLKPGDRVVLSFNSCGHCGNCHDGHPSNCLQMLPLNFGGAQRVDGGQVLDGAGH
PVQSMFFGQSSFGTHAVAREINAVKVGDDLPLELLGPLGCGIQTGAGAAINSLGIGPGQSLAIFGGGGV
GLSALLGARAVGADRVVVIEPNAARRALALELGASHALDPHAEGDLVAAIKAATGGGATHSLDTTGL
PPVIGSAIACTLPGGTVGMVGLPAPDAPVPATLLDLLSKSVTLRPITEGDADPQRFIPRMLDFHRAGKFP
FDRLITRYRFDQINEALHATEKGEAIKPVLVF
SEQ ID NO 180: Castellaniella defrasrans CdGeoA E. coli optimized
ATGAACGATACGCAGGATTTTATTAGCGCCCAAGCCGCAGTGTTACGTCAGGTCGGTGGCCCGCTG
GCCGTTGAGCCTGTTCGTATCAGCATGCCGAAGGGTGACGAAGTCCTGATTCGTATCGCGGGTGTT
GGTGTGTGCCACACCGACTTGGTGTGCCGTGATGGCTTCCCGGTGCCGCTGCCAATTGTGCTGGGT
CACGAGGGTAGCGGTACTGTCGAAGCCGTCGGTGAACAAGTCCGTACCCTGAAACCGGGCGATCG
CGTCGTGCTGAGCTTTAACAGCTGCGGTCATTGCGGTAACTGTCACGACGGTCACCCGAGCAATTG
CCTGCAGATGCTGCCGCTGAACTTCGGTGGCGCGCAACGCGTGGACGGTGGCCAAGTTTTGGACG
GTGCGGGTCATCCGGTTCAGTCCATGTTTTTCGGCCAGTCCAGCTTTGGCACCCACGCAGTAGCGC
GCGAGATCAACGCAGTCAAGGTCGGCGATGATCTGCCACTGGAACTGCTGGGTCCGTTGGGTTGT
GGCATTCAAACCGGTGCGGGTGCAGCTATCAATTCTCTGGGCATTGGTCCGGGTCAGTCTCTGGCT
ATCTTCGGCGGCGGCGGCGTGGGTCTGAGCGCACTGCTGGGCGCCCGTGCGGTGGGTGCCGACCG
TGTTGTTGTCATTGAGCCGAATGCAGCGCGCCGTGCGCTGGCATTGGAACTGGGTGCCAGCCACGC
ACTGGACCCGCATGCCGAGGGCGACCTTGTTGCGGCGATTAAAGCTGCGACGGGTGGCGGCGCTA
CGCATAGCTTGGATACGACCGGCCTGCCGCCAGTCATTGGCTCCGCGATCGCGTGTACTCTGCCGG
GTGGCACCGTTGGTATGGTTGGTCTGCCGGCGCCGGACGCACCGGTCCCTGCGACGCTGTTGGATC
TGCTGAGCAAATCGGTTACCCTGCGTCCGATTACCGAGGGTGACGCTGACCCGCAACGCTTCATCC
CGCGTATGCTGGATTTCCATCGTGCGGGCAAGTTTCCGTTCGACCGCCTGATCACCCGTTACCGCTT
TGATCAGATCAATGAAGCGCTGCACGCGACCGAGAAAGGTGAAGCAATCAAACCGGTTCTGGTGT
TTTAA
SEQ ID NO 181: Blakeslea trispora GGPP synthase carG wt (JQ289995.1)
ATGTTGACCTCTAGCAAATCAATTGAATCCTTCCCCAAGAATGTTCAACCTTATGGCAAGCATTAT
CAAAATGGCTTGGAACCTGTTGGAAAAAGCCAAGAAGATATTCTCTTGGAGCCATTCCACTATCTC
TGTTCGAATCCTGGTAAAGATGTCCGAACCAAGATGATTGAAGCGTTCAATGCTTGGCTGAAAGTA
CCCAAGGACGATTTGATCGTCATCACACGTGTGATTGAAATGCTTCATAGTGCTAGTTTGTTAATT
GATGATGTGGAAGATGATTCCGTGTTGCGTCGTGGTGTTCCTGCAGCTCATCATATATATGGTACT
CCTCAAACTATCAATTGTGCTAATTACGTGTACTTTCTTGCACTGAAAGAAATTGCCAAGTTGAAC
AAGCCCAACATGATTACTATCTATACCGATGAATTGATCAATTTGCACAGAGGGCAAGGAATGGA
ATTGTTTTGGCGTGACACCTTAACTTGTCCTACAGAGAAAGAATTTCTTGACATGGTAAACGACAA
AACTGGTGGCCTCTTGAGATTAGCTGTGAAACTTATGCAAGAAGCTAGTCAATCGGGAACTGATTA
TACGGGACTCGTAAGTAAGATTGGTATCCATTTCCAAGTACGCGACGATTATATGAATTTGCAGTC
AAAAAACTATGCTGACAACAAAGGATTCTGCGAAGACTTGACAGAAGGAAAATTCTCTTTCCCTA
TTATACATTCAATCCGCTCTGACCCAAGCAATCGCCAGCTTTTGAACATTTTAAAACAGCGCAGTA
GCTCTATCGAACTCAAGCAATTTGCCTTGCAGCTACTGGAAAACACAAACACTTTCCAATACTGTC
GTGATTTCTTACGTGTCTTGGAAAAGGAAGCTAGAGAAGAAATTAAGCTTTTAGGGGGTAACATC
ATGTTGGAGAAAATTATGGATGTCTTGAGTGTCAATGAATAA
SEQ ID NO 182: Blakeslea trispora GGPP synthase carG wt (AFC92798.1)
MLTSSKSIESFPKNVQPYGKHYQNGLEPVGKSQEDILLEPFHYLCSNPGKDVRTKMIEAFNAWLKVPK
DDLIVITRVIEMLHSASLLIDDVEDDSVLRRGVPAAHHIYGTPQTINCANYVYFLALKEIAKLNKPNMITI
YTDELINLHRGQGMELFWRDTLTCPTEKEFLDMVNDKTGGLLRLAVKLMQEASQSGTDYTGLVSKIGI
HFQVRDDYMNLQSKNYADNKGFCEDLTEGKFSFPIIHSIRSDPSNRQLLNILKQRSSSIELKQFALQLLE
NTNTFQYCRDFLRVLEKEAREEIKLLGGNIMLEKIMDVLSVNE*
SEQ ID NO 183: Blakeslea trispora GGPP synthase carG Yeast optimized
ATGTTGACATCTTCTAAGTCCATCGAATCTTTCCCAAAGAACGTTCAACCATACGGTAAACACTAT
CAAAACGGTTTAGAACCAGTCGGTAAGTCTCAAGAAGACATCTTGTTGGAACCTTTCCACTACTTA
TGTTCTAATCCAGGTAAGGATGTTAGAACCAAGATGATTGAAGCTTTCAACGCCTGGTTGAAAGTC
CCAAAGGACGATTTGATTGTTATCACCAGAGTCATTGAAATGTTGCACTCCGCTTCTTTGTTGATTG
ATGACGTCGAGGACGATTCTGTCTTGAGAAGAGGTGTCCCAGCCGCCCACCATATCTACGGTACCC
CTCAAACCATCAACTGCGCTAACTACGTTTATTTCTTGGCCTTGAAAGAAATCGCCAAGTTGAACA
AGCCAAATATGATTACTATTTATACCGATGAATTGATCAACTTGCACAGAGGTCAAGGTATGGAAT
TGTTCTGGCGTGATACCTTGACCTGCCCAACTGAGAAAGAGTTTTTGGATATGGTTAACGATAAGA
CTGGTGGTTTGTTGAGATTGGCCGTCAAGTTGATGCAAGAGGCTTCTCAATCTGGTACCGACTATA
CTGGTTTGGTTTCTAAGATCGGTATCCATTTTCAAGTTAGAGATGACTACATGAACTTGCAATCCA
AAAACTACGCCGATAATAAGGGTTTCTGTGAAGATTTGACCGAAGGTAAGTTCTCCTTTCCAATTA
TTCACTCTATCAGATCTGACCCATCCAACAGACAATTATTGAATATTTTGAAGCAAAGATCTTCTTC
TATTGAATTGAAACAATTCGCTTTACAATTGTTAGAAAACACTAACACTTTTCAATACTGTAGAGA
TTTCTTGAGAGTTTTGGAAAAGGAAGCCAGAGAAGAGATCAAATTATTGGGTGGTAACATCATGTT
GGAAAAGATTATGGACGTCTTGTCTGTTAATGAATAA
SEQ ID NO 184: Salvia miltiorrhiza SmCPS2 wt (EU003997.1 73-2454 (+))
ATGGCCTCCTTATCCTCTACAATCCTCAGCCGCTCTCCGGCGGCCCGCCGCAGAATTACGCCGGCG
TCGGCTAAGCTTCACCGGCCGGAATGTTTCGCCACCAGTGCATGGATGGGCAGCAGCAGTAAAAA
CCTTTCTCTCAGCTACCAACTTAATCACAAGAAAATATCAGTTGCCACAGTAGATGCGCCGCAGGT
GCATGACCACGACGGCACTACCGTTCATCAAGGCCATGATGCGGTGAAGAATATTGAGGATCCCA
TTGAATACATCAGGACGTTGTTGAGGACGACGGGGGACGGGAGAATAAGCGTGTCGCCGTACGAC
ACGGCGTGGGTGGCGATGATCAAGGACGTGGAGGGGCGGGACGGCCCCCAGTTCCCCTCCAGCCT
CGAGTGGATCGTGCAGAATCAACTCGAGGATGGATCGTGGGGCGATCAGAAGCTTTTCTGCGTCT
ACGATCGCCTCGTCAATACCATCGCGTGCGTGGTAGCCTTGAGATCGTGGAATGTTCATGCTCACA
AGGTCAAAAGAGGAGTGACGTACATCAAGGAAAATGTGGATAAACTTATGGAGGGAAATGAGGA
GCACATGACTTGTGGGTTCGAAGTGGTGTTTCCGGCGCTTCTACAAAAAGCGAAAAGCTTAGGCAT
CGAAGATCTTCCTTACGATTCTCCGGCGGTGCAGGAGGTTTATCATGTCAGGGAACAAAAGTTGAA
AAGGATTCCACTGGAGATTATGCACAAAATACCGACATCATTATTATTTAGTTTGGAAGGGCTCGA
AAATTTGGATTGGGACAAACTTTTGAAACTGCAGTCAGCCGACGGTTCCTTCCTCACCTCTCCCTCC
TCCACCGCCTTCGCGTTCATGCAAACCAAGGATGAAAAATGCTACCAATTCATCAAGAACACGAT
AGACACTTTCAACGGAGGAGCGCCACACACTTATCCCGTCGACGTGTTTGGAAGGCTCTGGGCGAT
CGACCGGCTGCAGCGCCTCGGAATTTCCCGCTTTTTTGAGCCGGAGATTGCTGATTGCTTAAGCCA
CATCCACAAATTTTGGACGGATAAGGGAGTTTTCAGTGGGAGAGAATCGGAGTTTTGCGACATTG
ACGATACATCCATGGGAATGAGGCTTATGAGGATGCATGGATATGATGTTGATCCAAATGTGCTG
AGGAATTTCAAGCAGAAAGATGGTAAATTCTCTTGCTACGGCGGGCAGATGATCGAGTCGCCTTCT
CCGATATACAATCTTTACAGAGCTTCTCAGCTCCGATTTCCCGGCGAGGAAATCCTCGAAGATGCG
AAGAGATTCGCCTACGATTTCTTGAAAGAAAAACTAGCCAACAATCAGATTCTGGATAAATGGGT
TATTTCTAAGCACTTGCCTGATGAGATCAAGCTCGGGCTAGAGATGCCGTGGCTCGCCACCCTACC
CCGCGTCGAGGCGAAGTACTACATCCAGTACTACGCCGGCTCCGGCGACGTGTGGATCGGAAAGA
CGCTGTACAGGATGCCGGAGATCAGCAACGACACGTACCACGACCTAGCCAAGACGGATTTCAAG
AGATGCCAAGCGAAGCATCAGTTCGAGTGGCTCTACATGCAAGAATGGTACGAGAGCTGCGGCAT
CGAGGAATTCGGGATAAGCAGAAAGGACCTTCTGCTTTCCTATTTCTTGGCGACCGCGAGCATCTT
CGAGCTCGAGAGGACCAACGAGCGAATCGCGTGGGCCAAATCGCAGATCATCGCTAAGATGATCA
CTTCTTTCTTCAACAAGGAAACTACGTCGGAGGAGGACAAGCGAGCTCTTTTGAACGAGCTCGGA
AACATTAATGGCCTCAACGACACAAACGGCGCAGGGAGAGAAGGTGGGGCCGGTAGCATTGCGCT
AGCGACCCTCACTCAGTTCCTCGAGGGATTCGACAGATACACCAGACACCAGCTGAAAAATGCTT
GGAGCGTATGGCTGACGCAGCTGCAACATGGCGAAGCAGACGACGCGGAGCTCCTAACCAACACG
TTGAACATCTGCGCCGGCCACATCGCCTTCAGGGAAGAAATACTGGCGCACAACGAGTACAAAGC
TCTCTCCAACCTAACCAGCAAAATCTGTCGACAGCTTTCTTTCATTCAAAGCGAAAAGGAGATGGG
AGTAGAGGGCGAGATCGCAGCGAAATCGAGCATAAAAAACAAGGAACTCGAAGAAGACATGCAA
ATGTTGGTGAAGTTGGTGCTTGAGAAATATGGGGGCATAGATAGAAATATAAAGAAAGCGTTTTT
AGCAGTTGCGAAGACTTATTATTACAGAGCGTATCATGCCGCCGACACCATAGACACACACATGTT
TAAAGTGCTTTTCGAGCCAGTCGCGTGA
SEQ ID NO 185: Salvia miltiorrhiza SmCPS2
MATVDAPQVHDHDGTTVHQGHDAVKNIEDPIEYIRTLLRTTGDGRISVSPYDTAWVAMIKDVEGRDG
PQFPSSLEWIVQNQLEDGSWGDQKLFCVYDRLVNTIACVVALRSWNVHAHKVKRGVTYIKENVDKL
MEGNEEHMTCGFEVVFPALLQKAKSLGIEDLPYDSPAVQEVYHVREQKLKRIPLEIMHKIPTSLLFSLEG
LENLDWDKLLKLQSADGSFLTSPSSTAFAFMQTKDEKCYQFIKNTIDTFNGGAPHTYPVDVFGRLWAI
DRLQRLGISRFFEPEIADCLSHIHKFWTDKGVFSGRESEFCDIDDTSMGMRLMRMHGYDVDPNVLRNF
KQKDGKFSCYGGQMIESPSPIYNLYRASQLRFPGEEILEDAKRFAYDFLKEKLANNQILDKWVISKHLP
DEIKLGLEMPWLATLPRVEAKYYIQYYAGSGDVWIGKTLYRMPEISNDTYHDLAKTDFKRCQAKHQF
EWLYMQEWYESCGIEEFGISRKDLLLSYFLATASIFELERTNERIAWAKSQIIAKMITSFFNKETTSEEDK
RALLNELGNINGLNDTNGAGREGGAGSIALATLTQFLEGFDRYTRHQLKNAWSVWLTQLQHGEADDA
ELLTNTLNICAGHIAFREEILAHNEYKALSNLTSKICRQLSFIQSEKEMGVEGEIAAKSSIKNKELEEDMQ
MLVKLVLEKYGGIDRNIKKAFLAVAKTYYYRAYHAADTIDTHMFKVLFEPVA*
SEQ ID NO 186: Salvia miltiorrhiza SmCPS2 Yeast optimized
ATGGCTACTGTTGACGCTCCACAAGTTCACGACCACGACGGTACTACTGTTCACCAAGGTCACGAC
GCTGTTAAGAACATCGAAGACCCAATCGAATACATCAGAACTTTGTTGAGAACTACTGGTGACGG
TAGAATCTCTGTTTCTCCATACGACACTGCTTGGGTTGCTATGATCAAGGACGTTGAAGGTAGAGA
CGGTCCACAATTCCCATCTTCTTTGGAATGGATCGTTCAAAACCAATTGGAAGACGGTTCTTGGGG
TGACCAAAAGTTGTTCTGTGTTTACGACAGATTGGTTAACACTATCGCTTGTGTTGTTGCTTTGAGA
TCTTGGAACGTTCACGCTCACAAGGTTAAGAGAGGTGTTACTTACATCAAGGAAAACGTTGACAA
GTTGATGGAAGGTAACGAAGAACACATGACTTGTGGTTTCGAAGTTGTTTTCCCAGCTTTGTTGCA
AAAGGCTAAGTCTTTGGGTATCGAAGACTTGCCATACGACTCTCCAGCTGTTCAAGAAGTTTACCA
CGTTAGAGAACAAAAGTTGAAGAGAATCCCATTGGAAATCATGCACAAGATCCCAACTTCTTTGTT
GTTCTCTTTGGAAGGTTTGGAAAACTTGGACTGGGACAAGTTGTTGAAGTTGCAATCTGCTGACGG
TTCTTTCTTGACTTCTCCATCTTCTACTGCTTTCGCTTTCATGCAAACTAAGGACGAAAAGTGTTAC
CAATTCATCAAGAACACTATCGACACTTTCAACGGTGGTGCTCCACACACTTACCCAGTTGACGTT
TTCGGTAGATTGTGGGCTATCGACAGATTGCAAAGATTGGGTATCTCTAGATTCTTCGAACCAGAA
ATCGCTGACTGTTTGTCTCACATCCACAAGTTCTGGACTGACAAGGGTGTTTTCTCTGGTAGAGAA
TCTGAATTCTGTGACATCGACGACACTTCTATGGGTATGAGATTGATGAGAATGCACGGTTACGAC
GTTGACCCAAACGTTTTGAGAAACTTCAAGCAAAAGGACGGTAAGTTCTCTTGTTACGGTGGTCAA
ATGATCGAATCTCCATCTCCAATCTACAACTTGTACAGAGCTTCTCAATTGAGATTCCCAGGTGAA
GAAATCTTGGAAGACGCTAAGAGATTCGCTTACGACTTCTTGAAGGAAAAGTTGGCTAACAACCA
AATCTTGGACAAGTGGGTTATCTCTAAGCACTTGCCAGACGAAATCAAGTTGGGTTTGGAAATGCC
ATGGTTGGCTACTTTGCCAAGAGTTGAAGCTAAGTACTACATCCAATACTACGCTGGTTCTGGTGA
CGTTTGGATCGGTAAGACTTTGTACAGAATGCCAGAAATCTCTAACGACACTTACCACGACTTGGC
TAAGACTGACTTCAAGAGATGTCAAGCTAAGCACCAATTCGAATGGTTGTACATGCAAGAATGGT
ACGAATCTTGTGGTATCGAAGAATTCGGTATCTCTAGAAAGGACTTGTTGTTGTCTTACTTCTTGGC
TACTGCTTCTATCTTCGAATTGGAAAGAACTAACGAAAGAATCGCTTGGGCTAAGTCTCAAATCAT
CGCTAAGATGATCACTTCTTTCTTCAACAAGGAAACTACTTCTGAAGAAGACAAGAGAGCTTTGTT
GAACGAATTGGGTAACATCAACGGTTTGAACGACACTAACGGTGCTGGTAGAGAAGGTGGTGCTG
GTTCTATCGCTTTGGCTACTTTGACTCAATTCTTGGAAGGTTTCGACAGATACACTAGACACCAATT
GAAGAACGCTTGGTCTGTTTGGTTGACTCAATTGCAACACGGTGAAGCTGACGACGCTGAATTGTT
GACTAACACTTTGAACATCTGTGCTGGTCACATCGCTTTCAGAGAAGAAATCTTGGCTCACAACGA
ATACAAGGCTTTGTCTAACTTGACTTCTAAGATCTGTAGACAATTGTCTTTCATCCAATCTGAAAAG
GAAATGGGTGTTGAAGGTGAAATCGCTGCTAAGTCTTCTATCAAGAACAAGGAATTGGAAGAAGA
CATGCAAATGTTGGTTAAGTTGGTTTTGGAAAAGTACGGTGGTATCGACAGAAACATCAAGAAGG
CTTTCTTGGCTGTTGCTAAGACTTACTACTACAGAGCTTACCACGCTGCTGACACTATCGACACTCA
CATGTTCAAGGTTTTGTTCGAACCAGTTGCTTAA
SEQ ID NO 187: Salvia sclarea SsLPS wt (JN133923.1)
ATGACTTCTGTAAATTTGAGCAGAGCACCAGCAGCGATTACCCGGCGCAGGCTGCAGCTACAGCC
GGAATTTCATGCCGAGTGTTCATGGCTGAAAAGCAGCAGCAAACACGCGCCCTTGACCTTGAGTTG
CCAAATCCGTCCTAAGCAACTCTCCCAAATAGCTGAATTGAGAGTAACAAGCCTGGATGCGTCGC
AAGCGAGTGAAAAAGACATTTCCCTTGTTCAAACTCCGCATAAGGTTGAGGTTAATGAAAAGATC
GAGGAGTCAATCGAGTACGTCCAAAATCTGTTGATGACGTCGGGCGACGGGCGAATAAGCGTGTC
ACCCTATGACACGGCAGTGATCGCCCTGATCAAGGACTTGAAAGGGCGCGACGCCCCGCAGTTTC
CGTCATGTCTCGAGTGGATCGCGCACCACCAACTGGCTGATGGCTCATGGGGCGACGAATTCTTCT
GTATTTATGATCGGATTCTAAATACATTGGCATGTGTCGTAGCCTTGAAATCATGGAACCTTCACTC
TGATATTATTGAAAAAGGAGTGACGTACATCAAGGAGAATGTGCATAAACTTAAAGGTGCAAATG
TTGAGCACAGGACAGCGGGGTTCGAACTTGTGGTTCCTACTTTTATGCAAATGGCCACAGATTTGG
GCATCCAAGATCTGCCCTATGATCATCCCCTCATCAAGGAGATTGCTGACACAAAACAACAAAGA
TTGAAAGAGATACCCAAGGATTTGGTTTACCAAATGCCAACGAATTTACTGTACAGTTTAGAAGGG
TTAGGAGATTTGGAGTGGGAAAGGCTACTGAAACTGCAGTCGGGCAATGGCTCCTTCCTCACTTCG
CCGTCGTCCACCGCCGCCGTCTTGATGCATACCAAAGATGAAAAATGTTTGAAATACATCGAAAAC
GCCCTCAAGAATTGCGACGGAGGAGCACCACATACTTATCCAGTCGATATCTTCTCAAGACTTTGG
GCAATCGATAGGCTACAACGCCTAGGAATTTCTCGTTTCTTCCAGCACGAGATCAAGTATTTCTTA
GATCACATCGAAAGCGTTTGGGAGGAGACCGGAGTTTTCAGTGGAAGATATACGAAATTTAGCGA
TATTGATGACACGTCCATGGGCGTTAGGCTTCTCAAAATGCACGGATACGACGTCGATCCAAATGT
ACTAAAACATTTCAAGCAACAAGATGGTAAATTTTCCTGCTACATTGGTCAATCGGTCGAGTCTGC
ATCTCCAATGTACAATCTTTATAGGGCTGCTCAACTAAGATTTCCAGGAGAAGAAGTTCTTGAAGA
AGCCACTAAATTTGCCTTTAACTTCTTGCAAGAAATGCTAGTCAAAGATCGACTTCAAGAAAGATG
GGTGATATCCGACCACTTATTTGATGAGATAAAGCTGGGGTTGAAGATGCCATGGTACGCCACTCT
ACCCCGAGTCGAGGCTGCATATTATCTAGACCATTATGCTGGTTCTGGTGATGTATGGATTGGCAA
GAGTTTCTACAGGATGCCAGAAATCAGCAATGATACATACAAGGAGCTTGCGATATTGGATTTCA
ACAGATGCCAAACACAACATCAGTTGGAGTGGATCCACATGCAGGAATGGTACGACAGATGCAGC
CTTAGCGAATTCGGGATAAGCAAAAGAGAGTTGCTTCGCTCTTACTTTCTGGCCGCAGCAACCATA
TTCGAACCGGAGAGAACTCAAGAGAGGCTTCTGTGGGCCAAAACCAGAATTCTTTCTAAGATGAT
CACTTCATTTGTCAACATTAGTGGAACAACACTATCTTTGGACTACAATTTCAATGGCCTCGATGA
AATAATTAGTAGTGCCAATGAAGATCAAGGACTGGCTGGGACTCTGCTGGCAACCTTCCATCAACT
TCTAGACGGATTCGATATATACACTCTCCATCAACTCAAACATGTTTGGAGCCAATGGTTCATGAA
AGTGCAGCAAGGAGAGGGAAGCGGCGGGGAAGACGCGGTGCTCCTAGCGAACACGCTCAACATC
TGCGCCGGCCTCAACGAAGACGTGTTGTCCAACAATGAATACACGGCTCTGTCCACCCTCACAAAT
AAAATCTGCAATCGCCTCGCCCAAATTCAAGACAATAAGATTCTCCAAGTTGTGGATGGGAGCAT
AAAGGATAAGGAGCTAGAACAGGATATGCAGGCGTTGGTGAAGTTAGTGCTTCAAGAAAATGGCG
GCGCCGTAGACAGAAACATCAGACACACGTTTTTGTCGGTTTCCAAGACTTTCTACTACGATGCCT
ACCACGACGATGAGACGACCGATCTTCATATCTTCAAAGTACTCTTTCGACCGGTTGTATGA
SEQ ID NO 188: Salvia sclarea SsLPS E. coli optimized
MASQASEKDISLVQTPHKVEVNEKIEESIEYVQNLLMTSGDGRISVSPYDTAVIALIKDLKGRDAPQFPS
CLEWIAHHQLADGSWGDEFFCIYDRILNTLACVVALKSWNLHSDIIEKGVTYIKENVHKLKGANVEHR
TAGFELVVPTFMQMATDLGIQDLPYDHPLIKEIADTKQQRLKEIPKDLVYQMPTNLLYSLEGLGDLEW
ERLLKLQSGNGSFLTSPSSTAAVLMHTKDEKCLKYIENALKNCDGGAPHTYPVDIFSRLWAIDRLQRLG
ISRFFQHEIKYFLDHIESVWEETGVFSGRYTKFSDIDDTSMGVRLLKMHGYDVDPNVLKHFKQQDGKFS
CYIGQSVESASPMYNLYRAAQLRFPGEEVLEEATKFAFNFLQEMLVKDRLQERWVISDHLFDEIKLGLK
MPWYATLPRVEAAYYLDHYAGSGDVWIGKSFYRMPEISNDTYKELAILDFNRCQTQHQLEWIHMQE
WYDRCSLSEFGISKRELLRSYFLAAATIFEPERTQERLLWAKTRILSKMITSFVNISGTTLSLDYNFNGLD
EIISSANEDQGLAGTLLATFHQLLDGFDIYTLHQLKHVWSQWFMKVQQGEGSGGEDAVLLANTLNICA
GLNEDVLSNNEYTALSTLTNKICNRLAQIQDNKILQVVDGSIKDKELEQDMQALVKLVLQENGGAVDR
NIRHTFLSVSKTFYYDAYHDDETTDLHIFKVLFRPVV*
SEQ ID NO 189: Salvia sclarea SsLPS E. coli optimized
ATGGCATCCCAAGCGTCCGAGAAAGATATTAGCCTGGTTCAAACCCCGCATAAGGTCGAGGTCAA
CGAAAAGATCGAAGAGAGCATCGAGTACGTCCAAAATCTGCTGATGACGAGCGGTGACGGTCGTA
TCTCCGTGTCTCCGTACGATACCGCGGTCATCGCTCTGATTAAAGATCTGAAGGGTCGCGACGCAC
CGCAGTTCCCGAGCTGTCTGGAGTGGATTGCGCACCACCAGTTAGCGGATGGTAGCTGGGGCGAC
GAGTTCTTTTGTATCTATGACCGCATTTTGAATACCCTGGCGTGCGTCGTCGCACTGAAATCTTGGA
ATCTGCACAGCGACATTATTGAAAAAGGCGTGACCTACATTAAGGAAAACGTCCATAAGCTGAAA
GGCGCGAATGTTGAGCATAGAACCGCCGGTTTTGAGCTGGTTGTTCCGACCTTCATGCAGATGGCG
ACTGACCTGGGTATTCAGGATCTGCCGTACGATCATCCTCTTATCAAAGAAATCGCTGATACGAAG
CAACAGCGCCTGAAAGAAATTCCGAAAGATTTGGTTTATCAGATGCCGACCAATCTGCTGTATAGC
CTGGAAGGCCTGGGCGATTTAGAGTGGGAGCGTTTGCTGAAGCTGCAGTCTGGTAATGGTAGCTTC
CTGACGAGCCCAAGCAGCACGGCGGCAGTTCTGATGCATACCAAAGACGAGAAGTGTTTGAAATA
CATTGAGAATGCGCTGAAGAACTGCGACGGTGGCGCTCCTCATACGTATCCGGTTGACATCTTTAG
CCGCTTGTGGGCGATCGACCGTTTGCAACGTCTGGGCATTAGCCGTTTCTTCCAACACGAGATCAA
ATACTTTCTGGACCACATCGAGTCAGTCTGGGAAGAAACCGGCGTGTTTAGCGGTCGTTACACGAA
GTTTAGCGACATCGATGACACGAGCATGGGTGTCCGCCTGCTGAAAATGCACGGTTACGACGTAG
ACCCAAACGTGTTGAAACACTTTAAGCAGCAAGACGGCAAATTCAGCTGCTACATCGGCCAGAGC
GTCGAGAGCGCGAGCCCGATGTATAATCTGTACCGTGCCGCCCAGCTGCGTTTCCCGGGTGAAGA
AGTGCTTGAAGAAGCAACTAAATTCGCGTTTAACTTCCTGCAAGAGATGCTGGTGAAGGATCGCTT
GCAAGAGCGTTGGGTTATTAGCGATCACCTGTTTGACGAGATTAAGCTCGGTCTGAAGATGCCGTG
GTATGCTACCCTGCCGCGTGTTGAGGCCGCTTATTACCTGGATCACTATGCGGGTAGCGGTGATGT
GTGGATTGGTAAGTCTTTTTACCGCATGCCGGAGATTAGCAATGACACCTACAAAGAATTGGCCAT
CCTGGACTTTAACCGTTGTCAGACTCAGCATCAGCTGGAGTGGATTCACATGCAAGAGTGGTATGA
CCGCTGCTCTCTGTCCGAGTTTGGTATTAGCAAGCGTGAGCTGCTGCGTAGCTACTTCCTGGCTGCC
GCAACCATTTTCGAACCGGAACGCACCCAAGAGCGTCTGCTCTGGGCAAAGACCCGCATCCTGAG
CAAGATGATTACCAGCTTCGTCAACATCTCCGGTACGACCCTGAGCCTGGATTACAACTTCAACGG
TTTGGATGAGATCATTTCCAGCGCGAATGAAGATCAGGGTCTGGCGGGTACGCTGTTGGCCACGTT
CCATCAACTGCTGGATGGTTTCGACATTTACACCCTGCACCAACTGAAACACGTCTGGTCGCAATG
GTTTATGAAAGTTCAGCAAGGCGAGGGCTCCGGCGGCGAAGATGCGGTCCTGCTGGCAAATACTC
TGAATATCTGCGCGGGTCTGAATGAAGATGTGCTGTCGAACAACGAGTATACCGCGCTGAGCACG
CTGACGAACAAGATCTGCAACCGTCTGGCCCAGATCCAGGACAACAAGATTCTGCAAGTGGTGGA
CGGCAGCATCAAAGACAAAGAACTGGAACAGGATATGCAGGCATTGGTTAAACTGGTGCTGCAGG
AAAACGGTGGCGCAGTGGACCGTAACATCCGTCACACGTTTCTGAGCGTTAGCAAGACCTTCTACT
ATGACGCGTATCACGACGATGAAACCACCGATCTGCATATCTTTAAAGTCCTGTTCCGTCCGGTTG
TTTAA
SEQ ID NO 190: Pantoea asslomerans CrtE wt (M38424.1 40-963 (+))
ATGGTGAGTGGCAGTAAAGCGGGCGTTTCGCCTCATCGCGAAATAGAAGTAATGAGACAATCCAT
TGACGATCACCTGGCTGGCCTGTTACCTGAAACCGACAGCCAGGATATCGTCAGCCTTGCGATGCG
TGAAGGCGTCATGGCACCCGGTAAACGGATCCGTCCGCTGCTGATGCTGCTGGCCGCCCGCGACCT
CCGCTACCAGGGCAGTATGCCTACGCTGCTCGATCTCGCCTGCGCCGTTGAACTGACCCATACCGC
GTCGCTGATGCTCGACGACATGCCCTGCATGGACAACGCCGAGCTGCGCCGCGGTCAGCCCACTA
CCCACAAAAAATTTGGTGAGAGCGTGGCGATCCTTGCCTCCGTTGGGCTGCTCTCTAAAGCCTTTG
GTCTGATCGCCGCCACCGGCGATCTGCCGGGGGAGAGGCGTGCCCAGGCGGTCAACGAGCTCTCT
ACCGCCGTGGGCGTGCAGGGCCTGGTACTGGGGCAGTTTCGCGATCTTAACGATGCCGCCCTCGAC
CGTACCCCTGACGCTATCCTCAGCACCAACCACCTCAAGACCGGCATTCTGTTCAGCGCGATGCTG
CAGATCGTCGCCATTGCTTCCGCCTCGTCGCCGAGCACGCGAGAGACGCTGCACGCCTTCGCCCTC
GACTTCGGCCAGGCGTTTCAACTGCTGGACGATCTGCGTGACGATCACCCGGAAACCGGTAAAGA
TCGCAATAAGGACGCGGGAAAATCGACGCTGGTCAACCGGCTGGGCGCAGACGCGGCCCGGCAA
AAGCTGCGCGAGCATATTGATTCCGCCGACAAACACCTCACTTTTGCCTGTCCGCAGGGCGGCGCC
ATCCGACAGTTTATGCATCTGTGGTTTGGCCATCACCTTGCCGACTGGTCACCGGTCATGAAAATC
GCCTGA
SEQ ID NO 191: Pantoea agglomerans CrtE wt (AAA24819.1)
MVSGSKAGVSPHREIEVMRQSIDDHLAGLLPETDSQDIVSLAMREGVMAPGKRIRPLLMLLAARDLRY
QGSMPTLLDLACAVELTHTASLMLDDMPCMDNAELRRGQPTTHKKFGESVAILASVGLLSKAFGLIAA
TGDLPGERRAQAVNELSTAVGVQGLVLGQFRDLNDAALDRTPDAILSTNHLKTGILFSAMLQIVAIASA
SSPSTRETLHAFALDFGQAFQLLDDLRDDHPETGKDRNKDAGKSTLVNRLGADAARQKLREHIDSADK
HLTFACPQGGAIRQFMHLWFGHHLADWSPVMKIA*
SEQ ID NO 192: Pantoea asslomerans CrtE Yeast optimized
ATGGTTTCTGGTTCGAAAGCAGGAGTATCACCTCATAGGGAAATCGAAGTCATGAGACAGTCCATT
GATGACCACTTAGCAGGATTGTTGCCAGAAACAGATTCCCAGGATATCGTTAGCCTTGCTATGAGA
GAAGGTGTTATGGCACCTGGTAAACGTATCAGACCTTTGCTGATGTTACTTGCTGCAAGAGACCTG
AGATATCAGGGTTCTATGCCTACACTACTGGATCTAGCTTGTGCTGTTGAACTGACACATACTGCTT
CCTTGATGCTGGATGACATGCCTTGTATGGACAATGCGGAACTTAGAAGAGGTCAACCAACAACC
CACAAGAAATTCGGAGAATCTGTTGCCATTTTGGCTTCTGTAGGTCTGTTGTCGAAAGCATTTGGC
TTGATTGCTGCAACTGGTGATCTTCCAGGTGAAAGGAGAGCACAAGCTGTAAACGAGCTATCTACT
GCAGTTGGTGTTCAAGGTCTAGTCTTAGGACAGTTCAGAGATTTGAATGACGCAGCTTTGGACAGA
ACTCCTGATGCTATCCTGTCTACGAACCATCTGAAGACTGGCATCTTGTTCTCAGCTATGTTGCAAA
TCGTAGCCATTGCTTCTGCTTCTTCACCATCTACTAGGGAAACGTTACACGCATTCGCATTGGACTT
TGGTCAAGCCTTTCAACTGCTAGACGATTTGAGGGATGATCATCCAGAGACAGGTAAAGACCGTA
ACAAAGACGCTGGTAAAAGCACTCTAGTCAACAGATTGGGTGCTGATGCAGCTAGACAGAAACTG
AGAGAGCACATTGACTCTGCTGACAAACACCTGACATTTGCATGTCCACAAGGAGGTGCTATAAG
GCAGTTTATGCACCTATGGTTTGGACACCATCTTGCTGATTGGTCTCCAGTGATGAAGATCGCCTA
A
SEQ ID NO 193: Talaromyces verruculosus TalVeTPP wt (LHCL01000010.1
150095-151030 (+))
ATGTCTAATGACACCACTACCACGGCTTCTGCCGGAACAGCAACTTCTTCGCGGTTTCTTTCCGTGG
GGGGAGTTGTGAACTTCCGTGAACTGGGCGGTTACCCATGTGATTCTGTCCCTCCTGCTCCTGCCTC
AAACGGCTCACCGGACAATGCATCTGAAGCGACCCTTTGGGTTGGCCACTCGTCCATTCGGCCTGG
ATTTCTGTTTCGATCGGCACAGCCGTCTCAGATTACCCCGGCCGGTATTGAGACATTGATCCGCCA
GCTTGGCATCCAGACAATTTTTGACTTTCGTTCAAGGACGGAAATTGAGCTTGTTGCCACTCGCTAT
CCTGATTCGCTACTTGAGATACCTGGCACGACTCGCTATTCCGTGCCCGTCTTCTCGGAAGGCGAC
TATTCCCCAGCGTCATTAGTCAAGAGGTACGGAGTGTCCTCCGATACTGCAACCGATTCCACTTCC
TCCAAAAGTGCTAAGCCTACAGGATTCGTCCACGCATATGAGGCTATCGCACGCAGTGCAGCAGA
AAACGGCAGTTTTCGTAAGATAACGGACCACATAATACAACATCCGGACCGGCCTATTCTGTTTCA
CTGTACACTGGGGAAAGACCGAACCGGTGTGTTTGCAGCATTGTTATTGAGTCTTTGCGGGGTACC
AGACGAGACGATAGTTGAAGACTATGCTATGACTACCGAGGGATTTGGAGCCTGGCGGGAACATC
TAATTCAACGCTTGCTACAAAGGAAGGATGCAGCTACGCGCGAGGATGCAGAATCCATTATTGCC
AGCCCCCCGGAGACTATGAAGGCTTTTCTAGAAGATGTGGTAGCAGCCAAGTTCGGGGGTGCTCG
AAATTACTTTATCCAGCACTGTGGATTTACGGAAGCTGAGGTTGATAAGTTAAGCCATACACTGGC
CATTACGAATTGA
SEQ ID NO 194: Talaromyces verruculosus TalVeTPP wt (KUL89334.1)
MSNDTTTTASAGTATSSRFLSVGGVVNFRELGGYPCDSVPPAPASNGSPDNASEATLWVGHSSIRPGFL
FRSAQPSQITPAGIETLIRQLGIQTIFDFRSRTEIELVATRYPDSLLEIPGTTRYSVPVFSEGDYSPASLVKR
YGVSSDTATDSTSSKSAKPTGFVHAYEAIARSAAENGSFRKITDHIIQHPDRPILFHCTLGKDRTGVFAA
LLLSLCGVPDETIVEDYAMTTEGFGAWREHLIQRLLQRKDAATREDAESIIASPPETMKAFLEDVVAAK
FGGARNYFIQHCGFTEAEVDKLSHTLAITN
SEQ ID NO 195: Talaromyces verruculosus TalVeTPP Yeast optimized
ATGTCTAACGACACTACTACTACTGCTTCTGCTGGTACTGCTACTTCTTCTAGATTCTTGTCTGTTG
GTGGTGTTGTTAACTTCAGAGAATTGGGTGGTTACCCATGTGACTCTGTTCCACCAGCTCCAGCTTC
TAACGGTTCTCCAGACAACGCTTCTGAAGCTACTTTGTGGGTTGGTCACTCTTCTATCAGACCAGGT
TTCTTGTTCAGATCTGCTCAACCATCTCAAATCACTCCAGCTGGTATCGAAACTTTGATCAGACAAT
TGGGTATCCAAACTATCTTCGACTTCAGATCTAGAACTGAAATCGAATTGGTTGCTACTAGATACC
CAGACTCTTTGTTGGAAATCCCAGGTACTACTAGATACTCTGTTCCAGTTTTCTCTGAAGGTGACTA
CTCTCCAGCTTCTTTGGTTAAGAGATACGGTGTTTCTTCTGACACTGCTACTGACTCTACTTCTTCTA
AGTCTGCTAAGCCAACTGGTTTCGTTCACGCTTACGAAGCTATCGCTAGATCTGCTGCTGAAAACG
GTTCTTTCAGAAAGATCACTGACCACATCATCCAACACCCAGACAGACCAATCTTGTTCCACTGTA
CTTTGGGTAAGGACAGAACTGGTGTTTTCGCTGCTTTGTTGTTGTCTTTGTGTGGTGTTCCAGACGA
AACTATCGTTGAAGACTACGCTATGACTACTGAAGGTTTCGGTGCTTGGAGAGAACACTTGATCCA
AAGATTGTTGCAAAGAAAGGACGCTGCTACTAGAGAAGACGCTGAATCTATCATCGCTTCTCCACC
AGAAACTATGAAGGCTTTCTTGGAAGACGTTGTTGCTGCTAAGTTCGGTGGTGCTAGAAACTACTT
CATCCAACACTGTGGTTTCACTGAAGCTGAAGTTGACAAGTTGTCTCACACTTTGGCTATCACTAA
CTAA
SEQ ID NO 196: Artificial RBS sequence
AAGGAGGTAAAAAA
SEQ ID NO 197: Artificial BYMO sequence motif 8
GAGxSGL

X₄ can be any naturally occurring amino acid, particularly A or I

The numbering of X corresponds to its position in the sequence.

SEQ ID NO 198:
Artificial BYMO sequence motif 1
EKNxxxxGTWxENRYPGCACDVPxHxYxxSFE

X₄ can be any naturally occurring amino acid, particularly H or P.

X₅ can be any naturally occurring amino acid, particularly A, D, or E.

X₆ can be any naturally occurring amino acid, particularly L or V.

X₇ can be any naturally occurring amino acid, particularly G or S.

X₁₁ can be any naturally occurring amino acid, particularly F, L, or Y.

X₂₄ can be any naturally occurring amino acid, particularly A or S.

X₂₆ can be any naturally occurring amino acid, particularly A, C, or N.

X₂₈ can be any naturally occurring amino acid, particularly A or T.

X₂₉ can be any naturally occurring amino acid, particularly W or Y.

The numbering of X corresponds to its position in the sequence.

SEQ ID NO 199:
Artificial BVMO sequence motif 2
LxNAxGILNxWxxPxIPG

X₂ can be any naturally occurring amino acid, particularly I, L, or V.

X₅ can be any naturally occurring amino acid, particularly G, S, or T.

X₁₀ can be any naturally occurring amino acid, particularly A or Q.

X₁₂ can be any naturally occurring amino acid, particularly K or R.

X₁₃ can be any naturally occurring amino acid, particularly W or Y.

X₁₅ can be any naturally occurring amino acid, particularly G, P, or S.

The numbering of X corresponds to its position in the sequence.

SEQ ID NO 200:
Artificial BVMO sequence motif 3
LxxKxVxxIGxGSSGIQIxPxI

X₂ can be any naturally occurring amino acid, particularly E, K, or N.

X₃ can be any naturally occurring amino acid, particularly D or G.

X₅ can be any naturally occurring amino acid, particularly K, T, or V.

X₇ can be any naturally occurring amino acid, particularly A or G.

X₈ can be any naturally occurring amino acid, particularly L or V.

X₁₁ can be any naturally occurring amino acid, particularly N or S.

X₁₉ can be any naturally occurring amino acid, particularly L or V.

X₂₁ can be any naturally occurring amino acid, particularly A or N.

The numbering of X corresponds to its position in the sequence.

SEQ ID NO 201:
Artificial BVMO sequence motif 4
GCRRxTPGxxYLExL

X₅ can be any naturally occurring amino acid, particularly L or P.

X₉ can be any naturally occurring amino acid, particularly P or T.

X₁₀ can be any naturally occurring amino acid, particularly G, H, or N.

X₁₄ can be any naturally occurring amino acid, particularly A or S.

The numbering of X corresponds to its position in the sequence.

SEQ ID NO 202:
Artificial BVMO sequence motif 5
CATGFDxxxxPRFxxxG

X₇ can be any naturally occurring amino acid, particularly T or V.

X₈ can be any naturally occurring amino acid, particularly S or T.

X₉ can be any naturally occurring amino acid, particularly F or Y.

X₁₀ can be any naturally occurring amino acid, particularly K or R.

X₁₄ can be any naturally occurring amino acid, particularly K or P.

X₁₅ can be any naturally occurring amino acid, particularly F or L.

X₁₆ can be any naturally occurring amino acid, particularly I or V.

The numbering of X corresponds to its position in the sequence.

SEQ ID NO 203:
Artificial BVMO sequence motif 6
PNxFxxxGPNxPxxNGxV

X₃ can be any naturally occurring amino acid, particularly S or Y.

X₅ can be any naturally occurring amino acid, particularly F, I, or S.

X₆ can be any naturally occurring amino acid, particularly F, I, or T.

X₇ can be any naturally occurring amino acid, particularly L or M.

X₁₁ can be any naturally occurring amino acid, particularly C or G.

X₁₃ can be any naturally occurring amino acid, particularly I or V.

X₁₄ can be any naturally occurring amino acid, particularly A or G.

X₁₇ can be any naturally occurring amino acid, particularly P or S.

The numbering of X corresponds to its position in the sequence.

SEQ ID NO 204:
Artificial BVMO sequence motif 7
AxWPGSxLHYxEAxxxPRxED

X₂ can be any naturally occurring amino acid, particularly L or V.

X₇ can be any naturally occurring amino acid, particularly A or T.

X₁₁ can be any naturally occurring amino acid, particularly L or M.

X₁₄ can be any naturally occurring amino acid, particularly I or L.

X₁₅ can be any naturally occurring amino acid, particularly A, K, or Q.

X₁₆ can be any naturally occurring amino acid, particularly D, H, or S.

X₁₉ can be any naturally occurring amino acid, particularly W or Y.

The numbering of X corresponds to its position in the sequence.

SEQ ID NO 205:
Artificial enal-cleaving polypeptide
sequence motif 1
G-[Y or-]-x-W-x-G-x-x-[F, L or I]-
x-[T, S or R]-G-[H or D]
GxxWxGxxxxxGx

X₂ can be Y or can be deleted.

X₃ can be any naturally occurring amino acid.

X₅ can be any naturally occurring amino acid.

X₇ can be any naturally occurring amino acid.

X₈ can be any naturally occurring amino acid.

X₉ can be F, L, or I.

X₁₀ can be any naturally occurring amino acid.

X₁₁ can be R, S, or T.

X₁₃ can be H or D.

The numbering of X corresponds to its position in the sequence.

SEQ ID NO 206:
Artificial enal-cleaving polypeptide
sequence motif 2
W-[Y, A or V]-G-K-x-[F or Y]-x-[S or D]
WxGKxxxx

X₂ can be A, V, or Y.

X₅ can be any naturally occurring amino acid.

X₆ can be F or Y.

X₇ can be any naturally occurring amino acid.

X₈ can be D or S.

The numbering of X corresponds to its position in the sequence.

SEQ ID NO 207: Artificial enal-
cleaving polypeptide sequence motif 3
[G or S]-x-[A or G]-x-[L or V]-x-x-x-x-
[F, Y or L]-R-G-x-VxxxxxxxxxxRGxV

X₁ can be G or S.

X₂ can be any naturally occurring amino acid.

X₃ can be A or G.

X₄ can be any naturally occurring amino acid.

X₅ can be L or V.

X₆ can be any naturally occurring amino acid.

X₇ can be any naturally occurring amino acid.

X₈ can be any naturally occurring amino acid.

X₉ can be any naturally occurring amino acid.

X₁₀ can be F, L, or Y.

X₁₃ can be any naturally occurring amino acid.

The numbering of X corresponds to its position in the sequence.

SEQ ID NO 208: Artificial enal-cleaving
polypeptide sequence motif 4
[M or L]-[V or I]-Y-D-x-x-P-[I or V]-
x-D-[H or S]-[F or L]xxYDxxPxxDxx

X₁ can be L or M.

X₂ can be I or V.

X₅ can be any naturally occurring amino acid.

X₆ can be any naturally occurring amino acid.

X₈ can be I or V.

X₉ can be any naturally occurring amino acid.

X₁₁ can be H or S.

X₁₂ can be F or L.

The numbering of X corresponds to its position in the sequence.

What is claimed is:

1. A biocatalytic method for preparing an ester compound, comprising:
(1) contacting a carbonyl precursor compound of general formula I 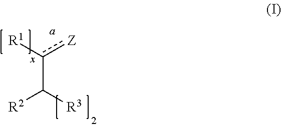
wherein
“a” denotes a single or double bond,
“x” is 1 if “a” denotes a double bond, or “x” is 2 if “a” denotes a single bond
R¹ represent independently of each other H or lower alkyl,
R² represents H, a linear or branched, saturated or unsaturated, optionally substituted hydrocarbyl residue, or a group Cyc-A-,
wherein
Cyc represents an optionally substituted, saturated or unsaturated, mono- or polycyclic hydrocarbyl residue, and
A represents a chemical bond or an optionally substituted, straight chain or branched alkylene bridge,
R³ represent independently of each other H or a C₁-Cis hydrocarbyl group, or a lower alkyl group,
and
when “a” denotes a single bond, then Z represents a hydrocarbyl residue containing a carbonyl group,
and
when “a” denotes a double bond, then Z forms, together with the carbon atom which it is attached to, either a carbonyl group, or an alkylidene residue carrying a terminal carbonyl group
or
when “a” denotes a double bond, and Z forms, together with the carbon atom which it is attached to, a carbonyl group, then R² and R¹, together with the carbon atoms which they are attached to, may also form a cyclic, saturated or unsaturated, optionally substituted carbocyclic ring group,
and wherein said carbonyl compound of general formula I is provided in stereoisomerically pure form, or as a mixture of stereoisomers;
with a polypeptide having Baeyer-Villiger monooxygenase (BVMO) (EC 1.13.14.-) activity, so as to form the respective carbonyl ester,
(2) and optionally isolating the carbonyl ester formed in step (1), wherein said carbonyl ester compound is obtained in stereoisomerically pure form or as a mixture of stereoisomers.

2. The method of claim 1, wherein in the carbonyl compound of general formula I
“a” represents a chemical double bond and Z represents-O or ═C(R⁴)—C(R⁵)═O; or
“a” represents a chemical single bond and Z represents-C(R⁵)=0;
wherein
R⁴ and R⁵ independently of each other represent H or lower alkyl.

3. The method of claim 1, wherein the carbonyl compound of general formula I possesses a labdane-type structure.

4. The method of claim 1, wherein R² represents a group Cyc-A-, wherein A represents a straight chain or branched C₁-C₄-alkylene bridge, and Cyc represents a mono- or polycyclic, saturated or unsaturated hydrocarbyl residue, wherein Cyc is optionally substituted with 1-10 substituents, which are independently selected from the group consisting of C₁-C₄-alkyl, C₁-C₄-alkylidene, C₂-C₄-alkenyl, oxo, hydroxy, and amino.

5. The method of claim 4, wherein Cyc-A represents a bicyclic residue of one of the formulae IIIa, IIIb, or IIIc

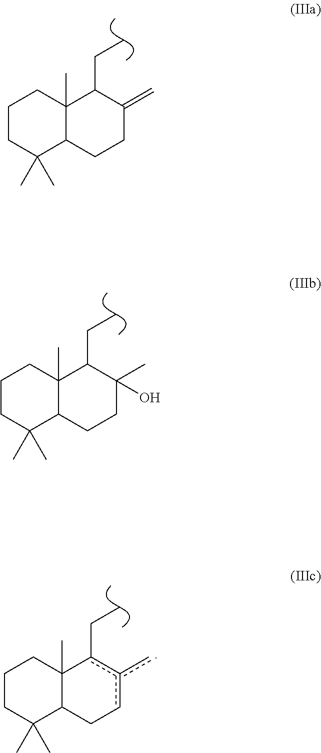

6. The method of claim 1, wherein the polypeptide having BVMO activity is selected from the group consisting of
(1) the group of polypeptides containing a flavin-containing monooxygenase (FMO) protein family domain having the Pfam ID number PF00743 within their amino acid sequence or a domain retaining at least 90% sequence identity to PF00743; and/or
(2) the group of polypeptides that comprise at least one sequence motif/domain selected from the group consisting of
GAGxSGL set forth in SEQ ID NO: 197;
EKNxxxxGTWxENRYPGCACDVPxHxYXXSFE set forth in SEQ ID NO: 198;
LxNAxGILNxWxxPxIPG set forth in SEQ ID NO: 199;
LxxKxVxxIGxGSSGIQIxPxI set forth in SEQ ID NO: 200;
GCRRxTPGxxYLExL set forth in SEQ ID NO: 201;
CATGFDxxxxPRFxxxG set forth in SEQ ID NO: 202;
PNxFxxxGPNxPxxNGxV set forth in SEQ ID NO: 203;
and
AxWPGSxLHYxEAxxxPRxED set forth in SEQ ID NO: 204;
wherein residues x represent independently of each other any natural amino acid residue;
and/or
(3) the group of polypeptides selected from the group consisting of
(a) polypeptides comprising the amino acid sequence of SCH23-BVMO1 set forth in SEQ ID NO:2;
(b) polypeptides comprising the amino acid sequence of SCH24-BVMO1 set forth in SEQ ID NO:6;
(c) polypeptides comprising the amino acid sequence of SCH25-BVMO1 set forth in SEQ ID NO:10;
(d) polypeptides comprising the amino acid sequence of SCH46-BVMO1 set forth in SEQ ID NO:13;
(e) polypeptides comprising the amino acid sequence of AspWeBVMO set forth in SEQ ID NO:16; and
(f) polypeptides comprising an amino acid sequence that has at least 70% identity to any one of the amino acid sequences of a) to e),

7. The method of claim 1, further comprising as step (3) processing of the carbonyl ester formed in step (1) or isolated in step (2) to obtain a derivative thereof using chemical or biocatalytic synthesis or a combination of both, wherein said derivative is optionally selected from the group consisting of a hydrocarbon, alcohol, diol, triol, acetal, ketal, aldehyde, acid, ether, amide, ketone, lactone, epoxide, acetate, glycoside and/or an ester, and optionally isolating the derivative of step (3).

8. The method of claim 7, wherein step (3) comprises the hydrolysis of the carbonyl ester compound with a polypeptide having esterase activity (EC 3.1.1.) to the corresponding de-esterified product, and optionally isolating the derivative of step (3); and optionally subjecting the de-esterified product in a further step (4) to an enzymatic redox reaction through the enzymatic action of a polypeptide having an alcohol dehydrogenase (ADH) (EC 1.1.1.-) activity.

9. An isolated polypeptide having BVMO activity as defined in claim 6.

10. An isolated nucleic acid molecule comprising a nucleic acid sequence encoding the polypeptide of claim 9.

11. An in vivo method for preparing labdane-type terpenes which method comprises providing a recombinant host expressing a set of polypeptides having enzymatic activities required for catalyzing the following sequence of reaction steps:
(1) optionally converting a labdane alcohol to the respective labdane aldehyde through the enzymatic action of an ADH polypeptide,
(2) converting said ladbane aldehyde of step (1) to the respective dinorlabdane carbonyl compound through the action of a polypeptide having enal-cleaving activity;
(3) optionally converting said dinorlabdane carbonyl compound of step (2) to the respective tetranorlabdanyl acetate through the action of a polypeptide having BVMO activity as defined in claim 9;
(4) optionally converting said tetranorlabdanyl acetate of step (3) to the respective tetranorlabdane alcohol through the action of a polypeptide having esterase activity; and optionally
(5) isolating the product of step (2), (3) or (4).

12. An in vivo method for preparing labdane-type cyclo-terpenes which method comprises providing a recombinant host expressing a set of polypeptides having enzymatic activities required for catalyzing the following sequence of reaction steps:
(1) optionally converting a labdane alcohol to the respective labdane aldehyde through the enzymatic action of an ADH polypeptide;
(2) converting said labdane aldehyde of step (1) to the respective labdane ester compound through the action of a polypeptide having BVMO activity as defined in claim 9;
(3) converting said labdane ester compound of step (2) to the respective norlabdane aldehyde, optionally through the action of a polypeptide having esterase activity;
(4) converting said norlabdane aldehyde of step (3) to the respective norlabdane ester through the action of the polypeptide having BVMO activity;
(5) converting said norlabdane ester of step (4) to the respective dinorlabdane alcohol through the action of a polypeptide having esterase activity;
(6) optionally converting said dinorlabdane alcohol of step (5) to the respective dinorlabdane carbonyl compound through the action of a polypeptide having ADH activity;
(7) optionally converting said dinorlabdane carbonyl compound of step (6) to the respective tetranorlabdanyl acetate through the action of the polypeptide having BVMO activity;
(8) converting said tetranorlabdanyl acetate of step (7) to the respective tetranorlabdane alcohol through the action of a polypeptide having esterase activity, and optionally
(9) isolating the product of step (5), (6), (7) or (8).


SEQUENCE LISTING (208 sequences)

SEQ ID NO: 1 - DNA, 1767 bp, Hyphozyma roseonigra
  source 1..1767: mol_type = other DNA
  CDS 1..1767
atgccttccgcaatcaccccgccggttgatcatcgcagtcttccaggtcttttcaagccacagaggaagctcaaagtgatatgtgtcggagccggcgcctcgggcttacttctttcctacaagatacaacgacacttcgaggatttcgagctccaagtctttgagaagaatcccgaggtatcaggaacctggtacgagaacaggtatcccggctgcgcttgtgatgttccctcgcataattatacatggtcttttgagcccaaaaccgactggtccgccaactatgcatcatcgaaggagattttcaaatatttcaaggacttcacgaggaaatatggtctaagcaagtacatcaagctggaacatgaggtcgtgggagccacgtggatggaggcggaggcacagtggaaagttgacgtcaaggaccttcgaagtggaaacacgcagagctcgtttgcgcatatactggtcaatgcaggaggcattttgaatgcttggcgatacccgccaattccaggaatcaaggatttcaagggtgatcttgttcactccgcagcttggccagaacatcttgatcttaatgggaaggttgtcggtctcatcggaaatggatcctccggcattcagatccttccggccatcaagaaggatgtaaagcaactcgttacattcattcgggaagcaacttgggtggcgcctcctttaggtcaagcctatcgtgcgttctcgactgatgaacaggctcagtttgcgcaagatccgcgccatcacctggagacacggcgtgcaactgaggctaccatgaatcaatcatttggtatcttccattcgggatccgaggagcagaaaggggttcgccaatatatgcagaatatcatggaaacgaagctcaacaacaaacagctcgagagtgtgctgattcctgagtggtcggtcggttgtcggcgtcttacaccaggtactaattatctagaatccctgtcagacgacaatgtcaaggtcgtctatggtgagatcacacaaattaccgaatcgggtgtcatctgcgatgatggtaaaggcgaatatcccgttgaagttcttatttgcgccaccggcttcgacaccaccttcaaaccacgattcccactcatcggtacaacccaagagaaactcagtgatgtttggaaagatgatccgaggggctactttgggatcgcaaccaacaactatcccaactacttcttcactcttggaccaaattgtccaatcggtaatggccccgtgctgtgtgccattgaagctgaggttgaatatataatcaacatgctctcgaagtttcagaaggagaacattcgttcttttgatatcaaggcagatgctgtcgacgccttcaacgactggaaggatgacttcatgaaagataccatctgggcagaacaatgccggtcatggtacaaggcaggatccgccaccggtaaaatccttgcattgtggccaggctcgactttgcactacttggaagcactcaagtcgccgcggtgggaggactgggacttcaagtatcagcctggtagaaatcgtttccactactttggaaatggtcatagctgtgctgagcaggatggcgatctgagctggtacattcgcaacgaggatgattcttatattgatccggtactcaagccgaagccgaaggcagcagttgaaagcgaggcacatatcgccctgccaggaatcggtccgatgttgatggaagacccgcgtgatgttgctgtagaggcctag

SEQ ID NO: 2 - protein, 588 aa, Hyphozyma roseonigra
MPSAITPPVDHRSLPGLFKPQRKLKVICVGAGASGLLLSYKIQRHFEDFELQVFEKNPEVSGTWYENRYPGCACDVPSHNYTWSFEPKTDWSANYASSKEIFKYFKDFTRKYGLSKYIKLEHEVVGATWMEAEAQWKVDVKDLRSGNTQSSFAHILVNAGGILNAWRYPPIPGIKDFKGDLVHSAAWPEHLDLNGKVVGLIGNGSSGIQILPAIKKDVKQLVTFIREATWVAPPLGQAYRAFSTDEQAQFAQDPRHHLETRRATEATMNQSFGIFHSGSEEQKGVRQYMQNIMETKLNNKQLESVLIPEWSVGCRRLTPGTNYLESLSDDNVKVVYGEITQITESGVICDDGKGEYPVEVLICATGFDTTFKPRFPLIGTTQEKLSDVWKDDPRGYFGIATNNYPNYFFTLGPNCPIGNGPVLCAIEAEVEYIINMLSKFQKENIRSFDIKADAVDAFNDWKDDFMKDTIWAEQCRSWYKAGSATGKILALWPGSTLHYLEALKSPRWEDWDFKYQPGRNRFHYFGNGHSCAEQDGDLSWYIRNEDDSYIDPVLKPKPKAAVESEAHIALPGIGPMLMEDPRDVAVEA

SEQ ID NO: 3 - DNA, 1767 bp, Hyphozyma roseonigra
  source 1..1767: mol_type = other DNA
atgccgtctgccattactccacctgttgatcaccgttccctgccgggcctgtttaaaccgcagcgcaagctgaaagtgatttgcgtgggcgcgggtgcgagcggcctgctgttgagctacaagattcagcgccacttcgaagatttcgagctgcaagtgtttgagaagaaccctgaagttagcggtacgtggtacgagaaccgttatccgggttgtgcgtgcgatgtgccgagccataactacacctggagctttgagccgaaaacggattggtccgccaattatgcgagcagcaaagagattttcaaatatttcaaagattttacgcgtaaatatggtctgtctaaatacattaaattggaacatgaagtggtcggcgcgacctggatggaagccgaggcgcagtggaaagttgacgttaaagatctgcgcagcggtaacacccagtccagcttcgcgcatatcctggttaacgccggcggcattctgaatgcctggcgttatccgccgattccgggcatcaaagatttcaagggtgacctggtgcatagcgcagcatggccggagcatttggacctgaatggcaaagtcgttggtctgatcggcaacggtagcagcggtatccaaatcctgccggcaattaagaaagacgtgaagcaactggtgacgtttatccgtgaagccacctgggtcgcaccgccgctgggtcaagcgtaccgtgcgttttccaccgacgagcaagcacagtttgcgcaggacccgcgccaccacctggaaacccgtcgtgcgaccgaagccaccatgaatcagagctttggtattttccatagcggcagcgaagaacagaaaggtgtccgccagtacatgcaaaacattatggaaaccaagctgaataataagcaactggagagcgtcctgattccggagtggagcgtcggctgtcgtcgtctgaccccgggcacgaactacctggaaagcctgagcgacgacaatgtcaaagttgtgtacggtgagattacccaaatcaccgagagcggtgtcatctgcgatgacggcaagggtgagtatccggttgaagtcctgatctgcgccaccggttttgatacgacctttaaaccgcgcttcccgctgatcggtacgacccaggaaaagctgagcgacgtgtggaaagatgatccgcgcggttacttcggcatcgcgacgaataattatccgaactatttcttcacgctgggtccgaactgcccgatcggtaatggcccggtcctgtgtgcgatcgaagccgaagttgagtacatcatcaacatgctgagcaagtttcagaaagaaaatattcgctccttcgacattaaagccgacgcggtggacgcgtttaatgattggaaagacgatttcatgaaagataccatctgggcagaacagtgccgtagctggtacaaggccggcagcgcgaccggcaagattctggcactgtggccgggcagcacgctgcactacctggaagcgctgaaaagcccgcgttgggaagattgggacttcaagtatcaaccgggccgtaaccgtttccactactttggcaacggtcacagctgtgccgagcaagatggcgacctgtcctggtacatccgtaatgaagatgacagctacattgacccggttctgaaaccgaagccgaaagccgcggtggagagcgaggcacacatcgcactgccgggtattggcccgatgctgatggaagatccgcgtgatgtcgcggttgaggcgtaa

SEQ ID NO: 4 - DNA, 1767 bp, Hyphozyma roseonigra
  source 1..1767: mol_type = other DNA
atgccatctgctatcactccaccagttgaccacagatctttgccaggtttgttcaagccacaaagaaagttgaaggttatctgtgttggtgctggtgcttctggtttgttgttgtcttacaagatccaaagacacttcgaagacttcgaattgcaagttttcgaaaagaacccagaagtttctggtacttggtacgaaaacagatacccaggttgtgcttgtgacgttccatctcacaactacacttggtctttcgaaccaaagactgactggtctgctaactacgcttcttctaaggaaatcttcaagtacttcaaggacttcactagaaagtacggtttgtctaagtacatcaagttggaacacgaagttgttggtgctacttggatggaagctgaagctcaatggaaggttgacgttaaggacttgagatctggtaacactcaatcttctttcgctcacatcttggttaacgctggtggtatcttgaacgcttggagatacccaccaatcccaggtatcaaggacttcaagggtgacttggttcactctgctgcttggccagaacacttggacttgaacggtaaggttgttggtttgatcggtaacggttcttctggtatccaaatcttgccagctatcaagaaggacgttaagcaattggttactttcatcagagaagctacttgggttgctccaccattgggtcaagcttacagagctttctctactgacgaacaagctcaattcgctcaagacccaagacaccacttggaaactagaagagctactgaagctactatgaaccaatctttcggtatcttccactctggttctgaagaacaaaagggtgttagacaatacatgcaaaacatcatggaaactaagttgaacaacaagcaattggaatctgttttgatcccagaatggtctgttggttgtagaagattgactccaggtactaactacttggaatctttgtctgacgacaacgttaaggttgtttacggtgaaatcactcaaatcactgaatctggtgttatctgtgacgacggtaagggtgaatacccagttgaagttttgatctgtgctactggtttcgacactactttcaagccaagattcccattgatcggtactactcaagaaaagttgtctgacgtttggaaggacgacccaagaggttacttcggtatcgctactaacaactacccaaactacttcttcactttgggtccaaactgtccaatcggtaacggtccagttttgtgtgctatcgaagctgaagttgaatacatcatcaacatgttgtctaagttccaaaaggaaaacatcagatctttcgacatcaaggctgacgctgttgacgctttcaacgactggaaggacgacttcatgaaggacactatctgggctgaacaatgtagatcttggtacaaggctggttctgctactggtaagatcttggctttgtggccaggttctactttgcactacttggaagctttgaagtctccaagatgggaagactgggacttcaagtaccaaccaggtagaaacagattccactacttcggtaacggtcactcttgtgctgaacaagacggtgacttgtcttggtacatcagaaacgaagacgactcttacatcgacccagttttgaagccaaagccaaaggctgctgttgaatctgaagctcacatcgctttgccaggtatcggtccaatgttgatggaagacccaagagacgttgctgttgaagcttaa

SEQ ID NO: 5 - DNA, 1839 bp, Filobasidium magnum
  source 1..1839: mol_type = other DNA
  CDS 1..1839
atgactatcgatttgcagcagcccgacgccgtgccattcacctcttcgacttttgtcgtgccggatccatcgaacctggcctctcaggcacagaattcacagctccaatctgctcaagaaggagcagagtaccctgtgaacgcacatggggttcgaggagatggaacgattcatgaacgaccgatcaacgatcgcaggaagatgcgcgtcatctgcgtcggtgcaggcatctcaggtctctatatggccatcaagctccctcgaagtacggaaaatgtagagctcaagatctacgagaagaatcacgatctcggtgggacctggctggagaataggtatccaggatgcgcttgtgatgtaccagcccatgcctacgcatacagcttcgagaacaaccccgaatttcctagattcttttcgagctcggaagacatccacaagtacttgcttcgcgtggctgataaatatgattgcaagaaatacatcgcattcaacaccaaagtagtcgaggccatttgggacgaagaacagggcatctataacgtcaagattgaacgctcggatggcacagtattccaggacacgtgcgaagttctattgaacgcttctggtatccttaacgcctggaggtaccctgggattcctggaattaaggactacaagggcacgttaatgcactcggctacctgggaccgatccgtgtccctgaaaggcaaaaaggttgccctcatcggatcaggatcatcaggcattcagatcttgcccaacatccttgacgattgcaaagaggtcgtgacatacatcattgatccagcctggattgcccctgcgaatcttgtcacggctggagtctcggacgacggtgaagagcctaaggagccgacgcctgaagaattggcgtcgagtagtgacttcgcctactcgcaagagcaaatcaatggcttcaagaaggaccctaagtcactgatggatcatcgagcaacgctcgaaaggacgatgaatcagtctttccccatcttactcagaggctcaccgtccaacctttatgccgcttctctctttgaagacctgatgaggaaacgccttgccaagaagcctgaggtagcggatgccatcatccccgaatggtcaatcggttgccgacgtctcactcctggaccacactatcttgaggccttgtgcaatcccaaggtcaagatcttgacccaagctatcaagtccttctccgataagggaatgtacactgccgatggcgaacacgaagactttgacgtggtgatatgcgcgactggattcgacgtatcgttccgaccccgattcaaatttatcggcaaggacgggtatgaggtgcccgagaactttggtcagactcccaaaggttacctcgctctcgcttacgccggtttccctaattcgttcatcttcatggggccgaacggacctatcgccaacggatctgtcgtggtctccctggagaaacaaggcgactacttcatcaaggcgatcaacaagatccaaaggcagaatataaaaggcatgactgtcagattcgatgcggtcgatgatttcaccaaccacgtagacaaatacatggataggaccgtgctcaccgatgactgcatcagctggtacaagaacgggaaacgagacggacgagtcagtgccgtctggcctgggagcgcacttcattatatggaggccatcgccgaccctagatgggaggattacacctacacttatcgcgaacccggtcattctttttcgttcttgggagatgggacgtcctgggtcgaacacaccggaggagacacggcttggtacctgaaagagaccctctaa

SEQ ID NO: 6 - protein, 612 aa, Filobasidium magnum
MTIDLQQPDAVPFTSSTFVVPDPSNLASQAQNSQLQSAQEGAEYPVNAHGVRGDGTIHERPINDRRKMRVICVGAGISGLYMAIKLPRSTENVELKIYEKNHDLGGTWLENRYPGCACDVPAHAYAYSFENNPEFPRFFSSSEDIHKYLLRVADKYDCKKYIAFNTKVVEAIWDEEQGIYNVKIERSDGTVFQDTCEVLLNASGILNAWRYPGIPGIKDYKGTLMHSATWDRSVSLKGKKVALIGSGSSGIQILPNILDDCKEVVTYIIDPAWIAPANLVTAGVSDDGEEPKEPTPEELASSSDFAYSQEQINGFKKDPKSLMDHRATLERTMNQSFPILLRGSPSNLYAASLFEDLMRKRLAKKPEVADAIIPEWSIGCRRLTPGPHYLEALCNPKVKILTQAIKSFSDKGMYTADGEHEDFDVVICATGFDVSFRPRFKFIGKDGYEVPENFGQTPKGYLALAYAGFPNSFIFMGPNGPIANGSVVVSLEKQGDYFIKAINKIQRQNIKGMTVRFDAVDDFTNHVDKYMDRTVLTDDCISWYKNGKRDGRVSAVWPGSALHYMEAIADPRWEDYTYTYREPGHSFSFLGDGTSWVEHTGGDTAWYLKETL

SEQ ID NO: 7 - DNA, 1839 bp, Filobasidium magnum
  source 1..1839: mol_type = other DNA
atgaccatcgatttgcaacagccagacgcagtcccgtttacgagcagcactttcgtcgtaccggacccgtccaacctggcatcccaggctcaaaacagccaactgcagagcgcgcaagagggcgcagagtacccggtgaatgcacacggtgtccgcggtgacggcaccattcacgagcgtccgatcaatgaccgtcgtaaaatgcgcgtcatctgcgttggtgcgggtattagcggcctgtatatggcgatcaaactgccgcgcagcaccgagaatgttgaactgaagatctacgagaaaaaccatgacctcggcggcacgtggctggagaatcgctaccctggctgcgcgtgcgatgttccggcgcatgcgtatgcatattcttttgagaataatccggaatttccacgctttttcagcagcagcgaggatatccacaagtacctgttgcgtgttgcggacaagtacgactgtaagaaatacatcgcctttaacaccaaagtcgttgaggctatctgggacgaagaacagggtatttacaatgtgaagattgagcgtagcgacggcaccgtgttccaggacacctgtgaggtgctgctgaacgcgagcggtattctgaatgcctggcgctacccgggcatccctggcattaaggattacaaaggtacgctgatgcacagcgctacctgggaccgtagcgtttctttgaaaggcaaaaaagtcgcactgattggcagcggtagcagcggtatccagattctgccgaacattctggacgactgcaaagaagtggtcacgtacattatcgacccggcgtggattgctccggctaacctggtgaccgcgggtgtctccgatgatggtgaggaaccgaaagagccaacccctgaggaactggcctcatcctccgacttcgcttatagccaggaacagattaacggcttcaagaaagatccgaagtcgctgatggatcaccgcgccacgctggagcgtaccatgaatcaatcgtttccgattctgctgcgtggctctccgagcaacttgtatgccgcaagcctgttcgaggatttgatgcgtaagcgtctggcgaagaagccggaagttgcggacgcgattatcccggagtggagcatcggttgcagacgcctgacgccgggtccgcattacctggaagcactgtgtaacccgaaagtgaagatcctgactcaggcgatcaagagctttagcgataagggcatgtatactgcggacggtgagcatgaagatttcgatgttgtcatttgtgcgaccggtttcgatgtgagctttcgtccgcgcttcaagtttattggtaaagatggctatgaagtcccagagaatttcggccaaacgccgaaaggttatctggcactggcgtacgccggcttcccgaacagcttcatctttatgggtccgaacggtccgattgcgaacggtagcgttgtggtgagcctggagaagcaaggtgactacttcattaaagcgatcaataagatccagcgtcaaaacattaagggtatgaccgttcgtttcgacgccgtggatgattttacgaatcacgtggacaaatacatggaccgtacggtgctgaccgacgattgcatcagctggtacaagaatggtaaacgtgacggtcgtgttagcgcagtttggccgggttccgcgctgcactatatggaagccatcgcagacccgcgttgggaagattacacctacacctatcgcgaaccgggtcactcttttagcttcctgggtgatggcaccagctgggttgagcatacgggtggcgataccgcctggtatttgaaagaaaccctgtaa

SEQ ID NO: 8 - DNA, 1839 bp, Filobasidium magnum
  source 1..1839: mol_type = other DNA
atgactatcgacttgcaacaaccagacgctgttccattcacttcttctactttcgttgttccagacccatctaacttggcttctcaagctcaaaactctcaattgcaatctgctcaagaaggtgctgaatacccagttaacgctcacggtgttagaggtgacggtactatccacgaaagaccaatcaacgacagaagaaagatgagagttatctgtgttggtgctggtatctctggtttgtacatggctatcaagttgccaagatctactgaaaacgttgaattgaagatctacgaaaagaaccacgacttgggtggtacttggttggaaaacagatacccaggttgtgcttgtgacgttccagctcacgcttacgcttactctttcgaaaacaacccagaattcccaagattcttctcttcttctgaagacatccacaagtacttgttgagagttgctgacaagtacgactgtaagaagtacatcgctttcaacactaaggttgttgaagctatctgggacgaagaacaaggtatctacaacgttaagatcgaaagatctgacggtactgttttccaagacacttgtgaagttttgttgaacgcttctggtatcttgaacgcttggagatacccaggtatcccaggtatcaaggactacaagggtactttgatgcactctgctacttgggacagatctgtttctttgaagggtaagaaggttgctttgatcggttctggttcttctggtatccaaatcttgccaaacatcttggacgactgtaaggaagttgttacttacatcatcgacccagcttggatcgctccagctaacttggttactgctggtgtttctgacgacggtgaagaaccaaaggaaccaactccagaagaattggcttcttcttctgacttcgcttactctcaagaacaaatcaacggtttcaagaaggacccaaagtctttgatggaccacagagctactttggaaagaactatgaaccaatctttcccaatcttgttgagaggttctccatctaacttgtacgctgcttctttgttcgaagacttgatgagaaagagattggctaagaagccagaagttgctgacgctatcatcccagaatggtctatcggttgtagaagattgactccaggtccacactacttggaagctttgtgtaacccaaaggttaagatcttgactcaagctatcaagtctttctctgacaagggtatgtacactgctgacggtgaacacgaagacttcgacgttgttatctgtgctactggtttcgacgtttctttcagaccaagattcaagttcatcggtaaggacggttacgaagttccagaaaacttcggtcaaactccaaagggttacttggctttggcttacgctggtttcccaaactctttcatcttcatgggtccaaacggtccaatcgctaacggttctgttgttgtttctttggaaaagcaaggtgactacttcatcaaggctatcaacaagatccaaagacaaaacatcaagggtatgactgttagattcgacgctgttgacgacttcactaaccacgttgacaagtacatggacagaactgttttgactgacgactgtatctcttggtacaagaacggtaagagagacggtagagtttctgctgtttggccaggttctgctttgcactacatggaagctatcgctgacccaagatgggaagactacacttacacttacagagaaccaggtcactctttctctttcttgggtgacggtacttcttgggttgaacacactggtggtgacactgcttggtacttgaaggaaactttgtaa

SEQ ID NO: 9 - DNA, 1767 bp, Papiliotrema laurentii
  source 1..1767: mol_type = other DNA
  CDS 1..1767
atgccttccgcaatcaccccgccggttgatcatcgcagtcttccaggtcttttcaagccacagaggaagctcaaagtgatatgtgtcggagccggcgcctcgggcttacttctttcctacaagatacaacgacacttcgaggatttcgagctccaagtctttgagaagaatcctgaagtatcaggaacctggtacgagaacagatatcccggctgcgcttgtgatgttccctcgcataattatacatggtcttttgagcccaaaaccgactggtccgccaactatgcatcatcgaaggagattttcaaatatttcaaggacttcacgaagaagtatggtcttagcaagtacatcaagctggagcatgaggtcgtgggggccacgtggatggaggcggaggcacagtggaaagttgacgtcaaggaccttcgaagtggaaacacgcagagctcgtttgcgcatatactggtcaatgcaggaggcattctgaatgcttggcgatatccgccaattccaggaatcaaggatttcaagggtgatcttgtccactccgcagcttggccagaacatcttgatcttaatgggaaggttgtcggtctcatcggaaatggatcctccggcattcagatccttccggccatcaagaaggatgtaaagcaactcgttacattcattcgggaagcaacttgggtggcgcctcctttaggtcaagcctatcgtgcgttctcgactgatgaacaggctcagtttgcgcaagatccgcgccatcacctggagacacggcgtgcaattgaggctaccatgaatcaatcatttggtatcttccattcgggatccgaggagcagaaaggggttcgccaatatatgcagaatatcatggaaacgaagctcaacaacaaacagctcgagagtgtgctgattcctgagtggtcggtcggttgtcggcgtcttacaccaggtactaattacctagaatccctgtcggacgacaatgtcaaggtcgtctacggtgagatcacacaaattaccgaattgggtgtcatctgcgatgatggcaaaggcgagtatcccgttgaagttcttatttgcgccactggcttcgacaccaccttcaaaccacgattcccactcatcggtacaacccaagagaaactcagtgatgtttggaaagatgatccgaggggttacttcgggattgcaaccaacaactatcccaactacttcttcactcttggaccgaattgtccaatcggtaatggccccgtgctgtgtgccatcgaagctgaggttgattatataatcaacatgctctcaaagtttcaaatggagaacattcgttcttttgatatcaaggcagatgctgtcgacgccttcaacgactggaaggatgacttcatgaaagataccatctgggcagaacaatgccggtcatggtacaaggcaggatctgccaccggtaaaatccttgcattgtggccaggctcgactttgcactacttggaagcactcaagtcgccgcggtgggaggattgggacttcaagtatcagcctggtagaaatcgtttccactactttggaaatggtcatagctgtgctgagcaggatggcgatctgagctggtacattcgcaacgaggatgattcttatattgatccggtactcaagccaaagtcgaaggcagcaattgagagcgaggcacatatcgccctgccaggaatcggtccgatgttgatggaagacccgcgtgatgttgctgtagaggcctag

SEQ ID NO: 10 - protein, 588 aa, Papiliotrema laurentii
MPSAITPPVDHRSLPGLFKPQRKLKVICVGAGASGLLLSYKIQRHFEDFELQVFEKNPEVSGTWYENRYPGCACDVPSHNYTWSFEPKTDWSANYASSKEIFKYFKDFTKKYGLSKYIKLEHEVVGATWMEAEAQWKVDVKDLRSGNTQSSFAHILVNAGGILNAWRYPPIPGIKDFKGDLVHSAAWPEHLDLNGKVVGLIGNGSSGIQILPAIKKDVKQLVTFIREATWVAPPLGQAYRAFSTDEQAQFAQDPRHHLETRRAIEATMNQSFGIFHSGSEEQKGVRQYMQNIMETKLNNKQLESVLIPEWSVGCRRLTPGTNYLESLSDDNVKVVYGEITQITELGVICDDGKGEYPVEVLICATGFDTTFKPRFPLIGTTQEKLSDVWKDDPRGYFGIATNNYPNYFFTLGPNCPIGNGPVLCAIEAEVDYIINMLSKFQMENIRSFDIKADAVDAFNDWKDDFMKDTIWAEQCRSWYKAGSATGKILALWPGSTLHYLEALKSPRWEDWDFKYQPGRNRFHYFGNGHSCAEQDGDLSWYIRNEDDSYIDPVLKPKSKAAIESEAHIALPGIGPMLMEDPRDVAVEA

SEQ ID NO: 11 - DNA, 1767 bp, Papiliotrema laurentii
  source 1..1767: mol_type = other DNA
atgccatctgccattactccacctgttgatcatcgtagcctgccgggtctgttcaagccgcaacgtaagttgaaagtgatctgtgttggcgcgggcgcgagcggcctgttgctgagctacaagattcagcgtcactttgaggactttgagttgcaagtttttgagaaaaaccctgaagtgagcggcacctggtacgagaatcgctacccgggttgcgcgtgcgatgttccgagccataactatacctggtcttttgagccgaaaacggattggtccgcaaactatgccagcagcaaagaaattttcaagtacttcaaagatttcaccaagaaatatggtctgtctaaatacattaaactggaacacgaagtcgtgggtgcgacgtggatggaagcggaagctcaatggaaagttgacgtcaaagacttgcgtagcggcaacacccagagctccttcgcgcacattctggtcaatgccggtggcattctgaacgcttggcgttacccgccgattccgggtatcaaagattttaagggtgacctggtgcactcggcagcgtggccggagcatctggatctgaatggtaaagtcgttggcctgattggtaacggtagcagcggcatccaaattctgccggccatcaaaaaagacgtgaaacaactggtcacgtttatccgtgaggccacgtgggtcgccccgccgctgggccaagcgtaccgcgcatttagcaccgacgaacaggcgcagtttgcacaagacccgcgtcaccatctggaaactcgtcgcgcgattgaagctaccatgaatcagagcttcggtatcttccacagcggttcagaggaacagaaaggtgtgcgtcagtacatgcagaatatcatggaaacgaaattgaataacaaacagctggagagcgtgctgattccggagtggtccgtgggttgtcgccgtctgaccccgggcacgaactatctggagagcttgagcgacgataacgtgaaagttgtttatggcgagatcacccagatcaccgagctgggtgtgatttgcgatgatggcaagggtgagtacccggtcgaagtgctgatttgcgctaccggtttcgacaccacgttcaaaccgcgcttcccgttgattggcaccacccaggaaaagctgagcgacgtctggaaagatgaccctcgcggttatttcggtatcgcgaccaataactacccgaactactttttcaccctgggtccgaactgcccgatcggcaatggtccggtcctgtgtgcaatcgaagctgaagtggactatatcatcaatatgctgagcaaatttcagatggaaaacattcgcagcttcgacattaaagccgacgcagttgatgcgtttaacgactggaaagatgactttatgaaagacaccatctgggcagagcagtgtcgttcttggtacaaggctggttctgcgacgggtaagattttggcactgtggccgggcagcacgctgcattatctggaagccctgaaaagcccacgctgggaagattgggacttcaagtatcaaccgggtcgtaatcgctttcactacttcggtaacggccacagctgcgcggagcaagatggtgatctgtcctggtatatccgtaatgaagatgacagctacattgacccggtactgaagccgaagtccaaggcagcgatcgagagcgaagcacacatcgcgctgccaggcattggtccgatgctgatggaggacccgcgtgacgttgcggttgaggcataa

SEQ ID NO: 12 - DNA, 1767 bp, Bensingtonia ciliata
  source 1..1767: mol_type = other DNA
  CDS 1..1767
atgccttccgcaatcaccccaccggttgatcatcgcagtcttccaggtcttttcaagccacagaggaagctcaaagtgatatgtgtcggagccggcgcctcgggcttacttctttcctacaagatacaacgacacttcgaggatttcgagctccaagtctttgagaagaatcctgaggtatcaggaacctggtacgagaacaggtatcccggctgtgcttgtgatgttccctcgcataattatacatggtcttttgagcccaaaaccgactggtccgccaactatgcatcatcgaaggagattttcaaatatttcaaggacttcacgaagaagtatggtcttagcaagtacatcaagctggagcatgaggtcgtgggggccacgtggatggaggcggaggcacagtggaaagttgacgtcaaggaccttcgaagtggaaacacgcagagctcgtttgcgcatatactggtcaatgcaggaggcattctgaatgcttggcgatatccgccaattccaggaatcaaggatttcaagggtgatcttgtccactccgcagcttggccagaacatcttgatcttaatgggaaggttgtcggtctaatcggaaatggatcctccggcattcagatccttccggccatcaagaaggatgtaaagcaactcgttacatttattcgggaagcaacttgggtggcgcctcctttaggtcaagcctatcgtgcgttctcgactgatgaacaggctcagtttgcgcaagatccgcgccatcacctggaaacacgtcgtgcaactgaggctaccatgaatcaatcatttggtatcttccattcgggatccgaggagcagaaaggagttcgccaatatatgcaggatatcatggaaacgaagctcaacaacaaacagctcgagagtgtgctgattcctgagtggtcggtcggttgtcggcgtcttacaccaggtactaattacctagaatccctatcggacgacaatgtcaaggtcgtctacggtgaaatcacacaaattaccgaatcaggtgtcatctgcgatgatggtaaaggcgaatatcccgtcgaagttcttatttgcgccaccggcttcgacaccaccttcaaaccacgatttccactcatcggcactacgaaagagaagctcagtgatgtttggaaagatgatccgaggggctactttgggatcgcaaccaacaactatcccaactacttcttcactcttggaccgaattgtccaatcggtaatggccccgtgctgtgtgccattgaagctgaggttgaatatataatcaacatgctctcgaagtttcagaaggagaacattcgttcttttgatatcaaggcagatgctgtcgacgccttcaacgactggaaggatgacttcatgaaagataccatctgggcagaacaatgccggtcatggtacaaggcaggatccgccactggtaaaatccttgcattgtggccaggctcgactttgcactacttggaagcactcaagtcgccgcggtgggaggactgggacttcaagtatcagcctggtagaaatcgtttccattactttggaaatggtcatagctgtgctgagcaggatggcgatctgagctggtacatccgcaacgaggatgattcttatattgatccggtactcaagccaaagccgaaggcagcagttgagagcgaggcacatatcgccctgccaggaatcggtccgatgttgatggaagacccgcgtgatgttgctgtagaggcctag

SEQ ID NO: 13 - protein, 588 aa, Bensingtonia ciliata
MPSAITPPVDHRSLPGLFKPQRKLKVICVGAGASGLLLSYKIQRHFEDFELQVFEKNPEVSGTWYENRYPGCACDVPSHNYTWSFEPKTDWSANYASSKEIFKYFKDFTKKYGLSKYIKLEHEVVGATWMEAEAQWKVDVKDLRSGNTQSSFAHILVNAGGILNAWRYPPIPGIKDFKGDLVHSAAWPEHLDLNGKVVGLIGNGSSGIQILPAIKKDVKQLVTFIREATWVAPPLGQAYRAFSTDEQAQFAQDPRHHLETRRATEATMNQSFGIFHSGSEEQKGVRQYMQDIMETKLNNKQLESVLIPEWSVGCRRLTPGTNYLESLSDDNVKVVYGEITQITESGVICDDGKGEYPVEVLICATGFDTTFKPRFPLIGTTKEKLSDVWKDDPRGYFGIATNNYPNYFFTLGPNCPIGNGPVLCAIEAEVEYIINMLSKFQKENIRSFDIKADAVDAFNDWKDDFMKDTIWAEQCRSWYKAGSATGKILALWPGSTLHYLEALKSPRWEDWDFKYQPGRNRFHYFGNGHSCAEQDGDLSWYIRNEDDSYIDPVLKPKPKAAVESEAHIALPGIGPMLMEDPRDVAVEA

SEQ ID NO: 14 - DNA, 1767 bp, Bensingtonia ciliata
  source 1..1767: mol_type = other DNA
atgccatctgccattactccacctgttgatcatcgtagcctgccgggtctgttcaagccgcaacgtaagttgaaagtgatctgtgttggcgcgggcgcgagcggcctgttgctgagctacaagattcagcgtcactttgaggactttgagttgcaagtttttgagaaaaaccctgaagtgagcggcacctggtacgagaatcgctacccgggttgcgcgtgcgatgttccgagccataactatacctggtcttttgagccgaaaacggattggtccgcaaactatgccagcagcaaagaaattttcaagtacttcaaagatttcaccaagaaatatggtctgtctaaatacattaaactggaacacgaagtcgtgggtgcgacgtggatggaagcggaagctcaatggaaagttgacgtcaaagacttgcgtagcggcaacacccagagctccttcgcgcacattctggtcaatgccggtggcattctgaacgcttggcgttacccgccgattccgggtatcaaagattttaagggtgacctggtgcactcggcagcgtggccggagcatctggatctgaatggtaaagtcgttggcctgattggtaacggtagcagcggcatccaaattctgccggccatcaaaaaagacgtgaaacaactggtcacgtttatccgtgaggccacgtgggtcgccccgccgctgggccaagcgtaccgcgcatttagcaccgacgaacaggcgcagtttgcacaagacccgcgtcaccatctggaaactcgtcgcgcgaccgaagctaccatgaatcagagcttcggtatcttccacagcggttcagaggaacagaaaggtgtgcgtcagtacatgcaggatatcatggaaacgaaattgaataacaaacagctggagagcgtgctgattccggagtggtccgtgggttgtcgccgtctgaccccgggcacgaactatctggagagcttgagcgacgataacgtgaaagttgtttatggcgagatcacccagatcaccgagtccggtgtgatttgcgatgatggcaagggtgagtacccggtcgaagtgctgatttgcgctaccggtttcgacaccacgttcaaaccgcgcttcccgttgattggcaccaccaaagaaaagctgagcgacgtctggaaagatgaccctcgcggttatttcggtatcgcgaccaataactacccgaactactttttcaccctgggtccgaactgcccgatcggcaatggtccggtcctgtgtgcaatcgaagctgaagtggagtatatcatcaatatgctgagcaaatttcagaaagaaaacattcgcagcttcgacattaaagccgacgcagttgatgcgtttaacgactggaaagatgactttatgaaagacaccatctgggcagagcagtgtcgttcttggtacaaggctggttctgcgacgggtaagattttggcactgtggccgggcagcacgctgcattatctggaagccctgaaaagcccacgctgggaagattgggacttcaagtatcaaccgggtcgtaatcgctttcactacttcggtaacggccacagctgcgcggagcaagatggtgatctgtcctggtatatccgtaatgaagatgacagctacattgacccggtactgaagccgaagccgaaggcagcggtggagagcgaagcacacatcgcgctgccaggcattggtccgatgctgatggaggacccgcgtgacgttgcggttgaggcataa

SEQ ID NO: 15 - DNA, 1674 bp, Aspergillus wentii
  source 1..1674: mol_type = other DNA
  CDS 1..1674
atgaccaaagacaataccacatcattcccctcgcacgccatctacgagccacgccggacattaaaagtgctggtcataggggctggtgcgtccggtctattattagcatacaaactacagcggcactttgattgtgtggaaatcacggtgtttgagaagaaccccgcagtgtccggcacttggtttgagaatcgatatccgggatgtgcctgtgacgttccttcgcattgctatacatggtccttcgagcccaaccccaactggtccgccaactacgctggagccgacgagattcgacaatactttgtcgatttctgccatcgccacgacttgcagaaatatatccatctggaacatgaggtggtccacgcagcgtggaagtcggagactggccactgggaggtgcaagtgcgcgatatacaacacaattctcacacacagcatactgcgcatatcttgattaatgctactggaatactgaatcaatggaagtggccatccattcccggattacagtcgttccagggagatcttttgcacagtgcagcatgggactcgtcagtcaatctagaggataaaacggtcgctgtcattggaaacggatcatccggaatccagattgtcccagcgattctaccccaagtgcgcaaactcgtgcactttactcgtcaagcggcatgggtcgcacctccagtcaatgaagagtatcaggaatactcgcccgaacagatcgaacgctttcgctcagacccaacatacctgcttggggttcgtcgacagattgaagcacggatgaacggctcatttctgaaattcatccaaggctcagacatgcaacgtcgtgcacacgagtatgtcatgctgcacatgatgaagagactggacggagacgcctccctggcagagaccttggtaccaaccttcccatttggctgtcgaagaccgacgccaggaaccgggtatctcgaagcactgaaggactcgaaagtggaaacaattaccggagcccgaatcgcgaatgtgacgggtaaccaggtggtcctcgagaatggcacgtcgtatacggtggatgcgattgtgtgcgccacgggattcgatacgtcttacaaaccacgattcccactggtcggcagagacagcaccactctcagcgaggcctggaaggacgaagtgtctgcatatctggggcttacagttcctggatttcccaactatttttccatcttgggaccgaactgtccggtgggtaacgggccggtgttgatcagtatcgaaaaacaggtcgaatatattgttcaggtactggggaaaatgcagaaggagaatctacagtcatttgaagtccggcggacggcaacagactcgtttaaccaatggaaggatgcattcatgcaaaacacggtgtggacgagtggttgtcgcagctggtatcagaatggctcgaaagggaaccagatcgtggctctctggcctggatccacgttgcactatttggaggcgattcagcatccacgatacgaggactacatctggaccagtccacctggtgtcaatccatgggcctttctaggcaacgggcagagtacggccgaaacccgtcccggaggcgacacgagttggtatctgcgttcgaaagatgattcatttatagatccatgtctgagacagctttag

SEQ ID NO: 16 - protein, 557 aa, Aspergillus wentii
MTKDNTTSFPSHAIYEPRRTLKVLVIGAGASGLLLAYKLQRHFDCVEITVFEKNPAVSGTWFENRYPGCACDVPSHCYTWSFEPNPNWSANYAGADEIRQYFVDFCHRHDLQKYIHLEHEVVHAAWKSETGHWEVQVRDIQHNSHTQHTAHILINATGILNQWKWPSIPGLQSFQGDLLHSAAWDSSVNLEDKTVAVIGNGSSGIQIVPAILPQVRKLVHFTRQAAWVAPPVNEEYQEYSPEQIERFRSDPTYLLGVRRQIEARMNGSFLKFIQGSDMQRRAHEYVMLHMMKRLDGDASLAETLVPTFPFGCRRPTPGTGYLEALKDSKVETITGARIANVTGNQVVLENGTSYTVDAIVCATGFDTSYKPRFPLVGRDSTTLSEAWKDEVSAYLGLTVPGFPNYFSILGPNCPVGNGPVLISIEKQVEYIVQVLGKMQKENLQSFEVRRTATDSFNQWKDAFMQNTVWTSGCRSWYQNGSKGNQIVALWPGSTLHYLEAIQHPRYEDYIWTSPPGVNPWAFLGNGQSTAETRPGGDTSWYLRSKDDSFIDPCLRQL

SEQ ID NO: 17 - DNA, 1674 bp, Aspergillus wentii
  source 1..1674: mol_type = other DNA
atgaccaaagataacaccacgtcctttccgagccacgccatttacgagccgcgccgtaccctgaaagtcctggtgatcggtgctggcgcgagcggtttgttgctggcatataagctgcagcgccacttcgattgcgttgagattaccgtattcgagaagaatccggcagtcagcggcacctggtttgagaatcgttaccctggttgtgcatgtgacgtgccgagccattgctacacctggtcgttcgagccaaacccgaattggagcgcaaactacgcgggtgcggatgaaattcgccagtatttcgttgatttctgtcaccgtcatgatctgcagaagtacatccatctggagcacgaagtcgttcatgcggcatggaaatcggagactggtcactgggaagtgcaagtccgtgacatccagcacaacagccatacccagcacacggcgcacattttgatcaacgcaacgggtatcctgaatcaatggaaatggccgagcattccgggcctgcagagctttcagggtgatctgctgcatagcgcagcgtgggacagctccgtcaacttagaggataagaccgtcgcggtgatcggtaatggcagcagcggtatccagattgtgccggccatcctgccgcaagtgcgcaaactggttcactttacgcgtcaagcggcatgggtggcaccgccggtgaacgaagagtaccaagagtacagcccggagcaaattgagcgtttccgtagcgacccgacctacctgttgggcgtccgccgtcaaattgaagcccgtatgaacggcagctttctgaagtttattcagggcagcgacatgcagcgcagagcgcacgaatacgttatgctgcacatgatgaagcgtctggacggtgatgcgagccttgctgagactctggtgccgacgtttccgttcggctgccgtcgtccgaccccgggcaccggttatctggaagcgctgaaagactctaaagttgaaacgatcacgggtgcccgtatcgcaaatgttacgggcaaccaagttgtcctggagaacggtactagctatacggtcgatgctattgtctgtgctaccggtttcgacaccagctataagccgcgtttcccgctggttggccgcgactctaccaccctgagcgaagcctggaaagacgaagtgtctgcgtacctgggtctgaccgttccgggttttccgaactatttcagcatcctgggtcctaattgcccggtcggtaatggtccggttttgatcagcatcgagaaacaagtggagtatatcgtgcaagttctgggtaagatgcagaaagaaaacttgcagtccttcgaagttcgccgtaccgccaccgacagcttcaatcagtggaaagatgcgttcatgcaaaacacggtgtggacctcaggttgccgttcttggtatcagaatggcagcaagggcaaccaaattgtcgcgctgtggccgggttccacgctgcactacctggaagcgattcaacatcctcgctacgaagattatatctggacgagcccaccgggtgttaatccgtgggcgtttctgggcaatggccagagcaccgcggaaacccgtccgggtggcgacacttcctggtatctccgctccaaagatgacagctttattgacccatgcctgcgtcagctgtaa

SEQ ID NO: 18 - DNA, 1674 bp, Aspergillus wentii
  source 1..1674: mol_type = other DNA
atgactaaggacaacactacttctttcccatctcacgctatctacgaaccaagaagaactttgaaggttttggttatcggtgctggtgcttctggtttgttgttggcttacaagttgcaaagacacttcgactgtgttgaaatcactgttttcgaaaagaacccagctgtttctggtacttggttcgaaaacagatacccaggttgtgcttgtgacgttccatctcactgttacacttggtctttcgaaccaaacccaaactggtctgctaactacgctggtgctgacgaaatcagacaatacttcgttgacttctgtcacagacacgacttgcaaaagtacatccacttggaacacgaagttgttcacgctgcttggaagtctgaaactggtcactgggaagttcaagttagagacatccaacacaactctcacactcaacacactgctcacatcttgatcaacgctactggtatcttgaaccaatggaagtggccatctatcccaggtttgcaatctttccaaggtgacttgttgcactctgctgcttgggactcttctgttaacttggaagacaagactgttgctgttatcggtaacggttcttctggtatccaaatcgttccagctatcttgccacaagttagaaagttggttcacttcactagacaagctgcttgggttgctccaccagttaacgaagaataccaagaatactctccagaacaaatcgaaagattcagatctgacccaacttacttgttgggtgttagaagacaaatcgaagctagaatgaacggttctttcttgaagttcatccaaggttctgacatgcaaagaagagctcacgaatacgttatgttgcacatgatgaagagattggacggtgacgcttctttggctgaaactttggttccaactttcccattcggttgtagaagaccaactccaggtactggttacttggaagctttgaaggactctaaggttgaaactatcactggtgctagaatcgctaacgttactggtaaccaagttgttttggaaaacggtacttcttacactgttgacgctatcgtttgtgctactggtttcgacacttcttacaagccaagattcccattggttggtagagactctactactttgtctgaagcttggaaggacgaagtttctgcttacttgggtttgactgttccaggtttcccaaactacttctctatcttgggtccaaactgtccagttggtaacggtccagttttgatctctatcgaaaagcaagttgaatacatcgttcaagttttgggtaagatgcaaaaggaaaacttgcaatctttcgaagttagaagaactgctactgactctttcaaccaatggaaggacgctttcatgcaaaacactgtttggacttctggttgtagatcttggtaccaaaacggttctaagggtaaccaaatcgttgctttgtggccaggttctactttgcactacttggaagctatccaacacccaagatacgaagactacatctggacttctccaccaggtgttaacccatgggctttcttgggtaacggtcaatctactgctgaaactagaccaggtggtgacacttcttggtacttgagatctaaggacgactctttcatcgacccatgtttgagacaattgtaa

SEQ ID NO: 19 - DNA, 1038 bp, Hyphozyma roseonigra
  source 1..1038: mol_type = other DNA
  CDS 1..1038
atgccttccgatcttccccgaccagcatatgacccggaaatagagcccttcctctctatggtcccattaccaccaacaatcaatgcggatatcatgaaagaattgcgtaaagcacctctgctcagtcaagcgcctgacctcgacgcattactttccgacaagccaataactcaccgcgaagtcagcattccaggtctcaattcccaagatccacaaatcacgttgtcaatattctccagtacattggagggtggcccgaaaccatgtatctatttcgttcatggtggcggtatgatcatcggatgtcgattcgtgggtattgaggattatcttcaatacgtcgagcagaacgacgctgtcgtcgtggctgtagagtatcgtctcgctccggaacacccggacccagcgcctgtcaatgattgttacgctggacttttatggacggcagcaaatgctgcagagctaggcatcgatctggagagactgttgatctgtggcgcttctgctggtggtggtctttctgctggagtggcattgatggcacgagacaagaaaggtccaaaattggtaggacaattgttatgctatccaatgctcgacgataggaatgattcactctcaagtcagcagtacgtggatgaaggtgtttggagtcgtggtagcaatgcatttggctggaagcaattgcttggagacagggcgggcaaagagggagtcagtatttatgctgcgccggcaagagcaactgatttgagcggactgccgaacactttcatcgacgttggcagcgctgaagtcttcagggatgaggacatcgcttatgcctcgaggttatgggctgttggtgtccaagcggaacttcatgtgtggccgggtggatatcatgctgcggagaacatggcacctgggactgattactctaagaaggtgaaagcgactcgcttggcatggatgaagagagtcttcatgaaagccccaaagtcgacgacagagtcgttgcctgctccaacagtggatgaagctgttggcacaatatga

SEQ ID NO: 20 - protein, 345 aa, Hyphozyma roseonigra
MPSDLPRPAYDPEIEPFLSMVPLPPTINADIMKELRKAPLLSQAPDLDALLSDKPITHREVSIPGLNSQDPQITLSIFSSTLEGGPKPCIYFVHGGGMIIGCRFVGIEDYLQYVEQNDAVVVAVEYRLAPEHPDPAPVNDCYAGLLWTAANAAELGIDLERLLICGASAGGGLSAGVALMARDKKGPKLVGQLLCYPMLDDRNDSLSSQQYVDEGVWSRGSNAFGWKQLLGDRAGKEGVSIYAAPARATDLSGLPNTFIDVGSAEVFRDEDIAYASRLWAVGVQAELHVWPGGYHAAENMAPGTDYSKKVKATRLAWMKRVFMKAPKSTTESLPAPTVDEAVGTI

SEQ ID NO: 21 - DNA, 1038 bp, Hyphozyma roseonigra
  source 1..1038: mol_type = other DNA
atgccatcggatctgccgcgcccagcctacgaccctgaaatcgaaccgttcttgagcatggttccgctgcctccgaccattaacgcggacattatgaaagaactgcgtaaggccccactgctgagccaggctccggatctggatgccctgctgagcgacaagccgattactcaccgtgaggtgtccatcccgggtctgaacagccaggacccgcagattaccctgagcatctttagctctaccctggagggtggcccgaagccgtgtatctacttcgtgcacggtggcggcatgattattggctgtcgcttcgtcggtattgaggactacttgcaatacgtggaacagaatgacgcggtcgttgtggccgttgagtatcgtctggcaccggaacatccggacccggcaccggtgaatgactgctacgcgggtctgctgtggaccgctgcgaacgcggcagaactgggcatcgatttggagcgtctgctgatctgcggcgcttctgcgggtggcggtctgtcagcgggtgtggcgctgatggcacgcgacaaaaagggtccgaaactggtcggtcagctgctgtgctatccgatgctcgacgatcgtaacgatagcttgagcagccagcaatacgtagatgagggtgtttggagccgtggtagcaatgcgtttggttggaagcaactgctgggtgatcgtgccggcaaagagggcgtgtccatttacgcggcaccggctcgcgcaaccgacctgtctggcttgcctaacacgtttatcgacgttggttccgccgaggttttccgtgatgaagatatcgcgtatgcgagccgcttatgggcagtcggtgttcaagcggagctgcatgtctggccgggtggttatcacgctgcggagaatatggcaccgggcaccgattatagcaaaaaagtcaaggcgacgcgtctggcatggatgaaacgcgtctttatgaaggccccgaaaagcaccacggagagcctgccggcaccgacggttgacgaagcggtgggcacgatctaa

SEQ ID NO: 22 - DNA, 1038 bp, Hyphozyma roseonigra
  source 1..1038: mol_type = other DNA
atgccatctgacttgccaagaccagcttacgacccagaaatcgaaccattcttgtctatggttccattgccaccaactatcaacgctgacatcatgaaggaattgagaaaggctccattgttgtctcaagctccagacttggacgctttgttgtctgacaagccaatcactcacagagaagtttctatcccaggtttgaactctcaagacccacaaatcactttgtctatcttctcttctactttggaaggtggtccaaagccatgtatctacttcgttcacggtggtggtatgatcatcggttgtagattcgttggtatcgaagactacttgcaatacgttgaacaaaacgacgctgttgttgttgctgttgaatacagattggctccagaacacccagacccagctccagttaacgactgttacgctggtttgttgtggactgctgctaacgctgctgaattgggtatcgacttggaaagattgttgatctgtggtgcttctgctggtggtggtttgtctgctggtgttgctttgatggctagagacaagaagggtccaaagttggttggtcaattgttgtgttacccaatgttggacgacagaaacgactctttgtcttctcaacaatacgttgacgaaggtgtttggtctagaggttctaacgctttcggttggaagcaattgttgggtgacagagctggtaaggaaggtgtttctatctacgctgctccagctagagctactgacttgtctggtttgccaaacactttcatcgacgttggttctgctgaagttttcagagacgaagacatcgcttacgcttctagattgtgggctgttggtgttcaagctgaattgcacgtttggccaggtggttaccacgctgctgaaaacatggctccaggtactgactactctaagaaggttaaggctactagattggcttggatgaagagagttttcatgaaggctccaaagtctactactgaatctttgccagctccaactgttgacgaagctgttggtactatctaa

SEQ ID NO: 23 - DNA, 933 bp, Filobasidium magnum
  source 1..933: mol_type = other DNA
  CDS 1..933
atgactcatagccctccactcgatgccgaactttcgctactccgatatgctcctgctgttcccgtgggatggcagttgggacgaaaactcttgcggatgaacacactcatgacgcgccctatggagggtgtcatgcgagatgatgtggtcataccaaatcttgatggtactgccaacatcagactgttcatttgtcgccctcaagaccctactgagactatgccggtgatactttggttacacggaggcggtatggtcgcaggtcattacaaacaagactccgggttcatggacatctgggccaagcgcctaggagcctttgtggtttcggtcgattatcgtctggctcccgaggccaaggctccagccgctctagacgattgcatcgctgcttggcaatggatcaccacgcagaccgctcgaggcatcgacactacccgcatggcggtgggtggtgcgagcgcaggaggaggcctggcggccagtaccgttcagcgacttgtcgatctcggaggagtgaaacctgtctttcaattgctcatctatcccatgttggacgacaggacggtggtcagatttgatcccgaccgaagatattacatgtggacaccggattgtaatcgatatggctggacctcgtacctcggagtccctccagggagcgctgaggtgcctccctatgcgtcggcggcacgtcgaccggatctatcaggtctacctcccacctggatcggtgttgggtcactggatctctttcacgacgaggacatggattatgcgcgcaggttacgtgagagcggagttccggttgaggaatatgtcgctgtcggagcgcctcatgccttcgacacgatatatggaaaggcgaaggtcaccttggatttctgggactcgcatttcaacgcccttcgaagggctttgtgtctcgactga

SEQ ID NO: 24 - protein, 310 aa, Filobasidium magnum
MTHSPPLDAELSLLRYAPAVPVGWQLGRKLLRMNTLMTRPMEGVMRDDVVIPNLDGTANIRLFICRPQDPTETMPVILWLHGGGMVAGHYKQDSGFMDIWAKRLGAFVVSVDYRLAPEAKAPAALDDCIAAWQWITTQTARGIDTTRMAVGGASAGGGLAASTVQRLVDLGGVKPVFQLLIYPMLDDRTVVRFDPDRRYYMWTPDCNRYGWTSYLGVPPGSAEVPPYASAARRPDLSGLPPTWIGVGSLDLFHDEDMDYARRLRESGVPVEEYVAVGAPHAFDTIYGKAKVTLDFWDSHFNALRRALCLD

SEQ ID NO: 25 - DNA, 933 bp, Filobasidium magnum
  source 1..933: mol_type = other DNA
atgacccactcgccgccactggatgccgaactgagcttgctgcgctacgcccctgccgttccggtgggttggcagctgggtcgcaaactgctgcgtatgaacaccttgatgacccgtccgatggaaggtgtcatgcgcgacgatgtggttattccgaatctggacggcacggctaacatccgtctgtttatctgtcgtccgcaagacccgaccgagactatgccggttatcctgtggctgcacggtggcggcatggtcgcaggccactacaaacaagacagcggtttcatggacatttgggcgaagcgcctgggtgcgtttgttgttagcgttgattatcgcctggcgcctgaggctaaggcaccggcagcgctcgatgactgcatcgcggcgtggcagtggattaccacccagaccgcgcgtggtattgacaccactcgtatggcagtgggtggtgcgagcgcgggtggcggtctggcggcaagcacggttcagcgtcttgtcgatctgggcggtgtgaaaccggtctttcaactgctgatctatccgatgctggacgatcgtaccgtggtgcgcttcgacccggatcgtcgttattacatgtggacgccggactgcaacagatacggctggaccagctacctgggcgtgccaccgggtagcgcagaggtcccgccgtatgcctccgcggctcgtcgtccggatctgtccggcctgccgccgacgtggatcggtgtcggctctctggatctgttccatgacgaagatatggattacgcacgtcgtttgcgcgagagcggtgtgccggtcgaagagtatgttgctgtgggtgccccgcatgcgttcgacacgatttacggcaaggccaaagttacgctggacttttgggatagccacttcaatgcgctgcgccgtgcgttgtgtttagactaa

SEQ ID NO: 26 - DNA, 933 bp, Filobasidium magnum
  source 1..933: mol_type = other DNA
atgactcactctccaccattggacgctgaattgtctttgttgagatacgctccagctgttccagttggttggcaattgggtagaaagttgttgagaatgaacactttgatgactagaccaatggaaggtgttatgagagacgacgttgttatcccaaacttggacggtactgctaacatcagattgttcatctgtagaccacaagacccaactgaaactatgccagttatcttgtggttgcacggtggtggtatggttgctggtcactacaagcaagactctggtttcatggacatctgggctaagagattgggtgctttcgttgtttctgttgactacagattggctccagaagctaaggctccagctgctttggacgactgtatcgctgcttggcaatggatcactactcaaactgctagaggtatcgacactactagaatggctgttggtggtgcttctgctggtggtggtttggctgcttctactgttcaaagattggttgacttgggtggtgttaagccagttttccaattgttgatctacccaatgttggacgacagaactgttgttagattcgacccagacagaagatactacatgtggactccagactgtaacagatacggttggacttcttacttgggtgttccaccaggttctgctgaagttccaccatacgcttctgctgctagaagaccagacttgtctggtttgccaccaacttggatcggtgttggttctttggacttgttccacgacgaagacatggactacgctagaagattgagagaatctggtgttccagttgaagaatacgttgctgttggtgctccacacgctttcgacactatctacggtaaggctaaggttactttggacttctgggactctcacttcaacgctttgagaagagctttgtgtttggactaa

SEQ ID NO: 27 - DNA, 1038 bp, Papiliotrema laurentii
  source 1..1038: mol_type = other DNA
  CDS 1..1038
atgccttccaatctcccccgaccagcatatgacccggaaatagagccattcctctctatggtcccattaccaccaacaatcaatgcggatatcatgagagaactgcgtaaagcgcctctactcagtcaagcgcctgacctcgacgcattactttccggcaaaccaataactcaccgcgaagtcagcattccaggtctcaattcttcagatccacaaatcacgttgtcgatattctccagtacattgacgagcggtccaaaaccatgtatttatttcgttcatggtggcggtatgatcatcggatgtcgattcgtgggtattgaggattatcttcagtacgtcgagcagaatgacgctgtcgtcgtggctgtggaatatcgtcttgcgccggaaaatccagatccagcgcctgtcaatgattgttacgctggacttctatggaccgcagcaaatgctgcagaactgggcattgatctggagagactgttgatctgtggcgcttctgccggtggtggtctttctgctggagtggctttgatggcgcgagacaagaaaggtccgaaactggtaggacaattgttatgttatccgatgctcgacgataggaatgattccctttcaagtcagcagtacgtcgatgaaggtgtttggagtcgtggtagcaatgcattcgggtggaagcaattgcttggagacagggcaggcaaagaaggtgtcagcatctatgctgcaccggcgagggcaactgatttgagcggactgccgaacactttcatcgacgttggcagcgctgaagtcttcagggatgaggacatcgcttatgcctcgaggttatgggctgtcggtgtccaagcagaacttcatgtgtggccgggtggatatcatgctgcggaaaacatggcaccggggactgactactctaacaaggtgaaagctgcccgcttggcatggatgaagagagtcttcatgaaagccccaaagtcgacgacagagtcgttacctgctccaacagtggatgaagctgttggcacaatatga

SEQ ID NO: 28 - protein, 345 aa, Papiliotrema laurentii
MPSNLPRPAYDPEIEPFLSMVPLPPTINADIMRELRKAPLLSQAPDLDALLSGKPITHREVSIPGLNSSDPQITLSIFSSTLTSGPKPCIYFVHGGGMIIGCRFVGIEDYLQYVEQNDAVVVAVEYRLAPENPDPAPVNDCYAGLLWTAANAAELGIDLERLLICGASAGGGLSAGVALMARDKKGPKLVGQLLCYPMLDDRNDSLSSQQYVDEGVWSRGSNAFGWKQLLGDRAGKEGVSIYAAPARATDLSGLPNTFIDVGSAEVFRDEDIAYASRLWAVGVQAELHVWPGGYHAAENMAPGTDYSNKVKAARLAWMKRVFMKAPKSTTESLPAPTVDEAVGTI

SEQ ID NO: 29 - DNA, 1038 bp, Papiliotrema laurentii
  source 1..1038: mol_type = other DNA
atgccaagcaacttgccgcgcccagcctacgatccggaaattgagccttttctgtctatggtcccgctgccgccgaccatcaacgcggacattatgcgtgagctgcgtaaagccccgctgctgagccaggcaccggacctcgacgcactgctgagcggcaagccgatcactcaccgtgaagtcagcattccgggtctgaacagcagcgacccgcaaatcaccctgagcattttctccagcacgttgaccagcggtccgaaaccgtgcatctattttgtgcacggtggcggtatgattattggttgtcgcttcgtcggcattgaagattatctgcaatatgttgagcaaaatgacgcggtggttgtggcggttgagtatcgtctggcccctgaaaatccggacccggcaccggttaatgattgctacgcgggtctgctgtggaccgcagcgaacgcagcggagctgggtatcgatttggaacgcctgctgatctgtggcgcgagcgctggcggtggtctgagcgcgggtgtggcgctgatggctcgcgacaaaaagggtccaaaactggtcggtcagctgttgtgctacccgatgctggacgatcgtaacgacagcttgagctctcaacagtacgtcgatgagggtgtttggagccgtggcagcaatgctttcggctggaaacagctgctgggcgatcgtgcgggtaaggaaggcgtgtcgatctatgccgctccggcacgcgcaaccgatctgtctggcctgccgaacacgttcatcgatgtcggtagcgctgaggtgtttcgtgacgaagatatcgcgtacgcctcacgtctgtgggccgtcggtgtgcaggccgagctgcatgtttggccgggtggctaccatgcagccgagaatatggcgcctggcaccgactactccaataaagtgaaggcagcgcgcctggcgtggatgaagcgtgtgtttatgaaagcgccgaagtccacgaccgagagcctgccggcaccgaccgttgacgaagcggttggtacgatttaa

SEQ ID NO: 30 - DNA, 1038 bp, Bensingtonia ciliata
  source 1..1038: mol_type = other DNA
  CDS 1..1038
atgccttccaatctccctcgaccagcatatgacccggaaatagagccattcctctctatggtcccattaccaccaacaatcaatgcggatatcatgagagaactgcgtaaagcacctctactcagtcaagcgcctgacctcgacgcattactttccggcagaccgataactcaccgcgaagtcagcattccaggtctcaattcccaggatccacaaatcacgttgtcaatattctccagtacattgacgagcggtccaaaaccatgtatttatttcgttcatggtggcggtatgatcatcggatgtcgattcgtgggtattgaggattatcttcaatacgtcgagcagaacgacgctgtcgttgtggctgtggaatatcgtcttgctccggaaaacccggacccagcgcctgttaatgattgttacgccggacttttatggaccgcagcgaatgctgcagagctaggcatcgatctggagagactgttgatctgtggcgcttctgccggtggtggtctttctgctggagtggcattgatggcacgagacaagaaaggtccaaaattggtaggacaattgttatgctatccaatgctcgacgataggaatgattcactctcaagtcagcagtacgtggatgaaggtgtttggagtcgtggtagcaatgcatttggctggaagcaattgcttggagacagggcgggcaaagagggagtcagtatttatgctgcgccggcaagagcaactgatttgagcggactgccgaacactttcatcgacgttggcagcgctgaggtcttcagggatgaggacatcgcttatgcctcgaggttatgggctgtcggtgtccaagcagaacttcatgtgtggcccggtggatatcatgctgcggagaacatggcaccggggactgactactctaagaaggtgaaagctgcgcgcttggcatggatgaagagagtcttcctgaaagccccaaagccgacgactgagtcgttgcctgctccaacagtggatgaagctgttggcacaatatga

SEQ ID NO: 31 - protein, 345 aa, Bensingtonia ciliata
MPSNLPRPAYDPEIEPFLSMVPLPPTINADIMRELRKAPLLSQAPDLDALLSGRPITHREVSIPGLNSQDPQITLSIFSSTLTSGPKPCIYFVHGGGMIIGCRFVGIEDYLQYVEQNDAVVVAVEYRLAPENPDPAPVNDCYAGLLWTAANAAELGIDLERLLICGASAGGGLSAGVALMARDKKGPKLVGQLLCYPMLDDRNDSLSSQQYVDEGVWSRGSNAFGWKQLLGDRAGKEGVSIYAAPARATDLSGLPNTFIDVGSAEVFRDEDIAYASRLWAVGVQAELHVWPGGYHAAENMAPGTDYSKKVKAARLAWMKRVFLKAPKPTTESLPAPTVDEAVGTI

SEQ ID NO: 32 - DNA, 1038 bp, Bensingtonia ciliata
  source 1..1038: mol_type = other DNA
atgccatcgaatctgccgcgtccagcctacgaccctgaaattgaacctttcttgagcatggtgccgctgccgccgacgattaacgctgatatcatgcgtgagctgcgcaaggcaccgctgctgagccaagcgccggacctggatgcgctgttgagcggtcgcccgatcacccaccgcgaagtcagcatcccgggtctgaactctcaggacccgcagatcaccttgtcaatctttagcagcaccttgacttccggtccgaagccgtgcatttattttgtccacggtggtggcatgattatcggctgtcgtttcgttggtattgaagattacttacaatatgtggaacaaaatgatgcagtggttgtggcagtggagtaccgcctggcgcctgagaacccggacccagcgccggtgaacgactgctacgcgggtctgttgtggacggcagctaacgcagcagagctgggtatcgatctggagcgcctgctgatctgcggtgcgagcgcgggtggcggcctgtccgctggcgttgcgctgatggcccgtgacaaaaagggtccgaaactggttggccagctgctgtgttatccgatgctggacgaccgtaatgacagcctgagcagccagcaatacgtggatgagggcgtctggagccgtggtagcaatgcgttcggttggaagcaactgctgggcgatcgtgccggcaaagagggcgttagcatctatgcggcaccggcgcgtgccacggatctgtctggtctgccgaacaccttcattgacgttggtagcgctgaagtttttcgcgatgaagatattgcgtacgcgagccgtctgtgggcagtcggcgtccaggcagagctccatgtctggccgggtggctatcatgcggccgagaatatggcaccgggtacggactacagcaaaaaagttaaagctgcgcgtctggcctggatgaagcgtgttttcctgaaagcgccgaagccgaccaccgagtccctgccggcaccgaccgtggatgaagccgtgggcaccatttaa

SEQ ID NO: 33 - DNA, 474 bp, Rhodococcus erythropolis
  source 1..474: mol_type = other DNA
  CDS 1..474
atgaatctcaacgaagcccgaactgctttcgcccggctccgtgcagcggaaaatggtttatcaccagcagaactcgacgaagtgtgggccgcgctggaaaccgtcgccgctgaagaaatcctcggtgagtggaaaggtgacgacttcgccaccggtcatcgtctgcacgaaaagctgtccgcgagccgctggtacggcaagactttcaattccgtcgaggatgccaagccgttgatctgccgagacgaagacggaaatctctattccgacgtcaagagcggcaatggcgaggcaagtctgtggaacatcgagtttcgtggtgaagtgaccgcgaccatggtctacgacggcgcgccgattttcgaccacttcaagaaagtcgacgattcgacgctcatgggcatcatgaacggaaagtcggcgttggtcctcgacggcgggcagcactactacttcctgctcgagcgagcgtga

SEQ ID NO: 34 - protein, 157 aa, Rhodococcus erythropolis
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFRGEVTATMVYDGAPIFDHFKKVDDSTLMGIMNGKSALVLDGGQHYYFLLERA

SEQ ID NO: 35 - DNA, 474 bp, Rhodococcus erythropolis
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 36 - DNA, 474 bp, Rhodococcus erythropolis
  source 1..474: mol_type = other DNA
atgaacttggacgaagctagaactgctttcgctagattgagagctgctgaatctggtgtttctccagctgaattggacgaagtttgggctgctttggaaactgttgctgctgaagaaatcttgggtgaatggaagggtgacgacttcgctactggtcacagattgcacgaaaagttgttcgcttctagatggtacggtaagactttcaactctgttgaagacgctaagccattgatctgtagagacgaagacggtaacttgtactctgacgttaagtctggtaacggtgaagcttctttgtggaacatcgaattcagaggtgaagttactgctactatggtttacgacggtgctccaatcttcgaccacttcaagaaggttgacgactctactttgatgggtatcatgaacggtaagtctgctttggttttggacggtggtcaacactactacttcttgttggaaagagcttaa

SEQ ID NO: 37 - DNA, 474 bp, Rhodococcus rhodochrous
  source 1..474: mol_type = other DNA
  CDS 1..474
atgaatctcgacgaagcccgaactgctttcgcccggctccgtgctgcggaaagtggtgtatcaccagcagaactcgacgaagtgtgggccgcgctggaaaccgtcgccgccgaagaaatcctcggcgagtggaagggtgacgacttcgccaccggtcaccgtcttcacgaaaagctgttcgcgagccgttggtacggcaagaccttcaactcggtcgaggacgccaagccgttgatctgccgagacgaagacggcaacctctactccgacgtcaagagcggcaatggcgaggcaagtctgtggaacatcgagtttcgtggcgaagtcacggcgacgatggtctacgacggcgcgccgatcttcgaccacttcaagaaggtcgacgattcgacgctcatgggcatcatgaacggaaaatcggcgttggttctcgacggcggacagcactactacttcctgctcgagcgagcgtga

SEQ ID NO: 38 - protein, 157 aa, Rhodococcus rhodochrous
MNLDEARTAFARLRAAESGVSPAELDEVWAALETVAAEEILGEWKGDDFATGHRLHEKLFASRWYGKTFNSVEDAKPLICRDEDGNLYSDVKSGNGEASLWNIEFRGEVTATMVYDGAPIFDHFKKVDDSTLMGIMNGKSALVLDGGQHYYFLLERA

SEQ ID NO: 39 - DNA, 474 bp, Rhodococcus rhodochrous
  source 1..474: mol_type = other DNA
atgaatctggacgaagcccgtactgctttcgcccgtctgcgcgctgctgaatctggtgttagcccggcagagctggacgaagtgtgggcagcgctggaaaccgttgcggcggaagaaattctgggtgagtggaagggcgatgacttcgcaacgggccatcgcttgcacgagaaattgttcgcgagccgctggtatggtaagacctttaacagcgtcgaagatgcgaaaccgctgatctgccgtgatgaagatggcaacctgtacagcgacgtcaagagcggtaatggtgaggccagcctgtggaatatcgagtttcgtggcgaagtgaccgcgacgatggtgtacgacggtgcaccgatttttgatcatttcaaaaaagtcgatgacagcaccctgatgggcatcatgaacggtaagtccgcgctggttctggacggtggccagcactattactttctgctggagcgtgcgtaa

SEQ ID NO: 40 - DNA, 474 bp, Rhodococcus rhodochrous
  source 1..474: mol_type = other DNA
atgaacttgaacgaagctagaactgctttcgctagattgagagctgctgaaaacggtttgtctccagctgaattggacgaagtttgggctgctttggaaactgttgctgctgaagaaatcttgggtgaatggaagggtgacgacttcgctactggtcacagattgcacgaaaagttgtctgcttctagatggtacggtaagactttcaactctgttgaagacgctaagccattgatctgtagagacgaagacggtaacttgtactctgacgttaagtctggtaacggtgaagcttctttgtggaacatcgaattcagaggtgaagttactgctactatggtttacgacggtgctccaatcttcgaccacttcaagaaggttgacgactctactttgatgggtatcatgaacggtaagtctgctttggttttggacggtggtcaacactactacttcttgttggaaagagcttaa

SEQ ID NO: 41 - DNA, 453 bp, Penicillium digitatum
  source 1..453: mol_type = other DNA
  CDS 1..453
atgtccacaagcaccccacaggatcagtttgctgccctagttgcaaaaaacagcaagttgaatgaaaccgacatcgaggctgtttataacaagctttcagctcttcccgtcgatttcctccgtggagaatggaagggtggaagcttcgacaccggccacccaggccacacccagcttttggctatgaactgggttggaaagacgttccacgataccgagcgcgtcgaccctattgttgtgttaaaggatggaaagcgtgtatgcgatgagaactggggccatgctatcgtccgtgaggttcgtttccgtggtattgtgtcaaccgctatgatctatgacaagcaccctatcattgatcacttccgctatgttaatgagaacctcgttgctggcgccatggacactagctccttcggtgacgttggtacctactacttctacctatacaaatag

SEQ ID NO: 42 - protein, 150 aa, Penicillium digitatum
MSTSTPQDQFAALVAKNSKLNETDIEAVYNKLSALPVDFLRGEWKGGSFDTGHPGHTQLLAMNWVGKTFHDTERVDPIVVLKDGKRVCDENWGHAIVREVRFRGIVSTAMIYDKHPIIDHFRYVNENLVAGAMDTSSFGDVGTYYFYLYK

SEQ ID NO: 43 - DNA, 453 bp, Penicillium digitatum
  source 1..453: mol_type = other DNA
atgtccactagcaccccacaagatcaatttgccgcactggttgccaaaaactctaaactgaatgaaaccgacattgaagctgtctataacaagttgagcgcgttgccggtggatttcctgcgtggcgagtggaagggcggcagcttcgacaccggtcacccgggtcacacgcagctgctggcaatgaattgggtcggtaagacctttcatgataccgagcgtgtggacccgatcgtcgttctgaaggacggtaaacgtgtgtgcgacgagaattggggtcacgcgatcgttcgcgaagttcgcttccgtggtatcgtgagcaccgcgatgatctatgataaacacccgattattgatcatttccgctatgttaacgaaaacctggtcgcgggtgcgatggatacgtcgagctttggcgacgtgggcacgtactacttttacctgtacaaataa

SEQ ID NO: 44 - DNA, 453 bp, Penicillium digitatum
  source 1..453: mol_type = other DNA
atgtctacttctactccacaagaccaattcgctgctttggttgctaagaactctaagttgaacgaaactgacatcgaagctgtttacaacaagttgtctgctttgccagttgacttcttgagaggtgaatggaagggtggttctttcgacactggtcacccaggtcacactcaattgttggctatgaactgggttggtaagactttccacgacactgaaagagttgacccaatcgttgttttgaaggacggtaagagagtttgtgacgaaaactggggtcacgctatcgttagagaagttagattcagaggtatcgtttctactgctatgatctacgacaagcacccaatcatcgaccacttcagatacgttaacgaaaacttggttgctggtgctatggacacttcttctttcggtgacgttggtacttactacttctacttgtacaagtaa

SEQ ID NO: 45 - DNA, 453 bp, Penicillium italicum
  source 1..453: mol_type = other DNA
  CDS 1..453
atgtcggccagtgaccccaaggaccagtttgctgccctagttgccaaggacggcaagttgaatgaagacgaaatcgaggctgtttacaacaagcttcctgctcttcccctcgatttcctccgtggagaatggaagggtggaagcttcgacaccggtcaccctggtcacacccaactcttggcaatgaaatgggttgggaagacattccattccaccgaacgggttgaccctattgttgtgttaaaggatgaaaagcgtgtatgcaatgaggactggggccatgcagtcctccgtgagattcgtttccgtggtattgtgtcatctgctatgatctatgacaagcaccctatcatcgaccacttccgctatgtcaacgacaagctcattgctggcgccatggacactagcagcttcggtgacgttggcacctactacttctacctgtgcaaatag

SEQ ID NO: 46 - protein, 150 aa, Penicillium italicum
MSASDPKDQFAALVAKDGKLNEDEIEAVYNKLPALPLDFLRGEWKGGSFDTGHPGHTQLLAMKWVGKTFHSTERVDPIVVLKDEKRVCNEDWGHAVLREIRFRGIVSSAMIYDKHPIIDHFRYVNDKLIAGAMDTSSFGDVGTYYFYLCK

SEQ ID NO: 47 - DNA, 453 bp, Penicillium italicum
  source 1..453: mol_type = other DNA
atgagcgcttcggacccaaaagatcaattcgcagcattggtggcaaaggacggtaaactgaacgaagatgaaatcgaagccgtctataacaagctgcctgcgctgccgctggacttcttgcgtggtgagtggaagggcggcagctttgataccggtcatccgggccacactcagctgctggcgatgaaatgggtgggtaaaacctttcacagcaccgagcgcgtggacccgatcgtcgttctgaaagatgagaagcgtgtctgtaatgaagattggggtcacgccgtgctgcgcgagattcgttttcgcggtatcgtttctagcgcgatgatttatgacaagcatccgattattgaccacttccgttacgttaatgacaagctgatcgcgggtgcgatggatacgtccagctttggcgacgttggcacgtactatttctacctgtgcaaataa

SEQ ID NO: 48 - DNA, 468 bp, Aspergillus wentii
  source 1..468: mol_type = other DNA
  CDS 1..468
atgagctgttgcaccgccgaggaccaggccaaacggctcttcgaagcgaccagccccgtccaaccatcagcagtcgaagaactcttcaaccaactccaaccgataaagccctcattcctgattggcgaatgggacggaaatagcctggacaccggccatcccggtctcaagctgctccaggcgatgcggtgggcgggtaagacatttcgatccgtggatgacgccgatccgattgtgacgctggacgatgctggcaatcgcatctggaaagaggagtacggtaatgctgtggtacgagaaatggcgtttcgcggagtcgtttcggcggcgatgatctacgacaccaagcccatcatggaccattttcgatacgtggacgaaaagacagtgctgggtgtgatggaaacccccaagcaggctggaagcggaaccttttatttctatctgcagcgtcgtgcttctgtctaa

SEQ ID NO: 49 - protein, 155 aa, Aspergillus wentii
MSCCTAEDQAKRLFEATSPVQPSAVEELFNQLQPIKPSFLIGEWDGNSLDTGHPGLKLLQAMRWAGKTFRSVDDADPIVTLDDAGNRIWKEEYGNAVVREMAFRGVVSAAMIYDTKPIMDHFRYVDEKTVLGVMETPKQAGSGTFYFYLQRRASV

SEQ ID NO: 50 - DNA, 468 bp, Aspergillus wentii
  source 1..468: mol_type = other DNA
atgtcgtgttgcaccgccgaagatcaagccaaacgtctgttcgaagccactagcccggttcaaccgagcgcggtcgaagaactgttcaatcagctgcaaccgattaagccttccttcctgatcggtgagtgggatggcaacagcctggataccggtcatccgggcttgaagctgctgcaggcaatgcgctgggcgggtaagacctttcgttctgtggatgacgctgacccaattgttaccctggacgacgcgggtaatcgtatttggaaagaggaatacggtaacgcagtggttcgcgagatggcgtttcgtggtgtggtcagcgcggcaatgatctatgacacgaagccgatcatggatcactttcgctatgttgacgagaaaacggtcctgggcgtgatggaaacgccgaaacaggctggtagcggcaccttctacttttacttgcagcgtcgtgcgagcgtctaa

SEQ ID NO: 51 - DNA, 468 bp, Aspergillus wentii
  source 1..468: mol_type = other DNA
atgtcttgttgtactgctgaagaccaagctaagagattgttcgaagctacttctccagttcaaccatctgctgttgaagaattgttcaaccaattgcaaccaatcaagccatctttcttgatcggtgaatgggacggtaactctttggacactggtcacccaggtttgaagttgttgcaagctatgagatgggctggtaagactttcagatctgttgacgacgctgacccaatcgttactttggacgacgctggtaacagaatctggaaggaagaatacggtaacgctgttgttagagaaatggctttcagaggtgttgtttctgctgctatgatctacgacactaagccaatcatggaccacttcagatacgttgacgaaaagactgttttgggtgttatggaaactccaaagcaagctggttctggtactttctacttctacttgcaaagaagagcttctgtttaa

SEQ ID NO: 52 - DNA, 477 bp, Rhodococcus hoagii
  source 1..477: mol_type = other DNA
  CDS 1..477
atgaacgtacgagacgaggtcgccgcgctgcgggcgcgcaccgaccggatcgatccgcgggaactcgattcgatctgggaccgcttggccccgtgtcggcccgtggatctgatcgggtaccgctggaggggtttcgacttcgacaccggacatcgcacgagtgggctcctccgtcgagctcattggtacggcaaggcattcgccagcgagtccgacgtgcagcctctgctgtgccgcagcgaggacggacagctgttctccgacatcggaaccgggcacggcgaggccagcctgtgggaggtcgtgttccgcggggaggtgaccgcgacgatggtctacgacggcatgccggtgttcgaccacttcaagaaggtcgacgacgacaccgtcatcggcgtcatgaacggcaagggcacgttggtgttcgacggcggcgaacacttctggttcgggctggagcgagacgtcgcactctga

SEQ ID NO: 53 - protein, 158 aa, Rhodococcus hoagii
MNVRDEVAALRARTDRIDPRELDSIWDRLAPCRPVDLIGYRWRGFDFDTGHRTSGLLRRAHWYGKAFASESDVQPLLCRSEDGQLFSDIGTGHGEASLWEVVFRGEVTATMVYDGMPVFDHFKKVDDDTVIGVMNGKGTLVFDGGEHFWFGLERDVAL

SEQ ID NO: 54 - DNA, 477 bp, Rhodococcus hoagii
  source 1..477: mol_type = other DNA
atgaatgttcgtgatgaagttgcagctctgcgtgcccgtactgatagaatcgacccgcgtgagctggatagcatttgggaccgtctggcaccatgtcgtccggtggacctgatcggttaccgttggcgcggtttcgatttcgacaccggtcaccgtacctccggtctgttgcgtcgcgcgcattggtatggtaaggcctttgcgagcgagagcgacgtgcaaccgttgctgtgccgctctgaggacggccagctgtttagcgatattggcaccggtcacggtgaggcgagcctgtgggaagttgtctttcgcggcgaagtgaccgcgacgatggtttacgacggtatgccggtgttcgaccacttcaaaaaagttgatgacgacacggtgatcggtgtcatgaacggcaagggcacgctggtctttgatggtggcgagcatttctggtttggcctggaacgcgatgtcgcgctgtaa

SEQ ID NO: 55 - DNA, 477 bp, Rhodococcus hoagii
  source 1..477: mol_type = other DNA
  CDS 1..477
atgaacgtacgagacgaggtcgccgcgctgcgggcgcgcaccgaccggatcgatccgcgggaactcgattcgatctgggaccgcttggccccgtgtcggcccgtggatctgatcgggtaccgctggaggggtttcgacttcgacaccggacatcgcacgagtgggctcctccgtcgagctcattggtacggcaaggcattcgccagcgagtccgacgtgcagcctctgctgtgccgcagcgacgacggacagctgttctccgacatcggaaccgggcacggcgaggccagcctgtgggaggtcgtgttccgcggggaggtgaccgcgacgatggtctacgacggcatgccggtgttcgaccacttcaagaaggtcgacgacgacaccgtcatcggcgtcatgaacggcaagggcacgttggtgttcgacggcggcgaacacttctggttcgggctggagcgagacgtcgcactctga

SEQ ID NO: 56 - protein, 158 aa, Rhodococcus hoagii
MNVRDEVAALRARTDRIDPRELDSIWDRLAPCRPVDLIGYRWRGFDFDTGHRTSGLLRRAHWYGKAFASESDVQPLLCRSDDGQLFSDIGTGHGEASLWEVVFRGEVTATMVYDGMPVFDHFKKVDDDTVIGVMNGKGTLVFDGGEHFWFGLERDVAL

SEQ ID NO: 57 - DNA, 477 bp, Rhodococcus hoagii
  source 1..477: mol_type = other DNA
atgaatgttcgtgatgaagttgcagctctgcgtgcccgtactgatagaatcgacccgcgtgagctggatagcatttgggaccgtctggcaccatgtcgtccggtggacctgatcggttaccgttggcgcggtttcgatttcgacaccggtcaccgtacctccggtctgttgcgtcgcgcgcattggtatggtaaggcctttgcgagcgagagcgacgtgcaaccgttgctgtgccgctctgatgacggccagctgtttagcgatattggcaccggtcacggtgaggcgagcctgtgggaagttgtctttcgcggcgaagtgaccgcgacgatggtttacgacggtatgccggtgttcgaccacttcaaaaaagttgatgacgacacggtgatcggtgtcatgaacggcaagggcacgctggtctttgatggtggcgagcatttctggtttggcctggaacgcgatgtcgcgctgtaa

SEQ ID NO: 58 - DNA, 477 bp, Rhodococcus hoagii
  source 1..477: mol_type = other DNA
  CDS 1..477
atgaacgtacgagacgaggtcgccgcgctgcgggcgcgcaccgaccggatcgatccgcgggaactcgattcgatctgggaccgcttggccccgtgtcggcccgtggatctgatcgggtaccgctggcggggtttcgacttcgacaccggacatcgcacgagtgggctcctccgtcgagcgcattggtacggcaaggcattcgccagcgagtccgacgtgcagcctctgctgtgccgcagcgaggacggacagctgttctccgacatcggaaccgggcacggcgaggccagcctgtgggaggtcgtgttccgcggggaggtgaccgcgacgatggtctacgacggcatgccggtgtccgaccacttcaagaaggtcgacgacgacaccgtcatcggcgtcatgaacggcaagggcacgttggtgttcgacggcggcgaacacttctggttcgggctggagcgagacgtcgcactctga

SEQ ID NO: 59 - protein, 158 aa, Rhodococcus hoagii
MNVRDEVAALRARTDRIDPRELDSIWDRLAPCRPVDLIGYRWRGFDFDTGHRTSGLLRRAHWYGKAFASESDVQPLLCRSEDGQLFSDIGTGHGEASLWEVVFRGEVTATMVYDGMPVSDHFKKVDDDTVIGVMNGKGTLVFDGGEHFWFGLERDVAL

SEQ ID NO: 60 - DNA, 477 bp, Rhodococcus hoagii
  source 1..477: mol_type = other DNA
atgaatgttcgtgatgaagttgcagctctgcgtgcccgtactgatagaatcgacccgcgtgagctggatagcatttgggaccgtctggcaccatgtcgtccggtggacctgatcggttaccgttggcgcggtttcgatttcgacaccggtcaccgtacctccggtctgttgcgtcgcgcgcattggtatggtaaggcctttgcgagcgagagcgacgtgcaaccgttgctgtgccgctctgaggacggccagctgtttagcgatattggcaccggtcacggtgaggcgagcctgtgggaagttgtctttcgcggcgaagtgaccgcgacgatggtttacgacggtatgccggtgagcgaccacttcaaaaaagttgatgacgacacggtgatcggtgtcatgaacggcaagggcacgctggtctttgatggtggcgagcatttctggtttggcctggaacgcgatgtcgcgctgtaa

SEQ ID NO: 61 - DNA, 585 bp, Cupriavidus necator
  source 1..585: mol_type = other DNA
  CDS 1..585
atgctgacagaaatgctgcggaatcgagtctctacaactgcggcggtactggccgctttcgatgaacttgatccattatcgagcgattcgctagttggctgctggagtggttttgtgatcgctaccgggcaccccatggacggtcttctgagcgctgtcggctggtacgggaaaatgttccaaagcgtggatgaggcatatccgctgatcatccggtccccggacgccagtacgcttttttcgatcgatcccagccctttgccacttataggctgcgcgaagttatctcccacggatatggtgtcgcgtttttcaacactttccccgttggccctgagcacaaccgtctctcacggtcggctgcgtatggtcgagtatcgcggaaaggtcacaggaactctgatctacgaccagcagccgatactcgatcatttcgtgatgattgattcgcaaacggtacttggaattatggattttaaagagttcccgcagccaggcgcgtttgtgctgcagcgcgatgacgacagtgccgtcagcgttgatcgcggcgactggtcccaactggcggcgcaacggctcgggtga

SEQ ID NO: 62 - protein, 194 aa, Cupriavidus necator
MLTEMLRNRVSTTAAVLAAFDELDPLSSDSLVGCWSGFVIATGHPMDGLLSAVGWYGKMFQSVDEAYPLIIRSPDASTLFSIDPSPLPLIGCAKLSPTDMVSRFSTLSPLALSTTVSHGRLRMVEYRGKVTGTLIYDQQPILDHFVMIDSQTVLGIMDFKEFPQPGAFVLQRDDDSAVSVDRGDWSQLAAQRLG

SEQ ID NO: 63 - DNA, 585 bp, Cupriavidus necator
  source 1..585: mol_type = other DNA
atgttgactgaaatgctgcgtaaccgtgtgtctaccactgccgctgtcctggccgcttttgacgagctggacccgctgtcatccgacagcctggttggctgctggagcggtttcgttatcgcgacgggtcaccctatggatggtctgctgagcgcggtgggctggtacggtaaaatgttccagagcgttgatgaagcatacccgctgatcatccgctccccggacgcgagcacgctgtttagcattgatccgtccccgctgccgctgattggttgtgcgaagctgtcgccaaccgatatggtgagccgcttcagcaccttaagcccgctggcgctgagcaccaccgtatctcacggtcgtctgcgtatggttgagtatcgtggtaaggttaccggcacgctcatctatgaccaacagccgattttggatcatttcgtcatgattgacagccaaacggtgctgggcatcatggatttcaaagaatttccgcagccgggtgcgtttgtcttgcagcgtgacgacgatagcgcagtcagcgtggatcgcggcgactggagccaactggcagcccaacgcctgggctaa

SEQ ID NO: 64 - DNA, 585 bp, Cupriavidus necator
  source 1..585: mol_type = other DNA
atgttgactgaaatgttgagaaacagagtttctactactgctgctgttttggctgctttcgacgaattggacccattgtcttctgactctttggttggttgttggtctggtttcgttatcgctactggtcacccaatggacggtttgttgtctgctgttggttggtacggtaagatgttccaatctgttgacgaagcttacccattgatcatcagatctccagacgcttctactttgttctctatcgacccatctccattgccattgatcggttgtgctaagttgtctccaactgacatggtttctagattctctactttgtctccattggctttgtctactactgtttctcacggtagattgagaatggttgaatacagaggtaaggttactggtactttgatctacgaccaacaaccaatcttggaccacttcgttatgatcgactctcaaactgttttgggtatcatggacttcaaggaattcccacaaccaggtgctttcgttttgcaaagagacgacgactctgctgtttctgttgacagaggtgactggtctcaattggctgctcaaagattgggttaa

SEQ ID NO: 65 - DNA, 447 bp, Penicillium italicum
  source 1..447: mol_type = other DNA
  CDS 1..447
atgacaatccaattcccaatcatgtcattcgactgtttccagccaagcccagccaagaaattcgtctctctcaccaaacaccctcgggtgactggtgggaaaatcaacaccgtctttcctgagctcaagcctcttcagccagacgacctaatcggcgaatgggacggatatattcttgtcacgggccacccctttgaagaagaactggacacgctgaattggttcggaaatacattttattccaccgacgacgtggcaccgctgactgttgcgcggaacggggtgcgggtgcccttcgaggattgggggcgtgcatctctacgtgaaatcaaatatcaaggagtcgtctctgcggctttggtctatgataaacgaccaatgatggtctattatcgagccgtgaaacataacatggtggctgggggtattgagagtaaagagtggtag

SEQ ID NO: 66 - protein, 148 aa, Penicillium italicum
MTIQFPIMSFDCFQPSPAKKFVSLTKHPRVTGGKINTVFPELKPLQPDDLIGEWDGYILVTGHPFEEELDTLNWFGNTFYSTDDVAPLTVARNGVRVPFEDWGRASLREIKYQGVVSAALVYDKRPMMVYYRAVKHNMVAGGIESKEW

SEQ ID NO: 67 - DNA, 447 bp, Penicillium italicum
  source 1..447: mol_type = other DNA
atgaccattcaatttcctatcatgtcttttgattgttttcagccgagcccagcgaagaaattcgtgagcttgacgaaacatccgcgtgttaccggtggcaagatcaatacggttttcccggaactgaaaccgctgcaaccggacgacctgatcggtgagtgggacggttacattctggtgacgggccacccgttcgaagaagaactggataccttgaactggttcggcaatactttctatagcaccgacgatgtcgctccgctgaccgtcgcccgcaacggtgtgcgtgttccgtttgaggattggggtcgtgcgtccctgcgtgagatcaagtaccagggtgtggttagcgcagcgctggtctacgacaaacgcccgatgatggtgtattatcgcgcagttaagcacaacatggtcgcgggtggcattgagagcaaagagtggtaa

SEQ ID NO: 68 - DNA, 501 bp, Ralstonia insidiosa
  source 1..501: mol_type = other DNA
  CDS 1..501
atgaacacgaagcagaaattcgatcaactcaagagcacggaacgcctgaatgacgaaatcctgttggagttcttcgacacccttccccccgtttctacggacgaagcgctgggtcgctggaaaggcggtgacttcaatacggggcattggggcaacctcgctctgaaagcaaggaagtggtacggaaagtggtatcgcagcaagctggatgcggtaccgcttatctgttacgacgaccaaggccgcctatattccagcaaggccatgaagggcgaagcgtcgctttgggatgtggcgttccgcggaaaggtctcgaccaccatgatctacgacggcgtgccgatcttcgatcatttgcgcaaggtcgacgagaacacgctgttcggcatcatggatggcaaatcgtttgaggggtcccccgacatcatcgaccgcggcaagtactactttttctacctcgagagggtagacagcttcccggccgaatatctggaaggctga

SEQ ID NO: 69 - protein, 166 aa, Ralstonia insidiosa
MNTKQKFDQLKSTERLNDEILLEFFDTLPPVSTDEALGRWKGGDFNTGHWGNLALKARKWYGKWYRSKLDAVPLICYDDQGRLYSSKAMKGEASLWDVAFRGKVSTTMIYDGVPIFDHLRKVDENTLFGIMDGKSFEGSPDIIDRGKYYFFYLERVDSFPAEYLEG

SEQ ID NO: 70 - DNA, 501 bp, Ralstonia insidiosa
  source 1..501: mol_type = other DNA
atgaacaccaagcaaaagtttgaccagctgaagtccaccgagcgcctgaatgatgaaatcctgttggaatttttcgataccctgcctccggtgagcaccgatgaagcgctgggccgttggaagggtggcgacttcaatacgggtcattggggtaacctggccctgaaagcgcgtaaatggtacggcaaatggtatcgcagcaaactggacgcagttccactgatttgctatgacgatcagggccgtctgtactctagcaaggctatgaaaggtgaggcgagcctgtgggatgttgcgtttcgtggtaaagtgagcacgactatgatctacgacggtgtcccgattttcgaccacttgcgtaaagtcgatgagaacacgctgtttggtatcatggatggtaagtcgttcgagggtagcccggacattatcgaccgtggcaagtactatttcttttatctggagcgcgttgacagcttcccggcagagtacctggaaggctaa

SEQ ID NO: 71 - DNA, 447 bp, Cryptococcus gattii
  source 1..447: mol_type = other DNA
  CDS 1..447
atgtcccctcaggaacagtatattgctctcgtccaggccggcggcaagtcggacccatccaccattgaagctcttttccaagcgcttccgccggtcaagcccactcagctgctaggcgactggaatcacggcggatttttcgacacaggccatccggttaacgagcaactcaaagagattaaatggattggaaagtcatttaagtccgtcgaagatgttgatcctgtgattattgaccaggatggtaagccaactagctggaggaagtgggggtcagccagcctgcgagagatggtgtatgaaggcactgtatcaacgtcgatgatatatgatgaccgaccaatcatcgatcacttccgctacgtagatgacgactttatggcggggataatggaagggaaggctctgggggaggcggggaagttttatttctatttgagaagatag

SEQ ID NO: 72 - protein, 148 aa, Cryptococcus gattii
MSPQEQYIALVQAGGKSDPSTIEALFQALPPVKPTQLLGDWNHGGFFDTGHPVNEQLKEIKWIGKSFKSVEDVDPVIIDQDGKPTSWRKWGSASLREMVYEGTVSTSMIYDDRPIIDHFRYVDDDFMAGIMEGKALGEAGKFYFYLRR

SEQ ID NO: 73 - DNA, 447 bp, Cryptococcus gattii
  source 1..447: mol_type = other DNA
atgagcccacaagaacaatacattgcattagtccaggccggtggtaagagcgatcctagcacgatcgaagcgctgtttcaggcattgccgccggttaaaccgacccagctgctgggcgattggaatcacggtggcttctttgacacgggccatccggtgaacgaacaactgaaagaaatcaagtggattggcaaatccttcaaatcggtcgaagatgttgatccggtgatcatcgaccaggacggtaagccgactagctggcgtaagtggggttctgcgagcctgcgtgagatggtttatgagggcaccgtgagcaccagcatgatttatgacgaccgcccgatcattgatcactttcgttacgtcgatgacgacttcatggctggtattatggaaggcaaggcactgggtgaggccggtaaattctacttttatctgcgccgttaa

SEQ ID NO: 74 - DNA, 459 bp, Grosmannia clavigera
  source 1..459: mol_type = other DNA
  CDS 1..459
atgacagctgtacagcgatttaacgcactcaccaaagcagaagggcttctcaaggagtctgagcttgcacaaattttcgacgagctccctcctgtttctccagaagctatgacaggcaagtggaatggaggcagctttgacagtggccatcctgtccacaagctgcttcaaacttttaaatgggcagggaaagaattccgctccgttgacgatatcgacccgattgtgatcttcgacgaaaatggggagcgaaagtggctatccgagtatggacatgcaagactgcgtgaagttaagtttcggggagttgtatctgccgccttggtatacgacaaagttgccattatcgactcgtttcgtcgggtttcggacaacgtgctgatgggaactatggacgccagggactggccggatgctggcatctactacttttacatcaccaagtttgaagaattgtga

SEQ ID NO: 75 - protein, 152 aa, Grosmannia clavigera
MTAVQRFNALTKAEGLLKESELAQIFDELPPVSPEAMTGKWNGGSFDSGHPVHKLLQTFKWAGKEFRSVDDIDPIVIFDENGERKWLSEYGHARLREVKFRGVVSAALVYDKVAIIDSFRRVSDNVLMGTMDARDWPDAGIYYFYITKFEEL

SEQ ID NO: 76 - DNA, 459 bp, Grosmannia clavigera
  source 1..459: mol_type = other DNA
atgactgctgttcaacgttttaacgcattgaccaaagccgagggtttgctgaaagaatctgagctggcacagattttcgacgaactgccgccggttagcccagaggccatgaccggtaagtggaatggtggcagctttgattccggccatccggtgcacaagctgctgcagacgttcaaatgggcgggtaaagaatttcgtagcgttgacgacattgacccgatcgtgatctttgatgagaatggcgagcgcaagtggctgagcgagtatggtcacgcacgcctgcgtgaagtgaagttccgtggtgtcgtcagcgcggctctggtctatgacaaagtcgcgatcattgacagcttccgccgtgttagcgataacgtgctgatgggtacgatggatgcgcgtgattggccggatgcgggcatttactacttctacatcaccaagtttgaagaactgtaa

SEQ ID NO: 77 - DNA, 642 bp, Oidiodendron maius
  source 1..642: mol_type = other DNA
  CDS 1..642
atggcttctactttatatgaagctagagttattttggcacttaaagctattcaaaacagcaacaatcttagcttacgagctgcagcaaagctgtatgatgtacagccaacaaccctatattaccgacaagctggccgacctgcacgacatgatattccacctaactctcgcaagcttacggatctagaagaggagacgattgttcgcccgacggaacagtttattgccctagctcaggcccagggccggcttgatgccacattgattgacgcggtgtttaacaagtttggcccagtcaagccagagctgatgctaggcaagtggagtggtgggattttagacaccggccatcctatgggagatacactgaaggagatacgatgggtgggcaagaatttcacctccactgaacacgtggacccggttattatcgacaagaacggccaaagggccagctgggggaagtggggccttgctaccctacgtgaggtcttgtatcgagatgttgtctcgacggcgatgatctacgatgaccgcccggtctttgactatttccgtttcgctaatgatgatatggttgctggtatcatggaagggaaggagttgggagggagacttttctatttctacctgaagagatag

SEQ ID NO: 78 - protein, 213 aa, Oidiodendron maius
MASTLYEARVILALKAIQNSNNLSLRAAAKLYDVQPTTLYYRQAGRPARHDIPPNSRKLTDLEEETIVRPTEQFIALAQAQGRLDATLIDAVFNKFGPVKPELMLGKWSGGILDTGHPMGDTLKEIRWVGKNFTSTEHVDPVIIDKNGQRASWGKWGLATLREVLYRDVVSTAMIYDDRPVFDYFRFANDDMVAGIMEGKELGGRLFYFYLKR

SEQ ID NO: 79 - DNA, 642 bp, Oidiodendron maius
  source 1..642: mol_type = other DNA
atggcaagcactttgtatgaagctcgcgtgattctggcgctgaaagcgattcaaaatagcaacaatctgagcttgcgtgcagccgcgaagctctatgatgtccagccgaccacgctgtactatcgtcaggccggtcgtccagctcgccacgacatcccgccgaactcccgtaagctgaccgatctggaagaggaaacgatcgttcgcccgaccgagcaattcatcgcgttagcacaagcacagggccgtctggatgcgaccctgattgatgcagttttcaataagtttggtccggtgaagcctgagctgatgctgggtaagtggagcggtggcattctggacacgggtcacccgatgggcgataccctgaaagaaatccgttgggtgggtaaaaatttcaccagcaccgaacatgttgatccggtcatcattgacaaaaacggtcagcgcgcttcttggggcaagtggggtctggccaccttgcgtgaagttctgtaccgcgacgtcgtcagcacggcgatgatttacgatgaccgtccggtgtttgactattttcgtttcgcgaacgacgacatggttgcgggtatcatggaaggcaaagaactgggtggccgtctgttttacttctacctgaaacgctaa

SEQ ID NO: 80 - DNA, 447 bp, Thermomonospora curvata
  source 1..447: mol_type = other DNA
  CDS 1..447
atggatgcggaacagcgccttgccaagatcatcgcgtccggcgacgagtgcgaccgggccaccgtggaggaactgtacgaccggctggcccccgtgccggtggacttcatgctcggcacctggcggggcggcatcttcgaccggggcgacgcgctggcggggatgctgctggggatgaactggtacggcaagcggttcatcgaccgcgaccacgtcgagccgctgctgtgccgctcccccgacggctcgatctactcctacgagaagctcgggctggcccggctgcgcgaggtcgccctgcgcggcacggtctcggcggccatgatctacgacaagcagcccatcatcgaccacttccggcgggtcaacgacgacatggtggtcggcgccatggacgccaagggccagcccgacatcctctacttccacctcacccgggaacgctga

SEQ ID NO: 81 - protein, 148 aa, Thermomonospora curvata
MDAEQRLAKIIASGDECDRATVEELYDRLAPVPVDFMLGTWRGGIFDRGDALAGMLLGMNWYGKRFIDRDHVEPLLCRSPDGSIYSYEKLGLARLREVALRGTVSAAMIYDKQPIIDHFRRVNDDMVVGAMDAKGQPDILYFHLTRER

SEQ ID NO: 82 - DNA, 447 bp, Thermomonospora curvata
  source 1..447: mol_type = other DNA
atggatgcggaacaaagactggctaaaattattgcatctggtgatgagtgtgatcgtgcaaccgtggaagaactgtatgaccgtttggcccctgtcccggttgacttcatgctgggtacgtggcgtggtggcatcttcgatcgtggtgatgcgctggcgggtatgctgctgggtatgaattggtatggcaagcgctttatcgaccgcgaccacgtcgagccactgctgtgccgtagcccggatggctccatctacagctacgagaaactgggtctggcccgtttgcgcgaagtggcactgcgtggcaccgttagcgcggctatgatttatgacaaacagccgattatcgaccatttccgtcgcgtgaacgacgacatggttgtcggcgcgatggatgcgaagggtcagccggacatcctgtactttcacctgacccgcgagcgttaa

SEQ ID NO: 83 - DNA, 492 bp, Pseudomonas litoralis
  source 1..492: mol_type = other DNA
  CDS 1..492
atgactgcaacactggccgccctcagcctgaccaccctgcttgccgggcccagtctggccgcagatacggaacagcaatggctggagatgatcgccagcggtgaagcctattcggcggacaccctggtgcctctgttcaaacaactcgaaccggtggataccgacttcatggtcggcacatggaagggcggcaagttcgacggcggcgccgagccggacccgatcaactggtacggcaaacgtttcacctcgaccaccgatgtcgagccgttattggtaaacgatgccgagggcgaggtgatcacccacgaccggctcggcgccgcacagatgcgccaggtggtgttcgatgggaaggtatcggccgcgttgatctacgacagccagccgatcatggattacctccgcaaggtcaacgaggatgtggtcatcggcctgggcgacatcaagggcaagcctaccgatttctttttctatctggtacgcgattaa

SEQ ID NO: 84 - protein, 163 aa, Pseudomonas litoralis
MTATLAALSLTTLLAGPSLAADTEQQWLEMIASGEAYSADTLVPLFKQLEPVDTDFMVGTWKGGKFDGGAEPDPINWYGKRFTSTTDVEPLLVNDAEGEVITHDRLGAAQMRQVVFDGKVSAALIYDSQPIMDYLRKVNEDVVIGLGDIKGKPTDFFFYLVRD

SEQ ID NO: 85 - DNA, 492 bp, Pseudomonas litoralis
  source 1..492: mol_type = other DNA
atgactgcgactttggctgctctgagcttgacgaccctgttggctggcccatctttggctgcggacaccgagcagcaatggctggaaatgattgcaagcggcgaggcgtatagcgcggacaccctggtgccgctgttcaagcaactggagcctgtcgatacggacttcatggtcggcacgtggaagggcggcaaatttgatggtggtgccgaaccggacccgattaactggtacggtaagcgttttaccagcacgaccgatgtggagccgctgctggtgaatgacgccgagggtgaagttatcacccacgatcgtctgggtgcggcacagatgcgccaagttgtttttgatggcaaagtctccgcagcgctgatctacgacagccagccgattatggactatctgcgcaaagtgaacgaagatgttgtcatcggtctgggtgacatcaagggtaaaccgaccgactttttcttctacctggttcgtgattaa

SEQ ID NO: 86 - DNA, 498 bp, Pseudomonas protegens
  source 1..498: mol_type = other DNA
  CDS 1..498
atgaatacgaaagaaaagtttgaacagcttaagagcacgcaaggtcttaatgatgaagtactgttggacttctttgactcgctttctccagtcacaatcgatggcgcgttgggccgttggcaaggtggtgacttcaagacaggacactggggcaatgacgcacttaccggaatgaagtggtacggaaagtggtaccggagcaagttggatgccgttcccctagtctgctacgacgaacagggccgactattttccaacaagatcatgaaaggtgaggcctctctctgggaggtggcgtttcgtggcaaggtttcgactacgatgatctacgatggcgttccgatttatgatcacttgcgcaaggtcgatgacaacacccttttcgggatcatggatggtaagtcctttgaggggcagctccccgacatcatcgacaatggcaagtactacttcttctacctcgaaagggtcgatggcttccctgtcgagttcgtctag

SEQ ID NO: 87 - protein, 165 aa, Pseudomonas protegens
MNTKEKFEQLKSTQGLNDEVLLDFFDSLSPVTIDGALGRWQGGDFKTGHWGNDALTGMKWYGKWYRSKLDAVPLVCYDEQGRLFSNKIMKGEASLWEVAFRGKVSTTMIYDGVPIYDHLRKVDDNTLFGIMDGKSFEGQLPDIIDNGKYYFFYLERVDGFPVEFV

SEQ ID NO: 88 - DNA, 498 bp, Pseudomonas protegens
  source 1..498: mol_type = other DNA
atgaacacgaaagaaaagtttgaacagttgaaaagcacccaaggtctgaacgatgaagttttgctggatttcttcgatagcctgagcccagtgaccattgacggtgcactgggccgttggcagggtggcgacttcaagaccggtcactggggcaacgacgcgctgactggcatgaaatggtacggtaaatggtatcgcagcaaactggatgctgtgccgctggtgtgctacgacgaacagggtcgtctgttttccaataagatcatgaaaggtgaggccagcctgtgggaagtcgcgttccgcggtaaggttagcacgacgatgatttatgatggtgtgccgatctatgaccatctgcgtaaagttgatgacaataccctgtttggcatcatggatggcaagtcttttgagggtcaactgccggacatcattgacaatggcaagtactacttcttctacctggagcgtgttgacggttttccggtcgagttcgtctaa

SEQ ID NO: 89 - protein, 157 aa, synthetic construct
  REGION 1..157: note = variant
MNLNEARTAFARLRAAENGLSPAELDEVWAALETVAAEEILGEAKGDDFATGHRLHEKLSASRWYGKTFNSVEDAKPLICRDEDGNLYSDVKSGNGEASLWNIEFRGEVTATMVYDGAPIFDHFKKVDDSTLMGIMNGKSALVLDGGQHYYFLLERA

SEQ ID NO: 90 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgaggcaaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 91 - protein, 157 aa, synthetic construct
  REGION 1..157: note = variant
MNLNEARTAFARLRAAENGLSPAELDEVWAALETVAAEEILGEWKGDDFAAGHRLHEKLSASRWYGKTFNSVEDAKPLICRDEDGNLYSDVKSGNGEASLWNIEFRGEVTATMVYDGAPIFDHFKKVDDSTLMGIMNGKSALVLDGGQHYYFLLERA

SEQ ID NO: 92 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcggccggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 93 - protein, 157 aa, synthetic construct
  REGION 1..157: note = variant
MNLNEARTAFARLRAAENGLSPAELDEVWAALETVAAEEILGEWKGDDFATGARLHEKLSASRWYGKTFNSVEDAKPLICRDEDGNLYSDVKSGNGEASLWNIEFRGEVTATMVYDGAPIFDHFKKVDDSTLMGIMNGKSALVLDGGQHYYFLLERA

SEQ ID NO: 94 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtgcacgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 95 - protein, 157 aa, synthetic construct
  REGION 1..157: note = variant
MNLNEARTAFARLRAAENGLSPAELDEVWAALETVAAEEILGEWKGDDFATGHRLHEKASASRWYGKTFNSVEDAKPLICRDEDGNLYSDVKSGNGEASLWNIEFRGEVTATMVYDGAPIFDHFKKVDDSTLMGIMNGKSALVLDGGQHYYFLLERA

SEQ ID NO: 96 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaagcatcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 97 - protein, 157 aa, synthetic construct
  REGION 1..157: note = variant
MNLNEARTAFARLRAAENGLSPAELDEVWAALETVAAEEILGEWKGDDFATGHRLHEKLSASRAYGKTFNSVEDAKPLICRDEDGNLYSDVKSGNGEASLWNIEFRGEVTATMVYDGAPIFDHFKKVDDSTLMGIMNGKSALVLDGGQHYYFLLERA

SEQ ID NO: 98 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgcgcctatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 99 - protein, 157 aa, synthetic construct
  REGION 1..157: note = variant
MNLNEARTAFARLRAAENGLSPAELDEVWAALETVAAEEILGEWKGDDFATGHRLHEKLSASRWYGATFNSVEDAKPLICRDEDGNLYSDVKSGNGEASLWNIEFRGEVTATMVYDGAPIFDHFKKVDDSTLMGIMNGKSALVLDGGQHYYFLLERA

SEQ ID NO: 100 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtgcaaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 101 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNAVEDAKPLICRDEDGNLYSDVKSGNGEASLWNIEFRGEVTATMVYDGAPIFDHFKKVDDSTLMGIMNGKSALVLDGGQHYYFLLERA

SEQ ID NO: 102 - DNA, 474 bp, synthetic construct
  misc_feature 1..474: note = E. Coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaacgcagttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 103 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFAGEVTATMVYDGAPIFDHFKKVDDSTLMGIMNGKSALVLDGGQHYYFLLERA

SEQ ID NO: 104 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttgcaggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 105 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFRGEVTATMVADGAPIFDHFKKVDDSTLMGIMNGKSALVLDGGQHYYFLLERA

SEQ ID NO: 106 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggttgccgatggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 107 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFRGEVTATMVYAGAPIFDHFKKVDDSTLMGIMNGKSALVLDGGQHYYFLLERA

SEQ ID NO: 108 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgccggtgccccgatcttcgaccatttcaaaaaagttgacgacagcaccctgatgggcattatgaatggcaaaagcgcgttggtgttggacggtggccagcattactatttcctgctggagcgtgcgtaa

SEQ ID NO: 109 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFRGEVTATMVYDGAPIFAHFKKVDDSTLMGIMNGKSALVLDGGQHYYFLLERA

SEQ ID NO: 110 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgcacatttcaaaaaagttgacgacagcaccctgatgggcattatgaatggcaaaagcgcgttggtgttggacggtggccagcattactatttcctgctggagcgtgcgtaa

SEQ ID NO: 111 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFRGEVTATMVYDGAPIFDHFKKVDDSTLMGIANGKSALVLDGGQHYYFLLERA

SEQ ID NO: 112 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattgcaaatggcaaaagcgcgttggtgttggacggtggccagcattactatttcctgctggagcgtgcgtaa

SEQ ID NO: 113 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFRGEVTATMVYDGAPIFDHFKKVDDSTLMGIMNGASALVLDGGQHYYFLLERA

SEQ ID NO: 114 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcgcaagcgcgttggtgttggacggtggccagcattactatttcctgctggagcgtgcgtaa

SEQ ID NO: 115 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFRGEVTATMVYDGAPIFDHFKKVDDSTLMGIMNGKSALVLDGGQHYYALLERA

SEQ ID NO: 116 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatgcactgctggagcgtgcgtaa

SEQ ID NO: 117 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFRGEVTATMVYDGAPIFDHFKKVDDSTLMGIMNGKSALVLDGGQHYYFLAERA

SEQ ID NO: 118 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctggccgagcgtgcgtaa

SEQ ID NO: 119 - protein, 157 aa, synthetic construct
  REGION 1..157: note = variant
MNLNEARTAFARLRAAENGLSPAELDEVWAALETVAAEEILGEWKGDDFATGHRLHEKLSASRWYGKTFNSVEDAKPLICRDEDGNLYSDVKSGNGEASLWNIEFRGEVTATMVYDGAPIFDHFKKVDDSTLMGIMNGKSALVLDGGQHYYFLLEAA

SEQ ID NO: 120 - DNA, 474 bp, synthetic construct
  misc_feature 1..474: note = E. coli optimized
  source 1..474: mol_type = other DNA
atgaacttaaatgaagcgcgtaccgcatttgcacgtctgagagctgccgagaacggtctgagcccggctgagctggatgaagtgtgggcagcgctggagactgtggcggctgaagaaatcctgggtgagtggaagggtgacgattttgcgacgggtcaccgtctgcacgagaaactgtcggcgagccgctggtatggtaagaccttcaactctgttgaagatgcaaagccgctgatttgccgtgacgaagatggcaatctgtactccgatgtcaagagcggtaacggtgaggccagcctgtggaatatcgagtttcgcggcgaagtcaccgcgacgatggtttacgatggtgccccgatcttcgaccatttcaaaaaagttgacgacagcaccctgatgggcattatgaatggcaaaagcgcgttggtgttggacggtggccagcattactatttcctgctggaggcagcgtaa

SEQ ID NO: 121 - DNA, 60 bp, synthetic construct
  misc_feature 1..60: note = Integration cassette fragment 1
  source 1..60: mol_type = other DNA
gcaggcagctccatttcatgtaggtgatttatccctccgggcggtatttgagactctcgg

SEQ ID NO: 122 - DNA, 60 bp, synthetic construct
  misc_feature 1..60: note = Integration cassette fragment 2
  source 1..60: mol_type = other DNA
actgctgggtactgttcaggcacgataggaaatgcgtccagcgcatacaccagtcttagc

SEQ ID NO: 123 - DNA, 60 bp, synthetic construct
  misc_feature 1..60: note = Integration cassette fragment 3
  source 1..60: mol_type = other DNA
agtcgaccttacagcgcctgggactctacataaacatgcagcgaacatgctttccaacgc

SEQ ID NO: 124 - DNA, 79 bp, synthetic construct
  misc_feature 1..79: note = LEU2 yeast marker_primer 1
  source 1..79: mol_type = other DNA
aggtgcagttcgcgtgcaattataacgtcgtggcaactgttatcagtcgtaccgcgccattcgactacgtcgtaaggcc

SEQ ID NO: 125 - DNA, 80 bp, synthetic construct
  misc_feature 1..80: note = LEU2 yeast marker_primer 2
  source 1..80: mol_type = other DNA
tcgtggtcaaggcgtgcaattctcaacacgagagtgattcttcggcgttgttgctgaccatcgacggtcgaggagaactt

SEQ ID NO: 126 - DNA, 85 bp, synthetic construct
  misc_feature 1..85: note = AmpR E. coli marker_primer 1
  source 1..85: mol_type = other DNA
tggtcagcaacaacgccgaagaatcactctcgtgttgagaattgcacgccttgaccacgacacgttaagggattttggtcatgag

SEQ ID NO: 127 - DNA, 80 bp, synthetic construct
  misc_feature 1..80: note = AmpR E. coli marker_primer 2
  source 1..80: mol_type = other DNA
aacgcgtaccctaagtacggcaccacagtgactatgcagtccgcactttgccaatgccaaaaatgtgcgcggaaccccta

SEQ ID NO: 128 - DNA, 84 bp, synthetic construct
  misc_feature 1..84: note = Yeast origin of replication_primer 1
  source 1..84: mol_type = other DNA
ttggcattggcaaagtgcggactgcatagtcactgtggtgccgtacttagggtacgcgttcctgaacgaagcatctgtgcttca

SEQ ID NO: 129 - DNA, 85 bp, synthetic construct
  misc_feature 1..85: note = Yeast origin of replication_primer 2
  source 1..85: mol_type = other DNA
ccgagatgccaaaggataggtgctatgttgatgactacgacacagaactgcgggtgacataatgatagcattgaaggatgagact

SEQ ID NO: 130 - DNA, 82 bp, synthetic construct
  misc_feature 1..82: note = E. coli replication origin_primer 1
  source 1..82: mol_type = other DNA
atgtcacccgcagttctgtgtcgtagtcatcaacatagcacctatcctttggcatctcggtgagcaaaaggccagcaaaagg

SEQ ID NO: 131 - DNA, 81 bp, synthetic construct
  misc_feature 1..81: note = E. coli replication origin_primer 2
  source 1..81: mol_type = other DNA
ctcagatgtacggtgatcgccaccatgtgacggaagctatcctgacagtgtagcaagtgctgagcgtcagaccccgtagaa

SEQ ID NO: 132 - DNA, 60 bp, synthetic construct
  misc_feature 1..60: note = DNA fragment for S. cerevisiae co-transformation
  source 1..60: mol_type = other DNA
attcctagtgacggccttgggaactcgatacacgatgttcagtagaccgctcacacatgg

SEQ ID NO: 133 - DNA, 1056 bp, Hyphozyma roseonigra
  source 1..1056: mol_type = other DNA
  CDS 1..1056
atgcaattcagcatcggagatgtactcgccattgtagataaaacaatcctcaacccactcgtcgtcagcgcaggacttctgtctctgcactttctcaccaatgacaaatacgcaatcactgcgaatgacggtctattcccttatcaaattagcactccagactcgcatcgaaaagccctttttgcacttggctttggtctacttctcagagccaatcgctacatgagcagaaaagctctgaacaacaacaccgccgcacaattcgactggaatcgtgagatcatcgttgttactggtggatctggtggtatcggtgctcaggccgcgcagaaattggcagaaagaggatcgaaagtgattgttattgatgtgctaccacttacctttgacaagcccaagaatttgtaccactataaatgtgatctcacaaactacaaagagctccaagaagttgcggctaagatcgaaagagaagttggcactccgacttgtgtagttgcgaatgcaggaatatgtcgtggaaagaacatattcgatgctacagaacgagatgttcagcttacctttggagtcaacaatctgggacttctatggacagccaaaacctttctcccatcaatggccaaagcaaatcatggccatttcttgatcatcgcctctcaaaccggccatctagcgaccgcaggagtagtcgactatgcagcgaccaaagcagcagcaatcgccatatatgaaggtctacaaacagagatgaagcacttttataaagcgcctgctgtacgcgtatcttgtatctccccatccgcggtcaagacgaagatgtttgcaggcatcaagactggaggcaatttcttcatgccaatgttgacgcctgatgatcttggagacctgattgcaaagactttgtgggacggtgtggcagtcaatattttgagccctgcggcggcatatatcagcccgcccacgagagctttgccagattggatgagggttggcatgcaggatgctggtgctgagatcatgacggaattgactcctcataagccgttggagtag

SEQ ID NO: 134 - protein, 351 aa, Hyphozyma roseonigra
MQFSIGDVLAIVDKTILNPLVVSAGLLSLHFLTNDKYAITANDGLFPYQISTPDSHRKALFALGFGLLLRANRYMSRKALNNNTAAQFDWNREIIVVTGGSGGIGAQAAQKLAERGSKVIVIDVLPLTFDKPKNLYHYKCDLTNYKELQEVAAKIEREVGTPTCVVANAGICRGKNIFDATERDVQLTFGVNNLGLLWTAKTFLPSMAKANHGHFLIIASQTGHLATAGVVDYAATKAAAIAIYEGLQTEMKHFYKAPAVRVSCISPSAVKTKMFAGIKTGGNFFMPMLTPDDLGDLIAKTLWDGVAVNILSPAAAYISPPTRALPDWMRVGMQDAGAEIMTELTPHKPLE

SEQ ID NO: 135 - DNA, 1056 bp, Hyphozyma roseonigra
  source 1..1056: mol_type = other DNA
atgcaattctctatcggtgacgttttggctatcgttgacaagactatcttgaacccattggttgtttctgctggtttgttgtctttgcacttcttgactaacgacaagtacgctatcactgctaacgacggtttgttcccataccaaatctctactccagactctcacagaaaggctttgttcgctttgggtttcggtttgttgttgagagctaacagatacatgtctagaaaggctttgaacaacaacactgctgctcaattcgactggaacagagaaatcatcgttgttactggtggttctggtggtatcggtgctcaagctgctcaaaagttggctgaaagaggttctaaggttatcgttatcgacgttttgccattgactttcgacaagccaaagaacttgtaccactacaagtgtgacttgactaactacaaggaattgcaagaagttgctgctaagatcgaaagagaagttggtactccaacttgtgttgttgctaacgctggtatctgtagaggtaagaacatcttcgacgctactgaaagagacgttcaattgactttcggtgttaacaacttgggtttgttgtggactgctaagactttcttgccatctatggctaaggctaaccacggtcacttcttgatcatcgcttctcaaactggtcacttggctactgctggtgttgttgactacgctgctactaaggctgctgctatcgctatctacgaaggtttgcaaactgaaatgaagcacttctacaaggctccagctgttagagtttcttgtatctctccatctgctgttaagactaagatgttcgctggtatcaagactggtggtaacttcttcatgccaatgttgactccagacgacttgggtgacttgatcgctaagactttgtgggacggtgttgctgttaacatcttgtctccagctgctgcttacatctctccaccaactagagctttgccagactggatgagagttggtatgcaagacgctggtgctgaaatcatgactgaattgactccacacaagccattggaataa

SEQ ID NO: 136 - DNA, 1023 bp, Hyphozyma roseonigra
  source 1..1023: mol_type = other DNA
  CDS 1..1023
atggcgacgataccgaccacaatgaccgcagcgacaatcgttgaattcaacaagcccatcgtgctaaagaacgacataccagttccagacctaccagagaacaagattcttgttaggatagctgcaacatcattatgctcaagcgacttgatggcgtacaaaggttacatggatttcatgaccaagacgccttactgcggaggacacgagcccgtgggaacggtggtgaaagtcggttcttcggtaaaaggctactcggttggagatcgcgttggcatattgatgttcttcgatacctgtggaacatgcaatgactgcttctcgggtgaacatcgcttttgcagcacaaagaaaatcctaggcttcgcggaaagctggggaggattttcagaatacgcacttgctgatcccatctcgaccatcaagctcccggaagggttgagtttcgatgtagcagcgcctttgttctgcgctgggatcacagcctacagcgcgctgttgaaggtgaagagtcatgccggtcaactcatcaatatcatcggctgtggaggcgtaggacatatggctatattgtatgcgagagctatgggatatcgagttcatgtttacgatatatccgattccaaggtcgaatttgcactctttctcggcgcagatgcagccttcaacactctgacctataccggtccaatagaatcagcatcttctacgttagtcgtaagtggagcaaatgcagcataccagagcgctctaggcatgacgagtaatcatggagtcgtcctgggtattggactaccagcgggaggtgtggtcattgatgtgccagcttggggtacgaaaggcgttacattcgtcccatgcaacacaggctcgaaacaggaactagaagaagcgctagaattggctgtgagaaaggatatcaaaccattacttgacatccgccatatcgacacaattaatgaggcatatcaagatttggcggagggaaagatcaatgggaggattgttttccacttcgagtga

SEQ ID NO: 137 - protein, 340 aa, Hyphozyma roseonigra
MATIPTTMTAATIVEFNKPIVLKNDIPVPDLPENKILVRIAATSLCSSDLMAYKGYMDFMTKTPYCGGHEPVGTVVKVGSSVKGYSVGDRVGILMFFDTCGTCNDCFSGEHRFCSTKKILGFAESWGGFSEYALADPISTIKLPEGLSFDVAAPLFCAGITAYSALLKVKSHAGQLINIIGCGGVGHMAILYARAMGYRVHVYDISDSKVEFALFLGADAAFNTLTYTGPIESASSTLVVSGANAAYQSALGMTSNHGVVLGIGLPAGGVVIDVPAWGTKGVTFVPCNTGSKQELEEALELAVRKDIKPLLDIRHIDTINEAYQDLAEGKINGRIVFHFE

SEQ ID NO: 138 - DNA, 1023 bp, Hyphozyma roseonigra
  source 1..1023: mol_type = other DNA
atggctactatcccaactactatgactgctgctactatcgttgaattcaacaagccaatcgttttgaagaacgacatcccagttccagacttgccagaaaacaagatcttggttagaatcgctgctacttctttgtgttcttctgacttgatggcttacaagggttacatggacttcatgactaagactccatactgtggtggtcacgaaccagttggtactgttgttaaggttggttcttctgttaagggttactctgttggtgacagagttggtatcttgatgttcttcgacacttgtggtacttgtaacgactgtttctctggtgaacacagattctgttctactaagaagatcttgggtttcgctgaatcttggggtggtttctctgaatacgctttggctgacccaatctctactatcaagttgccagaaggtttgtctttcgacgttgctgctccattgttctgtgctggtatcactgcttactctgctttgttgaaggttaagtctcacgctggtcaattgatcaacatcatcggttgtggtggtgttggtcacatggctatcttgtacgctagagctatgggttacagagttcacgtttacgacatctctgactctaaggttgaattcgctttgttcttgggtgctgacgctgctttcaacactttgacttacactggtccaatcgaatctgcttcttctactttggttgtttctggtgctaacgctgcttaccaatctgctttgggtatgacttctaaccacggtgttgttttgggtatcggtttgccagctggtggtgttgttatcgacgttccagcttggggtactaagggtgttactttcgttccatgtaacactggttctaagcaagaattggaagaagctttggaattggctgttagaaaggacatcaagccattgttggacatcagacacatcgacactatcaacgaagcttaccaagacttggctgaaggtaagatcaacggtagaatcgttttccacttcgaataa

SEQ ID NO: 139 - DNA, 1116 bp, Filobasidium magnum
  source 1..1116: mol_type = other DNA
  CDS 1..1116
atgccaacccctatctttggcgcccgagagggtttcactatcgactccgtactgagcatcctggatgcgaccgtacttaacccctggtttaccggcgtgtgcctaatagccgtctgcgcccgagatcgcaccattacgtacccggactggccggcggctctggaccaggtgctccccttcttgtcgcagatgtggagggaaactgtcagaccgacctttggcgaccgcaacgtccttcatctgttgaccactgtgtgtgtcggccttgccatccgaaccaacagacggatgagtcggggagcgaggaacaattgggtgtgggatactagttatgactggaagaaggagatcgtagtggttacgggaggagctgccgggtttggtgcagacatcgtacaacagctagacacgcgtggaatccaggtcgtcgtcttggatgtgggatccctcacctataggccttcgagcagagttcattattacaagtgcgacgtgtcgaacccacaagacgtcgccagcgtggctaaagctatcgtatccaacgtcgggcacccgaccatattggtcaacaacgctggcgtattcaggggtgcgactattctctccacgacaccgcgcgacctcgacatgacctacgacatcaacgtcaaagcgcactatcatctcacgaaggcgttcctcccgaacatgatctccaagaaccatggacatattgtgactgtgtcaagcgcgaccgcatacgctcaagcttgttctggcgtgtcatactgttcctcaaaggccgccatcttgtcatttcacgaaggactgagcgaagagattttgtggatctataaggcgcccaaagtccggacctcggtcatctgccccggacacgtcaatacggccatgtttacaggcattggagccgccgctccctcgttcatggcacctgcacttcatccctcgacagtcgccgagacaatcgtcgatgtattgctctcatgcgagtctcaacacgtcctgatgcccgccgccatgcacatgtcagtcgccggacgagcgctgcccacctggttcttccgggggttgttggcatcgggcaaggataccatgggtagcgttgtccgccgatga

SEQ ID NO: 140 - protein, 371 aa, Filobasidium magnum
MPTPIFGAREGFTIDSVLSILDATVLNPWFTGVCLIAVCARDRTITYPDWPAALDQVLPFLSQMWRETVRPTFGDRNVLHLLTTVCVGLAIRTNRRMSRGARNNWVWDTSYDWKKEIVVVTGGAAGFGADIVQQLDTRGIQVVVLDVGSLTYRPSSRVHYYKCDVSNPQDVASVAKAIVSNVGHPTILVNNAGVFRGATILSTTPRDLDMTYDINVKAHYHLTKAFLPNMISKNHGHIVTVSSATAYAQACSGVSYCSSKAAILSFHEGLSEEILWIYKAPKVRTSVICPGHVNTAMFTGIGAAAPSFMAPALHPSTVAETIVDVLLSCESQHVLMPAAMHMSVAGRALPTWFFRGLLASGKDTMGSVVRR

SEQ ID NO: 141 - DNA, 1116 bp, Filobasidium magnum
  source 1..1116: mol_type = other DNA
atgccaactccaatcttcggtgctagagaaggtttcactatcgactctgttttgtctatcttggacgctactgttttgaacccatggttcactggtgtttgtttgatcgctgtttgtgctagagacagaactatcacttacccagactggccagctgctttggaccaagttttgccattcttgtctcaaatgtggagagaaactgttagaccaactttcggtgacagaaacgttttgcacttgttgactactgtttgtgttggtttggctatcagaactaacagaagaatgtctagaggtgctagaaacaactgggtttgggacacttcttacgactggaagaaggaaatcgttgttgttactggtggtgctgctggtttcggtgctgacatcgttcaacaattggacactagaggtatccaagttgttgttttggacgttggttctttgacttacagaccatcttctagagttcactactacaagtgtgacgtttctaacccacaagacgttgcttctgttgctaaggctatcgtttctaacgttggtcacccaactatcttggttaacaacgctggtgttttcagaggtgctactatcttgtctactactccaagagacttggacatgacttacgacatcaacgttaaggctcactaccacttgactaaggctttcttgccaaacatgatctctaagaaccacggtcacatcgttactgtttcttctgctactgcttacgctcaagcttgttctggtgtttcttactgttcttctaaggctgctatcttgtctttccacgaaggtttgtctgaagaaatcttgtggatctacaaggctccaaaggttagaacttctgttatctgtccaggtcacgttaacactgctatgttcactggtatcggtgctgctgctccatctttcatggctccagctttgcacccatctactgttgctgaaactatcgttgacgttttgttgtcttgtgaatctcaacacgttttgatgccagctgctatgcacatgtctgttgctggtagagctttgccaacttggttcttcagaggtttgttggcttctggtaaggacactatgggttctgttgttagaagataa

SEQ ID NO: 142 - DNA, 1023 bp, Filobasidium magnum
  source 1..1023: mol_type = other DNA
  CDS 1..1023
atggagccaccccagactatgaaggccgccttggtcaccgcatacaacgagcccctgattgtgaaagacgttgctacacccgagccgggccctggacagattctcgttcgggtcaaagctagttcgctttgcatgtcagatatcggaggctatgtcggagcgatgggggaatttatcacgctcccctattgtccaggtcatgaacccgccggagagatcgtcgcccttggcgacaacgtgtccggcttctccgttggggatcgggttacttatatggccgctctagatccttgtatgggctgccgagactgtctccgaggtgccattcgattctgctccaaacgctcgaatctcggcttcagccaccagtacggcgggttctccgagtactctctcgccagcccatactcgatggccaaggtgccggacgaactgtccctcgaggaagctgcgagcatgtcctgtgccggggtgaccgctttcggtgctctcaagctattgagcaagtatcaggctccgggaggcatcatcaatgtcttgggctgcggcggcgttggtcatctggtcatcaagtttgccgtcgcgctcggctacaccgtgcacgctttcgacattaacgatggcaaactcaaactggccgaggagtgcggggcatctaaagccttcctttcaaagggagatcccacccaggcgatgatggcagagagtacgatagtcatctcgggtgtcaacgcggcctatgatttcgctattaaagctactctggccggtggacgtatcattgcgattgggcacccacattcggcgactccgatgcctctcggctcgatgatcatcaacgacatctcgttgatcgtgagcaatcaaggtacaagggtggatctacaagaagccttggatttcgccgctcgatccggtgtcagaccgaacattacaatcaacgaaggtctggacggcatcaatcagggctataaatcggtcatgacaggcgctgtagaaggcagattggtctacaaattctag

SEQ ID NO: 143 - protein, 340 aa, Filobasidium magnum
MEPPQTMKAALVTAYNEPLIVKDVATPEPGPGQILVRVKASSLCMSDIGGYVGAMGEFITLPYCPGHEPAGEIVALGDNVSGFSVGDRVTYMAALDPCMGCRDCLRGAIRFCSKRSNLGFSHQYGGFSEYSLASPYSMAKVPDELSLEEAASMSCAGVTAFGALKLLSKYQAPGGIINVLGCGGVGHLVIKFAVALGYTVHAFDINDGKLKLAEECGASKAFLSKGDPTQAMMAESTIVISGVNAAYDFAIKATLAGGRIIAIGHPHSATPMPLGSMIINDISLIVSNQGTRVDLQEALDFAARSGVRPNITINEGLDGINQGYKSVMTGAVEGRLVYKF

SEQ ID NO: 144 - DNA, 1023 bp, Filobasidium magnum
  source 1..1023: mol_type = other DNA
atggaaccaccacaaactatgaaggctgctttggttactgcttacaacgaaccattgatcgttaaggacgttgctactccagaaccaggtccaggtcaaatcttggttagagttaaggcttcttctttgtgtatgtctgacatcggtggttacgttggtgctatgggtgaattcatcactttgccatactgtccaggtcacgaaccagctggtgaaatcgttgctttgggtgacaacgtttctggtttctctgttggtgacagagttacttacatggctgctttggacccatgtatgggttgtagagactgtttgagaggtgctatcagattctgttctaagagatctaacttgggtttctctcaccaatacggtggtttctctgaatactctttggcttctccatactctatggctaaggttccagacgaattgtctttggaagaagctgcttctatgtcttgtgctggtgttactgctttcggtgctttgaagttgttgtctaagtaccaagctccaggtggtatcatcaacgttttgggttgtggtggtgttggtcacttggttatcaagttcgctgttgctttgggttacactgttcacgctttcgacatcaacgacggtaagttgaagttggctgaagaatgtggtgcttctaaggctttcttgtctaagggtgacccaactcaagctatgatggctgaatctactatcgttatctctggtgttaacgctgcttacgacttcgctatcaaggctactttggctggtggtagaatcatcgctatcggtcacccacactctgctactccaatgccattgggttctatgatcatcaacgacatctctttgatcgtttctaaccaaggtactagagttgacttgcaagaagctttggacttcgctgctagatctggtgttagaccaaacatcactatcaacgaaggtttggacggtatcaaccaaggttacaagtctgttatgactggtgctgttgaaggtagattggtttacaagttctaa

SEQ ID NO: 145 - DNA, 1038 bp, Rhodococcus sp.
  source 1..1038: mol_type = other DNA
  CDS 1..1038
atgaaagccgtccagtacaccgagatcggctccgagccggtcgttgtcgacatccccaccccgacgcccgggccgggtgagatcctgctgaaggtcaccgcggccgggctgtgccactcggacatcttcgtgatggacatgccggcggcgcagtacgcctacggcctgccgctcaccctcggccacgagggtgtcggcaccgtcgccgaactcggcgagggcgtcacgggattcggggtgggggacgccgtcgccgtgtacgggccgtggggctgcggtgcgtgccacgcctgcgcgcgcggccgggagaactactgcacccgcgccgccgacctgggcatcacgccacccggtctcggctcgcccggatcgatggccgagtacatgatcgtcgattcggcgcgccacctcgtcccgatcggagacctcgacccggtcgccgcggcgccgctcaccgacgccggtctgacgccgtaccacgcgatctcccgggtcctgccgctgctggggccgggctcgacggccgtcgtcatcggtgtcggcgggctcggccacgtcggcatccagatcctgcgcgccgtcagcgcggcccgtgtgatcgccgtcgacctcgacgacgaccgtctcgccctcgcccgcgaggtcggcgccgacgcggcggtgaagtcgggcgccggtgcggcggacgcgatccgggaactgaccggcggccagggcgcgacggcggtgttcgacttcgtcggcgcccagtcgacgatcgacacggcgcagcaggtggtcgcggtcgacgggcacatctcggtcgtgggcatccacgccggcgcacacgccaaggtcgggttcttcatgatcccgttcggcgcctccgtcgtgaccccgtactggggcacccggtcggaactgatggaggtcgtcgcgctggcccgcgccggccggctggacatccacaccgagacgttcaccctcgacgaggggccggcggcgtaccggcggctgcgcgagggcagcatccgcggccgcggcgtggtggttccctga

SEQ ID NO: 146 - protein, 345 aa, Rhodococcus sp.
MKAVQYTEIGSEPVVVDIPTPTPGPGEILLKVTAAGLCHSDIFVMDMPAAQYAYGLPLTLGHEGVGTVAELGEGVTGFGVGDAVAVYGPWGCGACHACARGRENYCTRAADLGITPPGLGSPGSMAEYMIVDSARHLVPIGDLDPVAAAPLTDAGLTPYHAISRVLPLLGPGSTAVVIGVGGLGHVGIQILRAVSAARVIAVDLDDDRLALAREVGADAAVKSGAGAADAIRELTGGQGATAVFDFVGAQSTIDTAQQVVAVDGHISVVGIHAGAHAKVGFFMIPFGASVVTPYWGTRSELMEVVALARAGRLDIHTETFTLDEGPAAYRRLREGSIRGRGVVVP

SEQ ID NO: 147 - DNA, 1038 bp, Rhodococcus sp.
  source 1..1038: mol_type = other DNA
atgaaagcagtgcaatatacggaaattggctcggaacctgttgtggtggacatcccgaccccgaccccgggtcctggtgaaatcctgctgaaagttaccgctgccggcctgtgccacagcgacatcttcgtgatggacatgccggctgcccagtacgcttacggtctgccactgacgctgggtcacgaaggcgtgggtacggtcgccgaactgggcgagggtgtcaccggtttcggtgtcggtgatgccgtggcagtgtacggtccgtggggttgcggtgcgtgccacgcgtgtgcgcgcggccgcgagaattactgtacgcgtgcagcggacctgggtattaccccgccgggcctgggcagcccgggtagcatggcagagtacatgatcgttgatagcgcacgtcatctggtgccgattggcgatttggacccggtcgcggcagccccgctgactgacgcgggcttgaccccgtatcatgcaatctcccgtgtactgccactgctcggtccgggcagcaccgctgtggttatcggcgtcggtggcctgggtcatgttggcatccagattctgcgtgcagtcagcgcggcacgcgtgatcgcggttgatctggacgacgatcgcctggccctggcgcgtgaggtcggtgcggatgctgcggttaagtctggtgccggtgcagcggatgcgattcgtgagctgacgggtggccagggtgcgaccgccgtgtttgatttcgttggcgcgcagagcaccattgatacggcgcaacaagtcgttgcggtcgatggtcacatttccgttgtgggtatccacgcgggtgcacatgccaaggtcggctttttcatgatcccgtttggtgctagcgttgttaccccgtattggggcacgcgcagcgagctgatggaagtcgtggctttggcgcgtgcgggtcgtctggacattcacaccgagactttcaccttggacgaaggcccggcagcgtatcgtcgtctgcgcgagggtagcattcgtggccgtggtgttgttgtcccgtaa

SEQ ID NO: 148 - DNA, 1113 bp, Rhodococcus rhodochrous
  source 1..1113: mol_type = other DNA
  CDS 1..1113
atgaagaccaaagctgctgtactgctcgagcccggaaagcctttcgagatcatggaactcgacctcgacggcccgggtgtgggtgaggtactgatcaagtacaccgctgccggactgtgccattcggatctgcacctgaccgacggtgatctcccgccgcgttacccgatcgtcggcggacacgaaggctcgggcatcatcgaagaggtcggcccaggcgtcacgaaggtcaagccgggcgaccatgtcgtgtgtagcttcatccccaactgcggcacctgtcgctactgctcgaccggtcgccagaacctctgcgacatgggtgccaccatcctcgaaggctcgatgcccgacggttccttccgtttccacggcaacggaatggatttcggcggaatgtgcatgttgggaacgttctccgagcgcgccaccatttctcagcactcggtagtcaagatcgacgactggcttcccttggagacagcggtggtcgtcggctgcggcgtgccttcgggttggggaacggcagtaaatgccggtaaccttcgcgccggtgacaccgctgtgatctacggcatcggtggtctcggcatcaacgccgtccagggcgccgtttcggccggctgcaagtacgtcgttgtggtcgatccggttgctctcaagcgtgagaccgcactgaagttcggtgcaacccatgcctttgcagacgccgagagcgctgctgccaaggtcaacgagctgacgtggggacagggtgccgacgctgcgctcatccttgtcggcaccgtcgacgaggacgtggtcagtgcagcgacggcagtgatcggcaagggtggcaccgtggtgatcacgggactcgccgaccccgccaagctgaccgttcacgtgtcgggtaccgacctgacgctgaatcagaagacgatcaagggcacgttgttcgggtccatgaatccgcagtacgacatcgtgcgactgctgcgtctctacgatgccggtcagctcaagctcgacgaactgatcaccaacacctacagcctcgaagacgtcaacaagggctaccaggatctacgtgacggcaagaacatccgtggcgtgatcattcacgacaagtaa

SEQ ID NO: 149 - protein, 370 aa, Rhodococcus rhodochrous
MKTKAAVLLEPGKPFEIMELDLDGPGVGEVLIKYTAAGLCHSDLHLTDGDLPPRYPIVGGHEGSGIIEEVGPGVTKVKPGDHVVCSFIPNCGTCRYCSTGRQNLCDMGATILEGSMPDGSFRFHGNGMDFGGMCMLGTFSERATISQHSVVKIDDWLPLETAVVVGCGVPSGWGTAVNAGNLRAGDTAVIYGIGGLGINAVQGAVSAGCKYVVVVDPVALKRETALKFGATHAFADAESAAAKVNELTWGQGADAALILVGTVDEDVVSAATAVIGKGGTVVITGLADPAKLTVHVSGTDLTLNQKTIKGTLFGSMNPQYDIVRLLRLYDAGQLKLDELITNTYSLEDVNKGYQDLRDGKNIRGVIIHDK

SEQ ID NO: 150 - DNA, 1113 bp, Rhodococcus rhodochrous
  source 1..1113: mol_type = other DNA
atgaaaacgaaagccgcagtgttgttggagccgggcaaaccatttgagatcatggaactggatctggacggtccgggtgtcggtgaagtgctgatcaagtacaccgcagcgggcttgtgccactctgatctgcacctgaccgacggcgacttgccgccacgttacccgattgtgggtggccatgagggtagcggtatcattgaagaggttggtccgggcgttaccaaggtcaaaccgggtgatcacgtcgtgtgctctttcatcccgaattgtggtacgtgccgctattgtagcacgggtcgtcagaacctgtgcgacatgggtgccaccattttagagggctccatgcctgatggctccttccgttttcacggcaacggtatggactttggtggcatgtgcatgctgggtacgttcagcgaacgcgcgaccatcagccaacatagcgtcgttaagatcgatgactggctcccgctggaaaccgcagttgttgttggttgtggtgttccgagcggttggggtactgcggtcaatgccggtaatctgcgtgctggtgacaccgcggtcatttatggtattggcggcctgggtatcaacgctgtgcagggcgcagttagcgcgggctgcaaatacgtcgttgtggttgacccggttgcgctgaaacgtgagactgcgctgaaatttggcgcaacccacgcgttcgcagacgcggagagcgcagctgcgaaagtgaacgaactgacctggggtcagggtgcggatgcggcactgatcttggtcggcaccgtggacgaagatgtcgtgagcgcggcgactgctgttatcggtaagggtggcaccgttgtgatcaccggtctggccgatccggcaaagctgaccgttcatgtcagcggtacggacctgaccctgaatcagaaaaccattaagggcacgctgttcggttcgatgaacccgcagtacgacattgtgcgcctgctgcgtctgtacgatgcgggtcaactgaaactggacgaacttattacgaatacgtatagcctggaagatgtgaacaaaggctaccaagatctgcgtgatggtaagaatattcgtggtgtcattatccacgacaagtga

SEQ ID NO: 151 - DNA, 1041 bp, Rhodococcus rhodochrous
  source 1..1041: mol_type = other DNA
  CDS 1..1041
atgaaggcagcccagctcatggggcccgggctcctggaaatcaacgacgtgccggtcccggagatcggcccgtcggaactactgattcgggtgggcgcagcgggaatctgccactccgatctccatctcctgcactttccgtacaagatgcgcgaagaaccgctgacaatcggccacgaaattgccggaacgatcgaagccgtcgggagtggcgtcgacggccgttccgtcggagagcgtggcgtcgtctacctctgttggtcatgtggacagtgccgagaatgcatgagcggcaacgagaatatgtgcctcgccgctggacgcaccgcgatgccgccctgccccggactcggccctgagggcgggatggccgagtacgtcaagatcccggctcgctcattcgtacccatcggagacctcgacttcctgcaggccgcacctctcgccgatgcggcactgacgagctaccacgccattcgcggtgcccgcgaacatctccagcccggtgccaccgccgtcgtgatcggcgtcggcggactcggtcacgttgcagtacagatacttcgcgcgatcagtgccgtgcgcatcatcgccgtcgatgtcggacaggatcaactcgatctcgccaaacgttgcggcgccgacatcacgctcgaatcgggaccggacaccgcgcagcacatcctcgacctcacatcggccagaggcgcagaagtcatcttcgacttcgtcggtatcgacgcaactgcacagatgtctgttcaagcggttgcgccgaacggcgcgtatcgcatggtaggtctcggaggcggaaaccccggaatcactgccgaagctgccggcggaccaggctggccatggggcgcatcgatccggaagtcctacggcggcaccagaaacgacctcgtcgattccatcgccctggcacaggccggtctggtaacggtagaagtagcccgcttcgacctcgctgatgcccgcgacgcactcgaccgtctcgaacacggcaaggtcaccggacgcgcagtgctcgtaccctga

SEQ ID NO: 152 - protein, 346 aa, Rhodococcus rhodochrous
MKAAQLMGPGLLEINDVPVPEIGPSELLIRVGAAGICHSDLHLLHFPYKMREEPLTIGHEIAGTIEAVGSGVDGRSVGERGVVYLCWSCGQCRECMSGNENMCLAAGRTAMPPCPGLGPEGGMAEYVKIPARSFVPIGDLDFLQAAPLADAALTSYHAIRGAREHLQPGATAVVIGVGGLGHVAVQILRAISAVRIIAVDVGQDQLDLAKRCGADITLESGPDTAQHILDLTSARGAEVIFDFVGIDATAQMSVQAVAPNGAYRMVGLGGGNPGITAEAAGGPGWPWGASIRKSYGGTRNDLVDSIALAQAGLVTVEVARFDLADARDALDRLEHGKVTGRAVLVP

SEQ ID NO: 153 - DNA, 1041 bp, Rhodococcus rhodochrous
  source 1..1041: mol_type = other DNA
atgaaagctgcacaactgatgggtccgggtctgttggaaattaatgatgttccagtcccagaaattggtccgagcgagctgctgatccgtgttggcgctgccggcatttgccacagcgatctgcatctgctgcacttcccgtacaagatgcgtgaggaaccgttaaccattggtcacgaaatcgcgggcacgatcgaagccgttggtagcggtgtggatggccgcagcgttggtgagcgtggcgtggtttacctgtgctggtcctgtggtcagtgccgcgagtgcatgtccggcaatgaaaacatgtgtctggcggctggtcgtaccgcaatgccgccatgtccgggtttgggtcctgagggtggcatggccgaatatgtcaagatcccggcgcgtagcttcgtgccgattggcgatctggactttctgcaggcagcgcctttggcggacgcagcactgaccagctaccacgcgatccgtggtgcccgcgaacacttgcagccgggtgcaaccgcagtggtcattggtgtcggcggcttgggtcatgtggcagtgcaaatcctgcgcgcgatttctgcggtccgtatcattgcggttgatgtgggccaggaccaactggacctggcgaagcgttgtggcgcggacatcaccctggagagcggtcctgacaccgcgcaacatatcctggacctgacctccgctcgtggtgccgaagtgatttttgacttcgtcggtatcgatgcgacggcacagatgagcgtccaagcggtagccccgaatggcgcataccgtatggttggtctgggtggtggcaacccgggcattactgcagaggcagcgggtggtcctggttggccgtggggtgcttcgatccgcaaaagctatggcggcacgcgtaacgacctggttgattctattgcgttggcccaggctggtcttgttaccgttgaagtggcgcgctttgacctggcagacgcccgtgatgcgctggaccgtctggagcatggtaaagtgacgggtcgcgctgtgctggtgccgtaa

SEQ ID NO: 154 - DNA, 1047 bp, Rhodococcus rhodochrous
  source 1..1047: mol_type = other DNA
  CDS 1..1047
atgaaggcaatccagtacacgagaatcggcgcagaacccgaactcacggagattcccaaacccgagcccggtccaggtgaagtgctcctggaagtcaccgctgccggcgtctgccactcggacgacttcatcatgagcctgcccgaagagcagtacacctacggccttcctctcacgctcggccacgaaggcgccggccgggtcgccgccgtcggcgagggcgtcgaaggactcgacatcggaaccaatgtcgtcgtctacggaccctggggctgtggcagctgttggcactgctcgcaaggactcgaaaactactgttctcgggcaaaagaactcggcatcaatcctcctggtctcggtgcacccggcgcgttggccgaattcatgatcgtcgattcacctcgccacctcgtcccgatcggcgacctcgatccggtcaagacggtgccgctgaccgacgccggtctgactccgtatcacgcgatcaagcgttcactgccgaaacttcgcggtggcgcgtacgccgtcgtcatcggtaccggcggtctcggccatgtcgccatccaactcctccgccacctctcggcagcaaccgtcatcgcactcgacgtgagcgcggacaagctcgaactggcaaccaaggtaggcgctcacgaagtggtcctgtccgacaaggacgcggccgagaacgtccgcaggatcaccggaagtcagggcgccgcactggttctcgacttcgtcggctatcagcccaccatcgacaccgcgatggctgtcgccggcgtcggatcggacgtcacgatcgtcgggatcggcgacgggcaggcccatgccaaagtcgggttcttccaaagtccttacgaggcttctgtgacagttccgtactggggtgcccgcaacgagctgatcgaattgatcgacctggcgcacgccggcatcttcgacatcgcggtggagaccttcagtctcgacaacggcgccgaagcgtatcgacgactggccgccggaacgctcagcggccgcgcggttgtggtccctggtctgtga

SEQ ID NO: 155 - protein, 348 aa, Rhodococcus rhodochrous
MKAIQYTRIGAEPELTEIPKPEPGPGEVLLEVTAAGVCHSDDFIMSLPEEQYTYGLPLTLGHEGAGRVAAVGEGVEGLDIGTNVVVYGPWGCGSCWHCSQGLENYCSRAKELGINPPGLGAPGALAEFMIVDSPRHLVPIGDLDPVKTVPLTDAGLTPYHAIKRSLPKLRGGAYAVVIGTGGLGHVAIQLLRHLSAATVIALDVSADKLELATKVGAHEVVLSDKDAAENVRRITGSQGAALVLDFVGYQPTIDTAMAVAGVGSDVTIVGIGDGQAHAKVGFFQSPYEASVTVPYWGARNELIELIDLAHAGIFDIAVETFSLDNGAEAYRRLAAGTLSGRAVVVPGL

SEQ ID NO: 156 - DNA, 1047 bp, Rhodococcus rhodochrous
  source 1..1047: mol_type = other DNA
atgaaagcaatccaatatacccgcattggtgcagagcctgagttgaccgagatcccgaaaccggaaccgggtcctggcgaagttctgctcgaagttaccgctgcgggtgtgtgccacagcgatgactttatcatgtcgctgccagaggaacaatacacgtacggcttaccgctgacgctgggtcatgagggcgctggtcgtgttgcagcggtgggtgagggtgtcgagggcctggacattggcaccaacgttgtcgtgtacggtccgtggggttgcggctcttgttggcattgctcccagggcctggagaattactgttcccgcgcgaaagaactgggtatcaatccgcctggtctgggtgctccaggtgcgctggctgagttcatgattgtcgatagcccgcgtcacttggttccgatcggtgacctggacccggtgaaaaccgtcccgctgaccgatgcgggcttgacgccgtatcacgcgattaagcgcagcctgccgaaactgcgtggtggcgcgtatgcagtcgtcatcggtactggtggcttgggccatgttgcgattcagctgctgcgtcatctgtctgccgcgacggttatcgcgctggacgtgagcgccgataagctcgaactggccactaaggttggcgcgcacgaagtcgttctgagcgataaagacgcagccgaaaatgtgcgtcgtattaccggtagccagggtgcagcattggttctggacttcgttggttatcagccgacgatcgacaccgcgatggccgttgcgggcgttggtagcgatgtcaccattgtgggtattggcgatggtcaagcccacgccaaggttggtttctttcaaagcccgtatgaagcgagcgtcacggtgccgtactggggtgcgcgcaacgaactgatcgagctgatcgatctggctcacgcgggtattttcgacatcgcagtcgaaacctttagcctggataacggcgcagaggcataccgtcgtctggcggctggcactctgagcggtcgcgcagtggtagtgccgggtctgtaa

SEQ ID NO: 157 - DNA, 1062 bp, Rhodococcus rhodochrous
  source 1..1062: mol_type = other DNA
  CDS 1..1062
atgttggcagtccagctgacggcgtggggtcagcctccgcaggtgcgtgagatccccgtacccgagcccgctgaggggcaactgttgatcaaagtcggcgccgctggtctgtgccgctcggatctgcacgtcatggattcgcccgccggacgtttcgattacccgttgccgctcacactcggccatgaggttgccggtaccgtcgtcggtgcgggaccgctggccgatcacgcgtggatcggtgaaaatgttgtcattcatggtgtttggccatgtggccggtgccgcaattgccggcgcgagcgcgagaactactgcttggagaaagtcccgcgtggggacggccgactcagcccgatcggaaacgggttgggccatccgggcgggctggcagaatacctgctggtgccctcggaagctgttctcgttcgcgtcggttcgctgagcccccagcaggccgctccgctcgccgacgccggcctgaccgcatatcatgcgatccggaccaacagcgacgtcatcgactcggacactgtggctttggtgataggaatcggcggtctcggccatctggcggtgcagatcctgcgctctttcggcgtcacagacatcatcgccgtcgagacaagaacccagacacacgctctcgcgctcgaatcgggagcacacgcgtgttttgcgacgctcgcggaagcgacagaggctgtggcgagcctcggcggtgccgacgtggtcttcgactttgtcggggctcaggcgacggtcgaacccgctccggcgcttctcgctcccggcggccgagttgtcgtcgtgggaagtgcgggcgggcaactgaccgtcggcaaaagccttggtttggtcaacggctggcaagttcgggcgccgttctggggcaccatcgaggacctgcgtcaggtggtcgaactcgccagtgcaggaaagctgcatgccgaggtgaccacgttcacgttcgacagcgcactggaggcatacgatcgcctgcgttcaggcgatctgtccggccgcgccgtactggttcccacagccccttcatcgctgtga

SEQ ID NO: 158 - protein, 353 aa, Rhodococcus rhodochrous
MLAVQLTAWGQPPQVREIPVPEPAEGQLLIKVGAAGLCRSDLHVMDSPAGRFDYPLPLTLGHEVAGTVVGAGPLADHAWIGENVVIHGVWPCGRCRNCRRERENYCLEKVPRGDGRLSPIGNGLGHPGGLAEYLLVPSEAVLVRVGSLSPQQAAPLADAGLTAYHAIRTNSDVIDSDTVALVIGIGGLGHLAVQILRSFGVTDIIAVETRTQTHALALESGAHACFATLAEATEAVASLGGADVVFDFVGAQATVEPAPALLAPGGRVVVVGSAGGQLTVGKSLGLVNGWQVRAPFWGTIEDLRQVVELASAGKLHAEVTTFTFDSALEAYDRLRSGDLSGRAVLVPTAPSSL

SEQ ID NO: 159 - DNA, 1062 bp, Rhodococcus rhodochrous
  source 1..1062: mol_type = other DNA
atgttagctgttcaactcaccgcatggggccaaccaccacaagttcgcgaaatcccggttccggaaccagccgagggccaactgctgattaaggttggcgcagccggtctgtgccgtagcgaccttcacgttatggacagccctgctggtcgttttgattacccgttgccgctgacgctgggtcacgaagtggccggcacggttgtcggtgccggtccgttggcagaccacgcgtggattggtgagaacgtcgtgattcacggtgtgtggccgtgtggccgttgtcgtaattgccgtcgcgagcgtgagaactactgtttggaaaaagtgccgcgtggtgacggtcgtctgtccccgatcggcaatggtctgggtcatccgggtggtctggcagagtatctgctggtgccgagcgaagccgtcctggtgcgtgtcggctctctgagcccgcaacaggcagcaccgctggcagatgcgggtctgaccgcgtatcacgcgattcgcacgaatagcgacgttatcgactctgataccgtggcgctggtcatcggtattggtggcctgggtcacctggccgttcagattctgcgttccttcggcgtgacggacatcattgcagtcgagactcgtacccagacgcatgcgttggccctggagagcggtgcgcatgcgtgctttgcgaccctggcggaagcaaccgaagcggttgcgagcttgggcggtgcagatgttgtctttgacttcgttggtgcgcaggcgactgttgagccggcaccagctctgctggcacctggtggccgtgttgtcgtggtgggttctgcgggtggccaactgaccgtcggcaaatccctgggtctggtgaatggctggcaagtgcgtgcgccgttttggggcaccattgaagatttgcgtcaagtcgtggaactggcgtctgcaggcaagttgcacgccgaagttaccacgttcacgttcgatagcgcgctggaagcgtacgaccgcctgcgtagcggtgatctgagcggtcgcgctgtactggttccgaccgcccctagcagcctgtaa

SEQ ID NO: 160 - DNA, 1122 bp, Rhodococcus erythropolis
  source 1..1122: mol_type = other DNA
  CDS 1..1122
atgattcgcgccgaacagaattcgagatcctccatgcagatgacagcggcgctctcacacggcccgcactcccccttcacgctcgacaccgtcgagatcgacgacccccgcgcagacgagatcctggttcgcatcgtcgcgaccggcctgtgccacacagatctgttcacgaagtcggcgctaccggaaagactcggcccctgcgtgttcgggcacgaaggggcgggggtggtcgaggccgtcggctcgtcgatcgacagcattgcgcccggtgatcacgtgttgctgagctaccgcagttgcggtgtgtgcaggcagtgtctcagcggccatcgggcgtactgcgaaagctcacacgggctcaacagctctggcgcacgcaccgacggctcgacgccgatccggcgagacggaaccccgctacggtccgccttcttcggccagtccagcttcgcggaatacgtcatcgcctctgccgacaacaccgtcgtcgtcgatcctgcggtggacctgaccgtcgcagctccgctcggctgcgggtttcaaaccggcgccggcgcggtactgaatctgcttcgccccgagcccgactcgacgtttgtcgttttcggggcaggcagcgtcggactcgcagcgctgctggcggcgagggctgccggcgtttccaccctggtcgccgtggaccccgttgcgcagcggcgcgcactcgccgaggaattcggcgccgtcactgtcgatccctcgaatgaagatgtgatcgacgcggtccacgccgccaccgacggaggttcgacgcattccctcgacaccaccggaatcggctccgtgatcaatcaagccgtcacatcacttcgagcacggggaacactggcggtagtcggactcggagcatccacggtcgaggtgaacatggccgacatcatgctgagcggaaagacaattcgaggatgcatcgaaggagagtcggaagtctcgacgttcatccccgaactcgtcgaactcttcactggtggccggtttccgatcgaccgcttggtgacgcgctacgcattcgccgacatcaacaaagccgtcgaagatcaagcgtcggggcgcgtcatcaaacccgttctcgtgtggtga

SEQ ID NO: 161 - protein, 373 aa, Rhodococcus erythropolis
MIRAEQNSRSSMQMTAALSHGPHSPFTLDTVEIDDPRADEILVRIVATGLCHTDLFTKSALPERLGPCVFGHEGAGVVEAVGSSIDSIAPGDHVLLSYRSCGVCRQCLSGHRAYCESSHGLNSSGARTDGSTPIRRDGTPLRSAFFGQSSFAEYVIASADNTVVVDPAVDLTVAAPLGCGFQTGAGAVLNLLRPEPDSTFVVFGAGSVGLAALLAARAAGVSTLVAVDPVAQRRALAEEFGAVTVDPSNEDVIDAVHAATDGGSTHSLDTTGIGSVINQAVTSLRARGTLAVVGLGASTVEVNMADIMLSGKTIRGCIEGESEVSTFIPELVELFTGGRFPIDRLVTRYAFADINKAVEDQASGRVIKPVLVW

SEQ ID NO: 162 - DNA, 1122 bp, Rhodococcus erythropolis
  source 1..1122: mol_type = other DNA
atgattagagcagaacagaacagccgcagctccatgcaaatgaccgcggcactgtcacatggtccgcacagcccgtttacgctggatacggttgagattgacgatccacgcgccgacgaaattctggtacgcatcgttgcgactggtctgtgtcatacggacttgtttaccaagagcgcgctgccggagcgcctgggtccgtgcgtgttcggccacgagggtgcgggcgtggttgaggcagttggctctagcattgacagcatcgctccgggtgatcacgtcctgttgtcctaccgtagctgcggcgtctgccgtcagtgcctgagcggccaccgtgcttactgtgagagctcccacggcctgaatagctccggtgctcgtaccgacggtagcaccccgatccgtcgtgatggtacgccgcttcgtagcgcgttcttcggtcaatccagcttcgcggaatatgttatcgcaagcgcagacaacaccgttgtggtcgatccggccgtggacttgaccgttgcagcaccgctgggttgtggctttcagaccggcgccggcgcggtgctgaatctgctgcgccctgagccggacagcactttcgtcgtctttggtgccggcagcgtcggtttggcggcactgctggcggcgcgtgcggcgggtgtttcgaccctggtcgcagttgatccggtcgcgcagcgccgtgcgttggccgaagaatttggtgccgttaccgtcgatccgagcaacgaagatgttattgacgctgtgcacgcggcgaccgacggtggcagcacgcattctctggataccacgggcatcggttctgtgattaaccaagccgtgacctctctgcgtgcgcgtggtactctggctgtggttggcctgggtgctagcacggtcgaggtgaatatggcagacattatgctgagcggtaaaacgatccgtggttgcatcgagggcgagagcgaagtttcgacgtttatcccggaactggtcgagctgttcaccggtggccgtttcccgattgaccgcctggttacccgttatgcattcgccgatatcaacaaagctgtggaagatcaagcgtccggtcgcgtcatcaagccagtgctggtgtggtaa

SEQ ID NO: 163 - DNA, 1122 bp, Rhodococcus rhodochrous
  source 1..1122: mol_type = other DNA
  CDS 1..1122
atgattcgcgccgaacagaattcgacgtccgccatgcagatgacagcggcgctctcacacggcccgcactcccccttcacactcgacaccgtcgagatcgacgaaccccgcgcagacgagatcctggttcgcatcgtcgcgaccggcctgtgccacacagatctgttcacgaagtcggtgctaccggaacgactcggcccctgcgtgttcgggcacgaaggggcgggggtggtcgaggccgtcggctcggcgatcgacaaggtcgtgcccggcgatcacgtgttgttgagctaccgcagttgcggtgtgtgcaggcagtgtctcagcggccatcgggcgtactgcgaaagctcacacgggctcaacagctctggcgcacgcaccgacggctcgacgccggtccggcgaagcggaactccgatacggtccgccttcttcggccagtccagcttcgcggaatacgtcatcgccactgccgacaacaccgtcgtcgtcgatcctgcagtggacctgaccgtcgcggctcccctcggctgcggatttcaaaccggcgcgggtgccgtgctgaatctacttcgccccgagcccgactcgacgtttgtcgtcttcggagccggcagcgtcggactcgcagcgctactggcagcgagggctgccggcgtttccaccctggtcgccgtggaccccgttgcgcagcggcgcgcactcgccgaggaattcggcgccgtcactgtcgatccgaccaccgaggacgcggtcgaagcagtacgcgccgccaccgacggaggttcgacacattccctcgacaccaccggaatcggctccgtgatcaatcaagccgtcacatcacttcgagcacggggaacactggcggtagtcggactcggagcgtccacggtcgagatgaacatggccgacatcatgctgagcggaaagacaattcgaggatgcatcgaaggagagtcggaagtctcgacgttcatccccgaactcgtcgaactcttcactggtggccggtttccgatcgaccgcttggtgacgcgctacgccttctccgacatcaacaaagccgtcgaagatcaagcgtcggggcgcgtcatcaaacccgttctcgtgtggtga

SEQ ID NO: 164 - protein, 373 aa, Rhodococcus rhodochrous
MIRAEQNSTSAMQMTAALSHGPHSPFTLDTVEIDEPRADEILVRIVATGLCHTDLFTKSVLPERLGPCVFGHEGAGVVEAVGSAIDKVVPGDHVLLSYRSCGVCRQCLSGHRAYCESSHGLNSSGARTDGSTPVRRSGTPIRSAFFGQSSFAEYVIATADNTVVVDPAVDLTVAAPLGCGFQTGAGAVLNLLRPEPDSTFVVFGAGSVGLAALLAARAAGVSTLVAVDPVAQRRALAEEFGAVTVDPTTEDAVEAVRAATDGGSTHSLDTTGIGSVINQAVTSLRARGTLAVVGLGASTVEMNMADIMLSGKTIRGCIEGESEVSTFIPELVELFTGGRFPIDRLVTRYAFSDINKAVEDQASGRVIKPVLVW

SEQ ID NO: 165 - DNA, 1122 bp, Rhodococcus rhodochrous
  source 1..1122: mol_type = other DNA
atgattagagcagaacagaacagcaccagcgcgatgcaaatgaccgcggcactgtcacatggtccgcacagcccgtttacgctggatacggttgagattgacgagccacgcgccgacgaaattctggtacgcatcgttgcgactggtctgtgtcatacggacttgtttaccaagagcgtcctgccggagcgcctgggtccgtgcgtgttcggccacgagggtgcgggcgtggttgaggcagttggctctgccattgacaaagttgttccgggtgatcacgtcctgttgtcctaccgtagctgcggcgtctgccgtcagtgcctgagcggccaccgtgcttactgtgagagctcccacggcctgaatagctccggtgctcgtaccgacggtagcaccccggtgcgtcgtagcggtacgccgattcgtagcgcgttcttcggtcaatccagcttcgcggaatatgttatcgcaaccgcagacaacaccgttgtggtcgatccggccgtggacttgaccgttgcagcaccgctgggttgtggctttcagaccggcgccggcgcggtgctgaatctgctgcgccctgagccggacagcactttcgtcgtctttggtgccggcagcgtcggtttggcggcactgctggcggcgcgtgcggcgggtgtttcgaccctggtcgcagttgatccggtcgcgcagcgccgtgcgttggccgaagaatttggtgccgttaccgtcgatccgacgaccgaagatgccgttgaagctgtgcgcgcggcgaccgacggtggcagcacgcattctctggataccacgggcatcggttctgtgattaaccaagccgtgacctctctgcgtgcgcgtggtactctggctgtggttggcctgggtgctagcacggtcgagatgaatatggcagacattatgctgagcggtaaaacgatccgtggttgcatcgagggcgagagcgaagtttcgacgtttatcccggaactggtcgagctgttcaccggtggccgtttcccgattgaccgcctggttacccgttatgcattcagcgatatcaacaaagctgtggaagatcaagcgtccggtcgcgtcatcaagccagtgctggtgtggtaa

SEQ ID NO: 166 - DNA, 1128 bp, Azoarcus toluclasticus
  source 1..1128: mol_type = other DNA
  CDS 1..1128
atgggaagcatccaggattcgctgttcattcgggcacgcgctgccgtgctgcgtacggtgggcgggccgctcgagatcgagaacgtgcgcatcagcccccccaagggcgatgaagtgctggtgcgcatggtcggagtcggcgtatgccataccgacgtggtgtgccgcgacggttttcccgtgccgctgccgattgtgctcgggcacgaaggctccggcatcgtcgaggccgtcggcgagcgcgtgacgaaagtgaagccgggccagcgtgtcgtgctgtcgttcaactcctgcgggcactgcgcgagctgctgcgaggatcacccggcgacctgccaccagatgctgccgctcaacttcggcgcggcgcagcgcgtcgatgggggcacggtgatcgatgcgtccggcgaagcggtgcagagcctcttcttcggtcagtcctcgtttggcacctacgcgctcgcgcgcgaagtgaatacggtcccggttccggacgccgtgccgctcgaaatcctcggcccgctcggttgcgggatccagaccggggcgggtgcggcgatcaattcgctcgcgctgaaaccgggccaatcgctcgcgatcttcggcgggggcagcgtcggcctgagcgcgctgctcggcgcgctcgcggtcggtgccggcccggtggtcgtgattgagcccaacgaacggcgtcgcgcgctggcgctcgatctgggtgcaagccacgccttcgatcccttcaacaccgaggatctcgtcgcgagcatcaaggctgcgaccggcggaggcgtcacgcactcgctcgattcgacgggcctcccccccgtcatcgccaacgcgatcaactgcaccctcccgggcggcaccgtcggcctgctgggggtgccgtcacccgaagccgcggtgcctgtgaccctgctggacctgctcgtgaaaagcgtcaccctgcgcccgatcaccgaaggcgacgcgaacccgcaggaattcatcccgcgcatggtccaactctaccgcgacggcaagttccccttcgacaagctgatcaccacctatcgcttcgacgacattaatcaagccttcaaggcgaccgagaccggagaggcgatcaagccggtgctggtgttctga

SEQ ID NO: 167 - protein, 375 aa, Azoarcus toluclasticus
MGSIQDSLFIRARAAVLRTVGGPLEIENVRISPPKGDEVLVRMVGVGVCHTDVVCRDGFPVPLPIVLGHEGSGIVEAVGERVTKVKPGQRVVLSFNSCGHCASCCEDHPATCHQMLPLNFGAAQRVDGGTVIDASGEAVQSLFFGQSSFGTYALAREVNTVPVPDAVPLEILGPLGCGIQTGAGAAINSLALKPGQSLAIFGGGSVGLSALLGALAVGAGPVVVIEPNERRRALALDLGASHAFDPFNTEDLVASIKAATGGGVTHSLDSTGLPPVIANAINCTLPGGTVGLLGVPSPEAAVPVTLLDLLVKSVTLRPITEGDANPQEFIPRMVQLYRDGKFPFDKLITTYRFDDINQAFKATETGEAIKPVLVF

SEQ ID NO: 168 - DNA, 1128 bp, Azoarcus toluclasticus
  source 1..1128: mol_type = other DNA
atgggttctattcaagattctctgttcatccgtgcacgcgccgctgttctgcgtactgtcggtggcccgctggaaattgaaaacgtccgcattagccctccgaagggtgacgaagtgctcgtgcgtatggttggtgttggtgtgtgccataccgacgttgtgtgtcgcgatggcttcccggttccgctgccgattgtgctgggtcacgagggcagcggtattgtcgaggcagtgggcgagcgtgtgaccaaggttaaaccgggtcagcgtgtcgttttatccttcaatagctgtggtcattgcgcgtcctgctgcgaggaccacccggccacctgtcaccagatgctgccactgaactttggtgcggcgcagcgcgtggatggtggcaccgttatcgacgcgagcggcgaggcagtgcagagcctgttttttggtcaaagctctttcggtacgtatgcattggcgcgtgaagtcaataccgtaccggtgccggatgcagttccgttggaaatcctgggcccgttgggttgcggcatccagacgggtgcgggtgcggctatcaacagcctggcgctgaaacctggtcaatcgctggcaatcttcggtggcggcagcgtcggtctgtccgccctgctgggcgcgctggccgtgggcgcgggcccggtcgttgtcattgagccgaacgaacgtcgtcgtgcgttggcgctggacctgggtgcgagccatgcatttgatccgttcaacactgaagatttggttgcgagcatcaaagccgctacgggtggcggcgttacccacagcctggacagcacgggtctgccgccggtcatcgcgaatgcaatcaactgtaccttgccgggcggcacggtcggtctgctgggcgtcccgagcccagaggctgccgttccggtgacgctgctggatctgctggttaaatcagttaccctgcgtccgattaccgagggtgacgccaatccgcaagaatttattccgcgtatggtccagctgtaccgcgacggtaaatttccgtttgataagctgattacgacctaccgcttcgacgacatcaatcaagcgttcaaggcaaccgaaaccggtgaagcgattaagccagtgctggtgttttaa

SEQ ID NO: 169 - DNA, 852 bp, Aspergillus wentii
  source 1..852: mol_type = other DNA
  CDS 1..852
atggcatctgtaccagctcccccatttgtccacgtcgaaggaatgagcaatttccgatcgataggaggatatccccttgagacagcatcgacaaacaatcaccgctccacgaggcaaggattcgcatttcgcagtgccgatccaacctacgtcacccagaaaggcctggaaaccatcctttcgctcgacatcactcgagcctttgacctccgctcactggaagaagcaaaggcacagcgcgcaaaactccaggccgcctcaggatgtctcgactgcagcatcagccagcacatgatccaccagcccacacccctatttccagatggggactggagtccagaggccgcaggggagcggtatctgcagtacgcccaggctgagggagatgggatatcgggctacgtggaggtctacggaaacatgctcgaggaaggttggatggcgattcgcgagattctgcttcatgtccgggaccggcctacagaggcgtttctatgccattgtagtgcagggaaagatcgtacggggattgtcattgcggttttgttgaaggttgcagggtgctcggatgatcttgtgtgcagagagtatgagttgaccgagatcgggttggctcgacggagggagtttatcgtgcagcatctgcttaagaagccggaaatgaatggatcgagggaactggccgaaagagtggcgggggccaggtatgagaatatgaaggaaacgctggagatggtgcaaactagatatagagggatgaggggctattgcaaggagatttgcggcttgaccgacgaagatctatctattatccaggggaacttgactagtccggagagtcctatcttctaa

SEQ ID NO: 170 - protein, 283 aa, Aspergillus wentii
MASVPAPPFVHVEGMSNFRSIGGYPLETASTNNHRSTRQGFAFRSADPTYVTQKGLETILSLDITRAFDLRSLEEAKAQRAKLQAASGCLDCSISQHMIHQPTPLFPDGDWSPEAAGERYLQYAQAEGDGISGYVEVYGNMLEEGWMAIREILLHVRDRPTEAFLCHCSAGKDRTGIVIAVLLKVAGCSDDLVCREYELTEIGLARRREFIVQHLLKKPEMNGSRELAERVAGARYENMKETLEMVQTRYRGMRGYCKEICGLTDEDLSIIQGNLTSPESPIF

SEQ ID NO: 171 - DNA, 852 bp, Aspergillus wentii
  source 1..852: mol_type = other DNA
atggcgtctgtccctgctccaccgtttgttcatgttgaaggtatgtctaattttcgtagcatcggtggctacccgctggagactgcctccacgaataaccatcgctcgacccgtcaaggcttcgcgtttcgtagcgcggacccgacgtatgtgacgcagaaaggcctggaaaccattctgtccctggatattacccgcgcatttgacttgcgtagcttggaagaagcaaaggcacaacgtgcgaagttgcaggccgcgagcggttgtctggattgcagcattagccaacacatgatccaccaaccgaccccgctgttcccggatggtgactggtccccggaagcggcgggtgagcgctacttgcagtacgcacaagctgagggtgatggtatcagcggttatgtcgaagtttatggtaatatgctggaagagggctggatggcgatccgtgagattctgctgcacgtccgtgaccgcccgaccgaagcattcctgtgccactgttccgccggtaaagatcgtacgggtatcgtgattgctgttctgctcaaagtcgcgggttgcagcgacgacctggtgtgtcgtgagtacgaactgaccgagattggcctggcgcgccgtagagagttcatcgttcagcatctgctgaagaaaccggaaatgaacggcagccgtgagctggcggagcgcgtcgcaggcgcccgttacgagaacatgaaagaaaccctggaaatggtgcagacccgttaccgcggcatgcgcggctattgcaaagaaatctgcggtctgaccgacgaagatctgagcattatccagggtaacctgacgagcccggagagcccgattttctaa

SEQ ID NO: 172 - DNA, 2892 bp, Talaromyces verruculosus
  source 1..2892: mol_type = other DNA
  CDS 1..2892
atgagcccaatggatttacaagaatcagcggcagctttggtgcggcagttgggggagagagtcgaagatcgccgtggttttggattcatgagccctgccatctatgataccgcatgggtctctatgattagcaagacaatcgatgaccaaaaaacatggttgtttgcagaatgtttccagtacattctttctcatcagctcgaagacggtggttgggcaatgtatgcatctgaaatcgacgccatcctaaacacttcggcctcattactatcattaaagagacatctttcaaatccctatcaaattacatctatcacacaagaggatctgtccgcccgcattaacagggctcagaatgctttacagaagcttctcaatgagtggaatgtcgacagcacgctccacgtgggattcgagatcctagttccggccctactcaggtatctcgaagatgagggcatcgcttttgctttttctggtagagagcgcctgcttgagattgagaaacagaaattatcaaagttcaaagcacagtatctataccttccaatcaaagtgacagctttgcattctctggaagcgttcataggcgccattgagtttgataaagtcagtcaccacaaagtcagcggtgcgttcatggcatctccatcatccacagcagcttacatgatgcatgcgacacaatgggatgatgaatgcgaggattacctacgccacgtcattgctcatgcatctgggaaaggatccggaggtgttccaagcgcttttccttccaccatctttgaaagcgtttggcctctatcaactctgctaaaggtgggatatgatctcaactcggcaccttttatcgaaaaaatcagatcatacttgcatgatgcatatattgctgaaaagggaattctcggcttcactccttttgttggcgctgatgcagatgataccgctaccaccatattggtgctcaatcttttgaaccaaccagtctcagtcgacgcgatgttgaaggaatttgaagaagaacatcacttcaaaacctactctcaggagcgcaatcctagtttctcggccaattgtaacgttcttcttgccttactatacagtcaagagccatcgctttatagcgcgcagatcgaaaaagctataaggttcctctataagcaattcacagattcagaaatggacgttcgagacaaatggaatctatcaccatactattcttggatgctcatgacacaagccatcacgcggttgacgactcttcagaagacttcgaaactttcaacattgagagatgattctatcagcaaaggcttgattagtctgctgtttaggatagcttctaccgtggttaaagaccaaaagccaggaggttcttggggcactcgagcttcgaaagaagagactgcctacgcagtgttgattctcacatatgctttctacctcgatgaggttacggagtcgttgcggcatgatatcaagatcgccattgagaatggttgctcattcctatctgaaagaaccatgcagtccgattcggagtggctttgggttgagaaagtcacatataaatcagaggttctttcggaagcatatatcttggccgctcttaaacgggcagctgacttacccgacgaaaatgcagaagcagcccccgtcataaatggaatttctacaaatggatttgagcataccgatagaattaacggcaagcttaaagtcaatggtaccaacggtacaaatggcagtcatgagacaaacggtatcaacggtacgcatgaaattgaacagatcaatggcgtcaacggcacgaatggtcactctgatgtgcctcacgatacaaatggctgggtagaagagccgaccgccatcaatgagacaaatggccactacgtgaatggcacgaatcacgagactccccttaccaacggcatttccaatggagattctgtttccgttcatacagaccactcggacagttactatcagcgcagtgattggacagccgacgaagaacaaattcttctcggtccatttgactacctggagagcctgccaggcaagaatatgcgctcacaactgattcaatcattcaacacatggctcaaagtcccaactgagagcttggatgttattattaaggtgatttcaatgttgcatacggcctctctcttgatcgatgatattcaggatcaatcaatactccgccgcgggcaacctgtagcgcacagcatctttggcacagcgcaagcaatgaactcagggaattatgtctactttctagcccttagggaggttcagaaactacaaaacccgaaagccatcagtatttatgttgactctttgattgatcttcaccgtggccaaggcatggagcttttctggcgggattctctcatgtgcccaaccgaagagcagtaccttgacatggtcgcaaacaaaactggcggcctgttttgccttgctatccaattgatgcaagctgaagccactatccaagtcgacttcataccacttgtccgactactcggcatcatcttccagatttgtgatgattacttgaatctgaagtctacggcctatacagacaacaaagggttgtgtgaggatttgacagagggcaaattctcttttcctatcatccatagcattcgatccaaccctggcaaccgacagctaatcaacatcttgaagcaaaagccacgtgaagacgacatcaaacgctatgctctatcctatatggaaagcaccaactcatttgagtatactcggggtgtcgttagaaaactgaagaccgaggcaatcgatactattcaaggcttggagaagcacggcctggaagagaatattggcattcgaaagatactagctcgcatgtcccttgagctatga

SEQ ID NO: 173 - protein, 963 aa, Talaromyces verruculosus
MSPMDLQESAAALVRQLGERVEDRRGFGFMSPAIYDTAWVSMISKTIDDQKTWLFAECFQYILSHQLEDGGWAMYASEIDAILNTSASLLSLKRHLSNPYQITSITQEDLSARINRAQNALQKLLNEWNVDSTLHVGFEILVPALLRYLEDEGIAFAFSGRERLLEIEKQKLSKFKAQYLYLPIKVTALHSLEAFIGAIEFDKVSHHKVSGAFMASPSSTAAYMMHATQWDDECEDYLRHVIAHASGKGSGGVPSAFPSTIFESVWPLSTLLKVGYDLNSAPFIEKIRSYLHDAYIAEKGILGFTPFVGADADDTATTILVLNLLNQPVSVDAMLKEFEEEHHFKTYSQERNPSFSANCNVLLALLYSQEPSLYSAQIEKAIRFLYKQFTDSEMDVRDKWNLSPYYSWMLMTQAITRLTTLQKTSKLSTLRDDSISKGLISLLFRIASTVVKDQKPGGSWGTRASKEETAYAVLILTYAFYLDEVTESLRHDIKIAIENGCSFLSERTMQSDSEWLWVEKVTYKSEVLSEAYILAALKRAADLPDENAEAAPVINGISTNGFEHTDRINGKLKVNGTNGTNGSHETNGINGTHEIEQINGVNGTNGHSDVPHDTNGWVEEPTAINETNGHYVNGTNHETPLTNGISNGDSVSVHTDHSDSYYQRSDWTADEEQILLGPFDYLESLPGKNMRSQLIQSFNTWLKVPTESLDVIIKVISMLHTASLLIDDIQDQSILRRGQPVAHSIFGTAQAMNSGNYVYFLALREVQKLQNPKAISIYVDSLIDLHRGQGMELFWRDSLMCPTEEQYLDMVANKTGGLFCLAIQLMQAEATIQVDFIPLVRLLGIIFQICDDYLNLKSTAYTDNKGLCEDLTEGKFSFPIIHSIRSNPGNRQLINILKQKPREDDIKRYALSYMESTNSFEYTRGVVRKLKTEAIDTIQGLEKHGLEENIGIRKILARMSLEL

SEQ ID NO: 174 - DNA, 2892 bp, Talaromyces verruculosus
  source 1..2892: mol_type = other DNA
atgagccctatggatttgcaagaaagcgccgcagccctggtccgtcaattgggtgaacgcgttgaggatcgccgcggttttggtttcatgagcccggccatttatgacacggcctgggttagcatgattagcaagaccatcgacgaccaaaaaacttggctgtttgcggagtgcttccagtacattctgtctcaccaactggaagatggtggctgggcgatgtacgcatccgaaatcgatgccatcttgaatacttccgcgtcactgctgtccctgaaacgccacctgtccaacccttaccagatcaccagcatcactcaggaagatctgagcgctcgcatcaaccgcgctcaaaacgccctgcagaaattgctgaacgagtggaacgttgactccacgctgcacgtcggtttcgagattctggttccggcgctgctgcgctatctggaagatgaaggcatcgcgtttgcgttctcgggtcgtgagcgtttgttagagattgagaaacaaaaactgtccaagtttaaagcgcagtatttgtacttaccgattaaggtcaccgcactgcatagcctggaagccttcatcggcgctattgagttcgacaaagtcagccatcacaaagtatccggtgctttcatggcgtcgccgtctagcaccgcagcatacatgatgcatgcgacgcaatgggatgacgaatgtgaggattacttgcgtcacgtgatcgcgcatgcgtcaggtaagggttctggcggcgtgccgagcgcctttccgagcaccatcttcgagagcgtttggccgctgtctactctgctgaaagttggctatgatctgaatagcgctccgttcatcgagaaaattcgtagctacttgcacgatgcctatatcgcagagaaaggtattctcggtttcaccccgttcgttggcgctgacgcggacgacaccgctaccacgattctggtgttgaatctgctgaaccaaccggtgagcgtggacgcgatgttgaaagaatttgaagaggaacatcacttcaagacctacagccaagagcgtaatccgagcttttccgcaaactgtaatgttctgctggcgctgctgtacagccaggaaccgagcctgtacagcgcgcaaatcgaaaaagcgatccgttttctgtataagcaattcaccgactctgagatggatgtgcgcgataaatggaacctgtccccgtattatagctggatgctgatgacccaggccatcacccgtctgacgaccctgcaaaagaccagcaagctgagcacgctgcgtgatgacagcattagcaagggcctgatttctctgctgttccgcattgcatccaccgtggttaaagatcaaaaaccgggtggcagctggggcacgcgtgcgagcaaagaagaaacggcatacgccgtgctgattctgacctacgcgttttatctggacgaggtgaccgagtctctgcgccacgatatcaaaattgcaatcgagaatggttgctcgttcctgagcgagcgcaccatgcaaagcgacagcgagtggctgtgggtcgaaaaggttacctacaagagcgaagtgctgagcgaagcatacatcctggcagctctgaaacgtgcggcagacttgccggatgagaacgctgaggcagccccagtgatcaacggtatctctaccaatggctttgagcacaccgaccgcattaatggtaaactcaaggtcaatggtacgaatggcaccaacggttcccacgaaacgaacggtatcaatggcacccatgagattgagcaaattaatggtgtcaacggcacgaatggccatagcgacgtgccacatgacacgaatggttgggtcgaggaaccgacggcgattaatgaaacgaacggtcactacgttaacggcaccaaccatgagactccgctgaccaatggtattagcaatggtgactccgtgagcgttcacaccgaccatagcgacagctactatcagcgtagcgactggaccgcggatgaagaacagatcctgctgggtccattcgattacctggaatccctgcctggtaaaaatatgcgcagccagctgatccagtctttcaatacgtggctgaaggtcccgaccgagagcttggacgtgattattaaggtcattagcatgctgcacactgctagcctgctgatcgacgatattcaggaccaaagcatcctgcgtcgtggtcagcctgtggcgcactcgatcttcggcaccgcgcaagcgatgaactctggtaactatgtttacttcctggcattgcgtgaagttcagaaattgcaaaacccgaaggctatcagcatttatgtggacagcttgatcgatcttcatcgcggccagggcatggaactgttctggcgtgattctctgatgtgcccgactgaagaacagtatctggacatggtggcgaacaagaccggtggcctgttttgtctggcgattcagctgatgcaggcagaagcgaccattcaggttgattttattccgctggtgcgtctgctgggtatcattttccagatttgcgacgactacctgaacttgaaaagcactgcgtataccgacaacaaaggtctgtgtgaagatcttaccgagggtaaattctccttcccgatcattcacagcatccgtagcaatccgggcaatcgtcagctgatcaatattctgaagcaaaaaccgcgcgaagatgacatcaagcgttacgcactgtcctatatggagagcacgaatagcttcgagtacacccgtggcgtcgtccgtaaattgaaaaccgaagcaattgacacgattcaaggtctggagaagcatggcctggaagaaaacattggtattcgtaagattctggcgcgtatgagcctggaactgtaa

SEQ ID NO: 175 - DNA, 936 bp, Talaromyces cellulolyticus
  source 1..936: mol_type = other DNA
  CDS 1..936
atgtctaatgacaccactagcacggcttctgccggaacagcaacttcttcgcggtttctttctgtgggcggagttgtgaatttccgtgaactgggcggttatccatgtgattctgtccctcctgctcctgcctcaaacggctcaccggacaacgcatctgaagcgatcctttgggttggccactcgtccattcggcctaggtttctctttcgatcggcacagccgtctcagattaccccggccggtattgagacattgatccgccagcttggcatccaggcaatttttgactttcgttcacggacggaaattcagcttgtcgccactcgctatcctgattcgctactcgagatacctggtacgactcgctattccgtgcccgtcttcacggagggcgactattccccggcgtcattagtcaagaggtacggagtgtcctccgatactgcaactgattccacttcctccaaatgtgccaagcctacaggattcgtccacgcatatgaggctatcgcacgcagcgcagcagaaaacggcagttttcgtaaaataacggaccacataatacaacatccggaccggcctatcctgtttcactgtacattgggaaaagaccgaaccggtgtatttgcagcattgttattgagtctttgcggggtaccaaacgacacgatagttgaagactatgctatgactaccgagggatttggggtctggcgagaacatctaattcaacgcctgttacaaagaaaggatgcagctacgcgtgaggatgcagaattcattattgccagccacccggagagtatgaaggcttttctagaagatgtggtagcaaccaagttcggggatgctcgaaattactttatccagcactgtggattgacggaagcggaggttgataagctaattcggacactggtcattgcgaattga

SEQ ID NO: 176 - protein, 311 aa, Talaromyces cellulolyticus
MSNDTTSTASAGTATSSRFLSVGGVVNFRELGGYPCDSVPPAPASNGSPDNASEAILWVGHSSIRPRFLFRSAQPSQITPAGIETLIRQLGIQAIFDFRSRTEIQLVATRYPDSLLEIPGTTRYSVPVFTEGDYSPASLVKRYGVSSDTATDSTSSKCAKPTGFVHAYEAIARSAAENGSFRKITDHIIQHPDRPILFHCTLGKDRTGVFAALLLSLCGVPNDTIVEDYAMTTEGFGVWREHLIQRLLQRKDAATREDAEFIIASHPESMKAFLEDVVATKFGDARNYFIQHCGLTEAEVDKLIRTLVIAN

SEQ ID NO: 177 - DNA, 936 bp, Talaromyces cellulolyticus
  source 1..936: mol_type = other DNA
atgagcaacgacacgaccagcaccgcatccgcaggcaccgcaacttcttcgcgctttctgagcgtcggtggcgtggttaacttccgtgagttgggtggctacccgtgcgacagcgttcctcctgcaccagcaagcaatggtagcccggacaatgcgagcgaagcgattctgtgggttggtcacagcagcattcgtccgcgcttcttgtttcgtagcgcacagccgtcccagatcaccccggccggtattgaaacgctgattcgccaactcggtattcaagcgatctttgactttcgttcccgtaccgagatccaactggtggcaacccgctacccagatagcctgctggaaattccgggcacgactcgttactctgttccggtctttaccgagggcgactacagcccggcttctctggttaagcgttatggtgtctctagcgacacggcaacggatagcaccagctcaaagtgcgcgaaaccgaccggctttgtgcatgcttatgaagcgattgctcgttctgccgcggagaacggtagcttccgcaagatcaccgaccacattatccaacatccggatcgcccgatcctgtttcactgcacgctgggcaaagaccgtaccggtgttttcgcagcgctgctgctgagcttgtgtggtgtcccgaatgacaccatcgtggaagattatgcgatgacgaccgaaggcttcggtgtgtggcgtgagcacttgattcagcgtctgctgcagcgcaaagatgcggctacgcgtgaagatgccgagttcattatcgcgagccatccggagagcatgaaagcgttcctggaagatgtcgttgcgaccaaattcggtgacgcccgcaactactttatccagcactgtggtctgaccgaagccgaagtggataagctgatccgtacgctggtgatcgcgaattaa

SEQ ID NO: 178 - DNA, 1122 bp, Castellaniella defragrans
  source 1..1122: mol_type = other DNA
  CDS 1..1122
atgaacgacacccaggatttcatttccgcgcaggccgccgtgctgcgccaggtcggcgggccgctcgcggtcgagcccgtgcgcatcagcatgcccaaaggcgacgaggtcttgatccgcatcgccggcgtgggcgtctgccacaccgacctggtgtgccgcgacggatttcccgtgccgctgccgatcgtgctcggccacgaaggctccggcaccgtcgaggcggtgggcgagcaggtgcgcacgctcaagcccggcgaccgggtcgtgctgtccttcaattcctgcgggcattgcggcaattgccacgacggccatccgtcgaactgcctgcagatgctgcccctgaacttcggcggcgcgcagcgcgtggacggcggccaggtgctggacggcgccggccatcccgtgcagagcatgttcttcggccagtcctcgttcggcacgcatgccgtggcgcgcgaaatcaatgcggtcaaggtcggcgacgacctgccgctggaactgctgggcccgctgggctgcggcatccagaccggcgcgggcgcggcgatcaattcgctggggatcggcccgggccagtccctggccatcttcggcggtggcggcgtcggcctgagcgcgctgctgggcgcgcgcgccgtcggggcggaccgggtcgtggtgatcgagcccaatgccgcgcgccgggccctggccctggaactgggcgccagccatgccctcgacccgcacgccgaaggcgacctggtggccgcgatcaaggcggccaccggcggcggcgcgacccactcgctggacacgacgggcctgcccccggtcatcggcagcgcgatcgcctgcaccctgccgggcggcaccgtgggcatggtcggactgccggcgcccgatgccccggtgccggcgaccctgctcgatctgctgagcaaaagcgtcaccctgcgcccgatcaccgagggcgacgcggacccgcagcgcttcatcccgcgcatgctggatttccatcgcgcgggcaaattcccgttcgaccggctgatcacccgctaccgtttcgaccagatcaacgaggccctgcacgccaccgagaagggcgaggcgatcaagccggtgctggtgttctga

SEQ ID NO: 179 - protein, 373 aa, Castellaniella defragrans
MNDTQDFISAQAAVLRQVGGPLAVEPVRISMPKGDEVLIRIAGVGVCHTDLVCRDGFPVPLPIVLGHEGSGTVEAVGEQVRTLKPGDRVVLSFNSCGHCGNCHDGHPSNCLQMLPLNFGGAQRVDGGQVLDGAGHPVQSMFFGQSSFGTHAVAREINAVKVGDDLPLELLGPLGCGIQTGAGAAINSLGIGPGQSLAIFGGGGVGLSALLGARAVGADRVVVIEPNAARRALALELGASHALDPHAEGDLVAAIKAATGGGATHSLDTTGLPPVIGSAIACTLPGGTVGMVGLPAPDAPVPATLLDLLSKSVTLRPITEGDADPQRFIPRMLDFHRAGKFPFDRLITRYRFDQINEALHATEKGEAIKPVLVF

SEQ ID NO: 180 - DNA, 1122 bp, Castellaniella defragrans
  source 1..1122: mol_type = other DNA
atgaacgatacgcaggattttattagcgcccaagccgcagtgttacgtcaggtcggtggcccgctggccgttgagcctgttcgtatcagcatgccgaagggtgacgaagtcctgattcgtatcgcgggtgttggtgtgtgccacaccgacttggtgtgccgtgatggcttcccggtgccgctgccaattgtgctgggtcacgagggtagcggtactgtcgaagccgtcggtgaacaagtccgtaccctgaaaccgggcgatcgcgtcgtgctgagctttaacagctgcggtcattgcggtaactgtcacgacggtcacccgagcaattgcctgcagatgctgccgctgaacttcggtggcgcgcaacgcgtggacggtggccaagttttggacggtgcgggtcatccggttcagtccatgtttttcggccagtccagctttggcacccacgcagtagcgcgcgagatcaacgcagtcaaggtcggcgatgatctgccactggaactgctgggtccgttgggttgtggcattcaaaccggtgcgggtgcagctatcaattctctgggcattggtccgggtcagtctctggctatcttcggcggcggcggcgtgggtctgagcgcactgctgggcgcccgtgcggtgggtgccgaccgtgttgttgtcattgagccgaatgcagcgcgccgtgcgctggcattggaactgggtgccagccacgcactggacccgcatgccgagggcgaccttgttgcggcgattaaagctgcgacgggtggcggcgctacgcatagcttggatacgaccggcctgccgccagtcattggctccgcgatcgcgtgtactctgccgggtggcaccgttggtatggttggtctgccggcgccggacgcaccggtccctgcgacgctgttggatctgctgagcaaatcggttaccctgcgtccgattaccgagggtgacgctgacccgcaacgcttcatcccgcgtatgctggatttccatcgtgcgggcaagtttccgttcgaccgcctgatcacccgttaccgctttgatcagatcaatgaagcgctgcacgcgaccgagaaaggtgaagcaatcaaaccggttctggtgttttaa

SEQ ID NO: 181 - DNA, 963 bp, Blakeslea trispora
  source 1..963: mol_type = other DNA
  CDS 1..963
atgttgacctctagcaaatcaattgaatccttccccaagaatgttcaaccttatggcaagcattatcaaaatggcttggaacctgttggaaaaagccaagaagatattctcttggagccattccactatctctgttcgaatcctggtaaagatgtccgaaccaagatgattgaagcgttcaatgcttggctgaaagtacccaaggacgatttgatcgtcatcacacgtgtgattgaaatgcttcatagtgctagtttgttaattgatgatgtggaagatgattccgtgttgcgtcgtggtgttcctgcagctcatcatatatatggtactcctcaaactatcaattgtgctaattacgtgtactttcttgcactgaaagaaattgccaagttgaacaagcccaacatgattactatctataccgatgaattgatcaatttgcacagagggcaaggaatggaattgttttggcgtgacaccttaacttgtcctacagagaaagaatttcttgacatggtaaacgacaaaactggtggcctcttgagattagctgtgaaacttatgcaagaagctagtcaatcgggaactgattatacgggactcgtaagtaagattggtatccatttccaagtacgcgacgattatatgaatttgcagtcaaaaaactatgctgacaacaaaggattctgcgaagacttgacagaaggaaaattctctttccctattatacattcaatccgctctgacccaagcaatcgccagcttttgaacattttaaaacagcgcagtagctctatcgaactcaagcaatttgccttgcagctactggaaaacacaaacactttccaatactgtcgtgatttcttacgtgtcttggaaaaggaagctagagaagaaattaagcttttagggggtaacatcatgttggagaaaattatggatgtcttgagtgtcaatgaataa

SEQ ID NO: 182 - protein, 320 aa, Blakeslea trispora
MLTSSKSIESFPKNVQPYGKHYQNGLEPVGKSQEDILLEPFHYLCSNPGKDVRTKMIEAFNAWLKVPKDDLIVITRVIEMLHSASLLIDDVEDDSVLRRGVPAAHHIYGTPQTINCANYVYFLALKEIAKLNKPNMITIYTDELINLHRGQGMELFWRDTLTCPTEKEFLDMVNDKTGGLLRLAVKLMQEASQSGTDYTGLVSKIGIHFQVRDDYMNLQSKNYADNKGFCEDLTEGKFSFPIIHSIRSDPSNRQLLNILKQRSSSIELKQFALQLLENTNTFQYCRDFLRVLEKEAREEIKLLGGNIMLEKIMDVLSVNE

SEQ ID NO: 183 - DNA, 963 bp, Blakeslea trispora
  source 1..963: mol_type = other DNA
atgttgacatcttctaagtccatcgaatctttcccaaagaacgttcaaccatacggtaaacactatcaaaacggtttagaaccagtcggtaagtctcaagaagacatcttgttggaacctttccactacttatgttctaatccaggtaaggatgttagaaccaagatgattgaagctttcaacgcctggttgaaagtcccaaaggacgatttgattgttatcaccagagtcattgaaatgttgcactccgcttctttgttgattgatgacgtcgaggacgattctgtcttgagaagaggtgtcccagccgcccaccatatctacggtacccctcaaaccatcaactgcgctaactacgtttatttcttggccttgaaagaaatcgccaagttgaacaagccaaatatgattactatttataccgatgaattgatcaacttgcacagaggtcaaggtatggaattgttctggcgtgataccttgacctgcccaactgagaaagagtttttggatatggttaacgataagactggtggtttgttgagattggccgtcaagttgatgcaagaggcttctcaatctggtaccgactatactggtttggtttctaagatcggtatccattttcaagttagagatgactacatgaacttgcaatccaaaaactacgccgataataagggtttctgtgaagatttgaccgaaggtaagttctcctttccaattattcactctatcagatctgacccatccaacagacaattattgaatattttgaagcaaagatcttcttctattgaattgaaacaattcgctttacaattgttagaaaacactaacacttttcaatactgtagagatttcttgagagttttggaaaaggaagccagagaagagatcaaattattgggtggtaacatcatgttggaaaagattatggacgtcttgtctgttaatgaataa

SEQ ID NO: 184 - DNA, 2382 bp, Salvia miltiorrhiza
  source 1..2382: mol_type = other DNA
atggcctccttatcctctacaatcctcagccgctctccggcggcccgccgcagaattacgccggcgtcggctaagcttcaccggccggaatgtttcgccaccagtgcatggatgggcagcagcagtaaaaacctttctctcagctaccaacttaatcacaagaaaatatcagttgccacagtagatgcgccgcaggtgcatgaccacgacggcactaccgttcatcaaggccatgatgcggtgaagaatattgaggatcccattgaatacatcaggacgttgttgaggacgacgggggacgggagaataagcgtgtcgccgtacgacacggcgtgggtggcgatgatcaaggacgtggaggggcgggacggcccccagttcccctccagcctcgagtggatcgtgcagaatcaactcgaggatggatcgtggggcgatcagaagcttttctgcgtctacgatcgcctcgtcaataccatcgcgtgcgtggtagccttgagatcgtggaatgttcatgctcacaaggtcaaaagaggagtgacgtacatcaaggaaaatgtggataaacttatggagggaaatgaggagcacatgacttgtgggttcgaagtggtgtttccggcgcttctacaaaaagcgaaaagcttaggcatcgaagatcttccttacgattctccggcggtgcaggaggtttatcatgtcagggaacaaaagttgaaaaggattccactggagattatgcacaaaataccgacatcattattatttagtttggaagggctcgaaaatttggattgggacaaacttttgaaactgcagtcagccgacggttccttcctcacctctccctcctccaccgccttcgcgttcatgcaaaccaaggatgaaaaatgctaccaattcatcaagaacacgatagacactttcaacggaggagcgccacacacttatcccgtcgacgtgtttggaaggctctgggcgatcgaccggctgcagcgcctcggaatttcccgcttttttgagccggagattgctgattgcttaagccacatccacaaattttggacggataagggagttttcagtgggagagaatcggagttttgcgacattgacgatacatccatgggaatgaggcttatgaggatgcatggatatgatgttgatccaaatgtgctgaggaatttcaagcagaaagatggtaaattctcttgctacggcgggcagatgatcgagtcgccttctccgatatacaatctttacagagcttctcagctccgatttcccggcgaggaaatcctcgaagatgcgaagagattcgcctacgatttcttgaaagaaaaactagccaacaatcagattctggataaatgggttatttctaagcacttgcctgatgagatcaagctcgggctagagatgccgtggctcgccaccctaccccgcgtcgaggcgaagtactacatccagtactacgccggctccggcgacgtgtggatcggaaagacgctgtacaggatgccggagatcagcaacgacacgtaccacgacctagccaagacggatttcaagagatgccaagcgaagcatcagttcgagtggctctacatgcaagaatggtacgagagctgcggcatcgaggaattcgggataagcagaaaggaccttctgctttcctatttcttggcgaccgcgagcatcttcgagctcgagaggaccaacgagcgaatcgcgtgggccaaatcgcagatcatcgctaagatgatcacttctttcttcaacaaggaaactacgtcggaggaggacaagcgagctcttttgaacgagctcggaaacattaatggcctcaacgacacaaacggcgcagggagagaaggtggggccggtagcattgcgctagcgaccctcactcagttcctcgagggattcgacagatacaccagacaccagctgaaaaatgcttggagcgtatggctgacgcagctgcaacatggcgaagcagacgacgcggagctcctaaccaacacgttgaacatctgcgccggccacatcgccttcagggaagaaatactggcgcacaacgagtacaaagctctctccaacctaaccagcaaaatctgtcgacagctttctttcattcaaagcgaaaaggagatgggagtagagggcgagatcgcagcgaaatcgagcataaaaaacaaggaactcgaagaagacatgcaaatgttggtgaagttggtgcttgagaaatatgggggcatagatagaaatataaagaaagcgtttttagcagttgcgaagacttattattacagagcgtatcatgccgccgacaccatagacacacacatgtttaaagtgcttttcgagccagtcgcgtga

SEQ ID NO: 185 - protein, 736 aa, Salvia miltiorrhiza
MATVDAPQVHDHDGTTVHQGHDAVKNIEDPIEYIRTLLRTTGDGRISVSPYDTAWVAMIKDVEGRDGPQFPSSLEWIVQNQLEDGSWGDQKLFCVYDRLVNTIACVVALRSWNVHAHKVKRGVTYIKENVDKLMEGNEEHMTCGFEVVFPALLQKAKSLGIEDLPYDSPAVQEVYHVREQKLKRIPLEIMHKIPTSLLFSLEGLENLDWDKLLKLQSADGSFLTSPSSTAFAFMQTKDEKCYQFIKNTIDTFNGGAPHTYPVDVFGRLWAIDRLQRLGISRFFEPEIADCLSHIHKFWTDKGVFSGRESEFCDIDDTSMGMRLMRMHGYDVDPNVLRNFKQKDGKFSCYGGQMIESPSPIYNLYRASQLRFPGEEILEDAKRFAYDFLKEKLANNQILDKWVISKHLPDEIKLGLEMPWLATLPRVEAKYYIQYYAGSGDVWIGKTLYRMPEISNDTYHDLAKTDFKRCQAKHQFEWLYMQEWYESCGIEEFGISRKDLLLSYFLATASIFELERTNERIAWAKSQIIAKMITSFFNKETTSEEDKRALLNELGNINGLNDTNGAGREGGAGSIALATLTQFLEGFDRYTRHQLKNAWSVWLTQLQHGEADDAELLTNTLNICAGHIAFREEILAHNEYKALSNLTSKICRQLSFIQSEKEMGVEGEIAAKSSIKNKELEEDMQMLVKLVLEKYGGIDRNIKKAFLAVAKTYYYRAYHAADTIDTHMFKVLFEPVA

SEQ ID NO: 186 - DNA, 2211 bp, Salvia miltiorrhiza
  source 1..2211: mol_type = other DNA
atggctactgttgacgctccacaagttcacgaccacgacggtactactgttcaccaaggtcacgacgctgttaagaacatcgaagacccaatcgaatacatcagaactttgttgagaactactggtgacggtagaatctctgtttctccatacgacactgcttgggttgctatgatcaaggacgttgaaggtagagacggtccacaattcccatcttctttggaatggatcgttcaaaaccaattggaagacggttcttggggtgaccaaaagttgttctgtgtttacgacagattggttaacactatcgcttgtgttgttgctttgagatcttggaacgttcacgctcacaaggttaagagaggtgttacttacatcaaggaaaacgttgacaagttgatggaaggtaacgaagaacacatgacttgtggtttcgaagttgttttcccagctttgttgcaaaaggctaagtctttgggtatcgaagacttgccatacgactctccagctgttcaagaagtttaccacgttagagaacaaaagttgaagagaatcccattggaaatcatgcacaagatcccaacttctttgttgttctctttggaaggtttggaaaacttggactgggacaagttgttgaagttgcaatctgctgacggttctttcttgacttctccatcttctactgctttcgctttcatgcaaactaaggacgaaaagtgttaccaattcatcaagaacactatcgacactttcaacggtggtgctccacacacttacccagttgacgttttcggtagattgtgggctatcgacagattgcaaagattgggtatctctagattcttcgaaccagaaatcgctgactgtttgtctcacatccacaagttctggactgacaagggtgttttctctggtagagaatctgaattctgtgacatcgacgacacttctatgggtatgagattgatgagaatgcacggttacgacgttgacccaaacgttttgagaaacttcaagcaaaaggacggtaagttctcttgttacggtggtcaaatgatcgaatctccatctccaatctacaacttgtacagagcttctcaattgagattcccaggtgaagaaatcttggaagacgctaagagattcgcttacgacttcttgaaggaaaagttggctaacaaccaaatcttggacaagtgggttatctctaagcacttgccagacgaaatcaagttgggtttggaaatgccatggttggctactttgccaagagttgaagctaagtactacatccaatactacgctggttctggtgacgtttggatcggtaagactttgtacagaatgccagaaatctctaacgacacttaccacgacttggctaagactgacttcaagagatgtcaagctaagcaccaattcgaatggttgtacatgcaagaatggtacgaatcttgtggtatcgaagaattcggtatctctagaaaggacttgttgttgtcttacttcttggctactgcttctatcttcgaattggaaagaactaacgaaagaatcgcttgggctaagtctcaaatcatcgctaagatgatcacttctttcttcaacaaggaaactacttctgaagaagacaagagagctttgttgaacgaattgggtaacatcaacggtttgaacgacactaacggtgctggtagagaaggtggtgctggttctatcgctttggctactttgactcaattcttggaaggtttcgacagatacactagacaccaattgaagaacgcttggtctgtttggttgactcaattgcaacacggtgaagctgacgacgctgaattgttgactaacactttgaacatctgtgctggtcacatcgctttcagagaagaaatcttggctcacaacgaatacaaggctttgtctaacttgacttctaagatctgtagacaattgtctttcatccaatctgaaaaggaaatgggtgttgaaggtgaaatcgctgctaagtcttctatcaagaacaaggaattggaagaagacatgcaaatgttggttaagttggttttggaaaagtacggtggtatcgacagaaacatcaagaaggctttcttggctgttgctaagacttactactacagagcttaccacgctgctgacactatcgacactcacatgttcaaggttttgttcgaaccagttgcttaa

SEQ ID NO: 187 - DNA, 2358 bp, Salvia sclarea
  source 1..2358: mol_type = other DNA
atgacttctgtaaatttgagcagagcaccagcagcgattacccggcgcaggctgcagctacagccggaatttcatgccgagtgttcatggctgaaaagcagcagcaaacacgcgcccttgaccttgagttgccaaatccgtcctaagcaactctcccaaatagctgaattgagagtaacaagcctggatgcgtcgcaagcgagtgaaaaagacatttcccttgttcaaactccgcataaggttgaggttaatgaaaagatcgaggagtcaatcgagtacgtccaaaatctgttgatgacgtcgggcgacgggcgaataagcgtgtcaccctatgacacggcagtgatcgccctgatcaaggacttgaaagggcgcgacgccccgcagtttccgtcatgtctcgagtggatcgcgcaccaccaactggctgatggctcatggggcgacgaattcttctgtatttatgatcggattctaaatacattggcatgtgtcgtagccttgaaatcatggaaccttcactctgatattattgaaaaaggagtgacgtacatcaaggagaatgtgcataaacttaaaggtgcaaatgttgagcacaggacagcggggttcgaacttgtggttcctacttttatgcaaatggccacagatttgggcatccaagatctgccctatgatcatcccctcatcaaggagattgctgacacaaaacaacaaagattgaaagagatacccaaggatttggtttaccaaatgccaacgaatttactgtacagtttagaagggttaggagatttggagtgggaaaggctactgaaactgcagtcgggcaatggctccttcctcacttcgccgtcgtccaccgccgccgtcttgatgcataccaaagatgaaaaatgtttgaaatacatcgaaaacgccctcaagaattgcgacggaggagcaccacatacttatccagtcgatatcttctcaagactttgggcaatcgataggctacaacgcctaggaatttctcgtttcttccagcacgagatcaagtatttcttagatcacatcgaaagcgtttgggaggagaccggagttttcagtggaagatatacgaaatttagcgatattgatgacacgtccatgggcgttaggcttctcaaaatgcacggatacgacgtcgatccaaatgtactaaaacatttcaagcaacaagatggtaaattttcctgctacattggtcaatcggtcgagtctgcatctccaatgtacaatctttatagggctgctcaactaagatttccaggagaagaagttcttgaagaagccactaaatttgcctttaacttcttgcaagaaatgctagtcaaagatcgacttcaagaaagatgggtgatatccgaccacttatttgatgagataaagctggggttgaagatgccatggtacgccactctaccccgagtcgaggctgcatattatctagaccattatgctggttctggtgatgtatggattggcaagagtttctacaggatgccagaaatcagcaatgatacatacaaggagcttgcgatattggatttcaacagatgccaaacacaacatcagttggagtggatccacatgcaggaatggtacgacagatgcagccttagcgaattcgggataagcaaaagagagttgcttcgctcttactttctggccgcagcaaccatattcgaaccggagagaactcaagagaggcttctgtgggccaaaaccagaattctttctaagatgatcacttcatttgtcaacattagtggaacaacactatctttggactacaatttcaatggcctcgatgaaataattagtagtgccaatgaagatcaaggactggctgggactctgctggcaaccttccatcaacttctagacggattcgatatatacactctccatcaactcaaacatgtttggagccaatggttcatgaaagtgcagcaaggagagggaagcggcggggaagacgcggtgctcctagcgaacacgctcaacatctgcgccggcctcaacgaagacgtgttgtccaacaatgaatacacggctctgtccaccctcacaaataaaatctgcaatcgcctcgcccaaattcaagacaataagattctccaagttgtggatgggagcataaaggataaggagctagaacaggatatgcaggcgttggtgaagttagtgcttcaagaaaatggcggcgccgtagacagaaacatcagacacacgtttttgtcggtttccaagactttctactacgatgcctaccacgacgatgagacgaccgatcttcatatcttcaaagtactctttcgaccggttgtatga

SEQ ID NO: 188 - protein, 723 aa, Salvia sclarea
MASQASEKDISLVQTPHKVEVNEKIEESIEYVQNLLMTSGDGRISVSPYDTAVIALIKDLKGRDAPQFPSCLEWIAHHQLADGSWGDEFFCIYDRILNTLACVVALKSWNLHSDIIEKGVTYIKENVHKLKGANVEHRTAGFELVVPTFMQMATDLGIQDLPYDHPLIKEIADTKQQRLKEIPKDLVYQMPTNLLYSLEGLGDLEWERLLKLQSGNGSFLTSPSSTAAVLMHTKDEKCLKYIENALKNCDGGAPHTYPVDIFSRLWAIDRLQRLGISRFFQHEIKYFLDHIESVWEETGVFSGRYTKFSDIDDTSMGVRLLKMHGYDVDPNVLKHFKQQDGKFSCYIGQSVESASPMYNLYRAAQLRFPGEEVLEEATKFAFNFLQEMLVKDRLQERWVISDHLFDEIKLGLKMPWYATLPRVEAAYYLDHYAGSGDVWIGKSFYRMPEISNDTYKELAILDFNRCQTQHQLEWIHMQEWYDRCSLSEFGISKRELLRSYFLAAATIFEPERTQERLLWAKTRILSKMITSFVNISGTTLSLDYNFNGLDEIISSANEDQGLAGTLLATFHQLLDGFDIYTLHQLKHVWSQWFMKVQQGEGSGGEDAVLLANTLNICAGLNEDVLSNNEYTALSTLTNKICNRLAQIQDNKILQVVDGSIKDKELEQDMQALVKLVLQENGGAVDRNIRHTFLSVSKTFYYDAYHDDETTDLHIFKVLFRPVV

SEQ ID NO: 189 - DNA, 2172 bp, Salvia sclarea
  source 1..2172: mol_type = other DNA
atggcatcccaagcgtccgagaaagatattagcctggttcaaaccccgcataaggtcgaggtcaacgaaaagatcgaagagagcatcgagtacgtccaaaatctgctgatgacgagcggtgacggtcgtatctccgtgtctccgtacgataccgcggtcatcgctctgattaaagatctgaagggtcgcgacgcaccgcagttcccgagctgtctggagtggattgcgcaccaccagttagcggatggtagctggggcgacgagttcttttgtatctatgaccgcattttgaataccctggcgtgcgtcgtcgcactgaaatcttggaatctgcacagcgacattattgaaaaaggcgtgacctacattaaggaaaacgtccataagctgaaaggcgcgaatgttgagcatagaaccgccggttttgagctggttgttccgaccttcatgcagatggcgactgacctgggtattcaggatctgccgtacgatcatcctcttatcaaagaaatcgctgatacgaagcaacagcgcctgaaagaaattccgaaagatttggtttatcagatgccgaccaatctgctgtatagcctggaaggcctgggcgatttagagtgggagcgtttgctgaagctgcagtctggtaatggtagcttcctgacgagcccaagcagcacggcggcagttctgatgcataccaaagacgagaagtgtttgaaatacattgagaatgcgctgaagaactgcgacggtggcgctcctcatacgtatccggttgacatctttagccgcttgtgggcgatcgaccgtttgcaacgtctgggcattagccgtttcttccaacacgagatcaaatactttctggaccacatcgagtcagtctgggaagaaaccggcgtgtttagcggtcgttacacgaagtttagcgacatcgatgacacgagcatgggtgtccgcctgctgaaaatgcacggttacgacgtagacccaaacgtgttgaaacactttaagcagcaagacggcaaattcagctgctacatcggccagagcgtcgagagcgcgagcccgatgtataatctgtaccgtgccgcccagctgcgtttcccgggtgaagaagtgcttgaagaagcaactaaattcgcgtttaacttcctgcaagagatgctggtgaaggatcgcttgcaagagcgttgggttattagcgatcacctgtttgacgagattaagctcggtctgaagatgccgtggtatgctaccctgccgcgtgttgaggccgcttattacctggatcactatgcgggtagcggtgatgtgtggattggtaagtctttttaccgcatgccggagattagcaatgacacctacaaagaattggccatcctggactttaaccgttgtcagactcagcatcagctggagtggattcacatgcaagagtggtatgaccgctgctctctgtccgagtttggtattagcaagcgtgagctgctgcgtagctacttcctggctgccgcaaccattttcgaaccggaacgcacccaagagcgtctgctctgggcaaagacccgcatcctgagcaagatgattaccagcttcgtcaacatctccggtacgaccctgagcctggattacaacttcaacggtttggatgagatcatttccagcgcgaatgaagatcagggtctggcgggtacgctgttggccacgttccatcaactgctggatggtttcgacatttacaccctgcaccaactgaaacacgtctggtcgcaatggtttatgaaagttcagcaaggcgagggctccggcggcgaagatgcggtcctgctggcaaatactctgaatatctgcgcgggtctgaatgaagatgtgctgtcgaacaacgagtataccgcgctgagcacgctgacgaacaagatctgcaaccgtctggcccagatccaggacaacaagattctgcaagtggtggacggcagcatcaaagacaaagaactggaacaggatatgcaggcattggttaaactggtgctgcaggaaaacggtggcgcagtggaccgtaacatccgtcacacgtttctgagcgttagcaagaccttctactatgacgcgtatcacgacgatgaaaccaccgatctgcatatctttaaagtcctgttccgtccggttgtttaa

SEQ ID NO: 190 - DNA, 924 bp, Pantoea agglomerans
  source 1..924: mol_type = other DNA
  CDS 1..924
atggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctga

SEQ ID NO: 191 - protein, 307 aa, Pantoea agglomerans
MVSGSKAGVSPHREIEVMRQSIDDHLAGLLPETDSQDIVSLAMREGVMAPGKRIRPLLMLLAARDLRYQGSMPTLLDLACAVELTHTASLMLDDMPCMDNAELRRGQPTTHKKFGESVAILASVGLLSKAFGLIAATGDLPGERRAQAVNELSTAVGVQGLVLGQFRDLNDAALDRTPDAILSTNHLKTGILFSAMLQIVAIASASSPSTRETLHAFALDFGQAFQLLDDLRDDHPETGKDRNKDAGKSTLVNRLGADAARQKLREHIDSADKHLTFACPQGGAIRQFMHLWFGHHLADWSPVMKIA

SEQ ID NO: 192 - DNA, 924 bp, Pantoea agglomerans
  source 1..924: mol_type = other DNA
atggtttctggttcgaaagcaggagtatcacctcatagggaaatcgaagtcatgagacagtccattgatgaccacttagcaggattgttgccagaaacagattcccaggatatcgttagccttgctatgagagaaggtgttatggcacctggtaaacgtatcagacctttgctgatgttacttgctgcaagagacctgagatatcagggttctatgcctacactactggatctagcttgtgctgttgaactgacacatactgcttccttgatgctggatgacatgccttgtatggacaatgcggaacttagaagaggtcaaccaacaacccacaagaaattcggagaatctgttgccattttggcttctgtaggtctgttgtcgaaagcatttggcttgattgctgcaactggtgatcttccaggtgaaaggagagcacaagctgtaaacgagctatctactgcagttggtgttcaaggtctagtcttaggacagttcagagatttgaatgacgcagctttggacagaactcctgatgctatcctgtctacgaaccatctgaagactggcatcttgttctcagctatgttgcaaatcgtagccattgcttctgcttcttcaccatctactagggaaacgttacacgcattcgcattggactttggtcaagcctttcaactgctagacgatttgagggatgatcatccagagacaggtaaagaccgtaacaaagacgctggtaaaagcactctagtcaacagattgggtgctgatgcagctagacagaaactgagagagcacattgactctgctgacaaacacctgacatttgcatgtccacaaggaggtgctataaggcagtttatgcacctatggtttggacaccatcttgctgattggtctccagtgatgaagatcgcctaa

SEQ ID NO: 193 - DNA, 936 bp, Talaromyces verruculosus
  source 1..936: mol_type = other DNA
  CDS 1..936
atgtctaatgacaccactaccacggcttctgccggaacagcaacttcttcgcggtttctttccgtggggggagttgtgaacttccgtgaactgggcggttacccatgtgattctgtccctcctgctcctgcctcaaacggctcaccggacaatgcatctgaagcgaccctttgggttggccactcgtccattcggcctggatttctgtttcgatcggcacagccgtctcagattaccccggccggtattgagacattgatccgccagcttggcatccagacaatttttgactttcgttcaaggacggaaattgagcttgttgccactcgctatcctgattcgctacttgagatacctggcacgactcgctattccgtgcccgtcttctcggaaggcgactattccccagcgtcattagtcaagaggtacggagtgtcctccgatactgcaaccgattccacttcctccaaaagtgctaagcctacaggattcgtccacgcatatgaggctatcgcacgcagtgcagcagaaaacggcagttttcgtaagataacggaccacataatacaacatccggaccggcctattctgtttcactgtacactggggaaagaccgaaccggtgtgtttgcagcattgttattgagtctttgcggggtaccagacgagacgatagttgaagactatgctatgactaccgagggatttggagcctggcgggaacatctaattcaacgcttgctacaaaggaaggatgcagctacgcgcgaggatgcagaatccattattgccagccccccggagactatgaaggcttttctagaagatgtggtagcagccaagttcgggggtgctcgaaattactttatccagcactgtggatttacggaagctgaggttgataagttaagccatacactggccattacgaattga

SEQ ID NO: 194 - protein, 311 aa, Talaromyces verruculosus
MSNDTTTTASAGTATSSRFLSVGGVVNFRELGGYPCDSVPPAPASNGSPDNASEATLWVGHSSIRPGFLFRSAQPSQITPAGIETLIRQLGIQTIFDFRSRTEIELVATRYPDSLLEIPGTTRYSVPVFSEGDYSPASLVKRYGVSSDTATDSTSSKSAKPTGFVHAYEAIARSAAENGSFRKITDHIIQHPDRPILFHCTLGKDRTGVFAALLLSLCGVPDETIVEDYAMTTEGFGAWREHLIQRLLQRKDAATREDAESIIASPPETMKAFLEDVVAAKFGGARNYFIQHCGFTEAEVDKLSHTLAITN

SEQ ID NO: 195 - DNA, 936 bp, Talaromyces verruculosus
  source 1..936: mol_type = other DNA
atgtctaacgacactactactactgcttctgctggtactgctacttcttctagattcttgtctgttggtggtgttgttaacttcagagaattgggtggttacccatgtgactctgttccaccagctccagcttctaacggttctccagacaacgcttctgaagctactttgtgggttggtcactcttctatcagaccaggtttcttgttcagatctgctcaaccatctcaaatcactccagctggtatcgaaactttgatcagacaattgggtatccaaactatcttcgacttcagatctagaactgaaatcgaattggttgctactagatacccagactctttgttggaaatcccaggtactactagatactctgttccagttttctctgaaggtgactactctccagcttctttggttaagagatacggtgtttcttctgacactgctactgactctacttcttctaagtctgctaagccaactggtttcgttcacgcttacgaagctatcgctagatctgctgctgaaaacggttctttcagaaagatcactgaccacatcatccaacacccagacagaccaatcttgttccactgtactttgggtaaggacagaactggtgttttcgctgctttgttgttgtctttgtgtggtgttccagacgaaactatcgttgaagactacgctatgactactgaaggtttcggtgcttggagagaacacttgatccaaagattgttgcaaagaaaggacgctgctactagagaagacgctgaatctatcatcgcttctccaccagaaactatgaaggctttcttggaagacgttgttgctgctaagttcggtggtgctagaaactacttcatccaacactgtggtttcactgaagctgaagttgacaagttgtctcacactttggctatcactaactaa

SEQ ID NO: 196 - DNA, 14 bp, synthetic construct
  misc_feature 1..14: note = RBS sequence
  source 1..14: mol_type = other DNA
aaggaggtaaaaaa

SEQ ID NO: 197 - protein, 7 aa, synthetic construct
  REGION 1..7: note = BVMO sequence motif 1
  VAR_SEQ 4: note = MISC_FEATURE - Xaa can be any naturally occurring amino acid
GAGXSGL

SEQ ID NO: 198 - protein, 32 aa, synthetic construct
  REGION 1..32: note = BVMO sequence motif 2
  VAR_SEQ 4: note = MISC_FEATURE - Xaa can be any naturally occurring amino acid, particularly H or P
  VAR_SEQ 5: note = MISC_FEATURE - Xaa can be any naturally occurring amino acid, particularly A, D, or E
  VAR_SEQ 6: note = MISC_FEATURE - Xaa can be any naturally occurring amino acid, particularly L or V
  VAR_SEQ 7: note = MISC_FEATURE - Xaa can be any naturally occurring amino acid, particularly G or S
  VAR_SEQ 11: note = MISC_FEATURE - Xaa can be any naturally occurring amino acid, particularly F, L, or Y
  VAR_SEQ 24: note = MISC_FEATURE - Xaa can be any naturally occurring amino acid, particularly A or S
  VAR_SEQ 26: note = MISC_FEATURE - Xaa can be any naturally occurring amino acid, particularly A, C, or N
  VAR_SEQ 28: note = MISC_FEATURE - Xaa can be any naturally occurring amino acid, particularly A or T
  VAR_SEQ 29: note = MISC_FEATURE - Xaa can be any naturally occurring amino acid, particularly W or Y
EKNXXXXGTWXENRYPGCACDVPXHXYXXSFE

SEQ ID NO: 199 - protein, 18 aa, synthetic construct
  REGION 1..18: note = BVMO sequence motif 3
  VAR_SEQ 2: note = MISC_FEATURE - Xaa can be any naturally occurring amino acid, particularly I, L, or V
  VAR_SEQ 5: note = MISC_FEATURE - Xaa can be any naturally occurring amino acid, particularly G, S, or T
  VAR_SEQ 10: note = MISC_FEATURE - Xaa can be any naturally occurring amino acid, particularly A or Q
  VAR_SEQ 12: note = MISC_FEATURE - Xaa can be any naturally occurring amino acid, particularly K or R
  VAR_SEQ 13: note = MISC_FEATURE - Xaa can be any naturally occurring amino acid, particularly W or Y
  VAR_SEQ 15: note = MISC_FEATURE - Xaa can be any naturally occurring amino acid, particularly G, P, or S
LXNAXGILNXWXXPXIPG

SEQ ID NO: 200 - protein, 22 aa, synthetic construct
  REGION 1..22: note = BVMO sequence motif 4
  VAR_SEQ 2: note = MISC_FEATURE - Xaa can be any naturally occurring amino acid, particularly E, K, or N
  VAR_SEQ 3: note = MISC_FEATURE - Xaa can be any naturally occurring amino acid, particularly D or G
  VAR_SEQ 5: note = MISC_FEATURE - Xaa can be any naturally occurring amino acid, particularly K, T, or V
  VAR_SEQ 7: note = MISC_FEATURE - Xaa can be any naturally occurring amino acid, particularly A or G
  VAR_SEQ 8: note = MISC_FEATURE - Xaa can be any naturally occurring amino acid, particularly L or V
  VAR_SEQ 11: note = MISC_FEATURE - Xaa can be any naturally occurring amino acid, particularly N or S
  VAR_SEQ 19: note = MISC_FEATURE - Xaa can be any naturally occurring amino acid, particularly L or V
  VAR_SEQ 21: note = MISC_FEATURE - Xaa can be any naturally occurring amino acid, particularly A or N
LXXKXVXXIGXGSSGIQIXPXI

SEQ ID NO: 201 - protein, 15 aa, synthetic construct
  REGION 1..15: note = BVMO sequence motif 5
  VAR_SEQ 5: note = Xaa can be any naturally occurring amino acid, particularly L or P
  VAR_SEQ 9: note = MISC_FEATURE - Xaa can be any naturally occurring amino acid, particularly P or T
  VAR_SEQ 10: note = MISC_FEATURE - Xaa can be any naturally occurring amino acid, particularly G, H, or N
  VAR_SEQ 14: note = MISC_FEATURE - Xaa can be any naturally occurring amino acid, particularly A or S
GCRRXTPGXXYLEXL

SEQ ID NO: 202 - protein, 17 aa, synthetic construct
  REGION 1..17: note = BVMO sequence motif 6
  VAR_SEQ 7: note = MISC_FEATURE - Xaa can be any naturally occurring amino acid, particularly T or V
  VAR_SEQ 8: note = MISC_FEATURE - Xaa can be any naturally occurring amino acid, particularly S or T
  VAR_SEQ 9: note = MISC_FEATURE - Xaa can be any naturally occurring amino acid, particularly F or Y
  VAR_SEQ 10: note = MISC_FEATURE - Xaa can be any naturally occurring amino acid, particularly K or R
  VAR_SEQ 14: note = MISC_FEATURE - Xaa can be any naturally occurring amino acid, particularly K or P
  VAR_SEQ 15: note = MISC_FEATURE - Xaa can be any naturally occurring amino acid, particularly F or L
  VAR_SEQ 16: note = MISC_FEATURE - Xaa can be any naturally occurring amino acid, particularly I or V
CATGFDXXXXPRFXXXG

SEQ ID NO: 203 - protein, 18 aa, synthetic construct
  REGION 1..18: note = BVMO sequence motif 7
  VAR_SEQ 3: note = MISC_FEATURE - Xaa can be any naturally occurring amino acid, particularly S or Y
  VAR_SEQ 5: note = MISC_FEATURE - Xaa can be any naturally occurring amino acid, particularly F, I, or S
  VAR_SEQ 6: note = MISC_FEATURE - Xaa can be any naturally occurring amino acid, particularly F, I, or T
  VAR_SEQ 7: note = MISC_FEATURE - Xaa can be any naturally occurring amino acid, particularly L or M
  VAR_SEQ 11: note = MISC_FEATURE - Xaa can be any naturally occurring amino acid, particularly C or G
  VAR_SEQ 13: note = MISC_FEATURE - Xaa can be any naturally occurring amino acid, particularly I or V
  VAR_SEQ 14: note = MISC_FEATURE - Xaa can be any naturally occurring amino acid, particularly A or G
  VAR_SEQ 17: note = MISC_FEATURE - Xaa can be any naturally occurring amino acid, particularly P or S
PNXFXXXGPNXPXXNGXV

SEQ ID NO: 204 - protein, 21 aa, synthetic construct
  REGION 1..21: note = BVMO sequence motif 8
  VAR_SEQ 2: note = MISC_FEATURE - Xaa can be any naturally occurring amino acid, particularly L or V
  VAR_SEQ 7: note = MISC_FEATURE - Xaa can be any naturally occurring amino acid, particularly A or T
  VAR_SEQ 11: note = MISC_FEATURE - Xaa can be any naturally occurring amino acid, particularly L or M
  VAR_SEQ 14: note = MISC_FEATURE - Xaa can be any naturally occurring amino acid, particularly I or L
  VAR_SEQ 15: note = MISC_FEATURE - Xaa can be any naturally occurring amino acid, particularly A, K, or Q
  VAR_SEQ 16: note = MISC_FEATURE - Xaa can be any naturally occurring amino acid, particularly D, H, or S
  VAR_SEQ 19: note = MISC_FEATURE - Xaa can be any naturally occurring amino acid, particularly W or Y
AXWPGSXLHYXEAXXXPRXED

SEQ ID NO: 205 - protein, 13 aa, synthetic construct
  REGION 1..13: note = enal-cleaving polypeptide sequence motif 1
  VAR_SEQ 2: note = MISC_FEATURE - Xaa can be Y or be deleted
  VAR_SEQ 3: note = MISC_FEATURE - Xaa can be any naturally occurring amino acid
  VAR_SEQ 5: note = MISC_FEATURE - Xaa can be any naturally occurring amino acid
  VAR_SEQ 7: note = MISC_FEATURE - Xaa can be any naturally occurring amino acid
  VAR_SEQ 8: note = MISC_FEATURE - Xaa can be any naturally occurring amino acid
  VAR_SEQ 9: note = MISC_FEATURE - Xaa can be F, L, or I
  VAR_SEQ 10: note = MISC_FEATURE - Xaa can be any naturally occurring amino acid
  VAR_SEQ 11: note = MISC_FEATURE - Xaa can be R, S, or T
  VAR_SEQ 13: note = MISC_FEATURE - Xaa can be H or D
GXXWXGXXXXXGX

SEQ ID NO: 206-207 - skipped (2 entries)

SEQ ID NO: 208 - protein, 12 aa, synthetic construct
  REGION 1..12: note = enal-cleaving polypeptide sequence motif 4
  VAR_SEQ 1: note = MISC_FEATURE - Xaa can be L or M
  VAR_SEQ 2: note = MISC_FEATURE - Xaa can be I or V
  VAR_SEQ 5: note = MISC_FEATURE - Xaa can be any naturally occurring amino acid
  VAR_SEQ 6: note = MISC_FEATURE - Xaa can be any naturally occurring amino acid
  VAR_SEQ 8: note = MISC_FEATURE - Xaa can be I or V
  VAR_SEQ 9: note = MISC_FEATURE - Xaa can be any naturally occurring amino acid
  VAR_SEQ 11: note = MISC_FEATURE - Xaa can be H or S
  VAR_SEQ 12: note = MISC_FEATURE - Xaa can be F or L
XXYDXXPXXDXX